(19) United States
(12) Patent Application Publication
(10) Pub. No.: US 2003/0088059 A1
(43) Pub. Date: May 8, 2003

(54) Human olfactory receptors and genes encoding same

(76) Inventor: Sergey Zozulya, San Diego, CA (US)

Correspondence Address: Pillsbury Winthrop LLP, Intellectual Property Group, East Tower, Ninth Floor, 1100 New York Avenue, N.W., Washington, DC, 20005-3918, US

(21) Appl. No.: 09/804,291

(22) Filed: Mar. 13, 2001

Related U.S. Application Data
(60) Provisional application No. 60/188,914, filed on Mar. 13, 2000.
(60) Provisional application No. 60/192,033, filed on Mar. 24, 2000.
(60) Provisional application No. 60/198,474, filed on Apr. 14, 2000.
(60) Provisional application No. 60/199,335, filed on Apr. 24, 2000.
(60) Provisional application No. 60/207,702, filed on May 26, 2000.
(60) Provisional application No. 60/213,849, filed on Jun. 23, 2000.
(60) Provisional application No. 60/226,534, filed on Aug. 16, 2000.
(60) Provisional application No. 60/230,732, filed on Sep. 7, 2000.
(60) Provisional application No. 60/266,862, filed on Feb. 7, 2001.

(51) Int. Cl. C07K 14/435; C12P 21/02; C12N 5/06; C07H 21/04; C12N 9/00

(52) U.S. Cl. 530/350; 435/183; 435/69.1; 435/325; 435/320.1; 536/23.2

(57) ABSTRACT

Newly identified Olfactory G protein-coupled receptors (ORs), and the genes and cDNA encoding said receptors are described. Specifically, G protein-coupled receptors active in olfactory signaling, and the genes and cDNA encoding the same, are described, along with methods for isolating such genes and for isolating and expressing such receptors. Methods for representing olfactory perception of a particular odorant in a mammal are also described, as are methods for generating novel molecules or combinations of molecules that elicit a predetermined odor perception in a mammal, and methods for simulating one or more odors. Further, methods for stimulating or blocking odor perception in a mammal are also disclosed.

### CROSS REFERENCE TO RELATED APPLICATIONS

This application claims priority to the following provisional applications: U.S. Ser. No. 60/188,914, filed Mar. 13, 2000, entitled, “NOVEL OLFACTORY RECEPTORS AND GENES ENCODING SAME,” to Zozulya; U.S. Ser. No. 60/192,033, filed Mar. 24, 2000, entitled, “NOVEL OLFACTORY RECEPTORS AND GENES ENCODING SAME,” to Zozulya; U.S. Ser. No. 60/198,474, filed Apr. 12, 2000, entitled, “NOVEL OLFACTORY RECEPTORS AND GENE ENCODING THE SAME” to Zozulya; U.S. Ser. No. 60/199,335, filed Apr. 24, 2000, “HUMAN OLFACTORY RECEPTORS AND GENES ENCODING THE SAME”, to Zozulya; U.S. Ser. No. 60/207,702, filed May 26, 2000, entitled, “HUMAN OLFACTORY RECEPTORS AND GENES ENCODING THE SAME”, to Zozulya; U.S. Ser. No. 60/213,849, filed Jun. 23, 2000, entitled, “HUMAN OLFACTORY RECEPTORS AND GENES ENCODING THE SAME”, to Zozulya; U.S. Ser. No. 60/226,534, filed Aug. 16, 2000, “HUMAN OLFACTORY RECEPTORS AND GENES ENCODING THE SAME”, to Zozulya; U.S. Ser. No. 60/230,732, filed Sep. 7, 2000, “HUMAN OLFACTORY RECEPTORS AND GENES ENCODING THE SAME”, to Zozulya; and U.S. Ser. No. 60/266,862, filed Feb. 7, 2001, “HUMAN OLFACTORY RECEPTORS AND GENES ENCODING THE SAME”, to Zozulya. All of these applications are herein incorporated by reference in their entireties.

### FIELD OF THE INVENTION

The invention relates to newly identified mammalian chemosensory G protein-coupled receptors, particularly olfactory receptors, fragments thereof, classes of such receptors, genes and cDNAs encoding said receptors, vectors including said receptors, and cells that express said receptors. The invention also relates to methods of using such receptors, fragments, genes, cDNAs, vectors, and cells to identify molecules involved in olfactory perception. The invention therefore has application in the selection and design of odorant compositions, as well as malodor blockers (olfactory receptor antagonists), particularly perfumes and fragrance compositions and components of deodorants and other malodor blocking compositions.

### DESCRIPTION OF THE RELATED ART

The olfactory system provides sensory information about the chemical composition of the external world. Olfactory sensation is thought to involve distinct signaling pathways. These pathways are believed to be mediated by olfactory receptors (ORs). Cells which express olfactory receptors, when exposed to certain chemical stimuli, elicit olfactory sensation by depolarizing to generate an action potential, which is believed to trigger the sensation.

As such, olfactory receptors specifically recognize molecules that elicit specific olfactory sensation. These molecules are also referred to herein as “odorants.” Olfactory receptors belong to the 7-transmembrane receptor superfamily (Buck et al., Cell 65:175-87 (1991)), which are also known as G protein-coupled receptors (GPCRs). G protein-coupled receptors control many physiological functions, such as endocrine function, exocrine function, heart rate, lipolysis, carbohydrate metabolism, and transmembrane signaling. The biochemical analysis and molecular cloning of a number of such receptors has revealed many basic principles regarding the function of these receptors.

For example, U.S. Pat. No. 5,691,188 describes how upon a ligand binding to a GPCR, the receptor presumably undergoes a conformational change leading to activation of the G protein. G proteins are comprised of three subunits: a guanyl nucleotide binding α subunit, a β subunit, and a γ subunit. G proteins cycle between two forms, depending on whether GDP or GTP is bound to the α subunit. When GDP is bound, the G protein exists as a heterotrimer: the Gαβγ complex. When GTP is bound, the α subunit dissociates from the heterotrimer, leaving a Gαβγ complex. When a Gβγ complex operatively associates with an activated G protein-coupled receptor in a cell membrane, the rate of exchange of GTP for bound GDP is increased and the rate of dissociation of the bound Gα subunit from the Gαβγ complex increases. The free Gα subunit and Gβγ complex are thus capable of transmitting a signal to downstream elements of a variety of signal transduction pathways. These events form the basis for a multiplicity of different cell signaling phenomena, including for example the signaling phenomena that are identified as neurological sensory perceptions such as taste and/or smell.

Genes encoding the olfactory receptors are active primarily in olfactory neurons (Axel, Sci. Amer., 273:154-59 (1995)). Individual olfactory receptor types are expressed in subsets of cells distributed in distinct zones of the olfactory epithelium (Breer, Semin. Cell Biol. 5:25-32 (1994)). The human genome contains approximately one thousand genes that encode a diverse repertoire of olfactory receptors (Rouquier, Nat. Genet., 18:243-50 (1998); Trask, Hum. Mol. Genet., 7:2007-20 (1998)). It has been demonstrated that members of the OR gene family are distributed on all but a few human chromosomes. Through fluorescence in situ hybridization analysis, Rouquier showed that OR sequences reside at more than 25 locations in the human genome. Rouquier also determined that the human genome has accumulated a striking number of dysfunctional OR copies: 72% of the analyzed sequences were found to be pseudogenes. An understanding of an animal's ability to detect and discriminate among the thousands of distinct odorants or tastants, and more particularly to distinguish, for example beneficial tastants or odorants from toxic tastants or odorants, is complicated by the fact that chemosensory receptors belong to a multigene family with over a thousand members. For instance, there are up to 1,000 odorant receptors in mammals.

Moreover, each chemosensory receptor neuron may express only one or a few of these receptors. With respect to odorant receptors, any given olfactory neuron can respond to a small set of odorant ligands. In addition, odorant discrimination for a given neuron may depend on the ligand specificity of the one or few receptors it expresses. To analyze odorant-receptor interactions and their effects on olfactory cells, specific ligands and the olfactory receptors to which they bind are identified. This analysis requires isolation and expression of olfactory polypeptides, followed by binding assays.

Some studies suggest that OR genes can be expressed in tissues other that the olfactory epithelium, indicating potential alternative biological roles for this class of chemosensory receptors. Expression of various ORs has been reported in human and murine erythroid cells (Feingold 1999), developing rat heart (Drutel, Receptor Channels, 3(l):33-40 (1995)), avian notochord (Nef, PNAS, 94(9):4766-71 (1997)) and lingual epithelium (Abe, FES LetI., 316(3):253-56 (1993)). One experimentally documented case also established the existence of a large subset of mammalian ORs transcribed in testes and expressed on the surface of mature spermatozoa, thereby suggesting a possible role of ORs in sperm chemotaxis (Parmenthier, Nature, 355:453-55 (1992); Walensky, Mol. Med., 1(2):130-41 (1998); Branscomb, Genetics, 156(2):785-97 (2000)). It was also hypothesized that olfactory receptors might provide molecular codes for highly specific cell-cell recognition functions in development and embryogenesis (Dreyer, PNAS, 95(11):9072-77 (1998)).

Complete or partial sequences of numerous human and other eukaryotic chemosensory receptors are currently known. See, e.g., Pilpel, Y. and Lancet, D., Protein Science, 8:969-77 (1999); Mombaerts, P., Annu. Rev. Neurosci., 22:487-50 (1999); see also, EP0867508A2, U.S. Pat. No. 5,874,243, WO 92/17585, WO 95/18140, WO 97/17444, WO 99/67282. Due to the complexity of ligand-receptor interactions, and more particularly odorant-receptor interactions, information about ligand-receptor recognition is lacking. In part, the present invention addresses the need for better understanding of these interactions. The present invention also provides, among other things, novel chemosensory receptors, and methods for utilizing such novel chemosensory receptors and the genes and cDNAs encoding such receptors, especially for identifying compounds that can be used to module chemosensory transduction, such as olfaction.

### SUMMARY OF THE INVENTION

Toward that end, it is an object of the invention to provide a new family of G protein-coupled receptors comprising over two hundred fifty olfactory G protein-coupled receptors (OR) active in olfactory perception. It is another object of the invention to provide fragments and variants of such ORs which retain odorant-binding activity.

It is yet another object of the invention to provide nucleic acid sequences or molecules that encode such ORs, fragments, or allelic variants.

It is still another object of the invention to provide expression vectors which include nucleic acid sequences that encode such ORs, or fragments, or variants thereof, which are operably linked to at least one regulatory sequence such as a promoter, enhancer, or other sequences involved in positive or negative gene transcription and/or translation.

It is still another object of the invention to provide human or non-human cells that functionally express at least one of such ORs, or fragments, or variants thereof.

It is still another object of the invention to provide OR fusion proteins or polypeptides which include at least a fragment of at least one of such ORs.

It is another object of the invention to provide an isolated nucleic acid molecule encoding an OR comprising a nucleic acid sequence that is at least 30%, more preferably at least 50%, still more preferably at least 60-70%, and still more preferably 75%, preferably 85%, 90%, 95%, 96%, 97%, 98%, or 99% identical to a nucleic acid sequence selected from the group consisting of: SEQ. ID. NO. 2, SEQ. ID. NO. 4, SEQ. ID. NO. 6, SEQ. ID. NO. 8, SEQ. ID. NO. 10, SEQ. ID. NO. 12, SEQ. ID. NO. 14, SEQ. ID. NO. 16, SEQ. ID. NO. 18, SEQ. ID. NO. 20, SEQ. ID. NO. 22, SEQ. ID. NO. 24, SEQ. ID. NO. 26, SEQ. ID. NO. 28, SEQ. ID. NO. 30, SEQ. ID. NO. 32, SEQ. ID. NO. 34, SEQ. ID. NO. 36, SEQ. ID. NO. 38, SEQ. ID. NO. 40, SEQ. ID. NO. 42, SEQ. ID. NO. 44, SEQ. ID. NO. 46, SEQ. ID. NO. 48, SEQ. ID. NO. 50, SEQ. ID. NO. 52, SEQ. ID. NO. 54, SEQ. ID. NO. 56, SEQ. ID. NO. 58, SEQ. ID. NO. 60, SEQ. ID. NO. 62, SEQ. ID. NO. 64, SEQ. ID. NO. 66, SEQ. ID. NO. 68, SEQ. ID. NO. 70, SEQ. ID. NO. 72, SEQ. ID. NO. 74, SEQ. ID. NO. 76, SEQ. ID. NO. 78, SEQ. ID. NO. 80, SEQ. ID. NO. 82, SEQ. ID. NO. 84, SEQ. ID. NO. 86, SEQ. ID. NO. 88, SEQ. ID. NO. 90, SEQ. ID. NO. 92, SEQ. ID. NO. 94, SEQ. ID. NO. 96, SEQ. ID. NO. 98, SEQ. ID. NO. 100, SEQ. ID. NO. 102, SEQ. ID. NO. 104, SEQ. ID. NO. 106, SEQ. ID. NO. 108, SEQ. ID. NO. 110, SEQ. ID. NO. 112, SEQ. ID. NO. 114, SEQ. ID. NO. 116, SEQ. ID. NO. 118, SEQ. ID. NO. 120, SEQ. ID. NO. 122, SEQ. ID. NO. 124, SEQ. ID. NO. 126, SEQ. ID. NO. 128, SEQ. ID. NO. 130, SEQ. ID. NO. 132, SEQ. ID. NO. 134, SEQ. ID. NO. 136, SEQ. ID. NO. 138, SEQ. ID. NO. 140, SEQ. ID. NO. 142, SEQ. ID. NO. 144, SEQ. ID. NO. 146, SEQ. ID. NO. 148, SEQ. ID. NO. 150, SEQ. ID. NO. 152, SEQ. ID. NO. 154, SEQ. ID. NO. 156, SEQ. ID. NO. 158, SEQ. ID. NO. 160, SEQ. ID. NO. 162, SEQ. ID. NO. 164, SEQ. ID. NO. 166, SEQ. ID. NO. 168, SEQ. ID. NO. 170, SEQ. ID. NO. 172, SEQ. ID. NO. 174, SEQ. ID. NO. 176, SEQ. ID. NO. 178, SEQ. ID. NO. 180, SEQ. ID. NO. 182, SEQ. ID. NO. 184, SEQ. ID. NO. 186, SEQ. ID. NO. 188, SEQ. ID. NO. 190, SEQ. ID. NO. 192, SEQ. ID. NO. 194, SEQ. ID. NO. 196, SEQ. ID. NO. 198, SEQ. ID. NO. 200, SEQ. ID. NO. 202, SEQ. ID. NO. 204. SEQ. ID. NO. 206, SEQ. ID. NO. 208, SEQ. ID. NO. 210, SEQ. ID. NO. 212, SEQ. ID. NO. 214, SEQ. ID. NO. 216, SEQ. ID. NO. 218, SEQ. ID. NO. 220, SEQ. ID. NO. 222, SEQ. ID. NO. 224, SEQ. ID. NO. 226, SEQ. ID. NO. 228, SEQ. ID. NO. 230, SEQ. ID. NO. 232, SEQ. ID. NO. 234, SEQ. ID. NO. 236, SEQ. ID. NO. 238, SEQ. ID NO. 240, SEQ. ID. NO. 242, SEQ. ID. NO. 244, SEQ. ID. NO. 246, SEQ. ID. NO. 248, SEQ. ID. NO. 250, SEQ. ID. NO. 252, SEQ. ID. NO. 254, SEQ. ID. NO. 256, SEQ. ID. NO. 258, SEQ. ID. NO. 260, SEQ. ID. NO. 262, SEQ. ID. NO. 264, SEQ. ID. NO. 266, SEQ. ID. NO. 268, SEQ. ID. NO. 270, SEQ. ID. NO. 272, SEQ. ID. NO. 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370 ,SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512.

It is a further object of the invention to provide an isolated nucleic acid molecule comprising a nucleic acid sequence that encodes a polypeptide having an amino acid sequence which is at least 40%, more preferably at least 50%, still more preferably at least 60-70%, and still more preferably 75%, 85%, 90%, 95%, 96%, 97%, 98%, or 99% identical to an amino acid sequence selected from the group consisting of: SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ID. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 111, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO. 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

It is still a further object of the invention to provide an isolated nucleic acid molecule comprising a nucleic acid sequence that encodes a fragment of a polypeptide having an amino acid sequence selected from the group consisting of: SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ID. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 111, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO. 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511, wherein the fragment is at least 10, preferably 20, 30, 50, 70, 100, or 150 amino acids in length.

It is still a further object of the invention to provide an isolated nucleic acid molecule comprising a nucleic acid sequence that encodes a variant of said fragment, wherein there is a variation in at most 10, preferably 5, 4, 3, 2, or 1 amino acid residues.

It is still another object of the invention to provide an isolated polypeptide comprising an amino acid sequence that is at least 40%, 50%, 60%, 70%, 80%, 90%, 95%, 96%, 97%, 98%, or 99% identical to an amino acid sequence selected from the group consisting of: SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ED. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 111, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. =ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO. 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

It is still a further object of the invention to provide an isolated polypeptide comprising a fragment of a polypeptide having an amino acid sequence selected from the group consisting of: SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ID. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 11, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO. 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511, wherein the fragment is at least 40, preferably 60, 80, 100, 150, 200, or 250 amino acids in length.

It is still a further object of the invention to provide an isolated polypeptide comprising a variant of said fragment, especially naturally occurring allelic variants, the expression of which may be significant in the manner by which different persons in the human population perceive odors differently, both on a qualitative and quantitative level, wherein there is a variation in at most 10, preferably 5, 4, 3, 2, or 1 amino acid residues.

It is still another object of the invention to provide agonists, including inverse agonists, or antagonists of such ORs, or fragments or variants thereof.

It is yet another object of the invention to provide methods for representing the perception of odor and/or for predicting the perception of odor in a mammal, including in a human. Preferably, such methods may be performed by using the ORs, or fragments or variants thereof, and genes encoding such ORs, or fragments or variants thereof, disclosed herein.

It is yet another object of the invention to provide novel molecules or combinations of molecules which elicit a predetermined olfactory perception in a mammal. Such molecules or compositions can be generated by determining a value of olfactory perception in a mammal for a known molecule or combinations of molecules; determining a value of olfactory perception in a mammal for one or more unknown molecules or combinations of molecules; comparing the value of olfactory perception in a mammal for one or more unknown compositions to the value of olfactory perception in a mammal for one or more known compositions; selecting a molecule or combination of molecules that elicits a predetermined olfactory perception in a mammal; and combining two or more unknown molecules or combinations of molecules to form a molecule or combination of molecules that elicits a predetermined olfactory perception in a mammal. The combining step yields a single molecule or a combination of molecules that elicits a predetermined olfactory perception in a mammal.

It is still a further object of the invention to provide a method of screening one or more compounds for the presence of an odor detectable by a mammal, comprising: a step of contacting said one or more compounds with the disclosed ORs, fragments or variants thereof, preferably wherein the mammal is a human.

It is another object of the invention to provided a method for simulating a fragrance, comprising: for each of a plurality of ORs, or fragments of variants thereof disclosed herein, preferably human ORs, ascertaining the extent to which the OR interacts with the fragrance; and combining a plurality of compounds, each having a previously ascertained interaction with one or more of the ORs, in amounts that together provide a receptor-stimulation profile that mimics the profile for the fragrance. Interaction of a fragrance with an OR can be determined using any of the binding or reporter assays described herein. The plurality of compounds may then be combined to form a mixture. If desired, one or more of the plurality of the compounds can be combined covalently. The combined compounds substantially stimulate at least 50%, 60%, 70%, 75%, 80% or 90% or all of the receptors that are substantially stimulated by the fragrance.

In yet another aspect of the invention, a method is provided wherein a plurality of standard compounds are tested against a plurality of ORs, or fragments or variants thereof, to ascertain the extent to which the ORs each interact with each standard compound, thereby generating a receptor stimulation profile for each standard compound. These receptor stimulation profiles may then be stored in a relational database on a data storage medium. The method may further comprise providing a desired receptor-stimulation profile for a scent; comparing the desired receptor stimulation profile to the relational database; and ascertaining one or more combinations of standard compounds that most closely match the desired receptor-stimulation profile. The method may further comprise combining standard compounds in one or more of the ascertained combinations to simulate the scent.

It is a further object of the invention to provide a method for representing olfactory perception of a particular smell in a mammal, comprising: providing values X₁ to Xₙ representative of the quantitative stimulation of each of n ORs of said vertebrate, where n is greater than or equal to 4, n is greater than or equal to 12; n is greater than or equal to 24, n is greater than or equal to 48; n is greater than or equal to 72; n is greater than or equal to 96; n is greater than or equal to 120; n is greater than or equal to 144; n is greater than or equal to 168; n is greater than or equal to 192; n is greater than or equal to 216, or n is greater than or equal to 256; and generating from said values a quantitative representation of olfactory perception. The ORs may be an olfactory receptor disclosed herein, or fragments or variants thereof, the representation may constitutes a point or a volume in n-dimensional space, may constitutes a graph or a spectrum, and may constitutes a matrix of quantitative representations. Also, the providing step may comprise contacting a plurality of recombinantly produced ORs, or fragments or variants thereof, with a test composition and quantitatively measuring the interaction of said composition with said receptors.

It is yet another object of the invention to provide a method for predicting the olfactory perception in a mammal generated by one or more molecules or combinations of molecules yielding unknown olfactory perception in a mammal, comprising: providing values X₁ to Xₙ representative of the quantitative stimulation of each of n ORs of said vertebrate, where n is greater than or equal to 4 n is greater than or equal to 12; n is greater than or equal to 24, n is greater than or equal to 48; n is greater than or equal to 72; n is greater than or equal to 96; n is greater than or equal to 120; n is greater than or equal to 144; n is greater than or equal to 168; n is greater than or equal to 192; n is greater than or equal to 216, or n is greater than or equal to 256; for one or more molecules or combinations of molecules yielding known olfactory perception in a mammal; and generating from said values a quantitative representation of olfactory perception in a mammal for the one or more molecules or combinations of molecules yielding known olfactory perception in a mammal, providing values X₁ to Xₙ representative of the quantitative stimulation of each of n ORs of said vertebrate, where n is greater than or equal to 4, n is greater than or equal to 12; n is greater than or equal to 24, n is greater than or equal to 48; n is greater than or equal to 72; n is greater than or equal to 96; n is greater than or equal to 120; n is greater than or equal to 144; n is greater than or equal to 168; n is greater than or equal to 192; n is greater than or equal to 216, or n is greater than or equal to 273; for one or more molecules or combinations of molecules yielding unknown olfactory perception in a mammal; and generating from said values a quantitative representation of olfactory perception in a mammal for the one or more molecules or combinations of molecules yielding unknown olfactory perception in a mammal, and predicting the olfactory perception in a mammal generated by one or more molecules or combinations of molecules yielding unknown olfactory perception in a mammal by comparing the quantitative representation of olfactory perception in a mammal for the one or more molecules or combinations of molecules yielding unknown olfactory perception in a mammal to the quantitative representation of olfactory perception in a mammal for the one or more molecules or combinations of molecules yielding known olfactory perception in a mammal. The ORs used in this method may include an olfactory receptor, or fragment or variant thereof, disclosed herein.

### BRIEF DESCRIPTION OF THE DRAWINGS

FIG. 1 illustrates the multiple sequence alignment derived for fifty novel ORs, indicating areas of homology and presence of sequence motifs characteristic for olfactory receptors. The fifty novel human olfactory receptors (hOR) proteins described herein are designated AOLFR1 through AOLFR52. The alignment protocol used the Clustal method with PAM250 residue weight table. Amino acid sequences AOLFR2 through AOLFR52 were analyzed for alignment with the AOLFR1 amino acid sequence.

FIG. 2 illustrates the multiple sequence alignment derived for fifty novel ORs, indicating areas of homology and presence of sequence motifs characteristic for olfactory receptors. The fifty novel human olfactory receptors (hOR) proteins described herein are designated AOLFR54 through AOLFR109. The alignment protocol used the Clustal method with PAM250 residue weight table. Amino acid sequences AOLFR55 through AOLFR109 were analyzed for alignment with the AOLFR54 amino acid sequence.

FIG. 3 illustrates the multiple sequence alignment derived for fifty novel ORs, indicating areas of homology and presence of sequence motifs characteristic for olfactory receptors. The fifty novel human olfactory receptors (hOR) proteins described herein are designated AOLFR110 through AOLFR163. The alignment protocol used the Clustal method with PAM250 residue weight table. Amino acid sequences AOLFR111 through AOLFR163 were analyzed for alignment with the AOLF110 amino acid sequence.

FIG. 4 illustrates the multiple sequence alignment derived for fifty-four novel ORs, indicating areas of homology and presence of sequence motifs characteristic for olfactory receptors. The fifty-four novel human olfactory receptors (hOR) proteins described herein are designated AOLFR165 through AOLFR217. The alignment protocol used the Clustal method with PAM250 residue weight table. Amino acid sequences AOLFR166 through AOLFR217 were analyzed for alignment with the AOLFR165 amino acid sequence.

FIG. 5 illustrates the multiple sequence alignment derived for fifty-two novel ORs, indicating areas of homology and presence of sequence motifs characteristic for olfactory receptors. The fifty-two novel human olfactory receptors (hOR) proteins described herein, which are designated AOLFR218 through AOLFR328. The alignment protocol used the Clustal method with PAM250 residue weight table. Amino acid sequences AOLFR219 through AOLFR328 were analyzed for alignment with the AOLFR218 amino acid sequence.

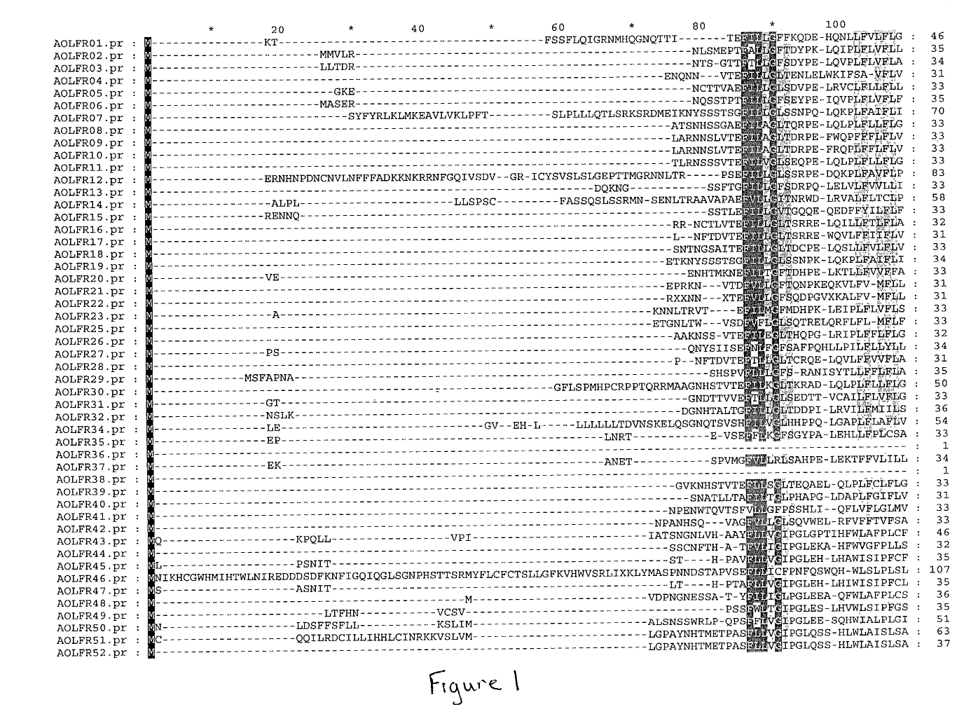

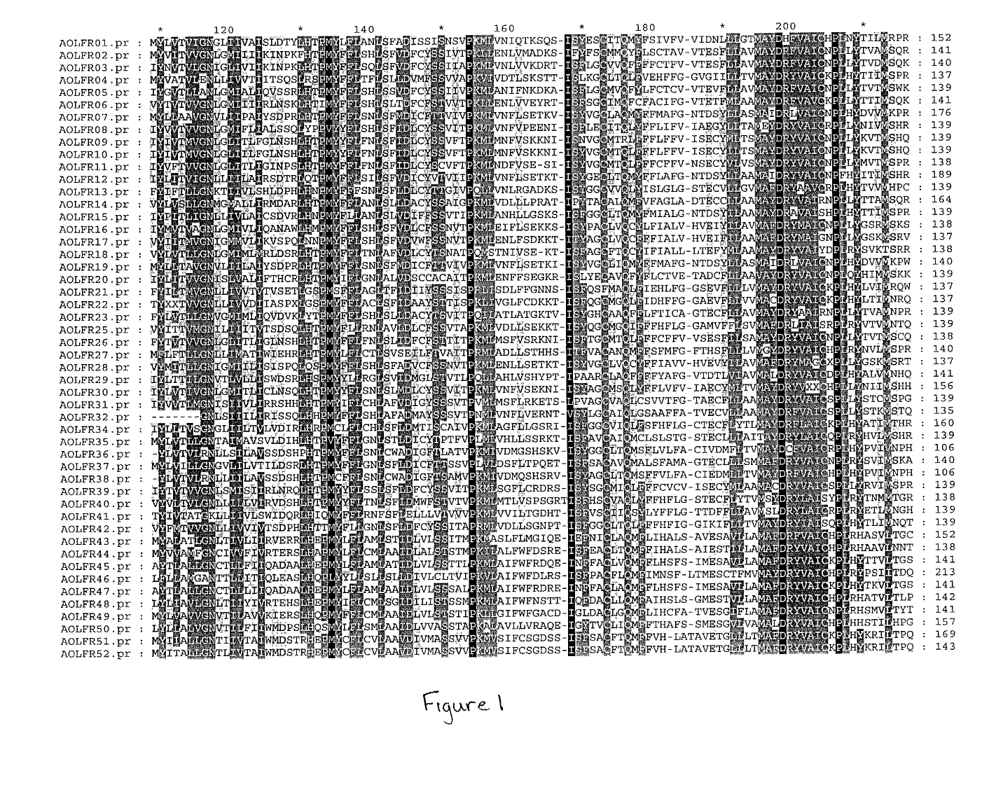

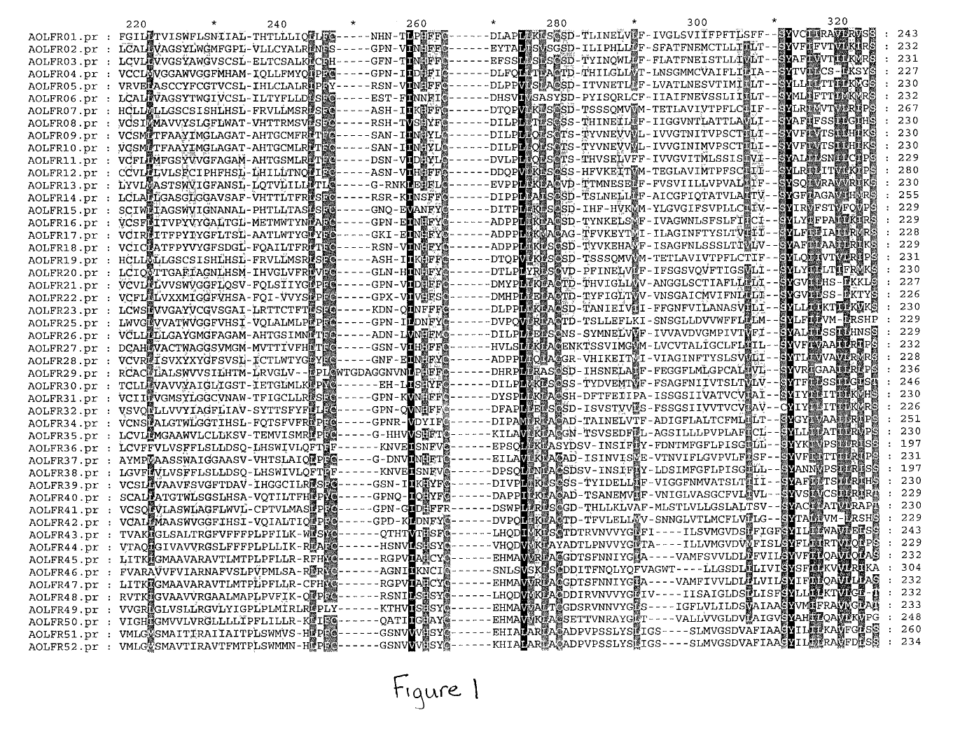

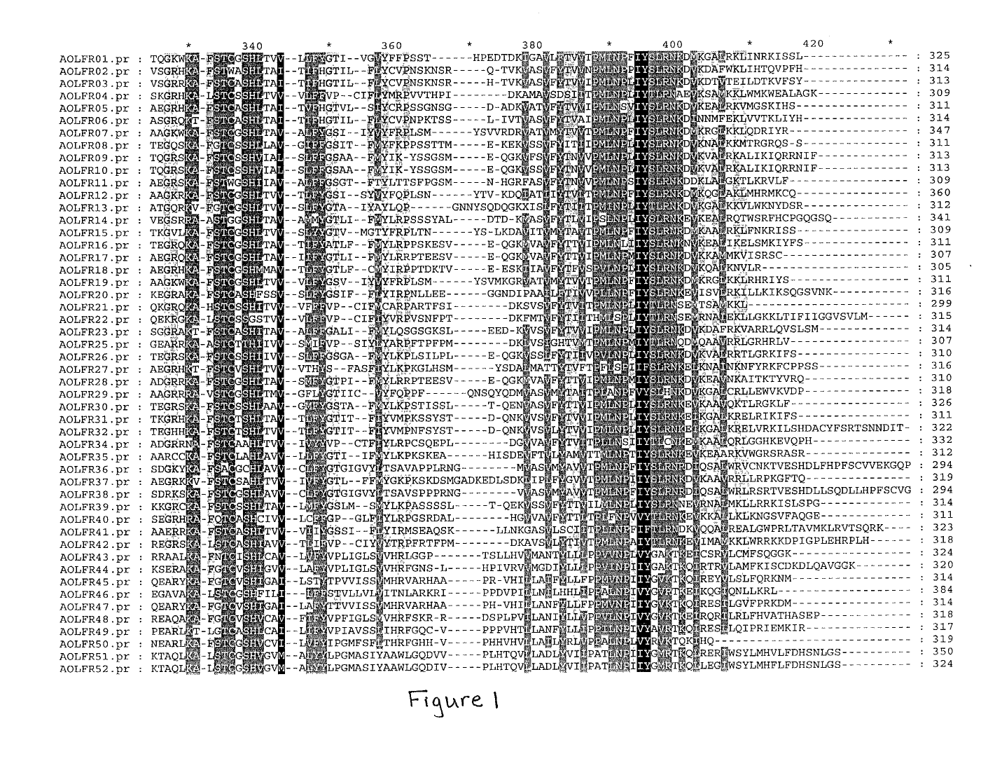

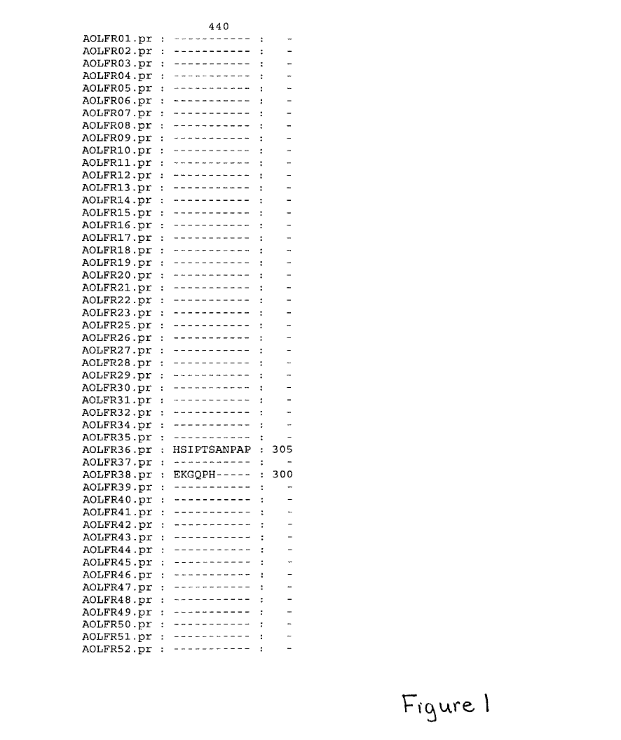

### DETAILED DESCRIPTION OF THE INVENTION

The invention thus provides isolated nucleic acid molecules encoding olfactory-cell-specific G protein-coupled receptors (“GPCRs”), and the polypeptides they encode. These nucleic acid molecules and the polypeptides that they encode are members of the olfactory receptor family. Other members of the olfactory receptor family are disclosed in Krautwurst, et al., Cell, 95:917-26 (1998), and WO 0035274, the contents of which are herein incorporated by reference in their entireties.

According to one aspect of the invention, genes encoding over two hundred fifty distinct, novel human olfactory (odorant) receptors (also herein referred to ORs) have been identified in genome sequence databases. All of these receptor genes have been initially detected by computer DNA sequence analysis of genomic clones (unfinished High Throughput Genomic Sequence database accession numbers AB045359, AP002532, AP002533, AL365440, AC073487, AL359636, AL359955, AP002535, AB045365, AL359218, AC002555, AB045361, AL359512, AC023255, AL358773, AL357767, AL358874, AC068380, AC025283, AP002407, AC018700, AC022289, AC006313, AC002556, AC011571, AL121944, AC007194, AP001112, AC021660, AP000723, AC016856, AC018700, AP000818, AC00596, AP000916, AC011517, AP001112, AP000916, AC021427, AC021427, AC020884, AC019108, AL135841, AL133410, AF186996, AL138834, AC009237, AC025249, AC010930, AC009758, AC009642, AC009758, AC025249, AF101706, AC009642, AC025249, AC021660, AC011647, AC011711, AC09642, AC020597, AC011711, AC019088, AC022882, AC011571, AL121944, AP000435, AC012616, AC010332, AC010766, AP000743, AC021809, AC011879, AC021304, AC023226, AL160314, AC021304, AC020380, AC011904, AC004977, AC021304, AP000868, AP000825, AC023080, AC022207, AC121986, AC010814, AC018700, AC021304, AC008620, AC011537, AC010760, AC027641, AC017103, AC024729, AC024257, AC025115, AP001524, AP000916, AC010814, AL162254, AC025234, AP001521, AC026090, AC019088, AC016856, AC016787, AC009594, AC026038, AQ628489, AC025942, AL163152, AC026975, AC024654, AP001803, AP001804, AL353767, AP001884, AC026083, AC018793, AP000818, AL353894, AL049734, AL355366, AC011464, AC037472, AC036111, AC019093, AC027239, AC027522, AC009545, AC021333, AC036216, AC021935, AC022762, AL356019, AC055861, AC018375, AC072059, AC068339, AC022891, AL357039, AP002345, AC044810, AC073113, AC024399, AC023564, AL390860, AC074365, AP002826, AL359636, AL391534, AC055731, AC076959, AP002826, AC019088, AC009779, AL445307, AP002512, AP000818, AC079190) by virtue of their sequence homology to some of the known human and other mammalian olfactory receptor genes.

Alternatively, nucleic acids encoding the olfactory receptors (ORs) and polypeptides of the invention can be isolated from a variety of sources, genetically engineered, amplified, synthesized, and/or expressed recombinantly according to the methods disclosed in WO 0035374, which is herein incorporated by reference in its entirety.

These nucleic acids provide valuable probes for the identification of olfactory cells, as the nucleic acids are specifically expressed in olfactory cells. They can also serve as tools for the generation of sensory topographical maps that elucidate the relationship between olfactory cells and olfactory sensory neurons leading to olfactory centers in the brain. Furthermore, the nucleic acids and the polypeptides they encode can be used as probes to elucidate olfactory-inducted behaviors.

The invention also provides methods of screening for modulators, e.g., activators, inhibitors, stimulators, enhancers, agonists, inverse agonists and antagonists, of the ORs, or fragments or variants thereof, of the invention. Such modulators of olfactory transduction are useful for pharmacological and genetic modulation of olfactory signaling pathways. These methods of screening can be used to identify high affinity agonists and antagonists of olfactory cell activity. These modulator compounds can then be used in the food, pharmaceutical, and cosmetic industries to customize odors and fragrances.

Thus, the invention provides assays for olfactory modulation, where the ORs, or fragments or variants thereof, of the invention act as direct or indirect reporter molecules for the effect of modulators on olfactory transduction. The ORs, or fragments or variants thereof, can be used in assays, e.g., to measure changes in ion concentration, membrane potential, current flow, ion flux, transcription, signal transduction, receptor-ligand interaction, second messenger concentrations, in vitro, in vivo and ex vivo. In one embodiment, the ORs, or fragments or variants thereof, can be used as an indirect reporters via attachment to second reporter molecules, such as green fluorescent protein (see, e.g., Mistili et al., Nature Biotech., 15:961-64 (1997)). In another embodiment, the ORs, or fragments or variants thereof, can be expressed in host cells, and modulation of olfactory transduction via OR activity can be assayed by measuring changes in Ca²⁺ levels.

Methods of assaying for modulators of olfactory transduction include in vitro ligand binding assays using the ORs of the invention, or fragments or variants thereof. More particularly, such assays can use the ORs; portions thereof such as the extracellular or transmembrane domains; chimeric proteins comprising one or more of such domains; oocyte receptor expression; tissue culture cell receptor expression; transcriptional activation of the receptor; G protein binding to the receptor; ligand binding assays; voltage, membrane potential and conductance changes; ion flux assays; changes in intracellular second messengers such as cAMP and inositol triphosphate; changes in intracellular Ca²⁺ levels; and neurotransmitter release.

The invention also provides for methods of detecting olfactory nucleic acid and protein expression, allowing for the investigation of olfactory transduction regulation and specific identification of olfactory receptor cells. The ORs, fragments, and variants of the invention can also be used to generate monoclonal and polyclonal antibodies useful for identifying olfactory receptor cells. Olfactory receptor cells can be identified using techniques such as reverse transcription and amplification of mRNA, isolation of total RNA or poly A⁺ RNA, northern blotting, dot blotting, in situ hybridization, RNase protection, S1 digestion, probing DNA microchip arrays, western blots, and the like.

A. Identification and Characterization of Olfactory Receptors

The amino acid sequences of the ORs and polypeptides of the invention can be identified by putative translation of the coding nucleic acid sequences. These various amino acid sequences and the coding nucleic acid sequences may be compared to one another or to other sequences according to a number of methods.

For example, in sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, as described below for the BLASTN and BLASTP programs, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of: from 20 to 600, usually about 50 to about 200, more usually about 100 to about 150 in which a sequence may be compared to a reference sequence of the same number of. contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, PNAS, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Altschul et al., Nuc. Acids Res. 25:3389-3402 (1977) and Altschul et al., J Mol. Biol. 215:403-410 (1990)). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, PNAS, 89:10915 (1989)) alignments (B) of 50, expectation (E) of 10, M=5, N=−4, and a comparison of both strands.

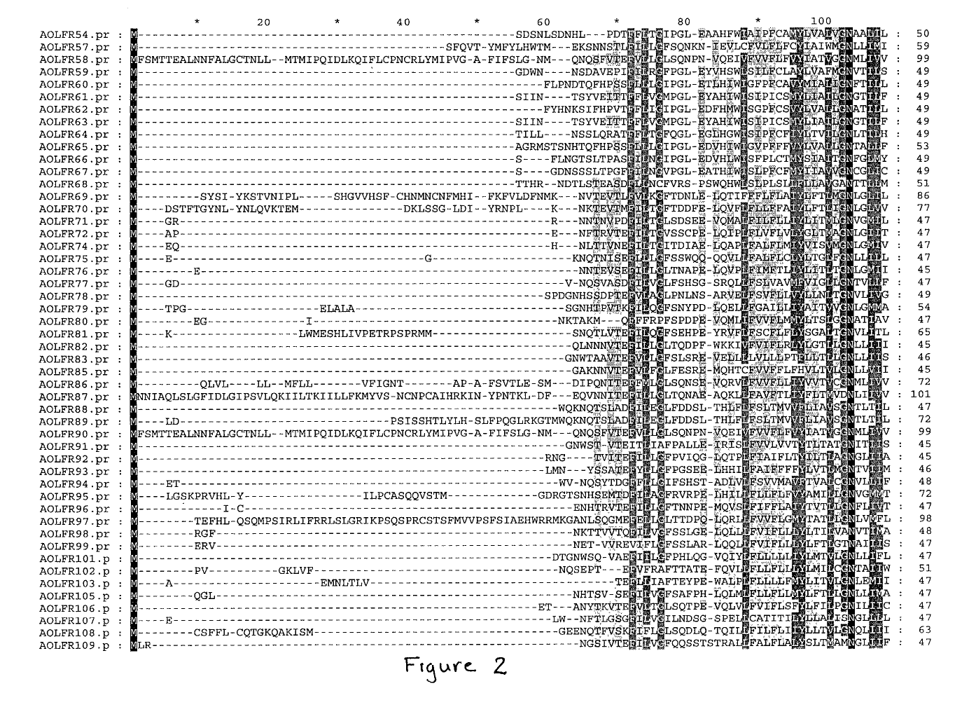

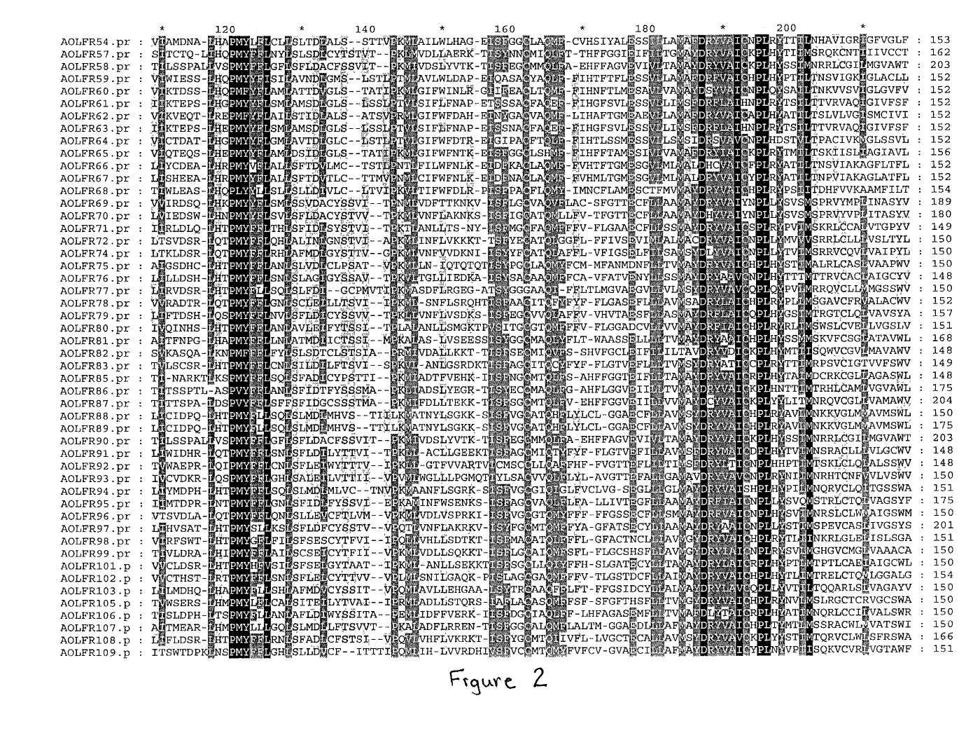

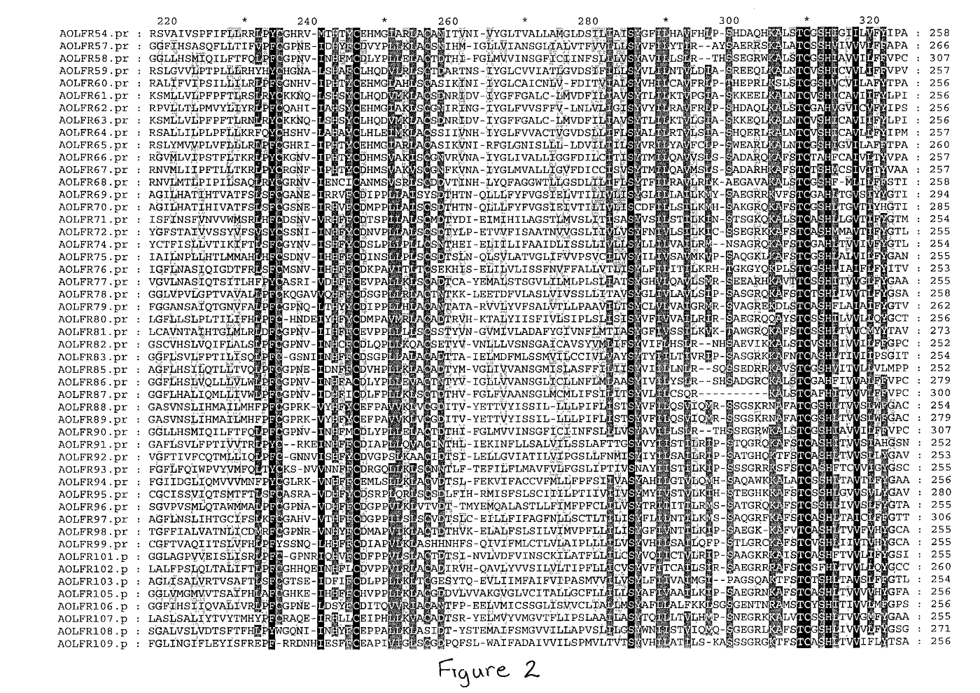

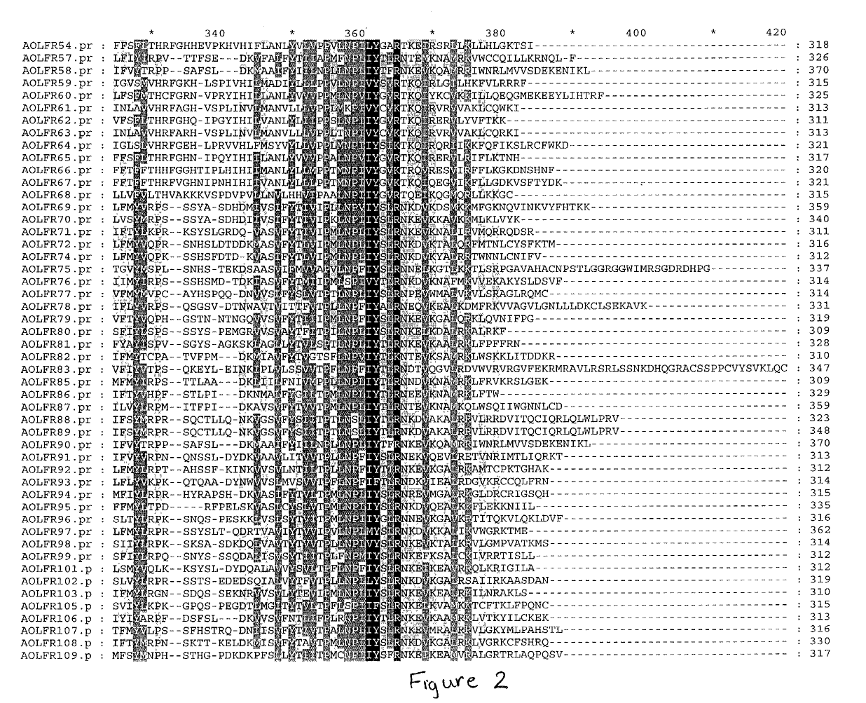

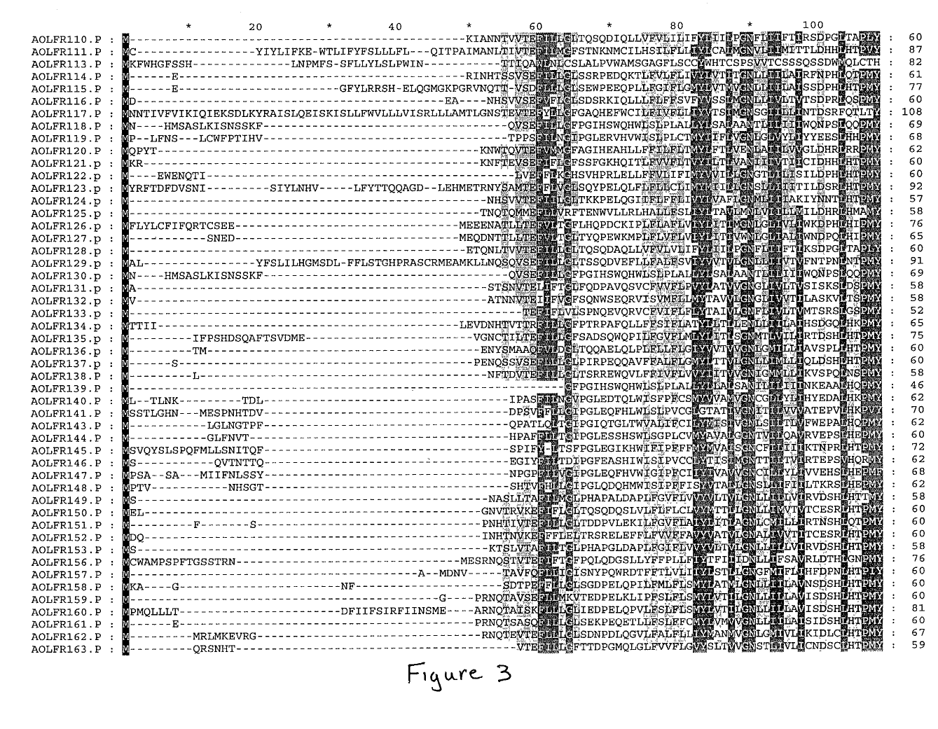

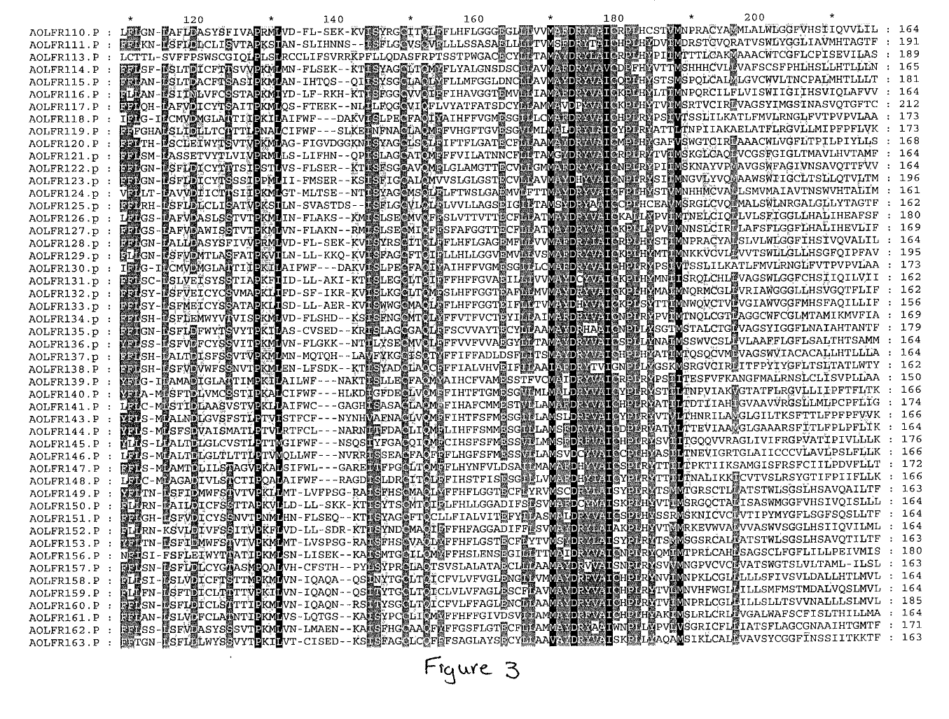

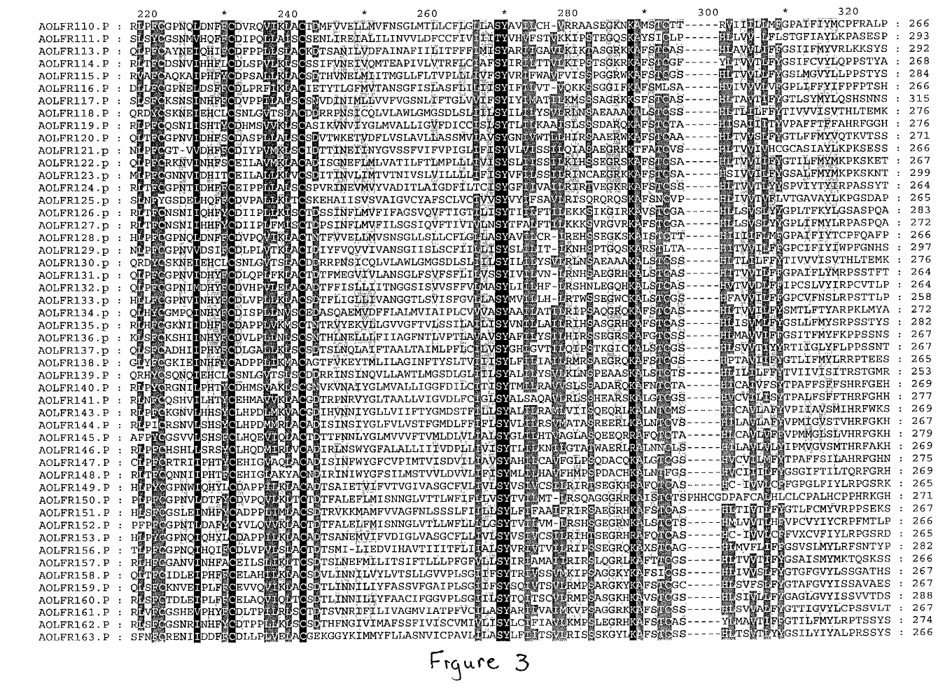

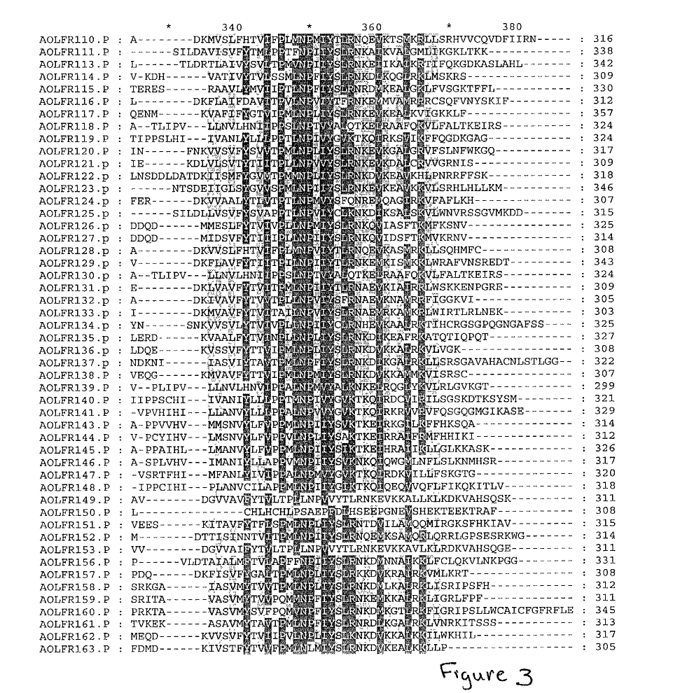

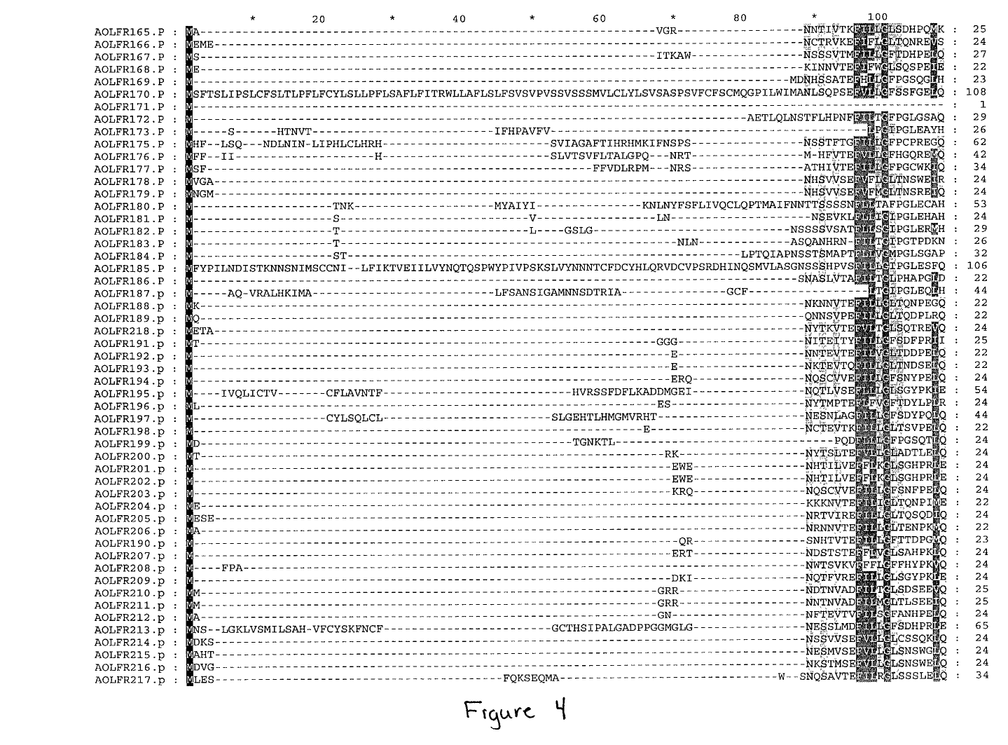

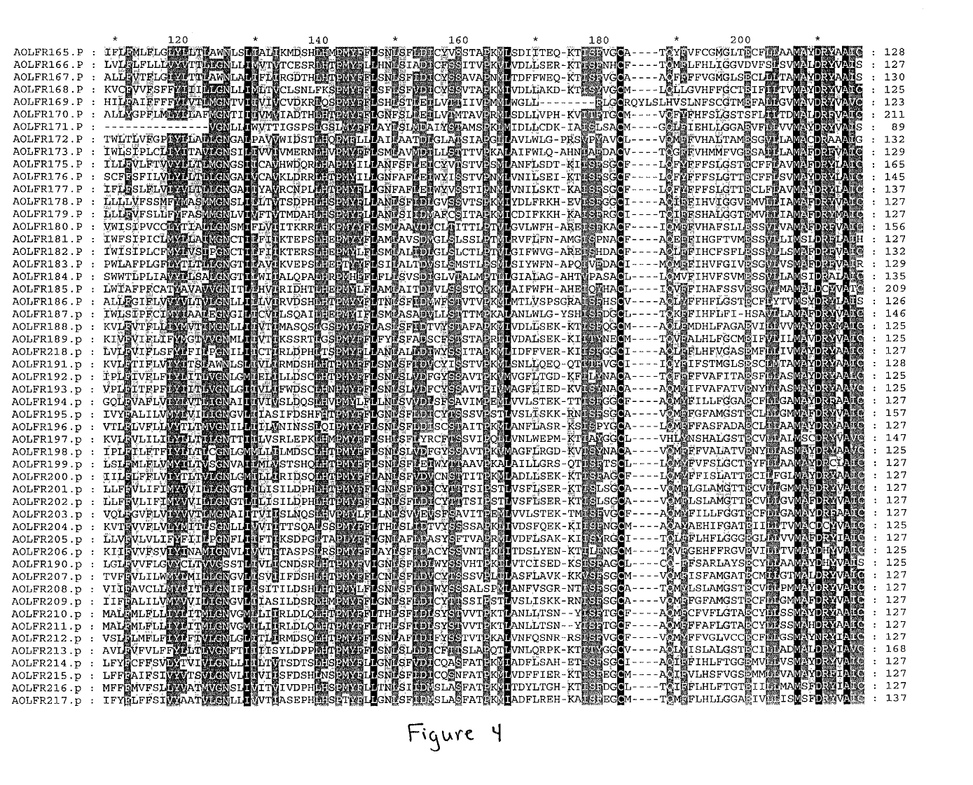

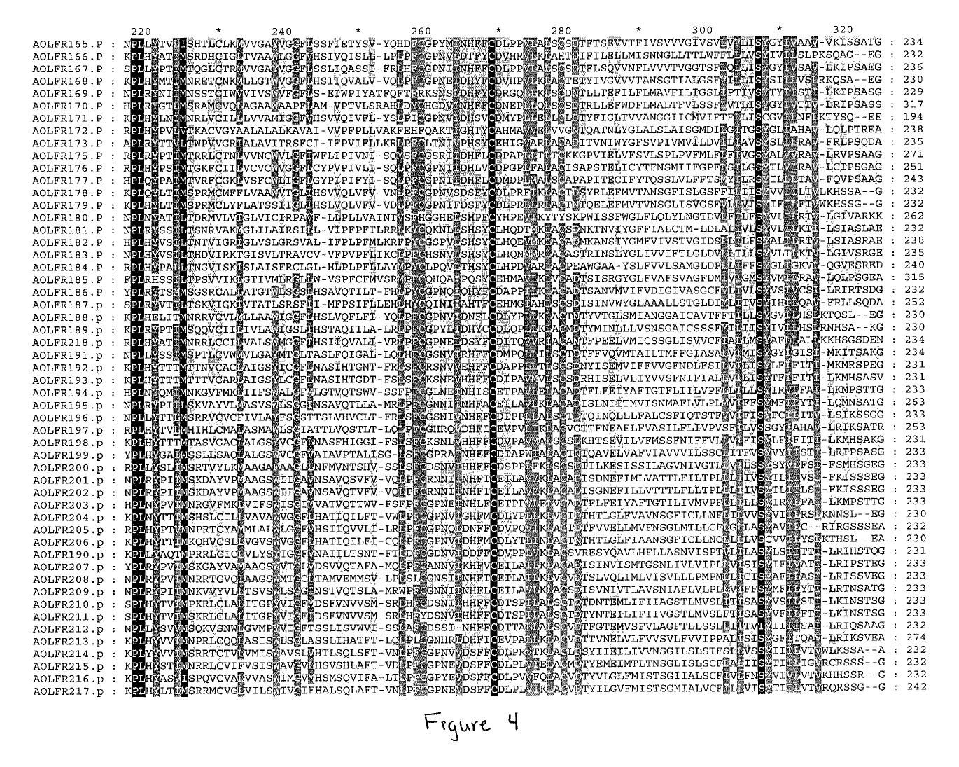

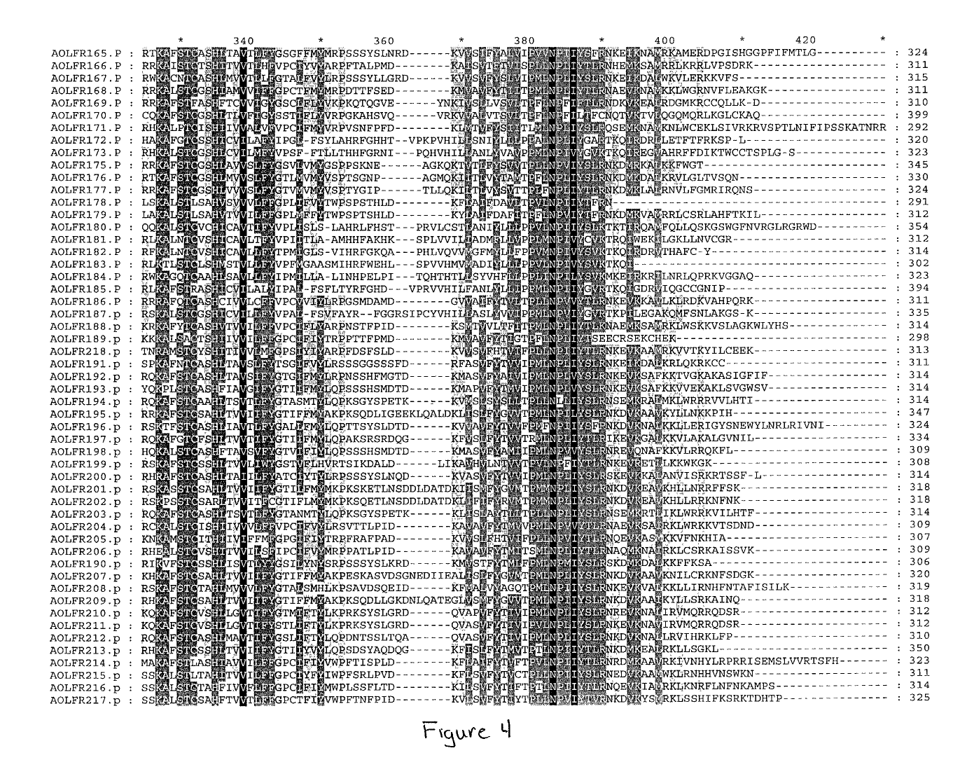

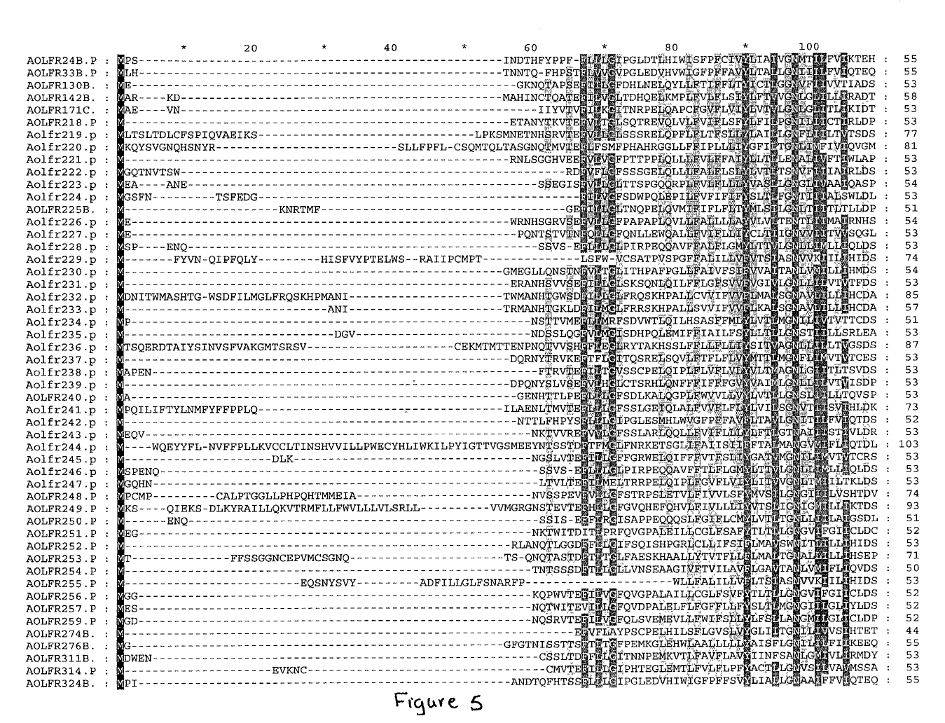

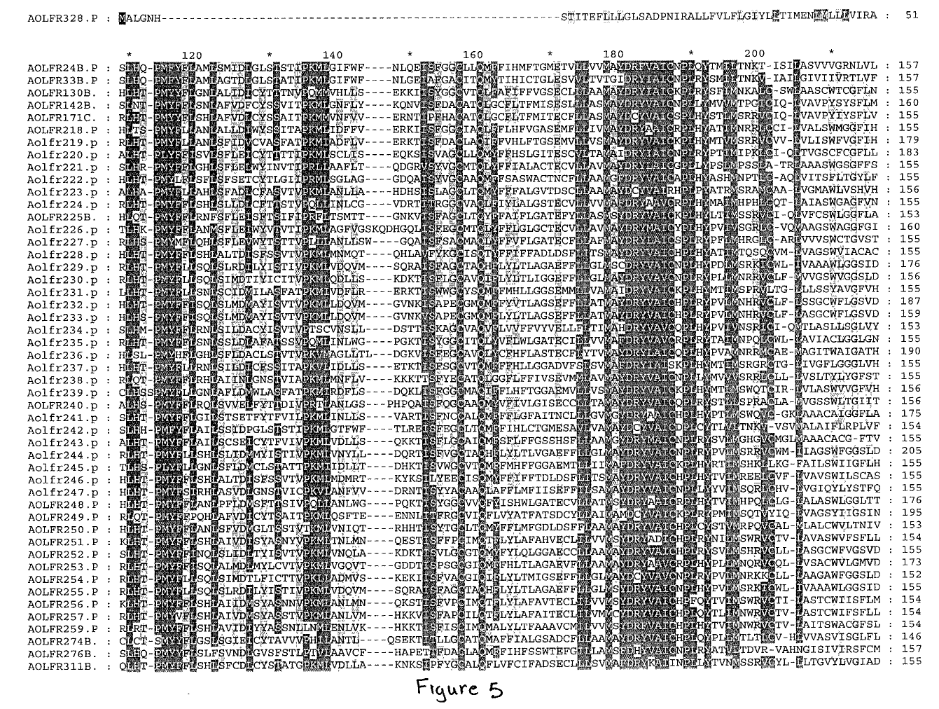

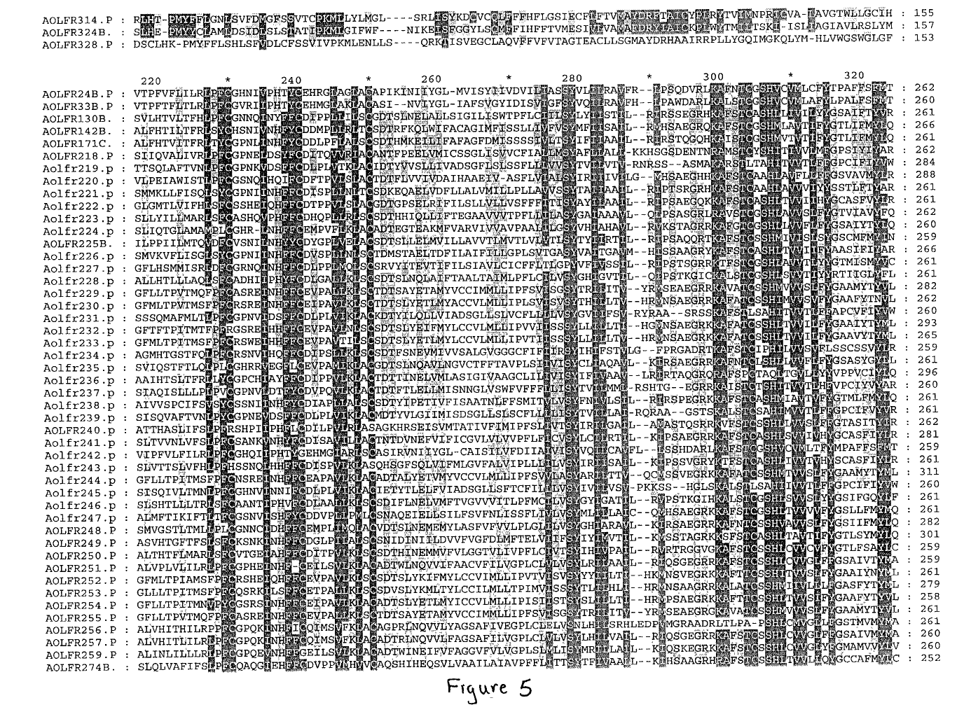

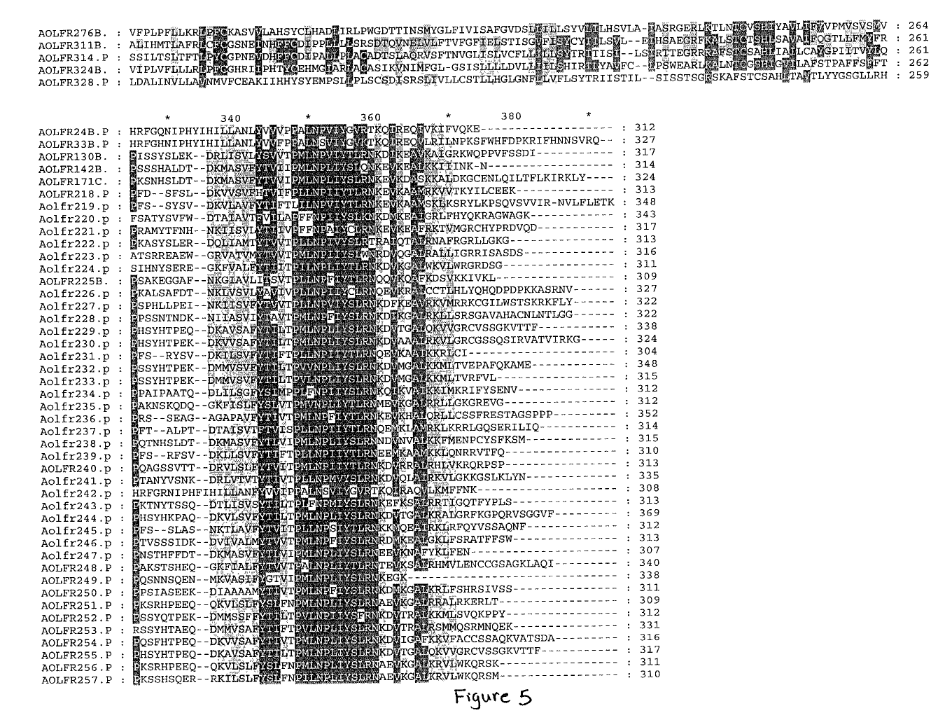

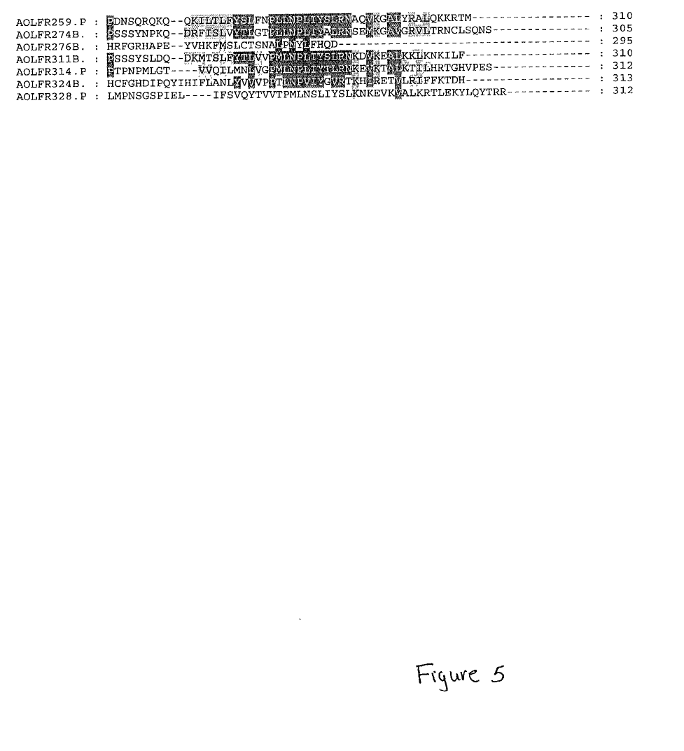

Another example of a useful algorithm is PILEUP. PILEUP creates a multiple sequence alignment from a group of related sequences using progressive, pairwise alignments to show relationship and percent sequence identity. It also plots a so-called “tree” or “dendogram” showing the clustering relationships used to create the alignment (see, e.g., FIG. 2). PILEUP uses a simplification of the progressive alignment method of Feng & Doolittle, J Mol. Evol. 35:351-60 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984) encoded by the genes were derived by conceptual translation of the corresponding open reading frames. Comparison of these protein sequences to all known proteins in the public sequence databases using BLASTP algorithm revealed their strong homology to the members of the mammalian olfactory receptor family, each of the odorant receptor sequences having at least 50%, and preferably at least 55%, at least 60%, at least 65%, and most preferably at least 70%, amino acid identity to at least one known member of the family.

The nucleic acid molecules of the present invention are typically intronless and encode putative OR proteins generally having lengths of approximately 290 to approximately 400 amino acid residues that contain seven transmembrane domains, as predicted by hydrophobicity plotting analysis, indicating that they belong to the G protein-coupled receptor 7-transmembrane (7TM) superfamily, which includes the subset of taste and olfactory receptors. In addition to the overall structural similarity, each of the ORs identified herein has a characteristic sequence signature of an olfactory receptor. In particular, all the identified sequences contain very close matches to the following consensus amino acid motifs (Mombaerts, 1999, Pilpel 1999): EFILL (SEQ ID NO: 513) before transmembrane domain 1, LHTPMY (SEQ ID NO: 514) in intracellular loop 1, MAYDRYVAIC (SEQ ID NO: 510) at the end of transmembrane domain 3 and the beginning of intracellular loop 2, SY at the end of transmembrane domain 5, FSTCSSH (SEQ ID NO: 516) in the beginning of transmembrane domain 6, and PMLNPF (SEQ ID NO: 517) in transmembrane domain 7. Combination of all the above-mentioned structural features of the identified genes and encoded proteins strongly suggests that they represent novel members of the human olfactory receptor family.

As noted above, complete or partial sequences of numerous human and other eukaryotic olfactory receptors are currently known. The novel human receptors have amino acid sequences distinctly different from the previously known human olfactory receptors, which suggests their different specificity in odorant recognition. Therefore, these novel receptors and their genes can be used, alone or in combination with known olfactory receptors, in developing detection systems and assays for chemically distinct types of odorants not recognized by the known receptors, as well as for diagnostic and research purposes.

B. Definitions

As used herein, the following terms have the meanings ascribed to them unless specified otherwise.

“OR” refers to one or more members of a family of G protein-coupled receptors that are expressed in olfactory cells. Olfactory receptor cells can also be identified on the basis of morphology (see, e.g., Roper, supra), or by the expression of proteins specifically expressed in olfactory cells. OR family members may have the ability to act as receptors for olfactory transduction.

“OR” nucleic acids encode a family of GPCRs with seven transmembrane regions that have “G protein-coupled receptor activity,” e.g., they may bind to G proteins in response to extracellular stimuli and promote production of second messengers such as IP3, cAMP, cGMP, and Ca²⁺ via stimulation of enzymes such as phospholipase C and adenylate cyclase (for a description of the structure and function of GPCRs, see, e.g., Fong, supra, and Baldwin, supra). A single olfactory cell may contain many distinct OR polypeptides.

Topologically, certain chemosensory GPCRs have an “N-terminal domain;” “extracellular domains;” “transmembrane domains” comprising seven transmembrane regions, and corresponding cytoplasmic, and extracellular loops; “cytoplasmic domains,” and a “C-terminal domain” (see, e.g., Hoon et al., Cell, 96:541-51 (1999); Buck & Axel, Cell, 65:175-87 (1991)). These domains can be structurally identified using methods known to those of skill in the art, such as sequence analysis programs that identify hydrophobic and hydrophilic domains (see, e.g., Stryer, Biochemistry, (3rd ed. 1988); see also any of a number of Internet based sequence analysis programs, such as those found at dot.imgen.bcm.tmc.edu). Such domains are useful for making chimeric proteins and for in vitro assays of the invention, e.g., ligand binding assays.

“Extracellular domains” therefore refers to the domains of OR polypeptides that protrude from the cellular membrane and are exposed to the extracellular face of the cell. Such domains generally include the “N terminal domain” that is exposed to the extracellular face of the cell, and optionally can include portions of the extracellular loops of the transmembrane domain that are exposed to the extracellular face of the cell, i.e., the loops between transmembrane regions 2 and 3, between transmembrane regions 4 and 5, and between transmembrane regions 6 and 7.

The “N terminal domain” region starts at the N-terminus and extends to a region close to the start of the transmembrane domain. “Transmembrane domain,” which comprises the seven “transmembrane regions,” refers to the domain of OR polypeptides that lies within the plasma membrane, and may also include the corresponding cytoplasmic (intracellular) and extracellular loops. The seven transmembrane regions and extracellular and cytoplasmic loops can be identified using standard methods, as described in Kyte & Doolittle, J Mol. Biol., 157:105-32 (1982)), or in Stryer, supra. The general secondary and tertiary structure of transmembrane domains, in particular the seven transmembrane domains of 7-transmembrane receptors such as olfactory receptors, are well known in the art. Thus, primary structure sequence can be designed or predicted based on known transmembrane domain sequences, as described in detail below. These transmembrane domains are useful for in vitro ligand-binding assays, both soluble and solid phase.

“Cytoplasmic domains” refers to the domains of OR polypeptides that face the inside of the cell, e.g., the “C terminal domain” and the intracellular loops of the transmembrane domain, e.g., the intracellular loop between transmembrane regions 1 and 2, the intracellular loop between transmembrane regions 3 and 4, and the intracellular loop between transmembrane regions 5 and 6. “C terminal domain” refers to the region that spans the end of the last transmembrane domain and the C-terminus of the protein, and which is normally located within the cytoplasm.

The term “ligand-binding region” or “ligand-binding domain” refers to sequences derived from a chemosensory receptor, particularly an olfactory receptor, that substantially incorporates at least transmembrane domains II to VII. The ligand-binding region may be capable of binding a ligand, and more particularly, an odorant.

The phrase “functional effects” in the context of assays for testing compounds that modulate OR family member mediated olfactory transduction includes the determination of any parameter that is indirectly or directly under the influence of the receptor, e.g., functional, physical and chemical effects. It includes ligand binding, changes in ion flux, membrane potential, current flow, transcription, G protein binding, GPCR phosphorylation or dephosphorylation, signal transduction, receptor-ligand interactions, second messenger concentrations (e.g., cAMP, cGMP, IP3, or intracellular Ca²⁺ ), in vitro, in vivo, and ex vivo and also includes other physiologic effects such increases or decreases of neurotransmitter or hormone release.

By “determining the functional effect” in the context of assays is meant assays for a compound that increases or decreases a parameter that is indirectly or directly under the influence of an OR family member, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte OR gene expression; tissue culture cell OR expression; transcriptional activation of OR genes; ligand-binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

“Inhibitors,” “activators,” and “modulators” of OR genes or proteins are used interchangeably to refer to inhibitory, activating, or modulating molecules identified using in vitro and in vivo assays for olfactory transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate olfactory transduction, e.g., antagonists. Activators are compounds that, e.g., bind to, stimulate, increase, open, activate, facilitate, enhance activation, sensitize, or up regulate olfactory transduction, e.g., agonists. Modulators include compounds that, e.g., alter the interaction of a receptor with: extracellular proteins that bind activators or inhibitor (e.g., ebnerin and other members of the hydrophobic carrier family); G proteins; kinases (e.g., homologs of rhodopsin kinase and beta adrenergic receptor kinases that are involved in deactivation and desensitization of a receptor); and arresting, which also deactivate and desensitize receptors. Modulators can include genetically modified versions of OR family members, e.g., with altered activity, as well as naturally occurring and synthetic ligands, antagonists, agonists, small chemical molecules and the like. Such assays for inhibitors and activators include, e.g., expressing OR family members in cells or cell membranes, applying putative modulator compounds, in the presence or absence of tastants, e.g., sweet tastants, and then determining the functional effects on olfactory transduction, as described above. Samples or assays comprising OR family members that are treated with a potential activator, inhibitor, or modulator are compared to control samples without the inhibitor, activator, or modulator to examine the extent of modulation. Control samples (untreated with modulators) are assigned a relative OR activity value of 100%. Inhibition of a OR is achieved when the OR activity value relative to the control is about 80%, optionally 50% or 25-0%. Activation of an OR is achieved when the OR activity value relative to the control is 110%, optionally 150%, optionally 200-500%, or 1000-3000% higher.

The terms “purified,” “substantially purified,” and “isolated” as used herein refer to the state of being free of other, dissimilar compounds with which the compound of the invention is normally associated in its natural state, so that the “purified,” “substantially purified,” and “isolated” subject comprises at least 0.5%, 1%, 5%, 10%, or 20%, and most preferably at least 50% or 75% of the mass, by weight, of a given sample. In one preferred embodiment, these terms refer to the compound of the invention comprising at least 95% of the mass, by weight, of a given sample. As used herein, the terms “purified,” “substantially purified,” and “isolated” “isolated,” when referring to a nucleic acid or protein, of nucleic acids or proteins, also refers to a state of purification or concentration different than that which occurs naturally in the mammalian, especially human, body. Any degree of purification or concentration greater than that which occurs naturally in the mammalian, especially human, body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

As used herein, the term “isolated,” when referring to a nucleic acid or polypeptide refers to a state of purification or concentration different than that which occurs naturally in the mammalian, especially human, body. Any degree of purification or concentration greater than that which occurs naturally in the body, including (1) the purification from other naturally-occurring associated structures or compounds, or (2) the association with structures or compounds to which it is not normally associated in the body are within the meaning of “isolated” as used herein. The nucleic acids or polypeptides described herein may be isolated or otherwise associated with structures or compounds to which they are not normally associated in nature, according to a variety of methods and processed known to those of skill in the art.

As used herein, the terms “amplifying” and “amplification” refer to the use of any suitable amplification methodology for generating or detecting recombinant or naturally expressed nucleic acid, as described in detail, below. For example, the invention provides methods and reagents (e.g., specific degenerate oligonucleotide primer pairs) for amplifying (e.g., by polymerase chain reaction, PCR) naturally expressed (e.g., genomic or mRNA) or recombinant (e.g., cDNA) nucleic acids of the invention (e.g., tastant-binding sequences of the invention) in vivo or in vitro.

The term “7-transmembrane receptor” means a polypeptide belonging to a superfamily of transmembrane proteins that have seven domains that span the plasma membrane seven times (thus, the seven domains are called “transmembrane” or “TM” domains TM I to TM VII). The families of olfactory and certain taste receptors each belong to this super-family. 7-transmembrane receptor polypeptides have similar and characteristic primary, secondary and tertiary structures, as discussed in further detail below.

The term “library” means a preparation that is a mixture of different nucleic acid or polypeptide molecules, such as the library of recombinantly generated chemosensory, particularly olfactory receptor ligand-binding domains generated by amplification of nucleic acid with degenerate primer pairs, or an isolated collection of vectors that incorporate the amplified ligand-binding domains, or a mixture of cells each randomly transfected with at least one vector encoding an olfactory receptor.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones (see e.g., Oligonucleotides and Analogues, a Practical Approach, ed. F. Eckstein, Oxford Univ. Press (1991); Antisense Strategies, Annals of the N.Y. Acad. of Sci., Vol. 600, Eds. Baserga et al. (NYAS 1992); Milligan J Med. Chem. 36:1923-1937 (1993); Antisense Research and Applications (1993, CRC Press), WO 97/03211; WO 96/39154; Mata, Toxicol. Appl. Pharmacol. 144:189-197 (1997); Strauss-Soukup, Biochemistry 36:8692-8698 (1997); Samstag, Antisense Nucleic Acid Drug Dev, 6:153-156 (1996)).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues (Batzer et al., Nucleic Acid Res., 19:5081 (1991); Ohtsuka et al., J Biol. Chem., 260:2605-08 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “plasma membrane translocation domain” or simply “translocation domain” means a polypeptide domain that, when incorporated into the amino terminus of a polypeptide coding sequence, can with great efficiency “chaperone” or “translocate” the hybrid (“fusion”) protein to the cell plasma membrane. For instance, a “translocation domain” may be derived from the amino terminus of the bovine rhodopsin receptor polypeptide. In one embodiment, the translocation domain may be functionally equivalent to an exemplary translocation domain (5′-MNGTEGPNFYVPFSNKTGVV; SEQ ID NO: 518). However, rhodopsin from any mammal may be used, as can other translocation facilitating sequences. Thus, the translocation domain is particularly efficient in translocating 7-transmembrane fusion proteins to the plasma membrane, and a protein (e.g., an olfactory receptor polypeptide) comprising an amino terminal translocating domain will be transported to the plasma membrane more efficiently than without the domain. However, if the N-terminal domain of the polypeptide is active in binding, the use of other translocation domains may be preferred.

“Functional equivalency” means the domain's ability and efficiency in translocating newly translated proteins to the plasma membrane as efficiently as exemplary SEQ ID NO: 518 under similar conditions; relatively efficiencies an be measured (in quantitative terms) and compared, as described herein. Domains falling within the scope of the invention can be determined by routine screening for their efficiency in translocating newly synthesized polypeptides to the plasma membrane in a cell (mammalian, Xenopus, and the like) with the same efficiency as the twenty amino acid long translocation domain SEQ ID NO: 518, as described in detail below.

The “translocation domain,” “ligand-binding domain”, and chimeric receptors compositions described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides, e.g., translocation domains, ligand-binding domains, or chimeric receptors of the invention. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄), thiazole, retroamide, thioamide, or ester (see, e.g., Spatola, Chemistry and Biochemistry of Amino Acids, Peptides and Proteins, 7:267-357, “Peptide Backbone Modifications,” Marcell Dekker, N.Y. (1983)). A polypeptide can also be characterized as a mimetic by containing all or some non-natural residues in place of naturally occurring amino acid residues; non-natural residues are well described in the scientific and patent literature.

A “label” or a “detectable moiety” is a composition detectable by spectroscopic, photochemical, biochemical, immunochemical, or chemical means. For example, useful labels include ³²p, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridisation with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5× SSC, and 1% SDS, incubating at 42° C., or, 5× SSC, 1% SDS, incubating at 65° C., with wash in 0.2× SSC, and 0.1% SDS at 65° C. Such hybridizations and wash steps can be carried out for, e.g., 1, 2, 5, 10, 15, 30, 60; or more minutes.

Nucleic acids that do not hybridize to each other under stringent conditions are still substantially related if the polypeptides that they encode are substantially related. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chains respectively.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-OR” antibody is an antibody or antibody fragment that specifically binds a polypeptide encoded by a OR gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or, “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to an OR family member from specific species such as rat, mouse, or human can be selected to obtain only those polyclonal antibodies that are specifically immunoreactive with the OR polypeptide or an immunogenic portion thereof and not with other proteins, except for orthologs or polymorphic variants and alleles of the OR polypeptide. This selection may be achieved by subtracting out antibodies that cross-react with OR molecules from other species or other OR molecules. Antibodies can also be selected that recognize only OR GPCR family members but not GPCRs from other families. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual, (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

The term “expression vector” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes which contain only the minimum elements needed for transcription of the recombinant nucleic acid.

By “host cell” is meant a cell that contains an expression vector and supports the replication or expression of the expression vector. Host cells may be prokaryotic cells such as E. toll, or eukaryotic cells such as yeast, insect, amphibian, or mammalian cells such as CHO, HeLa, HEK-293, and the like, e.g., cultured cells, explants, and cells in vivo.

C. Isolation and Expression of Olfactory Receptors

Isolation and expression of the ORs, or fragments or variants thereof, of the invention can be performed as described below. PCR primers can be used for the amplification of nucleic acids encoding olfactory receptor ligand-binding regions and libraries of these nucleic acids can thereby be generated. Libraries of expression vectors can then be used to infect or transfect host cells for the functional expression of these libraries. These genes and vectors can be made and expressed in vitro or in vivo. One of skill will recognize that desired phenotypes for altering and controlling nucleic acid expression can be obtained by modulating the expression or activity of the genes and nucleic acids (e.g., promoters, enhancers and the like) within the vectors of the invention. Any of the known methods described for increasing or decreasing expression or activity can be used. The invention can be practiced in conjunction with any method or protocol known in the art, which are well described in the scientific and patent literature.

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to mammalian cells, e.g., bacterial, yeast, insect or plant systems.

Alternatively, these nucleic acids can be synthesized in vitro by well-known chemical synthesis techniques, as described in, e.g., Carruthers, Cold Spring Harbor Symp. Quant. Biol. 47:411-418 (1982); Adams, Am. Chem. Soc. 105:661 (1983); Belousov, Nucleic Acids Res. 25:3440-3444 (1997); Frenkel, Free Radic. Biol. Med. 19:373-380 (1995); Blommers, Biochemistry 33:7886-7896 (1994); Narang, Meth. Enzymol. 68:90 (1979); Brown, Meth. Enzymol. 68:109 (1979); Beaucage, Tetra. Lett. 22:1859 (1981); U.S. Pat. No. 4,458,066. Double-stranded DNA fragments may then be obtained either by synthesizing the complementary strand and annealing the strands together under appropriate conditions, or by adding the complementary strand using DNA polymerase with an appropriate primer sequence.

Techniques for the manipulation of nucleic acids, such as, for example, for generating mutations in sequences, subcloning, labeling probes, sequencing, hybridization and the like are well described in the scientific and patent literature. See, e.g., Sambrook, ed., Molecular Cloning: a Laboratory manual (2nd ed.), Vols. 1-3, Cold Spring Harbor Laboratory (1989); Current Protocols in Molecular Biology, Ausubel, ed. John Wiley & Sons, Inc., New York (1997); Laboratory Techniques in Biochemistry and Molecular Biology: Hybridization With Nucleic Acid Probes, Part I, Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immunoelectrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Oligonucleotide primers are used to amplify nucleic acid encoding an olfactory receptor ligand-binding region. The nucleic acids described herein can also be cloned or measured quantitatively using amplification techniques. Using exemplary degenerate primer pair sequences, (see below), the skilled artisan can select and design suitable oligonucleotide amplification primers. Amplification methods are also well known in the art, and include, e.g., polymerase chain reaction, PCR (PCR Protocols, a Guide to Methods and Applications, ed. Innis. Academic Press, N.Y. (1990) and PCR Strategies, ed. Innis, Academic Press, Inc., N.Y. (1995), ligase chain reaction (LCR) (see, e.g., Wu, Genomics 4:560 (1989); Landegren, Science 241:1077,(1988); Barringer, Gene 89:117 (1990)); transcription amplification (see, e.g., Kwoh, PNAS, 86:1173 (1989)); and, self-sustained sequence replication (see, e.g., Guatelli, PNAS, 87:1874 (1990)); Q Beta replicase amplification (see, e.g., Smith, J Clin. Microbiol. 35:1477-1491 (1997)); automated Q-beta replicase amplification assay (see, e.g., Burg, Mol. Cell. Probes 10:257-271 (1996)) and other RNA polymerase mediated techniques (e.g., NASBA, Cangene, Mississauga, Ontario); see also Berger, Methods Enzymol. 152:307-316 (1987); Sambrook; Ausubel; U.S. Pat. Nos. 4,683,195 and 4,683,202; Sooknanan, Biotechnology 13:563-564 (1995).

Once amplified, the nucleic acids, either individually or as libraries, may be cloned according to methods known in the art, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” the PCR primer pair. For example, Pst I and Bsp E1 sites were designed into the exemplary primer pairs of the invention. These particular restriction sites have a sequence that, when ligated, are “in-frame” with respect to the 7-membrane receptor “donor” coding sequence into which they are spliced (the ligand-binding region coding sequence is internal to the 7-membrane polypeptide, thus, if it is desired that the construct be translated downstream of a restriction enzyme splice site, out of frame results should be avoided; this may not be necessary if the inserted ligand-binding domain comprises substantially most of the transmembrane VII region). The primers can be designed to retain the original sequence of the “donor” 7-membrane receptor (the Pst I and Bsp E1 sequence in he primers of the invention generate an insert that, when ligated into the Pst I/Bsp E1 cut vector, encode residues found in the “donor” mouse olfactory receptor M4 sequence). Alternatively, the primers can encode amino acid residues that are conservative substitutions (e.g., hydrophobic for hydrophobic residue, see above discussion) or functionally benign substitutions (e.g., do not prevent plasma membrane insertion, cause cleavage by peptidase, cause abnormal folding of receptor, and the like).

The primer pairs are designed to selectively amplify ligand-binding regions of olfactory receptor proteins. These domain regions may vary for different ligands, and more particularly odorants; thus, what may be a minimal binding region for one ligand, and more particularly odorants, may be too limiting for a second potential ligand. Thus, domain regions of different sizes comprising different domain structures may be amplified; for example, transmembrane (TM) domains II through VII, III through VII, III through VI or II through VI, or variations thereof (e.g., only a subsequence of a particular domain, mixing the order of the domains, and the like), of a 7-transmembrane OR.

As domain structures and sequence of many 7-membrane proteins, particularly olfactory receptors, are known, the skilled artisan can readily select domain-flanking and internal domain sequences as model sequences to design degenerate amplification primer pairs. For example, a nucleic acid sequence encoding domain regions II through VII can be generated by PCR amplification using a primer pair. To amplify a nucleic acid comprising transmembrane domain I (TM I) sequence, a degenerate primer can be designed from a nucleic acid that encodes the amino acid sequence LFLLYL3′ (SEQ ID NO: 519). Such a degenerate primer can be used to generate a binding domain incorporating TM I through TM III, TM I through TM IV, TM I through TM V, TM I through TM VI or TM I through TM VII).

To amplify a nucleic acid comprising a transmembrane domain III (TM III) sequence, a degenerate primer (of at least about 17 residues) can be designed from a nucleic acid that encodes the amino acid sequence M(A/G)(Y/F)DRYVAI 3′ (SEQ ID NO: 520) (encoded by a nucleic acid sequence such as 5′-ATGG(G/C)CT(A/T)TGACCG(C/A/T)T(AT)(C/T)GT-3′ (SEQ ID NO: 521)). Such a degenerate primer can be used to generate a binding domain incorporating TM III through TM IV, TM II through TM V, TM III through TM VI or TM III through TM VII.

To amplify transmembrane domain VI (TM VI) sequence, a degenerate primer (of at least about 17 residues) can be designed from nucleic acid encoding an amino acid sequence TC(G/A)SHL (SEQ ID NO: 522), encoded by a sequence such as 5′-AG(G/A)TGN(G/C)(T/A)N(G/C)C(G/A)CANGT-3′) 3′ (SEQ ID NO: 522). Such a degenerate primer can be used to generate a binding domain incorporating TM I through TM VI, TM II through TM VI, TM III through TM VI or TM IV through TM VI).

Paradigms to design degenerate primer pairs are well known in the art. For example, a COnsensus-DEgenerate Hybrid Oligonucleotide Primer (CODEHOP) (SEQ ID NO: 523) strategy computer program is accessible as http://blocks.fhcrc.org/codehop.html, and is directly linked from the BlockMaker multiple sequence alignment site for hybrid primer prediction beginning with a set of related protein sequences, as known olfactory receptor ligand-binding regions (see, e.g., Rose, Nucleic Acids Res. 26:1628-1635 (1998); Singh, Biotechniques, 24:318-19 (1998)).

Means to synthesize oligonucleotide primer pairs are well known in the art. “Natural” base pairs or synthetic base pairs can be used. For example, use of artificial nucleobases offers a versatile approach to manipulate primer sequence and generate a more complex mixture of amplification products. Various families of artificial nucleobases are capable of assuming multiple hydrogen bonding orientations through internal bond rotations to provide a means for degenerate molecular recognition. Incorporation of these analogs into a single position of a PCR primer allows for generation of a complex library of amplification products. See, e.g., Hoops, Nucleic Acids Res. 25:4866-4871 (1997). Nonpolar molecules can also be used to mimic the shape of natural DNA bases. A non-hydrogen-bonding shape mimic for adenine can replicate efficiently and selectively against a nonpolar shape mimic for thymine (see, e.g., Morales, Nat. Struct. Biol. 5:950-954 (1998)). For example, two degenerate bases can be the pyrimidine base 6H, 8H-3,4-dihydropyrimido[4,5-c][1,2]oxazin-7-one or the purine base N6-methoxy-2,6-diaminopurine (see, e.g., Hill, PNAS, 95:4258-63 (1998)). Exemplary degenerate primers of the invention incorporate the nucleobase analog 5′-Dimethoxytrityl-N-benzoyl-2′-deoxy-Cytidine,3′-[(2-cyanoethyl)-(N,N-diisopropyl)]-phosphoramidite (the term “P” in the sequences, see above). This pyrimidine analog hydrogen bonds with purines, including A and G residues.

Exemplary primer pairs for amplification of olfactory receptor transmembrane domains II through VII include:

(a) 5′-GGGGTCCGGAG(A/G)(C/G)(A/G)TA(A/G/T)AT(A/G/P)A(A/G/P)(A/G/P)GG-3′ (SEQ ID NO: 524) and

5′-GGGGCTGCAGACACC(A/C/G/T)ATGTA(C/T)(C/T)T(A/C/G/T)TT(C/T) (C/T)T-3′ (SEQ ID NO: 525).

(b) 5′-GGGGTCCGGAG(A/G)(C/G)T(A/G)A(A/GI/T)AT(A/G/P)A(A/G/P)(A/G/P)GG-3′ (SEQ ID NO: 526); and

5′-GGGGCTGCAGACACC(AC/G/T)ATGTA(C/T)(C/T)T(A/C/G/T)TT(C/T) (C/T)T-3′ (SEQ ID NO: 527)

(c) 5′-GGGGTCCGGAG(A/G)(C/G)T(A/G)A(A/G/T)AT(A/G/C/T)A(A/G/C/T) (A/G/C/T)GG-3′ (SEQ ID NO: 528) and

5′-GGGGCTGCAGACACC(A/C/G/T)ATGTA(C/T)(C/T)T(A/C/G/T)TT(C/T) (C/T)T-3′ (SEQ ID NO: 558)

Nucleic acids that encode ligand-binding regions of olfactory receptors may be generated by amplification (e.g., PCR) of appropriate nucleic acid sequences using degenerate primer pairs. The amplified nucleic acid can be genomic DNA from any cell or tissue or mRNA or cDNA derived from olfactory receptor-expressing cells, e.g., olfactory neurons or olfactory epithelium.

Isolation from olfactory receptor-expressing cells is well known in the art (cells expressing naturally or inducibly expressing olfactory receptors can be used to express the hybrid olfactory receptors of the invention to screen for potential odorants and odorant effect on cell physiology, as described below). For example, cells can be identified by olfactory marker protein (OMP), an abundant cytoplasmic protein expressed almost exclusively in mature olfactory sensory neurons (see, e.g., Buiakova, PNAS, 93:9858-63 (1996)). Shirley, Eur. J. Biochem. 32:485-494 (1983), describes a rat olfactory preparation suitable for biochemical studies in vitro on olfactory mechanisms. Cultures of adult rat olfactory receptor neurons are described by Vargas, Chem. Senses 24:211-216 (1999). Because these cultured neurons exhibit typical voltage-gated currents and are responsive to application of odorants, they can also be used to express the hybrid olfactory receptors of the invention for odorant screening (endogenous olfactory receptor can be initially blocked, if desired, by, e.g., antisense, knockout, and the like). U.S. Pat. No. 5,869,266 describes culturing human olfactory neurons for neurotoxicity tests and screening. Murrell, J. Neurosci. 19:8260-8270 (1999), describes differentiated olfactory receptor-expressing cells in culture that respond to odorants, as measured by an influx of calcium.

In one embodiment, hybrid protein-coding sequences comprising nucleic acids ORs fused to the translocation sequences described herein may be constructed. Also provided are hybrid ORs comprising the translocation motifs and ligand-binding domains of olfactory receptors. These nucleic acid sequences can be operably linked to transcriptional or translational control elements, e.g., transcription and translation initiation sequences, promoters and enhancers, transcription and translation terminators, polyadenylation sequences, and other sequences useful for transcribing DNA into RNA. In construction of recombinant expression cassettes, vectors, transgenics, and a promoter fragment can be employed to direct expression of the desired nucleic acid in all tissues. Olfactory cell-specific transcriptional elements can also be used to express the fusion polypeptide receptor, including, e.g., a 6.7 kb region upstream of the M4 olfactory receptor coding region. This region was sufficient to direct expression in olfactory epithelium with wild type zonal restriction and distributed neuronal expression for endogenous olfactory receptors (Qasba, J. Neurosci. 18:227-236 (1998)). Receptor genes are normally expressed in a small subset of neurons throughout a zonally restricted region of the sensory epithelium. The transcriptional or translational control elements can be isolated from natural sources, obtained from such sources as ATCC or GenBank libraries, or prepared by synthetic or recombinant methods.

In another embodiment, fusion proteins, either having C-terminal or, more preferably, N-terminal translocation sequences, may also comprise the translocation motif described herein. However, these fusion proteins can also comprise additional elements for, e.g., protein detection, purification, or other applications. Detection and purification facilitating domains include, e.g., metal chelating peptides such as polyhistidine tracts or histidine-tryptophan modules or other domains that allow purification on immobilized metals; maltose binding protein; protein A domains that allow purification on immobilized immunoglobulin; or the domain utilized in the FLAGS extension/affinity purification system (Immunex Corp, Seattle Wash.).

The inclusion of a cleavable linker sequences such as Factor Xa (see, e.g., Ottavi, Biochimie 80:289-293 (1998)), subtilisin protease recognition motif (see, e.g., Polyak, Protein Eng. 10:615-619 (1997)); enterokinase (Invitrogen, San Diego, Calif.), and the like, between the translocation domain (for efficient plasma membrane expression) and the rest of the newly translated polypeptide may be useful to facilitate purification. For example, one construct can include a polypeptide-encoding nucleic acid sequence linked to six histidine residues followed by a thioredoxin, an enterokinase cleavage site (see, e.g., Williams, Biochemistry 34:1787-1797 (1995)), and an amino terminal translocation domain. The histidine residues facilitate detection and purification while the enterokinase cleavage site provides a means for purifying the desired protein(s) from the remainder of the fusion protein. Technology pertaining to vectors encoding fusion proteins and application of fusion proteins are well described in the scientific and patent literature (see, e.g., Kroll, DNA Cell. Biol. 12:441-53 (1993)).

Expression vectors, either as individual expression vectors or as libraries of expression vectors, comprising the olfactory binding domain-encoding sequences may be introduced into a genome or into the cytoplasm or a nucleus of a cell and expressed by a variety of conventional techniques, well described in the scientific and patent literature (see, e.g., Roberts, Nature 328:731 (1987); Berger supra; Schneider, Protein Expr. Purif. 6435:10 (1995); Sambrook; Tijssen; Ausubel). Product information from manufacturers of biological reagents and experimental equipment also provide information regarding known biological methods. The vectors can be isolated from natural sources, obtained from such sources as ATCC or GenBank libraries, or prepared by synthetic or recombinant methods.

The nucleic acids can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required. For example, the marker may encode antibiotic resistance (e.g., chloramphenicol, kanamycin, G418, bleomycin, hygromycin) or herbicide resistance (e.g., chlorosulfron or Basta) to permit selection of those cells transformed with the desired DNA sequences (see, e.g., Blondelet-Rouault, Gene 190:315-17 (1997); Aubrecht, J. Pharmacol. Exp. Ther., 281:992-97 (1997)). Because selectable marker genes conferring resistance to substrates like neomycin or hygromycin can only be utilized in tissue culture, chemoresistance genes are also used as selectable markers in vitro and in vivo.

A chimeric nucleic acid sequence may encode a ligand-binding domain within any 7-transmembrane polypeptide. 7-transmembrane receptors belong to a superfamily of transmembrane (TM) proteins having seven domains that traverse a plasma membrane seven times. Each of the seven domains spans the plasma membrane (TM I to TM VII). Because 7-transmembrane receptor polypeptides have similar primary sequences and secondary and tertiary structures, structural domains (e.g., TM domains) can be readily identified by sequence analysis. For example, homology modeling, Fourier analysis and helical periodicity detection can identify and characterize the seven domains with a 7-transmembrane receptor sequence. Fast Fourier Transform (FFT) algorithms can be used to assess the dominant periods that characterize profiles of the hydrophobicity and variability of analyzed sequences. To predict TM domains and their boundaries and topology, a “neural network algorithm” by “PHD server” can be used, as done by Pilpel, Protein Science 8:969-977 (1999); Rost, Protein Sci. 4:521-533 (1995). Periodicity detection enhancement and alpha helical periodicity index can be done as by, e.g., Donnelly, Protein Sci. 2:55-70 (1993). Other alignment and modeling algorithms are well known in the art, see, e.g., Peitsch, Receptors Channels 4:161-164 (1996); Cronet, Protein Eng. 6:59-64 (1993) (homology and “discover modeling”); http://bioinfo.weizmann.ac.il/.

The library sequences include receptor sequences that correspond to TM ligand-binding domains, including, e.g., TM II to VII, TM II to VI, TM III to VII, and TM III to VII, that have been amplified (e.g., PCR) from mRNA of or cDNA derived from, e.g., olfactory receptor-expressing neurons or genomic DNA.

Libraries of olfactory receptor ligand-binding TM domain sequences can include a various TM domains or variations thereof, as described above. These sequences can be derived from any 7-transmembrane receptor. Because these polypeptides have similar primary sequences and secondary and tertiary structures, the seven domains can be identified by various analyses well known in the art, including, e.g., homology modeling, Fourier analysis and helical periodicity (see, e.g., Pilpel supra), as described above. Using this information sequences flanking the seven domains can be identified and used to design degenerate primers for amplification of various combinations of TM regions and subsequences.

The present invention also includes not only the DNA and proteins having the specified amino acid sequences, but also DNA fragments, particularly fragments of, for example, 40, 60, 80, 100, 150, 200, or 250 nucleotides, or more, as well as protein fragments of, for example, 10, 20, 30, 50, 70, 100, or 150 amino acids, or more.

Also contemplated are chimeric proteins, comprising at least 10, 20, 30, 50, 70, 100, or 150 amino acids, or more, of one of at least one of the olfactory receptors described herein, coupled to additional amino acids representing all or part of another G protein receptor, preferably a member of the 7TM superfamily. These chimeras can be made from the instant receptors and a G protein receptor described herein, or they can be made by combining two or more of the present proteins. In one preferred embodiment, one portion of the chimera corresponds to and is derived from one or more of the domains of the seven transmembrane protein described herein, and the remaining portion or portions come from another G protein-coupled receptor. Chimeric receptors are well known in the art, and the techniques for creating them and the selection and boundaries of domains or fragments of G protein-coupled receptors for incorporation therein are also well known. Thus, this knowledge of those skilled in the art can readily be used to create such chimeric receptors. The use of such chimeric receptors can provide, for example, an olfactory selectivity characteristic of one of the receptors specifically disclosed herein, coupled with the signal transduction characteristics of another receptor, such as a well known receptor used in prior art assay systems.

For example, a domain such as a ligand-binding domain, an extracellular domain, a transmembrane domain (e.g., one comprising seven transmembrane regions and corresponding extracellular and cytosolic loops), the transmembrane domain and a cytoplasmic domain, an active site, a subunit association region, etc., can be covalently linked to a heterologous protein. For instance, an extracellular domain can be linked to a heterologous GPCR transmembrane domain, or a heterologous GPCR extracellular domain can be linked to a transmembrane domain. Other heterologous proteins of choice can include, e.g., green fluorescent protein, β-gal, glutamtate receptor, and the rhodopsin presequence.

Polymorphic variants, alleles, and interspecies homologs that are substantially identical to an olfactory receptor disclosed herein can be isolated using the nucleic acid probes described above. It is hypothesized that allelic differences in receptors may explain why there is a difference in olfactory sensation in different human subjects. Accordingly, the identification of such alleles may be significant, especially with respect to producing receptor libraries that adequately represent the olfactory capability of the human population, i.e., which take into account allelic differences in different individuals. Alternatively, expression libraries can be used to clone olfactory receptors and polymorphic variants, alleles, and interspecies homologs thereof, by detecting expressed homologs immunologically with antisera or purified antibodies made against an olfactory polypeptide, which also recognize and selectively bind to the olfactory receptor homolog.

Also within the scope of the invention are host cells for expressing the ORs, fragments, or variants of the invention. To obtain high levels of expression of a cloned gene or nucleic acid, such as cDNAs encoding the olfactory receptors, fragments, or variants of the invention, one of skill typically subclones the nucleic acid sequence of interest into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. However, bacterial or eukaryotic expression systems can be used.

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al.) It is only necessary that the particular genetic engineering procedure used be capable of successfully introducing at lest one gene into the host cell capable of expressing the olfactory receptor, fragment, or variant of interest.

After the expression vector is introduced into the cells, the transfected cells are cultured under conditions favoring expression of the receptor, fragment, or variant of interest, which is then recovered from the culture using standard techniques. Examples of such techniques are well known in the art. See, e.g., WO 00/06593, which is incorporated by reference in a manner consistent with this disclosure.

D. Immunological Detection of OR Polypeptides

In addition to the detection of OR genes and gene expression using nucleic acid hybridization technology, one can also use immunoassays to detect ORs, e.g., to identify olfactory receptor cells, and variants of OR family members. Immunoassays can be used to qualitatively or quantitatively analyze the ORs. A general overview of the applicable technology can be found in Harlow & Lane, Antibodies: A Laboratory Manual (1988).

1. Antibodies to OR Family Members

Methods of producing polyclonal and monoclonal antibodies that react specifically with a OR family member are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature, 256:495-97 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science, 246:1275-81 (1989); Ward et al., Nature, 341:544-46 (1989)).

A number of OR-comprising immunogens may be used to produce antibodies specifically reactive with a OR family member. For example, a recombinant OR protein, or an antigenic fragment thereof, can be isolated as described herein. Suitable antigenic regions include, e.g., the conserved motifs that are used to identify members of the OR family. Recombinant proteins can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. For example, an inbred strain of mice (e.g., BALB/C mice) or rabbits may be immunized with the protein using a standard adjuvant, such as Freund's adjuvant, and a standard immunization protocol. The animal's immune response to the immunogen preparation is monitored by taking test bleeds and determining the titer of reactivity to the OR. When appropriately high titers of antibody to the immunogen are obtained, blood is collected from the animal and antisera are prepared. Further fractionation of the antisera to enrich for antibodies reactive to the protein can be done if desired (see Harlow & Lane, supra).

Monoclonal antibodies may be obtained by various techniques familiar to those skilled in the art. Briefly, spleen cells from an animal immunized with a desired antigen may be immortalized, commonly by fusion with a myeloma cell (see Kohler & Milstein, Eur. J. Immunol., 6:511-19 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse et al., Science, 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 109 or greater are selected and tested for their cross reactivity against non-OR proteins, or even other OR family members or other related proteins from other organisms, using a competitive binding immunoassay. Specific polyclonal antisera and monoclonal antibodies will usually bind with a Kd of at least about 0.1 mM, more usually at least about 1 pM, optionally at least about 0.1 pM or better, and optionally 0.01 pM or better.

Once OR family member specific antibodies are available, individual OR proteins can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

2. Immunological Binding Assays

OR proteins can be detected and/or quantified using any of a number of well recognized immunological binding assays (see, e.g., U.S. Pat. Nos. 4,366,241; 4,376,110; 4,517,288; and 4,837,168). For are view of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case an OR family member or an antigenic subsequence thereof). The antibody (e.g., anti-OR) may be produced by any of a number of means well known to those of skill in the art and as described above.

Immunoassays also often use a labeling agent to specifically bind to and label the complex formed by the antibody and antigen. The labeling agent may itself be one of the moieties comprising the antibody/antigen complex. Thus, the labeling agent may be a labeled OR polypeptide or a labeled anti-OR antibody. Alternatively, the labeling agent may be a third moiety, such a secondary antibody that specifically binds to the antibody/OR complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see, e.g., Kronval et al., J. Immunol., 111:1401-1406 (1973); Akerstrom et al., J. Immunol., 135:2589-2542 (1985)). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

a. Non-competitive Assay Formats

Immunoassays for detecting an OR protein in a sample may be either competitive or noncompetitive. Noncompetitive immunoassays are assays in which the amount of antigen is directly measured. In one preferred “sandwich” assay, for example, the anti-OR antibodies can be bound directly to a solid substrate on which they are immobilized. These immobilized antibodies then capture the OR protein present in the test sample. The OR protein is thus immobilized is then bound by a labeling agent, such as a second OR antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

b. Competitive Assay Formats

In competitive assays, the amount of OR protein present in the sample is measured indirectly by measuring the amount of a known, added (exogenous) OR protein displaced (competed away) from an anti-OR antibody by the unknown OR protein present in a sample. In one competitive assay, a known amount of OR protein is added to a sample and the sample is then contacted with an antibody that specifically binds to the OR. The amount of exogenous OR protein bound to the antibody is inversely proportional to the concentration of OR protein present in the sample. In a particularly preferred embodiment, the antibody is immobilized on a solid substrate. The amount of OR protein bound to the antibody may be determined either by measuring the amount of OR protein present in a OR/antibody complex, or alternatively by measuring the amount of remaining uncomplexed protein. The amount of OR protein may be detected by providing a labeled OR molecule.

A hapten inhibition assay is another preferred competitive assay. In this assay the known OR protein is immobilized on a solid substrate. A known amount of anti-OR antibody is added to the sample, and the sample is then contacted with the immobilized OR. The amount of anti-OR antibody bound to the known immobilized OR protein is inversely proportional to the amount of OR protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

C. Cross-reactivity Determinations

Immunoassays in the competitive binding format can also be used for cross-reactivity determinations. For example, a protein at least partially encoded by the nucleic acid sequences disclosed herein can be immobilized to a solid support. Proteins (e.g., OR proteins and homologs) are added to the assay that compete for binding of the antisera to the immobilized antigen. The ability of the added proteins to compete for binding of the antisera to the immobilized protein is compared to the ability of the OR polypeptide encoded by the nucleic acid sequences disclosed herein to compete with itself. The percent cross-reactivity for the above proteins is calculated, using standard calculations. Those antisera with less than 10% cross-reactivity with each of the added proteins listed above are selected and pooled. The cross-reacting antibodies are optionally removed from the pooled antisera by immunoabsorption with the added considered proteins, e.g., distantly related homologs. In addition, peptides comprising amino acid sequences representing conserved motifs that are used to identify members of the OR family can be used in cross-reactivity determinations.

The immunoabsorbed and pooled antisera are then used in a competitive binding immunoassay as described above to compare a second protein, thought to be perhaps an allele or polymorphic variant of a OR family member, to the immunogen protein (i.e., OR protein encoded by the nucleic acid sequences disclosed herein). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by nucleic acid sequences disclosed herein required to inhibit 50% of binding, then the second protein is said to specifically bind to the polyclonal antibodies generated to a OR immunogen.

Antibodies raised against OR conserved motifs can also be used to prepare antibodies that specifically bind only to GPCRs of the OR family, but not to GPCRs from other families.

Polyclonal antibodies that specifically bind to a particular member of the OR family, e.g., AOLFR1, can be make by subtracting out cross-reactive antibodies using other OR family members. Species-specific polyclonal antibodies can be made in a similar way. For example, antibodies specific to human AOLFR1 can be made by, subtracting out antibodies that are cross-reactive with orthologous sequences, e.g., rat OR1 or mouse OR1.

d. Other Assay Formats

Western blot (immunoblot) analysis is used to detect and quantify the presence of OR protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the OR protein. The anti-OR polypeptide antibodies specifically bind to the OR polypeptide on the solid support. These antibodies may be directly labeled or alternatively may be subsequently detected using labeled antibodies (e.g., labeled sheep anti-mouse antibodies) that specifically bind to the anti-OR antibodies.

Other, assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev., 5:34-41 (1986)).

e. Reduction of non-specific Binding

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

f. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™) (SEQ ID NO: 529), fluorescent dyes (e.g., fluorescein isothiocyanate, Texas red, rhodamine, and the like), radiolabels (e.g., ³H, ¹²⁵I, ³⁵s, ¹⁴C, or ³²P), enzymes (e.g., horseradish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize a OR protein, or secondary antibodies that recognize anti-OR.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. I n this case, antigen-coated particles are agglutinated by samples comprising the target antibodies. In this format, none of the components need be labeled and the presence of the target antibody is detected by simple visual inspection.

E. Detection of Olfactory Modulators

Methods and compositions for determining whether a test compound specifically binds to a mammalian chemosensory, and more particularly, an olfactory receptor of the invention, both in vitro and in vivo are described below. Many aspects of cell physiology can be monitored to assess the effect of ligand-binding to a naturally-occurring or chimeric olfactory receptor. These assays may be performed on intact cells expressing an olfactory receptor, on permeabilized cells or on membrane fractions produced by standard methods.

Olfactory receptors are normally located on the specialized cilia of olfactory neurons. These receptors bind odorants and initiate the transduction of chemical stimuli into electrical signals. An activated or inhibited G protein will in turn alter the properties of target enzymes, channels, and other effector proteins. Some examples include the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

The OR protein of the assay will typically be selected from a polypeptide having a sequence selected from SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ID. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 111, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO., 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511, or conservatively modified variant thereof.

Alternatively, the OR protein of the assay can be derived from a eukaryote host cell and can include an amino acid subsequence having at least about 30-40% amino acid sequence identity to SEQ. ID. NO. 1, SEQ. ID. NO. 3, SEQ. ID. NO. 5, SEQ. ID. NO. 7, SEQ. ID. NO. 9, SEQ. ID. NO. 11, SEQ. ID. NO. 13, SEQ. ID. NO. 15, SEQ. ID. NO. 17, SEQ. ID. NO. 19, SEQ. ID. NO. 21, SEQ. ID. NO. 23, SEQ. ID. NO. 25, SEQ. ID. NO. 27, SEQ. ID. NO. 29, SEQ. ID. NO. 31, SEQ. ID. NO. 33, SEQ. ID. NO. 35, SEQ. ID. NO. 37, SEQ. ID. NO. 39, SEQ. ID. NO. 41, SEQ. ID. NO. 43, SEQ. ID. NO. 45, SEQ. ID. NO. 47, SEQ. ID. NO. 49, SEQ. ID. NO. 51, SEQ. ID. NO. 53, SEQ. ID. NO. 55, SEQ. ID. NO. 57, SEQ. ID. NO. 59, SEQ. ID. NO. 61, SEQ. ID. NO. 63, SEQ. ID. NO. 65, SEQ. ID. NO. 67, SEQ. ID. NO. 69, SEQ. ID. NO. 71, SEQ. ID. NO. 73, SEQ. ID. NO. 75, SEQ. ID. NO. 77, SEQ. ID. NO. 79, SEQ. ID. NO. 81, SEQ. ID. NO. 83, SEQ. ID. NO. 85, SEQ. ID. NO. 87, SEQ. ID. NO. 89, SEQ. ID. NO. 91, SEQ. ID. NO. 93, SEQ. ID. NO. 95, SEQ. ID. NO. 97, SEQ. ID. NO. 99, SEQ. ID. NO. 101, SEQ. ID. NO. 103, SEQ. ID. NO. 105, SEQ. ID. NO. 107, SEQ. ID. NO. 109, SEQ. ID. NO. 111, SEQ. ID. NO. 113, SEQ. ID. NO. 115, SEQ. ID. NO. 117, SEQ. ID. NO. 119, SEQ. ID. NO. 121, SEQ. ID. NO. 123, SEQ. ID. NO. 125, SEQ. ID. NO. 127, SEQ. ID. NO. 129, SEQ. ID. NO. 131, SEQ. ID. NO. 133, SEQ. ID. NO. 135, SEQ. ID. NO. 137, SEQ. ID. NO. 139, SEQ. ID. NO. 141, SEQ. ID. NO. 143, SEQ. ID. NO. 145, SEQ. ID. NO. 147, SEQ. ID. NO. 149, SEQ. ID. NO. 151, SEQ. ID. NO. 153, SEQ. ID. NO. 155, SEQ. ID. NO. 157, SEQ. ID. NO. 159, SEQ. ID. NO. 161, SEQ. ID. NO. 163, SEQ. ID. NO. 165, SEQ. ID. NO. 167, SEQ. ID. NO. 169, SEQ. ID. NO. 171, SEQ. ID. NO. 173, SEQ. ID. NO. 175, SEQ. ID. NO. 177, SEQ. ID. NO. 179, SEQ. ID. NO. 181, SEQ. ID. NO. 183, SEQ. ID. NO. 185, SEQ. ID. NO. 187, SEQ. ID. NO. 189, SEQ. ID. NO. 191, SEQ. ID. NO. 193, SEQ. ID. NO. 195, SEQ. ID. NO. 197, SEQ. ID. NO. 199, SEQ. ID. NO. 201, SEQ. ID. NO. 203, SEQ. ID. NO. 205, SEQ. ID. NO. 207, SEQ. ID. NO. 209, SEQ. ID. NO. 211, SEQ. ID. NO. 213, SEQ. ID. NO. 215, SEQ. ID. NO. 217, SEQ. ID. NO. 219, SEQ. ID. NO. 221, SEQ. ID. NO. 223, SEQ. ID. NO. 225, SEQ. ID. NO. 227, SEQ. ID. NO. 229, SEQ. ID. NO. 231, SEQ. ID. NO. 233, SEQ. ID. NO. 235, SEQ. ID. NO. 237, SEQ. ID. NO. 239, SEQ. ID. NO. 241, SEQ. ID. NO. 243, SEQ. ID. NO. 245, SEQ. ID. NO. 247, SEQ. ID. NO. 249, SEQ. ID. NO. 251, SEQ. ID. NO. 253, SEQ. ID. NO. 255, SEQ. ID. NO. 257, SEQ. ID. NO. 259, SEQ. ID. NO. 261, SEQ. ID. NO., 263, SEQ. ID. NO., 265, SEQ. ID. NO. 267, SEQ. ID. NO. 269, SEQ. ID. NO. 271, SEQ. ID. NO. 273, SEQ. ID. NO. 275, SEQ. ID. NO. 277, SEQ. ID. NO. 279, SEQ. ID. NO. 281, SEQ. ID. NO. 283, SEQ. ID. NO. 285, SEQ. ID. NO. 287, SEQ. ID. NO. 289, SEQ. ID. NO. 291, SEQ. ID. NO. 293, SEQ. ID. NO. 295, SEQ. ID. NO. 297, SEQ. ID. NO. 299, SEQ. ID. NO. 301, SEQ. ID. NO. 303, SEQ. ID. NO. 305, SEQ. ID. NO. 307, SEQ. ID. NO. 309, SEQ. ID. NO. 311, SEQ. ID. NO. 313, SEQ. ID. NO. 315, SEQ. ID. NO. 317, SEQ. ID. NO. 319, SEQ. ID. NO. 321, SEQ. ID. NO. 323, SEQ. ID. NO. 325, SEQ. ID. NO. 327, SEQ. ID. NO. 329, SEQ. ID. NO. 331, SEQ. ID. NO. 333, SEQ. ID. NO. 335, SEQ. ID. NO. 337, SEQ. ID. NO. 339, SEQ. ID. NO. 341, SEQ. ID. NO. 343, SEQ. ID. NO. 345, SEQ. ID. NO. 347, SEQ. ID. NO. 349, SEQ. ID. NO. 351, SEQ. ID. NO. 353, SEQ. ID. NO. 355, SEQ. ID. NO. 357, SEQ. ID. NO. 359, SEQ. ID. NO. 361, SEQ. ID. NO. 363, SEQ. ID. NO. 365, SEQ. ID. NO. 367, SEQ. ID. NO. 369, SEQ ID NO: 371, SEQ. ID. NO. 373, SEQ. ID. NO. 375, SEQ. ID. NO. 377, SEQ. ID. NO. 379, SEQ. ID. NO. 381, SEQ. ID. NO. 383, SEQ. ID. NO. 385, SEQ. ID. NO. 387, SEQ. ID. NO. 389, SEQ. ID. NO. 391, SEQ. ID. NO. 393, SEQ. ID. NO. 395, SEQ. ID. NO. 397, SEQ. ID. NO. 399, SEQ. ID. NO. 401, SEQ. ID. NO. 403, SEQ. ID. NO. 405, SEQ. ID. NO. 407, SEQ. ID. NO. 409, SEQ. ID. NO. 411, SEQ. ID. NO. 413, SEQ. ID. NO. 415, SEQ. ID. NO. 417, SEQ. ID. NO. 419, SEQ. ID. NO. 421, SEQ. ID. NO. 423, SEQ. ID. NO. 425, SEQ. ID. NO. 427, SEQ. ID. NO. 429, SEQ. ID. NO. 431, SEQ. ID. NO. 433, SEQ. ID. NO. 435, SEQ. ID. NO. 437, SEQ. ID. NO. 439, SEQ. ID. NO. 441, SEQ. ID. NO. 443, SEQ. ID. NO. 445, SEQ. ID. NO. 447, SEQ. ID. NO. 449, SEQ. ID. NO. 451, SEQ. ID. NO. 453, SEQ. ID. NO. 455, SEQ. ID. NO. 457, SEQ. ID. NO. 459, SEQ. ID. NO. 461, SEQ. ID. NO. 463, SEQ. ID. NO. 465, SEQ. ID. NO. 467, SEQ. ID. NO. 469, SEQ. ID. NO. 471, SEQ. ID. NO. 473, SEQ. ID. NO. 475, SEQ. ID. NO. 477, SEQ. ID. NO. 479, SEQ. ID. NO. 481, SEQ. ID. NO. 483, SEQ. ID. NO. 485, SEQ. ID. NO. 487, SEQ. ID. NO. 489, SEQ. ID. NO. 491, SEQ. ID. NO. 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

Preferably, the amino acid sequence identity will be at least 50-75% preferably 85%, 90%, 95%, 96%, 97%, 98%, or 99%. Optionally, the polypeptide of the assays can comprise a domain of an OR protein, such as an extracellular domain, transmembrane region, transmembrane domain, cytoplasmic domain, ligand-binding domain, subunit association domain, active site, and the like. Either the OR protein or a domain thereof can be covalently linked to a heterologous protein to create a chimeric protein used in the assays described herein. As discussed infra, the family of ORs provided herein exhibits substantial sequence similarity at both the DNA and protein level, but also significant dissimilarly. In particular, the members possess an average percentage sequence identity to other members of the family when determined over the full length of the gene by about 30%. Moreover, different members of the genes at the protein level exhibit an average on the order of about 40% sequence identity to other members of the family when the full length protein sequences are compared. However, while there exist differences, there are characteristic similarities, e.g. the consensus sequence already mentioned, which further define members of this novel genus of receptors.

Modulators of OR activity can be tested using OR polypeptides as described above, either recombinant or naturally occurring. The protein can be isolated, expressed in a cell, expressed in a membrane derived from a cell, expressed in tissue or in an animal, either recombinant or naturally occurring. Modulation can be tested using one of the in vitro or in vivo assays described herein.

1. In vitro Binding Assays

Olfactory transduction can also be examined in vitro with soluble or solid state reactions, using a full-length OR or a chimeric molecule such as an extracellular domain or transmembrane region, or combination thereof, of a OR covalently linked to a heterologous signal transduction domain, or a heterologous extracellular domain and/or transmembrane region, covalently linked to the transmembrane and/or cytoplasmic domain of an OR. Furthermore, ligand-binding domains of the protein of interest can be used in vitro in soluble or solid state reactions to assay for ligand binding. In numerous embodiments, a chimeric receptor will be made that comprises all or part of a OR polypeptide, as well an additional sequence that facilitates the localization of the OR to the membrane, such as a rhodopsin, e.g., an N-terminal fragment of a rhodopsin protein, e.g. bovine or another mammalian rhodopsin.

Ligand binding to a OR protein, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbence, refractive index) hydrodynamic (e.g., shape), chromatographic, or solubility properties.

Receptor-G protein interactions can also be examined. For example, binding of the G protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors, e.g., by adding an activator to the receptor and G protein in the absence of GTP, which form a tight complex, and then screen for inhibitors by looking at dissociation of the receptor-G protein complex. In the presence of GTP, release of the alpha subunit of the G protein from the other two G protein subunits serves as a criterion of activation.

An activated or inhibited G protein will in turn alter the properties of target enzymes, channels, and other effector proteins. The classic examples are the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

In another embodiment of the invention, a GTPγS assay may be used. As described above, upon activation of a GPCR, the Gα subunit of the G protein complex is stimulated to exchange bound GDP for GTP. Ligand-mediated stimulation of G protein exchange activity can be measured in a biochemical assay measuring the binding of added radioactively-labeled GTPγ³⁵S to the G protein in the presence of a putative ligand. Typically, membranes containing the chemosensory receptor of interest are mixed with a complex of G proteins. Potential inhibitors and/or activators and GTPγS are added to the assay, and binding of GTPγS to the G protein is measured. Binding can be measured by liquid scintillation counting or by any other means known in the art, including scintillation proximity assays (SPA). In other assays formats, fluorescently-labeled GTPγS can be utilized.

2. Fluorescence Polarization Assays

In another embodiment, Fluorescence Polarization (“FP”) based assays may be used to detect and monitor odorant binding. Fluorescence polarization is a versatile laboratory technique for measuring equilibrium binding, nucleic acid hybridization, and enzymatic activity. Fluorescence polarization assays are homogeneous in that they do not require a separation step such as centrifugation, filtration, chromatography, precipitation or electrophoresis. These assays are done in real time, directly in solution and do not require an immobilized phase. Polarization values can be measured repeatedly and after the addition of reagents since measuring the polarization is rapid and does not destroy the sample. Generally, this technique can be used to measure polarization values of fluorophores from low picomolar to micromolar levels. This section describes how fluorescence polarization can be used in a simple and quantitative way to measure the binding of odorants to the olfactory receptors of the invention.

When a fluorescently labeled molecule is excited with plane polarized light, it emits light that has a degree of polarization that is inversely proportional to its molecular rotation. Large fluorescently labeled molecules remain relatively stationary during the excited state (4 nanoseconds in the case of fluorescein) and the polarization of the light remains relatively constant between excitation and emission. Small fluorescently labeled molecules rotate rapidly during the excited state and the polarization changes significantly between excitation and emission. Therefore, small molecules have low polarization values and large molecules have high polarization values. For example, a single-stranded fluorescein-labeled oligonucleotide has a relatively low polarization value but when it is hybridized to a complementary strand, it has a higher polarization value. When using FP to detect and monitor odorant-binding which may activate or inhibit the olfactory receptors of the invention, fluorescence-labeled odorants or auto-fluorescent odorants may be used.

Fluorescence polarization (P) is defined as:

$$P = \frac{{Int}_{∐} - {Int}_{⊥}}{{Int}_{∐} + {Int}_{⊥}}$$

Where [symbol] is the intensity of the emission light parallel to the excitation light plane and Int [symbol] is the intensity of the emission light perpendicular to the excitation light plane. P, being a ratio of light intensities, is a dimensionless number. For example, the Beacon ® and Beacon 2000 ™ System may be used in connection with these assays. Such systems typically express polarization in millipolarization units (1 Polarization Unit=1000 mP Units).

The relationship between molecular rotation and size is described by the Perrin equation and the reader is referred to Jolley, M. E. (1991) in Journal of Analytical Toxicology, pp. 236-240, which gives a thorough explanation of this equation. Summarily, the Perrin equation states that polarization is directly proportional to the rotational relaxation time, the time that it takes a molecule to rotate through an angle of approximately 68.5° Rotational relaxation time is related to viscosity (η), absolute temperature (T), molecular volume (V), and the gas constant (R) by the following equation:

$$Rotational Relaxation Time = \frac{3 η V}{RT}$$

The rotational relaxation time is small (≈1 nanosecond) for small molecules (e.g. fluorescein) and large (≈100 nanoseconds) for large molecules (e.g. immunoglobulins). If viscosity and temperature are held constant, rotational relaxation time, and therefore polarization, is directly related to the molecular volume. Changes in molecular volume may be due to interactions with other molecules, dissociation, polymerization, degradation, hybridization, or conformational changes of the fluorescently labeled molecule. For example, fluorescence polarization has been used to measure enzymatic cleavage of large fluorescein labeled polymers by proteases, DNases, and RNases. It also has been used to measure equilibrium binding for protein/protein interactions, antibody/antigen binding, and protein/DNA binding.

3. Solid state and Soluble High Throughput Assays

In yet another embodiment, the invention provides soluble assays using molecules such as a domain such as ligand-binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; an OR protein; or a cell or tissue expressing an OR protein, either naturally occurring or recombinant. In another embodiment, the invention provides solid phase based in vitro assays in a high throughput format, where the domain, chimeric molecule, OR protein, or cell or tissue expressing the OR is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 1000 to about 1500 different compounds. It is also possible to assay multiple compounds in each plate well. Further, it is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non covalent linkage, e.g., via a tag. The tag can be any of a variety of components. In general, a molecule which binds the tag (a tag binder) is fixed to a solid support, and the tagged molecule of interest (e.g., the olfactory transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface which is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc., 85:2149-54 (1963) (describing solid phase synthesis of, e.g., peptides); Geysen et al., J. Immun. Meth., 102:259-74 (1987) (describing synthesis of solid phase components on pins); Frank & Doring, Tetrahedron, 44:60316040 (1988) (describing synthesis of various peptide sequences on cellulose disks); Fodor et al., Science, 251:767-77 (1991); Sheldon et al., Clinical Chemistry, 39(4):718-19 (1993); and Kozal et al., Nature Medicine, 2(7):753759 (1996) (all describing arrays of biopolymers fixed to solid substrates). Non-chemical approaches for fixing tag binders to substrates include other common methods, such as heat, cross-linking by UV radiation, and the like.

4. Computer-based Assays

Yet another assay for compounds that modulate OR protein activity involves computer assisted compound design, in which a computer system is used to generate a three-dimensional structure of an OR protein based on the structural information encoded by its amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a OR polypeptide into the computer system. The nucleotide sequence encoding the polypeptide, or the amino acid sequence thereof, can be any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ BD NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511, and conservatively modified versions thereof.

The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art. .

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand-binding regions are identified by the computer system. Three-dimensional structures for potential ligands are generated by entering amino acid or nucleotide sequences or chemical formulas of compounds, as described above. The three-dimensional structure of the potential ligand is then compared to that of the OR protein to identify ligands that bind to the protein. Binding affinity between the protein and ligands is determined using energy terms to determine which ligands have an enhanced probability of binding to the protein.

Computer systems are also used to screen for mutations, polymorphic variants, alleles and interspecies homologs of OR genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated OR genes involves receiving input of a first nucleic acid or amino acid sequence of a OR gene, or conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in various OR genes, and mutations associated with disease states and genetic traits.

5. Cell-based Binding Assays

In a preferred embodiment, an OR polypeptide is expressed in a eukaryotic cell as a chimeric receptor with a heterologous, chaperone sequence that facilitates its maturation and targeting through the secretory pathway. In a preferred embodiment, the heterologous sequence is a rhodopsin sequence, such as an N-terminal fragment of a rhodopsin. Such chimeric OR receptors can be expressed in any eukaryotic cell, such as HEK-293 cells. Preferably, the cells comprise a functional G protein, e.g., Gα15, that is capable of coupling the chimeric receptor to an intracellular signaling pathway or to a signaling protein such as phospholipase C. Activation of such chimeric receptors in such cells can be detected using any standard method, such as by detecting changes in intracellular calcium by detecting FURA-2 dependent fluorescence in the cell.

Activated GPCR receptors become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G proteins. The kinase/arrestin pathway plays a key role in the desensitization of many GPCR receptors. For example, compounds that modulate the duration an olfactory receptor stays active would be useful as a means of prolonging a desired odor or cutting off an unpleasant one. For a general review of GPCR signal transduction and methods of assaying signal transduction, see, e.g., Methods in Enzymology, vols. 237 and 238 (1994) and volume 96 (1983); Bourne et al., Nature, 10:349:117-27 (1991); Bourne et al., Nature, 348:125-32 (1990); Pitcher et al., Annu. Rev. Biochem., 67:653-92 (1998).

OR modulation may be assayed by comparing the response of an OR polypeptide treated with a putative OR modulator to the response of an untreated control sample. Such putative OR modulators can include odorants that either inhibit or activate OR polypeptide activity. In one embodiment, control samples (untreated with activators or inhibitors) are assigned a relative OR activity value of 100. Inhibition of an OR polypeptide is achieved when the OR activity value relative to the control is about 90%, optionally 50%, optionally 25-0%. Activation of an OR polypeptide is achieved when the OR activity value relative to the control is 110%, optionally 150%, 200-500%, or 1000-2000%.

Changes in ion flux may be assessed by determining changes in polarization (i.e., electrical potential) of the cell or membrane expressing a OR protein. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J Med., 336:1575-1595 (1997)). Whole cell currents are conveniently determined using the standard. Other known assays include: radiolabeled ion flux assays and fluorescence assays using voltage-sensitive dyes (see, e.g., Vestergarrd-Bogind et al., J. Membrane Biol., 88:67-75 (1988); Gonzales & Tsien, Chem. Biol., 4:269277 (1997); Daniel et al., J. Pharmacol. Meth., 25:185-193 (1991); Holevinsky et al., J. Membrane Biology, 137:59-70 (1994)). Generally, the compounds to be tested are present in the range from 1 pM to 100 mM.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP3, cGMP, or cAMP.

Preferred assays for GPCRs include cells that are loaded with ion or voltage sensitive dyes to report receptor activity. Assays for determining activity of such receptors can also use known agonists and antagonists for other G protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G protein coupled receptors, promiscuous G proteins such as Gα15 and Gα16 can be used in the assay of choice (Wilkie et al., PNAS, 88:10049-53 (1991)). Such promiscuous G proteins allow coupling of a wide range of receptors.

Receptor activation typically initiates subsequent intracellular events, e.g., increases in second messengers such as IP3, which releases intracellular stores of calcium ions. Activation of some G protein coupled receptors stimulates the formation of inositol triphosphate (IP3) through phospholipase C-mediated hydrolysis of phosphatidylinositol (Berridge & Irvine, Nature, 312:315-21 (1984)). IP3 in turn stimulates the release of intracellular calcium ion stores. Thus, a change in cytoplasmic calcium ion levels, or a change in second messenger levels such as IP3 can be used to assess G protein coupled receptor function. Cells expressing such G protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated, result in a change in the level of intracellular cyclic nucleotides, e.g., cAMP or cGMP, by activating or inhibiting enzymes such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., PNAS, 88:9868-72 (1991) and Dhallan et al., Nature, 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-crated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In a preferred embodiment, OR protein activity is measured by expressing a OR gene in a heterologous cell with a promiscuous G protein that links the receptor to a phospholipase C signal transduction pathway (see Offermanns & Simon, J. Biol. Chem., 270:15175-15180 (1995)). Optionally the cell line is HEK-293 (which does not naturally express OR genes) and the promiscuous G protein is Gα15Gα16 (Offermanns & Simon, supra). Modulation of olfactory transduction is assayed by measuring changes in intracellular Ca²⁺ levels, which change in response to modulation of the OR signal transduction pathway via administration of a molecule that associates with a OR protein. Changes in Ca²⁺ levels are optionally measured using fluorescent Ca²⁺ indicator dyes and fluorometric imaging.

In one embodiment, the changes in intracellular cAMP or cGMP can be measured using immunoassays. The method described in Offermanns & Simon, J. Bio. Chem., 270:15175-15180 (1995), may be used to determine the level of cAMP. Also, the method described in Felley-Bosco et al., Am. J. Resp. Cell and Mol. Biol., 11:159-164 (1994), may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with 3H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist, to cpm in the presence of buffer control. Likewise, fold inhibition is determined by calculating the ratio of cpm in the presence of antagonist, to cpm in the presence of buffer control (which may or may not contain an agonist).

In another embodiment, transcription levels can be measured to assess the effects of a test compound on signal transduction. A host cell containing an OR protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, luciferase, '3-galactosidase and alkaline phosphatase. Furthermore, the protein of interest can be used as an indirect reporter via attachment to a second reporter such as green fluorescent protein (see, e.g., Mistili & Spector, Nature Biotechnology, 15:961-64 (1997)).

The amount of transcription is then compared to the amount of transcription in either the same cell in the absence of the test compound, or it may be compared with the amount of transcription in a substantially identical cell that lacks the OR protein of interest. A substantially identical cell may be derived from the same cells from which the recombinant cell was prepared but which had not been modified by introduction of heterologous DNA. Any difference in the amount of transcription indicates that the test compound has in some manner altered the activity of the OR protein of interest.

6. Transgenic non-human Animals Expressing Olfactory Receptors

Non-human animals expressing one or more olfactory receptor sequences of the invention, particularly human olfactory receptor sequences, can also be used for receptor assays. Such expression can be used to determine whether a test compound specifically binds to a mammalian olfactory transmembrane receptor polypeptide in vivo by contacting a non-human animal stably or transiently transfected with a nucleic acid encoding an olfactory receptor or ligand-binding region thereof with a test compound and determining whether the animal reacts to the test compound by specifically binding to the receptor polypeptide.

Use of the translocation domains of the invention in the fusion polypeptides generates a cell expressing high levels of olfactory receptor. Animals transfected or infected with the vectors of the invention are particularly useful for assays to identify and characterize odorants/ligands that can bind to a specific or sets of receptors. Such vector-infected animals expressing libraries of human olfactory sequences can be used for in vivo screening of odorants and their effect on, e.g., cell physiology (e.g., on olfactory neurons), on the CNS (e.g., olfactory bulb activity), or behavior.

Means to infect/express the nucleic acids and vectors, either individually or as libraries, are well known in the art. A variety of individual cell, organ or whole animal parameters can be measured by a variety of means. For example, recording of stimulant-induced waves (bulbar responses) from the main olfactory bulb or accessory olfactory bulb is a useful tool for measuring quantitative stable olfactory responses. When electrodes are located on the olfactory bulb surface it is possible to record stable responses over a period of several days (see, e.g., Kashiwayanagi, Brain Res. Protoc. 1:287-291 (1997)). In this study, electroolfactogram recordings were made with a four-electrode assembly from the olfactory epithelium overlying the endoturbinate bones facing the nasal septum. Four electrodes were fixed along the dorsal-to-ventral axis of one turbinate bone or were placed in corresponding positions on four turbinate bones and moved together up toward the top of the bone. See also, Scott, J. Neurophysiol. 77:1950-1962 (1997); Scott, J. Neurophysiol. 75:2036-2049 (1996); Ezeh, J. Neurophysiol. 73:2207-2220 (1995). In other systems, fluorescence changes in nasal epithelium can be measured using the dye di-4-ANEPPS, which is applied on the rat's nasal septum and medial surface of the turbinates (see, e.g., Youngentob, J. Neurophysiol. 73:387-398 (1995)). Extracellular potassium activity (aK) measurements can also be carried out in in vivo. An increase in aK can be measured in the mucus and the proximal part of the nasal epithelium (see, e.g., Khayari, Brain Res. 539:1-5 (1991)).

The OR sequences of the invention can be for example expressed in animal nasal epithelium by delivery with an infecting agent, e.g., adenovirus expression vector. Recombinant adenovirus-mediated expression of a recombinant gene in olfactory epithelium using green fluorescent protein as a marker is described by, e.g., Touhara, PNAS, 96:4040-45 (1999).

The endogenous olfactory receptor genes can remain functional and wild-type (native) activity can still be present. In other situations, where it is desirable that all olfactory receptor activity is by the introduced exogenous hybrid receptor, use of a knockout line is preferred. Methods for the construction of non-human transgenic animals, particularly transgenic mice, and the selection and preparation of recombinant constructs for generating transformed cells are well known in the art.

Construction of a “knockout” cell and animal is based on the premise that the level of expression of a particular gene in a mammalian cell can be decreased or completely abrogated by introducing into the genome a new DNA sequence that serves to interrupt some portion of the DNA sequence of the gene to be suppressed. Also, “gene trap insertion” can be used to disrupt a host gene, and mouse embryonic stem (ES) cells can be used to produce knockout transgenic animals (see, e.g., Holzschu, Transgenic Res 6:97-106 (1997)). The insertion of the exogenous is typically by homologous recombination between complementary nucleic acid sequences. The exogenous sequence is some portion of the target gene to be modified, such as exonic, intronic or transcriptional regulatory sequences, or any genomic sequence which is able to affect the level of the target gene's expression; or a combination thereof. Gene targeting via homologous recombination in pluripotential embryonic stem cells allows one to modify precisely the genomic sequence of interest. Any technique can be used to create, screen for, propagate, a knockout animal, e.g., see Bijvoet, Hum. Mol. Genet. 7:53-62 (1998); Moreadith, J. Mol. Med. 75:208-216 (1997); Tojo, Cytotechnology 19:161-165 (1995); Mudgett, Methods Mol. Biol. 48:167-184 (1995); Longo, Transgenic Res. 6:321-328 (1997); U.S. Pat. Nos. 5,616,491; 5,464,764; 5,631,153; 5,487,992; 5,627,059; 5,272,071; WO 91/09955; WO 93/09222; WO 96/29411; WO 95/31560; WO 91/12650.

The nucleic acid libraries of the invention can also be used as reagents to produce “knockout” human cells and their progeny. Likewise, the nucleic acids of the invention can also be used as reagents to produce “knock-ins” in mice. The human or rat OR gene sequences can replace the orthologous ORs in the mouse genome. In this way, a mouse expressing a human or rat OR can be produced. This mouse can then be used to analyze the function of human or rat ORs, and to identify ligands for such ORs.

F. Modulators

The compounds tested as modulators of an OR family member can be any small chemical compound, or a biological entity, such as a protein, sugar, nucleic acid or lipid. Alternatively, modulators can be genetically altered versions of an OR gene. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

The OR modulating compounds can be used in any number of consumer products, including, but not limited to, purfumes, fragrance compositions, deorderants, air fresheners, foods, drugs, etc., or ingredients thereof, to thereby modulate the odor of the product, composition, or ingredient in a desired manner. As one of skill in the art will recognize, OR modulating compounds can be used to enhance desireable odors, to block malodors, or a combination thereof.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual odorant compositions.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-93 (1991) and Houghton et al., Nature, 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., PNAS, 90:6909-13 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-18 (1992)), analogous organic syntheses of small compound libraries (Chen et al., J. Amer. Chem. Soc., 116:2661 (1994)), oligocarbamates (Cho et al., Science, 261:1303 (1993)), peptidyl phosphonates (Campbell et al., J. Org. Chem., 59:658 (1994)), nucleic acid libraries (Ausubel, Berger and Sambrook, all supra), peptide nucleic acid libraries (U.S. Pat. No. 5,539,083), antibody libraries (Vaughn et al., Nature Biotechnology, 14(3):309-14 (1996) and PCT/US96/10287), carbohydrate libraries (Liang et al., Science, 274:1520-22 (1996) and U.S. Pat. No. 5,593,853), small organic molecule libraries (benzodiazepines, Baum, C&EN, Jan 18, page 33 (1993); thiazolidinones and metathiazanones, U.S. Pat. No. 5,549,974; pynrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., 357 MPS, 390 MPS (Advanced Chem Tech, Louisville Ky.), Symphony (Rainin, Woburn, Mass.), 433A (Applied Biosystems, Foster City, Calif.), 9050 Plus (Millipore, Bedford, Mass.)). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences; Columbia, Md.; etc.).

G. Methods for Representing and Predicting the Perception of Odor

The invention also preferably provides methods for representing the perception of odor (or taste) and/or for predicting the perception of odor (or taste) in a mammal, including in a human. Preferably, such methods may be performed by using the receptors and genes encoding said olfactory receptors disclosed herein.

Also contemplated as within the invention, is a method of screening one or more compounds for the presence of an odor detectable by a mammal, comprising: contacting said one or more compounds with the disclosed receptors, preferably wherein the mammal is a human. Also contemplated as within the invention is a method for representing olfactory perception of a particular smell in a mammal, comprising: providing values X₁ to Xₙ representative of the quantitative stimulation of each of n olfactory receptors of said vertebrate, where n is greater than or equal to 4; and generating from said values a quantitative representation of olfactory perception. The olfactory receptors may be an olfactory receptor disclosed herein, the representation may constitutes a point or a volume in n-dimensional space, may constitutes a graph or a spectrum, and may constitutes a matrix of quantitative representations. Also, the providing step may comprise contacting a plurality of recombinantly-produced olfactory receptors with a test composition and quantitatively measuring the interaction of said composition with said receptors.

Also contemplated as within the invention, is a method for predicting the olfactory perception in a mammal generated by one or more molecules or combinations of molecules yielding unknown olfactory perception in a mammal, comprising: providing values X₁ to Xₙ representative of the quantitative stimulation of each of n olfactory receptors of said vertebrate, where n is greater than or equal to 4, for one or more molecules or combinations of molecules yielding known olfactory perception in a mammal; and generating from said values a quantitative representation of olfactory perception in a mammal for the one or more molecules or combinations of molecules yielding known olfactory perception in a mammal, providing values X₁ to Xₙ representative of the quantitative stimulation of each of n olfactory receptors of said vertebrate, where n is greater than or equal to 4, for one or more molecules or combinations of molecules yielding unknown olfactory perception in a mammal; and generating from said values a quantitative representation of olfactory perception in a mammal for the one or more molecules or combinations of molecules yielding unknown olfactory perception in a mammal, and predicting the olfactory perception in a mammal generated by one or more molecules or combinations of molecules yielding unknown olfactory perception in a mammal by comparing the quantitative representation of olfactory perception in a mammal for the one or more molecules or combinations of molecules yielding unknown olfactory perception in a mammal to the quantitative representation of olfactory perception in a mammal for the one or more molecules or combinations of molecules yielding known olfactory perception in a mammal. The olfactory receptors used in this method may include an olfactory receptor disclosed herein.

In another embodiment, novel molecules or combinations of molecules are generated which elicit a predetermined olfactory perception in a mammal by determining a value of olfactory perception in a mammal for a known molecule or combinations of molecules as described above; determining a value of olfactory perception in a mammal for one or more unknown molecules or combinations of molecules as described above; comparing the value of olfactory perception in a mammal for one or more unknown compositions to the value of olfactory perception in a mammal for one or more known compositions; selecting a molecule or combination of molecules that elicits a predetermined olfactory perception in a mammal; and combining two or more unknown molecules or combinations of molecules to form a molecule or combination of molecules that elicits a predetermined olfactory perception in a mammal. The combining step yields a single molecule or a combination of molecules that elicits a predetermined olfactory perception in a mammal.

In another embodiment of the invention, there is provided a method for simulating a fragrance, comprising: for each of a plurality of cloned olfactory receptors, preferably human receptors, ascertaining the extent to which the receptor interacts with the fragrance; and combining a plurality of compounds, each having a previously-ascertained interaction with one or more of the receptors, in amounts that together provide a receptor-stimulation profile that mimics the profile for the fragrance. Interaction of a fragrance with an olfactory receptor can be determined using any of the binding or reporter assays described herein. The plurality of compounds may then be combined to form a mixture. If desired, one or more of the plurality of the compounds can be combined covalently. The combined compounds substantially stimulate at least 75%, 80% or 90% of the receptors that are substantially stimulated by the fragrance.

In another preferred embodiment of the invention, a plurality of standard compounds are tested against a plurality of olfactory receptors to ascertain the extent to which the receptors each interact with each standard compound, thereby generating a receptor stimulation profile for each standard compound. These receptor stimulation profiles may then be stored in a relational database on a data storage medium. The method may further comprise providing a desired receptor-stimulation profile for a scent; comparing the desired receptor stimulation profile to the relational database; and ascertaining one or more combinations of standard compounds that most closely match the desired receptor-stimulation profile. The method may further comprise combining standard compounds in one or more of the ascertained combinations to simulate the scent.

H. Kits

OR genes and their homologs are useful tools for identifying olfactory receptor cells, for forensics and paternity determinations, and for examining olfactory transduction. OR family member-specific reagents that specifically hybridize to OR nucleic acids, such as AOLFR1 probes and primers, and OR family member-specific reagents that specifically bind to an OR protein, e.g., OR antibodies are used to examine olfactory cell expression and olfactory transduction regulation.

Nucleic acid assays for the presence of DNA and RNA for an OR family member in a sample include numerous techniques are known to those skilled in the art, such as southern analysis, northern analysis, dot blots, RNase protection, S1 analysis, amplification techniques such as PCR, and in situ hybridization. In in situ hybridization, for example, the target nucleic acid is liberated from its cellular surroundings in such a form so as to be available for hybridization within the cell, while preserving the cellular morphology for subsequent interpretation and analysis. The following articles provide an overview of the art of in situ hybridization: Singer et al., Biotechniques, 4:230-50 (1986); Haase et al., Methods in Virology, vol. VII, pp. 189-226 (1984); and Nucleic Acid Hybridization: A Practical Approach (Names et al., eds. 1987). In addition, an OR protein can be detected with the various immunoassay techniques described above. The test sample is typically compared to both a positive control (e.g., a sample expressing a recombinant OR protein) and a negative control.

The present invention also provides for kits for screening for modulators of OR family members. Such kits can be prepared from readily available materials and reagents. For example, such kits can comprise any one or more of the following materials: OR nucleic acids or proteins, reaction tubes, and instructions for testing OR activity. Optionally, the kit contains a biologically active OR receptor. A wide variety of kits and components can be prepared according to the present invention, depending upon the intended user of the kit and the particular needs of the user.

### EXAMPLES

Genomic, predicted amino acid sequence, and predicted coding sequences (cds), of novel G protein-coupled human odorant receptors, and classes of such receptors, are described. Each example describes a discrete protein and nucleic acid pair. Accordingly, Example 1 describes SEQ. ID. NOS. 1 and 2, for the human olfactory receptor protein designated AOLFR1, and the human DNA encoding

AOLFR1, respectively; Example 2 describes SEQ. ID. NOS. 3 and 4, for the human olfactory receptor protein designated AOLFR2, and the human DNA encoding

AOLFR2, respectively; and so on in 'the manner described, through the final Example sequence.

In the protein sequences presented herein, the one-letter code X or Xaa refers to any of the twenty common amino acid residues. In the DNA sequences presented herein, the one letter codes N or n refers to any of the of the four common nucleotide bases, A, T, C, or G.

### EXAMPLES

| | |
|---|---|
| AOLFR1 sequences: | |
| MKTFSSFLQIGNMHQGNQTTITEFLLGFFKQDNLLFVLFLGMYLVTVIGNGLIIVAISLD | (SEQ ID NO:1) |
| TYLHTPMYLFLANLSFADISSISNSVPKMLVNIQTKSQSISYESCITQMYFSIVFVVIDNLLLGTM | |
| AYDHFVAICHPLNYTILMRPRFGILLTVISWFLSNIIALTHTLLLIQLLFCNHNTLPHFFCDLAPLL | |
| KLSCSDTLINELVLFIVGLSVIIFPFTLSSFFSYVCIIRAVLRVSSTQGKWKAFSTCGSHLTVVLLFY | |
| GTIVGVYFFPSSTHPEDTDKIGAVLFTVVTPMINPFIYSLRNKDMKGALRKLNRKISSL | |
| ATGAAGACTTTTAGTTCCTTTCTTCAGATCGGCAGAAATATGCATCAAGGAAACCAAACCA | (SEQ ID NO:2) |
| AACTCACTGAATTCATTCTCCTGGGATTTTTCAAGCAGGATGAGCATCAAAACCTCCTCTTT | |
| GTGCTTTTCTTGGGTATGTACCTGGTCACTGTGATTGGGAACGGGCTCATCATTGTGGCTA | |
| TCAGCTTGGATACGTACCTTCATACCCCCATGTATCTCTTCCTTGCCAATCTATCCTTTGCT | |
| GATATTTCCTCCATTTCCAACTCAGTCCCCAAAATGCTGGTGAATATTCAAACCAAGAGTC | |
| AATCCATCTCTTATGAGAGCTGCATCACACAGATGTACTTTTCTATTGTGTTTGTCGTCATT | |
| GACAATTTGCTCTTGGGGACCATGGCCTATGACGACTTTGTGGCGATCTGCCACCCTCTGA | |
| ATTATACAATTCTCATGCGGCCCAGGTTCGGCATTTTGCTGACAGTCATCTCATGGTTCCTC | |
| AGTAATATTATTGCTCTGACACACACCCTTCTGCTCATTCAATTGCTCTTCTGTAACCACAA | |
| CACTCTGCCACACTTCTTCTGTGACTTGGCCCCTCTGCTCAAACTGTCCTGTTCAGATACAT | |
| TGATCAATGAGCTTGTGTTGTTTATTGTGGGTTTATCAGTTATCATCTTCCCCTTTACACTC | |
| AGCTTCTTTTCCTATGTCTGCATCATCAGAGCTGTCCTGAGAGTATCTTCCACACAGGGAA | |
| AGTGGAAAGCGTTCTCCACTTGTGGCTCTCACCTGACAGTTGTATTACTGTTCTACGGAAC | |
| CATTGTAGGCGTGTACTTTTTCCCCTCCTCCACTCACCCTGAGGACACTGATAAGATTGGT | |
| GCTGTCCTATTCACTGTGGTGACACCCATGATAAACCCCTTCATCTACAGCTTGAGGAATA | |
| AGGATATGAAAGGTGCCCTGAGAAAGCTCATCAATAGAAAAATTTCTTCCCTTTGA | |
| AOLFR2 sequences: | |
| MMMVLRNLSMEPTFALLGFTDYPKLQIPLFLVFLLMYVITVVGNLGMIIIIKINPKFHTPMYFFL | (SEQ ID NO:3) |
| SHLSFVDFCYSSIVTPKLLENLVMADKSIFYFSCMMQYFLSCTAVVTESFLLAVMAYDRIFVAIC | |
| NPLLYTVAMSQRLCALLVAGSYLWGMFGPLVLLCYALRLNFSGPNVINHFFCEYTALISVSGS | |
| DILIPHLLLFSFATFNEMCTLLIILTSYVFIFVTVLKIRSVSGRHKAFSTWASHLTAITIFHGTILFL | |
| YCVPNSKNSRQTVKVASVFYTVVNPMLNPPIYSLRNKDVKDAFWKLIHTQVPFH | |
| ATGATGATGGTTTTAAGGAATCTGAGCATGGAGCCCACCTTTGCCCTTTTAGGTTTCACAG | (SEQ ID NO:4) |
| ATTACCCAAAGCTTCAGATTCCTCTCTTCGTTGTGTTTCTGCTCATGTATGTTATGACAGTG | |
| GTAGGAAACCTTGGGATGATCATAATAATCAAGATTAACCCCAAATTTCACACTCCTATGT | |
| ACTTTTTCCTTAGTCACCTCTCTTTTGTTGATTTTTGTTACTCTTCCATTGTCACTCCCAAGC | |
| TGCTTGAGAACTTGGTAATGGCAGATAAAAGCATCTTCTACTTTAGCTGCATGATGCAGTA | |
| CTTCCTGTCCTGCACTGCTGTGGTGACAGAGTCTTTCTTGGTGGCAGTGATGGCCTATGAC | |
| CGCTTTGTGGCCATCTGCAATCCTCTGCTTTATACAGTGGCCATGTCACAGAGGCTCTGTG | |
| CCCTGCTGGTGGCTGGGTCATATCTCTGGGGCATGTTTGGCCCCTTGGTACTCCTTTGTTAT | |
| GCTCTCCGGTTAAACTTCTCTGGACCTAATGTAATCAACCACTTCTTTTGTGAGTATACTGC | |
| TCTCATCTCTGTGTCTGGCTCTGATATACTCATCCCCCACCTGCTGCTTTTCAGCTTCGCCA | |
| CCTTCAATGAGATGTGTACACTACTGATCATCCTCACTTCCTATGTTTTCATTTTTGTGACT | |
| GTACTAAAAATCCGTTCTGTTAGTGGGCGCCACAAAGCCTTCTCCACCTGGGCCTCCCACC | |
| TGACTGCTATCACCATCTTCCATGGGACCATCCTTTTCCTTTACTGTGTACCCAACTCCAAA | |
| AACTCTCGGCAAACAGTCAAAGTGGCCTCTGTATTTTACACAGTTGTCAACCCCATGCTGA | |
| ACCCTCCGATCTACAGCCTAAGGAATAAAGACGTGAAGGATGCTTTCTGGAAGTTAATACA | |
| TACACAAGTTCCATTTCACTGA | |
| AOLFR3 sequences: | |
| MLLTDRNTSGTTFTLLGFSDYPELQVPLFLVFLAIYNVTVLGNIGLIVIIKINPKLHTPMYFFLSQ | (SEQ ID NO:5) |
| LSFVDFCYSSIIAPKMLVNLVVKDRTISFLGCVVQFFFFCTFVVTESFLLAVMAYDRFVAICNPL | |
| LYTVDMSQKLCVLLVVGSYAWGVSCSLELTCSALKLCFHGFNTINHFFCEFSSLLSLSCSDTYI | |
| NQWLLFFLATFNEISTLLIVLTSYAFIVVTILKMRSVSGRRKAFSTCASHLTAITIFHGTILFLYCV | |
| PNSKNSRIHTVKVASVFYTVVIPMLNPLIYSLRNKDVKDTVTEILDTKVFSY | |
| ATGCTGCTGACAGATAGAAATACAAGTGGGACCACGTTCACGCTCTTGGGCTTCTCAGATT | (SEQ ID NO:6) |
| ACCCAGAACTGCAAGTCCCACTCTTCCTGGTTTTTCTGGCCATCTACAATGTCACTGTGCTA | |
| GGGAATATTGGGTTGATTGTGATCATCAAAATCAACCCCAAACTGCATACCCCCATGTACT | |
| TTTTCCTCAGCCAACTCTCCTTTGTGGATTTCTGCTATTCCTCCATCATTGCTCCCAAGATG | |
| TTGGTGAACCTTGTTGTCAAAGACAGAACCATTTCATTTTTAGGATGCGTAGTACAATTCT | |
| TTTTCTTCTGTACCTTTGTGGTCACTGAATCCTTTTTATTAGCTGTGATGGCCTATGACCGC | |
| TTCGTGGCCATTTGCAACCCTCTGCTCTACACAGTTGACATGTCCCAGAAACTCTGCGTGC | |
| TGCTGGTTGTGGGATCCTATGCCTGGGGAGTCTCATGTTCCTTGGAACTGACGTGCTCTGC | |
| TTTAAAGTTATGTTTTCATGGTTTCAACACAATCAATCACTTCTTCTGTGAGTTCTCCTCAC | |
| TACTCTCCCTTTCTTGCTGTGATACTTACATCAACCAGTGGCTGCTATTCTTCTTGCCACC | |
| TTTAATGAAATCAGCACACTACTCATCGTTCTCACATCTTATGCGTTCATTGTTGTAACCAT | |
| CCTCAAGATGCGTTGAGTCAGTGGGCGCCGCAAAGCGTTCTCCACCTGTGCCTCCCACCTG | |
| ACTGCCATCACCATCTTCCATGGCACCATCCTCTTCCTTTACTGTGTGCCCTCTCCAAAAAA | |
| CTCCAGGCACACAGTCAAAGTGGCCTCTGTGTTTTACACCGTGGTGATCCCCATGTTGTTT | |
| CCCCTGATCTACAGTCTGAGAAATAAAGATGTCAAGGATACAGTCACCGAGATACTGGAC | |
| ACCAAAGTCTTCTCTTACTGA | |
| AOLFR4 sequences: | |
| MENQNVTEFILLGLTENLELWKTFSAVFLVMYVATVLENLLITTIITSQSLRSPMTFLTFLS | (SEQ ID NO:7) |
| LLDVMFSSVVAPKVIVDTLSKSTTISLKGCLTQLFVEHFFGGVGIILLTVMAYDRYVAICKPLHY | |
| TIIMSPRVCCLMVGGAWVGGFMHAMIQLLFMYQIPFCGPNIIDHFICDLFQLLTLACTDTHILGL | |
| LVTLNSGMMCVAIFLILIASYTVILCSLKSYSSKGRHKALSTCSSHLTVVVLFFVPCIFLYMRPV | |
| VTHPTDKAMAVSDSIITPMLNPLIYTLRNAEVKSAMXKLWMKWEALAGK | |
| ATGGAAAATCAAAACAATGTGACTGAATTCATTCTTCTGGGTCTCACAGAGTCCTGGAGC | (SEQ ID NO:8) |
| TGTGGAAAATATTTTCTGCTGTGTTTCTTGTCATGTATGTAGCCACAGTGCTGGAAAATCT | |
| ACTTATTGTGGTAACTATTATCACAAGTCAGAGTCTGAGGTCACCTATGTATTTTTTTCTTA | |
| CCTTCTTGTCCCTTTTGGATGTCATGTTCTCATCTGTCGTTGCCCCCTGGTGATTGTAGAC | |
| ACCCTCTCCAAGAGCACTACCATCTCTCTCAAAGGCTGCCTCACCCAGCTGTTGTGGAGC | |
| ATTTCTTTGGTGGTGTGGGGATCATCCTCCTCACTGTGATGGCCTATGACGGCTACGTGGC | |
| CATCTGTAAGCCCCTGCACTACACGATCATCATGAGTCCACGGGTGTGCTGCCTTTGGTA | |
| GGAGGGGCTTGGGTGGGGGGATTTATGCACGCAATGATACTCTTCTCTTCATGTATCAAA | |
| TACCCTTCTGTGGTCCTAATATCATAGATCACTTTATATGTGATTTGTTTCAGTTGTTGACA | |
| CTTGCCTGCACGGACACCCACATCCTGGGCCTCTTAGTTACCCTCTTCAGTGGGATGATGT | |
| GTGTGGCCATCTTTCTTATCTTAATTGCGTCCTACACGGTCATCCTATGCTCCCTGAAGTCT | |
| TACAGCTCTAAAGGGCGGCACAAAGCCCTCTCTACCTGCAGCTCCCACCTCACGGTGGTTG | |
| TATTGTTCTTTGTCCCCTGTATTTTCTTGTACATGAGGCGTGTGGTCACTCACCCCATAGAC | |
| AAGGCAATGGCTGTGTCAGACTCAATCATGACACCCATGTTTTATCCCTTGATCTATACAC | |
| TGAGGAATGCAGAGGTGAAAAGTGCCATGAAGAAACTCTGGATGTTGGGAGGCTTTGG | |
| CTGGGAAATAA | |
| AOLFR5 sequences: | |
| MGKENCTTVAEFILLGLSDVPELRVCLFLLFLLIYGVTLLANLGMIALIQVSSRLHTPMYFFLSH | (SEQ ID NO:9) |
| LSSVDFCYSSIIVPKMLANIFNKDKAISFLGCMVQFYLFCTCVVTEVFLLAVMAYDRFVAICNPL | |
| LYTVTMSWKVRVELASCCYFCGTVCSLIHLCLALRIPFYRSNVINHFFCDLPPVLSLACSDITVN | |
| ETLLFLVATLNESVTIMIILTSYLLILTTILKMGSAEGRHKAFSTCASHLTAITVFHGTVLSIYCRP | |
| SSGNSGDADKVATVFYTVVIPMLNSVIYSLRKDVKEALTTTGSKIHS | |
| ATGGGCAAGGAAAACTGCACCACTGTGGCTGAGTTCATTCTCCTGGACTATCAGATGTCC | (SEQ ID NO:10) |
| CTGAGTTGAGAGTCTGCCTCTTCCTGCTGTTCCTTCTCATCTATGGAGTCACGTGTTAGCC | |
| AACCTGGGCATGATTGCACTGATTCAGGTCAGCTCTCGGCTCCACACCCCCATGTACTTTT | |
| TCCTCAGCCACTTGTCCTCTGTAGATTTCTGCTACTCCTCAATAATGTGCCAAAAATGTTG | |
| GCTAATATCTTTAACAAGGACAAAGCCATCTCCTTCCTAGGGTGCATGGTGCAATTTCTACT | |
| TGTTTTGCACTTGTGTGGTCACTGAGGTCTTCCTGCTGGCCGTGATGGCCTATGACCGCTTT | |
| GTGGCCATCTGTAACCCTTTGCTATACACAGTCACCATGTCTTGGTGGTGCGTGTGGAGC | |
| TGGCTTCTTGCTGCTACTTCTGTGGGACGGTGTGTTCTCTGATTCATTTGTGCTTAGCTCTT | |
| AGGATCCCCTTCTATAGATCTAATGTGATTTCCACTTTTCTGTGATCTACCTCCTGTCT | |
| AAGTCTTGCTTGCTCTGATATCACTGTGAATGAGACACTGCTGTTCCTGGTGGCCACTTTG | |
| AATGAGAGTGTTACCATCATGATCATCCTCACCTCCTACCTGCTAATTCTCACCACCATCCT | |
| GAAGATGGGCTCTGCAGAGGGCAGGCACAAAGCCTTCTCCACCTGTGCTTCCCACCTCACA | |
| GCTATCACTGTCTTCCATGGAACAGTCCTTTCCAYITTAThTGCAGGCCCAGTTCAGGCTTA | |
| GTGGAGATGCTGACAAAGTGGCCACCGTGTTCTACACAGTCGTGATTCCTATGCTGTCTC | |
| TGTGATCTAGAGCCTGAGAAATAAAGATGTGAAAGAAGCTCTCAGTTTGTGATGGGCTC | |
| CAAAATTCACTCCTAG | |
| AOLFR6 sequences: | |
| MMASERNQSSTPTFILLGFSEYPEIQVPLFLVFLFVYTVTVVGNLGMIIIITNSTHTTTFLS | (SEQ ID NO:11) |
| HLSLTDFCFSTVVTPKTLENLVVEYRTISFSGCIMQFCFACIFGVTETFMLTTVAVCK | |
| PLLYTTIMSQKLCALLVAGSYTWGIVCSLILTTTLLDLSFCESTFINNFICDHSVIVSASYSDPYIS | |
| QRLCFITAIFNEVSSLIIILTSYMLIFTTIMKMRSASGRQKTFSTCASHLTAITIFHGTILFLYCTNP | |
| KTSSLIVTVASVFYTVAIPMLNPLIYSLRNKDLNNMFEKLVVTKLH | |
| ATGATGGCATCTGAAAGAAATCAAAGCAGCACACCCACTTTTATTCTCTTGGGTTTTCAG | (SEQ ID NO:12) |
| AATACCCAGAAATCCAGGTTCCACTCTTTCTGGTTTTCTTGTTCGTCTACACAGTCACTGTA | |
| GTGGGGAACTTGGGCATGATAATAATCATCAGACTCAATTCAATCTCCATACTTCATGT | |
| ACTTTTTCCTTAGTCACTTGTCCTTGACAGAGTTCTGTTTTTCCACTGTAGTTACACCTAT | |
| CTGTTGGAGAACTTGGTTGTGGAATACAGAACCATCTCTTTCTCTGGTTGCATCATGCTT | |
| TTTGTTTTGCTTGCATTTTTGGAGTGACAGAAACTTTCATGTTAGCAGCGATGGCTTATGAC | |
| CGTTTTGTGGCAGTTTGTAAACCCTTGCTGTATACCACTATTATGTCTCAGTGCTCTGTGC | |
| TCTTCTGGTGGCTGGGTCCTATACATGGGGGATAGTGTGCTCCCTGATACTCACATATTTT | |
| CTTCTTGACTTATCGTTTTGTGAATCTACCTTCATAAATAATTTTATCTGTGACCACTCTGT | |
| AATTGTTTCTGCCTCCTACTCAGACCCCTATATCAGCCAGAGGCTATGCTTTATTATTGCCA | |
| TATTCAATGAGGTGAGCAGCCTAATTATCATTCTGACATCATATATGCTTATTTTCACTACC | |
| ATTATGAAGATGCGATCTGCAAGTGGGCGCCAGAAAACTTTCTCCACCTGTGCCTCCCACC | |
| TGACAGCCATCACTATCTTCCATGGAACTATCCTTTTCCTTTACTGTGTTCCTTTCCTAT | |
| ACTTCTAGCCTCATAGTTACAGTGGCTTCTGTGTTTTACACAGTGGCGATTCCAATGCTGA | |
| ACCCATTGATCTACAGCCTTAGGAACAAAGATATCAATAACATGTTTGAAAAATTAGTGT | |
| CAGCAAATTGATTTACCACTGA | |
| AOLFR7 sequences: | |
| MSYFYRLKLMKEAVLVKLPFTSLPLLLQTLSRKSRDMEITYSSSTSGFILLGLSSTQLQKPLF | (SEQ ID NO:13) |
| AIFLIMYLLAAVGNVLIIPAIYSDPRLHTPMTTFLSNLSFMDICVFTVIVPKMLVNFLSETKVISY | |
| VGCLAQMTTFMAIFGNTDSYLLASMAIDRLVAICNPLHYDVVMKPRHCLLMLLGSCSISHLHSL | |
| FRVLLMSRLSFCASHIIKHFFCDTQPVLKLSCSDTSSSQMVVMTETLAVIVTPFLCIIFSYLRIMV | |
| TVLRIPSAAGKWKAFSTCGSHLTAVALFYGSIIYVTTRPLSMYSVVRDRVATVMYTTTPMLN | |
| PFIYSLRNKDMKRGLKKLQDRIYR | |
| ATGAGCTATTTTTACAGGCTTAAGCTTATGAAAGAAGCTGTCTTGGTCAAACTGCCCTTTA | (SEQ ID NO:14) |
| CATCTCTCCCACTGCTTCTCCAAACCCTATCCAGGAAGTCCAGAGACATGGAGATTGT | |
| CTACAGCAGCAGCACCTCAGGCTTCATCCTCCTGGGCCTCTCTTCCAACCCTCAGCTGCAG | |
| AAACCTCTCTTTGCCATCTTCCTCATCATGTACCTGCTCGCTGCGGTGGGGAATGTGCTGAT | |
| CATCCCGGCCATCTACTCTGACCCCAGGCTCCACACCCCTATGTACTTTTTTCTCAGCAACT | |
| TGTCTTTCATGGATATCTGCTTCACAACAGTCATAGTGCCTAAGATGCTGGTGTTTTCTA | |
| TCAGAGACAAAGGTTATCTCCTATGTGGGCTGCCTGGCCCAGATGTACTTCTTTATGGCAT | |
| TTGGGAACACTGACAGCTACCTGCTGGCCTCTATGGCCATCGACCGGCTGGTGGCCATCTG | |
| CAACCCCTTACACTATGATGTGGTTATGAAACCACGGCATTGCCTGCTCATGCTATTGGGT | |
| TCTTGCAGCATCTCCCACCTACATTCCCTGTTCCGCGTGCTACTTATGTCTCGCTTGTCTTT | |
| CTGTGCCTCTCACATCATTAAGCACTTTTTCTGTGACACCCAGCCTGTGCTAAAGCTCTCCT | |
| GCTCTGACACATCCTCCAGCCAGATGGTGGTGATGACTGAGACCTTAGCTGTCATGTGAC | |
| CCCCTTCCTGTGTATCATCTTCTCCTACCTGCGAATCATGGTCACTGTGCTCAGAATTCCCCT | |
| CTGCAGCCGGGAAGTGGAAGGCCTTCTCTACCTGTGGCTCCCACCTCACTGCAGTAGCCCT | |
| TTTCTATGGGAGTATTATTTATGTCTATTTTAGGCCCCTGTCCATGTACTCAGTGGTTAGGG | |
| ACCGGGTAGCCACAGTTATGTACACAGTAGTGACACCCATGCTGAACCCTTPCATCTACAG | |
| CCTGAGGAACAAAGATATGAAGAGGGGTTTGAAGAAATTACAGGACAGAATTTACCGGTA | |
| A | |
| AOLFR8 sequences: | |
| MATSNHSSGAEFILAGLTQRPELQLPLFLLFLGIYVVTVVGNLGMIFLIALSSQLYPPVYYFLSH | (SEQ ID NO:15) |
| LSFIDLCYSSVITPKMLVNFVPEENIISFLECITQLTTFLIFVIAEGYLLTAMEYDRYVAICRTPLLY | |
| NIVMSHRVCSIMMAVVYSLGFLWATVHTTRMSVLSFCRSHTVSHTTCDILPLLTLSCSSTHTNEI | |
| LLFIIGGVNTLATTLAVLISYAFIFSSILGIHSTEGQSKAFGTCSSHLLAVGIFFGSITFMTTKPPSS | |
| TTMEKEKVSSVFYITIIPMLNPLIYSLRNKDVKNALKKMTRGRQSS | |
| ATGGCTACTTCAAACCATTCTTCAGGGGCTGAGTTTATCCTGGCAGGCTTGACACAACGCC | (SEQ ID NO:16) |
| CAGAACTTCAACTGCCACTCTTCCTCCTGTTCCTTGGAATATATGTGGTCACAGTGGTGGG | |
| GAACCTGGGCATGATCTTCTTAATTGCTCTCAGTTCTCAACTTTACCCTCGAGTGTATTATT | |
| TTCTCAGTCATTTGTCTTTCATTGATCTCTGCTACTCCTCTGTCATTACCCCTAAGATGCTG | |
| GTGAACTTTGTTCCAGAGGAGAACATTATCTCCTTTCTGGAATGCATTACTCAACTTTATTT | |
| CTTCCTTATTTTTGTAATTGCAGAAGGCTACCTTCTGACAGCCATGGAATATGACCGTTAT | |
| GTTGCTATCTGTCGCCCACTGCTTTACAATATTGTCATGTCCCACAGGGTCTGTTCCATAAT | |
| GATGGCTGTGGTATACTCACTGGGTTTTCTGTGGGCCACAGTCCATACTACCCGCATGTCA | |
| GTGTTGTCATTCTGTAGGTCTCATACGGTCAGTCATTATTTTTGTGATATTCTCCCCTTATT | |
| GACTCTGTCTTGCTCCAGCACCCACATCAATGAGATTCTGCTGTTCATTATTGGAGGAGTT | |
| AATACCTTAGCAACTACACTGGCGGTCCTTATCTCTTATGCTTTCATTTTCTCTAGTATCCT | |
| TGGTATTCATTCCACTGAGGGGCAATCCAAAGCCTTTGGCACTTGTAGCTCCCATCTCTTG | |
| GCTGTGGGCATCTTTTTTGGGTCTATAACATTCATGTATTTCAAGCCCCCTTCCAGCACTAC | |
| TATGGAAAAAGAGAAGGTGTCTTCTGTGTTCTACATCACAATAATCCCCATGCTGAATCCT | |
| CTAATCTATAGCCTGAGGAACAAGGATGTGAAAAATGCACTGAAGAAGATGACTAGGGGA | |
| AGGCAGTCATCCTGA | |
| AOLFR9 sequences: | |
| MLARNNSLVTEFILAGLTDRPEFWQPFFFLFLVIYIVTMVGNLGLITLFGLNSHLHTPMYYFLFN | (SEQ ID NO:17) |
| LSFIDLCYSSVFTPKMLMNFVSKKNIISNVGCMTRLFFFLFFVISECYMLTSMAYDRYVAICNPL | |
| LYKVTMSHQVCSMLTFAAYIMGLAGATAHTGCMFRLTFCSANIINHYLCDILPLLQLSCTSTYV | |
| NEVVVLIVVGTNITVPSCTILISYVFIVTSILHIIKSTQGRSKAFSTCSSHVIALSLFFGSAAFMYIKY | |
| SSGSMEQGKVFSVFYTNVVPMLNPLIYSLRNKDVKVALRKTALIKIQRRNIF | |
| ATGCTGGCTAGAAACAACTCCTTAGTGACTGAATTTATTCTTGCTGGATTAACAGATCGTC | (SEQ ID NO:18) |
| CAGAGTTCTGGCAACCCTTCTTTTTCCTGTTCCTAGTGATCTACATTGTCACCATGGTAGGC | |
| AACCTTGGCTTGATCACTCTTTTCGGTCTAAATTCTCACCTCCACACACCAATGTACTATTT | |
| CCTCTTCAATCTCTCCTTCATTGATCTCTGTTACTCGTCTGTTTTGACTCCCAAAATGCTAAT | |
| GAACTTTGTGTCAAAAAAGAATATTATCTCCAATGTTGGGTGCATGACTCGGCTGTTTTTC | |
| TTTCTCTTTTTCGTCATCTCTGAATGTTACATGTTGACCTCAATGGCATATGATCGCTATGT | |
| GGCCATCTGTAATCCATTGCTGTATAAGGTCACCATGTCCCATCAGGTCTGTTCTATGCTCA | |
| CTTTTGCTGCTTACATAATGGGATTGGCTGGAGCCACGGCCCACACCGGGTGCATGTTTAG | |
| ACTCACCTTCTGCAGTGCTAATATCATTAACCATTACTTGTGTGACATACTCCCCCTCCTCC | |
| AGCTTTCCTGCACCAGCACCTATGTCAACGAGGTGGTTGTTCTCATTGTTGTGGGTACTAA | |
| TATCACGGTACCCAGTTGTACCATCCTCATTTCTTATGTTTTCATTGTCAGTAGCATTCTTC | |
| ATATCAAATCCACTCAAGGAAGATCAAAAGCCTTCAGTACTTGTAGCTCTCATGTCATTGC | |
| TCTGTCTCTGTTTTTTGGGTCAGCGGCATTCATGTATATTAAATATTCTTCTGGATCTATGG | |
| AGCAGGGAAAAGTTTTTTCTGTTTTCTACACTAATGTGGTGCCCATGCTCAATCCCCTCATC | |
| TACAGTTTGAGGAACAAGGATGTCAAAGTTGCACTGAGGAAAGCTCTGATTAAAATTCAG | |
| AGGAGAAATATATTCTAA | |
| AOLFR10 sequences: | |
| MLARNNSLVTEFILAGLTDRPEFRQPLFFLFLVIYIVTMVGNLGLIILFGLNSHLHTPMYTTLFNL | (SEQ ID NO:19) |
| SFIDLCYSSVFTPKMLMNFVSKKNIISYVGCMTQLFFFLFFVISEGYILTSMAYDRYVAICNPLLY | |
| KVTMSHQVCSMLTFAAYIMGLAGATAHTGCMLRLTFCSANIINHYLCDILPLLQLSCTSTYVN | |
| EVVVLIVVGINIMVPSCTILISYVFIVTSILHIKSTQGRSKAFSTCSSHVIALSLFFGSAAFMYIKYS | |
| SGSMEQGKVSSVEYTNVVPMLNPLIYSLRNKDVKVALRLKALIKIQRRNIF | |
| ATGCTGGCTAGAAACAACTCCTTAGTGACTGAATTTATTCTTGCTGGATTAACAGATCGTC | (SEQ ID NO:20) |
| CAGAGTTCCGGCAACCCCTCTTTTTCCTGTTTCTAGTGATCTACATTGTCACCATGGTAGGC | |
| AACCTTGGCTTGATCATTCTTTTCGGTCTAAATECTCACCTCCACACACCAATGTACTATTT | |
| CCTCTTCAATCTCTCCTTCATTGATCTCTGTTACTCCTCTGTTTTCACTCCCAAAATGCTAAT | |
| GAACTTTGTATCAAAAAAGAATATTATCTCCTATGTTGGGTGCATGACTCAGCTGTTTTTCT | |
| TTCTCTTTTTTGTCATCTCTGAATGCTACATATTGACCTCTTGGCATATGATCGCTATGTG | |
| GCCATCTGTAATCCATTGCTGTATAAGGTCACCATGTCCCATCAGGTCTGTTCTATGCTCAC | |
| TTTTGCTGCTTACATAATGGGATTGGCTGGAGCCACGGCCCACACCGGGTGCATGCTTAGA | |
| CTCACCTTCTGCAGTGCTAATATCATCAACCATTACTTGTGTGACATACTCCCCCTCCTCCA | |
| GCTTTCCTGCACCAGCACCTATGTCAACGAGGTGGTTGTTCTCATTGTTGTGGGTATTTT | |
| ATCATGGTACCCAGTTGTACCATCCTCATTTCTTATGTTTTCATTGTCACTAGCATTCTTCA | |
| TATCAAATCCACTCAAGGAAGATCAAAAGCCTTCAGTACTTGTAGCTCTCATGTCATTGCT | |
| CTGTCTCTGTTTTTTGGGTCAGCGGCATTCATGTATATTAAATATTCTTCTGGATCTATGGA | |
| GCAGGGAAAAGTTTCTTCTGTTTTCTACACTAATGTGGTGCCCATGCTCTTCCTCTCATCT | |
| ACAGTTTGAGGAACAAGGATGTCAAAGTTGCACTGAGGAGCTCTGATTTTTTTCAGA | |
| GAAGAAATATATTCTAA | |
| AOLFR11 sequences: | |
| MTLRNSSSVTEFILVGLSEQPELQLPLFLLFLGIYVFTVGNLGLITLIGTSLHTPMTFLFNLS | (SEQ ID NO:21) |
| FIDLCYSCVFTPKMLNDFVSESIISYVGCMTQLFFFCFFVNSECYVLVSMAYDRYVAICNPLLY | |
| MVTMSPRVCFLLMFGSYVVGFAGAMAHTGSMLRLTFCDSNVIDHTCDVLPLLQLSCTSTHV | |
| SELVFFIVVGVITMLSSISIVISYALILSNILCIPSAEGRSKAFSTWGSHIIAVALFFGSGTFTYLTTS | |
| FPGSMHGRFASVFYTNVVPMLNPSIYSLRNKDDKLALGKTLTVLF | |
| ATGACTCTGAGAAACAGCTCCTCAGTGACTGAGTTTATCCTTGTGGGATTATCAGTCAGC | (SEQ ID NO:22) |
| CAGAGCTCCAGCTCCCTCTTTTCCTTCTATTCTTAGGGATCTATGTGTTCACTGTGGTGGGC | |
| AACTTGGGCTTGATCACCTTAATTGGGATAAATCCTAGCCTCACACCCCCATGTACTTTTT | |
| CCTCTTCAACTTGTCCTTTATAGATCTCTGTTATTCCTGTGTGTTTACCCCCTTTTGCTGA | |
| ATGACTTTGTTTCAGAAAGTATCATCTCTTATGTGGGATGTATGACTCAGCTATTTTTCTTC | |
| TGTTTCTTTGTCAATTCTGAGTGCTATGTGTTGGTATCTTGGCCTATGATCGCTATGTGGC | |
| CATCTGCAACCCCCTGCTCTACATGGTCACCATGTCCCCAAGGGTCTGCTTTCTGCTGATGT | |
| TTGGTTCCTATGTGGTAGGGTTTGCTGGGGCCATGGCCCACACTGGAAGCATGCTGCGACT | |
| GACCTTCTGTGATTCCAACGTCATTGACCATTATCTGTGTGACGTTCTCCCCCTCTTGCAGC | |
| TCTCCTGCACCAGCACCCATGTCAGTGAGCTGGTATTTTTCATTGTTGTTGGAGTTTCACC | |
| ATGCTATCCAGCATAAGCATCGTCATCTCTTACGCTTTGATACTCTCCTCATCCTCTGTAT | |
| TCCTTCTGCAGAGGGCAGATCCAAAGCCTTTAGCACATGGGGCTCCCACATTTTGCTGTT | |
| GCTCTGTTTTTTGGGTCAGGGACATTCACCTACTTAACAACATCTTTTCCTGGCTCTATGT | |
| CCATGGCAGATTTGCCTCAGTCTTTTACACCAATGTGGTTCCCATGCTTTCCCTTCGATCT | |
| ACAGTTTGAGGAATAAGGATGATAAACTTGCCCTGGGCAATTCCCTGTGAGAGTGCTCT | |
| TCTAA | |
| AOLFR12 sequences: | |
| MERNHNPDNCNVLNFFFADKKNKRRFGQIVSDVGPICYSVSLSLGEPTTMGTNLTITSEFIL | (SEQ ID NO:23) |
| LGLSSRPEDQKPLFAVFLPIYLITVIGNLLIILATSDTRTQTPMTTFLSILSFVDICTTVIIPKMLV | |
| NFLSETKTISYGECLTQMTTFLAFGNTDSYLLAAMAIDRYVAICNPFHYITIMSHRCCTLLVLS | |
| FCIPHFHSLLHILLTNQLIFCASNVIHHFFCDDQPVLKLSCSSHFVKEITTTEGLAVNTPFSCIII | |
| SYLRILITVLKIPSAAGKRKFSTCGSHLTVVTLFYGSISYVTTQPLSNYTTIIQIATIIYTVLTP | |
| MLNPFIYSLRNKDMKQGLAKLMHRMKCQ | |
| ATGGAAAGAAACGACAATCCAGATAATTGTAATGTTTTAAATTTTTTCTTGCTGATTGA | (SEQ ID NO:24) |
| AGAATAAAAGGAGAAATTTTGGACAGATTGTATCAGATGTTGGAAGTTTCTGTTACAGTG | |
| TTAGTTTATCTTTAGGTGAACCCACAACTATGGGAAGAAATAACCTCTGACCCTCTGA | |
| ATTCATCCTCCTTGGAGTCTCCTCTCGACCTGAGGATCAGAAGCCGCTCTTTGCTGTGTTCC | |
| TCCCCATCTACCTTATCACAGTGATAGGAAACCTGCTTATCATCCTGGCCATCCGCTCAGA | |
| CACTCGTCTCCAGACGCCCATGTACTTCTTTCTAAGCATCCTGTCTTTGTTGAGATTTGCT | |
| ATGTGACAGTCATTATCCCTAAGATGCTGGTGAACTTCTTATCAGAGACAAAGACCATCTC | |
| TTACGGTGAGTGTCTGACCCAGATGTACTTTTTCTTAGCCTTTGGTCACAGACAGTAC | |
| CTGCTAGCAGCCATGGCCATTGACCGCTATGTGGCCATATGTTTCCCTTCCACTACATCA | |
| CCATTATGAGTCACAGATGCTGTGTCCTGCTTCTGGTTCTCTCCTTCTGCATTCCACATTTT | |
| CACTCCCTCCTGCACATTCTTCTGACTAATCAGCTCATCTTCTGTGCCTCCTTGTCATCCA | |
| TCACTTTTTCTGCGATGATCAACCAGTGCTAATTGTCCTGTTCCTCCCATTTGTCATG | |
| AAATCACAGTAATGACAGAAGGCTGGCTGTCATAATGACCCCGTTTTCATGCATCATCAT | |
| CTCTTATTTAAGAATCCTCATCACTGTTCTGAAGATTCCTTCAGCTGCTGGAAAGCGTAAA | |
| GCATTTTCTACCTGTGGCTCTCATCTCACAGTGGTGACCCTGTTTTATGGAAGCATTAGCTA | |
| TGTCTATTTTCAGCCCCTGTCCAACTATACTGTCAAGGATCAAATAGCAACAATTATCTAC | |
| ACCGTACTGACTCCTATGCTAAATCCATTTATCTATAGTCTGAGGAACAAAGACATGAAGC | |
| AGGGTTTGGCAAAGTTGATGCACAGGATGAAATGTCAGTAA | |
| AOLFR13 sequences: | |
| MDQKNGSSFTGFILLGFSDRPQLELVLFVVLLIEYIFTLLGNKTIIVLSHLDPHLHNPMTTFFSNL | (SEQ ID NO:25) |
| SFLDLCYTTGTVPQLLVKLRGADKSISYGGCVVQLYISLGLGSTECVLLGVMAFDRYAAVCRPL | |
| HYTVVMHPCLYVLMASTSWVIGFANSLLQTVLILLLTLCGRNKLEHFLCEVPPLLKTACVDTT | |
| MNESELFFVSVIILLVPVALIIFSYSQIVRAVVRIKSATGQRXVFGTCGSHLTVVSLFYGTAIYAY | |
| LQPGNNYSQDQGKXISLFYTIITPMLNPLIYTLRNKDVKGALKKVLWKNYDSR | |
| ATGGATCAGAAAAATGGAAGTTCTTTCACTGGATTTATCCTACTGGGTTTCTCTGACAGGC | (SEQ ID NO:26) |
| CTCAGCTGGAGCTAGTCCTCTTTGTGGTTcTTTFITGATCTTCTATATCTTCACTTTGCTGGGG | |
| AACAAAACCATCATTGTATTATCTCACTTGGACCCACATCTTCACAATCCTATGTATTTTTT | |
| CTTCTCCAACCTAAGCTTTTTGGATCTGTGTTACACAACCGGCATTGTTCCACAGCTCCTGG | |
| TTAATCTCAGGGGAGCAGACAAATCAATCTCCTATGGTGGTTGTGTAGTTCAGCTGTACAT | |
| CTCTCTAGGCTTGGGATCTACAGAATGCGTTCTCTTAGGAGTGATGGCATTTGACCGCTAT | |
| GCAGCTGTTTGCAGGCCCCTCCACTACACAGTAGTCATGCACCCTTGTCTGTATGTGCTGA | |
| TGGCTTCTACTTCATGGGTCATTGGTTTTGCCAACTCCCTATTGCAGACGGTGCTCATCTTG | |
| CTTTTAACACTTTGTGGAAGAAATAAATTAGAACACTTTCTTTGTGAGGTTCCTCCATTGCT | |
| CAAGCTTGCCTGTGTTGACACTACTATGAATGAATCTGAACTCTTCTTTGTCAGTGTCATTA | |
| TTCTTCTTGTACCTGTTGCATTAATCATATTCTCCTATAGTCAGATTGTCAGGGCAGTCGTG | |
| AGGATAAAGTCAGCAACAGGGCAGAGAAAAGTGTTTGGGACATGTGGCTCCCACCTCACA | |
| GTGGTTTCCCTGTTCTACGGCACAGCTATCTATGCTtACCTCCAGCCCGGCAACAACTACTC | |
| TCAGGATCAGGGCAAGKTCATCTCTCTCTFTCTACACCATCATTACACCCATGATCAACCCC | |
| CTCATATATACACTGAGGAACAAGGATGTGAAAGGAGCACTTAAGAAGGTGCTCTGGAAG | |
| AACTACGACTCCAGATGA | |
| AOLFR14 sequences: | |
| MALPLLLSPSCFASSQSLSSRMNSENLTRAAVAPAEFVLLGITNRWDLRVALFLTCLPVYLVSL | (SEQ ID NO:27) |
| LGNMGMALLIRMDARLHTPMTTFLANLSLLDACYSSAIGPKMLVDLLLPRATIPYTACALQMF | |
| VFAGLADTECCLLAAMAYDRYVAIRNPLLYTTAMSQRLCLALLGASGLGGAVSAFVHIFPLTF | |
| RLSFCRSRKINSFFCDIPPLLAISCSDTSLNELLLFAICGFIQTATVLAITVSYGFIAGAVIHMRSVE | |
| GSRIRAASTGGSHLTAVAMMYGTLIFMYLRPSSSYALDTDKMASVFYTLVIPSLNPLIYSLRNKE | |
| VKEALRQTWSRFHCPGQGSQ | |
| ATGGCCTTGCCATTGCTCTTATCTCCCTCCTGCTTTGCCTCTTCTCAGTCTCTGTCCAGTAG | (SEQ ID NO:28) |
| GATGAAGTCAGAGAACCTCACCCGGGCCGCGGTTGCCCCTGCTGAATTCGTCCTCCTGGGC | |
| ATCACAAATCGCTGGGACCTGCGTGTGGCCCTCTTCCTGACCTGCCTGCCTGTCTACCTGG | |
| TGAGCCTGCTGGGAAACATGGGCATGGCGCTGCTGATCCGCATGGATGCCCGGCTCCACA | |
| CACCTATGTACTTCTTCCTGGCCAACCTCTCCCTGCTGGATGCCTGCTATTGCTCCGCCATC | |
| GGCCCCAAGATGCTAGTGGACCTGCTGCTGCCCCGAGCCACCATCCCTTACACAGCCTGTG | |
| CCCTCCAGATGTTTGTCTTTGCAGGTCTGGCTGATACTGAGTGTTGCTTGCTGGCAGCCAT | |
| GGCCTATGACCGCTACGTGGCCATCAGAAACCCACTTCTCTATACAACAGCTATGTCGCAG | |
| CGTCTATGCCTGGCCTTGCTGGGAGCATCAGGCCTGGGTGGGGCAGTGAGTGCCTTTGTTC | |
| ACACAACCCTCACCTTCCGCCTGAGCTTCTGCCGCTCCCGGAAGATCAATAGCTTCTTCTG | |
| CGATATCCCTCCACTGCTGGCCATCTCGTGCAGTGACACCAGTCTGAATGAACTCCTTCTCT | |
| TCGCCATGTGTGGCTTCATCCAGACAGCCACGGTGTTAGCTATCACGGTGTCTTATGGCTT | |
| CATCGCTGGGGCTGTGATCCACATGCGCTCGGTCGAGGGCAGTCGGCGAGCAGCCTCCAC | |
| CGGTGGTTCCCACCTCACAGCCGTGGCCATGATGTACGGGACACTCATTTTCATGTACCTG | |
| CGCCCCAGCTCCAGCTATGCCCTGGACACTGACAAGATGGCCTCTGTGTTCTATACCCTGG | |
| TCATCCCGTCTCTCTCCCACTCATCTACAGCCTCCGCTTITGGAGGTCTGGAGGCCCT | |
| CAGGCAGACCTGGAGCCGATTCCACTGTCCAGGGCAGGGGTCCCAGTGA | |
| AOLFR15 sequences: | |
| MRENNQSSTLEFILLGVTGQQEQEDFFYILFLFIYPITLIGNLLIVLAICSDVRIHNPMTTLLANLS | (SEQ ID NO:29) |
| LVDIFFSSVTIPKMLANHLLGSKSISFGGCLTQMTTMIALGNTDSYILTlTVAISHPLH | |
| YTTIMSPRSCIWLIAGSWVIGNANALPHTASLSCGNQEVANFYCDITPLLKLSCSDIHFHV | |
| KMMYLGVGIFSVPLLCIIVSYIRVFSTVFQVPSTKGVLTFSTCGSHLTVVSLYYGTTGTYFR | |
| PLTNYSLKDAVITVMYTAVTPMLNPFIYSLRNRDMKAALRKLTTTTISS | |
| ATGAGGGAAAATAACCAGTCCTCTACACTGGAATTCATCCTCCTGGGAGTACTGGTCAGC | (SEQ ID NO:30) |
| AGGAACAGGAAGATTTCTTCTACATCCTCTTCCTGTTCATTTACCCCATCACATTGATTGGA | |
| AACGTGCTCATTGTCCTAGCCATTGCTCTGATGTTCGCCTTCACTTCCCCATGTATTTCT | |
| CCTTGCCAACCTCTCCTTGGTTGACATCTTCTTCTCATCGGTAACCATCCCTTGATGCTGG | |
| CCAACCATCTCTTGGGCAGCAAATCCATCTCTTTTGGGGGATGCCTTCGCAGATGTATTT | |
| CATGATAGCCTTGGGTAACACAGACAGCTATTTTGGCTGCAATGGCATATGATCGAGCT | |
| GTGGCCATCAGCCACCCACTTCACTACACAACAATTATGAGTCCACGGTCTTGTATCTGGC | |
| TTATTGCTGGGTCTTGGGTGATTGGAAATGCCAATGCCCTCCCCCACACTCTGCTCACAGC | |
| TAGTCTGTCCTTCTGTGGCAACCAGGAAGTGGCCAACTTGTACTGTGACATTACCCCCTTG | |
| CTGAAGTTATCCTGTTCTGACATCCACTTTCATGTGAAGATGATGTACCTAGGGGTTGGCA | |
| TTTTCTCTGTGCCATTACTATGCATCATTGTCTCCTATATTCGAGTCTTCTCCACAGTCTTCC | |
| AGGTCCTTCCACCAAGGGCGTGCTCAAGGCCTTCTCCACCTGTGGTTCCCACCTCACGGT | |
| TGTCTCTTTGTATTATGGTACAGTCATGGGCACGTATTTCCGCCCTTTGACCAATATAGCC | |
| TAAAAGACGCAGTGATCACTGTAATGTACACGGCAGTGACCCCTTGTTATTCCTTTCAT | |
| CTACAGTCTGAGAATCGGGAGATGAAGGCTGCCCTGCGGAAACTCTTCTCTGAGAAT | |
| CTCCTCGTAA | |
| AOLFR16 sequences: | |
| MRRNCTLVTEFILLGLTSRRELQILLFTLFLAIYMVTVAGNLGMIVLIQANAWLIIMPMYFFLSH | (SEQ ID NO:31) |
| LSFVDLCFSSNVTPKMLEWLSEKKSISYPACLVQCYLFIALVHVEIYILAVMAFDRYMAICNPLL | |
| YGSRMSKSVCSFLITVPYVYGALTGLMETMWTYNLAFCGPNEINHFYCADPPLITACSDTYN | |
| KELSMFIVAGWNLSFSLFIICISYLYIFPAILKIRSTEGRQKAFSTCGSHLTAVTWYATLFFMYLR | |
| PPSKESVEQGKMVAVFYTTVIPMLNLIIYSLPTNKNVKEALIKELSMKIYFS | |
| ATGAGAAGAAACTGCACGTTGGTGACTGAGTTCATTCTCCTGGGACTGACCAGTCGCCGG | (SEQ ID NO:32) |
| GAATTACAAATTCTCCTCTTCACGCTGTTTCTGGCCATTTACATGGTCACGGTGGCAGGGA | |
| ACCTTGGCATGATTGTCCTCATCCAGGCCAACGCCTGGCTCCACATGCCCATGTACTTTTTC | |
| CTGAGCCACTTATCCTTCGTGGATCTGTGCTTCTCTTCCAATGTGACTCCATGATGCTGG | |
| AGATTTTCCTTTCAGAGAAGAAAAGCATTTCCTATCCTGCCTGTGTTGTGCAGTGTTACCTT | |
| TTTATCGCCTTGGTCCATGTTGAGATCTACATCCTGGCTGTGATGGCCTTTGACCGGTACAT | |
| GGCCATCTGCAACCCTCTGCTTTATGGCAGCAGAATGTCCAAGAGTGTGTGCTCCTTCCTC | |
| ATCACGGTGCCTTATGTGTATGGAGCGCTCACTGGCCTGATGGAGACCATGTGGACCTACA | |
| ACCTAGCCTTCTGTGGCCCCAATGAAATTAATCACTTCTACTGTGCGGACCCACCACTGAT | |
| TAAGCTGGCTTGTTCTGACACCTACAAGAAGGAGTTGTCAATGTTTATTGTGGCTGGCTGG | |
| AACCTTTCTTTTTCTCTCTTCATCATATGTATTTCCTACCTTTACATTTTCCCTGCTATTTTA | |
| AAGATTCGCTCTACAGAGGGCAGGCAAAAAGCTTTTTCTACCTGTGGCTCCCATCTGACAG | |
| CTGTCACTATATTCTATGCAAGCCTTTTCTTCATGTATCTCAGACCCCCCTCAAAGGAATCT | |
| GTTGAACAGGGTAAAATGGTAGCTGTATTTTATACCACAGTAATCCCTATGCTGAACCTTA | |
| TAATTTATAGCCTTAGAAATAAAAATGTAAAAGAAGCATTAATCAAAGAGCTGTCAATGA | |
| AGATATACTTTTCTTAA | |
| AOLFR17 sequences: | |
| MLNFTDVTEFILLGLTSRREWQVLFFIIFLVVYIITMVGNIGMMVLIKVSPQLNNPMTTFLSHLS | (SEQ ID NO:33) |
| FVDVWFSSNVTPKMLENLFSDKKTITYAGCLVQCFFFIALVHVEIFILAAMAFDRYMAIGNPLL | |
| YGSKMSRVVCIRLITFPYIYGFLTSLAATLWTYGLTTCGKIEINHFYCADPPLIKMACAGTFVKE | |
| YTMIILAGTNTTYSLTVIIISYLFILIAILRMRSAEGRQKAFSTCGSHLTAVIWYGTLIFMYLRRPTE | |
| ESVEQGKMVAVFYTTVIPMLNPMIYSLRNKDVKKAMMKVISRSC | |
| ATGCTCAATTFFCACCGATGTGACAGAGTTCATTCTTTTGGGGCTAACGAGCCGTCGAGTT | (SEQ ID NO:34) |
| GGCAAGTTCTCTTCTTCATCATCTTTCTTGTGGTCTACATCATCACCATGGTGGGCAATATG | |
| GGCATGATGGTGTTAATCAAGGTCAGTCCTCAGCTTAACAACCCCATGTACTTTTCCTCA | |
| GTCACTTGTCATTTGTTGATGTGTGGTTTTCTTCCAATGTCACCCCTTGTTGGTTTC | |
| CTGTTTTCAGATAAAAAAACAATTACTTATGCTGGTTGTTTAGTACAGTGTTTCTTCTTCAT | |
| TGCTCTTGTCCATGTGGAAATTTTTATTGTTGCTGCGATGGCCTTTGATAGATACATGGCTT | |
| TTGGGAATCCTCTGCTTTATGGCAGTAAAATGTCAAGGGTFGTCTGTATTCGACTGATTAC | |
| TTTCCCTTACATTTATGGTTTTCTGACGAGTCTGGCAGCAACATTATGGACTTACGGCTTGT | |
| ACTTCTGTGGAAAATTGAGATCAACCATTTCTACTGTGCAGATCCACCTCTCATCTTTT | |
| GGCCTGTGCCGGGACCTTTGTAAAAGAATATACAATGATCATACTTGCCGGCATTTCTTC | |
| ACATATTCCCTGACTGTAATTATCATCTCTTACTTATTCATCCTCATTGCCATTCTGCGTT | |
| GCGCTCAGCAGAAGGAAGGCAGAAGGCCTTTTCCACATGTGGGTCCCATCTGACAGCTGT | |
| CATTATATTCTATGGTACTCTGATCTTCATGTATCTCAGACGTCCCACAGAGGAGTCTGTG | |
| GAGCAGGGGAAGATGGTGGCTGTGTTCTATACCACAGTGATCCCCATGTTGTTGCCATGA | |
| TCTACAGTCTGAGGAACAAGGATGTGAAAAAGGCCATGATGAAAGTGATCAGCAGATCAT | |
| GTTAA | |
| AOLFR18 sequences: | |
| MSNTNGSAITEFILLGLTDCPELQSLLFVLFLVVYLVTLLGNLGMIMLMRLDSRLHTPMTFLT | (SEQ ID NO:35) |
| NLAFVDLCYTSNATPQMSTNIVSEKTISFAGCFTQCYWALLLTEFYMLAAMAYDRYVAIYDP | |
| LRYSVKTSRRVCICLATFPYYGFSDGLFQAILTFRLTFCRSNVNHFYCADPPLIKLSCSDTYVK | |
| EHAMFISAGFNLSSSLTIVLVSYAFILAAILRIKSAEGRHKAFSTCGSHMMAVTLFYGTLFCMYI | |
| RPPTDKTVEESKIIAVFYTFVSPVLNPLIYSLRNXDVKQALKNVLR | |
| ATGTCCAACACAAATGGCAGTGCAATCACAGAATTCATTTTACTTGGGCTCACAGATTGCC | (SEQ ID NO:36) |
| CGGAACTCCAGTCTCTGCTTTTTGTGCTGTTTCTGGTTGTTTACCTCGTCACCCTGCTAGGC | |
| AACCTGGGCATGATAATGTTAATGAGACTGGACTCTCGCCTTCACACGCCCATGTACTTCT | |
| TCGTCACTAACTTAGCCTTTGTGGATTTGTGCTATACATCAAATGCAACCCCGCAGATGTC | |
| GACTAATATCGTATCTGAGAAGACCATTTCCTTTGCTGGTTGCTTTACACAGTGCTACATTT | |
| TCATTGCCCTTCTACTCACTGAGTTTTACATGCTGGCAGCAATGGCCTATGACCGCTATGT | |
| GGCCATATATGACCCTCTGCGCTACAGTGTGAAAACGTCCAGGAGAGTTTGCATCTGCTTG | |
| GCCACATTTCCCTATGTCTATGGCTTCTCAGATGGACTGTTCCAGGCCATCCTGACCTTCCG | |
| CCTGACCTTCTGTAGATCCAATGTCATCAACCACTTCTACTGTGCTGACCCGCCGCTCATTA | |
| AGCTTTCTTGTTCTGATACTTATGTCAAAGAGCATGCCATGTTCATATCTGCTGGCTTCAAC | |
| CTCTCGAGCTCCCTCACCATCGTCTTGGTGTCCTATGGCTTCATTCTTGCTGCGATCCTCCG | |
| GATCAAATCAGCAGAGGGAAGGCACAAGGCATTCTCCACCTGTGGTTCCCATATGATGGC | |
| TGTCACCCTGTTTTATGGGACTCTCTTTTGCATGTATATAAGACCACCAACAGATAAGACT | |
| GTTGAGGAATCTAAAATAATAGCTGTCTTTTACACCTTTGTGAGTCCGGTACTTAATCCAT | |
| TGATCTACAGTCTGAGGAATAAAGATGTGAAGCAGGCCTTGAAGAATGTCCTGAGATGA | |
| AOLFR19 sequences: | |
| METKNYSSSTSGFILLGLSSNPKLQKPLFAIFLIMYLLTAVGNVLIILAIYSDPRLHTPMTTFLSNL | (SEQ ID NO:37) |
| SFMDICFTTVIVPKMLVNFLSETKIISYVGCLIQMTTFMAFGNTDSYLLASMAIDRLVMCNPLH | |
| YDVVMKPWHCLLMLLGSCSISHLHSLFRVLLMSRLSFCASHIIKHFFCDTQPVLKLSCSDTSSSQ | |
| MVVMTETLAVIVTPFLCTIFSYLQIIVTVLRIPSAAGKWKAFSTCGSHLTVVVLFYGSVIYVYFR | |
| PLSMYSVMKGRVATVMYTVVTPMLNPFIYSLRNKDMKRGLKKIRIIRIYS | |
| ATGGAGACAAAGAATTATAGCAGCAGCACCTCAGGCTTCATCCTCCTGGGCCTCTCTTCCA | (SEQ ID NO:38) |
| ACCCTAAGCTGCAGAAACCTCTCTTTGCCATCTTCCTCATCATGTACCTACTCACTGCGGTG | |
| GGGAATGTGCTCATCATCCTGGCCATCTACTCTGACCCGAGGCTCCACACCCCTATGTACT | |
| TTTTTCTCAGCAACTTGTCTTTCATGGATATCTGCTTCACAACAGTCATAGTGCCTAAGATG | |
| CTGGTGAATTTTCTATCAGAGACAAAGATTATCTCTTATGTGGGCTGCCTGATCCAGATGT | |
| ACTTCTTCATGGCATTTGGGAACACTGACAGCTACCTGCTGGCCTCTATGGCCATCGACCG | |
| GCTGGTGGCCATCTGCAACCCCTTACACTATGATGTGGTTATGAAACCATGGCATTGCCTA | |
| CTCATGCTATTGGGTTCTTGCAGCATCTCCCACCTACATTCCCTGTTCCGCGTGCTACTTAT | |
| GTCTCGCTTGTCTTTCTGTGCCTCTCACATCATTAAGCACTTTTTCTGTGACACCCAGCCTG | |
| TGCTAAAGCTCTCCTGCTCTGACACATCCTCCAGCCAGATGGTGGTGATGACTGAGACGTT | |
| AGCTGTCATTGTGACCCCCTTCCTGTGTACCATCTFTCTCCTACCTGCAAATCATCGTCACTG | |
| TGCTCAGAATCCCCTCTGCAGCCGGGAAGTGGAAGGCCTTCTCTACCTGTGGCTCCCACCT | |
| CACTGTAGTGGTCCTGTTCTATGGGAGTGTCATCTATGTCTATTTTAGGCCTCTGTCCATGT | |
| ACTCAGTGATGAAGGGCCGGGTAGCCACAGTTATGTACACAGTAGTGACACCCATGCTGA | |
| ACCCTTTCATCTACAGCCTGAGGAACAAAGATATGAAAAGGGGTTTGAAGAAATTAAGAC | |
| ACAGAATTTACTCATAG | |
| AOLFR20 sequences: | |
| MVEEMITMKNEFILTGFTDHPELKTLLFVXTFAIYLITVVGNISLVALIFTHCRLHTPMYIFLGN | (SEQ ID NO:39) |
| LALVDSCCACAITPKMLENFFSEGKRISLYECAVQFTTLCTVETADCFLLAAVAYDRYVAICNP | |
| LQYHIMMSKKLCIQMTTGAAGNLHSMIHVGLVFLVFCGLNHINHFYCDTLPLYRLSCVDPF | |
| INELVLFIFSGSVQVFTIGSVLISYLYILLTIFRMKSKEGRAKAFSTCASHFSSVSLFYGSIFFLYIRP | |
| NLLEEGGNDWAAILFTIVVPLLNPFIYSLRNKEVISVLRTKILLKIKSQGSVNK | |
| ATGGTTGAAGAAAATCATACCATGAAAAATGAGTTTATCCTCACAGGATTTACAGATCACC | (SEQ ID NO:40) |
| CTGAGCTGAAGACTCTGCTGTTTGTGGTGTTCTTTGCCATCTATCTGATCACCGTGGTGGG | |
| GAATATTAGTTTGGTGGCACTGATATTTACACACTGTCGGCTTCACACACGAATGTACATC | |
| TTTCTGGGAAATCTGGCTCTTGTGGATTCTTGCTGTGCCTGTGCTATTACCCCCAAAATGTT | |
| AGAGAACTTCTTTTCTGAGGGCAAAAGGATTTCCGTCTATGAATGTGCAGTACAGTTTTAT | |
| TTTCTTTGCACTGTGGAAACTGCAGACTGCTTTCTTCTGGCAGCAGTGGCCTATGACCGCT | |
| ATGTGGCCATCTGCAACCCACTGCAGTACCACATCATGATGTCCAAGAAACTCTGCATTCA | |
| GATGACCACAGGCGCCTTCATAGCTGGAAATCTGCATTCCATQATTCATGTAGGGCTTGTA | |
| TTTAGGTTAGTTTTCTGTGGATTGAATCACATCAACCACTTTTACTGTGATACTCTTCCCTT | |
| GTATAGACTCTCCTGTGTTGACCCTTTCATCAATGAACTGGTTCTATTCATCTTCTCAGGTT | |
| CAGTTCAAGTCTTTACCATAGGTAGTGTCTTAATATCTTATCTCTATATTCTTGTTACTATT | |
| TTCAGAATGAAATCCAAGGAGGGAAGGGCCAAAGCCTTTTCTACTTGTGCATCCCACTTTT | |
| CATCAGTTTCATTATTCTATGGATCTATTTTTTTCCTATACATTAGACCAAATTTGCTTGAA | |
| GAAGGAGGTAATGATATACCAGCTGCTATTTTATTTACAATAGTAGTTCCCTTACTAAATC | |
| CTTTCATTTATAGTCTGAGAAACAAGGAAGTAATAAGTGTCTTAAGAAAAATTCTGCTGAA | |
| AATAAAATCTCAAGGAAGTGTGAACAAATGA | |
| AOLFR21 sequences: | |
| MEPRKNVTDFVLLGFTQNPKEQKVLFVMFLLFYILTMVGNLLIVVTVTVSETLGSPMSFFLAGL | (SEQ ID NO:41) |
| TFIDIIYSSSISPRLISDLFFGNNSISFQSFMAQLFIEHLFGGSEVFLLLVMAYDRYVAICKPLHYLV | |
| IMRQWVCVLLLVVSWVGGFLQSVFQLSIIYGLPFCGPNVIDHFFCDMYPLLKLACTDTHVIGLL | |
| VVANGGLSCTIAFLLLLISYGVILHSLKLKLSQKGRQKAIISTCSSHITVVVFFFVPCWMCARiPAR | |
| TFSIDKSVSVFYTVITPMLNPLIYTLRNSEMTSAMKKL | |
| ATGGAGCCAAGGAAAAATGTGACTGACTTTGTCCTCTTGGGCTTCACACAGAATCCAAAG | (SEQ ID NO:42) |
| GAGCAGAAAGTACTTTTTGTTATGTTCTTGCTCTTCTACATTTTGACCATGGTGGGCAACCT | |
| GCTCATTGTAGTGACCGTAACTGTCAGTGAGACCCTGGGCTCACCAATGTCCTTCTTTCTT | |
| GCTGGCTTAACATTTATAGATATCATTTATTCTTCATCCATTTCCCCCAGATTGATTTCAGA | |
| CTTGTTCTTTGGGAATAATTCCATATCCTTCCAATCTTTCATGGCCCAGCTCTTTATCGAGC | |
| ACCTTTTTGGTGGGTCAGAGGTCTTTCTCCTGTTGGTGATGGCCTATGACCGCTATGTGGC | |
| CATCTGTAAGCCCTTGCATTATTTGGTTATCATGAGACAATGGGTGTGTGTTTTGCTGCTG | |
| GTAGTGTCCTGGGTTGGAGGATTTCTGCAATCAGTATTTCAACTTAGCATTATTTATGGGC | |
| TCCCATTCTGTGGCCCCAATGTCATTGATCATTTTTTCTGTGACATGTATCCCTTATTGAAA | |
| CTGGCCTGCACTGACACCCATGYTATTGGCCTCTTAGTGGTGGCCAATGGAGGACTGTCTT | |
| GCACTATTGCGTTTCTGCTCTTACTCATCTCTTATGGTGTCATCCTGCACTCTCTAAAGAAA | |
| CTTAGTCAGAAAGGGAGGCAAAAAGCCCACTCAACCTGCAGTTCCCACATCACTGTGGTTG | |
| TCTTCTTCTTTGTTCCTTGTATTTTTATGTGTGCTAGACCTGCTAGGACCTTCTCCATTGAC | |
| AATCAGTGAGTGTGTTTTATACAGTCATAACCCCAATGGTGTCCCCTTTTCTACACTC | |
| TGAGAAATTCTGAGATGACAAGTGCTATGAAGAAGCTTTAG | |
| AOLFR22 sequences: | |
| MRXXNNXTEFVLLGFSQDPGVXKALFVMFLLTYXXTVVGNLLIVVDIIASPXLGSPMTFLAC | (SEQ ID NO:43) |
| LSFIDAAYSTTISPKLIVGLFCDKKTISFQGCMGQLFIDHFFGGAETLLCDRTAICTL | |
| HYLTIMNRQVCFLLLVXIIGGFVHSAFQIVVYSLPFCGPXVTVTFSCDMHPLLELACTDTYFI | |
| GLTVVVNSGAICMVIFNLLLISYGVILSSLKTYSQEKRGTLSTCSSGSTVLFFTCIFIYVRP | |
| VSNFPTDKFMTVFYTIITHMLSPLIYTLRNSEMRNAIEKLLGTIFIIGGVSVLM | |
| ATGAGACANNNNAACAATATNACAGAATTTGTCCTCCTGGGCTTTTCTCAGGATCCTGGTG | (SEQ ID NO:44) |
| TGNNNAAGCATTATTTGTCATGTTTTTACTCACATACTTACAGTGGTGGGGTCCT | |
| GCTCATTGTNGTGGATATTATTGCCAGCCCTTNNTTGGGTCCCCTTGTATTTCTCCTTG | |
| CCTGCCTGTCATTTATAGATGCTGCATATTCCACTACCATTTCTCCCITGTTTTTGTAGGC | |
| TTATTCTGTGATAAAAAGACTATTTCCTTCCAAGGTTGCATGGGCCAGCTATTATAGACC | |
| ATTTCTTTGGTGGGGCTGAGGTCTTCCTTCTGGTGGTGATGGCCTGTGATCGCTATGTGGC | |
| CATCTGTAAGCCACTGCACTATTTGACCATCATGTTCGACAGGTTTGCTTCCTTCTGTTGG | |
| TNNTNNCCATGATTGGAGGTTTTGTACATTCTGCGTTTCAAATTGTTGTGTACAGTCTCCCT | |
| TTCTGTGGTCCCNATGTCATTGTTCATTTCAGTTGTGACATGCACCCATTACTGGTCTGGC | |
| ATGCACTGACACCTACTTTATAGGCCTCACTGTTGTTGTCTTAGTGGAGCTTCTGTATG | |
| GTCATTTTCAACCTTCTGTTAATCTCCTATGGAGTCATCCTTGCTCCCTTTTCTTACAG | |
| TCAGGAAAAGAGGGGTAAAGCCTTGTCTACCTGCAGCTCCGGCAGTACCGTTGTTGTCCTC | |
| TTTTTTGTACCCTGTATTTTCATATATGTTAGACCTGTTTCAAACTTTCCTACTGATTGTT | |
| CATGACTGTGTTTTATACCATTATCACACACATGCTGAGTCCTTTTTATATACGTTGAGA | |
| AATTCAGAGATGAGAAATGCTATAGAAAAACTCTTGGGTGTTTCTATATTATA | |
| TAGGAGGAGTGTCCGTCCTCATGTAG | |
| AOLFR23 sequences: | |
| MAKNLTRVTEFILMGFMDHPKLEIPLFLVFLSFYLVTLLGNVGMIMLIQVTYTPMYFFLS | (SEQ ID NO:45) |
| HLSLLDACYTSVITPQILATLATGKTVISYGHCAAQFFLFTICAGTECFLLAVMAYDRYAAIRNP | |
| LLYTVAMNPRTCWSLVVGAYVCGVSGAILRTTCTFTLSFCKDNQTTFFCDLPPLLTACSDTA | |
| NIEIVIIFFGNFVILANASVILISYLLIIKTILKVKSSGGRAKTFSTGASHITAVTFFGALIFMYLQS | |
| GSGKSLEEDKVVSVEYTVVIPMLNPLIYSLPTNKDVKDAERKVARITQVSLSM | |
| ATGGCCAAGAATAATCTCACCAGAGTAAGCGAATTCATTCTCATGGGCTTTATGGACCACC | (SEQ ID NO:46) |
| CCAAATTGGAGATTCCCCTCTTTCTGGTGTTTCTGAGTTCTACCTAGTCACCCTTCTTGGG | |
| AATGTGGGGATGATTATGTTAATCCAAGTAGATGTCAAACTCTACACCCCTTGTACTTCT | |
| TCCTGAGCCACCTCTCCCTGCTGGATGCCTGTTACACCTCAGTCATCACCCCTCAGATCCTA | |
| GCCACATTGGCCACAGGCAAAACGGTCATCTCCTACGGCCACTGTGCTGCCCAGTTCTTT | |
| TATTCACCATCTGTGCAGGCACAGAGTGCTTTCTGCTGGCAGTGATGGCCTATGATCGCTA | |
| TGCTGCCATTCGCAACCCACTGCTCTATACCGTGGCCATGAATCCCAGGCTCTGCTGGAGC | |
| CTGGTGGTAGGAGCCTATGTCTGTGGGGTGTCAGGAGCCATCCTGCGTACCACTGCACCT | |
| TCACCCTCTCCTTCTGTAAGGACAATCAAATAAACTTCTTGTTCTGTGACCTCCCACCCCTG | |
| CTGAAGCTTGCCTGCAGTGACACAGCAAACATCGAGATTGTCATGATCTTCTTTGGCTTT | |
| TTGTGATTTTGGCCAATGCCTCCGTCATCCTGATTTCCTATCTGCTCATCATCTGACCATT | |
| TTGAAAGTGAAGTCTTCAGGTGGCAGGGCCAAGACTTTCTCCACATGTGCCTCTCACATCA | |
| CTGCTGTGGCCCTTTTCTTTGGAGCCCTTATCTTCATGTATCTGCAAAGTGGCTCAGGCATT | |
| TCTCTGGAGGAAGACAAAGTCGTGTCTGTCTTCTATACAGTGGTCATCCCCATGCTGTCC | |
| CTCTGATCTACAGCTTAAGAAACAAAGATGTAAAAGACGCCTCAGTAGGTCGCTAGGA | |
| GACTCCAGGTGTCCCTGAGCATGTAG | |
| AOLFR25 sequences: | |
| METGNLTWVSDFVFLGLSQTRRELQRFLFLMFLFVYITTVMGNILIIITVTSDSQLHTPMYFLLRN | (SEQ ID NO:47) |
| LAVLDLCFSSVTAPKMLVDLLSEKKTISYQGCMGQWFFHFLGGAMVFFLSVMAFDRLIAISRPL | |
| RTTVTTQLWVGLVVATWVGGFVHSIVQLALMLPLPFCGPMLDNFYCDVPQVLTACTDT | |
| SLLEFLKISNSGLLDVVWFFLLLMSYLFILVMLRSHPGEARRKAASTCTTHIIVVSMIFVPSIYLY | |
| ARPFTPFPMDKLVSIGHTVMTPMLNPMIYTLRNQDMQAAVRRLGRHRLV | |
| ATGGAAACAGGGAACCTCACGTGGGTATCAGACTTTGTCTTTCCTGGGGCTCTCGCAGACTC | (SEQ ID NO:48) |
| GGGAGCTCGAGCGTTTCCTGTTTCTAATGTTCCTGTTGTCTACATCACCACTGTTATGGGA | |
| AACATCCTTATCATCATCACAGTGACCTCTGATTCCCAGCTCCACACACCCATGTACTCT | |
| GCTCGGAAACCTGGCTGTCCTAGACCTCTGTTTCTCTTCAGTCACTGCTCCCAATTTGCTAG | |
| TGGACCTCCTCTCTGAGAAGAAAACCATCTCTTACCAGGGCTGCATGGGTCAGATCTCT | |
| CTTCCACTTTTTGGGAGGTGCCATGGTCTTCTTCCTCTCAGTGATGGCCTTTGACCGCCTCA | |
| TTGCCATCTCCCGGCCCCTCCGCTATGTCACCGTCATGAACACTCAGCTCTGGGTGGGGCT | |
| GGTGGTAGCCACCTGGGTGGGAGGCTTGTCCACTCTATGTCCAGTGGCTCTGATGCTC | |
| CCACTGCCCTTCTGTGGCCCCAACATTITGGATAACTTCTACTGTGATGTTCCCCTGTACT | |
| GAGACTTGCCTGCACTGACACCTCACTGCTGGAGTTCCTCAAGATCTCCTCAGTGGGCTG | |
| CTGGATGTCGTCTGGTTCTTCCTCCTCCTGATGTCCTACTTATTCATCCTGGTGATGCTGAG | |
| GTCACATCCAGGGGAGGCAAGAAGGAAGGCAGCTTCCACCTGCACCACCCACATCATCGT | |
| GGTTTCCATGATCTTCGTTCCAAGCATTTACCTCTATGCCCGGCCCTCACTCCATTCCCTA | |
| TGGACAAGCTTGTGTCCATCGGCCACACAGTCATGACCCCCATGCTCAACCCCATGATCTA | |
| TACCCTGAGGAACCAGGACATGCAGGCAGCAGTGAGAAGATTAGGGAGACACCGGCTGGT | |
| TTGA | |
| AOLFR26 sequences: | |
| MAAKNSSVTEFILEGLTHQPGLRIPLFFLFLGFYTVTVVGNLGLITLIGLNSHLHTPMTTFLFNLS | (SEQ ID NO:49) |
| LIDFGFSTTITPKMLMSFVSRKNIISFTGCMTQLFFFCFFVVSESFILSAMAYDRYVAICNPLLYT | |
| VTMSCQVCLLLLLGAYGMGFAGAMAIITGSIMNLTFCADNLVNHFMCDILPLLELSCNSSYMN | |
| ELVVFIVVAVDVGMPIVTFISYALILSSILHNSSTEGRSKAFSTCSSHIITSLFFGSGAFMYLKP | |
| LSILPLEQGKVSSLFYTIIVPVLNPLIYSLRNKDVKVALRRTLGRKIFS | |
| ATGGCAGCCAAAAACTCTTCTGTGACAGAGTTTATCCTCGAAGGCTTAACCCACCAGCCGG | (SEQ ID NO:50) |
| GACTGCGGATCCCCCTCTTCTTCCTGTTTCTGGGTTTCTACACGGTCACCGTGGTGGGGT | |
| CCTGGGCTTGATAACCCTGATTGGGCTGAACTCTCACCTGCACACTCCCATGTACTTCTTCC | |
| TTTTTAACCTCTCTTTTAATAGATTTCTGTTTCTCCACTACCATCACTCCCAAAATGCTGATG | |
| AGTTTTGTCTCAAGGAAGAACATCATTTCCTTCACAGGGTGATGACTCAGCTCTTCTTCTT | |
| CTGCTTCTTTGTCGTCTCTGAGTCCTTCATCCTGTCAGCGATGGCGTATGACCGCTACGTGG | |
| CCATCTGTAACCCACTGTGTACACAGTCACCATGTCTTGCCAGGTGTGTTGCTCCTTG | |
| TTGGGTGCCTATGGGATGGGGTTTGCTGGGGCCATGGCCCACACAGGAAGCATAATGTC | |
| CTGACCTTCTGTGCTGACAACCTTGTCAATCATTTCATGTGTGACATCCTCCTCTCCTTGA | |
| GCTCTCCTGCAACAGCTCTTACATGAATGAGCTGGTGGTCTTTATTGTGGTGGCTGTTGAC | |
| GTTGGAATGCCCATGTCACTGTCTTTATTTCTTATGCCCTCATCCTCTCCAGCATTCTACA | |
| CAACAGTTCTACAGAAGGCAGGTCCAAAGCCTTTAGTACTTGCAGTTCCCACATAATTGTA | |
| GTTTCTGTTTTCTTTGGTTCTGGTGCTTTCATGTATCTCAAACGCCTTTCCATCCTGCCCCTC | |
| GAGCAAGGGAAAGTGTCCTCCCTGTTCTATACCATAATAGTCCCCGTGTTAAACCCATTAA | |
| TCTATAGCTTGAGGAACAAGGATGTCAAAGTTGCCCTGAGGAGAACTTTGGGCAGAAA | |
| TCTTTTCTTAA | |
| AOLFR27 sequences: | |
| MPSQNYSIISEFNLFGFSAFPQHLLPILFLLYLLMFLFTLLGNLLIMATIWIEHRLHTPMYLFLCTL | (SEQ ID NO:51) |
| SVSEILFTVAITPRMLADLLSTHHSITFVACANQMFFSFMFGFTHSFLLLVMGYDRYVAICHPLR | |
| YNVLMSPRDCAHLVACTWAGGSVMGMMVTIVFHLTFCGSNVHTHTTCHVLSLLKLACENKT | |
| SSVIMGVMLVCVTALIGCLFLIILSYVFIVAAILRIPSAEGRHKTFSTCVSHLTVVVTHYSFASFIY | |
| LKPKGLHSMYSDALMATTYTVFTPFLSPIIFSLRNKELKNAThKNFYRXFCPPSS | |
| ATGCCTAGTCAGAACTATAGCATCATATCTGAATTTAACCTCTITGGCTTCTCAGCCTTCCC | (SEQ ID NO:52) |
| CCAGCACCTCCTGCGCATCTTGTTCCTGCTGTACCTCCTGATGTTCCTGTTCACATTGCTGG | |
| GCAACCTTCTCATCATGGCCACAATCTGGATTGAACACAGACTGCACACACCCATGTACGT | |
| CTTCTTGTGCACCCTCTCCGTCTCTGAGATTCTGTTCACTGTTGCCATCAGCCCTCGGATGC | |
| TGGCTGATCTGCTTITCCACCCATCKFfCCATCACCTTTGTGGCTTGTGCCAACCAGATGTTC | |
| TTCTCCTTCATGTTTGGCTTCACTCACTCCTTCCTTCTCCTGGTCATGGGCTATGATCGCTA | |
| TGTGGCCATCTGCCACCCACTGCGTTACAATGTGCTCATGAGCCCCCGTGACTGTGCCCAT | |
| CTTGTGGCCTGTACCTGGGCTGGTGGCTCAGTCATGGGGATGATGGTGACAACGATAGTTT | |
| TCCACCTCACTTTTCTGTGGGTCTAATGTGATCCACCATTTTTTCTGTCATGTGCTTTCCCTCT | |
| TGAAGTTGGCCTGTGAAAACAAGACATCATCTGTCATCATGGGTGTGATGCTGGTGTGTGT | |
| CACAGCCCTGATAGGCTGTTTATTCCTCATCATCCTCTCCTATGTCTTCATTGTGGCTGCCA | |
| TCTTGAGGATTCCCTCTGCCGAAGGCCGGCACAAGACATTTTCTACGTGTGTATCCCACCT | |
| CACTGTGGTGGTCACGCACTATAGTTTTGCCTCCTTTATCTACCTCAAGCCCAAGGGCCTCC | |
| ATTCTATGTACAGTGACGCCTTGATGGCCACCACCTATACTGTCTTCACCCCCTTCCTTAGC | |
| CCAATCATTTTCAGCCTAAGGAAGAAGGAGCTGAAGAATGCCATAAATAAAAACTTTTACA | |
| GAAAATTCTGTCCTCCAAGTTCCTGA | |
| AOLFR28 sequences: | |
| MPNFTDVTEFTLLGLTCRQELQVLFFVVFLAVYMITLLGNIGMIILISISPQLQSPMTTFLSHLSF | (SEQ ID NO:53) |
| ADVCFSSNVTPKMLENLLSETKTISYVGCLVQCTTFLAVVHVEVYILAVMAFDRYMAGCXPLL | |
| YGSKMSRTVCVRLISVXYXYGFSVSLICTLWTYGLYECGNFEINHFYCADPPLIQIACGRVHIKE | |
| ITMIVIAGINFTYSLSVVLISYTLIVVAVLRMRSADGRRKAFSTCGSHLTAVSMFYGTPLFMYLR | |
| RPTEESVEQGKMVAVFYTTVIPMLNPMIYSLRMTDVKEAVNKAITKTYVRQ | |
| ATGCCTAATTTCACGGATGTGACAGAATTTACTCTCCTGGGGCTGACCTGTCGTCAGGAGG | (SEQ ID NO:54) |
| TACAGGTTCTCTTTTTTGTGGTGTTCCTAGCGGTTTACATGATCACTCTGTTGGGAAATATT | |
| GGTATGATCATTTTGATTAGCATCAGTCCTCAGCTTCAGAGTCCCATGTACTTTTTCCTGAG | |
| TCATCTGTCTTTTGCGGACGTGTGCTTCTCCTCCAACGTTACCCCCAAAATGCTGGAAAACT | |
| TATTATCAGAGACAAAAACCATTTCCTATGTGGGATGCTTGGTGCAGTGCTACTTTTTCAT | |
| TGCGGTTGTCCACGTGGAGGTCTATATCGTGGCTGTGATGGCGTTTGACAGGTACATGGGC | |
| GGCTGCAANCCTCTGCTTTATGGCAGTAAAATGTCTAGGACTGTGTGTGTTGGCTCATCT | |
| CTGTGNNNTATGNNTATGGATTCTCTGTCAGGGTAATATGCACACTATGGACTTATGGCTT | |
| ATACTTCTGTGGAAACTTTGAAATCAATCACTTCTATTGTGCAGATCCCCCTCTCATCCAGA | |
| TTGCCTGTGGGAGAGTGCACATCAAAGAAATCACAATGATTGTTATTGCTGGATTAACTT | |
| CACATATTCCCTCTCGGTGGTCCTCATCTCCTACACTCTCATTGTAGTAGCTGTGCTACGCA | |
| TGCGCTCTGCCGATGGCAGGAGGAAGGCGTTGTCCACCTGTGGGTCCCACTTGACGGCTGT | |
| TTCTATGTTTTATGGGACCCCCATCTTCATGTATCTCAGGAGACCCACTGAGGAATCCGTA | |
| GAGCAGGGCAAATGGTGGCTGTGTTTTACACCACAGTAATTCCTATGTTGAATCCCATGA | |
| TCTACAGTCTGAGAAATAAGGATGTAAAAGAAGCAGTCAACAAAGCAATGACCAAGACAT | |
| ATGTGAGGCAGTAA | |
| AOLFR29 sequences: | |
| MMSFAPNASHSPVFLLLGFSRANISYTLLFFLFLMYLTITILGNVTLVLLISWDSRLHSPMYYLLR | (SEQ ID NO:55) |
| GLSVIDMGLSTVTLPQLLAHLVSHYPTIPAARCLAQFFFFYAFGVTDTLVIAVMALDRYVAICD | |
| PLHYALVMNHQRCACLLALSWVVSILHTMLRVGLVLPLCWTGDAGGNVNLPHFFCDHRPLLR | |
| ASCSDIHSNELAIFFEGGFLMLGPCALIVLSYVRIGAAILRLPSAAGRRRAVSTCGSHLTMVGFL | |
| YGTIICVTTQPPFQNSQYQDMVASVMYTAITPLANPFVYSLHNKDVKGALCRLLEWVKVDP | |
| ATGATGAGCYTTGCCCCTAATGCTTCACACTCTCCGGTTTTTTTGCTCCTTGGGTTCTCGAG | (SEQ ID NO:56) |
| AGCTAACATCTCCTACACTCTCCTCTTCTTCCTGTTCCTGGCTATTTACCTGACCACCATAC | |
| TGGGGAATGTGACACTGGTGCTGCTCATCTCCTGGGACTCCAGACTGCACTCACCCATGTA | |
| TTATCTGCTTCGTGGGCTCTCTGTGATAGACATGGGGCTATCCACAGTTACACTGCCCCAG | |
| TTGCTGGCCCATTTGGTCTCTCATTACGCAACCATTCCTGCTGCCCGCTGGTTGGCTCAGTT | |
| CTTTTTCTTCTATGCATTTGGGGTTACAGATACACTTGTCATTGCTGTCATGGCTCTGGATC | |
| GCTATGTGGCCATCTGTGACCCCCTGCACTATGCTTTGGTAATGAATCACCAACGGTGTGC | |
| CTGCTTACTAGCCTTGAGCTGGGTGGTGTCCATACTGCACACCATGTTGCGTGTGGGACTC | |
| GTCCTGCCTCTTTGCTGGACTGGGGATGGTGGGGGCAACGTTAACCTTCCTCACTTCTTTTG | |
| TGACCACGGGCCACTTCTGGGAGCCTCTTGTTCTGACATACATTGTAATGAGCTGGGGATA | |
| TTCTTTGAGGGTGGGTCCTTATGCTGGGCCCCTGTGCCCTCATTGTACTCTCTTATGTCCG | |
| AATTGGGGCCGCTATTCTACGTTTGCCTTCAGCTGCTGGTCGCCGCCGAGCAGTCTCCACC | |
| TGTGGATCCCACCTCACCATGGTTGGTTTCCTCTACGGCACCATCATTTGTGTCTACTTCCA | |
| GCCTCCCTTCCAGAACTCTCAGTATCAGGACATGGTGGCTTCAGTAATGTATACTGCCATT | |
| ACACCTTTGGCCAACCCATTTGTGTATAGCCTCCACAATAAGGATGTCAAGGGTGCACTCT | |
| GCAGGCTGCTTGAATGGGTGAAGGTAGACCCCTGA | |
| AOLFR30 sequences: | |
| MGFLSPMHPCRPPTQRRMAAGNIHSTVTEFILKGLTKRADLQLPLFLLFLGIYLVTIVGNLGMIT | (SEQ ID NO:57) |
| LICLNSQLHTPMYTTLSNLSLMDLCYSSVITPKMLTNFVSEKNIISYAGCMSQLTTFLVFVIAEC | |
| YMLTVMAYDRYVXXCHPLLYNIIMSHHTCLLLVAVVYAIGLIGSTIETGLMLKLPYCEHLISHY | |
| FCDILPLMiKLSCSSTYDVEMTVFFSAGFNIIVTSLTVLVSYTFILSSILGISTTEGRSKAFSTCSSHL | |
| AAVGMFYGSTAFMYLKPSTISSLTQENVASVFYTTVIPMLNPLIYSLRNKEVKAAVQKTLRGK | |
| LF | |
| ATGGGGTTCTTGTCTCCCATGCATCCCTGCAGGCCTCCCACCCAGAGGAGAATGGCTGCAG | (SEQ ID NO:58) |
| GAAATCACTCTACAGTGACAGAGTTCATTCTCAAGGGTTTAACGAAGAGAGCAGACCTCC | |
| AGCTCCCCCTCTTTCTCCTCTTCCTCGGGATCTACTTGGTCACCATCGTGGGGAACCTGGGC | |
| ATGATCACTCTAATTTGTCTGAACTCTCAGCTGCACACCCCCATGTACTACTTTCTCAGCAA | |
| TCTGTCACTCATGGATCTCTGCTACTCCTCCGTCATTACCCCTAAGATGGTGGTGAACTTTG | |
| TGTCAGAGAAAAACATCATCTCCTACGCAGGGTGCATGTCACAGCTCTACTTCTTCCTTGT | |
| TTTTGTCATTGCTGAGTGTTACATGCTGACAGTGATGGCGTACGACCGCTATGTTGNCNTC | |
| TGCCACCCTTTGCTTTACAACATCATTATGTCTCATCACACCTGCCTGCTGCTGGTGGCTGT | |
| GGTCTACGCCATCGGACTCATTGGCTCCACAATAGAAACTGGCCTCATGTTAAAACTGCCC | |
| TATTGTGAGCACCTCATCAGTCACTACTTCTGTGACATCCTCCCTCTCATGAAGCTGTCCTG | |
| CTCTAGCACCTATGATGTTGAGATGACAGTCTTCTTTTGGGCTGGATTCAACATCATAGTC | |
| ACGAGCTTAACAGTTCTTGTTTCTTACACCTTCATTCTCTCCAGCATCCTCGGCATCAGCAC | |
| CACAGAGGGGAGATCCAAAGCCTTCAGCACCTGCAGCTCCCACCTTGCAGCCGTGGGAAT | |
| GTTCTATGGATCAACTGCATTCATGTACTTAAAACCCTCCACAATCAGTTCCTTGACCCAG | |
| GAGAATGTGGCGTCTGTGTTCTAGACCACGGTAATCCCCATGTTGAATCCCCTAATCTACA | |
| GCCTGAGGAACAAGGAAGTAAAGGCTGCCGTGCAGAAAACGCTGAGGGGTAAACTGTTTT | |
| GA | |
| AOLFR31 sequences: | |
| MGTGNDTTVVEFTLLGLSEDTTVCAILFLVFLGIYVVTLMGNISIIVLIRTTSHHLHTPMYIFLCHL | (SEQ ID NO:59) |
| AFVDIGYSSSVTPVMLMSFLRKETSLPVAGCVAQLCSVVTFGTAECFLLAAMAYDRYVAlCSP | |
| LLYSTCMSPGVCIILVGMSYLGGCVNAWTFIGCLLRLSFCGPNKVNHFFCDYSPLLKLACSHDF | |
| TFEIIPAISSGSHVATVCVIAISYIYILITILKMHSTKGPIIjKAFSTCTSHLTAVTLFYGTITFIYVMP | |
| KSSYSTDQNKVVSVFYTVVIPMLNPLIYSLRNKEIKGALKRELRIKIFS | |
| ATGGGGACTGGAAATGACACCACTGTGGTAGAGTTTACTCTTTTGGGGTTATCTGAGGATA | (SEQ ID NO:60) |
| CTACAGTTTGTGCTATTTTATTTCTTGTGTTTCTAGGAATTTATGTTGTCACCTTAATGGGT | |
| AATATCAGCATAATTGTATTGATCAGAAGAAGTCATCATCTTCATACACCCATGTACATTT | |
| TCCTCTGCCATTTGGCCTTTGTAGACATTGGGTACTCCTCATCAGTCACACCTGTCATGCTC | |
| ATGAGCTTCCTAAGGAAAGAAACCTCTCTCCCTGTTGCTGGTTGTGTGGCCCAGCTCTGTT | |
| CTGTAGTGACGTTTGGTACGGCCGAGTGCTTCCTGCTGGCTGCCATGGCCTATGATCGCTA | |
| TGTGGCCATGTGCTCACCCCTGCTCTACTCTACCTGCATGTCCCCTGGAGTCTGCATCATCT | |
| TAGTGGGCATGTCCTACCTGGGTGGATGTGTGAATGCTTGGACATTCATTGGCTGCTTATT | |
| AAGACTGTCCTTCTGTGGGCCAAATAAAGTCAATCACTTTTTCTGTGACTATTCACCACTTT | |
| TGAAGCTTGCTTGTTGCCATGATTTTACTTTTGAAATAATTCCAGCTATCTCTTCTGGATCT | |
| ATCATTGTGGCCACTGTGTGTGTCATAGCCATATCCTACATCTATATCCTCATGACCATCCT | |
| GAAGATGCACTCCACCAAGGGCCGCCACAAGGCCTTCTCCACCTGCACCTCCCACCTCACT | |
| GCAGTCACTCTGTTCTATGGGACCATTACCTTCATTTATGTGATGCCCAAGTCCAGCTACTC | |
| AACTGACCAGAACAAGGTGGTGTCTGTGTTCTACACCGTGGTGATTCCCATGTTGAACCCC | |
| CTGATCTACAGCCTCAGGAACAAGGAGATTAAGGGGGCTCTGAAGAGAGAGCTTAGAATA | |
| AAAATATTTTCTTGA | |
| AOLFR32 sequences: | |
| MNSLKDGNIITALTGFILLGLTDDPILRVILFMIILSGNLSIIILIRISSQLHHPMTTFLSHLAFADM | (SEQ ID NO:61) |
| AYSSSVTPNMLVNFLVERNTVSYLGCAIQLGSAAFFATVECVLLAAMAYDRFVAICSPLLYSTK | |
| MSTQVSVQLLLVVYLAGFLIAVSYTTSFTTLLFCGPNQVNHFFCDFAPLLELSCSDISVSTVVLSF | |
| SSGSIIVVTVCVIAVCYIYILITILKMRSTEGHHKAFSTCTSHLTVVTLFYGTITFIYVMPNFSYST | |
| DQNKVVSVLYTVVIPMLNPLIYSLRNKEIKGALKRIELVRKILSHDACTTSRTSNNDIT | |
| ATGAATTCCCTGAAGGACGGGAATCACACCGCTCTGACGGGGTTCATCCTATTGGGCTTAA | (SEQ ID NO:62) |
| CAGATGATCCAATCCTTCGAGTCATCCTCTTCATGATCATCCTATCTGGTAATCTCAGCATA | |
| ATTATTCTTATCAGAATTTCTTCTCAGCTCCATCATCCTATGTATTTCTTTCTGAGCCACTT | |
| GGCTTTTGGTGACATGGCCTATTCATCTTCTGTCACACCCAACATGCTTGTAAACTTCCTGG | |
| TGGAGAGAAATACAGTCTCCTACCTTGGATGTGCCATCCAGCTTGGTTGAGCGGCTTTCTT | |
| TGCAAGAGTCGAATGCGTCCTTCTGGCTGCCATGGCCTATGACCGCTTTGTGGCAATTTGC | |
| AGTCCACTGCTTTATTCAACGAAAATGTCCACAGAAGTCAGTGTCCAGCTACTCTTAGTAG | |
| TTTACATAGCTGGTTTTCTCATTGCTGTCTCCTATACTACTTCCTTCTATTTTTTACTCTTCT | |
| GTGGACCAAATCAAGTCAATCATTTTTTCTGTGATTTCGCTCCCTTACTTGAACTCTCCTGT | |
| TCTGATATCAGTGTCTCCAGAGTTGTTCTCTCATTTTCTTCTGGATCCATCATTGTGGTCAC | |
| TGTGTGTGTCATAGCCGTCTGCTACATCTATATCCTCATCACCATCCTGAAGATGCGCTCCA | |
| CTGAGGGGCACCACAAGGCCTTCTCCACCTGCACTTCCCACCTCACTGTGGTTACCCTGTT | |
| CTATGGGACCATTACCTTCATTTATGTGATGCCCAATTTTAGCTACTCAACTGACCAGAAC | |
| AAGGTGGTGTCTGTGTTGTACACAGTGGTGATTCCCATGTTGAACCCCCTGATCTACAGCC | |
| TCAGGAACAAGGAGATTAAGGGGGCTCTGAAGAGAGAGCTTGTTAGAAAAATACTTTCTC | |
| ATGATGCTTGTFFATTTTAGTAGAACTTCAAATAATGATATTACATAG | |
| AOLFR34 sequences: | |
| MLEGVEHLLLLLLLTDVNSKELQSGNQTSVSHFILVGLHHPPQLGAPLFLAFLVIYLLTVSGNG | (SEQ ID NO:63) |
| LIILTVLVDIRIIIRPMCLFLCHLSFLDMTISCAIVPKMLAGFLLGSRIISFGGCVIQLFSFLIFLGCT | |
| ECFLYTLMAYDRFLAICKPLHYATIMTHRVCNSLALGTWLGGTIHSLFQTSFVFRLPFCGPNRV | |
| DYIFCDIPAMLRLACADTAINELVTFADIGFLALTCFMLILTSYGYIVAAILRIPSADGRRNAFST | |
| CAAHLTVVIVYYVPCTFIYLRPCSQEPLDGVVAVFYTVITPLLNSIIYTLCNKEMKAALQRLGG | |
| HKEVQPH | |
| ATGTTAGAGGGTGTTGAGCATCTCCTTCTGCTACTTCTTTTGACAGATGTGAACAGCAAGG | (SEQ ID NO:64) |
| AACTGCAAAGTGGAAACCAGACTTCTGTGTCTCACTTCATTTTGGTGGGCCTGCACCACCC | |
| ACCACAGCTGGGAGCGCCACTCTTCTTAGCTTTCCTTGTCATCTATCTCCTCACTGTTTCTG | |
| GAAATGGGCTCATCATCCTCACTGTCTTAGTGGACATCCGGCTCCATCGTCCCATGTGCTT | |
| GTTCCTGTGTCACCTCTCCTTCTTGGACATGACCATTTCTTGTGCTATTGTCCCCAAGATGC | |
| TGGCTGGCTTTCTCTTGGGTAGTAGGATTATCTCCTTTGGGGGCTGTGTAATCCAACTATTT | |
| TCTTTCCATTTCCTGGGCTGTACTGAGTGCTTCCTTTACACACTCATGGCTTATGACCGTTT | |
| CCTTGCCATTTGTAAGCCCTTACACTATGCTACCATCATGACCCACAGAGTCTGTAACTCCC | |
| TGGCTTTAGGCACCTGGCTGGGAGGGACTATCCATTCACTTTTCCAAACAAGTTTTGTATT | |
| CCGGCTGCCCTTCTGTGGCCCCAATCGGGTCGACTACATCTTCTGTGACATTCCTGCCATGC | |
| TGCGTCTAGCCTGCGCCGATACGGCCATCAACGAGCTGGTCACCTTTGGAGACATTGGCTT | |
| CCTGGCCCTCACCTGCTTCATGCTCATCCTCACTTCCTATGGCTATATTGTAGCTGCCATCC | |
| TGCGAATTCCGTCAGCAGATGGGCGCCGCAATGCCTTCTCCACTTGTGCTGCCCACCTCAC | |
| TGTTGTCATTGTTTACTATGTGCCCTGCACCTTCATTTACCTGCGGCCTTGTTCACAGGAGC | |
| CCCTGGATGGGGTGGTAGCTGTCTTTTACACTGTCATCACTCCCTTGCTTAACTCCATCATC | |
| TACACACTGTGCAACAAAGAAATGAAGGCAGCATTACAGAGGCTAGGGGGCCACAAGGAA | |
| GTGCAGCCTCACTGA | |
| AOLFR35 sequences: | |
| MEPLNRTEVSEFFLKGFSGYPALEHLLFPLCSAMYLVTLLGNTAIMAVSVLDIHLHTPVYFFLG | (SEQ ID NO:65) |
| NLSTLDICYTPTFVPLMLVHLLSSRKTISFAVCAIQMCLSLSTGSTECLLLAITAYDRYLAICQPL | |
| RYHVLMSHRLCVLLMGAAWVLCLLKSVTEMVISMRIPFCGIIHVVSHFTCKIILAVLKLACGNT | |
| SVSEDFLLAGSILLLPVPLAFICLSYLLILATILRVPSAARCCKAFSTCLAHLAVVLLFYGTIIFMY | |
| LKPKSKEAIHISDEVFTVLYAMVTTMLNPTIYSLRNKEVKEAARKVWGRSRASR | |
| ATGGAGCCGCTCAACAGAACAGAGGTGTCCGAGTTCTTTCTGAAAGGATTTTCTGGCTACC | (SEQ ID NO:66) |
| CAGCCCTGGAGCATCTGCTCTTCCCTCTGTGCTCAGCCATGTACCTGGTGACCCTCCTGGG | |
| GAACACAGCCATCATGGCGGTGAGCGTGCTAGATATCCACCTGCACACGCCCGTGTACTTC | |
| TTCCTGGGCAACCTCTCTAGCCTGGACATCTGCTACACGCCCACCTTTGTGCCTCTGATGCT | |
| GGTCCACCTCCTGTCATCGCGGAAGACCATCTCCTTTGCTGTCTGTGCCATCCAGATGTGTC | |
| TGAGCCTGTCCACGGGCTCCACGGAGTGCCTGCTACTGGCCATCACGGCCTATGACCGCTA | |
| CCTGGCCATCTGCCAGCCACTCAGGTACCACGTGCTCATGAGCCACCGGCTCTGCGTGCTG | |
| CTGATGGGAGCTGCCTGGGTCCTCTGCCTGCTCAAGTCGGTGACTGAGATGGTCATCTCCA | |
| TGAGGCTGCCCTTCTGTGGCCACCACGTGGTCAGTCACTTCACCTGCAAGATCGTGGCAGT | |
| GCTGAAGCTGGCATGCGGCAACACGTCGGTCAGCGAAGACTTCCTGCTGGCGGGCTCCAT | |
| CCTGCTGCTGCCTGTACCCCTGGCATTCATCTGCCTGTCCTACTTGCTCATCCTGGCCACCA | |
| TCCTGAGGGTGCCCTCGGCCGCCAGGTGCTGCAAAGCCTTCTCCACCTGCTTGGCACACCT | |
| GGCTGTAGTGCTGCTTTTCTACGGCACCATCATCTTCATGTACTTGAAGCCCAAGAGTAAG | |
| GAAGCCCACATCTCTGATGAGGTCTTCAGAGTCCTCTATGCCATGGTCACGACCATGCTGA | |
| ACCCGACCATCTACAGCCTGAGGAACAAGGAGGTGAAGGAGGCCGCCAGGAAGGTGTGGG | |
| GCAGGAGTGGGGCCTCCAGGTGA | |
| AOLFR36 sequences: | |
| MYLVTVLRNLLSILAVSSDSHPHTPMTTFLSNLCWADIGFTLATVPKMIVDMGSHSKVISYGG | (SEQ ID NO:67) |
| CLTQMSFLVLFAGIVDMFLTVMAYDCFVAICRPLHYPVIVNIPHLCVFFVLVSFFLSLLDSQLHS | |
| WIVLQFTFFKNVEISNFVCEPSQLLKLASYDSVLNSIFITTDNTMFGFLPISGILLSYYKIVPSILRIS | |
| SSDGKYKALFSACGCHLAVYCLFYGTGIGVYLTSAVAPPLRNGMVASVMYAVVTPMLNPFIYS | |
| LRNRDIQSALWRVCNKTVESHDLFHPFSCVVEKGQPHSIPTSANPAP | |
| ATGTATCTGGTCACGGTGCTGAGGAACCTGCTCAGCATCCTGGCTGTCAGCTCTGACTCCC | (SEQ ID NO:68) |
| ACCCCCACACACCCATGTACTTCTTCCTCTCCAACCTGTGCTGGGCTGACATCGGTTTCACC | |
| TTGGCCACGGTTCCCAAAATGATTGTGGACATGGGGTCGCATAGCAAAGTCATCTCTTATG | |
| GGGGCTGCCTGACACAGATGTCTTTCTTGGTACTTTTTGCATGTATAGTAGACATGTTCCT | |
| GACTGTGATGGCTTATGACTGCTTTGTAGCCATCTGTCGCCGTCTGCACTACCCAGTCATC | |
| GTGAATCCTCACCTCTGTGTCTTCTTCGTTTTGGTGTCCTTTTTCCTTAGCCTGTTGGATTCC | |
| CAGCTGCACAGTTGGATTGTGTTACAATTCACCTTCTTCAAGAATGTGGAAATCTCTAATT | |
| TTGTCTGTGAGCCATCTCAACTTCTCAAGCTTGCCTCTTATGACAGCGTCATCAATAGCATA | |
| TTCATATATTTTGATAATACTATGTTTGGTTTTCTTCCCATTTCAGGGATCCTTTTGTCTTAC | |
| TATAAAATTGTCCCCTCCATTCTAAGGATTTCATCATCAGATGGGAAGTACAAAGCCTTCT | |
| CAGCCTGTGGCTGTCACCTGGCAGTTGTTTGCTTATTTTATGGAACAGGCATTGGCGTGTA | |
| GCTGACTTCAGCTGTGGCACCACCCCTCAGGAATGGTATGGTGGCGTGAGTGATGTACGCT | |
| GTGGTCACCCCCATGCTGAACCCTTTCATCTACAGCCTGAGAAACAGGGACATTCAAAGTG | |
| CCCTGTGGAGGGTGTGCAACAAAACAGTCGAATCTCATGATCTGTTCCATCCTTTTTCTTG | |
| TGTGGTTGAGAAAGGGCAACCACATTCAATCCCTACATCTGCAAATCCTGCCCCTTAG | |
| AOLFR37 sequences: | |
| MEKANETSPVMGFVLLRLSAHPELEKTFFVLILLMYLVILLGNGVLILVTTLDSRLHTPMTTFLG | (SEQ ID NO:69) |
| NLSFLDICFTTSSVPLVLDSFLTPQETISFSACAVQMALSFAMAGTECLLLSMMAFDRYVAICNP | |
| LRYSVIMSKAAYMPMAASSWAIGGAASVVHTSLAIQLPFCGDNYINHFTCEILAVLKLACADIS | |
| INVISMEVTNVIFLGVPVLFISFSYVFIITTILRIPSAEGRIKKVESTCSAIILTVVIVFYGTLFFMYG | |
| KPKSKDSMGADKEDLSDKLIPLFYGVVTPMLNPIIYSLRNKDVKAAVRRTLRPKGFTQ | |
| ATGGAAAAAGGCAATGAGACCTCCCCTGTGATGGGGTTCGTTCTCCTGAGGCTCTCTGCCC | (SEQ ID NO:70) |
| ACCCAGAGCTGGAAAAGACATTCTTCGTGCTCATCCTGCTGATGTACCTCGTGATCCTGCT | |
| GGGCAATGGGGTCCTCATCCTGGTGACCATCCTTGACTCCCGCCTGCACACGCCCATGTAC | |
| TTCTTCCTAGGGAACCTCTCCTTCCTGGACATCTGCTTCACTACCTCCTCAGTCCCACTGGT | |
| CCTGGACAGCTTTTTGACTCCCCAGGAAACCATCTCCTTCTCAGCCTGTGCTGTGCAGATG | |
| GCACTCTCCTTTGCCATGGCAGGAACAGAGTGCTTGCTCCTGAGCATGATGGCATTTGATC | |
| GCTATGTGGCCATCTGCAACCCCCTTAGGTACTCCGTGATCATGAGCTGGCTGCCTACAT | |
| GCCCATGGCTGCCAGCTCCTGGGCTATTGGTGGTGCTGCTTCCGTGGTACACACATCCTTG | |
| GCAATTCAGCTGCCCTTCTGTGGAGACAATGTCATCTCCACTTCACCTGTGAGATTCTGG | |
| CTGTTCTAAGTTGGCCTGTGCTGACATTTCCATCTTGTGATCAGCATGGAGGTGACGAA | |
| TGTGATCTTCCTAGGAGTCCCGGTTCTGTTCATCTCTTTCTCCTATGTCTTCATCATCACCA | |
| CCATCCTGAGGATCCCCTCAGCTGAGGGGAGGAAAAAGGTCTTCTCCACCTGCTCTGCCCA | |
| CCTCACCGTGGTGATCGTCTTCTACGGGACCTTATTCTTCATGTATGGGTGCCTTGTCT | |
| AAGGACTCGATGGGAGCAGACAAAGAGGATCTTTCAGACAAACTCATCCCCCTTTTCTATG | |
| GGGTGGTGACCCCGATGCTCTCCCCATCATCTATAGCCTGAGGAACAAGGATGTGGAAGG | |
| CTGCTGTGAGGAGACTGCTGAGACCAAAAGGCTTCACTCAGTGA | |
| AOLFR38 sequences: | |
| MYLVTVLRNLLIILAVSSDSHLHTPMCFFLSNLCWTIGFTSTTITMQSHSRVISYAGC | (SEQ ID NO:71) |
| LTQMSFFVLFACIEDMLLTVMAYDRFVAICHPLIITUTHLGTLVLVSFFLSLLDSQLHSW | |
| IVLQFTFFKVEISNFVCDPSQLLNLACSDSVTNSIFIYLDSITGFLPISGILLSYANNVPSILRISS | |
| SDRKSKAFSTCGSHLAVVCLFYGTGIGVYLTSAVSPPPRNGVVASVMYAVVTPMLNPFIYSLR | |
| NRDIQSALWRLRSRTVESHDLLSQDLLHPFSCVGEKGQPH | |
| ATGTACCTGGTCACGGTGCTGAGGAACCTGCTCATCATCCTGGCTGTCAGCTCTGACTCCC | (SEQ ID NO:72) |
| ACCTCCACACCCCCATGTGCTTCTTCCTCTCCAACCTGTGCTGGGCTGACATCGGTTTCACC | |
| TCGGCCATGGTTCCCAAGATGATTGTGGACATGCAGTCGCATAGCAGAGTCATCTCTTATG | |
| CGGGCTGCCTGACACAGATGTCTTTCTTTGTCCTTTTTGCATGTATAGTGACATGCTCCTG | |
| ACAGTGATGGCCTATGACCGATTTGTGGCCATCTGTCACCCCCTGCACTACCCAGTCATCA | |
| TGAATCCTCACCTTGGTGTCTTCTTAGTTTTGGTGTCCTTTTTCCTCAGCCTGTTGGATTCC | |
| CAGCTGCACAGTTGGATTGTGTTACAATTCACCTTCTTCAAGAATGTGGAAATCTCCAATT | |
| TTGTCTGTGACCCATCTCAACTTCTCAACCTTGCCTGTTCTGACAGTGTCATCTTAGCATA | |
| TTCATATATTTAGATAGTATTATGTTTGGTTTTCTTCCCATTCAGGGATCCTTTTGTCTTAC | |
| GCTAACAATGTCCCCTCCATTCTAAGAATTTCATCATCAGATAGGTGTCTTAGCCTTCT | |
| CCACCTGTGGCTCTCACCTGGCAGTTGTTTGCTTATTTATGGTCAGGCATTGGCGTGTA | |
| CCTGACTTCAGCTGTGTCACCACCCCCCAGGAATGGTGTGGTGGCATCAGTGATGTACGCT | |
| GTGGTCACCCCCATGCTGAACCCTTTCATCTACAGCCTGAGAAATAGGGAGATTCAAATGTG | |
| CCCTGTGGAGGCTGCGCAGCAGAACAGTCGAATCTCATGATCTGTTATCTCAAGATCTGCT | |
| CCATCCTTTTTCTTGTGTGGGTGAGAAAGGTCAACGACATTTT | |
| AOLFR39 sequences: | |
| MGVKNHSTVTEFLLSGLTEQAELQLPLFCLFLGIYTVTVVGNLSMTSIITTQLHTPMYYFLSS | (SEQ ID NO:73) |
| LSFLDFGYSSVITPKMLSGFLCRDRSISYSGCNIIQLFFFCVCVISECTLTTCDRTAICSPL | |
| LYRVIMSPRVGSLLVAAVFSVGFTDAVIHGGCILRLSFCGSNIICDIVPLITSCSSTYIDEL | |
| LIFVIGGFNMVATSLTIIISYAFILTSILRIHSKKGRCKAESTCSSHLTAVLMFYGSLMSMYLKPAS | |
| SSSLTQEKVSSVFYTTVILMLNPLIYSLRINNEVRNALMKLLRRKISLSPG | |
| ATGGGTGTAAAAAACCATTCCACAGTGACTGAGTTTCTTCTTCAGGATTAACTGAACAAG | (SEQ ID NO:74) |
| CAGAGCTTCAGCTGCCCCTCTTCTGCCTCTTCTAGGAATTACACAGTTACTGTGGTGGG | |
| AAACCTCAGCATGATCTCAATTATTAGGCTGAATCGTCAACTTCATACCCCCATGTACTAT | |
| TTCCTGAGTAGTTTGTCTTTTTAGATTTCTGCTATTCTTCTGTCATTACCCCTAAAATGCT | |
| ATCAGGGTTTTTATGCAGAGATAGATCCATCTCCTATTCTGGATGCATGATTCAGCTGTTT | |
| TTTTCTGTGTTTGTGTTATTTCTGAATGCTACATGCTGGCAGCCATGGCCTGCGATCGCTAC | |
| GTGGCCATCTGGAGCCCACTGCTCTAGAGGGTCATCATGTCGCCTAGGGTCTGTTCTCTGC | |
| TGGTGGGTGCTGTCTTCTCAGTAGGTTTCACTGATGCTGTGATCCATGGAGGTTGTATACT | |
| CAGGTTGTCTTTCTGTGGATCAAACATCATTAAACATTATTTCTGTGACATTGTCCCTCTTA | |
| TTAAACTCTCCTGCTCCAGCACTTATATTGATGAGCTTTTGATTTTGTCATTGGTGGATTT | |
| AACATGGTGGCCACAAGCCTAACAATCATTATTTCATATGCTTTTATCCTCACCAGCATCCT | |
| GCGCATCCACTCTAAAAAGGGCAGGTGCAAAGCGTTTAGCACCTGTAGCTCCCACCTGACA | |
| GCTGTTCTTATGTTTTATGGGTCTCTGATGTCCATGTATCTCTCAAACCTGCTTCTAGCAGTTC | |
| ACTCACCCAGGAGAAAGTATCCTCAGTATTTTATACCACTGTGATTCTCATGTTGAACCC | |
| TTGATATATAGTCTGAGGACAATGAAGTAAGAAATGCTCTGATGAAACTTTTAAGAAGA | |
| AAAATATCTTTATCTCCAGGATCC | |
| AOLFR40 sequences: | |
| MSNATLLTAFILTGLPHAPGLDAPLFGIFLVVYVLGNLLILLVIRVDSHLHTPMYYFLTNLS | (SEQ ID NO:75) |
| FIDMWFSTVTVPKMLMTLVSPSGRTISFHSCVAQLYFFHFLGSTECFLYTVMSYDRYLAISYPL | |
| RYTNMMTGRSCALLATGTWLSGSLHSAVQTILTFHLPYCGPNQIQHYFCDAPPILKLACADTS | |
| ANEMVIFVNIGLVASGCFVLIVLSYVSIVCSILRIRTSEGRHRAFQTCASHCIVVLCFFGPGLFIYL | |
| RPGSRDALHGVVAVFYTTLTPLFNPVVYTLRNKEVKKALLKLNGSVFAQGE | |
| ATGTCCAACGCCACCCTACTGACAGCGTTCATCCTCACGGGCCTTCCCCATGCCCCAGGGC | (SEQ ID NO:76) |
| TGGACGCCCCCCTCTTTGGAATGTTCCTGGTGGTTTACGTGCTCACTGTGCTGGGGAACCT | |
| CCTCATCCTGCTGGTGATCAGGGTGGATTCTCACCTCCACACCCCCATGTACTACTTCCTCA | |
| CCACCTGTCCTTCATTGACATGTGGTTCTCCACTGTCACGGTGCCCAAAATGTGATGAC | |
| CTTGGTGTCCCCAAGCGGCAGGACTATCTCCTTCCACAGCTGCGTGGCTCAGCTCTATTTTT | |
| TCCACTTCCTGGGGAGCACCGAGTGTTTCCTCTACACAGTCATGTCCTATGATCGCTACCT | |
| GGCCATCAGTTACCCGCTCAGGTACACCAACATGATGACTGGGCGCTCGTGTGCCCTCCTG | |
| GCCACCGGCACTTGCTCAGTGGCTCTCTGCACTCTGCTGTCCAGACCATATTGACTTTCC | |
| ATTTGCCCTACTGTGGACCCAACCAGATCCAGCACTACTTCTGTGACGCACCGCCCATCCT | |
| GAAACTGGCCTGTGCAGACACCTCAGCCAACGAGATGGTCATCCTTTGTGAATATTGGGCTA | |
| GTGGCCTCGGGCTGCTTTGTCCGATAGTGCTGTCCTATGTGTCCATCGTCTGTTCCATCCT | |
| GCGGATCCGCACCTCAGAGGGGAGGCACAGAGCCTTTCAGACCTGTGCCTCCCACTGTATC | |
| GTGGTCCTTTGCTTCTTTGGCCCTGGTCTTTTCATTTACCTGAGGCCAGGCTCCAGGGACGC | |
| CTTGCATGGGGTTGTGGCCGTTTTCTACACCACGCTGACTCCTCTTTTCAACCCTGTTGTGT | |
| ACACCCTGAGAAACAAGGAGGTAAAGAAAGCTCTGTTGAAGCTGAAAAATGGGTCAGTAT | |
| TTGCTCAGGGTGAATAG | |
| AOFR41 sequences: | |
| MNPENWTQVTSFVLLGFPSSHLIQFLVFLGLMVTYIVTATGKLLIIVLSWIDQRLHIQMYFFLRN | (SEQ ID NO:77) |
| FSFLELLLVTVVVPKMLVVILTGDHTISFVSCIIQSYLYFFLGTTDFFLLAVMSLDRYLAICRPLR | |
| YETLMNGHVCSQLVLASWLAGFLWVLCPTVLMASLPFCGPNGIDHFFRDSWPLLRLSCGDTH | |
| LLKLVAFMLSTLVLLGSLALTSVSYACILATVLRAPTAAERRKAFSTCASHLTVVVIIYGSSIFLY | |
| IRMSEAQSKLLNKGASVLSCIITPLLNPFIFTLRNDKQQALREALGWPRLTAVMKLRVTSQRK | |
| ATGAACCCTGAAAACTGGACTCAGGTAACAAGCTTTGTCCTTCTGGGTTTCCCCAGTAGCC | (SEQ ID NO:78) |
| ACCTCATACAGTTCCTGGTGTTCCTGGGGTTAATGGTGACCTACATTGTAACAGCCACAGG | |
| CAAGCTGCTAATTATTGTGCTCAGCTGGATAGACCAACGCCTGCACATACAGATGTACTTC | |
| TTCTGCGGAATTTCTCCTTCCTGGAGCTGTTGCTGGTAACTGTTGTGGTTCCCAAGATGCT | |
| TGTCGTCATCCTCACGGGGGATCACACCATCTCATTTGTCAGCTGCATCATCCAGTCCTACC | |
| TCTACTTCTTTCTAGGCACCACTGACTTCTTCCTCTTGGCCGTCATGTCTCTGGATCGTTAC | |
| CTGGCAATCTGCCGACCACTCCGCTATGAGACCCTGATGAATGGCCATGTCTGTTCCCAAC | |
| TAGTGCTGGCCTCCTGGCTAGCTGGATTCCTCTGGGTCCTTTGCCCCACTGTCCTCATGGCC | |
| AGCCTGCCTTTCTGTGGCCCCAATGGTATTGACCACTTCTTTCGTGACAGTTGGCCCTTGCT | |
| CAGGCTTTCTTGTGGGGACACCCACCTGCTGAAACTGGTGGCTTTCATGCTCTCTACGTTG | |
| GTGTTACTGGGCTCACTGGCTCTGACCTCAGTTTCCTATGCCTGCATTCTTGCCACTGTTCT | |
| CAGGCCCCTACAGCTGCTGAGCGAAGGAAAGCGTTTTCCACTTGCGCCTCGCATCTTACA | |
| GTGGTGGTCATCATCTATGGCAGTTCCATCTTTCTCTACATTCGTATGTCAGAGGCTCAGTC | |
| CAAACTGCTCAACAAAGGTGCCTCCGTCCTGAGCTGCATCATCACACCCCTCTTGAACCCA | |
| TTCATCTTCACTCTCCGCAATGACAAGGTGCAGCAAGCACTGAGAGAAGCCTTGGGGTGGC | |
| CCAGGCTCACTGCTGTGATGAAACTGAGGGTCACAAGTCAAAGGAAATGA | |
| AOLFR42 sequences: | |
| MNPANHSQVAGFVLLGLSQVWELRFVFFTVFSAVYFMTVVGNLLIVVIVTSDPHLHTTMYFLL | (SEQ ID NO:79) |
| GNLSFLDFCYSSITAPRMLVDLLSGNPTISFGGCLTQLFFFHFIGGIKIFLLTVMAYDRYIAISQPL | |
| HYTLIMNQTVCALLMAASWVGGFIHSIVQIALTIQLPFCGPKLDNFYCDVPQLIKLACTDTFV | |
| LELLMVSNNGLVTLMCFLVLLGSYTALLVMLRSKALSTCASHIAVVTLIFVPCIYVY | |
| TRPFRTFPMDKAVSVLYTIVTPMLNPAIYTLRNKEVIMAMKKLWRRKKDPIGPLEHRPLH | |
| ATGAATCCAGCAAATCATTCCCAGGTGGCAGGATTTGTTCTACTGGGGCTCTCTCAGGTTT | (SEQ ID NO:80) |
| GGGAGCTTCGGTTTGTTTTCTTCACTGTTTTCTCTGCTGTGTATTTTATGACTGTAGTGGGA | |
| AACCTCTTATTGTGGTCATAGTGACCTCCGACCCACACCTGCACACAACCATGTATTTTCT | |
| CTTGGGCAATCTTTCTTTCCTGGACTTTTGCTACTCTTCATCACAGCACCTAGGATGCTGG | |
| TTGACTTGCTCTCAGGCAACCCTACCATTTCCTTTGGTGGATGCCTGACTCAACTCTTCTTC | |
| TTCCACTTCATTGGAGGCATCAAGATCTTCCTGCTGACTGTCATGGCGTATGACCGCTACA | |
| TTGCCATTTCCCAGCCCCTGCACTACACGCTCATTATGAATCAGACTGTCTGTGCACTCCTT | |
| ATGGCAGCCTCCTGGGTGGGGGGCTTCATCCACTCCATAGTACAGATTGCATTGACTATCC | |
| AGCTGCCATTCTGTGGGCCTGACAAGCTGGACAACTTTTATTGTGATGTGCCTCAGCTGAT | |
| CAAATTGGCCTGCACAGATACCTTTGTCTTAGAGCTTTTAATGGTGTCTAACAATGGCCTG | |
| GTGACCCGATGTGTTTTCTGGTGCTTCTGGGATCGTACACAGCACTGCTAGTCATGCTCC | |
| GAAGCCACTCACGGGAGGGCCGCAGCAAGGCCCTGTCTACCTGTGCCTCTCACATTGCTGT | |
| GGTGACCTTAATCTTTGTGCCTTGCATCTACGTCTATACAAGGCCTTTTCGGACATTCCCCA | |
| TGGACAAGGCCGTCTCTGTGCTATACACAATTGTCACCCCCATGCTGAATCCTGCCATCTA | |
| TACCCTGAGAAACAAGGAAGTGATCATGGCCATGAAGAAGCTGTGGAGGAGGAAAAAGG | |
| ACCCTATTGGTCCCCTGGAGCACAGACCCTTACATTAG | |
| AOLFR43 sequences: | |
| MQKPQLLVPIIATSNGNLVHAAYFLLVGIPGLGPTIHFWLAFPLCFMYALATLGNLTIVLIIRVE | (SEQ ID NO:81) |
| RRLHEPMYLFLAMLSTIDLVLSSITMPKMASLFLMGIQEIEFNICLAQMFLIHALSAVESAVLLA | |
| MAFDRFVAICHPLRHASVLTGCTVAKIGLSALTRGFVFFFPLPFILKWLSYCQTHTVTHSFCLHQ | |
| DIMKLSCTDTRVNVVYGLFIILSVMGVDSLFIGFSYILILWAVLELSSRRAALKAFNTCISHLCAV | |
| LVFYVPLIGLSVVHRLGGPTSLLHVVMANTYLLLPPVVNPLVYGAKTKEICSRVLCMFSQGGK | |
| ATGCAGAAGCCCCAGCTCTTGGTCCCTATCATAGCCACTTCAAATGGAAATCTGGTCCACG | (SEQ ID NO:82) |
| CAGCATACTTCCTTTTGGTGGGTATCCCTGGCCTGGGGCCTACCATACACTTTTGGCTGGCT | |
| TTCCCACTGTGTTTTATGTATGCCTTGGCCACCCTGGGTAACCTGACCATTGTCCTCATCAT | |
| TCGTGTGGAGAGGCGACTGCATGAGCCCATGTACCTCTTCCTGGCCATGCTTTCCACTATT | |
| GACCTAGTCCTCTCCTCTATCACCATGCCCAAGATGGCCAGTCTTTTCCTGATGGGCATCCA | |
| GGAGATCGAGTTCAACATTTGCCTGGCCCAGATGTTCCTTATCCATGCTCTGTCAGCCGTG | |
| GAGTCAGCTGTCCTGCTGGCCATGGCTTTTGACCGCTTTGTGGCCATTTGCCACCCATTGC | |
| GCCATGCTTCTGTGCTGACAGGGTGTACTGTGGCCAAGATTGGACTATCTGCCCTGACCAG | |
| GGGGTTTGTATTCTTCTTCCCACTGCCCTTCATCCTCAAGTGGTTGTCCTACTGCCAAACAC | |
| ATACTGTCACACACTCCTTCTGTCTGCACCAAGATATTATGAAGCTGTCCTGTACTGACAC | |
| CAGGGTCAATGTGGTTTATGGACTCTTCATCATCCTCTCAGTCATGGGTGTGGACTCTCTCT | |
| TCATTGGCTTCTCATATATCCTCATCCTGTGGGCTGTTTTGGAGCTGTCCTCTCGGAGGGCA | |
| GCACTCAAGGCTTTCAACACCTGCATCTCCCACCTCTGTGCTGTTCTGGTCTTCTATGTACC | |
| CCTCATTGGGCTCTCGGTGGTGCATAGGCTGGGTGGTCCCACCTCCCTCCTCCATGTGGTT | |
| ATGGCTAATACCTACTTGCTGCTACCACCTGTAGTCAACCCCCTTGTCTATGGAGCCAAGA | |
| CCAAAGAGATCTGTTCAAGGGTCCTCTGTATGTTCTCACAAGGTGGCAAGTGA | |
| AOLFR44 sequences: | |
| MSSCNFTHATFVLIGIPGLEKAHFWVGFPLLSMYVVAMFGNCIVVFIVRTERSLHAPMYLFLC | (SEQ ID NO:83) |
| MLAAIDLALSTSTMPKILALFWFDSREISFEACLTQMFFIHALSAIESTILLAMAFDRYVAICHPL | |
| RHAAVLNNTVTAQIGIVAVVRGSLFFFPLPLLIKRLAFCHSNVLSHSYCVHQDVMKLAYADTLP | |
| NVVYGLTAILLVMGVDVMFISLSYFLIIRTVLQLPSKSERAKAFGTCVSHIGVVLAFYVPLIGLS | |
| VVHRFGNSLHPIVRVVMGDIYLLLPPVINPIIYGAKTKQIRTRVLAMFKISCDKDLQAVGGK | |
| ATGAGTTCCTGCAACTTCACACATGCCACCTTTGTGCTTATTGGTATCCCAGGATTAGAGA | (SEQ ID NO:84) |
| AAGCCCATTTCTGGGTTGGCTTCCCCCTCCTTTCCATGTATGTAGTGGCAATGTTTGGAAAC | |
| TGCATCGTGGTCTTCATCGTAAGGACGGAACGCAGCCTGCACGCTCCGATGTACCTCTTTC | |
| TCTGCATGCTTGCAGCCATTGACCTGGCCTTATCCACATCCACCATGCCTAAGATCCTTGCC | |
| CTTTTCTGGTTTGATTCCCGAGAGATTAGCTTTGAGGCCTGTCTTACCCAGATGTTCTTTAT | |
| TCATGCCCTCTCAGCCATTGAATCCACCATCCTGCTGGCCATGGCCTTTGACCGTTATGTGG | |
| CCATCTGCCACCCACTGCGCCATGCTGCAGTGCTCAACAATACAGTAACAGCCCAGATTGG | |
| CATCGTGGCTGTGGTCCGCGGATCCCTCTTTTTTTTCCCACTGCCTCTGCTGATCAAGCGGC | |
| TGGCCTTCTGCCACTCCAATGTCCTCTCGCACTCCTATTGTGTCCACCAGGATGTAATGAA | |
| GTTGGCCTATGCAGACACTTTGCCCAATGTGGTATATGGTCTTACTGCCATTCTGCTGGTC | |
| ATGGGCGTGGACGTAATGTTCATCTCCTTGTCCTATTTTCTGATAATACGAACGGTTCTGC | |
| AACTGCCTTCCAAGTCAGAGCGGGCCAAGGCCTTTGGAACCTGTGTGTCACACATTGGTGT | |
| GGTACTCGCCTTCTATGTGCCACTTATTGGCCTCTCAGTGGTACACCGCTTTGGAAACAGC | |
| CTTCATCCCATTGTGCGTGTTGTCATGGGTGACATCTACCTGCTGCTGCCTCCTGTCATCAA | |
| TCCCATCATCTATGGTGCCAAAACCAAACAGATCAGAACACGGGTGCTGGCTATGTTCAAG | |
| ATCAGCTGTGACAAGGACTTGCAGGCTGTGGGAGGCAAGTGA | |
| AOLFR45 sequences: | |
| MLPSNITSTHPAVFLLVGIPGLEHLHAWISIPFCFAYTLALLGNCTLLFIIQADAALHEPMYLFLA | (SEQ ID NO:85) |
| MLATIDLVLSSTTLPKMLAIFWFRDQEINFFLHSFSIMESAVLLAMAFDRYVAICKP | |
| LHYTTVLTGSLITKIGMAAVARAVTLMTPLPFLLRRFHYCRGPVIAHCYCEHMAVVRLACGDT | |
| SFNNIYGIAVAMFSVVLDLLFVILSYVFILQAVLQLASQEARYKAFGTCVSHIGAILSTYTPVVIS | |
| SVMHRVARHAAPRVHILLAIFYLLFPPMVNPIIYGVKTKQIREYVLSLFQRKNM | |
| TGGAAACAAGAGGTAATCTTTGCAGGTGGGATAGCACAGGTTGAACTCTAATCATATATA | (SEQ ID NO:86) |
| CTGTAGAAGGTATATATAGAAGGTGAAGAAGCCCTGTAAAAATTGACAAGGAGATTTCCA | |
| GGAGCCATGCTTCCCTCTAATATCACCTCAACACATCCAGCTGTCTTTTTGTTGGTAGGAAT | |
| TCCGGTTTGGAACACCTGCATGCCTGGATCTCCATCCCCTTCTGCTTTGCTTATACTCTGG | |
| CCCTGCTAGGCAACTGTACCCTTCTCTTCATTATCCAGGCTGATGCAGCCCTCCATGAACCC | |
| ATGTACCTCTTTCTGGCCATGTTGGCAACCATTGACTTGGTTCTTTCTTCTACAACGCTGCC | |
| CAAAATGCTGCCATATTCTGGTTCAGGGATCAGGAGATCAACTTCTTTGCCTGTCTGGTC | |
| CAGATGTTCTTCCTTCACTCCTTCTCCATCATGGAGTCAGCAGTGCTGCTGGCCATGGCCTT | |
| TGACCGCTATGTGGCCATCTGCAAGCCATTGCACTACACGACGGTCCTGACTGGGTCCCTC | |
| ATCACCAAGATTGGCATGGCTGCTGTGGCCCGGGCTGTGACACTAATGACTCCACTCCCCT | |
| TCCGCTCAGACGCTTCCACTACTGCCGAGGCCCAGTGATTGCCCATTGCTACTGTGAACA | |
| CATGGCTGTGGTAAGGCTGGCGTGTGGGGACACTAGCTTCAACAATATCTATGGCATTGCT | |
| GGTGGCCATGTTTAGTGTGGTGTTGGACCTGCTCTTTGTTATCCTGTCTTATGTCTTCATCCT | |
| TCAGGCAGTTCTCCAGCTTGCCTCTCAGGAGGCCCGCTACAAAGCATTTGGGACATGTGTG | |
| TCTCACATAGGTGCCATCCTGTCCACCTACACTCCAGTAGTCATCTCTTCAGTCATGCACCG | |
| TGTAGCCCGCCATGCTGCCCCTCGTGTCCACATACTCCTTGCTATTTTCTATCTCCTTTTCC | |
| CACCCATGGTCAATCCTATCATATATGGAGTCAAGACCAAGCAGATTCGTGAGTATGTGCT | |
| CAGTCTATTCCAGAGAAAGAACATGTAGATGGATAGTTCTCTTTTTTTATCCCACTTGCCA | |
| AGTAATGAGAATGCTGGATTGGGGTTGAGGGGAAAAATCTAAATAGGAAAATTGCAGAGT | |
| ATCTTTGACAATTCTCTAGTATGATAAGGAAAATGAGGTTTCATTCCTCACAGATCTACGA | |
| GTCAGGTCAAACCAGGAGTGCACCTATAGTCTGGTCTGATAGTAGAGGTTTGACCTTCCCA | |
| TTGTCATAGACTCATCACATGGCTAAGGAAGACAAACCTCTCAAAGTGGTATTGTAATCTG | |
| GGTGAAAGACAGTAGGACCTTTATTGGCTGAGATTGGCCCAAACAGCTGAGTC | |
| AOLFR46 sequences: | |
| MNIKHCGWHMIHTWLNIREDDDSDFKNFIGQIQGLSGNPHSTTSRMYFLCFCTSLLGFKVHWV | (SEQ ID NO:87) |
| SRLIXKLYMASPNNDSTAPVSEFLLICFPNFQSWQHWLSLPLSLLFLLAMGANTTLLITIQLEAS | |
| LHQPLYYLLSLLSLLDIVLCLTVIPKVLAIFWFDLRSISFPACEFLQMFIMNSFLTMESCTFMVMA | |
| YDRYVAICHPLRYPSIITDQFVARAVVFVIARNAFVSLPVPMLSARLRYCAGNIIKNCICSNLSVS | |
| KLSCDDITFNQLYFVAGWTLLGSDLILIVISYSFILKVVLRIKAEGAVAKALSTCGSHFILILFFS | |
| TVLLVLVITNLARKRIPPDVPILLNILHHLIPPALNPIVYGVRTKEIKQGIQNLLKRL | |
| ATGAATATAAAACATTGTGGCTGGCATATGATACATACTTGGTTAAATATAAGGGAGGAT | (SEQ ID NO:88) |
| GATGACAGTGATTTTAAAAACTTTATTGGACAGATACAGGGCCTCAGTGGAAACCCACACT | |
| CTACTACGTCTAGAATGTACTTTTTATGTTTCTGTACTTCTCTACTAGGTTTTAAGGTACAC | |
| TGGTCTCCAGATTGATCANGAAACTTTACATGGCATCTCCCAACAATGACTCCACTGCCC | |
| CAGTCTCTGAATTCCTCCTCATCTGCTTCCCCAACTTCCAGAGCTGGCAGCACTGGTTGTCT | |
| CTGCCCCTCAGCCTTCTCTTCCTCCTGGCCATGGGAGCTAACACCACCCTCCTGATCACCAT | |
| CCAGCTGGAGGCCTCTCTGCACCAGCCCCTGTACTACCTGCTCAGCCTCCTCTCCCTGCTGG | |
| ACATCGTGCTCTGCCTCACCGTCATCCCCAAGGTCCTGGCCATCTTCTGGTTTGACCTCAGG | |
| TCGATCAGCTTCCCAGCCTGCTTCCTCCAGATGTTCATCATGAACAGTTTTTTGACCATGGA | |
| GTCCTGCACGTTCATGGTCATGGCCTATGACCGTTATGTGGCCATCTGCCATCCATGAGA | |
| TACCCGTCTATCATCACTGACCAGTTTGTGGCTAGGGCCGTGGTCTTTGTTATAGCCCGGA | |
| ATGCCTTTGTTTCTCTTCCTGTTCCCATGCTTTCTGCCAGGCTCAGATACTGTGCAGGAAAC | |
| ATAATCAAGAACTGCATCTGCAGTAACCTGTCTGTGTCCAAACTCTCTTGTGATGACATCA | |
| CTTTCAATCAGCTCTACCAGTTTGTGGCAGGCTGGACTCTGTTGGGCTCTGATCTTATCCTT | |
| ATTGTTATCTCCTATTCTTTTATATTGAAAGTTGTGCTTAGGATCAAGGCCGAGGGTGCGT | |
| GGCCAAGGCCTTGAGCACGTGTGGTTCCCACTTCATCCTCATCCTCTTCTTCAGCACAGTCC | |
| TGCTGGTTCTGGTCATCACTAACCTGGCCAGGAAGAGAATTCCTCCAGATGTCCCCATCCT | |
| GCTCAACATCCTGCACCACCTCATTCCCCCAGCTCTGAACCCCATTGTTTATGGTGTGAGA | |
| ACCAAGGAGATCAAGCAGGGAATCCAAAACCTGCTGAAGAGGTTGTAA | |
| AOLFR47 sequences: | |
| MSAASNITLTHPTAFLLVGIPGLEHLHIWISIPFCLAYTLALLGNCTLLLIIQADAALHEPMYLFLA | (SEQ ID NO:89) |
| MLAAIDLVLSSSALPKMLAIFWFRDREINFFACLAQMFFLHSFSIMESAVLLAMAFDRYVAICK | |
| PLHYTKVLTGSLITKIGMAAVARAVTLMTPLPFLLRCFHYCRGPVIAHCYCEHMAVVRLACGD | |
| TSFNNIYGIAVAMFIVVLDLLLVILSYIFILQAVLLLASQEARYKAFGTCVSHIGAILAFYTTVVIS | |
| SVMHRVARHAAPHVHILLANFYLLFPPMVNPIIYGVKTKQIRESILGVKTKQIRESILGVFPRKDM | |
| ATGTCAGCCTCCAATATCACCTTAACACATCCAACTGCCTTTCTTGTTGTGGGGATTCCAG | (SEQ ID NO:90) |
| GCCTGGACACCTGCACATCTGGATCTCCATCCCTTTCTGCTTAGCATATACACTGGCCCTG | |
| CTTGGAAACTGCACTCTCCTTCTCATCATCCAGGCTGATGCAGCCCTCCATGAACCCATGT | |
| ACCTCTTTCTGGCCATGTGGCAGCCATCGACCTGGTCCTTTCCTCCTCAGCACTGCCCAAA | |
| ATGCTTGCCATATTCTGGTTCAGGGATCGGGAGATAAACTTCTTTGCCTGTCTGGCCCAGA | |
| TGTTCTTCCTTCACTCCTTCTCCATCATGGAGTCAGCAGTGCTGCTGGCCATGGCCTTTGAC | |
| CGATATGTGGCTATCTGCAAGCCACTGCACTACACCAAGGTCCTGACTGGGTCCCTCATCA | |
| CCAAGATTGGCATGGCTGCTGTGGCCCGGGCTGTGACACTAATGACTCCACTCCCCTTCCT | |
| GCTGAGATGTTTCCACTACTGCCGAGGCCCAGTGATCGCTCACTGCTACTGTGAACACATG | |
| GCTGTGGTGAGGCTGGCGTGTGGGGACACTAGCTTCAACAATATCTATGGCATCGCTGTGG | |
| CCATGTTTATTGTGGTGTTGGACCTGCTCCTTGTTATCCTGTCTTATATCTTTATTCTTCAG | |
| GCAGTTCTACTGCTTGCCTCTCAGGAGGCCCGCTACAAGGCATTTGGGACATGTGTCTCTC | |
| ATATAGGTGCCATCTTAGCCTTCTACACAACTGTGGTCATCTCTTCAGTCATGCACCGTGTA | |
| GCCCGCCATGCTGCCCCTCATGTCCACATCCTCCTTGCCAATTTCTATCTGCTCTTCCCACC | |
| CATGGTCAATCCCATAATCTATGGTGTCAAGACCAAGCAAATCCGTGAGAGCATCTTGGGA | |
| GTATTCCCAAGAAAGGATATGTAG | |
| AOLFR48 sequences: | |
| MMVDPNGNESSATYFILIGLPGLEEAQFWLAFPLCSLYLIAVLGNLTIIYIVRTEHSLHEPMYIFL | (SEQ ID NO:91) |
| CMLSGIDILISTSSMPKMLAIFWFNSTTIQFDACLLQMFAIHSLSGMESTVLLAMAFDRYVAICH | |
| PLRHATVLTLPRVTKIGVAAVVRGAALMAPLPVFIKQLPFCRSNILSHSYCLHDVMKLACDDI | |
| RVNVVYGLIVIISAIGLDSLLISFSYLLILKTVLGLTREAQAKAFGTCVSHVCAVFIFYVPFIGLSM | |
| VHRFSKRRDSPLPVILANIYLLVPPVLNPIVYGVKTKEIRQRILRLFHVATHASEP | |
| ATGATGGTGGATCCCAATGGCAATGAATCCAGTGCTACATACTTCATCCTAATAGGCCTCC | (SEQ ID NO:92) |
| CTGGTTTAGAAGAGGCTCAGTTCTGGTTGGCCTTCCCATTGTGCTCCCTCTACCTTATTGCT | |
| GTGCTAGGTAACTTGACAATCATCTACATTGTGCGGACTGAGCACAGCCTGCATGAGCCCA | |
| TGTATATATTTCTTTGCATGCTTTCAGGCATTGACATCCTCATCTCCACCTCATCCATGCCC | |
| AAAATGCTGGCCATCTTCTGGTTCAATCCACTACCATCCAGTTTTGATGCTTGTCTGCTACA | |
| GATGTTTGCCATCCACTCCTTATCTGGCATGGAATCCACAGTGCTGCTGGCCATGGCTTTT | |
| GACCGCTATGTGGCCATCTGTCACCCACTGCGCCATGCCACAGTACTTACGTTGCCTCGTG | |
| TCACCAAAATTGGTGTGGCTGCTGTGGTGCGGGGGGGCTGCACTGATGGCACCCCTTCCTGT | |
| CTTCATCAAGCAGCTGCCCTTCTGCCGCTCCAATATCCTTTCCCATTCCTACTGCCTACACC | |
| AAGATGTCATGAAGCTGGCCTGTGATGATATCCGGGTCAATGTCGTCTATGGCCTTATCGT | |
| CATCATCTCCGCCATTGGCCTGGACTCACTTCTCATCTCCTTCTCATATCTGCTTATTCTTA | |
| AGACTGTGTTGGGCTTGACACGTGAAGCCCAGGCCAAGGCATTTGGCACTTGCGTCTCTCA | |
| TGTGTGTGCTGTGTTCATATTCTAT5GTACCTTTCATTGGATTGTCCATGGTGCATCGCTTTA | |
| GCAAGCGGCGTGACTCTCCGCTGCCCGTCATCTTGGCCAATATCTATCTGCTGGTTCCTCCT | |
| GTGCTCAACCCAATTGTCTATGGAGTGAAGACAAAGGAGATTCGACAGCGCATCCTTCGA | |
| CTTTTCCATGTGGCCACACACGCTTCAGAGCCCTAG | |
| AOLFR49 sequences: | |
| MTFHNVCSPSSFWLTGIPGLESLHVWLSIPFGSMYLVAVVGNVTILAVVKIERSLHQPMYFF | (SEQ ID NO:93) |
| LCMLAAIDLVLSTSTIPKLLGIFWFGACDIGDACLGQMFLIHCFATVESGIFLAMAFDRYVAIC | |
| NPLRHSMVLTYTVVGRLGLVSLLRGVLYIGPLPLMIRLPLYKTHVISHSYCEHMAVVALTC | |
| GDSRVNNVYGLSIGFLVLILDSVAIAASYVMIFRAVMGLATPEARLKTLGTCASHLCAILIFYVP | |
| IAVSSLIHRFGQCVPPPVHTLLANFYLLIPPILNPIVYAVRTKQIRESLLQIPRIEMKIR | |
| ATGCTCACTTTTCATAATGTCTGCTCAGTACCCAGCTCCTTCTGGCTCACTGGCATCCCAGG | (SEQ ID NO:94) |
| GCTGGAGTCCCTACACGTCTGGCTCTCCATCCCCTTTGCTCCATGTACCTGGTGGCTGTG | |
| GTGGGGAATGTGACCATCCTGGCTGTGGTAAAGATAGAACGCAGCCTGCACCAGCCCATG | |
| TACTTTTTCTTGTGCATGTTGGCTGCCATTGACCTGGTTCTGTCTACTTCCACTATACCCAA | |
| ACTTCTGGGAATCTTCTGGTTCGGTGCTTGTGACATTGGCCTGGACGCCTGCTTGGGCCAA | |
| ATGTTCCTTATCCACTGCTTTGCCACTGTTGAGTCAGGCATCTTCCTTGCCATGGCTTTTGA | |
| TCGCTACGTGGCCATCTGCAACCCACTACGTCATAGCATGGTGCTCACTTATACAGTGGTG | |
| GGTCGTTTGGGGCTTGTTTCTCTCCTCCGGGGTGTTCTCTACATTGGACCTCTGCCTCTGAT | |
| GATCCGCCTGCGGCTGCCCCTTTATAAAACCCATGTTATCTCCCACTCCTACTGTGAGCAC | |
| ATGGCTGTAGTTGCCTTGACATGTGGCGACAGCAGGGTCAATAATGTCTATGGGCTGAGC | |
| ATCGGCTTTCTGGTGTTGATCCTGGACTCAGTGGCTATTGCTGCATCCTATGTGATGATTTT | |
| CAGGGCCGTGTATGGGGTTAGCCACTCCTGAGGCTAGGCTTAAAACCCTGGGGACATGCGC | |
| TTCTCACCTCTGTGCCATCCTGATCTTTTATGTTCCCATTGCTGTTTCTTCCCTGATTCACCG | |
| ATTTGGTCAGTGTGTGCCTCCTCCAGTCCACACTCTGCTGGCCAACTTCTATCTCCTCATTC | |
| CTCCAATCCTCAATCCCATTGTCTATGCTGTTCGCACCAAGCAGATCCGAGAGAGCCTTCT | |
| CCAAATACCAAGGATAGAAATGAAGATTAGATGA | |
| AOLFR50 sequences: | |
| MNLDSFFSFLLKSLIIMALSNSSWRLPQPSFFLVGIPGLEESQHWIALPLGILYLLALVGNVTILFII | (SEQ ID NO:95) |
| WMDPSLHQSMYLFLSMLAAIDLVVASSTAPKALAVLLVRAQEIGYTVCLIQMFFTHAFSSMES | |
| GVLVAMALDRYVAICHPLHHSTILHPGVIGHIGMVVLVRGLLLLIPFLILLRKLIFCQATIIGHAY | |
| CEHMAVVKLACSETTVNRAYGLTVALLAAGLDVLAIGVSYAHILQAVLKVPGNEARLKAFST | |
| CGSHVCVILVFYIPGMFSFLTHRFGHHVPHHVHVLLAILYRLVPPALNPLVYRVKTQIHQ | |
| ATGAATTTGGATTCTTTTTTCTCTTTCCTCCTCAAGTCATTGATAATGGCACTTAGCAATTC | (SEQ ID NO:96) |
| CAGCTGGAGGCTACCCCAGCCTTCTTTTTTCCTGGTAGGAATTCCGGGTTTAGAGGAAAGC | |
| CAGACTGGATCGCACTGCCCCTGGGCATCCTTTACCTCCTTGCTCTAGTGGGCAATGTTA | |
| CCATTCTCTTCATCATCTGGATGGACCCATCCTTGCACCAATCTATGACCTCTTCCTGTCC | |
| ATGCTAGCTGCCATCGACCTGGTTGTGGCCTCCTCCACTGCACCCAAAGCCCTTGCAGTGC | |
| TCCTGGTTCGTGCCCAAGAGATTGGTTACACTGTCTGCCTGATCCAGATGTTCTTCACCCAT | |
| GCATTCTCCTCCATGGAGTCAGGGGTACTTGTGGCCATGGCTCTGGATCGCTATGTAGCCA | |
| TTTGTCACCCCTTGCACCATTCCACAATCCTGCATCCAGGGGTCATAGGGCACATCGGAAT | |
| GGTGGTGCTGGTGCGGGGATTACTACTCCTCATCCCCTTCCTCATTCTGTTGCGAAAACTT | |
| ATCTTCTGCCAAGCCACCATCATAGGCCATGCCTATTGTGAACATATGGCTGTTGTGAAAC | |
| TGCCTGCTCAGAAACCACAGTCAATCGAGCTTATGGGCTGACTGTGGCCTTGCTTGTGGT | |
| TGGGCTGGATGTCCTGGCCATTGGTGTTTCCTATGCCCACATTCTCCAGGCAGTGCTGAAG | |
| GTACCAGGAAATGAGGCCCGACTTAAGGCCTTTAGCACATGTGGCTCTCATGTTTGTGTCA | |
| TCCTGGTCTTCTATATCCCGGGAATGTTCTCCTTCCTCACTCACCGCTTTGGTCATCATGTA | |
| CCCCATCACGTCCATGTTCTTCTGGCCATACTGTATCGCCTTGTGCCACCTGCACTCAATCC | |
| TCTTGTCTATAGGGTGAAGACCCAGAAGATCCACCAGTGA | |
| AOLFR51 sequences: | |
| MCQQILRDCILLIHHLCINRKKVSLVMLGPAYNHTMETPASFLLVGIPGLQSSHLWLAISLSAM | |
| YIIALLGNTIIVTAIWMDSTRHEPMYCFLCVLAAVDIVMASSVVPKMVSIFCSGDSSISFSACFTQ | |
| MFFVHLATAVETGLLLTMAFDRYVAICKPLHYKRILTPQVMLGMSMAITIRAILAITPLSWMVS | |
| HLPFCGSNVVVHSYCEHIALARLACADPVPSSLYSLIGSSLMVGSDVAFIAASYILILKAVFGLSS | |
| KTAQLKALSTCGSHVGVMALYYLPGMASIYAAWLGQDVPLHTQVLLADLYVIIPATLNPIIY | |
| GMRTKQLRERIWSYLMHVLFDHSNLGS | |
| ATGTGTCAACAAATCTTACGGGATTGCATTCTTCTCATACATCATTTGTGCATTAACAGGA | (SEQ ID NO:98) |
| AAAAAGTCTCACTTGTGATGCTGGGTCCAGCTTATAACCACACAATGGAAACCCCTGCCTC | |
| CTTCCTCCTTGTGGGTATCCCAGGACTGCAATCTTCACATCTTTGGCTGGCTATCTCACTGA | |
| GTGCCATGTACATCATAGCCCTGTTAGGAAACACCATCATCGTGACTGCAATCTGGATGGA | |
| TTCCACTCGGCATGAGCCCATGTATTGCTTTCTGTGTGTTCTGGCTGCTGTGGACATTGTTA | |
| TGGCCTCCTCGGTGGTACCCAGATGGTGAGCATCTTCTGCTCAGGAGACAGCTCAATCAG | |
| CTTTAGTGCTGTTTCACTCAGATGTTTTTTGTCCACTTAGCCACAGCTGTGGAGACGGGG | |
| CTGCTGCTGACCATGGCTTTTGACCGCTATGTAGCCATCTGCAAGCCTCTACACTACAAGA | |
| GAATTCTCACGCCTCAAGTGATGCTGGGAATGAGTATGGCCATCACCATCAGAGCTATCAT | |
| AGCCATAACTCCACTGAGTTGGATGGTGAGTCATCTACCTTTCTGTGGCTCCAATGTGGTT | |
| GTCCACTCCTACTGTGAGCACATAGCTTTGGCCAGGTTAGCATGTGCTGACCCCGTGCCCA | |
| GCAGTCTCTACAGTCTGATTGGTTCCTCTCTTATGGTGGGCTCTGATGTGGCCTTCATTGCT | |
| GCCTCCTATATCTTAATTCTCAAGGCAGTATTTGGTCTCTCCTCAAAGACTGCTCAGTTGAA | |
| AGCATTAAGCACATGTGGCTCCCATGTGGGGGTTATGGCTTTGTACTATCTACCTGGGATG | |
| GCATCCATCTATGCGGCCTGGTTGGGGCAGGATGTAGTGCCCTTGCACACCCAAGTCCTGC | |
| TAGCTGACCTGTACGTGATCATCCCAGCCACCTTAAATCCCATCATCTATGGCATGAGGAC | |
| CAAACAACTGCGGGAGAGAATATGGAGTTATCTGATGCATGTCCTCTTTGACCATTCCAAC | |
| CTGGGTTCATGA | |
| AOLFR52 sequences: | |
| MLGPAYNHTMETPASFLLVGIPGLQSSHLWLAISLSAMYITALLGNTLIVTAIWMDSTRHEPMY | (SEQ ID NO:99) |
| CFLCVLAAVDIVMASSVVPKMVSIFCSGDSSISFSACFTQMFFVHLATAVETGLLLTMAFDRYV | |
| AICKPLHYKRILTPQVMLGMSMAVTIRAVTFMTPLSWMMNHLPFVGSNVVVHSYCKHIALAR | |
| LACADPVPSSLYSLIGSSLMVGSDVAFIAASYILILRAVFDLSSKTAQLKALSTCGSHVGVMALY | |
| YLPGMASIYAAWLGQDIVPLHTQVLLADLYVIIPATLNPIIYGMRTKQLLEGIWSYLMHFLFDH | |
| SNLGS | |
| ATGCTGGGTCCAGCTTACAACCACACAATGGAAACCCCTGCCTCCTTCCTCCTTGTGGGTA | (SEQ ID NO:100) |
| TCCCAGGACTGCAATCTTCACATCTTTGGCTGGCTATCTCACTGAGTGCCATGTACATCAC | |
| AGCCCTGTAGGAAACACCCTCATCGTGACTGCAATCTGGATGGATTCCACTCGGCATGAG | |
| CCCATGTATTGCTTTCTGTGTGTCTGGCTGCTGTGGACATTGTTATGGCCTCCTCCGTGGT | |
| ACCCAAGATGGTGAGCATCTTCTGCTCGGGAGACAGCTCCATCAGCTTTAGTGCTTGTTTC | |
| ACTCAGATGTTTTTTGTCCACTTAGCCACAGCTGTGGAGACGGGGCTGCTGCTGACCATGG | |
| CTTTTGACCGCTATGTAGCCATCTGCAAGCCTCTACACTACAAGAGAATTCTCACGCCTCA | |
| AGTGATGCTGGGAATGAGTATGGCCGTCACCATCAGAGCTGTCACATTCATGACTCCACTG | |
| AGTTGGATGATGAATCATCTACCTTTCTGTGGCTCCAATGTGGTTGTCCACTCCTACTGTAA | |
| GCACATAGCTTTGGCCAGGTTAGCATGTGCTGACCCCGTGCCCAGCAGTCTCTACAGTCTG | |
| ATTGGTTCCTCTCTTATGGTGGGCTCTGATGTGGCCTCATTGCTGCCTCCTATATCTTAAT | |
| TCTCAGGGCAGTATTTGATCTCTCCTCAAAGACTGCTCAGTTGAAAGCATTAAGCACATGT | |
| GGCTCCCATGTGGGGGTTATGGCTTTGTACTATCTACCTGGGATGGCATCCATCTATGCGG | |
| CCTGGTTGGGGCAGGATATAGTGCCCTTGCACACCCAAGTGCTGCTAGCTGACCTGTACGT | |
| GATCATCCCAGCCACTTTAAATCCCATCATCTATGGCATGAGGACCAAACAATTGCTGGAG | |
| GGAATATGGAGTTATCTGATGCACTTCCTCTTTGACCACTCCAACCTGGGTTCATGA | |
| AOLFR54 sequences: | |
| MSDSNLSDNHLPDTFFLTGIPGLEAAHFWIAIPFCAMYLVALGNAALILVIAMDNALHAPMY | (SEQ ID NO:101) |
| LFLCLLSLTDLALSSTTVPKMLAILWLHAGEISFGGCLAQMFCVHSIYALESSILLAMAFDRYVA | |
| ICNPLRYTTILNHAVIGRIGFVGLFRSVAIVSPFIFLLRRLPYCGHRVMTHTYCEHMGIARLACA | |
| NITVNIVYGLTVALLAMGLDSILIAISYGFILHAVFHLPSHDAQHKALSTCGSHIGIILVFYIPAFF | |
| SFLTHRFGHHEVPKHVHIFLANLYVLVPPVLNPILGARTKEIRSRLLKLLHLGKTSI | |
| ATGTCAGATTCCAACCTCAGTGATAACCATCTTCCAGACACCTTCTTCTTAACAGGGATCC | (SEQ ID NO:102) |
| CAGGGCTGGAGGCTGCCCACTTCTGGATTGCCATCCCTTTCTGTGCCATGTATCTTGTAGC | |
| ACTGGTTGGAAATGCTGCCCTCATCCTGGTCATTGCCATGGACAATGCTCTTCATGCACCT | |
| ATGTACCTCTTCCTCTGCCTTCTCTCACTCACAGACCTGGCTCTCAGTTCTACCACTGTGCC | |
| CAAGATGCTGGCCATTTTGTGGCTCCATGCTGGTGAGATTTCCTTTGGTGGATGCCTGGCC | |
| CAGATGTTTTGTGTCCATTCTATCTATGCTCTGGAGTCCTCGATTCTACTGCCATGGCCTT | |
| TGATAGGTATGTGGCTATCTGTAACCCATTAAGGTATACAACCATTCTCAACCATGCTGTC | |
| ATAGGCAGAATTGGCTTTGTTGGGCTATTCCGTAGTGTGGCTATTGTCTCCCCCTTCATCTT | |
| CTTGCTGAGGCGACTCCCCTACTGTGGTCACCGTGTCATGACACACACATACTGTGAGCAT | |
| ATGGGCATCGCCCGACTGGCCTGTGCCAACATCACTGTCAATATTGTCTATGGGCTAACTG | |
| TGGCTCTGCTGGCCATGGGACTGGATTCCATTCTCATTGCCATTTCCTATGGCTTTATCCTC | |
| CATGCAGTCTTTCACCTTCCATCTCATGATGCCCAGCACAAAGCTCTGAGTACCTGTGGCT | |
| CCCACATTGGCATCATCCTGGTTTTCTACATCCCTGCCTTCTTCTCCTTCCTCACCCACCGC | |
| TTTGGTCACCACGAAGTCCCCAAGGATGTGCACATCTTTCTGGCTAATCTCTATGTGCTGG | |
| TGCCTCCTGTACTCAATCCTATTCTCTATGGAGCTAGAACCAAGGAGATTGCGGAGTCGACT | |
| TCTAAAACTGCTTCACCTGGGGAAGACTTCAATATGA | |
| AOLFR57 sequences: | |
| MSFQVTMFYLHWTMEKSNNSTLFILLLGFQNKNIEVLCFVLFLCYIAIWMGNLLIMISITCTQ | (SEQ ID NO:103) |
| LIHQPMYFFLNYSLSDLCYTSTVTPKLMVDLLAERKTISYNNCMIQLFTTHFFGGIEIFILTGM | |
| AYDRYVAICKPLHYTIIMSRQKCNTIIIVCCTGGFIHSASQFLLTIFVPFCGPNEIDHYFCDVYPLL | |
| KLACSNIHMIGLLVIANSGLIALVTFVVLLSYVFILYYTIRAYSAERRSKALATCSSHVIVVVLFF | |
| APALFIYIRPVTTFSEDKFALFYTIIPMFPLIYTLRNTEMKNAMRKVWCCQILLKRNQLF | |
| ATGCATTTCAGGTGACTTATATGTTCTATCTACACTGGACCATGGAAAAAAGCAATAATA | (SEQ ID NO:104) |
| GCACTTTGTTTATTCTCTTGGGGTTTTCCCAAAATAAGAACATTGAAGTCCTCTGCTTTGTA | |
| TTATTTTTGTTTTGCTACATTGCTATTTGGATGGGAAACTTACTCATAATGATTTCTATCAC | |
| GTGCACCCAGCTCATTCACCAACCCATGTATTTCTTCCTCAATTACCTCTCACTCTCCGACC | |
| TTTGCTACACATCCACAGTGACCCCCAAATTAATGGTTGACTTACTGGCAGAAAGAAAGAC | |
| CATTTCCTATAATAACTGTATGATACAACTCTTTACCACCCATTTTTTTGGAGGCATAGAGA | |
| TCTTCATTCTCACAGGGATGGCCTATGACCGCTATGTGGCCATTTGCAAGCCCCTGCACTA | |
| CACCATTATTATGAGCAGGCAAAAGTGTAACACAATCATCATAGTTTGTTGTACTGGGGGA | |
| TTTATACATTCTGCCAGTCAGTTTCTTCTCACCATCTTTGTACCATTTTGTGGCCCAAATGA | |
| GATAGATCACTACTTCTGTGATGTGTATCCTTTGCTGAAATTGGCCTGTTCTAATATACACA | |
| TGATAGGTCTCTTAGTCATTGCTAATTCAGGCTTAATTGCTTTGGTGACATTTGTTGTCTTG | |
| TTGTTGTCTTATGTTTTTATATTGTATACCATCAGAGCATACTCTGCAGAGAGACGCAGCA | |
| AAGCTCTTGCCACTTGTAGTTCTCATGTAATTGTTGTGGTCCTGTTTTTTGCTCCTGCATTG | |
| TTCATTTACATTAGACCGGTCACAACATTCTCAGAAGATAAAGTGTTTGCCCTTTTTTATAC | |
| CATCATTGCTCCCATGTTCAACCCTCTCATATACACGCTGAGAAACACAGAGATGAAGAAC | |
| GCCATGAGGAAAGTGTGGTGTTGTCAAATACTCCTGAAAAGAAATCAACTTTTCTGA | |
| AOLFR58 sequences: | |
| MFSMTTEALNNFALGCTNLLMTMIP9GIDLKQIFLCPNCRLYMIPVGAFIFSLGNMQNQSFVTEF | (SEQ ID NO:105) |
| VLLGLSQNPNVQEIVFVVFLFVYIATVGGNMLIVVTILSSPALLVSPMTTFLGFLSFLDACFSSVI | |
| TPKMIVDSLYVTKTISFEGCMMQLFAEHFFAGVEVIVLTAMAYDRYVAICKPLHYSSIMNRRL | |
| CGILMGVAWTGGLLHSMIQILFTFQLPFCGPNVINHFMCDLYPLLELACTDTHIFGLMVVINSG | |
| FICIINFSLLLVSYAVILLSLRTHSSEGRWKALSTCGSHIAVVILFFVPCIFVYTRPPSAFSLDKMA | |
| AIFYIILNPLLNPLIYTFRNKEVKQAMRRIWNRLMVVSDEKENIKL | |
| ATGTTCTCAATGACAACAGAAGCACTCAATAATTTTGCACTTGGATGTACCAACTTGTTAA | (SEQ ID NO:106) |
| TGACTATGATACCACAAATTGATCTGAAGCAAATTTTCCTTTGTCCTAATTGCAGACTATA | |
| CATGATCCCTGTTGGAGCTTTCATCTTTTCCTTGGGAAACATGCAAAACCAAAGCTTTGTA | |
| ACTGAGTTTGTCCTCCTGGGACTTTCACAGAATCCAAATGTTCAGGAAATAGTATTTGTTG | |
| TATTTTTGTTTGTCTACATTGCAACTGTTGGGGGCAACATGCTAATTGTAGTAACCATTCTC | |
| AGCAGCCCTGCTCTTCTGGTGTCTCCTATGTACTTCTTCTTGGGCTTCCTGTCCTTCCTGGA | |
| TGCGTGCTTCTCATCTGTCATCACCCCAAAGATGATTGTAGACTCCCTCTATGTGACAAAA | |
| ACCATCTCTTTTGAAGGCTGCATGATGCAGCTCTTTGCTGAACACTTCTTTGCTGGGGTGG | |
| AGGTGATTGTCCTCACAGCCATGGCCTATGATCGTTATGTGGCCATTTGCAAGCCCTTGCA | |
| TTACTCTTCTATCATGAACAGGAGGCTCTGTGGCATTCTGATGGGGGTAGCCTGGACAGGG | |
| GGCCTCTTGCATTCCATGATACAAATTCTTTTTACTTTCCAGCTTCCCTTTTGTGGCCCCAA | |
| TGTCATCAATCACTTTATGTGTGACTTGTACCCGTTACTGGAGCTTGCCTGCACTGATACTC | |
| ACATCTTTGGCCTCATGGTGGTCATCAACAGTGGGTTTATCTGCATCATAAACTTCTCCTTG | |
| TTGCTTGTCTCCTATGCTGTCATCTTGCTCTCTCTGAGAACACACAGTTCTGAAGGGCGCTG | |
| GAAAGCTCTCTCCACCTGTGGATCTCACATTGCTGTTGTGATTTTGTTCTTTGTCCCATGCA | |
| TATTTGTATATACACGACCTCCATCTGCTTTTTCCCTTGACAAAATGGCGGCAATATTTTAT | |
| ATCATCTTAAATCCCTTGCTCAATCCTTTGATTTACACTTTCAGGAATAAGGAAGTAAAAC | |
| AGGCCATGAGGAGAATATGGAACAGACTGATGGTGGTTTCTGATGAGAAAGAAAATATTA | |
| AACTTTAA | |
| AOLFR59 sequences: | |
| MGDWNNSDAVEPIFILRGFPGLEYVHSWLSILFCLAYLVAFMGNVTILSVIWIESSLHQPMYTTI | (SEQ ID NO:107) |
| SILAVNDLGMSLSTLPTMLAVLWLDAPEIQASACYAQLFFIHTFTFLESSVLLAMAFDRFVAICH | |
| PLHYPTILTNSVIGKIGLACLLRSLGVVLPTPLLLRHYHYCHGNALSHAFCLHQDVLRLSCTDA | |
| RTNSIYGLCVVIATLGVDSIFILLSYVLILNTVLDIASREEQLKALNTCVSHICVVLIFFVPVIGVS | |
| MVHRFGKHLSPIVHILMADIYLLLPPVLNPIVYSVRTKQIRLGILHKFVLRRRF | |
| ATGGGAGACTGGAATAACAGTGATGCTGTGGAGCCCATATTTATCCTGAGGGGTTTTFCCTG | (SEQ ID NO:108) |
| GACTGGAGTATGTTCATTCTTGGCTCTCCATCCTCTTCTGTCTTGCATATTTGGTAGCATTT | |
| ATGGGTAATGTTACCATCCTGTCTGTCATTTGGATAGAATCCTCTCTCCATCAGCCCATGTA | |
| TTACTTTATTTCCATCTTAGCAGTGAATGACCTGGGGATGTCCCTGTCTACACTTCCCACCA | |
| TGCTTGCTGTGTTATGGTTGGATGCTCCAGAGATCCAGGCAAGTGCTTGCTATGCTCAGCT | |
| GTTCTTCATCCACACATTCACATTCCTGGAGTCCTCAGTGTTGCTGGCCATGGCCTTTGACC | |
| GTTTTGTTGCTATCTGCCATCCACTGCACTACCCCACCATCCTCACCAACAGTGTAATTGGC | |
| AAAATTGGTTTGGCCTGTTTGCTACGAAGCTTGGGAGTTGTACTTCCCACACCTTTGCTACT | |
| GAGACACTATCACTACTGCCATGGCAATGCCCTCTCTCACGCCTTCTGTTTGCACCAGGAT | |
| GTTCTAAGATTATCCTGTACAGATGCCAGGACCAACAGTATTTATGGGCTTTGTGTAGTCA | |
| TTGCCACACTAGGTGTGGATTCAATCTTCATACTTCTTTCTTATGTTCTGATTCTTAATACT | |
| GTGCTGGATATTGCATCTCGTGAAGAGCAGCTAAAGGCACTCAACACATGTGTATCCCATA | |
| TCTGTGTGGTGCTTATCTTCTTTGTGCCAGTTATTGGGGTGTCAATGGTCCATCGCTTTGGG | |
| AAGCATCTGTCTCCCATAGTCCACATCCTCATGGCAGACATCTACCTTCTTCTTCCCCCAGT | |
| CCTTAACCCTATTGTCTATAGTGTCAGAACAAAGCAGATTCGTCTAGGAATTCTCCACAAG | |
| TTTGTCCTAAGGAGGAGGTTTTAA | |
| AOLFR60 sequences: | |
| MFLPNDTQFHPSSFLLLGIPGLETLHIWIGFPFCAVYMIALIGNFTILLVIKTDSSLHQPMFTTLA | (SEQ ID NO:109) |
| MLATTDVGLSTATIPKMLGIFWINLRGIIFEACLTQMFFIHNFTLMESAVLVAMAYDSYVAICN | |
| PLQYSAILTNKVVSVIGLGVFVRALIFVIPSILLILRLPFCGNHVIPHTYCEHMGLAHLSCASIKINI | |
| IYGLCAICNLVFDITVIALSYVHILCAVFRLPTHEPRLKSLSTCGSHVCVILAFYTPALFSFMTHC | |
| FGRNVPRYIHILLANLYVVVPPMLNPVIYGVRTKQIYKCVKKILLQEQGMEKEEYLIHTRF | |
| ATGTTCCTTCCCAATGACACCCAGTTTCACCCCTCCTCCTTCCTGTTGCTGGGGATCCCAGG | (SEQ ID NO:110) |
| ACTAGAAACACTTCACATCTGGATCGGCTTTCCCTTCTGTGCTGTGTACATGATCGCACTC | |
| ATAGGGAACTTCACTATTCTACTTGTGATCAAGACTGACAGCAGCCTACACCAGCCCATGT | |
| TCTACTTCCTGGCCATGTTGGCCACCACTGATGTGGGTCTCTCAACAGCTACCATCCCTAA | |
| GATGCTTGGAATCTTCTGGATCAACCTCAGAGGGATCATCTTTGAAGCCTGCCTCACCCAG | |
| ATGTTTTTTATCCACAACTTCACACTTATGGAGTCAGCAGTCCTTGTGGCAATGGCTTATG | |
| ACAGCTATGTGGCCATCTGCAATCCACTCCAATATAGCGCCATCCTCACCAACAAGGTTGT | |
| TTCTGTGATTGGTCTTGGTGTGTTTGTGAGGGCTTTAATTTTCGTCATTCCCTCTATACTTC | |
| TTATATTGCGGTTGCCCTTCTGTGGGAATCATGTAATTCCCCACACCTACTGTGAGCACAT | |
| GGGTCTTGCTCATCTATCTTGTGCCAGCATCAAAATCAATATTATTTATGGTTTATGTGCCA | |
| TTTGTAATCTGGTGTTTGACATCACAGTCATTGCCCTCTCTTATGTGCATATTCTTTGTGCT | |
| GTTTTCCGTCTTCCTACTCATGAGCCCCGACTCAAGTCCCTCAGCACATGTGGTTCACATGT | |
| GTGTGTAATCCTTGCCTTCTATACACCAGCCCTCTTTTCCTTTATGACTCATTGCTTTGGCC | |
| GAAATGTGCCCCGCTATATCCATATACTCCTAGCCAATCTCTATGTTGTGGTGCCACCAAT | |
| GCTCAATCCTGTCATATATGGAGTCAGAACCAAGCAGATCTATAAATGTGTAAAGAAAAT | |
| ATTATTGCAGGAACAAGGAATGGAAAAGGAAGAGTACCTAATACATACGAGGTTCTGA | |
| AOLFR61 sequences: | |
| MSIINTSYVEITTFFLVGMPGLEYAHIWISIPICSMYLIAILGNGTILFIITEPSLHGPMYTTLSML | (SEQ ID NO:111) |
| AMSDLGLSLSSLPTVLSIFLFNAPETSSSACFAQEFFIHGFSVLESSVLLIMSFDRFLAIHNPLRYT | |
| SILTTVRVAQIGIVFSFKSMLLVLPFPFTLRSLRYCKKNQLSHSYCLHQDVMKLACSDNRIDVIY | |
| GFFGALCLMVDFILIAVSYTLILKTVPGIASKKEELKALNTCVSHICAVIIFYLPIINLAVVHRFAG | |
| HVSPLINVLMANVLLLVPPLMKPIVYCVKTKQIRVRVVAKLCQWKI | |
| ATGTCCATTATCAACACATCATATGTTGAAATCACCACCTTCTTCTTGGTTGGGATGCCAG | (SEQ ID NO:112) |
| GGCTAGAATATGCACACATCTGGATCTCTATCCCCATCTGCAGCATGTATCTTATTGCTATT | |
| CTAGGAAATGGCACCATTCTTTTTATCATCAAGACAGAGCCCTCCTTGCATGGGCCCATGT | |
| ACTATTTTCTTTCCATGTTGGCTATGTCAGACTTGGGTTTGTCTTTATCATCTCTGCCCACT | |
| GTGTTAAGCATCTTCCTGTTCAATGCCCCTGAAACTTCTTCTAGTGCCTGCTTTGCCCAGGA | |
| ATTCTTCATTCATGGATTCTCAGTACTGGAGTCCTCAGTCCTCCTGATCATGTCATTTGATA | |
| GATTCCTAGCCATCCACAATCCTCTGAGATACACCTCAATCCTGACAACTGTCAGAGTTGC | |
| CCAAATAGGGATAGTATTCTCCTTTAAGAGCATGCTCCTGGTTCTTCCCTTCCCTTTCACTT | |
| TAAGAAGCTTGAGATATTGCAAGAAAAACCAATTATCCCATTCCTACTGTCTCCACCAGGA | |
| TGTCATGAAGTTGGCCTGTTCTGACAACAGAATTGATGTTATCTATGGCTTTTTTGGAGCA | |
| CTCTGCCTTATGGTAGACTTTATTCTCATTGCTGTGTCTTACACCCTGATCCTCAAGACTGT | |
| ACCGGGAATTGCATCCAAAAAGGAGGAGCTTAAGGCTCTCAATACTTGTGTTTCACACATC | |
| TGTGCAGTGATCATCTTCTACCTGCCCATCATCAACCTGGCCGTTGTCCACCGCTTTGCCGG | |
| GCATGTCTCTCCCCTCATTAATGTTCTCATGGCAAATGTTCTCCTACTTGTACCTCCGCTGA | |
| TGAAACCAATTGTTTATTGTGTAAAAACTAAACAGATTAGAGTGAGAGTTGTAGCAAAATT | |
| GTGTCAATGGAAGATTTAA | |
| AOLFR62 sequences: | |
| MFYHNKSIFHPVTFFLIGIPGLEDFHMWISGPFCSVYLVALLGNATILLVIKVEQTLREPMFTTL | (SEQ ID NO:113) |
| AILSTIDLALSATSVPRMLGIFWFDAHEINYGACVAQMFLIHAFTGMEAEVLLAMAFDRYVAIC | |
| APLHYATILTSLVLVGISMCIVIRPVLLTLPMVYLIYRLPFCQAHIIAHSYCEHMGIAKLSCGNIRI | |
| NGIYGLFVVSFFVLNLVLIGISYVYILRAVFRLPSHDAQLKALSTCGAHVGVICVFYIPSVFSFLT | |
| HRFGHQIPGYIHILVANLYLIIPPSLNPIIYGVRTKQIRERVLYVFTKK | |
| ATGTTTTATCACAACAAGAGCATATTTCACCCAGTCACATTTTTCCTCATTGGAATCCCAGG | (SEQ ID NO:114) |
| TCTGGAAGACTTCCACATGTGGATCTCCGGGCCTTTCTGCTCTGTTTACCTTGTGGCTTTGC | |
| TGGGCAATGCCACCATTCTGCTAGTCATCAAGGTAGAACAGACTCTCCGGGAGCCCATGTT | |
| CTACTTCCTGGCCATTCTTTCCACTATTGATTTGGCGCTTTCTGCAACCTCTGTGCCTCGCA | |
| TGCTGGGTATCTTCTGGTTTGATGCTCACGAGATTAACTATGGAGCTTGTGTGGCCCAGAT | |
| GTTTCTGATCCATGCCTTCACTGGCATGGAGGCTGAGGTCTTACTGGCTATGGCTTTTGAC | |
| CGTTATGTGGCCATCTGTGCTCCACTACATTACGCAACCATCTTGACATCCCTAGTGTTGGT | |
| GGGCATTAGCATGTGCATTGTAATTCGTCCCGTTTTACTTACACTTCCCATGGTCTATCTTA | |
| TCTACCGCCTACCCTTTTGTCAGGCTCACATAATAGCCCATTCCTACTGTGAGCACATGGG | |
| CATTGCAAAATTGTCCTGTGGAAACATTCGTATCAATGGTATCTATGGGCTTTTTGTAGTTT | |
| CTTTCTTTGTTCTGAACCTGGTGCTCATTGGCATCTCGTATGTTTACATTCTCCGTGCTGTC | |
| TTCCGCCTCCCATCACATGATGCTCAGCTAAAAGCCCTAAGCACGTGTGGCGCTCATGTTG | |
| GAGTCATCTGTGTTTTCTATATCCCTTCAGTCTTCTCTTTCCTTACTCATCGATTTGGACAC | |
| CAAATACCAGGTTACATTCACATTCTTGTTGCCAATCTCTATTTGATTATCCCACCCTCTCT | |
| CAACCCCATCATTTATGGGGTGAGGACCAAACAGATTCGAGAGCGAGTGCTCTATGTTTTT | |
| ACTAAAAAATAA | |
| AOLFR63 sequences: | |
| MSIINTSYVEITTFFLVGMPGLEYAHIWISIPICSMYLIAILGNGTILFIIKTEPSLHEPMYTTLSML | (SEQ ID NO:115) |
| AMSDLGLSLSSLPTVLSIFLFNAPEISSNACFAQEFFIHGFSVLESSVLLIMSFDRFLAIHNPLRYTS | |
| ILTTVRVAQIGIVFSFKSMLLVLPFPFTLRNLRYCKKNQLSHSYCLHQDVMKLACSDNRIDVIY | |
| GFFGALCLMVDFILIAVSYTLILKTVLGIASKKEQLKALNTCVSHICAVIIFYLPIINLAVVHRFAR | |
| HVSPLINVLMANVLLLVPPLTNPIVYCVKTKQIRVRVVAKLCQRKI | |
| ATGTCCATTATCAACACATCATATGTTGAAATCACCACCTTCTTCTTGGTTGGGATGCCAG | (SEQ ID NO:116) |
| GGCTAGAATATGCACACATCTGGATCTCTATCCCCATCTGCAGCATGTATCTTATTGCTATT | |
| CTAGGAAATGGCACCATTCTTTTTATCATCAAGACAGAGCCCTCCTTGCATGAGCCCATGT | |
| ACTATTTTCTTTCCATGTTGGCTATGTCAGACTTGGGTTTGTCTTTATCATCTCTGCCCACT | |
| GTGTTAAGCATCTTCCTGTTCAATGCTCCTGAAATTTCATCCAATGCCTGCTTTGCCCAGGA | |
| ATTCTTCATTCATGGATTCTCAGTACTGGAGTCCTCAGTCCTCCTGATCATGTCATTTGATA | |
| GATTCCTAGCCATCCACAACCCTCTGAGATACACCTCAATCCTGACAACTGTCAGAGTTGC | |
| CCAAATAGGGATAGTATTCTCCTTTAAGAGCATGCTCCTGGTTCTTCCCTTCCCTTTCACTT | |
| TAAGAAACTTGAGATATTGCAAGAAAAACCAATTATCCCATTCCTACTGTCTCCACCAGGA | |
| TGTCATGAAGTTGGCCTGTTCTGACAACAGAATTGATGTTATCTATGGCTTTTTTGGAGCA | |
| CTCTGCCTTATGGTAGACTTTATTCTCATTGCTGTGTCTTACACCCTGATCCTCAAGACTGT | |
| ACTGGGAATTGCATCCAAAAAGGAGCAGCTTAAGGCTCTCAATACTTGTGTTTCACACATC | |
| TGTGCAGTGATCATCTTCTACCTGCCCATCATCAACCTGGCCGTTGTCCACCGCTTTGCCCG | |
| GCATGTCTCTCCCCTCATTAATGTTCTCATGGCAAATGTTCTCCTACTTGTACCTCCACTGA | |
| CGAACCCAATTGTTTATTGTGTAAAAACTAAACAGATTAGAGTGAGAGTTGTAGCAAAATT | |
| GTGTCAACGGAAGATTTAA | |
| AOLFR64 sequences: | |
| MTILLNSSLQRATFFLTGFQGLEGLHGWISIPFCFIYLTVILGNLTILHVICTDATLHGPMYTTLG | (SEQ ID NO:117) |
| MLAVTDLGLCLSTLPTVLGIFWFDTREIGIPACFTQLFFIHTLSSMESSVLLSMSIDRSVAVCNPL | |
| HDSTVLTPACIVKMGLSSVLRSALLILPLPFLLKRFQYCHSHVLAHAYCLHLEIMKLACSSIIVN | |
| HIYGLFVVACTVGVDSLLIFLSYALILRTVLSIASHQERLRALNTCVSHICAVLLFYIPMIGLSLV | |
| HRFGEHLPRVVHLFMSYVYLLVPPLMNPIIYSIKTKQIRQRIIKKFQFIKSLRCFWKD | |
| ATGACAATTCTTCTTAATAGCAGCCTCCAAAGAGCCACTTTCTTCCTGACGGGCTTCCAAG | (SEQ ID NO:118) |
| GTCTAGAAGGTCTCCATGGCTGGATCTCTATTCCCTTCTGCTTCATCTACCTGACAGTTATC | |
| TTGGGGAACCTCACCATTCTCCACGTCATTTGTACTGATGCCACTCTCCATGGACCCATGT | |
| ACTATTTCTTGGGCATGCTAGCTGTCACAGACTTAGGCCTTTGCCTTTCCACACTGCCCACT | |
| GTGCTGGGCATTTTCTGGTTTGATACCAGAGAGATTGGCATCCCTGCCTGTTTCACTCAGC | |
| TCTTCTTCATCCACACCTTGTCTTCAATGGAGTCATCAGTTCTGTTATCCATGTCCATTGAC | |
| CGCTCCGTGGCCGTCTGCAACCCACTGCATGACTCCACCGTCCTGACACCTGCATGTATTG | |
| TCAAGATGGGGCTAAGCTCAGTGCTTAGAAGTGCTCTCCTCATCCTCCCCTTGCCATTCCTC | |
| CTGAAGCGCTTCCAATACTGCCACTCCCATGTGCTGGCTCATGCTTATTGTCTTCACCTGGA | |
| GATCATGAAGCTGGCCTGCTCTAGCATCATTGTCAATCACATCTATGGGCTCTTTGTTGTG | |
| GCCTGCACCGTGGGTGTGGACTCCCTGCTCATCTTTCTCTCATACGCCCTCATCCTTCGCAC | |
| CGTGCTCAGCATTGCCTCCCACCAGGAGCGACTCCGAGCCCTCAACACCTGTGTCTCTCAT | |
| ATCTGTGCTGTACTGCTCTTCTACATCCCCATGATTGGCTTGTCTCTTGTGCATCGCTTTGG | |
| TGAACATCTGCCCCGCGTTGTACACCTCTTCATGTCCTATGTGTATCTGCTGGTACCACCCC | |
| TTATGAACCCCATCATCTACAGCATCAAGACCAAGCAAATTCGCCAGCGCATCATTAAGAA | |
| GTTTCAGTTTATAAAGTCACTTAGGTGTTTTTGGAAGGATTAA | |
| AOLFR65 sequences: | |
| MAGRMSTSNHTQFHPSSFLLLGIPGLEDVHIWIGVPFFFVYLVALLGNTALLFVIQTEQSLHEPM | (SEQ ID NO:119) |
| YTTLAMLDSIDLGLSTATIPKMLGIFWFNTKEISFGGCLSHMFFIHFFTAMESIVLVAMAFDRYI | |
| AICKPLRYTMILTSKIISLIAGIAVLRSLYMVVPLVFLLLRLPFCGHRIIPHTYCEHMGIARLACAS | |
| IKVNIRFGLGNISLLLLDVILIILSYVRILYAVFCLPSWEARLKALNTCGSHIGVILAFFTPAFFSFL | |
| THRFGHNIPQYIHIILANLYVVVPPALNPVIYGVRTKQIRERVLRIFLKTNH | |
| ATGGCAGGAAGAATGTCTACGTCTAATCACACCCAGTTCCATCCTTCTTCATTCCTACTGCT | (SEQ ID NO:120) |
| GGGTATCCCAGGGCTAGAAGATGTGCACATTTGGATTGGAGTCCCTTTTTTCTTTGTGTAT | |
| CTTGTTGCACTCCTGGGAAACACTGCTCTCTTGTTTGTGATCCAGACTGAGCAGAGTCTCC | |
| ATGAGCCTATGTACTACTTCCTGGCCATGTTGGATTCCATTGACCTGGGCTTGTCTACAGC | |
| CACCATCCCCAAAATGTTGGGCATCATCTTCTGGTTCAATACCAAAGAAATATCTTTTGGAGGC | |
| TGCCTTTCTCACATGTTCTTCATCCATTTCTTCACTGCTATGGAGAGCATTGTGTTGGTGGC | |
| CATGGCCTTTGACCGCTACATTGCCATTTGCAAACCTCTTCGGTACACCATGATCCTCACCA | |
| GCAAAATCATCAGCCTCATTGCAGGCATTGCTGTCCTGAGGAGCCTGTACATGGTTGTTCC | |
| ACTGGTGTTTCTCCTTCTGAGGCTGCCCTTCTGTGGGCATCGTATCATCCCTCATACTTATT | |
| GTGAGCACATGGGCATTGCCCGTCTGGCCTGTGCCAGCATCAAAGTCAACATTAGGTTTGG | |
| CCTTGGCAACATATCTCTCTTGTTACTGGATGTTATCCTTATTATTCTCTCCTATGTCAGGA | |
| TCCTGTATGCTGTCTTCTGCCTGCCCTCCTGGGAAGCTCGACTCAAAGCTCTCAACACCTGT | |
| GGTTCTCATATTGGTGTTATCTTAGCCTTTTTTACACCAGCATTTTTTTCATTCTTGACACA | |
| TCGTTTTGGCCATAATATCCCACAGTATATACATATTATATTAGCCAACCTGTATGTGGTTG | |
| TCCCACCAGCCCTCAATCCTGTAATCTATGGAGTCAGGACAAAGCAGATTCGAGAGAGAG | |
| TGCTGAGGATTTTTCTCAAGACCAATCACTAA | |
| AOLFR66 sequences: | |
| MSFLNGTSLTPASFILNGIPGLEDVHLWISFPLCTMYSIAITGNFGLMYLIYCDEALHRPMYVFL | (SEQ ID NO:121) |
| ALLSFTDVLMCTSTLPNTLFILWFNLKEIDFKACLAQMFFVHTFTGMESGVLMLMALDHCVAI | |
| CFPLRYATILTNSVIAKAGFLTFLRGVMLVIPSTFLTKRLPYCKGNVIPHTYCDHMSVAKISCGN | |
| VRVNAIYGLIVALLIGGFDILCITISYTMILQAVVSLSSADARQKAFSTCTAHFCAIVLTYVPAFF | |
| TFFTHHFGGHTIPLHIHIIMANLYLLMPPTMNPIVYGVKTRQVRESVIRFFLKGKDNSHNF | |
| ATGTCATTTCTAAATGGCACCAGCCTAACTCCAGCTTCATTCATCCTAAATGGCATCCCTG | (SEQ ID NO:122) |
| GTTTGGAAGATGTGCATTTGTGGATCTCCTTCCCACTGTGTACCATGTACAGCATTGCTATT | |
| ACAGGGAACTTCGGCCTTATGTACCTCATCTACTGTGATGAGGCCTTACACAGACCTATGT | |
| ATGTCTTCCTTGCCCTTCTTTCCTTCACAGATGTGCTCATGTGCACCAGCACCCTTCCCAAC | |
| ACTCTCTTCATATTGTGGTTTAATCTCAAGGAGATTGATTTTAAAGCCTGCCTCGCGCAGAT | |
| GTTCTTTGTGCACACCTTCACAGGGATGGAGTCTGGGGTGCTCATGCTCATGGCCCTGGAC | |
| CACTGTGTGGCCATCTGCTTCCCTCTGCGTTATGCCACCATCCTCACTAATTCAGTCATTGC | |
| TAAAGCTGGGTTCCTCACTTTTCTTAGGGGTGTGATGCTTGTTATCCCTTCCACTTCCTCA | |
| CCAAGCGCCTTCCATACTGCAAGGGCAACGTCATACCCCACACCTACTGTGACCACATGTC | |
| TGTGGCCAAGATATCTTGTGGTAATGTCAGGGTTAACGCCATCTATGGTTTGATAGTTGCC | |
| CTGCTGATTGGGGGCTTTGATATCCTGTGCATTACAATCTCCTACACTATGATTCTTCAAGC | |
| AGTTGTGAGTCTATCATCAGCAGATGCTCGACAGAAGGCCTTCAGCACCTGCACTGCCCAC | |
| TTCTGTGCCATAGTCCTCACCTATGTTCCAGCCTTCTTTACCTTCTTTACACACCATTTTGG | |
| GGGACACACCATTCCTCTACACATACATATTATTATGGCTAATCTCTACCTACTAATGCCTC | |
| CCACAATGAACCCTATTGTGTATGGGGTGAAAACCAGGCAGGTACGAGAAAGTGTCATTA | |
| GGTTCTTTCTTAAGGGAAAGGACAATTCTCATAACTTTTAA | |
| AOLFR67 sequences: | |
| MSGDNSSSLTPGFFILNGVPGLEATHIWISLPFCFMYIIAVVGNCGLICLISHEEALHRPMYTTLA | (SEQ ID NO:123) |
| LLSFTDVTLCTTMVPNMLCIFWFNLKEIDFNACLAQMFFVHMLTGMESGVLMLMALDRYVAI | |
| CYPLRYATILTNPVIAKAGLATFLRINVMLIIPFTLLTKRLPYCRGNFIPHTYCDHMSVAKVSCGN | |
| FKVNAIYGLMVALLIGVFDICCISVSYTMILQAVMSLSSADARHKAFSTCTSHMCSIVITYVAAF | |
| FTFFTHRFVGHNIPNHIHIIVANLYLLLPPTMNPIVYGVKTKQIQEGVIKFLLGDKVSFTYDK | |
| ATGTCTGGGGACAACAGCTCCAGCCTGACCCCAGGATTCTTTATCTTGAATGGCGTTCCTG | (SEQ ID NO:124) |
| GGCTGGAAGCCACACACATCTGGATCTCCCTGCCATTCTGCTTTATGTACATCATTGCTGTC | |
| GTGGGGAACTGTGGGCTCATCTGCCTCATCAGCCATGAGGAGGCCCTGCACCGGCCCATGT | |
| ACTACTTCCTGGCCCTGCTCTCCTTCACTGATGTCACCTTGTGCACCACCATGGTACCTAAT | |
| ATGCTGTGCATATTCTGGTTCAACCTCAAGGAGATTGACTTTAACGCCTGCCTGGCCCAGA | |
| TGTTTTTTGTCCATATGCTGACAGGGATGGAGTCTGGGGTGCTCATGCTCATGGCCCTGGA | |
| CCGCTATGTGGCCATCTGCTACCCCTTACGCTATGCCACCATCCTTACCAACCCTGTCATCG | |
| CCAAGGCTGGTCTTGCCACCTTCTTGAGGAATGTGATGCTCATCATCCCATTCACTCTCCTC | |
| ACCAAGCGCCTGCCCTATTGCCGGGGGAACTTCATCCCCCACACCTACTGTGACCATATGT | |
| CTGTGGCCAAGGTATCCTGTGGCAATTTCAAGGTCAATGCTATTTATGGTCTGATGGTTGC | |
| TCTCCTGATTGGTGTGTTTGATATCTGCTGTATCTCTGTATCTTACACTATGATTTTGCAGG | |
| CTGTTATGAGCCTGTCATCAGCAGATGCTCGTCACAAAGCCTTCAGCACCTGCACATCTCA | |
| CATGTGTTCCATTGTGATCACCTATGTTGCTGCTTTTTTCACTTTTTTCACTCATCGTTTTGT | |
| AGGACACAATATCCCAAACCACATACACATCATCGTGGCCAACCTTTATCTGCTACTGCCT | |
| CCTACCATGAACCCAATTGTTTATGGAGTCAAGACCAAGCAGATTCAGGAAGGTGTAATTA | |
| AATTTTTACTTGGAGACAAGGTTAGTTTTACCTATGACAAATGA | |
| AOLFR68 sequences: | |
| MTTHRNDTLSTEASDFLLNCFVRSPSWQHWLSLPLSLLFLLAVGANTTLLMTIWLEASLHQPL | (SEQ ID NO:125) |
| YYLLSLLSLLDIVLCLTVIPKVLTIFWFDLRPISFPACFLQMYIMNCFLAMESCTFMVMAYDRY | |
| VAICHPLRYPSIITDHFVVKAAMFILTRNVLMTLPIPILSAQLRYCGRNVIENCICANMSVSRLSC | |
| DDVTINHLYQFAGGWTLLGSDLILIFLSYTFILRAVLRLKAEGAVAKALSTCGSHFMLILFFSTIL | |
| LVFVLTHVAKKKVSPDVPVLLNVLHHVIPAALNPIIYGVRTQEIKQGMQRLLKKGC | |
| ATGACAACACACCGAAATGACACCCTCTCCACTGAAGCTTCAGACTTCCTCTTGAATTGTT (SEQ ID NO:126) | |
| TTGTCAGATCCCCCAGCTGGCAGCACTGGCTGTCCCTGCCCCTCAGCCTCCTTTTCCTCTTG | |
| GCCGTAGGGGCCAACACCACCCTCCTGATGACCATCTGGCTGGAGGCCTCTCTGCACCAGC | |
| CCCTGTACTACCTGCTCAGCCTCCTCTCCCTGCTGGACATCGTGCTCTGCCTCACTGTCATC | |
| CCCAAGGTCCTGACCATCTTCTGGTTTGACCTCAGGCCCATCAGCTTCCCTGCCTGCTTCCT | |
| CCAGATGTACATCATGAATTGTTTCCTAGCCATGGAGTCTTGCACATTCATGGTCATGGCC | |
| TATGATCGTTATGTAGCCATCTGCCACCCACTGAGATATCCATCAATCATCACTGATCACTT | |
| TGTAGTCAAGGCTGCCATGTTTATTTTGACCAGAAATGTGCTTATGACTCTGCCCATCCCC | |
| ATCCTTTCAGCACAACTCCGTTATTGTGGAAGAAATGTCATTGAGAACTGCATCTGTGCCA | |
| ATATGTCTGTTTCCAGACTCTCCTGCGATGATGTCACCATCAATCACCTTTACCAATTTGCT | |
| GGAGGCTGGACTCTGCTAGGATCTGACCTCATCCTTATCTTCCTCTCCTACACCTTCATTCT | |
| GCGAGCTGTGCTGAGACTCAAGGCAGAGGGTGCCGTGGCAAAGGCCCTAAGCACATGTGG | |
| CTCCCACTTCATGCTCATCCTCTTCTTCAGCACCATCCTTCTGGTTTTTGTCCTCACACATGT | |
| GGCTAAGAAGAAAGTCTCCCCTGATGTGCCAGTCTTGCTCAATGTTCTCCACCATGTCATT | |
| CCTGCAGCCCTTAACCCCATCATTTACGGGGTGAGAACCCAAGAAATTAAGCAGGGAATG | |
| CAGAGGTTGTTGAAGAAAGGGTGCTAA | |
| AOLFR69 sequences: | |
| MSYSIYKSTVNIPLSHGVVHSFCHNMNCNFMHIFKFVLDFNMKNVTEVTLFVLKGFTDNLELQ (SEQ ID NO:127) | |
| TIFFFLFLAIYLFTLMGNLGLILVVIRDSQLHKPMYTTLSMLSSVDACYSSVITPNMLVDFTTKN | |
| KVISFLGCVAQVFLACSFGTTECFLLAAMAYDRYVAIYNPLLYSVSMSPRVYMPLINASYVAGI | |
| LHATIHTVATFSLSFCGANEIRRVFCDIPPLLAISYSDTHTNQLLLFTTVGSIELVTILIVLISYGLIL | |
| LAILKMYSAEGRRKVFSTCGAHLTGVSIYYGTILFMYVRPSSSYASDHDMIVSIFYTIVIPLLNPV | |
| IYSLRNKDVKDSMKKMFGKNQVINKVTTHTKK | |
| ATGTCGTACAGTATATACAAGAGCACAGTTAACATCCCCTTGAGTCATGGTGTTGTTCATT (SEQ ID NO:128) | |
| CTTTTTGTCATAATATGAACTGTAACTTTATGCATATCTTCAAGTTTGTTCTAGATTTCAAC | |
| ATGAAGAATGTCACTGAAGTTACCTTATTTGTACTGAAGGGCTTCACAGACAATCTTGAAC | |
| TGCAGACTATCTTCTTCTTCCTGTTTCTAGCAATCTACCTCTTCACTCTCATGGGAAATTTA | |
| GGACTGATTTTAGTGGTCATTAGGGATTCCCAGCTCCACAAACCCATGTACTATTTTCTGA | |
| GTATGTTGTCTTCTGTGGATGCCTGCTATTCCTCAGTTATTACCCCAAATATGTTAGTAGAT | |
| TTTACGACAAAGAATAAAGTCATTTCATTCCTTGGATGTGTAGCACAGGTGTTTCTTGCTT | |
| GTAGTTTTGGAACCACAGAATGCTTTCTCTTGGCTGCAATGGCTTATGATCGCTATGTAGC | |
| CATCTACAACCCTCTCCTGTATTCAGTGAGCATGTCACCCAGAGTCTACATGCCACTCATC | |
| AATGCTTCCTATGTTGCTGGCATTTTACATGCTACTATACATACAGTGGCTACATTTAGCCT | |
| ATCCTTCTGTGGAGCCAATGAAATTAGGCGTGTCTTTTGTGATATCCCTCCTCTCCTTGCTA | |
| TTTCTTATTCTGACACTCACACAAACCAGCTTCTACTCTTCTACTTTGTGGGCTCTATCGAG | |
| CTGGTCACTATCCTGATTGTTCTGATCTCCTATGGTTTGATTCTGTTGGCCATTCTGAAGAT | |
| GTATTCTGCTGAAGGGAGGAGAAAAGTCTTCTCCACATGTGGAGCTCACCTAACTGGAGT | |
| GTCAATTTATTATGGGACAATCCTCTTCATGTATGTGAGACCAAGTTCCAGCTATGCTTCG | |
| GACCATGACATGATAGTGTCAATATTTTACACCATTGTGATTCCCTTGCTGAATCCCGTCAT | |
| CTACAGTTTGAGGAACAAAGATGTAAAAGACTCAATGAAAAAAATGTTTGGGAAAAATCA | |
| GGTTATCAATAAAGTATATTTTCATACTAAAAAATAA | |
| AOLFR70 sequences: | |
| MDSTFTGYNLYNLQVKTEMDKLSSGLDIYRNPLKNKTEVTMFILTGFTDDFELQVFLFLLFFAI (SEQ ID NO:129) | |
| YLFTLlGNLGLVVLVIEDSWLHNPMYTTLSVLSFLDACYSTVVTPKMLVNFLAKNKSISFIGCA | |
| TQMLLFVTFGTTECFLLAAMAYDHYVAIYNPLLYSVSMSPRVYVPLITASYVAGILHATIHIVA | |
| TFSLSFCGSNEIRHVFCDMPPLLAISCSDTHTNQLLLFTTVGSIEIVTILIVLISCDFILLSILKMHSA | |
| KGRQKAFSTCGSHLTGVTIYHGTILVSYMRPSSSYASDHDIIVSIFYTIVIPKLNPIIYSLRNKEVK | |
| KAVKKMLKLVYK | |
| ATGGACTCCACTTTCACAGGCTATAACCTTTATAACCTGCAAGTAAAAACTGAAATGGACA (SEQ ID NO:130) | |
| AGTTGTCATCAGGTTTGGATATATACAGGAATCCACTGAAGAACAAGACTGAAGTCACCA | |
| TGTTTATATTGACAGGCTTCACAGATGATTTTGAGCTGCAAGTCTTCCTATTTTTACTATTT | |
| TTTGCAATCTATCTCTTTACCTTGATAGGCAATTTAGGGCTGGTTGTGTTGGTCATTGAGG | |
| ATTCCTGGCTCCACAACCCCATGTATTATTTTCTTAGTGTTTTATCATTCTTGGATGCTTGC | |
| TATTCTACAGTTGTCACTCCAAAAATGTTGGTCAATTTCCTGGCAAAAAATAAATCCATTT | |
| CATTTATCGGATGTGCAACACAGATGCTTCTTTTTGTTACTTTTGGAACTACAGAATGTTTT | |
| CTCTTGGCTGCAATGGCTTATGATCACTATGTAGCCATCTACAACCCTCTCCTGTATTCAGT | |
| GAGCATGTCACCCAGAGTCTATGTGCCACTCATCACTGCTTCCTACGTTGCTGGCATTTTAC | |
| ATGCTACTATACATATAGTGGCTACATTTAGCCTGTCCTTCTGTGGATCCAATGAAATTAG | |
| GCATGTCTTTTGTGATATGCCTCCTCTCCTTGCTATTTCTTGTTCTGACACTCACACAAACC | |
| AGCTTCTACTCTTCTACTTTGTGGGTTCTATTGAGATAGTCACTATCCTGATTGTCCTCATT | |
| TCCTGTGATTTCATTCTGTTGTCCATTCTGAAGATGCATTCTGCTAAGGGAAGGCAAAAGG | |
| CCTTCTCTACATGTGGCTCTCACCTAACTGGAGTGACAATTTATCATGGAACAATTCTCGTC | |
| AGTTATATGAGACCAAGTTCCAGCTATGCTTCAGACCATGACATCATAGTGTCAATATTTT | |
| ACACAATTGTGATTCCCAAGTTGAATCCCATCATCTATAGTTTGAGGAACAAAGAAGTAAA | |
| AAAGGCAGTGAAGAAAATGTTGAAATTGGTTTACAAATGA | |
| AOLFR71 sequences: | |
| MGRRNNTNVPDFILTGLSDSEEVQMALFILFLLIYLITMLGNVGMILIIRLDLQLHTPMTTFLTH (SEQ ID NO:131) | |
| LSFIDLSYSTVITPKTLANLLTSNYISFMGCFAQMFFFVFLGAAECFLLSSMAYDRYVAICSPLRY | |
| PVIMSKRLCCALVTGPYVISFINSFVNVVWMSRLHFCDSNVVRHFFCDTSPILALSCMDTYDIEI | |
| MIHILAGSTLMVSLITISASYVSILSTILKINSTSGKQKALSTCASHLLGVTIFYGTMIFTYLKPRK | |
| SYSLGRDQVASVFYTIVIPMLNPLIYSLRNKEVKNALIRVMQRRQDSR | |
| ATGGGTAGAAGAAATAACACAAATGTGCCTGACTTCATCCTTACGGGACTGTCAGATTCTG (SEQ ID NO:132) | |
| AAGAGGTCCAGATGGCCCTCTTTATACTATTTCTCCTGATATACCTAATTACTATGCTGGGC | |
| AATGTGGGGATGATATTGATAATCCGCCTGGACCTCCAGCTTCACACTCCCATGTATTTTT | |
| TCCTTACTCACTTGTCATTTATTGACCTCAGTTACTCAACTGTCATCACACCTAAAACCTTA | |
| GCGAACTTACTGACTTCCAACTATATTTCCTTCATGGGCTGCTTTGCCCAGATGTTCTTTTT | |
| TGTCTTCTTGGGAGCTGCTGAATGTTTTCTTCTCTCATCAATGGCCTATGATCGCTACGTAG | |
| CTATCTGCAGTCCTCTACGTTACCCAGTTATTATGTCCAAAAGGCTGTGTTGCGCTCTTGTC | |
| ACTGGGCCCTATGTGATTAGCTTTATCAACTCCTTTGTCAATGTGGTTTGGATGAGCAGAC | |
| TGCATTTCTGCGACTCAAATGTAGTTCGTCACTTTTTCTGCGACACGTCTCCAATTTTAGCT | |
| CTGTCCTGCATGGACACATACGACATTGAAATCATGATACACATTTTAGCTGGTTCCACCC | |
| TGATGGTGTCCCTTATCACAATATCTGCATCCTATGTGTCCATTCTCTCTACCATCCTGAAA | |
| ATTAATTCCACTTCAGGAAAGCAGAAAGCTTTGTCTACTTGTGCCTCTCATCTCTTGGGAG | |
| TCACCATCTTTTATGGAACTATGATTTTTACTTATTTAAAACCAAGAAAGTCTTATTCTTTG | |
| GGAAGGGATCAAGTGGCTTCTGTTTTTTATACTATTGTGATTCCCATGCTGAATCCACTCAT | |
| TTATAGTCTTAGAAACAAAGAAGTTAAAAATGCTCTCATTAGAGTCATGCAGAGAAGACA | |
| GGACTCCAGGTAA | |
| AOLFR72 sequences: | |
| MAPENFTRVTEFILTGVSSCPELQIPLFLVFLVLYGLTMAGNLGIITLTSVDSRLQTPMTTFLQHL (SEQ ID NO:133) | |
| ALINLGNSTVIAPKMLINFLVKKKTTSFYECATQLGGFLFFIVSEVIMLALMACDRYVAICNPLL | |
| YMVVVSRRLCLLLVSLTYLYGFSTAIVVSSYVFSVSYCSSNIINHFYCDNVPLLALSCSDTYLPE | |
| TVVFISAATNVVGSLIIVLVSTTNIVLSILKICSSEGRKKAFSTCASHMMAVTIFYGTLLFMYVQP | |
| RSNHSLDTDDKMASVFYTLVIPMLNPLIYSLRNKDVKTALQRFMTNLCYSFKTM | |
| ATGGCTCCTGAAAATTTCACCAGGGTCACTGAGTTTATTCTTACAGGTGTCTCTAGCTGTC | (SEQ ID NO:134) |
| CAGAGCTCCAGATTCCCCTCTTCCTGGTCTTTCTGGTGCTCTATGGGCTGACCATGGCAGG | |
| GAACCTGGGCATCATCACCCTCACCAGTGTTGACTCTCGACTTCAAACCCCCATGTACTTTT | |
| TCCTGCAACATCTGGCTCTCATTAATCTTGGTAACTCTACTGTCATTGCCCCTAAAATGCTG | |
| ATTAACTTTTTAGTAAAGAAGAAAACTACCTCATTCTATGAATGTGCCACCCAACTGGGAG | |
| GGTTCTTGTTCTTTATTGTATCGGAGGTAATCATGCTGGCTTTGATGGCCTGTGACCGCTAT | |
| GTGGCTATTTGTAACCCTCTGCTGTACATGGTGGTGGTGTCTCGGCGGCTCTGCCTCCTGCT | |
| GGTCTCCCTCACATACCTCTATGGCTTTTCTACAGCTATTGTGGTTTCATCTTATGTATTCT | |
| CTGTGTCTTATTGCTCTTCTAATATAATCAATCATTTTTACTGTGATAATGTTCCTCTGTTA | |
| GCATTATCTTGCTCTGATACTTACTTACCAGAAACAGTTGTCTTTATATCTGCAGCAACAA | |
| ATGTGGTTGGTTCCTTGATTATAGTTCTAGTATCTTATTTCAATATTGTTTTGTCTATTTTA | |
| AAAATATGTTCATCAGAAGGAAGGAAAAAAGCCTTTTCTACCTGTGCTTCACATATGATGG | |
| CAGTCACAATTTTTTATGGGACATTGCTATTCATGTATGTGCAGCCCCGAAGTAACCATTC | |
| ATTGGATACTGATGATAAGATGGCTTCTGTGTTTTACACGTTGGTAATTCCTATGCTGAAT | |
| CCCTTGATCTACAGCCTGAGGAATAAGGATGTGAAGACTGCTCTACAGAGATTCATGACA | |
| AATCTGTGCTATTCCTTTAAAACAATGTAA | |
| AOLFR73 sequences: | |
| MNHVVKHNHTAVTKVTEFILMGITDNPGLQAPLFGLFLIIYLVTVIGNLGMVILTYLDSKLHTP | (SEQ ID NO:135) |
| MTTFLRHLSITDLGYSTVIAPKMLVNFIVHKNTISYNWYATQLAFFEIFIISELFILSAMAYDRYV | |
| AICKPLLYVIIMAEKVLWVLVIVPYLYSTFVSLFLTIKLFKLSFCGSNIISTTYCDCIPLMSILCSDT | |
| NELELIILIFSGCNLLFSLSIVLISYMFILVAILRMNSRKGRYKAFSTCSSHLTVVIMFYGTLLFIYL | |
| QPKSSHTLAIDKMASVFYTLLIPMLNPLIYSLRNKEVKDALKRTLTNRFKIPI | |
| ATGAATCATGTGGTAAAACACAATCACACGGCAGTGACCAAGGTGACTGAATTTATTCTCA | (SEQ ID NO:136) |
| TGGGGATTACAGACAACCCTGGGCTGCAGGCTCCACTGTTTGGACTCTTCCTCATCATATA | |
| TCTGGTCACAGTGATAGGCAATCTGGGCATGGTTATCTTGACCTACTTGGACTCCAAGCTA | |
| CACACCCCCATGTACTTTTTCCTTAGACATTTGTCAATCACTGATCTTGGTTACTCCACTGT | |
| CATTGCCCCGAAGATGTTAGTAAACTTCATAGTGCACAAAAACACAATTTCTTACAATTGG | |
| TATGCCACTCAGCTAGCATTCTTTGAGATTTTCATCATCTCTGAGCTCTTTATTCTATCAGC | |
| AATGGCCTATGATCGCTACGTAGCCATGTGTAAACCTCTTCTGTACGTGATCATCATGGCA | |
| GAGAAAGTACTTTGGGTGCTGGTAATTGTTCCCTATCTCTATAGCACGTTTGTGTCACTATT | |
| TCTCACAATTAAGTTATTTAAACTGTCCTTCTGTGGCTCAAACATAATCAGCTATTTTTACT | |
| GTGACTGTATCCCTCTGATGTCCATACTCTGTTCTGACACAAATGAATTAGAATTAATAAT | |
| TTTGATCTTCTCAGGCTGTAATTTGCTCTTCTCCCTCTCAATTGTTCTCATATCCTACATGTT | |
| TATTCTAGTGGCCATTCTCAGAATGAACTCAAGGAAAGGGAGGTACAAAGCCTTCTCCACC | |
| TGTAGCTCTCATCTGACAGTGGTGATCATGTTCTATGGGACATTGTTATTTATTTACTTGCA | |
| ACCCAAGTCCAGTCATACTTTGGCTATTGATAAAATGGCCTCAGTGTTTTATACCCTGTTG | |
| ATTCCTATGCTGAATCCGTTGATCTACAGCCTAAGGAACAAAGAAGTAAAAGATGCTCTAA | |
| AGAGAACTTTAACCAATCGATTCAAAATTCCCATTTAA | |
| AOLFR74 sequences: | |
| MEQHNLTTVNEFILTGITDIAELQAPLFALFLMIYVISVMGNLGMIVLTKLDSRLQTPMTTFLRH | (SEQ ID NO:137) |
| LAFMDLGYSTTVGPKMLVNFVVDKNIISYTTCATQLAFFLVFIGSELFILSAMSYDLYVAICNPL | |
| LYTVIMSRRVCQVLVAIPYLYCTFISLLVTIKIFTLSFCGYNVISHFYCDSLPLLPLLCSNTHEIELI | |
| ILIFAAIDLISSLLIVLLSYLLILVAILRMNSAGRQKAFSTCGAHLTVVIVFYGTLLFMYVQPKSSH | |
| SFDTDKVASIFYTLVIPMLNPLIYSLRNKDVKYALRRTWNNLCNIFV | |
| ATGGAACAACACAATCTAACAACGGTGAATGAATTCATTCTTACGGGAATCACAGATATC | (SEQ ID NO:138) |
| GCTGAGCTGCAGGCACCATTATTTGCATTGTTCCTCATGATCTATGTGATCTCAGTGATGG | |
| GCAATTTGGGCATGATTGTCCTCACCAAGTTGGACTCCAGGTTGCAAACCCCTATGTACTT | |
| TTTTCTCAGACATCTGGCTTTCATGGATCTTGGTTATTCAACAACTGTGGGACCCAAAATG | |
| TTAGTAAATTTTGTTGTGGATAAGAATATAATTTCTTATTATTTTTGTGCAACACAGCTAGC | |
| TTTCTTTCTTGTGTTCATTGGTAGTGAACTTTTTATTCTCTCAGCCATGTCCTACGACCTCT | |
| ATGTGGCCATCTGTAACCCTCTGCTATACACAGTAATCATGTCACGAAGGGTATGTCAGGT | |
| GCTGGTAGCAATCCCTTACCTCTATTGCACATTCATTTCTCTTCTAGTCACCATAAAGATTT | |
| TTACTTTATCCTTCTGTGGCTACAACGTCATTAGTCATTTCTACTGTGACAGTCTCCCTTTG | |
| TTACCTTTGCTTTGTTCAAATACACATGAAATTGAATTGATAATTCTGATCTTTGCAGCTAT | |
| TGATTTGATTTCATCTCTTCTGATAGTTCTTTTATCTTACCTGCTCATCCTTGTAGCCATTCT | |
| CAGGATGAATTCTGCTGGCAGACAAAAGGCTTTTTCTACCTGTGGAGCCCACCTGACAGTG | |
| GTCATAGTGTTCTATGGGACTTTGCTTTTCATGTACGTGCAGCCCAAGTCCAGTCATTCCTT | |
| TGACACTGATAAAGTGGCTTCCATATTTTACACCCTGGTTATCCCCATGTTGAATCCCTTGA | |
| TCTATAGTTTACGAAACAAAGATGTAAAATATGCCCTACGAAGGACATGGAATAACTTATG | |
| TAATATTTTTGTTTAA | |
| AOLFR75 sequences: | |
| MEGKNQTNISEFLLLGFSSWQQQQVLLFALFLCLYLTGLFGNLLILLAIGSDHCLHTPMTTFLA | (SEQ ID NO:139) |
| NLSLVDLCLPSATVIPKMLLNIQTQTQTISYPGCLAQMTTCMMFANMDNFLLTVMAYDRYVAI | |
| CHPLHYSTIMALRLCASLVAAPWVIAILNPLLHTLMMAHLHFCSDNVIHHFFCDINSLLPLSCSD | |
| TSLNQLSVLATVGLIFVVPSVCILVSYILIVSAVMKVPSAQGKLKAFSTCGSHLALVILFYGANT | |
| GVYMSPLSNHSTEKDSAASVIFMVVAPVLNPFIYSLRNNELKGTLKKTLSRPGAVAHACNPSTL | |
| GGRGGWIMRSGDRDHPG | |
| ATGGAAGGGAAAAATCAAACCAATATCTCTGAATTTCTCCTCCTGGGCTTCTCAAGTTGGC | (SEQ ID NO:140) |
| AACAACAGCAGGTGCTACTCTTTGCACTTTTCCTGTGTCTCTATTTAACAGGGCTGTTTGGA | |
| AACTTACTCATCTTGCTGGCCATTGGCTCGGATCACTGCCTTCACACACCCATGTATTTCTT | |
| CCTTGCCAATCTGTCCTTGGTAGACCTCTGCCTTCCCTCAGCCACAGTCCCCAAGATGCTAC | |
| TGAACATCCAAACCCAAACCCAAACCATCTCCTATCCCGGCTGCCTGGCTCAGATGTATTT | |
| CTGTATGATGTTTGCCAATATGGACAATTTTCTTCTCACAGTGATGGCATATGACCGTTAC | |
| GTGGCCATCTGTCACCCTTTACATTACTCCACCATTATGGCCCTGCGCCTCTGTGCCTCTCT | |
| GGTAGCTGCACCTTGGGTCATTGCCATTTTGAACCCTCTCTTGCAGACTCTTATGATGGCCC | |
| ATCTGCACTTCTGCTCTGATAATGTTATCCACCATTTCTTCTGTGATATCAACTCTCTCCTC | |
| CCTCTGTCCTGTTCCGACACCAGTCTTAATCAGTTGAGTGTTCTGGCTACGGTGGGGCTGA | |
| TCTTTGTGGTACCTTCAGTGTGTATCCTGGTATCCTATATCCTCATTGTTTCTGCTGTGATG | |
| AAAGTCCCTTCTGCCCAAGGAAAACTCAAGGCTTTCTCTACCTGTGGATCTCACCTTGCCTT | |
| GGTCATTCTTTTCTATGGAGCAAACACAGGGGTCTATATGAGCCCCTTATCCAATCACTCT | |
| ACTGAAAAAGACTCAGCCGCATCAGTCATTTTTATGGTTGTAGCACCTGTGTTGAATCCAT | |
| TCATTTACAGTTTAAGAAACAATGAACTGAAGGGGACTTTAAAAAAGACCCTAAGCCGGC | |
| CGGGCGCGGTGGCTCACGCCTGTAATCCCAGCACTTTGGGAGGCCGAGGCGGGTGGATCA | |
| TGAGGTCAGGAGATCGAGACCATCCTGGCTAA | |
| AOLFR76 sequences: | |
| MENNTEVSEFILLGLTNAPELQVPLFIMFTLIYLITLTGNLGMIILILLDSHLHTPMTTFLSNLSLA | (SEQ ID NO:141) |
| GIGYSSAVTPKVLTGLLIEDKAISYSACAAQMFFCAVFATVENYLLSSMAYDRYAAVCNPLHY | |
| TTTMTTRVCACLAIGCYVIGFLNASIQIGDTFRLSFCMSNVIHHFFCDKPAVITLTCSEKHISELIL | |
| VLISSFNVFFALLVTLISYLFILITILKRHTGKGYQKPLSTCGSHLIAIFLFYITVIIMYIRPSSSHSM | |
| DTDKIASVFYTMIIPMLSPIVYTLRNKDVKNAFMKVVEKAKYSLDSVF | |
| ATGGAGAATAATACAGAGGTGAGTGAATTCATCCTGCTTGGTCTAACCAATGCCCCAGAA | (SEQ ID NO:142) |
| CTACAGGTTCCCCTCTTTATCATGTTTACCCTCATCTACCTCATCACTCTGACTGGGAACCT | |
| GGGGATGATCATATTAATCCTGCTGGACTCTCATCTCCACACTCCCATGTACTTTTTTCTCA | |
| GTAACCTGTCTCTTGCAGGCATTGGTTACTCCTCAGCTGTCACTCCAAAGGTTTTAACTGG | |
| GTTGCTTATAGAAGACAAAGCCATCTCCTACAGTGCCTGTGCTGCTCAGATGTTCTTTTGT | |
| GCAGTCTTTGCCACTGTGGAAAATTACCTCTTGTCCTCAATGGCCTATGACCGCTACGCAG | |
| CAGTGTGTAACCCCCTACATTATACCACCACCATGACAACACGTGTGTGTGCTTGTCTGGC | |
| TATAGGCTGTTATGTCATTGGTTTTCTGAATGCTTCTATCCAAATTGGAGATACATTTCGCC | |
| TCTCTTTCTGCATGTCCAATGTGATTCATCACTTTTTCTGTGACAAACCAGCAGTCATTACT | |
| CTGACCTGCTCTGAGAAACACATTAGTGAGTTGATTCTTGTTCTTATATCAAGTTTTAATGT | |
| CTTTTTTGCACTTCTTGTTACCTTGATTTCCTATCTGTTCATATTGATCACCATTCTTAAGAG | |
| GCACACAGGTAAGGGATACCAGAAGCCTTTATCTACCTGTGGTTCTCACCTCATTGCCATT | |
| TTCTTATTTTATATAACTGTCATCATCATGTACATACGACCAAGTTCCAGTCATTCCATGGA | |
| CACAGACAAAATTGCATCTGTGTTCTACACTATGATCATCCCCATGCTCAGTCCTATAGTCT | |
| ATACCCTGAGGAACAAAGACGTGAAGAATGCATTCATGAAGGTTGTTGAGAAGGCAAAAT | |
| ATTCTCTAGATTCAGTCTTTTAA | |
| AOLFR77 sequences: | |
| MGDVNQSVASDFILVGLFSHSGSRQLLFSLVAVMFVIGLLGNTVLLFLIRVDSRLHTPMTTLLS | (SEQ ID NO:143) |
| QLSLFDIGCPMVTIPKMASDFLRGEGATSYGGGAAQIFFLTLMGVAEGVLLVLMSYDRYVAVC | |
| QPLQYPVLMRRQVCLLMMGSSWVVGVLNASIQTSITLHFPYCASRIVDHFFCEVPALLKLSCA | |
| DTCAYEMALSTSGVLILMLPLSLIATSYGHVLQAVLSMRSEEARHKAVTTCSSHITVVGLFYGA | |
| AVFMYMVPCAYHSPQQDNVVSLFYSLVTPTLNPLIYSLRNPEVWMALVKVLSRAGLRQMC | |
| ATGGGGGATGTGAATCAGTCGGTGGCCTCAGACTTCATTCTGGTGGGCCTCTTCAGTCACT | (SEQ ID NO:144) |
| +G | |
| CAGGATCACGCCAGCTCCTCTTCTCCCTGGTGGCTGTCATGTTTGTCATAGGCCTTCTGGGC | |
| AACACCGTTCTTCTCTTCTTGATCCGTGTGGACTCCCGGCTCCACACACCCATGTACTTCCT | |
| GCTCAGCCAGCTCTCCCTGTTTGACATTGGCTGTCCCATGGTCACCATCCCCAAGATGGCA | |
| TCAGACTTTCTGCGGGGAGAAGGTGCCACCTCCTATGGAGGTGGTGCAGCTCAAATATTCT | |
| TCCTCACACTGATGGGTGTGGCTGAGGGCGTCCTGTTGGTCCTCATGTCTTATGACCGTTA | |
| TGTTGCTGTGTGCCAGCCCCTGCAGTATCCTGTACTTATGAGACGCCAGGTATGTCTGCTG | |
| ATGATGGGCTCCTCCTGGGTGGTAGGTGTGCTCAACGCCTCCATCCAGACCTCCATCACCC | |
| TGCATTTTCCCTACTGTGCCTCCCGTATTGTGGATCACTTCTTCTGTGAGGTGCCAGCCCTA | |
| CTGAAGCTCTCCTGTGCAGATACCTGTGCCTACGAGATGGCGCTGTCCACCTCAGGGGTGC | |
| TGATCCTAATGCTCCCTCTTTCCCTCATCGCCACCTCCTACGGCCACGTGTTGCAGGCTGTT | |
| CTAAGCATGCGCTCAGAGGAGGCCAGACACAAGGCTGTCACCACCTGCTCCTCGCACATCA | |
| CGGTAGTGGGGCTCTTTTATGGTGCCGCCGTGTTCATGTACATGGTGCCTTGCGCCTACCA | |
| CAGTCCACAGCAGGATAACGTGGTTTCCCTCTTCTATAGCCTTGTCACCCCTACACTCAAC | |
| CCCCTTATCTACAGTCTGAGGAATCCGGAGGTGTGGATGGCTTTGGTCAAAGTGCTTAGCA | |
| GAGCTGGACTCAGGCAAATGTGCTGA | |
| AOLFR78 sequences: | |
| MSPDGNHSSDPTEFVLAGLPNLNSARVELFSVFLLVYLLNLTGNVLIVGVVRADTRLQTPMTT | (SEQ ID NO:145) |
| FLGNLSCLEILLTSVIIPKMLSNFLSRQHTISFAACITQFTTTTFLGASEFLLLAVMSADRYLAICH | |
| PLRYPLLMSGAVCFRVALACWVGGLVPVLGPTVAVALLPFCKQGAVVQHFFCDSGPLLRLAC | |
| TNTKKLEETDFVLASLVIVSSLLITAVSYGLIVLAVLSIPSASGRQKAFSTCTSHLIVVTLFYGSAI | |
| FLYVRPSQSGSVDTNWAVTVITTFVTPLLNPFIYALRNEQVKEALKDMFRKVVAGVLGNLLLD | |
| KCLSEKAVK | |
| ATGAGTCCTGATGGGAACCACAGTAGTGATCCAACAGAGTTCGTCCTGGCAGGGCTCCCA | (SEQ ID NO:146) |
| AATCTCAACAGCGCAAGAGTGGAATTATTTTCTGTGTTTCTTCTTGTCTATCTCCTGAATCT | |
| GACAGGCAATGTGTTGATTGTGGGGGTGGTAAGGGCTGATACTCGACTACAGACCCCTAT | |
| GTACTTCTTTCTGGGTAACCTGTCCTGCCTAGAGATACTGCTCACTTCTGTCATCATTCCAA | |
| AGATGCTGAGCAATTTCCTCTCAAGGCAACACACTATTTCCTTTGCTGCATGTATCACCCA | |
| ATTCTATTTCTACTTCTTTCTCGGGGCCTCCGAGTTCTTACTGTTGGCTGTCATGTCTGCGG | |
| ATCGCTACCTGGCCATCTGTCATCCTCTGCGCTACCCCTTGCTCATGAGTGGGGCTGTGTG | |
| CTTTCGTGTGGCCTTGGCCTGCTGGGTGGGGGGACTCGTCCCTGTGCTTGGTCCCACAGTG | |
| GCTGTGGCCTTGCTTCCTTTCTGTAAGCAGGGTGCTGTGGTACAGCACTTCTTCTGCGACA | |
| GTGGCCCACTGCTCCGCCTGGCTTGCACCAACACCAAGAAGCTGGAGGAGACTGACTTTGT | |
| CCTGGCCTCCCTCGTCATTGTATCTTCCTTGCTGATCACTGCTGTGTCCTACGGCCTCATTG | |
| TGCTGGCAGTCCTGAGCATCCCCTCTGCTTCAGGCCGTCAGAAGGCCTTCTCTACCTGTAC | |
| CTCCCACTTGATAGTGGTGACCCTCTTCTATGGAAGTGCCATTTTTCTCTATGTGCGGCCAT | |
| CGCAGAGTGGTTCTGTGGACACTAACTGGGCAGTGACAGTAATAACGACATTTGTGACAC | |
| CACTGTTGAATCCATTCATCTATGCCTTACGTAATGAGCAAGTCAAGGAAGCTTTGAAGGA | |
| CATGTTTAGGAAGGTAGTGGCAGGCGTTTTAGGGAATCTTTTACTTGATAAATGTCTCAGT | |
| GAGAAAGCAGTAAAGTAA | |
| AOLFR79 sequences: | |
| MTPGELALASGNHTPVTKFILQGFSNYPDLQELLFGAILLIYAITVVGNLGMMALIFTDSHLQSP | (SEQ ID NO:147) |
| MTTFLNVLSFLDICYSSVVTPKLLVNFLVSDKSISFEGCVVQLAFFVVHVTAESFLLASMAYDR | |
| FLAICQPLHYGSIMTRGTCLQLVAVSYAFGGANSAIQTGNVFALPFCGPNQLTHYYCDIPPLLH | |
| LACANTATARVVLYVFSALVTLLPAAVILTSYCLVLVAIGRMRSVAGREKDLSTCASHFLAIAI | |
| FYGTVVFTYVQPHGSTNNTNGQVVSVFYTIIIPMLNPFIYSLRNKEVKGALQRKLQVNIFPG | |
| ATGACACCTGGAGAACTAGCCCTTGCCAGTGGCAACCACACCCCAGTCACCAAGTTCATCT | (SEQ ID NO:148) |
| TGCAGGGATTCTCCAATTATCCAGACCTCCAGGAGCTTCTCTTCGGAGCCATCCTGCTCAT | |
| CTATGCCATAACAGTGGTGGGCAACTTGGGAATGATGGCACTCATCTTCACAGACTCCCAT | |
| CTCCAAAGCCCAATGTATTTCTTCCTCAATGTCCTCTCGTTTCTTGATATTTGTTACTCTTCT | |
| GTGGTCACACCTAAGCTCTTGGTCAACTTCCTGGTCTCTGACAAGTCCATCTCTTTTGAGG | |
| GCTGTGTGGTCCAGCTCGCCTTCTTTGTAGTGCATGTGACAGCTGAGAGCTTCCTGCTGGC | |
| CTCCATGGCCTATGACCGCTTCCTAGCCATCTGTCAACCCCTCCATTATGGTTCTATCATGA | |
| CCAGGGGGACCTGTCTCCAGCTGGTAGCTGTGTCCTATGCATTTGGTGGAGCCAACTCCGC | |
| TATCCAGACTGGAAATGTCTTTGCCCTGCCTTTCTGTGGGCCCAACCAGCTAACACACTAC | |
| TACTGTGACATACCACCCCTTCTCCACCTGGCTTGTGCCAACACAGCCACAGCAAGAGTGG | |
| TCCTCTATGTCTTTTCTGCTCTGGTCACCCTTCTGCCTGCTGCAGTCATTCTCACCTCCTACT | |
| GCTTGGTCTTGGTGGCCATTGGGAGGATGCGCTCAGTAGCAGGGAGGGAGAAGGAGCTCT | |
| CCACTTGTGCCTCCCACTTTCTGGCCATTGCCATTTTCTATGGCACTGTGGTTTTCACCTAT | |
| GTTCAGCCCCATGGATCTACTAACAATACCAATGGCCAAGTAGTGTCCGTCTTCTACACCA | |
| TCATAATTCCCATGCTCAATCCCTTCATCTATAGCCTCCGCAACAAGGAGGTGAAGGGCGC | |
| TCTGCAGAGGAAGCTTCAGGTCAACATCTTTCCCGGCTGA | |
| AOLFR80 sequences: | |
| MEGINKTAKMQFFFRPFSPDPEVQMLIFVVFLMMYLTSLGGNATIAVIVQINHSLHTPMTTFLA | (SEQ ID NO:149) |
| NLAVLEIFYTSSITPLALANLLSMGKTPVSITGCGTQMFFFVFLGGADCVLLVVMAYDRFIAICH | |
| PLRYRLIMSWSLCVELLVGSLVLGFLLSLPLTILIFHLPFCHNDEIYHFYCDMPAVMRLACADTR | |
| VHKTALYIISFIVLSIPLSLISISYVFIVVAILRIRSAEGRQQAYSTCSSHILVVLLQYGCTSFIYLSPS | |
| SSYSPEMGRVVSVAYTFITPILNPLIYSLRNKELKDALRKALRKF | |
| ATGGAAGGAATAAATAAAACTGCAAAGATGCAGTTTTTCTTTCGTCCATTCTCACCTGACC | (SEQ ID NO:150) |
| CTGAGGTCCAGATGCTGATTTTTGTGGTCTTCCTGATGATGTATCTGACCAGCCTCGGTGG | |
| AAATGCTACAATTGCAGTCATTGTTCAGATCAATCATTCCCTCCACACCCCCATGTACTTTT | |
| TCCTGGCTAATCTGGCAGTTCTAGAAATCTTCTATACATCTTCCATCACCCCATTGGCCTTG | |
| GCAAACCTCCTTTCAATGGGCAAAACTCCTGTTTCCATCACGGGATGTGGCACCCAGATGT | |
| TTTTCTTTGTCTTCTTGGGTGGGGCTGATTGTGTCCTGCTGGTAGTCATGGCTTATGACCGG | |
| TTTATAGCGATCTGTCACCCTCTGCGATACAGGCTCATCATGAGCTGGTCCTTGTGTGTGG | |
| AGCTGCTGGTAGGCTCCTTGGTGCTGGGGTTCCTGTTGTCACTGCCACTCACCATTTTAATC | |
| TTCCATCTCCCATTCTGCCACAATGATGAGATCTACCACTTCTACTGTGACATGCCTGCAGT | |
| CATGCGCCTGGCTTGTGCAGACACACGCGTTCACAAGACTGCTCTGTATATCATCAGCTTC | |
| ATCGTCCTTAGCATCCCCCTCTCATTGATCTCCATCTCCTATGTCTTCATCGTGGTAGCCAT | |
| TTTACGGATCCGGTCAGCAGAAGGGCGCCAGCAAGCCTACTCTACCTGCTCTTCTCACATC | |
| TTAGTGGTCCTCCTGCAGTATGGCTGCACCAGCTTTATATACTTGTCCCCCAGTTCCAGCTA | |
| CTCTCCTGAGATGGGCCGGGTGGTATCTGTGGCCTACACATTTATCACTCCCATTTTAAAC | |
| CCCTTGATCTATAGTTTGAGGAACAAGGAACTGAAAGATGCCCTAAGGAAAGCATTGAGA | |
| AAATTCTAG | |
| AOLFR81 sequences: | |
| MGVKNHSTVTEFLLSGLTEQAELQLPLFCLFLGIYTVTVVGNLSMISIIRLNRQLHTPMYTTLSS | (SEQ ID NO:151) |
| LSFLDFCYSSVITPKMMKLWMESHLIVPETRPSPRMMSNQTLVTEFILQGFSEHPEYRVFLFSCF | |
| LFLYSGALTGNVLITLAITFNPGLHAPMTTFLLNLATMDIICTSSIMPKALASLVSEESSISYGGC | |
| MAQLTTLTWAASSELLLLTVMAYDRYAAICHPLHYSSMMSKVFCSGLATAVWLLCAVNTAIH | |
| TGLMLRLDFCGPNVIIHFFCEVIPPLLLLSCSSTYVNGVMIVLADAFYGIVNFLMTIASYGFIVSSI | |
| LKVKTAWGRQKAFSTCSSHLTVVCMYYTAVFYAYISPVSGYSAGKSKLAGLLYTVLSPTLNPL | |
| IYTLRNKEVKAALRKLFPFFRN | |
| ATGAAGCTGTGGATGGAGAGTCACCTGATAGTCCCAGAAACCCGTCCCAGCCCAAGGATG | (SEQ ID NO:152) |
| ATGAGTAACCAGACGTTGGTAACCGAGTTCATCCTGCAGGGCTTTTCGGAGCACCCAGAAT | |
| ACCGGGTGTTCTTATTCAGCTGTTTCCTCTTCCTCTACTCTGGGGCCCTCACAGGTAATGTC | |
| CTCATCACCTTGGCCATCACGTTCAACCCTGGGCTCCACGCTCCTATGTACTTTTTCTTACT | |
| CAACTTGGCTACTATGGACATTATCTGCACCTCTTCCATCATGCCCAAGGCGCTGGCCAGT | |
| CTGGTGTCGGAAGAGAGCTCCATCTCCTACGGGGGCTGCATGGCCCAGCTCTATTTCCTCA | |
| CGTGGGCTGCATCCTCAGAGCTGCTGCTCCTCACGGTCATGGCCTATGACCGGTACGCAGC | |
| CATCTGCCACCCGCTGCATTACAGCAGCATGATGAGCAAGGTGTTCTGCAGCGGGCTGGCC | |
| ACAGCCGTGTGGCTGCTCTGCGCCGTCAACACGGCCATCCACACGGGGCTGATGCTGCGCT | |
| TGGATTTCTGTGGCCCCAATGTCATTATCCATTTCTTCTGCGAGGTCCCTCCCCTGCTGCTT | |
| CTCTCCTGCAGCTCCACCTACGTCAACGGTGTCATGATTGTCCTGGCGGATGCTTTCTACG | |
| GCATAGTGAACTTCCTGATGACCATCGCGTCCTATGGCTTCATCGTCTCCAGCATCCTGAA | |
| GGTGAAGACTGCCTGGGGGAGGCAGAAAGCCTTCTCCACCTGCTCTTCCCACCTCACCGTG | |
| GTGTGCATGTATTACACCGCTGTCTTCTACGCCTACATAAGCCCGGTCTCTGGCTACAGCG | |
| CAGGGAAGAGCAAGTTGGCTGGCCTGCTGTACACTGTGCTGAGTCCTACCCTCAACCCCCT | |
| CATCTATACTTTGAGAAACAAGGAGGTCAAAGCAGCCCTCAGGAAGCTTTTCCCTTTCTTC | |
| AGAAATTAA | |
| AOLFR82 sequences: | |
| MQLNNNVTEFILLGLTQDPFWKKIVFVIFLRLYLGTLLGNLLIIISVKASQALKNPMFFFLFYLSL | (SEQ ID NO:153) |
| SDTCLSTSIAPRMIVDALLKKTTISFSECMIQVFSSHVFGCLEIFILILTAVDRYVDICKPLHYMTII | |
| SQWVCGVLMAVAWVGSCVHSLVQIFLALSLPFCGPNVINHCFCDLQPLLKQACSETYVVNLLL | |
| VSNSGAICAVSYVMLIFSYVILFLHSLRNHSAEVIKKALSTCVSHIIVVILFFGPCIFMYTCPATVFP | |
| MDKMIAVFYTVGTSFLNPVIYTLKNTEVKSAMRKLWSKKLITDDKR | |
| ATGCAACTGAATAATAATGTGACTGAGTTCATTCTGCTTGGATTGACACAGGATCCTTTTT | (SEQ ID NO:154) |
| GGAAGAAAATAGTGTTTGTTATTTTTTTGCGTCTCTACTTGGGAACACTGTTGGGTAATTT | |
| GCTAATCATTATTAGTGTCAAGGCCAGCCAGGCACTTAAGAACCCAATGTTCTTCTTCCTT | |
| TTCTACTTATCTTTATCTGATACTTGCCTCTCTACTTCCATAGCCCCTAGAATGATTGTGGA | |
| TGCCCTTTTGAAGAAGACAACTATCTCCTTCAGCGAGTGCATGATCCAAGTCTTTTCATCC | |
| CATGTCTTTGGCTGCCTGGAGATCTTCATCCTCATCCTCACGGCTGTTGACCGCTATGTGGA | |
| CATCTGTAAGCCCCTGCACTACATGACCATCATAAGCCAGTGGGTCTGTGGTGTTTTGATG | |
| GCTGTGGCCTGGGTGGGATCCTGTGTGCATTCTTTAGTTCAGATTTTTCTTGCCCTGAGTTT | |
| GCCATTCTGTGGCCCCAATGTGATCAATCACTGTTTCTGTGACTTGCAGCCCTTGTTGAAA | |
| CAAGCCTGTTCAGAAACCTATGTGGTTAACCTACTCCTGGTTTCCAATAGTGGGGCCATTT | |
| GTGCAGTGAGTTATGTCATGCTAATATTCTCCTATGTCATCTTCTTGCATTCTCTGAGAAAC | |
| CACAGTGCTGAAGTGATAAAGAAAGCACTTTCCACATGTGTCTCCCACATCATTGTGGTCA | |
| TCTTGTTCTTTGGACCTTGCATATTTATGTACACATGCCCTGCAACCGTATTCCCCATGGAT | |
| AAGATGATAGCTGTATTTTATACAGTTGGAACATCTTTTCTCAACCCTGTGATTTACACGCT | |
| GAAGAATACAGAAGTGAAAAGTGCCATGAGGAAGCTTTGGAGCAAGAAATTGATCACAGA | |
| TGACAAAAGATAA | |
| AOLFR83 sequences: | |
| MGNWTAAVTEFVLLGFSLSREVELLLLVLLLPTFLLTLLGNLLIISTVLSCSRLHTPMTTFLCNL | (SEQ ID NO:155) |
| SILDILFTSVISPKVLANLGSRDKTISFAGCITQCTTTTFLGTVEFLLLTVMSYDRYATICCPLRYT | |
| TIMRPSVCIGTVVFSWVGGFLSVLFPTILISQLPFCGSNIINHFFCDSGPLLALACADTTAIELMDF | |
| MLSSMVILCCIVLVAYSYTYIILTIVRIPSASGRKKAFNTCASHLTIVIIPSGITVFIYVTPSQKEYL | |
| EINKIPLVLSSVVTPFLNPFIYTLRNDTVQGVLRDVWVRVRGVFEKRMRAVLRSRLSSNKDHQ | |
| GRACSSPPCVYSVKLQC | |
| ATGGGTAACTGGACTGCAGCGGTGACTGAGTTTGTTCTGCTGGGGTTTTCCCTGAGCAGGG | (SEQ ID NO:156) |
| AGGTGGAGCTGCTGCTCCTGGTGCTCCTGCTGCCCACGTTCCTGCTGACTCTTCTGGGGAA | |
| CCTGCTCATCATCTCCACTGTGCTGTCCTGCTCCCGCCTCCACACCCCCATGTACTTCTTCT | |
| TGTGCAACCTCTCTCTCCTGGACATCCTCTTCACCTCAGTCATCTCTCCAAAAGTGTTGGCC | |
| AACTTAGGATCTAGGGATAAAACCATCTCCTTTGCCGGATGTATCACCCAGTGCTATTTCT | |
| ACTTTTTCTTGGGCACAGTTGAGTTCCTCCTGCTGACGGTCATGTCCTATGACCGTTATGCC | |
| ACCATCTGCTGCCCCCTGCGGTACACCACCATCATGAGACCTTCTGTCTGCATTGGGACCG | |
| TTGTATTCTCTTGGGTGGGAGGCTTCCTGTCTGTGCTCTTTCCAACCATCCTCATCTCCCAG | |
| CTGCCCTTCTGTGGCTCCAATATCATTAACCACTTCTTCTGTGACAGTGGACCCTTGCTGGC | |
| CCTGGCCTGTGCAGACACCACTGCCATCGAGCTGATGGATTTTATGCTTTCTTCCATGGTG | |
| ATCCTCTGCTGCATAGTCCTCGTGGCCTATTCCTATACGTACATCATCTTGACCATAGTGCG | |
| CATTCCTTCTGCAAGTGGAAGGAAGAAGGCCTTTAATACCTGTGCTTCCCACCTGACCATA | |
| GTCATCATTCCTAGTGGCATCACTGTGTTTATCTATGTGACTCCCTCCCAGAAAGAATATCT | |
| GGAGATCAACAAGATCCCTTTGGTTCTGAGCAGTGTGGTGACTCCATTCCTCAACCCCTTT | |
| ATATATACTCTGAGGAATGACACAGTGCAGGGAGTCCTCAGGGATGTGTGGGTCAGGGTT | |
| CGAGGAGTTTTTGAAAAGAGGATGAGGGCAGTGCTGAGAAGCAGATTATCCTCCAACAAA | |
| GACCACCAAGGAAGGGCTTGCTCTTCTCCACCATGTGTCTATTCTGTAAAGCTCCAGTGTT | |
| AG | |
| AOLFR85 sequences: | |
| MGAKNNVTEFVLFGLFESREMQHTCFVVFFLFHVLTVLGNLLVIITINARKTLKSPMTTFLSQL | (SEQ ID NO:157) |
| SFADICYPSTTIPKMIADTFVEHKIISFNGCMTQLFSAHFFGGTEIFLLTAMAYDRYVAICRPLHY | |
| TAIMDCRKCGLLAGASWLAGFLHSILQTLLTVQLPFCGPNEIDNFFCDVHPLLKLACADTYMV | |
| GLIVVANSGMISLASFFILIISYVIILLNLRSQSSEDRRKAVSTCGSHVITVLLVLMPPMFMYIRPS | |
| TTLAADKLIILFNIVMPPLLNPLIYTLRNNDVKNAMRKLFRVKRSLGEK | |
| ATGGGTGCCAAGAACAATGTGACTGAGTTTGTTTTATTTGGCCTTTTTGAGAGCAGAGAGA | (SEQ ID NO:158) |
| TGCAGCATACATGCTTTGTGGTATTCTTCCTCTTTCATGTGCTCACTGTCCTGGGGAACCTT | |
| CTGGTCATCATCACCATCAATGCTAGAAAGACCCTGAAGTCTCCCATGTATTTCTTCCTGA | |
| GCCAGTTGTCTTTTGCTGACATATGTTATCCATCCACTACCATACCCAAGATGATTGCTGAC | |
| ACTTTTGTGGAGCATAAGATCATCTCCTTCAATGGCTGCATGACCCAGCTCTTTTCTGCCCA | |
| CTTCTTTGGTGGCACTGAGATCTTCCTCCTTACAGCCATGGCCTATGACCGCTATGTGGCC | |
| ATCTGTAGGCCCCTGCACTACACAGCCATCATGGATTGCCGGAAGTGTGGCCTGCTAGCGG | |
| GGGCCTCCTGGTTAGCTGGCTTCCTGCATTCCATCCTGCAGACCCTCCTCACGGTTCAGCTG | |
| CCTTTTTGTGGGCCCAATGAGATAGACAACTTCTTCTGTGATGTTCATCCCCTGCTCAAGTT | |
| GGCCTGTGCAGACACCTACATGGTAGGTCTCATCGTGGTGGCCAACAGCGGTATGATTTCT | |
| TTAGCATCCTTTTTTATCCTTATCATTTCCTATGTTATCATCTTACTGAACCTAAGAAGCCA | |
| GTCATCTGAGGACCGGCGTAAGGCTGTCTCCACATGTGGCTCACACGTAATCACTGTCCTT | |
| TTGGTTCTCATGCCCCCCATGTTCATGTACATTCGTCCCTCCACCACCCTGGCTGCTGACAA | |
| ACTTATCATCCTCTTTAACATTGTGATGCCACCTTTGCTGAACCCTTTGATCTATACACTAA | |
| GGAACAACGATGTGAAAAATGCCATGAGGAAGCTGTTTAGGGTCAAGAGGAGCTTAGGGG | |
| AGAAGTGA | |
| AOLFR86 sequences: | |
| MQLVLLLMFLLVFIGNTAPAFSVTLESMDIPQNITEFFMLGLSQNSEVQRVLFVVFLLIIYVVTVC | (SEQ ID NO:159) |
| GNMLIVVTITSSPTLASPVTTFLANLSFIDTFYSSSMAPKLIADSLYEGRTISYECCMAQLFGAHF | |
| LGGVEIILLTVMAYDRYVAICKPLHNTTIMTRHLCAMLVGVAWLGGFLHSLVQLLLVLWLPFC | |
| GPNVINHFACDLYPLLEVACTNTYVIGLLVVANSGLICLLNFLMLAASYIVILYSLRSHSADGRC | |
| KALSTCGAHFIVVALFFVPCIFTYVHPFSTLPIDKNMALFYGILTPMLNPLIYTLRNEEVKNAMR | |
| KLFTW | |
| ATGCAATTAGTTCTATTACTTATGTTTCTCCTTGTCTTTATAGGCAATACTGCACCTGCATT | (SEQ ID NO:160) |
| CTCAGTGACCTTGGAATCTATGGACATACCACAAAATATCACAGAATTTTTCATGCTGGGG | |
| CTCTCACAGAACTCAGAGGTACAGAGAGTTCTCTTTGTGGTCTTTTTGCTGATCTATGTGG | |
| TCACGGTTTGTGGCAACATGCTCATTGTGGTCACTATCACCTCCAGCCCCACGCTGGCTTC | |
| CCCTGTGTATTTTTTCCTGGCCAACCTATCCTTTATTGACACCTTTTATTCTTCTTCTATGGC | |
| TCCTAAACTCATTGCTGACTCATTGTATGAGGGGAGAACCATCTCTTATGAGTGCTGCATG | |
| GCTCAGCTCTTTGGAGCTCATTTTTTGGGAGGTGTTGAGATCATTCTGCTCACAGTGATGG | |
| CTTATGACCGCTATGTGGCCATCTGTAAGCCCCTGCACAATACTACCATCATGACCAGGCA | |
| TCTCTGTGCCATGCTTGTAGGGGTGGCTTGGCTTGGGGGCTTCCTGCATTCATTGGTTCAG | |
| CTCCTCCTGGTCCTTTGGTTGCCCTTCTGTGGGCCCAATGTGATCAATCACTTTGCCTGTGA | |
| CTTGTACCCTTTGCTGGAAGTTGCCTGCACCAATACGTATGTCATTGGTCTGCTGGTGGTT | |
| GCCAACAGTGGTTTAATCTGCCTGTTGAACTTCCTCATGCTGGCTGCCTCCTACATTGTCAT | |
| CCTGTACTCCTTGAGGTCCCACAGTGCAGATGGGAGATGCAAAGCCCTCTCCACCTGTGGA | |
| GCCCACTTCATTGTTGTTGCCTTGTTCTTTGTGCCCTGTATATTTACTTATGTGCATCCATTT | |
| TCTACTTTACCTATAGACAAAAATATGGCATTATTTTATGGTATTCTGACACCTATGTTGAA | |
| TCCACTCATTTATACCCTGAGAAATGAAGAGGTAAAAAATGCCATGAGAAAGCTCTTTACA | |
| TGGTAA | |
| AOLFR87 sequences: | |
| MNNIAQLSLGFIDLGIPSVLQKIILTKIILLFKMYVSNCNPCAIHRKINYPNTKLDFEQVNNITEFI | (SEQ ID NO:161) |
| LLGLTQNAEAQKLLFAVFTLITTLTMVDNLIIVVTITTSPALDSPVTTFLSFFSFIDGCSSSTMAP | |
| KMIFDLLTEKKTISFSGCMTQLFVEHFFGGVEIILLVVMAYDCYVAICKPLYYLITMNRQVCGL | |
| LVAMAWVGGFLHALIQMLLIVWLPFCGPNVIDHFICDLFPLLKLSCTDTHVFGLFVAANSGLM | |
| CMLIFSILITSYVLILCSQRKALSTCAFHITVVVLFFVPCILVYLRPMITFPIDKAVSVFYTVVTPM | |
| LNPLIYTLRNTEVKNAMKQLWSQIIWGNNLCD | |
| ATGAATAACATAGCTCAACTTAGTCTTGGGTTTATAGATTTAGGGATTCCATCAGTGTTAC | (SEQ ID NO:162) |
| AGAAAATAATCCTGACCAAAATTATTTTATTGTTCAAAATGTATGTGTCAAATTGCAATCC | |
| TTGTGCTATTCACAGAAAAATCAATTATCCAAATACCAAACTGGATTTCGAGCAAGTGAAC | |
| AACATAACGGAATTCATCTTGCTTGGCCTGACACAGAACGCAGAGGCACAGAAACTCTTGT | |
| TTGCTGTGTTTACACTCATCTACTTTCTCACCATGGTAGACAACCTAATCATTGTGGTGACA | |
| ATCACCACCAGCCCAGCCCTGGACTCCCCCGTGTATTTTTTTCTGTCTTTCTTTTCCTTCAT | |
| AGATGGCTGCTCCTCTTCTACCATGGCCCCCAAAATGATATTTGACTTACTCACTGAAAAG | |
| AAAACTATTTCCTTCAGTGGGTGCATGACCCAGCTCTTTGTAGAACATTTCTTTGGGGGAG | |
| TTGAGATCATTCTGCTCGTGGTGATGGCCTATGACTGCTATGTGGCCATCTGCAAGCCCCT | |
| GTACTACCTGATCACAATGAACAGGCAGGTATGTGGCCTCCTGGTGGCCATGGCATGGGTC | |
| GGGGGATTTCTTCACGCTCTGATTCAAATGCTTTTAATAGTCTGGCTGCCCTTCTGTGGCCC | |
| CAATGTCATTGACCATTTCATCTGTGACCTTTTCCCTCTGCTAAAACTCTCCTGCACTGACA | |
| CTCACGTCTTTGGACTCTTTGTTGCCGCCCAACAGTGGGCTGATGTGTATGCTCATTTTTTCT | |
| ATTCTTATTACCTCTTACGTCCTAATCCTCTGCTCACAGCGGAAGGCTCTCTCTACCTGCGC | |
| CTTCCATATCACTGTAGTCGTCCTATTCTTTGTTCCCTGTATATTGGTGTACCTTCGACCCA | |
| TGATCACCTTCCCTATTGATAAAGCTGTGTCTGTGTTTTATACTGTGGTAACACCCATGTTA | |
| AACCCTTTAATCTACACCCTCAGAAACACAGAGGTGAAAAATGCCATGAAGCAGCTCTGG | |
| AGCCAAATAATCTGGGGTAACAATTTGTGTGATTAG | |
| AOLFR88 sequences: | |
| MWQKNQTSLADFILEGLFDDSLTHLFLFSLTMVVFLIAVSGNTLTILLICIDPQLHTPMTTLLSQ | (SEQ ID NO:163) |
| LSLMDLMHVSTIILKMATNYLSGKKSISFVGCATQHFLYLCLGGAECFLLAVMSYDRYVAICH | |
| PLRYAVLMNKKVGLMMAVMSWLGASVNSLIHMAILMHFPFCGPRKVYCEFPAVVKLVC | |
| GDITVYETTVYISSILLLLPIFLISTSYVFILQSVIQMRSSGSKRNAFATCGSHLTVVSLWFGACIFS | |
| YMRPRSQCTLLQNKVGSVFYSIITPTLNSLIYTLRNKDVAKALRRVLRRDVITQCIQRLQLWLP | |
| RV | |
| ATGTGGCAGAAGAATCAGACCTCTCTGGCAGACTTCATCCTTGAGGGGCTCTTCGATGACT | (SEQ ID NO:164) |
| CCCTTACCCACCTTTTCCTTTTCTCCTTGACCATGGTGGTCTTCCTTATTGCGGTGAGTGGC | |
| AACACCCTCACCATTCTCCTCATCTGCATTGATCCCCAGCTTCATACACCAATGTATTTCCT | |
| GCTCAGCCAGCTCTCCCTCATGGATCTGATGCATGTCTCCACAATCATCCTGAAGATGGCT | |
| ACCAACTACCTATCTGGCAAGAAATCTATCTCCTTTGTGGGCTGTGCAACCCAGCACTTCC | |
| TCTATTTGTGTCTAGGTGGTGCTGAATGTTTTCTCTTAGCTGTCATGTCCTATGACCGCTAT | |
| GTTGCCATCTGTCATCCACTGCGCTATGCTGTGCTCATGAACAAGAAGGTGGGACTGATGA | |
| TGGCTGTCATGTCATGGTTGGGGGCATCCGTGAACTCCCTAATTCACATGGCGATCTTGAT | |
| GCACTTCCCTTTCTGTGGGCCTCGGAAAGTCTACCACTTCTACTGTGAGTTCCCAGCTGTTG | |
| TGAAGTTGGTATGTGGCGACATCACTGTGTATGAGACCACAGTGTACATCAGCAGCATTCT | |
| CCTCCTCCTCCCCATCTTCCTGATTTCTACATCCTATGTCTTCATCCTTCAAAGTGTCATTCA | |
| GATGCGCTCATCTGGGAGCAAGAGAAATGCCTTTGCCACTTGTGGCTCCCACCTCACGGTG | |
| GTTTCTCTTTGGTTTGGTGCCTGCATCTTCTCCTACATGAGACCCAGGTCCCAGTGCACTCT | |
| ATTGCAGAACAAAGTTGGTTCTGTGTTCTACAGCATCATTACGCCCACATTGAATTCTCTG | |
| ATTTATACTCTCCGGAATAAAGATGTAGCTAAGGCTCTGAGAAGAGTGCTGAGGAGAGAT | |
| GTTATCACCCAGTGCATTCAACGACTGCAATTGTGGTTGCCCCGAGTGTAG | |
| AOLFR89 sequences: | |
| MLDPSISSHTLYLHSLFPQGLRKGTMWQKNQTSLADFILEGLFDDSLTHLFLFSLTMVVLIAVS | (SEQ ID NO:165) |
| GNTLTILLICIDPQLHTPMTTLLSQLSLMDLMHVSTTILKMATNYLSGKKSISFVGCATQHFLYL | |
| CLGGAECFLLAVMSYDRYVAICHPLRYAVLMNKKVGLMMAVMSWLGASVNSLIHMAILMHF | |
| PFCGPRKVYHFYCEFPAVVKLVCGDITVYETTVYISSILLLLPIFLISTSYVFILQSVIQMRSSGSK | |
| RNAFATCGSHLTVVSLWFGACIFSYMRPRSQCTLLQNKVGSVFYSIITPTLNSIYTLRNKDVA | |
| KALRRVLRRDVITQCIQRLQLWLPRV | |
| ATGCTGGACCCCAGTATTTCCAGTCACACTCTTTATCTCCACTCTCTGTTTCCTCAGGGATT | (SEQ ID NO:166) |
| GAGAAAGGGGACAATGTGGCAGAAGAATCAGACCTCTCTGGCAGACTTCATCCTTGAGGG | |
| GCTCTTGGATGACTCCCTTACCCACCTTTTCCTTTTCTCCTTGACCATGGTGGTCTTCCTTAT | |
| TGCGGTGAGTGGCAACACCCTCACCATTCTCCTCATCTGCATTGATCCCCAGCTTCATACA | |
| CCAATGTATTTCCTGCTCAGCCAGCTCTCCCTCATGGATCTGATGCATGTCTCCACAACCAT | |
| CCTGAAGATGGCTACCAACTACCTATCTGGCAAGAAATCTATCTCCTTTGTGGGCTGTGCA | |
| ACCCAGCACTTCCTCTATTTGTGTCTAGGTGGTGCTGAATGTTTTCTCTTAGCTGTCATGTC | |
| CTATGACCGCTATGTTGCCATCTGTCATCCACTGCGCTATGCTGTGCTCATGAACAAGAAG | |
| GTGGGACTGATGATGGCTGTCATGTCATGGTTGGGGGCATCCGTGAACTCCCTAATTCACA | |
| TGGCGATCTTGATGCACTTCCCTTTCTGTGGGCCTCGGAAAGTCTACCACTTCTACTGTGA | |
| GTTCCCAGCTGTTGTGAAGTTGGTATGTGGCGACATCACTGTGTATGAGACCACAGTGTAC | |
| ATCAGCAGCATTCTCCTCCTCCTCCCCATCTTCCTGATTTCTACATCCTATGTCTTCATCCTT | |
| CAAAGTGTCATTCAGATGCGCTCATCTGGGAGCAAGAGAAATGCCTTTGCCACTTGTGGCT | |
| CCCACCTCACGGTGGTTTCTCTTTGGTTTGGTGCCTGCATCTTCTCCTACATGAGACCCAGG | |
| TCCCAGTGCACTCTATTGCAGAACAAAGTTGGTTCTGTGTTCTACAGCATCATTACGCCCA | |
| CATTGAATTCTCTGATTTATACTCTCCGGAATAAAGATGTAGCTAAGGCTCTGAGAAGAGT | |
| GCTGAGGAGAGATGTTATCACCCAGTGCATTCAACGACTGCAATTGTGGTTGCCCCGAGTG | |
| TAG | |
| AOLFR90 sequences: | |
| MFSMTTEALNNFALGCTNLLMTMIPQIDLKQIFLCPNCRLYMIPVGAFIFSLGNMQNQSFVTEF | (SEQ ID NO:167) |
| VLLGLSQNPNVQEIVFVVFLFVYIATVGGNMLIVVTILSSPALLVSPMTTFLGFLSFLDACFSSVI | |
| TPKMIVDSLYVTKTISFEGCMMQLFAEHFFAGVEVIVLTAMAYDRYVAICKPLHYSSIMNRRL | |
| CGILMGVAWTGGLLHSMIQILFTFQLPFCGPNVINHFMCDLYPLLELACTDTHIFGLMVVINSG | |
| FICIINFSLLLVSYAVILLSLRTHSSEGRWKALSTCGSHIAVVILFFVPCIFVYTRPPSAFSLDKMA | |
| AIFYIILNPLLNPLIYTFRNKEVKQAMRRIWNRLMVVSDEKENIKL | |
| ATGTTCTCAATGACAACAGAAGCACTCAATAATTTTGCACTTGGATGTACCAACTTGTTAA | (SEQ ID NO:168) |
| TGACTATGATACCACAAATTGATCTGAAGCAAATTTTCCTTTGTCCTAATTGCAGACTATA | |
| CATGATCCCTGTTGGAGCTTTCATCTTTTCCTTGGGAAACATGCAAAACCAAAGCTTTGTA | |
| ACTGAGTTTGTCCTCCTGGGACTTTCACAGAATCCAAATGTTCAGGAAATAGTATTTGTTG | |
| TATTTTTGTTTGTCTACATTGCAACTGTTGGGGGCAACATGCTAATTGTAGTAACCATTCTC | |
| AGCAGCCCTGCTCTTCTGGTGTCTCCTATGTACTTCTTCTTGGGCTTCCTGTCCTTCCTGGA | |
| TGCGTGCTTCTCATCTGTCATCACCCCAAAGATGATTGTAGACTCCCTCTATGTGACAAAA | |
| ACCATCTCTTTTGAAGGCTGCATGATGCAGCTCTTTGCTGAACACTTCTTTGCTGGGGTGG | |
| AGGTGATTGTCCTCACAGCCATGGCCTATGATCGTTATGTGGCCATTTGCAAGCCCTTGCA | |
| TTACTCTTCTATCATGAACAGGAGGCTCTGTGGCATTCTGATGGGGGTAGCCTGGACAGGG | |
| GGCCTCTTGCATTCCATGATACAAATTCTTTTTACTTTCCAGCTTCCCTTTTGTGGCCCCAA | |
| TGTCATCAATCACTTTATGTGTGACTTGTACCCGTTACTGGAGCTTGCCTGCACTGATACTC | |
| ACATCTTTGGCCTCATGGTGGTCATCAACAGTGGGTTTATCTGCATCATAAACTTCTCCTTG | |
| TTGCTTGTCTCCTATGCTGTCATCTTGCTCTCTCTGAGAACACACAGTTCTGAAGGGCGCTG | |
| GAAAGCTCTCTCCACCTGTGGATCTCACATTGCTGTTGTGATTTTGTTCTTTGTCCCATGCA | |
| TATTTGTATATACACGACCTCCATCTGCTTTTTCCCTTGACAAAATGGCGGCAATATTTTAT | |
| ATCATCTTAAATCCCTTGCTCAATCCTTTGATTTACACTTTCAGGAATAAGGAAGTAAAAC | |
| AGGCCATGAGGAGAATATGGAACAGACTGATGGTGGTTTCTGATGAGAAAGAAAATATTA | |
| AACTTTAA | |
| AOLFR91 sequences: | |
| MGNWSTVTEITLIAFPALLEIRISLFVVLVVTYTLTATGNITIISLIWIDHRLQTPMTTFLSNLSFL | (SEQ ID NO:169) |
| DILYTTVITPKLLACLLGEEKTISFAGCMIQTTTTTFLGTVEFILLAVMSFDRYMAICDPLHYTVI | |
| MNSRACLLLVLGCWVGAFLSVLFPTIVVTRLPYCRKEINHFFCDIAPLLQVACINTHLIEKINFLL | |
| SALVILSSLAFTTGSYVYIISTILRIPSTQGRQKAFSTCASHITVVSIAHGSNIFVYVRPNQNSSLD | |
| YDKVAAVLITVVTPLLNPFIYSLRNEKVQEVLRETVNRIMTLIQRKT | |
| ATGGGAAACTGGAGCACTGTGACTGAAATCACCCTAATTGCCTTCCCAGCTCTCCTGGAGA | (SEQ ID NO:170) |
| TTCGAATATGTCTCTTCGTGGTTCTTGTGGTAACTTACACATTAACAGCAACAGGAAACAT | |
| CACCATCATCTCCCTGATATGGATTGATCATCGCCTGCAAACTCCAATGTACTTCTTCCTCA | |
| GTAATTTGTCCTTTCTGGATATCTTATACACCACTGTCATTACCCCAAAGTTGTTGGCCTGC | |
| CTCCTAGGAGAAGAGAAAACCATATCTTTTGCTGGTTGCATGATCCAAACATATTTCTACT | |
| TCTTTCTGGGGACGGTGGAGTTTATCCTCTTGGCGGTGATGTCCTTTGACCGCTACATGGC | |
| TATCTGCGACCCACTGCACTACACGGTCATCATGAACAGCAGGGCCTGCCTTCTGCTGGTT | |
| CTGGGATGCTGGGTGGGAGCCTTCCTGTCTGTGTTGTTTCCAACCATTGTAGTGACAAGGC | |
| TACCTTACTGTAGGAAAGAAATTAATCATTTCTTCTGTGACATTGCCCCTCTTCTTCAGGTG | |
| GCCTGTATAAATACTCACCTCATTGAGAAGATAAACTTTCTCCTCTCTGCCCTTGTCATCCT | |
| GAGCTCCCTGGCATTCACTACTGGGTCCTACGTGTACATAATTTCTACCATCCTGCGTATCC | |
| CCTCCACCCAGGGCCGTCAGAAAGCTTTTTCTACCTGTGCTTCTCACATCACTGTTGTCTCC | |
| ATTGCCCACGGGAGCAACATCTTTGTGTATGTGAGACCCAATCAGAACTCCTCACTGGATT | |
| ATGACAAGGTGGCCGCTGTCCTCATCACAGTGGTGACCCCTCTCCTGAACCCTTTTATCTA | |
| CAGCTTGAGGAATGAGAAGGTACAGGAAGTGTTGAGAGAGACAGTGAACAGAATCATGAC | |
| CTTGATACAAAGGAAAACTTGA | |
| AOLFR92 sequences: | |
| MRNGTVITEFILLGFPVIQGLQTPLFIAIFLTYILTLAGNGLIIATVWAEPRLQWMTTFLCNLSFLE | (SEQ ID NO:171) |
| IWYTTTVIPKLLGTFVVARTVICMSCCLLQAFFHFFVGTTEFLILTIMSFDRYLTICNPLHHPTIM | |
| TSKLCLQLALSSWVVGFTIVFCQTMLLIQLPFCGNNVISHFYCDVGPSLKAACIDTSILELLGVIA | |
| TILVIPGSLLFNMISYIYILSAILRIPSATGHQKTFSTCASHLTVVSLLYGAVLFMYLRPTAHSSFK | |
| INKVVSVLNTILTPLLNIPFIYTIRNKEVKGALRKAMTCPKTGHAK | |
| ATGAGAAATGGCACAGTAATCACAGAATTCATCCTGCTAGGCTTTCCTGTTATCCAAGGCC | (SEQ ID NO:172) |
| TACAAACACCTCTCTTTATTGCAATCTTTCTCACCTACATATTAACCCTTGCAGGCAATGGG | |
| CTTATTATTGCCACTGTGTGGGCTGAGCCCAGGCTACAAATTCCAATGTACTTCTTCCTTTG | |
| TAACTTGTCTTTCTTAGAAATCTGGTACACCACCACAGTCATCCCCAAACTGCTAGGAACC | |
| TTTGTAGTGGCAAGAACAGTAATCTGCATGTCCTGCTGCCTGCTGCAGGCCTTCTTCCACT | |
| TCTTCGTGGGCACCACCGAGTTCTTGATCCTCACTATCATGTCTTTTGACCGCTACCTCACC | |
| ATCTGCAATCCCCTTCACCACCCCACCATCATGACCAGCAAACTCTGCCTGCAGCTGGCCC | |
| TGAGCTCCTGGGTGGTGGGCTTCACCATTGTCTTTTGTCAGACGATGCTGCTCATCCAGTT | |
| GCCATTCTGTGGCAATAATGTTATCAGTCATTTCTACTGTGATGTTGGGCCCAGTTTGAAA | |
| GCCGCCTGCATAGACACCAGCATTTTGGAACTCCTGGGCGTCATAGCAACCATCCTTGTGA | |
| TCCCAGGGTCACTTCTCTTTAATATGATTTCTTATATCTACATTCTGTCCGCAATCCTACGA | |
| ATTCCTTCAGCCACTGGCCACCAAAAGACTTTCTCTACCTGTGCCTCGCACCTGACAGTTGT | |
| CTCCCTGCTCTACGGGGCTGTTCTGTTCATGTACCTAAGACCCACAGCACACTCCTCCTTTA | |
| AGATTAATAAGGTGGTGTCTGTGCTAAATACTATCCTCACCCCCCTTCTGAATCCCTTTATT | |
| TATACTATTAGAAACAAGGAGGTGAAGGGAGCCTTAAGAAAGGCAATGACTTGCCCAAAG | |
| ACTGGTCATGCAAAGTAA | |
| AOLFR93 sequences: | |
| MLMNYSSATEFYLLGFPGSEELHHILFAIFFFFYLVTLMGNTVIIMIVCVDKRLQSPMTTFLGHL | (SEQ ID NO:173) |
| SALEILVTTIIVPVMLWGLLLPGMQTIYLSACVVQLFLYLAVGTTEFALLGAMAVDRYVAVCN | |
| PLRYNIIMNRHTCNFVVLVSWVFGFLFQIWPVYVMFQLTYCKSNVVNNFFCDRGQLLKLSCN | |
| NTLFTEFILFLMAVFVLFGSLIPTIVSNAYIISTILKIPSSSGRRKSFSTCASHFTCVVIGYGSCLFLY | |
| VKPKQTQAADYNWVVSLMVSVVTPFLNPFIFTLRNDKVIEALRDGVKRCCQLFRN | |
| ATGTTGATGAATTACTCTAGTGCCACTGAATTTTATCTCCTTGGCTTCCCTGGCTCTGAAGA | (SEQ ID NO:174) |
| ACTACATCATATCCTTTTTGCTATATTCTTCTTTTTCTACTTGGTGACATTAATGGGAAACA | |
| CAGTCATCATCATGATTGTCTGTGTGGATAAACGTCTGCAGTCCCCCATGTATTTCTTCCTC | |
| GGCCACCTCTCTGCCCTGGAGATCCTGGTCACAACCATAATCGTCCCCGTGATGCTTTGGG | |
| GATTGCTGCTCCCTGGGATGCAGACAATATATTTGTCTGCCTGTGTTGTCCAGCTCTTCTTG | |
| TACCTTGCTGTGGGGACAACAGAGTTCGCATTACTTGGAGCAATGGCTGTGGACCGTTATG | |
| TGGCTGTCTGTAACCCTCTGAGGTACAACATCATTATGAACAGACACACCTGCAACTTTGT | |
| GGTTCTTGTGTCATGGGTGTTTGGGTTTCTTTTTCAAATCTGGCCGGTCTATGTCATGTTTC | |
| AGCTTACTTACTGCAAATCAAATGTGGTGAACAATTTTTTTTGTGACCGAGGGCAATTGCT | |
| CAAACTATCCTGCAATAATACTCTTTTCACGGAGTTTATCCTCTTCTTAATGGCTGTTTTTG | |
| TTCTCTTTGGTTCTTTGATCCCTACAATTGTCTCCAACGCCTACATCATCTCCACCATTCTC | |
| AAGATCCCGTCATCCTCTGGCCGGAGGAAATCCTTCTCCACTTGTGCCTCCCACTTCACCTG | |
| TGTTGTGATTGGCTACGGCAGCTGCTTGTTTCTCTACGTGAAACCCAAGCAAACGCAGGCA | |
| GCTGATTACAATTGGGTAGTTTCCCTGATGGTTTCAGTAGTAACTCCTTTCCTCAATCCTTT | |
| CATCTTCACCCTCCGGAATGATAAAGTCATAGAGGCCCTTCGGGATGGGGTGAAACGCTGC | |
| TGTCAACTATTCAGGAATTAG | |
| AOLFR94 sequences: | |
| METWVNQSYTDGFFLLGIFSHSTADLVLFSVVMAVFTVALCGNVLLTFLIYMDPHLHTPMTTF | (SEQ ID NO:175) |
| LSQLSLMDLMLVCTNVPKMAANFLSGRKSISFVGCGIQIGLFVCLVGSEGLLLGLMAYDRYVA | |
| ISHPLHYPILMNQRVCLQITGSSWAFGIIDGLIQMVVVMNPYCGLRKVNHFFCEMLSLLKLAC | |
| VDTSLFEKVTFACCVFMLLFPFSIIVASYAHILGTVLQMHSAQAWKKALATCSSHLTAVTLFYG | |
| AAMFIYLRPRHYRAPSHDKVASIFYTVLTPMLNPLIYSLRNREVMGALRKGLDRCRIGSQH | |
| ATGGAGACGTGGGTGAACCAGTCCTACACAGATGGCTTCTTCCTCTTAGGCATCTTCTCCC | (SEQ ID NO:176) |
| ACAGTACTGCTGACCTTGTCCTCTTCTCCGTGGTTATGGCGGTCTTCACAGTGGCCCTCTGT | |
| GGGAATGTCCTCCTCATCTTCCTCATCTACATGGACCCTCACCTTCACACCCCCATGTACTT | |
| CTTCCTCAGCCAGCTCTCCCTCATGGACCTCATGTTGGTCTGTACCAATGTGCCAAAGATG | |
| GCAGCCAACTTCCTGTCTGGCAGGAAGTCCATCTCCTTTGTGGGCTGTGGCATACAAATTG | |
| GCCTCTTTGTCTGTCTTGTGGGATCTGAGGGGCTCTTGCTGGGACTCATGGCTTATGACCG | |
| CTATGTGGCCATTAGCCACCCACTTCACTATCCCATCCTCATGAATCAGAGGGTCTGTCTCC | |
| AGATTACTGGGAGCTCCTGGGCCTTTGGGATAATCGATGGCTTGATCCAGATGGTGGTAGT | |
| AATGAATTTCCCCTACTGTGGCTTGAGGAAGGTGAACCATTTCTTCTGTGAGATGCTATCC | |
| TTGTTGAAGCTGGCCTGTGTAGACACATCCCTGTTTGAGAAGGTGATATTTGCTTGCTGTG | |
| TCTTCATGCTTCTCTTCCCATTCTCCATCATCGTGGCCTCCTATGCTCACATTCTAGGGACT | |
| GTGCTGCAAATGCACTCTGCTCAGGCCTGGAAAAAGGCCCTGGCCACCTGCTCCTCCCACC | |
| TGACAGCTGTCACCCTCTTCTATGGGGCAGCCATGTTCATCTACCTGAGGCCTAGGCACTA | |
| CCGGGCCCCCAGCCATGACAAGGTGGCCTCTATCTTCTACACGGTCCTTACTCCCATGCTC | |
| AACCCCCTCATTTACAGCTTGAGGAACAGGGAGGTGATGGGGGCACTGAGGAAGGGGCTG | |
| GACCGCTGCAGGATCGGCAGCCAGCACTGA | |
| AOLFR95 sequences: | |
| MLGSKPRVHLYILPCASQQVSTMGDRGTSNHSEMTDFILAGFRVRPELHILLFLLFLFVYAMILL | (SEQ ID NO:177) |
| GNVGMMTIIMTDPRLNTPMTTFLGNLSFIDLFYSSVIEPKAMINFWSENKSISFAGCVAQLFLFA | |
| LLIVTEGFLLAAMAYDRFIAICNPLLYSVQMSTRLCTQLVAGSTTCGCISSVIQTSMTFTLSFCAS | |
| RAVDHFYCDSRPLQRLSCSDLFIHRMISFSLSCIIILPTIIVIIVSYMYIVSTVLKIHSTEGHKKAFST | |
| CSSHLGVVSVLYGAVFFMYLTPDRFPELSKVASLCYSLVTPMLNPLIYSLRNKDVQEALKKFLE | |
| KKNIIL | |
| ATGCTAGGATCCAAACCAAGAGTTCATTTGTATATTTTGCCCTGTGCCTCTCAACAGGTTTC | (SEQ ID NO:178) |
| TACCATGGGTGACAGGGGAACAAGCAATCACTCAGAAATGACTGACTTCATTCTTGCAGG | |
| CTTCAGGGTACGCCCAGAGCTCCACATTCTCCTCTTCCTGCTATTTTTGTTTGTTTATGCCA | |
| TGATCCTTCTAGGGAATGTTGGGATGATGACCATTATTATGACTGATCCTCGGCTGAACAC | |
| ACCAATGTATTTTTTCCTAGGCAATCTCTCCTTCATTGATCTTTTCTATTCATCTGTTATTGA | |
| ACCCAAGGCTATGATCAACTTCTGGTCTGAAAACAAGTCTATCTCCTTTGCAGGCTGTGTG | |
| GCCCAGCTCTTTCTCTTTGCCCTCCTCATTGTGACTGAGGGATTTCTCCTGGCGGCCATGGC | |
| TTATGACCGCTTTATTGCCATCTGCAACCCTCTGCTCTACTCTGTTCAAATGTCCACACGTC | |
| TGTGTACTCAGTTGGTGGCTGGTTCCTATTTTTGTGGCTGCATTAGCTCAGTTATTCAGACT | |
| AGCATGACATTTACTTTATCTTTTTGCGCTTCTCGGGCTGTTGACCACTTTTACTGTGATTC | |
| TCGCCCACTTCAGAGACTGTCTTGTTCTGATCTCTTTATCCATAGAATGATATCTTTTTCCT | |
| TATCATGTATTATTATCTTGCCTACTATCATAGTCATTATAGTATCTTACATGTATATTGTG | |
| TCCACAGTTCTAAAGATACATTCTACTGAGGGACATAAGAAGGCCTTCTCCACCTGCAGCT | |
| CTCACCTGGGAGTTGTGAGTGTGCTGTATGGTGCTGTCTTTTTTATGTATCTCACTCCTGAC | |
| AGATTTCCTGAGCTGAGTAAAGTGGCATCCTTATGTTACTCCCTAGTCACTCCCATGTTGA | |
| ATCCTTTGATTTACTCTCTGAGGAACAAAGATGTCCAAGAGGCTCTAAAAAAATTTCTAGA | |
| GAAGAAAAATATTATTCTTTGA | |
| AOLFR96 sequences: | |
| MICENHTRVTEFILLGFTNNPEMQVSLFIFFLAIYTVTLLGNFLIVTVTSVDLALQTPMTTFLQN | (SEQ ID NO:179) |
| LSLLEVCFTLVMVPKMLVDLVSPRKIISFVGCGTQMTTFFFFGSSECFLLSMMAYDRFVAICNP | |
| LHYSVIMNRSLCLWMAIGSWMSGVPVSMLQTAWMMALPFCGPNAVDHFFCDGPPVLKLVTV | |
| DTTMYEMQALASTLLFIMFPFCLILVSYTRIIITILRMSSATGRQKAFSTCSSHLIVVSLFYGTASL | |
| TYLRPKSNQSPESKKLVSLSYTVITPMLNPIIYGLRNNEVKGAVKRTITQKVLQLDVF | |
| ATGATCTGTGAAAATCACACCAGAGTCACTGAATTTATTCTTCTTGGTTTTACAAACAACC | (SEQ ID NO:180) |
| CCGAGATGCAAGTTTCCCTCTTTATTTTTTTCCTGGCCATTTATACAGTCACTTTGTTGGGC | |
| AACTTTCTTATTGTCACAGTTACCAGTGTGGATCTCGCACTTCAAACACCCATGTACTTCTT | |
| TCTTCAAAATCTGTCACTTCTTGAAGTATGTTTCACCTTGGTTATGGTGCCAAAAATGCTTG | |
| TAGATCTAGTGTCCCCAAGGAAAATTATCTCTTTTGTGGGCTGTGGTACCCAGATGTACTT | |
| CTTCTTCTTCTTTGGCAGTTCTGAATGTTTCCTTCTCTCCATGATGGCTTATGATCGCTTTGT | |
| GGCCATCTGTAACCCTCTCCATTATTCAGTCATAATGAACAGGTCCCTATGCTTGTGGATG | |
| GCCATAGGCTCTTGGATGTCCGGTGTTCCTGTGTCTATGCTACAGACAGCTTGGATGATGG | |
| CCCTTCCTTTCTGTGGACCAAATGCCGTGGACCACTTTTTCTGTGATGGTCCCCCAGTGTTA | |
| AAACTAGTCACAGTGGATACAACCATGTATGAAATGCAAGCACTTGCCTCCACACTCCTGT | |
| TTATCATGTTTCCCTTTTGTCTCATTTTGGTTTCCTACACCCGCATTATCATAACAATTCTG | |
| AGGATGTCCTCTGGCACTGGCCGCCAGAAGGCATTTTCTACTTGTTCCTCACACCTCATTGT | |
| GGTGTCCCTCTTCTACGGAACAGCCAGTCTGACCTACCTGCGGCCCAAATCAAACCAGTCC | |
| CCTGAGAGCAAGAAGCTAGTGTCATTGTCCTACACTGTCATCACACCTATGCTAAACCCCA | |
| TCATCTACGGCCTGAGGAACAATGAAGTGAAAGGGGCTGTCAAGAGGACAATCACTCAAA | |
| AAGTCTTACAGAAGTTAGATGTGTTTTGA | |
| AOLFR97 sequences: | |
| MTEFHLQSQMPSIRLIFRRLSLGRIKPSQSPRCSTSFMVVPSFSIAEHWRRMKGANLSQGMEFEL | (SEQ ID NO:181) |
| LGLTTDPQLQRLLFVVFLGMYTATLLGNLVMFLLIHVSATLHTPMYSLLKSLSFLDFCYSSTVV | |
| PQTLVNFLAKRKVISTTGCMTQMFFYAGFATSECYLIAAMAYDRYAAICNPLLYSTIMSPEVC | |
| ASLIVGSYSAGFLNSLIHTGCIFSLKFCGAHVVTHFFCDGPPILSLSCVDTSLCEILLFIFAGFNLLS | |
| CTLTILISTTLILNTILKMSSAQGRFKAFSTCASHLTAICLFFGTTLFMYLRPRSSYSLTQDRTVA | |
| VIYTVVIPVLNPLMYSLRNKDVKKALIKVWGRKTME | |
| ATGACAGAGTTTCATCTGCAAAGCCAAATGCCCTCAATAAGACTCATCTTCAGAAGGCTGT | (SEQ ID NO:182) |
| CCTTAGGCAGAATTAAACCCAGTCAGAGCCCCAGGTGTTCAACCTCATTTATGGTGGTGCC | |
| TTCTTTCTCCATCGCAGAGCACTGGAGAAGGATGAAAGGGGCAAACCTGAGCCAAGGGAT | |
| GGAGTTTGAGCTCTTGGGCCTCACCACTGACCCCCAGCTCCAGAGGCTGCTCTTCGTGGTG | |
| TTCCTGGGCATGTACACAGCCACTCTGCTGGGGAACCTGGTCATGTTCCTCCTGATCCATG | |
| TGAGTGCCACCCTGCAGACACCCATGTACTCCCTCCTGAAGAGCCTCTCCTTCTTGGATTTC | |
| TGCTACTCCTCCACGGTTGTGCCCCAGACCCTGGTGAACTTCTTGGCCAAGAGGAAAGTGA | |
| TCTCTTATTTTGGCTGCATGACTCAGATGTTCTTCTATGCGGGTTTTGCCACCAGTGAGTGC | |
| TATCTCATCGCTGCCATGGCCTATGACCGCTATGCCGCTATTTGTAACCCCCTGCTCTACTC | |
| AACCATCATGTCTCCTGAGGTCTGTGCCTCGCTGATTGTGGGCTCCTACAGTGCAGGATTC | |
| CTCAATTCTCTTATCCACACTGGCTGTATCTTTAGTCTGAAATTCTGCGGTGCTCATGTCGT | |
| CACTCACTTCTTCTGTGATGGGCCACCCATCCTGTCCTTGTCTTGTGTAGAGACCTCACTGT | |
| GTGAGATCCTGCTCTTCATTTTTTGCTGGTTTCAACCTTTTGAGCTGCACCCTCACCATCTTG | |
| ATCTCCTACTTCTTAATTCTCAACACCATCCTGAAAATGAGCTCGGCCCAGGGCAGGTTTA | |
| AGGCATTTTCCACCTGTGCATCCCACCTCACTGCCATCTGCCTCTTCTTTGGCACAACACTT | |
| TTTATGTACCTGCGCCCCAGGTCCAGCTACTCCTTGACCCAGGACCGCACAGTTGCTGTCA | |
| TCTACACAGTGGTGATCCCAGTGCTGAACCCCCTCATGTACTCTTTGAGAAACAAGGATGT | |
| GAAGAAAGCTTTAATAAAGGTTTGGGGTAGGAAAACAATGGAATGA | |
| AOLFR98 sequences: | |
| MRGFNKTTVVTQFILVGFSSLGELQLLLFVIFLLLYLTILVANVTIMAVIRFSWTLHTPMYGFLFI | (SEQ ID NO:183) |
| LSFSESCYTFVIIPQLLVHLLSDTKTISFMACATQLFFFLGFACTNCLLIAVMGYDRYVAICHPLR | |
| YTLIINKRLGLELISLSGATGFFIALVATNLICDMRFCGPNRVNHTTCDMAPVIKLACTDTHVKE | |
| LALFSLSILVIMVPFLLILISYGFIVNTILKIPSAEGKKAFVTCASHLTVVFVHYGCASIIYLRPKSK | |
| SASDKDQLVAVTYTVVTPLLNPLVYSLRNKEVKTALKRVLGMPVATKMS | |
| ATGCGAGGTTTCAACAAAACCACTGTGGTTACACAGTTCATCCTGGTGGGTTTCTCCAGCC | (SEQ ID NO:184) |
| TGGGGGAGCTCCAGCTGCTGCTTTTTGTCATCTTTCTTCTCCTATACTTGACAATCCTGGTG | |
| GCCAATGTGACCATCATGGCCGTTATTCGCTTCAGCTGGACTCTCCACACTCCCATGTATG | |
| GCTTTCTATTCATCCTTTCATTTTCTGAGTCCTGCTACACTTTTGTCATCATCCCTCAGCTGC | |
| TGGTCCACCTGCTCTCAGACACCAAGACCATCTCCTTCATGGCCTGTGCCACCCAGCTGTT | |
| CTTTTTCCTTGGCTTTGCTTGCACCAACTGCCTCCTCATTGCTGTGATGGGATATGATCGCT | |
| ATGTAGCAATTTGTCACCCTCTGAGGTACACACTCATCATAAACAAAAGGCTGGGGTTGGA | |
| GTTGATTTCTCTCTCAGGAGCCACAGGTTTCTTTATTGCTTTGGTGGCCACCAACCTCATTT | |
| GTGACATGCGTTTTTGTGGCCCCAACAGGGTTAACCACTATTTCTGTGACATGGCACCTGT | |
| TATCAAGTTAGCCTGCACTGACACCCATGTGAAAGAGCTGGCTTTATTTAGCCTCAGCATC | |
| CTGGTAATTATGGTGCCTTTTCTGTTAATTCTCATATCCTATGGCTTCATAGTTAACACCAT | |
| CCTGAAGATCCCCTCAGCTGAGGGCAAGAAGGCCTTTGTCACCTGTGCCTCACATCTCACT | |
| GTGGTCTTTGTCCACTATGGCTGTGCCTCTATCATCTATCTGCGGCCCAAGTCCAAGTCTGC | |
| CTCAGACAAGGATCAGTTGGTGGCAGTGACCTACACAGTGGTTACTCCCTTACTTAATCCT | |
| CTTGTCTACAGTCTGAGGAACAAAGAGGTAAAAACTGCATTGAAAAGAGTTCTTGGAATG | |
| CCTGTGGCAACCAAGATGAGCTAA | |
| AOLFR99 sequences: | |
| MERVNETVVREVIFLGFSSLARLQQLLFVIFLLLYLFTLGTNAIIISTIVLDRALHIPMTTFLAILSC | (SEQ ID NO:185) |
| SEICYTFIIVPKMLVDLLSQKKTISFLGCAIQMFSFLFLGCSHSFLLAVMGYDRYIAICNPLRYSV | |
| LMGHGVCMGLVAAACACGFTVAQIITSLVFHLPFYSSNQLHHFFCDIAPVLKLASHHNHFSQIV | |
| IFMLCTLVLAIPLLLILVSYVHILSAILQFPSTLGRCKAFSTCVSHLIIVTVHYGCASFIYLRPQSNY | |
| SSSQDALISVSYTIITPLFNPMIYSLRNKEFKSALCKIVRRTISLL | |
| ATGGAGCGGGTCAATGAGACTGTGGTGAGAGAGGTCATCTTCCTCGGCTTCTCATCCCTGG | (SEQ ID NO:186) |
| CCAGGCTGCAGCAGCTGCTCTTTGTTATCTTCCTGCTGCTCTACCTGTTCACTCTGGGCACC | |
| AATGCAATCATCATTTCCACCATTGTCCTGGACAGGGCCCTTCATATCCCCATGTACTTCTT | |
| CCTTGCCATCCTCTCTTGCTCTGAGATTTGCTACACCTTCATCATTGTACCCAAGATGCTGG | |
| TTGACCTGCTGTCCCAGAAGAAGACCATTTCTTTCCTGGGCTGTGCCATCCAAATGTTTTCC | |
| TTCCTCTTCCTTGGCTGCTCTCACTCCTTTCTGCTGGCAGTCATGGGTTATGATCGTTACAT | |
| AGCCATCTGTAACCCACTGCGCTACTCAGTGCTAATGGGACATGGGGTGTGTATGGGACTA | |
| GTGGCTGCTGCCTGTGCCTGTGGCTTCACTGTTGCACAGATCATCACATCCTTGGTATTTCA | |
| CCTGCCTTTTTATTCCTCCAATCAACTACATCACTTCTTCTGTGACATTGCTCCTGTCCTCA | |
| AGCTGGCATCTCACCATAACCACTTTAGTCAGATTGTCATCTTCATGCTCTGTACATTGGTC | |
| CTGGCTATCCCCTTATTGTTGATCTTGGTGTCCTATGTTCACATCCTCTCTGCCATACTTCA | |
| GTTTCCTTCCACACTGGGTAGGTGCAAAGCTTTTTCTACCTGTGTATCTCACCTCATTATTG | |
| TCACTGTCCACTATGGCTGTGCCTCCTTTATCTACTTAAGGCCTCAGTCCAACTACTCCTCA | |
| AGCCAGGATGCTCTAATATCAGTATCCTACACTATTATAACTCCATTGTTCAACCCAATGA | |
| TTTATAGCTTGAGAAATAAAGAGTTCAAATCAGCTCTTTGTAAAATTGTGAGAAGAACAAT | |
| TTCCCTGTTGTAA | |
| AOLFR101 sequences: | |
| MDTGNSQVAEFIILGFPHLQGVQIYLFLLLLLIYLMTVLGNLLIFLVVCLDSRLHTPMYHFVSI | (SEQ ID NO:187) |
| LSFSELGYTAATIPKMLANLLSEKKTISFSGCLLQITTFHSLGATECYLLTAMAYDRYLAICRPL | |
| HYPTLMTPTLCAEIAIGCWLGGLAGPVVEISLISRLPFCGPNRIQHVFCDFPPVLSLACTDTSINV | |
| LVDFVINSCKILATFLLILCSYVQIICTVLRIPSAAGKRKAISTCASHFTVVLIFYGSILSMYVQLK | |
| KSYSLDYDQALAVVYSVLTPFLNPFIYSLRNKEIKEAVRRQLKRIGILA | |
| ATGGACACAGGGAACTGGAGCCAGGTAGCAGAATTCATCATCTTGGGCTTCCCCCATCTCC | (SEQ ID NO:188) |
| AGGGTGTCCAGATTTATCTCTTCCTCTTGTTGGTTCTCATTTACCTCATGACTGTGTTGGGA | |
| AACCTGCTGATATTCCTGGTGGTCTGCCTGGACTCCCGGCTTCACACACCCATGTACCACT | |
| TTGTCAGCATTCTCTCCTTCTCAGAGCTTGGCTATACAGCTGCCACCATCCCTAAGATGCTG | |
| GCAAACTTGCTCAGTGAGAAAAAGACCATTTCATTCTCTGGGTGTCTCCTGCAGATCTATT | |
| TCTTTCACTCCCTTGGAGCGACTGAGTGCTATCTCCTGACAGCTATGGCCTACGATAGGTA | |
| TTTAGCCATCTGCCGGCCCCTCCACTACCCAACCCTCATGACCCCAACACTTTGTGCAGAG | |
| ATTGCCATTGGCTGTTGGTTGGGAGGCTTGGCTGGGCCAGTAGTTGAAATTTCCTTGATTT | |
| CACGCCTCCCATTCTGTGGCCCCAATCGCATTCAGCACGTCTTTTGTGACTTCCCTCCTGTG | |
| CTGAGTTTGGCTTGCACTGATACGTCTATAAATGTCCTAGTAGATTTTGTTATAAATTCCTG | |
| CAAGATCCTAGCCACCTTCCTGCTGATCCTCTGCTCCTATGTGCAGATCATCTGCACAGTGC | |
| TCAGAATTCCCTCAGCTGCCGGCAAGAGGAAGGCCATCTCCACGTGTGCCTCCCACTTCAC | |
| TGTGGTTCTCATCTTCTATGGGAGCATCCTTTCCATGTATGTGCAGCTGAAGAAGAGCTAC | |
| TCACTGGACTATGACCAGGCCCTGGCAGTGGTCTACTCAGTGCTCACACCCTTCCTCAACC | |
| CCTTCATCTACAGCTTGCGCAACAAGGAGATCAAGGAGGCTGTGAGGAGGCAGCTAAAGA | |
| GAATTGGGATATTGGCATGA | |
| AOLFR102 sequences: | |
| MPVGKLVFNQSEPTEFVFRAFTTATEFQVLLFLLFLLLYLMILCGNTAIIWVVCTHSTLRTPMTT | (SEQ ID NO:189) |
| FLSNLSFLELCYTTVVVPLMLSNILGAQKPISLAGCGAQMFFFVTLGSTDCFLLAIMAYDRYVAI | |
| CHPLHYTLIMTRELCTQMLGGALGLALFPSLQLTALIFTLPFCGHHQEINHFLCDVPPVLRLACA | |
| DIRVHQAVLYVVSILVLTIPFLLICVSYVFITCAILSIRSAEGRRRAFSTCSFHLTVVLLQYGCCSL | |
| VYLRPRSSTSEDEDSQIALVYTFVTPLLNPLLYSLRNKDVKGALRSAIIRKAASDAN | |
| ATGCCTGTGGGGAAACTTGTCTTCAACCAGTCTGAGCCCACTGAGTTTGTGTTCCGTGCGT | (SEQ ID NO:190) |
| TCACCACAGCCACTGAATTCCAGGTTCTTCTCTTCCTTCTCTTCCTCCTCCTCTACTTGATG | |
| ATCCTCTGTGGCAACACAGCCATCATCTGGGTGGTGTGCACACACAGCACCCTCCGCACCC | |
| CGATGTATTTCTTCCTGTCCAACCTGTCTTTCCTGGAACTCTGCTACACCACCGTGGTAGTA | |
| CCCTTGATGCTTTCCAACATTTTGGGGGCCCAGAAGCCCATTTCGTTGGCTGGATGTGGGG | |
| CCCAAATGTTCTTCTTTGTCACCCTCGGCAGCACGGACTGTTTCCTCTTGGCGATCATGGCC | |
| TATGACCGCTATGTGGCTATCTGCCACCCGCTGCACTACACCCTCATCATGACCCGCGAGC | |
| TGTGCACGCAGATGCTGGGTGGGGCCCTGGGCCTGGCCCTCTTCCCCTCCCTGCAGCTCAC | |
| CGCCTTAATCTTCACCCTGCCCTTTTGCGGCCACCACCAGGAAATCAACCACTTCCTCTGCG | |
| ATGTGCCTCCCGTCCTGCGCCTGGCCTGCGCTGACATCCGCGTGCACCAGGCTGTGCTCTA | |
| TGTCGTGAGCATCCTCGTGCTGACCATCCCCTTCCTGCTCATCTGCGTCTCCTACGTGTTCA | |
| TCACCTGTGCCATCCTGAGCATCCGTTCTGCCGAGGGCCGCCGCCGGGCCTTCTCCACCTG | |
| CTCCTTCCACCTCACCGTGGTCCTGCTGCAGTATGGCTGCTGCAGCCTCGTGTACCTGCGTC | |
| CTCGGTCCAGCACCTCAGAGGATGAGGACAGCCAAATCGCGTTGGTCTACACCTTTGTCAC | |
| CCCCTTACTCAACCCTTTGCTTTACAGCCTTAGGAACAAGGATGTCAAAGGTGCTCTGAGG | |
| AGTGCCATTATCCGTAAAGCAGCCTCTGACGCCAACTGA | |
| AOLFR103 sequences: | |
| MAEMNLTLVTEFLLIAFTEYPEWALPLFLLLLFMYLITVLGNLEMIILILMDHQLHAPMTTLLSH | (SEQ ID NO:191) |
| LAFMDVCYSSITVPQMLAVLLEHGAALSYTRCAAQFFLFTFFGSIDCYLLALMAYDRYLAVCQ | |
| PLLYVTILTQQARLSLVAGAYVAGLISALVRTVSAFTLSFCGTSEIDFIFCDLPPLLKLTCGESYT | |
| QEVLIIMFAIFVIPASMVVILVSYLFIIVAIMGIPAGSQAKTFSTCTSHLTAVSLFFGTLIFMYLRG | |
| NSDQSSEKNRVVSVLYTEVIPMLNPLIYSLRNKEVKEALRKILNRAKLS | |
| ATGGCAGAGATGAACCTCACCTTGGTGACCGAGTTCCTCCTTATTGCATTCACTGAATATC | (SEQ ID NO:192) |
| CTGAATGGGCACTCCCTCTCTTCCTCTTGTTATTATTTATGTATCTCATCACCGTATTGGGG | |
| AACTTAGAGATGATTATTCTGATCCTCATGGATCACCAGCTCCACGCTCCAATGTATTTCCT | |
| TCTGAGTCACCTCGCTTTCATGGACGTCTGCTACTCATCTATCACTGTCCCCCAGATGCTGG | |
| CAGTGCTGCTGGAGCATGGGGCAGCTTTATCTTACACACGCTGTGCTGCTCAGTTCTTTCT | |
| GTTCACCTTCTTTGGTTCCATCGACTGCTACCTCTTGGCCCTCATGGCCTATGACCGCTACT | |
| TGGCTGTGTGCCAGCCCCTGCTTTATGTCACCATCCTGACACAGCAGGCCCGCTTGAGTCT | |
| TGTGGCTGGGGCTTACGTTGCTGGTCTCATCAGTGCCTTGGTGCGGACAGTCTCAGCCTTC | |
| ACTCTCTCCTTCTGTGGAACCAGTGAGATTGACTTTATTTTCTGTGACCTCCCTCCTCTGTT | |
| AAAGTTGACCTGTGGGGAGAGCTACACTCAAGAAGTGCTGATTATTATGTTTGCCATTTTT | |
| GTCATCCCTGCTTCCATGGTGGTGATCTTGGTGTCCTACCTGTTTATCATCGTGGCCATCAT | |
| GGGGATCCCTGCTGGAAGCCAGGCCAAGACCTTCTCCACCTGCACCTCCCACCTCACTGCT | |
| GTGTCACTCTTCTTTGGTACCCTCATCTTCATGTACTTGAGAGGTAACTCAGATCAGTCTTC | |
| GGAGAAGAATCGGGTAGTGTCTGTGCTTTACACAGAGGTCATCCCCATGTTGAATCCCCTC | |
| ATCTACAGCCTGAGGAACAAGGAAGTGAAGGAGGCCCTGAGAAAAATTCTCAATAGAGCC | |
| AAGTTGTCCTAA | |
| AOLFR105 sequences: | |
| MQGLNHTSVSEFILVGFSAFPHLQLMLFLLFLLMYLFTLLGNLLIMATVWSERSLHMPMYLFLC | (SEQ ID NO:193) |
| ALSITEILYTVAIIPRMLADLLSTQRSIAFLACASQMFFSFSFGFTHSFLLTVMGYDRYVAICHPL | |
| RYNVLMSLRGCTCRVGCSWAGGLVMGMVVTSAIFHLAFCGHKEIHHFFCHVPPLLKLACGDD | |
| VLVVAKGVGLVCITALLGCFLLILLSYAFIVAAILKIPSAERNKAFSTCASHLTVVVVGFAS | |
| VIYLKPKGPQSPEGDTLMGITYTVLTPFLSPIIFSLRNKELKVAMKKTCFTKLFPQNC | |
| ATGCAGGGGCTAAACCACACCTCCGTGTCTGAATTCATCCTCGTTGGCTTCTCTGCCTTCCC | (SEQ ID NO:194) |
| CCACCTCCAGCTGATGCTCTTCCTGCTGTTCCTGCTGATGTACCTGTTCACGCTGCTGGGCA | |
| ACCTGCTCATCATGGCCACTGTCTGGAGCGAGCGCAGCCTCCACATGCCCATGTACCTCTT | |
| CCTGTGTGCCCTCTCCATCACCGAGATCCTCTACACCGTGGCCATCATCCCGCGCATGCTG | |
| GCCGACCTGCTGTCCACCCAGCGCTCCATCGCCTTCCTGGCCTGTGCCAGTCAGATGTTCTT | |
| CTCCTTCAGCTTCGGCTTCACCCACTCCTTCCTGCTCACTGTCATGGGCTACGACCGCTACG | |
| TGGCCATCTGCCACCCCCTGCGTTACAACGTGCTCATGAGCCTGCGGGGCTGCACCTGCCG | |
| GGTGGGCTGCTCCTGGGCTGGTGGCTTGGTCATGGGGATGGTGGTGACCTCGGCCATTTTC | |
| CACCTCGCCTTCTGTGGACACAAGGAGATCCACCATTTCTTCTGCCACGTGCCACCTCTGTT | |
| GAAGTTGGCCTGTGGAGATGATGTGCTGGTGGTGGCCAAAGGCGTGGGCTTGGTGTGTAT | |
| CACGGCCCTGCTGGGCTGTTTTCTCCTCATCCTCCTCTCCTATGCCTTCATCGTGGCCGCCA | |
| TCTTGAAGATCCCTTCTGCTGAAGGTCGGAACAAGGCCTTCTCCACCTGTGCCTCTCACCT | |
| CACTGTGGTGGTCGTGCACTATGGCTTTGCCTCCGTCATTTACCTGAAGCCGAAAGGTCCC | |
| CAGTCTCCGGAAGGAGACACCTTGATGGGCATCACCTACACGGTCCTCACACCCTTCCTCA | |
| GCCCCATCATCTTCAGCCTCAGGAACAAGGAGCTGAAGGTCGCCATGAAGAAGACTTGCTT | |
| CACCAAACTCTTTCCACAGAACTGCTGA | |
| AOLFR106 sequences: | |
| METANYTKVTEFVLTGLSQTPEVQLVLFVIFLSFYLFILPGNILIICTISLDPHLTSPMTTLLANLA | (SEQ ID NO:195) |
| FLDIWYSSITAPEMLIDFFVERKIISFDGCIAQLFFLHFAGASEMFLLTVMAFDLYTAICRPLHYA | |
| TIMNQRLCCILVALSWRGGFIHSIIQVALIVRLPFCGPNELDSTTCDITQVVRIACANTFPEELVM | |
| ICSSGLISVVCLIALLMSYAFLLALFKKLSGSGENTNRAMSTCYSHITIVVLMFGPSIYIYARPFD | |
| SFSLDKVVSVFNTLIFPLRNPIIYTLRNKEVKAAMRKLVTKYILCKEK | |
| ATGGAAACTGCAAATTACACCAAGGTGACAGAATTTGTTCTCACTGGCCTATCCCAGACTC | (SEQ ID NO:196) |
| CAGAGGTCCAACTAGTCCTATTTGTTATATTTCTATCCTTCTATTTGTTCATCCTACCAGGA | |
| AATATCCTTATCATTTGCACCATCAGTCTAGACCCTCATCTGACCTCTCCTATGTATTTCCT | |
| GTTGGCTAATCTGGCCTTCCTTGATATTTGGTACTCTTCCATTACAGCCCCTGAAATGCTCA | |
| TAGACTTCTTTGTGGAGAGGAAGATAATTTCTTTTGATGGATGCATTGCACAGCTCTTCTT | |
| CTTACACTTTGCTGGGGCTTCGGAGATGTTCTTGCTCACAGTGATGGCCTTTGACCTCTACA | |
| CTGCTATCTGCCGACCCCTCCACTATGCTACCATCATGAATCAACGTCTCTGCTGTATCCTG | |
| GTGGCTCTCTCCTGGAGGGGGGGCTTCATTCATTCTATCATACAGGTGGCTCTCATTGTTC | |
| GACTTCCTTTCTGTGGGCCCAATGAGTTAGACAGTTACTTCTGTGACATCACACAGGTTGT | |
| CCGGATTGCCTGTGCCAACACCTTCCCAGAGGAGTTAGTGATGATCTGTAGTAGTGGTCTG | |
| ATCTCTGTGGTGTGTTTGATTGCTCTGTTAATGTCCTATGCCTTCCTTCTGGCCTTGTTCAA | |
| GAAACTTTCAGGCTCAGGTGAGAATACCAACAGGGCCATGTCCACCTGCTATTCCCACATT | |
| ACCATTGTGGTGCTAATGTTTGGGCCATCCATCTACATTTATGCTCGCCCATTTGACTCGTT | |
| TTCCCTAGATAAAGTGGTGTCTGTGTTCAATACTTTAATATTCCCTTTACGTAATCCCATTA | |
| TTTACACATTGAGAAACAAGGAAGTAAAGGCAGCCATGAGGAAGTTGGTCACCAAATATA | |
| TTTTGTGTAAAGAGAAGTGA | |
| AOLFR107 sequences: | |
| MELWFTLGSGFILVGILNDSGSPELLCATITILYLLALISNGLLLLAITMEARLHMPMYLLLGQ | (SEQ ID NO:197) |
| LSLMDLLFTSVVTPKALADFLRRENTISFGGCALQMFLALTMGGAEDLLLAFMEYDRYVAICH | |
| PLTYMTLMSSRACWLMVATSWILASLSALIYTVYTMHYPFCRAQEIRHLLCEIPHLLKVACAD | |
| TSRYELMVYVMGVTFLIPSLAAILASYTQILLTVLHMPSNEGRKKALVTCSSHLTVVGMFYGA | |
| ATFMYVLPSSFHSTRQDNIISVFYTIVTPALNPLIYSLRNKEVMRALRRVLGKYMLPAHSTL | |
| ATGGAGCTCTGGAACTTCACCTTGGGAAGTGGCTTCATTTTGGTGGGGATTCTGAATGACA | (SEQ ID NO:198) |
| GTGGGTCTCCTGAACTGCTCTGTGCTACAATTACAATCCTATACTTGTTGGCCCTGATCAG | |
| CAATGGCCTACTGCTCCTGGCTATCACCATGGAAGCCCGGCTCCACATGCCCATGTACCTC | |
| CTGCTTGGGCAGCTCTCTCTCATGGACCTCCTGTTCACATCTGTTGTCACTCCCAAGGCCCT | |
| TGCGGACTTTCTGCGCAGAGAAAACACCATCTCCTTTGGAGGCTGTGCCCTTCAGATGTTC | |
| CTGGCACTGACAATGGGTGGTGCTGAGGACCTCCTACTGGCCTTCATGGCCTATGACAGGT | |
| ATGTGGCCATTTGTCATCCTCTGACATACATGACCCTCATGAGCTCAAGAGCCTGCTGGCT | |
| CATGGTGGCCACGTCCTGGATCCTGGCATCCCTAAGTGCCCTAATATATACCGTGTATACC | |
| ATGCACTATCCCTTCTGCAGGGCCCAGGAGATCAGGCATCTTCTCTGTGAGATCCCACACT | |
| TGCTGAAGGTGGCCTGTGCTGATACCTCCAGATATGAGCTCATGGTATATGTGATGGGTGT | |
| GACCTTCCTGATTCCCTCTCTTGCTGCTATACTGGCCTCCTATACACAAATTCTACTCACTG | |
| TGCTCCATATGCCATCAAATGAGGGGAGGAAGAAAGCCCTTGTCACCTGCTCTTCCCACCT | |
| GACTGTGGTTGGGATGTTCTATGGAGCTGCCACATTCATGTATGTCTTGCCCAGTTCCTTCC | |
| ACAGCACCAGACAAGACAACATCATCTCTGTTTTCTACACAATTGTCACTCCAGCCCTGAA | |
| TCCACTCATCTACAGCCTGAGGAATAAGGAGGTCATGCGGGCCTTGAGGAGGGTCCTGGG | |
| AAAATACATGCTGCCAGCACACTCCACGCTCTAG | |
| AOLFR108 sequences: | |
| MCSFFLCQTGKQAKISMGEENQTFVSKFIFLGLSQDLQTQILLFILFLIIYLLTVLGNQLIIILIFLD | (SEQ ID NO:199) |
| SRLHTPMTTFLRNLSFADLCFSTSIVPQVLVHFLVKRKTISFYGCMTQIIVFLLVGCTECALLAV | |
| MSYDRYVAVCKPLYYSTIMTQRVCLWLSFRSWASGALVSLVDTSFTFHLPYWGQNIINHTTCE | |
| PPALLKLASIDTYSTEMAIFSMGVVILLAPVSLILGSYWNIISTVIQMQSGEGRLKAFSTCGSHLI | |
| VVVLFYGSGIFTYMRPNSKTTKELDKMISVFYTAVTPMLNPIIYSLRNKDVKGALRKLVGRKC | |
| FSHRQ | |
| ATGTGTTCTTTTTTCTTGTGCCAAACAGGTAAACAGGCAAAAATATCAATGGGAGAAGAAA | (SEQ ID NO:200) |
| ACCAAACCTTTGTGTCCAAGTTTATCTTCCTGGGTCTTTCACAGGACTTGCAGACCCAGAT | |
| CCTGCTATTTATCCTTTTCCTCATCATTTATCTGCTGACCGTGCTTGGAAACCAGCTCATCA | |
| TCATTCTCATCTTCCTGGATTCTCGCCTTCACACTCCCATGTATTTTTTTCTTAGAAATCTCT | |
| CCTTTGCAGATCTCTGTTTCTCTACTAGCATTGTCCCTCAAGTGTTGGTTCACTTCTTGGTA | |
| AAGAGGAAAACCATTTCTTTTTATGGGTGTATGACACAGATAATTGTCTTTCTTCTGGTTG | |
| GGTGTACAGAGTGTGCGCTGCTGGCAGTGATGTCCTATGACCGGTATGTGGCTGTCTGCAA | |
| GCCCCTGTACTACTCTACCATCATGACACAACGGGTGTGTCTCTGGCTGTCCTTCAGGTCCT | |
| GGGCCAGTGGGGCACTAGTGTCTTTAGTAGATACCAGCTTTACTTTCCATCTTCCCTACTG | |
| GGGACAGAATATAATCAATCACTACTTTTGTGAACCTCCTGCCCTCCTGAAGCTGGCTTCC | |
| ATAGACACTTACAGCACAGAAATGGCCATCTTTTCAATGGGCGTGGTAATCCTCCTGGCCC | |
| CTGTCTCCCTGATTCTTGGTTCTTATTGGAATATTATCTCCACTGTTATCCAGATGCAGTCT | |
| GGGGAAGGGAGACTCAAGGCTTTTTCCACCTGTGGCTCCCATCTTATTGTTGTTGTCCTCTT | |
| CTATGGGTCAGGAATATTCACCTACATGCGACCAAACTCCAAGACTACAAAAGAACTGGA | |
| TAAAATGATATCTGTGTTCTATACAGCGGTGACTCCAATGTTGAACCCCATAATTTATAGC | |
| TTGAGGAACAAAGATGTCAAAGGGGCTCTCAGGAAACTAGTTGGGAGAAAGTGCTTCTCT | |
| CATAGGCAGTGA | |
| AOLFR109 sequences: | |
| MLRNGSIVTEFILVGFQQSSTSTRALLFALFLALYSLTMAMNGLIIFITSWTDPKLNSPMTTFLG | (SEQ ID NO:201) |
| HLSLLDVCFITTTIPQMLIHLVVRDHIVSFVCCMTQMTTVFCVGVAECILLAFMAYDRYVAICY | |
| PLNYVPIISQKVCVRLVGTAWFFGLINGIFLEYISFREPFRRDNHIESFFCEAPIVIGLSCGDPQFSL | |
| WAIFADAIVVILSPMVLTVTSYVHILATILSKASSSGRGKTFSTCASHLTVVIFLYTSAMFSYMN | |
| PHSTHGPDKDKPFSLLYTIITPMCNPIIYSFRNKEIKEAMVRALGRTRLAQPQSV | |
| ATGCTAAGGAATGGCAGCATAGTGACGGAATTTATCCTCGTGGGCTTTCAGCAGAGCTCCA | (SEQ ID NO:202) |
| CTTCCACACGAGCATTGCTCTTTGCCCTCTTCTTGGCCCTCTACAGCCTCACCATGGCCATG | |
| AATGGCCTCATCATCTTTATCACCTCCTGGACAGACCCCAAGCTCAACAGCCCCATGTACT | |
| TCTTCCTCGGCCATCTGTCTCTCCTGGATGTCTGCTTCATCACCACTACCATCCCACAGATG | |
| TTGATCCACCTCGTGGTCAGGGACCACATTGTCTCCTTTGTATGTTGCATGACCCAGATGT | |
| ACTTTGTCTTCTGTGTTGGTGTGGCCGAGTGCATCCTCTTGGCTTTCATGGCCTATGACCGT | |
| TATGTTGCTATCTGCTACCCACTTAACTATGTCCCGATCATAAGCCAGAAGGTCTGTGTCA | |
| GGCTTGTGGGAACTGCCTGGTTCTTTGGGCTGATCAATGGCATCTTTCTCGAGTATATTTC | |
| ATTCCGAGAGCCCTTCCGCAGAGACAACCACATAGAAAGCTTCTTCTGTGAGGCCCCCATA | |
| GTGATTGGCCTCTCTTGTGGGGACCCTCAGTTTAGTCTGTGGGCAATCTTTGCCGATGCCA | |
| TCGTGGTAATTCTCAGCCCCATGGTGCTCACTGTCACTTCCTATGTGCACATCCTGGCCACC | |
| ATCCTCAGCAAAGCCTCCTCCTCAGGTCGGGGGAAGACTTTCTCTACTTGTGCCTCTCACC | |
| TGACTGTGGTCATCTTTCTCTACACTTCAGCTATGTTCTCTTACATGAACCCCCACAGCACA | |
| CATGGGCCTGACAAAGACAAACCTTTCTCCCTCCTGTACACCATCATTACCCCCATGTGCA | |
| ACCCCATCATTTATAGTTTCCGCAACAAGGAAATTAAGGAGGCCATGGTGAGGGCACTTG | |
| GAAGAACCAGGCTGGCCCAGCCACAGTCTGTCTAG | |
| AOLFR110 sequences: | |
| MKIANNTVVTEFILLGLTQSQDIQLLVFVLILIFYLIILPGNFLIIFTIRSDPGLTAPLYLFLGNLAFL | (SEQ ID NO:203) |
| DASYSFIVAPRMLVDFLSEKKVISYRGCITQLFFLHFLGGGEGLLLVVMAFDRYIAICRPLHCST | |
| VMNPRACYAMMLALWLGGFVHSIIQVVLILRLPFCGPNQLDNFFCDVRQVIKLACTDMFVVEL | |
| LMVFNSGLMTLLCFLGLLASYAVILCHVRRAASEGKNKAMSTCTTRVIIILLMFGPAIFIYMCPF | |
| RALPADKMVSLFHTVIFPLMNPMIYTLRNQEVKTSMKRLLSRHVVCQVDFIIRN | |
| ATGAAGATAGCAAACAACACAGTAGTGACAGAATTTATCCTCCTTGGTCTGACTCAGTCTC | (SEQ ID NO:204) |
| AAGATATTCAGCTCTTGGTCTTTGTGCTGATCTTAATTTTCTACCTTATCATCCTCCCTGGA | |
| AATTTTCTCATTATTTTCACCATAAGGTCAGACCCTGGGCTCACAGCCCCCCTCTATTTATT | |
| TCTGGGCAACTTGGCCTTCCTGGATGCATCCTACTCCTTCATTGTGGCTCCCAGGATGTTGG | |
| TGGACTTCCTCTCTGAGAAAAAGGTAATCTCCTACAGAGGCTGCATCACTCAGCTCTTTTT | |
| CTTGCACTTCCTTGGAGGAGGGGAGGGATTACTCCTTGTTGTGATGGCCTTTGACCGCTAC | |
| ATCGCCATCTGCCGGCCTCTGCACTGTTCAACTGTCATGAACCCTAGAGCCTGCTATGCAA | |
| TGATGTTGGCTCTGTGGCTTGGGGGTTTTGTCCACTCCATTATCCAGGTGGTCCTCATCCTC | |
| CGCTTGCCTTTTTGTGGCCCAAACCAGCTGGACAACTTCTTCTGTGATGTCCGACAGGTCA | |
| TCAAGCTTGCTTGCACCGACATGTTTGTGGTGGAGCTTCTAATGGTCTTCAACAGTGGCCT | |
| GATGACACTCCTGTGCTTTCTGGGGCTTCTGGCTTCCTATGCAGTCATCCTCTGCCATGTTC | |
| GTAGGGCAGCTTCTGAAGGGAAGAACAAGGCCATGTCCACGTGCACCACTCGTGTCATTA | |
| TTATACTTCTTATGTTTGGACCTGCTATCTTCATCTACATGTGCCCTTTCAGGGCCTTACCA | |
| GCTGACAAGATGGTTTCTCTCTTTCACACAGTGATCTTTCCATTGATGAATCCTATGATTTA | |
| TACCCTTCGCAACCAGGAAGTGAAAACTTCCATGAAGAGGTTATTGAGTCGACATGTAGTC | |
| TGTCAAGTGGATTTTATAATAAGAAACTGA | |
| AOLFR111 sequences: | |
| MCYIYLIFKEWTLIFTTSLLLFLQITPAIMANLTIVTEFILMGFSTNKNMCILHSILFLLIYLCALM | (SEQ ID NO:205) |
| GNVLIIMITTLDHHLHTPVTTFLKNLSFLDLCLISVTAPKSIANSLIHNNSISFLGCVSQVFLLLSS | |
| ASAELLLLTVMSFDRYTAICHPLHYDVIMDRSTCVQRATVSWLYGGLIAVMHTAGTFSLSYCG | |
| SNMVHQFFCDIPQLLAISCSENLIREIALILLNVVLDFCCFIVIIITYVHVFSTVKKIPSTEGQSKAY | |
| SICLPHLLVVLFLSTGFIAYLKPASESPSILDAVISVFYTMLPPTFNPIIYSLRNKAIKVALGMLIKG | |
| KLTKK | |
| ATGTGTTATATATATTTAATATTTAAAGAGTGGACATTGATATTTTACTTCAGTCTTCTCCT | (SEQ ID NO:206) |
| TTTCCTGCAGATTACTCCTGCAATAATGGCAAATCTCACAATCGTGACTGAATTTATCCTTA | |
| TGGGGTTTTCTACCAATAAAAATATGTGCATTTTGCATTCGATTCTCTTCTTGTTGATTTAT | |
| TTGTGTGCCCTGATGGGGAATGTCCTCATTATCATGATCACAACTTTGGACCATCATCTCC | |
| ACACCCCCGTGTATTTCTTCTTGAAGAATCTATCTTTCTTGGATCTCTGCCTTATTTCAGTC | |
| ACGGCTCCCAAATCTATCGCCAATTCTTTGATACACAACAACTCCATTTCATTCCTTGGCTG | |
| TGTTTCCCAGGTCTTTTTGTTGCTTTCTTCAGCATCTGCAGAGCTGCTCCTCCTCACGGTGA | |
| TGTCCTTTGACCGCTATACTGCTATATGTCACCCTCTGCACTATGATGTCATCATGGACAGG | |
| AGCACCTGTGTCCAAAGAGCCACTGTGTCTTGGCTGTATGGGGGTCTGATTGCTGTGATGC | |
| ACACAGCTGGCACCTTCTCCTTATCCTACTGTGGGTCCAACATGGTCCATCAGTTCTTCTGT | |
| GACATTCCCCAGTTATTAGCTATTTCTTGCTCAGAAAATTTAATAAGAGAAATTGCACTCA | |
| TCCTTATTAATGTAGTTTTGGATTTCTGCTGTTTTATTGTCATCATCATTACCTATGTCCAC | |
| GTCTTCTCTACAGTCAAGAAGATCCCTTCCACAGAAGGCCAGTCAAAAGCCTACTCTATTT | |
| GCCTTCCACACTTGCTGGTTGTGTTATTTCTTTCCACTGGATTCATTGCTTATCTGAAGCCA | |
| GCTTCAGAGTCTCCTTCTATTTTGGATGCTGTAATTTCTGTGTTCTACACTATGCTGCCCCC | |
| AACCTTTAATCCCATTATATACAGTTTGAGAAACAAGGCCATAAAGGTGGCTCTGGGGATG | |
| TTGATAAAGGGAAAGCTCACCAAAAAGTAA | |
| AOLFR113 sequences: | |
| MKFWHGFSSHLNPMFSSFLLYLSLPWINTTIQAWLNLCSLALPVWAMSGAGFLSCCYWHTCSP | (SEQ ID NO:207) |
| SVVTCSSSQSSDWMQLCTHLCTTLSVFFPSWSCGIQLPLSLRCCLIFSVRRKPFLLQDASFRPTSS | |
| TPWGACECYLLTAMAYDRYLAICRPLHYPIIMTTTLCAKMAAACWTCGFLCPISEVILASQLPF | |
| CAYNEIQHIFCDFPPLLSLACKDTSANILVDFAINAFIILITFFFIMISYARIIGAVLKIKTASGRKK | |
| AFSTCASHLAVVLIFFGSIIFMYVRLKKSYSLTLDRTLAIVYSVLTPMVNPIIYSLRNKEIIKAIKR | |
| TIFQKGDKASLAHL | |
| ATGTGTCAACAAATCTTACGGGATTGCATTCTTCTCATACATCATTTGTGCATTAACAGGA | (SEQ ID NO:208) |
| AAAAAGTCTCACTTGTGATGCTGGGTCCAGCTTATAACCACACAATGGAAACCCCTGCCTC | |
| CTTCCTCCTTGTGGGTATCCCAGGACTGCAATCTTCACATCTTTGGCTATCTCACTGA | |
| GTGCCATGTACATCATAGCCCTGTTAGGAAACACCATCATCGTGACTGCAATCTGGATGGA | |
| TTCCACTCGGCATGAGCCCATGTATTGCTTTCTGTGTGTTCTGGCTGCTGTGGACATTGTTA | |
| TGGCCTCCTCGGTGGTACCCAAGATGGTGAGCATCTTCTGCTCAGGAGACAGCTCAATCAG | |
| CTTTAGTGCTTGTTTCACTCAGATGTTTTTTGTCCACTTAGCCACAGCTGTGGAGACGGGG | |
| CTGCTGCTGACCATGGCTTTTGACCGCTATGTAGCCATCTGCAAGCCTCTACACTACAAGA | |
| GAATTCTCACGCCTCAAGTGATGCTGGGAATGAGTATGGCCATCACCATCAGAGCTATCAT | |
| AGCCATAACTCCACTGAGTTGGATGGTGAGTCATCTACCTTTCTGTGGCTCCAATGTGGTT | |
| GTCCACTCCTACTGTGAGCACATAGCTTTGGCCAGGTTAGCATGTGCTGACCCCGTGCCCA | |
| GCAGTCTCTACAGTCTGATTGGTTCCTCTCTTATGGTGGGCTCTGATGTGGCCTTCATTGCT | |
| GCCTCCTATATCTTAATTCTCAAGGCAGTATTTGGTCTCTCCTCAAAGACTGCTCAGTTGAA | |
| AGCATTAAGCACATGTGGCTCCCATGTGGGGGTTATGGCTTTGTACTATCTACCTGGGATG | |
| GCATCCATCTATGCGGCCTGGTTGGGGCAGGATGTAGTGCCCTTGCACACCCAAGTCCTGC | |
| TAGCTGACCTGTACGTGATCATCCCAGCCACCTTAAATCCCATCATCTATGGCATGAGGAC | |
| CAAACAACTGCGGGAGAGAATATGGAGTTATCTGATGCATGTCCTCTTTGACCATTCCAAC | |
| CTGGGTTCATGA | |
| AOLFR114 sequences: | |
| MERINHTSSVSEFILLGLSSRPEDQKTLFVLFLIVYLVTITGNLLIILAIRFNPHLQTPMTTFLSFLS | (SEQ ID NO:209) |
| LTDICFTTSVVPKMLMNFLSEKKTISYAGCLTQMTTLYALGNSDSCLLAVMAFDRYVAVCDPF | |
| HYVTTMSHHHCVLLVAFSCSFPHLHSLLHTLLLNRLTFCDSNVIHHFLCDLSPVLKLSCSSIFVN | |
| EIVQMTEAPIVLVTRFLCIAFSYIRILTTVLKIPSTSGKRKAFSTCGFYLTVVTLFYGSIFCVYLQP | |
| PSTYAVKDHVATIVYTVLSSMLNPFIYSLRNKDLKQGLRKLMSKRS | |
| ATGGAAAGAATCAACCACACCAGCAGTGTCTCCGAGTTTATCCTCCTGGGACTCTCCTCCC | (SEQ ID NO:210) |
| GGCCTGAGGACCAAAAGACACTCTTTGTTCTCTTCCTCATCGTGTACCTGGTCACCATAAC | |
| AGGGAACCTGCTCATCATCCTGGCCATTCGCTTCAACCCCCATCTTCAGACCCCTATGTATT | |
| TCTTCTTGAGTTTTCTGTCTCTCACTGATATTTGCTTTACAACAAGCGTTGTCCCCAAGATG | |
| CTGATGAACTTCCTGTCAGAAAAGAAGACCATCTCCTATGCTGGGTGTCTGACACAGATGT | |
| ATTTTCTCTATGCCTTGGGCAACAGTGACAGCTGCCTTCTGGCAGTCATGGCCTTTGACCG | |
| CTATGTGGCCGTCTGTGACCCTTTCCACTATGTCACCACCATGAGCCACCACCACTGTGTCC | |
| TGCTGGTGGCCTTCTCCTGCTCATTTCCTCACCTCCACTCACTCCTGCACACACTTCTGCTG | |
| AATCGTCTCACCTTCTGTGACTCCAATGTTATCCACCACTTTCTCTGTGACCTCAGCCCTGT | |
| GCTGAAATTGTCCTGCTCTTCCATATTTGTCAATGAAATTGTGCAGATGACAGAAGCACCT | |
| ATTGTTTTGGTGACTCGTTTTCTCTGCATTGCTTTCTCTTATATACGAATCCTCACTACAGT | |
| TCTCAAGATTCCCTCTACTTCTGGGAAACGCAAAGCCTTCTCCACCTGTGGTTTTTACCTCA | |
| CCGTGGTGACGCTCTTTTATGGAAGCATCTTCTGTGTCTATTTACAGCCCCCATGCACCTAC | |
| GCTGTCAAGGACCACGTGGCAACAATTGTTTACACAGTTTTGTCATCCATGCTCAATCCTT | |
| TTATCTACAGCCTGAGAAACAAAGACCTGAAACAGGGCCTGAGGAAGCTTATGAGCAAGA | |
| GATCCTAG | |
| AOLFR115 sequences: | |
| MEGFYLRRSHELQGMGKPGRVNQTTVSDFLLLGLSEWPEEQPLLFGIFLGMYLVTMVGNLLII | (SEQ ID NO:211) |
| LAISSDPHLIITPMTTFLANLSLTDACFTSASIPKMLANIHTQSQIISYSGCLAQLTTLLMFGGLD | |
| NCLLAVMAYDRYVAICQPLHYSTSMSPQLCALMLGVCWVLTNCPALMHTLLLTRVAFCAQK | |
| AIPHFYCDPSALLKLACSDTHVNELMIITMGLLFLTVPLLLIVFSYVRIFWAVFVISSPGGRWKA | |
| FSTCGSHLTVVLLFYGSLMGVYLLPPSTYSTERESRAAVLYMVIIPTLNPFIYSLRNRDMKEALG | |
| KLFVSGKTFFL | |
| ATGGAAGGTTTTTATCTGCGCAGATCACACGAACTACAAGGGATGGGAAAACCAGGCAGA | (SEQ ID NO:212) |
| GTGAACCAAACCACTGTTTCAGACTTCCTCCTTCTAGGACTCTCTGAGTGGCCAGAGGAGC | |
| AGCCTCTTCTGTTTGGCATCTTCCTTGGCATGTACCTGGTCACCATGGTGGGGAACCTGCTC | |
| ATTATCCTGGCCATCAGCTCTGACCCACACCTCCATACTCCCATGTACTTCTCTTTGGCCAA | |
| CCTGTCATTAACTGATGCCTGTTTCACTTCTGCCTCCATCCCCAAAATGCTGGCCAACATTC | |
| ATACCCAGAGTCAGATCATCTCGTATTCTGGGTGTCTTGCACAGCTATATTTCCTCCTTATG | |
| TTTGGTGGCCTTGACAACTGCCTGCTGGCTGTGATGGCATATGACCGCTATGTGGCCATCT | |
| GCCAACCACTCCATTACAGCACATCTATGAGTCCCCAGCTCTGTGCACTAATGCTGGGTGT | |
| GTGCTGGGTGCTAACCAACTGTCCTGCCCTGATGCACACACTGTTGCTGACCCGCGTGGCT | |
| TTCTGTGCCCAGAAAGCCATCCCTCATTTCTATTGTGATCCTAGTGCTCTCCTGAAGCTTGC | |
| CTGCTCAGATACCCATGTAAACGAGCTGATGATCATCACCATGGGCTTGCTGTTCCTCACT | |
| GTTCCCCTCCTGCTGATCGTCTTCTCCTATGTCCGCATTTTCTGGGCTGTGTTTGTCATCTC | |
| ATCTCCTGGAGGGAGATGGAAGGCCTTCTCTACCTGTGGTTCTCATCTCACGGTGGTTCTG | |
| CTCTTCTATGGGTCTCTTATGGGTGTGTATTTACTTCCTCCATCAACTTACTCTACAGAGAG | |
| GGAAAGTAGGGCTGCTGTTCTCTATATGGTGATTATTCCCACGCTAAACCCATTCATTTAT | |
| AGCTTGAGGAACAGAGACATGAAGGAGGCTTTGGGTAAACTTTTTGTCAGTGGAAAAACA | |
| TTCTTTTTATGA | |
| AOLFR116 sequences: | |
| MDEANHSVVSEFVFLGLSDSRKIQLLLFLFFSVFYVSSLMGNLLIVLTVTSDPRLQSPMTTLLAN | (SEQ ID NO:213) |
| LSIINLVFCSSTAPKMIYDLFRKHKTISFGGCVVQIFFIHAVGGTEMVLLIAMAFDRYVAICKPLH | |
| YLTIMNPQRCILFLVISWIIGIIHSVIQLAFVVDLLFCGPNELDSFFCDLPRFIKLACIETYTLGFMV | |
| TANSGFISLASFLILIISYIFILVTVQKKSSGGIFKAFSMLSAHVIVVVLVFGPLIFFYIFPFPTSHLD | |
| KFLAIFDAVITPVLNPVIYTFRNKEMMVAMRRRCSQFVNYSKIF | |
| ATGGATGAAGCCAATCACTCTGTGGTCTCTGAGTTTGTGTTCGTGGGACTCTCTGACTCGC | (SEQ ID NO:214) |
| GGAAGATCCAGCTCCTCCTCTTCCTCTTTTTCTCAGTGTTCTATGTATCAAGCCTGATGGGA | |
| AATCTCCTCATTGTGCTAACTGTGACCTCTGACCCTCGTTTACAGTCCCCCATGTACTTCCT | |
| GCTGGCCAACCTTTCCATCATCAATTTGGTATTTTGTTCCTCCACAGCTCCCAAGATGATTT | |
| ATGACCTTTTCAGGAAGCACAAGACCATCTCTTTTGGGGGCTGTGTAGTTCAGATCTTCTT | |
| TATCCATGCAGTTGGGGGAACTGAGATGGTGCTGCTCATAGCCATGGCTTTTGACCGATAT | |
| GTGGGCATATGTAAGCCTCTCCACTACCTGACCATCATGAACCCACAAAGGTGCATTTTGT | |
| TTTTAGTCATTTCCTGGATTATAGGTATTATTCACTCAGTGATTCAGTTGGCTTTTGTTGTA | |
| GACCTGCTGTTCTGTGGCCCTAATGAATTAGATAGTTTCTTTTGTGATCTTCCTCGATTTAT | |
| CAAACTGGCTTGCATAGAGACCTACACATTGGGATTCATGGTTACTGCCAATAGTGGATTT | |
| ATTTCTCTGGCTTCTTTTTTAATTCTCATAATCTCTTACATCTTTATTTTGGTGACTGTTCAG | |
| AAAAAATCTTCAGGTGGTATATTCAAGGCTTTCTCTATGCTGTCAGCTCATGTCATTGTGG | |
| TGGTTTTGGTCTTTGGGCCATTAATCTTTTTCTATATTTTTCCATTTCCCACATCACATCTTG | |
| ATAAATTCCTTGCCATCTTTGATGCAGTTATCACTCCCGTTTTGAATCCAGTCATCTATACT | |
| TTTAGAAATAAAGAGATGATGGTGGCAATGAGAAGACGATGCTCTCAGTTTGTGAATTAC | |
| AGTAAAATCTTTTAA | |
| AOLFR117 sequences: | |
| MNNTIVFVIKIQIEKSDLKYRAISLQEISKISLLFWVLLLVISRLLLAMTLGNSTEVTEFYLLGFGA | (SEQ ID NO:215) |
| QHEFWCILFIVFLLIYVTSIMGNSGIILLINTDSRFQTLTTTFLQHLAFVDICYTSAITPKMLQSFT | |
| EEKNLILFQGCVIQFLVYATFATSDCYLLAMMAVDPYVAICKIPLHYTVIMSRTVCIRLVAGSYI | |
| MGSINASVQTGFTCSLSFCKSNSINHFFCDVPPILALSCSNVDINIMLLVVFVGSNLIFTGLVVIFS | |
| YIYIMATILKMSSSAGRKKSFSTCASHLTAVTIFYGTLSYMYLQSHSNNSQENMKVAFIFYGTVI | |
| PMLNPLIYSLRNKEVKEALKVIGKKLF | |
| ATGAATAACACTATTGTATTTGTCATAAAAATACAAATAGAAAAAAGTGACTTGAAATATA | (SEQ ID NO:216) |
| GAGCCATTTCATTGCAAGAAATCTCAAAGATTTCCCTTCTTTTCTGGGTCCTTCTCTTGGTC | |
| ATTTCTAGACTTTTACTAGCCATGACACTAGGAAACAGCACTGAAGTCACTGAATTCTATC | |
| TTCTGGGATTTGGTGCCCAGCATGAGTTTTGGTGTATCCTCTTCATTGTATTCCTTCTCATC | |
| TATGTGACCTCCATAATGGGTAATAGTGGAATAATCTTACTCATCAACACAGATTCCAGAT | |
| TTCAAACACTCACGTACTTTTTTCTACAACATTTGGCTTTTGTTGATATCTGTTACACTTCT | |
| GCTATCACTCCCAAGATGCTCCAAAGCTTCACAGAAGAAAAGAATTTGATATTATTTCAGG | |
| GCTGTGTGATACAATTCTTAGTTTATGCAACATTTGCAACCAGTGACTGTTATCTCCTGGCT | |
| ATGATGGCAGTGGATCCTTATGTTGCCATCTGTAAGCCCCTTCACTATACTGTAATCATGT | |
| CCCGAACAGTCTGCATCCGTTTGGTAGCTGGTTCATACATCATGGGCTCAATAAATGCCTC | |
| TGTACAAACAGGTTTTACATGTTCACTGTCCTTCTGCAAGTCCAATAGCATCAATCACTTTT | |
| TCTGTGATGTTCCCCCTATTCTTGCTCTTTCATGCTCCAATGTTGACATCAACATCATGCTA | |
| CTTGTTGTCTTTGTGGGATCTAACTTGATATTCACTGGGTTGGTCGTCATCTTTTCCTACAT | |
| CTACATCATGGCCACCATCCTGAAAATGTCTTCTAGTGCAGGAAGGAAAAAATCCTTCTCA | |
| ACATGTGCTTCCCACCTGACCGCAGTCACCATTTTCTATGGGACACTCTCTTACATGTATTT | |
| GCAGTCTCATTCTAATAATTCCCAGGAAAATATGAAAGTGGCCTTTATATTTTATGGCACA | |
| GTTATTCCCATGTTAAATCCTTTAATCTATAGCTTGAGAAATAAGGAAGTAAAAGAAGCTT | |
| TAAAAGTGATAGGGAAAAAGTTATTTTAA | |
| AOLFR118 sequences: | |
| MNHMSASLKISNSSKFQVSEFILLGFPGIHSWQHWLSLPLALLYLSALAANTLILIIIWQNPSLQQ | (SEQ ID NO:217) |
| PMYIFLGILCMVDMGLATTIIPKILAIFWFDAKVISLPECFAQIYAIHFFVGMESGILLCMAFDRY | |
| VAICHPLRYPSIVTSSLILKATLFMVLRNGLFVTPVPVLAAQRDYCSKNEIEHCLCSNLGVTSLA | |
| CDDRRPNSICQLVLAWLGMGSDLSLIILSYILILYSVLRLNSAEAAAKALSTCSSHLTLILFFYTIV | |
| VVISVTHLTEMKATLIPVLLNVLHNIIPPSLNPTVYALQTKELRAAFQKVLFALTKEIRS | |
| ATGAATCATATGTCTGCATCTCTCAAAATCTCCAATAGCTCCAAATTCCAGGTCTCTGAGTT | (SEQ ID NO:218) |
| CATCCTGCTGGGATTCCCGGGCATTCACAGCTGGCAACACTGGCTATCTCTGCCCCTGGCA | |
| CTACTGTATCTCTCAGCACTTGCTGCAAACACCCTCATCCTCATCATCATCTGGCAGAACCC | |
| TTCTTTACAGCAGCCCATGTATATTTTCCTTGGCATCCTCTGTATGGTAGACATGGGTCTGG | |
| CCACTACTATCATCCCTAAGATCCTGGCCATCTTCTGGTTTGATGCCAAGGTTATTAGCCTC | |
| CCTGAGTGCTTTGCTCAGATTTATGCCATTCACTTCTTTGTGGGCATGGAGTCTGGTATCCT | |
| ACTCTGCATGGCTTTTGATAGATATGTGGCTATTTGTCACCCTCTTCGCTATCCATCAATTG | |
| TCACCAGTTCCTTAATCTTAAAAGCTACCCTGTTCATGGTGCTGAGAAATGGCTTATTTGTC | |
| ACTCCAGTGCCTGTGCTTGCAGCACAGCGTGATTATTGCTCCAAGAATGAAATTGAACACT | |
| GCCTGTGCTCTAACCTTGGGGTCACAAGCCTGGCTTGTGATGACAGGAGGCCAAACAGCAT | |
| TTGCCAGTTGGTTCTGGCATGGCTTGGAATGGGGAGTGATCTAAGTCTTATTATACTGTCA | |
| TATATTTTGATTCTGTACTCTGTACTTAGACTGAACTCAGCTGAAGCTGCAGCCAAGGCCC | |
| TGAGCACTTGTAGTTCACATCTCACCCTCATCCTTTTCTTTTACACTATTGTTGTAGTGATT | |
| TCAGTGACTCATCTGACAGAGATGAAGGCTACTTTGATTCCAGTTCTACTTAATGTGTTGC | |
| ACAACATCATCCCCCCTTCCCTCAACCCTACAGTTTATGCACTTCAGACCAAAGAACTTAG | |
| GGCAGCCTTCCAAAAGGTGCTGTTTGCCCTTACAAAAGAAATAAGATCTTAG | |
| AOLFR119 sequences: | |
| MPLFNSLCWFPTIHVTPPSFILNGIPGLERVHVWISLPLCTMYIIFLVGNLGLVYLIYYEESLHHP | (SEQ ID NO:219) |
| MTTFFGHALSLIDLLTCTTTLPNALCIFWFSLKEINFNACLAQMFFVHGFTGVESGVLMLMALD | |
| RYIAICYPLRYATTLTNPIIAKAELATFLRGVLLMIPFPFLVKRLPFCQSNIISHTYCDHMSVVKL | |
| SCASIKVNVIYGLMVALLIGVFDICCISLSYTLILKAISLSSSDARQKAFSTCTAHISAIIITYVPA | |
| FFTFFAHRFGGHTIPPSLHIIVANLYLLLPPTLNPIVYGVKTKQIRKSVIKFFQGDKGAG | |
| ATGCCTCTATTTAATTCATTATGCTGGTTTCCAACAATTCATGTGACTCCTCCATCTTTTAT | (SEQ ID NO:220) |
| TCTTAATGGAATACCTGGTCTGGAAAGAGTACATGTATGGATCTCCCTCCCACTCTGCACA | |
| ATGTACATCATCTTCCTTGTGGGGAATCTTGGTCTTGTGTACCTCATTTATTATGAGGAGTC | |
| CTTACATCATCCGATGTATTTTTTTTTTGGCCATGCTCTCTCCCTCATTGACCTCCTTACCTG | |
| CACCACCACTCTACCCAATGCACTCTGCATCTTCTGGTTCAGTCTCAAAGAAATTAACTTCA | |
| ATGCTTGCTTGGCCCAGATGTTCTTTGTTCATGGGTTCACAGGTGTGGAGTCTGGGGTGCT | |
| CATGCTCATGGCTCTAGACCGCTATATAGCCATTTGCTACCCTTTGCGTTATGCTACCACAC | |
| TCACCAACCCTATCATTGCCAAGGCTGAGCTTGCCACCTTCCTGAGGGGTGTATTGCTGAT | |
| GATTCCTTTCCCATTCTTGGTTAAGCCTTTGCCTTTCTGCCAAAGCAATATTATCTCCCATA | |
| CGTACTGCGACCACATGTCTGTAGTAAAGCTATCTTGTGCCAGCATCAAGGTCAATGTAAT | |
| CTATGGTCTAATGGTTGCTCTCCTGATTGGAGTGTTTGACATTTGTTGTATATCTTTGTCTT | |
| ACACTTTGATCCTCAAGGCAGCGATCAGCCTCTCTTCATCAGATGCTCGGCAGAAGGCTTT | |
| CAGCACCTGCACTGCCCATATATCTGCCATCATCATCACCTATGTTCCAGCATTCTTCACTT | |
| TCTTTGCCCACCGTTTTGGGGGACACACAATTCCCCCTTCTCTTCACATCATTGTGGCTAAT | |
| CTTTATCTTCTTCTTCCCCCAACTCTAAACCCTATTGTTTATGGAGTAAAGACAAAACAGAT | |
| ACGCAAGAGTGTCATAAAGTTCTTCCAGGGTGATAAGGGTGCAGGTTGA | |
| AOLFR120 sequences: | |
| MQPYTKNWTQVTEFVMMGFAGIHEAHLLFFILFLTMYLFTLVENLAIILVVGLDHRLRRPMTT | (SEQ ID NO:221) |
| FLTHLSCLEIWYTSVTVPKMLAGFIGVDGGKNISYAGCLSQLFIFTFLGATECFLLAAMAYDRY | |
| VAICMPLHYGAFVSWGTCIRLAAACWLVGFLTPILPIYLLSQLTFCGPNVIDHFSCDASPLLALS | |
| CSDVTWKETVDFLVSLAVLLASSMVIAVSYGNIVWTLLHIRSAAERWKAFSTCAAHLTVVSLF | |
| YGTLFFMYVQTKVTSSINFNKVVSVFYSVVTPMLNPLIYSLRNKEVKGALGRVFSLNFWKGQ | |
| ATGCAACCATATACCAAAAACTGGACCCAGGTAACTGAATTTGTCATGATGGGCTTTGCTG | (SEQ ID NO:222) |
| GCATCCATGAAGCACACCTCCTCTTCTTCATACTCTTCCTCACCATGTACCTGTTCACCTTG | |
| GTGGAGAATTTGGCCATCATTTTAGTGGTGGGTTTGGACCACCGACTACGGAGACCCATGT | |
| ATTTCTTCCTGACACACTTGTCCTGCCTTGAAATCTGGTACACTTCTGTTACAGTGCCCAAG | |
| ATGCTGGCTGGTTTTATTGGGGTGGATGGTGGCAAGAATATCTCTTATGCTGGTTGCCTAT | |
| CCCAGCTCTTCATCTTCACCTTTCTTGGGGCAACTGAGTGTTTCCTACTGGCTGCCATGGCC | |
| TATGATCGTTATGTGGCCATTTGTATGCCTCTCCACTATGGGGCTTTTGTGTCCTGGGGCAC | |
| CTGCATCCGTCTGGCAGCTGCCTGTTGGCTGGTAGGTTTCCTCACACCCATCTTGCCAATCT | |
| ACCTCTTGTCTCAGCTAACATTTTGTGGCCCAAATGTCATTGACCATTTCTCCTGTGATGCC | |
| TCACCCTTGCTAGCCTTGTCGTGCTCAGATGTCACTTGGAAGGAGACTGTGGATTTCCTGG | |
| TGTCTCTGGCTGTGCTACTGGCCTCCTCTATGGTCATTGCTGTGTCCTATGGCAACATCGTC | |
| TGGACACTGCTGCACATCCGCTCAGCTGCTGAGCGCTGGAAGGCCTTCTCTACCTGTGCAG | |
| CTCACCTGACTGTGGTGAGCCTCTTCTATGGCACTCTTTTCTTTATGTATGTCCAGACCAAG | |
| GTGACCTCCTCCATCAACTTCAACAAGGTGGTATCTGTCTTCTACTCTGTTGTCACGCCCAT | |
| GCTCAATCCTCTCATCTACAGTCTTAGGAACAAGGAAGTGAAGGGAGCTCTGGGTCGAGTC | |
| TTTTCTCTCAACTTTTGGAAGGGACAGTGA | |
| AOLFR121 sequences: | |
| MKRKNFTEVSEFIFLGFSSFGKHQITLFVVFLTVYILTLVANIIIVTIICIDHHLHTPMTTFLSMLA | (SEQ ID NO:223) |
| SSETVYTLVIVPRMLLSLIFHNQPISLAGCATQMFFFVILATNNCFLLTAMAGYDRYVAICRPLRY | |
| TVIMSKGLCAQLVCGSFGIGLTMAVLHVTAMFNLPFCGTVVDHFFCDIYPVMKISCIDTTINEII | |
| NYGVSSFVIFVPIGLIFISYVLVISSILQIASAEGRKKTFATCVSHLTVVIVHCGGASIAYLKPKSES | |
| SIEKDLVLSVTYTIITPLLNPVVYSLRNKEVKDALCRVVGRNIS | |
| ATGAAGAGAAAGAACTTCACAGAAGTGTCAGAATTCATTTTCTTGGGATTTTCTAGCTTTG | (SEQ ID NO:224) |
| GAAAGCATCAGATAACCCTCTTTGTGGTTTTCCTAACTGTCTACATTTTAACTCTGGTTGCT | |
| AACATCATCATTGTGACTATCATCTGCATTGACCATCATCTCCACACTCCCATGTATTTCTT | |
| CCTAAGCATGCTGGCTAGTTCAGAGACGGTGTACACACTGGTCATTGTGCCACGAATGCTT | |
| TTGAGCCTCATTTTTCATAACCAACCTATCTCCTTGGCAGGCTGTGCTACACAAATGTTCTT | |
| TTTTGTTATCTTGGCCACTAATAATTGCTTCCTGCTTACTGCAATGGGGTATGACCGCTATG | |
| TGGCCATCTGCAGACCCCTGAGATACACTGTCATCATGAGCAAGGGACTATGTGCCCAGCT | |
| GGTGTGTGGGTCCTTTGGCATTGGTCTGACTATGGCAGTTCTCCATGTGACAGCCATGTTC | |
| AATTTGCCGTTCTGTGGCACAGTGGTAGACCACTTCTTTTGTGACATTTACCCAGTCATGA | |
| AACTTTCTTGCATTGATACCACTATCAATGAGATAATAAATTATGGTGTAAGTTCATTTGT | |
| GATTTTTGTGCCCATAGGCCTGATATTTATCTCCTATGTCCTTGTCATCTCTTCCATCCTTC | |
| AAATTGCCTCAGCTGAGGGCCGGAAGAAGACCTTTGCCACCTGTGTCTCCCACCTCACTGT | |
| GGTTATTGTCCACTGTGGCTGTGCCTCCATTGCCTACCTCAAGCCGAAGTCAGAAAGTTCA | |
| ATAGAAAAAGACCTTGTTCTCTCAGTGACGTACACCATCATCACTCCCTTGCTGAACCCTG | |
| TTGTTTACAGTCTGAGAAACAAGGAGGTAAAGGATGCCCTATGCAGAGTTGTGGGCAGAA | |
| ATATTTCTTAA | |
| AOLFR122 sequences: | |
| MEWENQTILVEFFLKGHSVHPRLELLFFVLIFIMYVVILLGNGTLILISILDPHLHTPMTTFLGNL | (SEQ ID NO:225) |
| SFLDICYTTTSIPSTLVSFLSERKTISFSGCAVQMFLGLAMGTTECVLLGMMAFDRYVAICNPLR | |
| YPIIMSKNAYVPMAVGSWFAGIVNSAVQTTFVVQLPFCRKNVINHFSCEILAVMKLACADISGN | |
| EFLMLVATILFTLMPLLLIVISYSLIISSILKIHSSEGRSKAFSTCSAHLTVVIIFYGTILFMYMRPKS | |
| KETLNSDDLDATDKIISMFYGVMTPMMNPLIYSLRNKDVKEAVKHLPNRRFFSK | |
| ATGGAATGGGAAAACCAAACCATTCTGGTGGAATTTTTTCTGAAGGGACATTCTGTTCACC | (SEQ ID NO:226) |
| CAAGGCTTGAGTTACTCTTTTTTGTGCTAATCTTCATAATGTATGTGGTCATCCTTCTGGGG | |
| AATGGTACTCTCATTTTAATCAGCATCTTGGACCCTCACCTTCACACCCCTATGTACTTCTT | |
| TCTGGGGAACCTCTCCTTCTTGGACATCTGCTACACCACCACCTCTATTCCCTCCACACTAG | |
| TGAGCTTCCTTTCAGAAAGAAAGACCATTTCCTTTTCTGGCTGTGCAGTGCAGATGTTCCTT | |
| GGCTTGGCCATGGGGACAACAGAGTGTGTGCTTCTGGGCATGATGGCCTTTGACCGCTATG | |
| TGGCTATCTGCAACCCTCTGAGATATCCCATCATCATGAGCAAGAATGCCTATGTACCCAT | |
| GGCTGTTGGGTCCTGGTTTGCAGGGATTGTCAACTCTGCAGTACAAACTACATTTGTAGTA | |
| CAATTGCCTTTCTGCAGGAAGAATGTCATCAATCATTTCTCATGTGAAATTCTAGCTGTCAT | |
| GAAGTTGGCCTGTGCTGACATCTCAGGCAATGAGTTCCTCATGCTTGTGGCCACAATATTG | |
| TTCACATTGATGCCACTGCTCTTGATAGTTATCTCTTACTCATTAATCATTTCCAGCATCCT | |
| CAAGATTCACTCCTCTGAGGGGAGAAGCAAAGCTTTCTCTACCTGCTCAGCCCATCTGACT | |
| GTGGTCATAATATTCTATGGGACCATCCTCTTCATGTATATGAAGCCCAAGTCTAAAGAGA | |
| CACTTAATTCAGATGACTTGGATGCTACCGACAAAATTATATCCATGTTCTATGGGGTGAT | |
| GACTCCCATGATGAATCCTTTAATCTACAGTCTTAGAAACAAGGATGTGAAAGAGGCAGT | |
| AAAACACCTACCGAACAGAAGGTTCTTTAGCAAGTGA | |
| AOLFR123 sequences: | |
| MYRFTDFDVSNISIYLNHVLFYTTQQAGDLEHMETRNYSAMTEFFLVGLSQYPELQLFLFLLCL | (SEQ ID NO:227) |
| IMYMIILLGNSLLIIITILDSRLHTPMTTFLGNLSFLDICYTSSSIPPMLIIFMSERKSISFIGCALQM | |
| VVSLGLGSTECVLLAVMAYDHYVAICNPLRYSIIMNGVLYVQMAAWSWIIGCLTSLLQTVLT | |
| MMLPFCGNNVIDHITCEILALLKLVCSDITINVLIMTVTNIVSLVILLLLIFISYVFILSSILRINCAE | |
| GRKKAFSTCSAHSIVVILFYGSALFMYMKPKSKNTNTSDEIIGLSYGVVSPMLNPIIYSLRNKEV | |
| KEAVKKVLSRHLHLLKM | |
| ATGTACAGATTTACAGATTTTGATGTATCAAACATTTCAATTTACCTGAATCATGTCCTTTT | (SEQ ID NO:228) |
| CTATACTACCCAGCAGGCAGGTGACCTAGAACACATGGAGACAAGAAATTACTCTGCCAT | |
| GACTGAATTCTTTCTGGTGGGGCTTTCCCAATATCCAGAGCTCGAGCTTTTTCTGTTCCTGC | |
| TCTGCCTCATCATGTACATGATAATCCTCCTGGGAAATAGCCTCCTCATTATCATCACCATC | |
| TTGGATTCTCGCCTCCATACTGCCATGTATTTCTTTCTTGGAAACCTCTCATTCTTGGACAT | |
| CTGTTACACATCCTCATCCATTCCTCCAATGCTTATTATATTTATGTCTGAGAGAAAATCCA | |
| TCTCCTTCATTGGCTGTGCTCTGCAGATGGTTGTGTCCCTTGGCTTGGGCTCCACTGAGTGT | |
| GTCCTCCTGGCTGTGATGGCCTATGACCACTATGTGGCCATCTGCAACCCACTGAGGTACT | |
| CCATCATCATGAACGGAGTGCTGTATGTGCAAATGGCTGCATGGTCCTGGATCATAGGCTG | |
| TCTGACCTCCCTATTGCAAACAGTTCTGACAATGATGTTGCCTTTCTGTGGGAATAATGTC | |
| ATTGATCATATTACCTGTGAAATTTTGGCCCTTCTAAAACTTGTTTGTTCAGATATCACCAT | |
| CAATGTGCTTATCATGACAGTGACAAATATTGTTTCACTGGTGATTCTTCTACTGTTAATTT | |
| TCATCTCCTATGTGTTTATTCTCTCTTCCATCCTGAGAATTAATTGTGCTGAGGGAAGAAAG | |
| AAAGCCTTCTCTACCTGTTCAGCGCACTCGATTGTGGTCATCTTATTCTACGGTTCAGCCCT | |
| TTTTATGTACATGAAACCCAAGTCAAAGAACACTAATACATCTGATGAGATTATTGGGCTG | |
| TCTTATGGAGTGGTAAGCCCAATGTTAAATCCCATCATCTATAGCCTCAGGAATAAAGAGG | |
| TCAAAGAGGCTGTAAAGAAAGTCCTGAGCAGACATCTGCATTTATTGAAAATGTGA | |
| AOLFR124 sequences: | |
| MNHSVVTEFIILGLTKKPELQGIIFLFFLIVYLVAFLGNMLIIIAKIYNNTLHTPMYVFLLTLAVV | (SEQ ID NO:229) |
| DIICTTSIIPKMLGTMLTSENTISYAGCMSQLFLFTWSLGAEMVLFTTMAYDRYVAICFPLHYST | |
| VMNHHMCVALLSMVMAIAVTNSWVHTALIMRLTFCGPNTIDHFFCEIPPLLALSCSPVRINEV | |
| MVYVADITLAIGDFILTCISYGFIIVAILRIRTVEGKRKAFSTCSSHLTVVTLYYSPVIYTYIRPASS | |
| YTFERDKVVAALYTLVTPTLNPMVYSFQNREMQAGIRKVEAFLKH | |
| ATGAATCACAGCGTTGTAACTGAGTTCATTATTCTGGGCCTCACCAAAAAGCCTGAACTCC | (SEQ ID NO:230) |
| AGGGAATTATCTTCCTCTTTTTTCTCATTGTCTATCTTGTGGCTTTTCTCGGCAACATGCTC | |
| ATCATCATTGCCAAAATCTATAACAACACCTTGCATACGCCCATGTATGTTTTCCTTCTGAC | |
| ACTGGCTGTTGTGGACATCATCTGCACAACAAGCATCATACCGAAGATGCTGGGGACCAT | |
| GCTAACATCAGAAAATACCATTTCATATGCAGGCTGCATGTCCCAGCTCTTCTTGTTCACA | |
| TGGTCTCTGGGAGCTGAGATGGTTCTCTTCACCACCATGGCCTATGACCGCTATGTGGCCA | |
| TTTGTTTCCCTCTTCATTACAGTACTGTTATGAACCACCATATGTGTGTAGCCTTGCTCAGC | |
| ATGGTCATGGCTATTGCAGTCACCAATTCCTGGGTGCACACAGCTCTTATCATGAGGTTGA | |
| CTTTCTGTGGGCCAAACACCATTGACCACTTCTTCTGTGAGATACCCCCATTGCTGGCTTTG | |
| TCCTGTAGCCCTGTAAGAATCAATGAGGTGATGGTGTATGTTGCTGATATTACCCTGGCCA | |
| TAGGGGACTTTATTCTTACCTGCATCTCCTATGGTTTTATCATTGTTGCTATTCTCCGTATC | |
| CGCACAGTAGAAGGCAAGAGGAAGGCCTTCTCAACATGCTCATCTCATCTCACAGTGGTG | |
| ACCCTTTACTATTCTCCTGTAATCTACACCTATATCCGCCCTGCTTCCAGCTATACATTTGA | |
| AAGAGACAAGGTGGTAGCTGCACTCTATACTCTTGTGACTCCCACATTAAACCCGATGGTG | |
| TACAGCTTCCAGAATAGGGAGATGCAGGCAGGAATTAGGAAGGTGTTTGCATTTCTGAAA | |
| CACTAG | |
| AOLFR125 sequences: | |
| MTNQTQMMEFLLVRFTENWVLLRLHALLFSLIYLTAVLMNLVIILLMILDHRLHMAMTTFLRH | (SEQ ID NO:231) |
| LSFLDLCLISATVPKSILNSVASTDSISFLGCVLQLFLVVLLAGSEIGILTAMSYDRYAAICCPLHC | |
| EAVMSRGLCVQLMALSWLNRGALGLLYTAGTFSLNFYGSDELHQFFCDVPALLKLTCSKEHAI | |
| ISVSVAIGVCYAFSCLVCIVVSYVYIFSAVLRISQRQRQSKAFSNCVPHLIVVTVFLVTGAVAYL | |
| KPGSDAPSILDLLVSVFYSVAPPTLNPVIYCLKNKDIKSALSKVLWNVRSSGVMKDD | |
| ATGACCAATCAGACACAGATGATGGAATTCTTGCTTGTGAGATTTACTGAGAATTGGGTGC | (SEQ ID NO:232) |
| TCCTGAGGCTGCATGCTTTGCTCTTCTCACTGATCTACCTCACGGCTGTGCTGATGAATTTA | |
| GTCATCATTCTCCTCATGATTCTGGACCATCGTCTCCACATGGCAATGTACTTTTTCCTCCG | |
| ACATTTGTCCTTCTTAGACCTGTGTCTCATTTCTGCCACAGTCCCCAAATCCATCCTCAACT | |
| CTGTCGCCTCCACTGACTCCATCTCCTTCCTGGGGTGTGTGTTGCAGCTCTTCTTGGTGGTA | |
| CTGCTGGCTGGATCAGAGATTGGCATCCTTACTGCCATGTCCTATGACCGCTATGCTGCCA | |
| TCTGCTGCCCCCTACACTGTGAGGCTGTCATGAGCAGAGGGCTCTGTGTCCAGTTGATGGC | |
| TCTGTCCTGGCTCAACAGAGGGGCCTTGGGACTCTTGTACACAGCTGGAACATTCTCTCTG | |
| AATTTTTATGGCTCTGATGAGCTACATCAGTTCTTCTGCGATGTCCCTGCCCTACTAAAGCT | |
| CACTTGTTCTAAAGAACATGCCATCATTAGTGTCAGTGTGGCCATTGGGGTCTGTTATGCA | |
| TTTTCATGTTTAGTTTGCATTGTAGTTTCCTATGTGTACATTTTCTCTGCTGTGTTAAGGAT | |
| ATCACAGAGACAGAGACAATCCAAAGCCTTTTCCAACTGTGTGCCTCACCTCATTGTTGTC | |
| ACTGTGTTTCTTGTAACAGGTGCTGTTGCTTATTTAAAGCCAGGGTCTGATGCACCTTCTAT | |
| TCTAGACTTGCTGGTGTCTGTGTTCTATTCTGTCGCACCTCCAACCTTGAACCCTGTTATCT | |
| ACTGTCTGAAGAACAAGGACATTAAATCCGCTCTGAGTAAAGTCCTGTGGAATGTTAGAA | |
| GCAGTGGGGTAATGAAAGATGACTAA | |
| AOLFR126 sequences: | |
| MFLYLCFIFQRTCSEEMEEENATLLTEFVLTGFLHQPDCKIPLFLAFLVIYLITIMGNLGLIVLIW | (SEQ ID NO:233) |
| KDPHLHIPMYLFLGSLAFVDASLSSTVTPKMLINFLAKSKMISLSECMVQFFSLVTTVTTECFLL | |
| ATMAYDRYVAICKALLYPVIMTNELCIQLLVLSFIGGLLHALIHEAFSFRLTFCNSNIIQHFYCDII | |
| PLLKISCTDSSINFLMVFIFAGSVQVFTIGTILISYTIILFTILEKKSIKGIRKAVSTCGAHLLSVSLY | |
| YGPLTFKYLGSASPQADDQDMMESLFYTVIVPLLNPMIYSLRNKQVIASFTKMFKSNV | |
| ATGTTCCTTTACCTTTGCTTCATTTTTCAGAGGACATGCAGTGAGGAGATGGAAGAGGAAA | (SEQ ID NO:234) |
| ATGCAACATTGCTGACAGAGTTTGTTCTCACAGGATTTTTACATCAACCTGACTGTAAAAT | |
| ACCGCTCTTCCTGGCATTCTTGGTAATATATCTCATCACCATCATGGGGAATCTTGGTCTAA | |
| TTGTTCTCATCTGGAAAGACCCTCACCTTCATATCCCAATGTACTTATTCCTTGGGAGTTTA | |
| GCCTTTGTGGATGCTTCGTTATCATCCACAGTGACTCCGAAGATGCTGATCAACTTCTTAG | |
| CTAAGAGTAAGATGATATCTCTCTCTGAATGCATGGTACAATTTTTTTCCCTTGTAACCACT | |
| GTAACCACAGAATGTTTTCTCTTGGCAACAATGGCATATGATCGCTATGTAGCCATTTGCA | |
| AAGCTTTACTTTATCCAGTCATTATGACCAATGAACTATGCATTCAGCTATTAGTCTTGTCA | |
| TTTATAGGTGGCCTTCTTCATGCTTTAATCCATGAAGCTTTTTCATTCAGATTAACCTTCTG | |
| TAATTCCAACATAATACAACACTTTTACTGTGACATTATCCCATTGTTAAAGATTTCCTGTA | |
| CTGATTCCTCTATTAACTTTCTAATGGTTTTTATTTTCGCAGGTTCTGTTCAAGTTTTTACCA | |
| TTGGAACTATTCTTATATCTTATACAATTATCCTCTTTACAATCTTAGAAAAGAAGTCTATC | |
| AAAGGGATACGAAAAGCTGTCTCCACCTGTGGGGCTCATCTCTTATCTGTATCTTTATACT | |
| ATGGCCCCCTCACCTTCAAATATCTGGGCTCTGCATCTCCGCAAGCAGATGACCAAGATAT | |
| GATGGAGTCTCTATTTTACACTGTCATAGTTCCTTTATTAAATCCCATGATCTACAGCCTGA | |
| GAAACAAGCAAGTAATAGCTTCATTCACAAAAATGTTCAAAAGCAATGTTTAG | |
| AOLFR127 sequences: | |
| MSNEDMEQDNTTLLTEFVLTGLTYQPEWKMPLFLVFLVIYLITIVWNLGLIALIWNDPQLHIPM | (SEQ ID NO:235) |
| TTFLGSLAFVDAWISSTVTPKMLVNFLAKNRMISLSECMIQFFSFAFGGTTECFLLATMAYDRY | |
| VAICKPLLYPVIMNNSLCIRLLAFSFLGGFLHALIHEVLIFRLTFCNSNIIHHFYCDIIPLFMISCTD | |
| PSINFLMVFILSGSIQVFTIVTVLNSYTFALFTILKKKSVRGVRKAFSTCGAHLLSVSLYYGPLIF | |
| MYLRPASPQADDQDMIDSVFYTIIIPLLNPIIYSLRNKQVIDSFTKMVKRNV | |
| ATGTCGAATGAGGACATGGAACAGGATAATACAACATTGCTGACAGAGTTTGTTCTCACA | (SEQ ID NO:236) |
| GGACTTACATATCAGCCAGAGTGGAAAATGCCCCTGTTCTTGGTGTTCTTGGTGATCTATC | |
| TCATCACTATTGTGTGGAACCTTGGTCTGATTGCTCTTATCTGGAATGACCCACAACTTCAC | |
| ATCCCCATGTACTTTTTTCTTGGGAGTTTAGCCTTTGTTGATGCTTGGATATCTTCCACAGT | |
| AACTCCCAAAATGTTGGTTAATTTCTTGGCCAAAAACAGGATGATATCTCTGTCTGAATGC | |
| ATGATTCAATTTTTTTCCTTTGCATTTGGTGGAACTACAGAATGTTTTCTCTTGGCAACAAT | |
| GGCATATGATCGCTATGTAGCCATATGCAAACCTTTACTATATCCAGTGATTATGAACAAT | |
| TCACTATGCATACGGCTGTTAGCCTTCTCATTTTTAGGTGGCTTCCTCCATGCCTTAATTCA | |
| TGAAGTCCTTATATTCAGATTAACCTTCTGCAATTCTAACATAATACATCATTTTTACTGTG | |
| ATATTATACCACTGTTTATGATTTCCTGTACTGACCCTTCTAATTAATTTTCTAATGGTTTTTA | |
| TTTTGTCTGGCTCAATTCAGGTATTCACCATTGTGACAGTTCTTAATTCTTACACATTTGCT | |
| CTTTTCACAATCCTAAAAAAGAAGTCTGTTAGAGGCGTAAGGAAAGCCTTTTCCACCTGTG | |
| GAGCCCATCTCTTATCTGTCTCTTTATATTATGGCCCACTTATCTTCATGTATTTGCGCCCT | |
| GCATCTCCACAAGCAGATGACCAAGATATGATAGACTCTGTCTTTTATACAATCATAATTC | |
| CTTTGCTAAATCCCATTATCTACAGTCTGAGAAATAAACAAGTAATAGATTCATTCACAAA | |
| AATGGTAAAAAGAAATGTTTAG | |
| AOLFR128 sequences: | |
| METQNLTVVTEFILLGLTQSQDAQLLVFVLVLIFYLIILPGNFLIIFTIKSDPGLTAPLTTFLGNLA | (SEQ ID NO:237) |
| LLDASYSFIVVPRMLVDFLSEKKVISYRSCITQLFFLHFLGAGEMFLLVVMAFDRYIAICRPLHY | |
| STIMNPRACYALSLVLWLGGFIHSIVQVALILHLPFCGPNQLDNFFCDVPQVIKLACTNTFVVEL | |
| LMVSNSGLLSLLCFLGLLASYAVILCRIREHSSEGKSKAISTCTTHIIIIFLMFGPAIFIYTCPFQAFP | |
| ADKVVSLFHTVIFPLMNPVIYTLRNQEVKASMRKLLSQHMFC | |
| ATGGAAACACAGAACCTCACAGTGGTGACAGAATTCATTCTTCTTGGTCTGACCCAGTCTC | (SEQ ID NO:238) |
| AAGATGCTCAACTTCTGGTCTTTGTGCTAGTCTTAATTTTCTACCTTATCATCCTCCCTGGA | |
| AATTTCCTCATCATTTTCACCATAAAGTCAGACCCTGGGCTCACAGCCCCCCTCTATTTCTT | |
| TCTGGGCAACTTGGCCTTACTGGATGCATCCTACTCCTTCATTGTGGTTCCCAGGATGTTG | |
| GTGGACTTCCTCTCTGAGAAGAAGGTAATCTCCTATAGAAGCTGCATCACTCAGCTCTTTT | |
| TCTTGCATTTTCTTGGAGCGGGAGAGATGTTCCTCCTCGTTGTGATGGCCTTTGACCGCTAC | |
| ATCGCCATCTGCCGGCCTTTACACTATTCAACCATCATGAACCCTAGAGCCTGCTATGCAT | |
| TATCGTTGGTTCTGTGGCTTGGGGGCTTTATCCATTCCATTGTACAAGTAGCCCTTATCCTG | |
| CACTTGCCTTTCTGTGGCCCAAACCAGCTCGATAACTTCTTCTGTGATGTTCCACAGGTCAT | |
| CAAGCTGGCCTGCACCAATACCTTTGTGGTGGAGCTTCTGATGGTCTCCAACAGTGGCCTG | |
| CTCAGCCTCCTGTGCTTCCTGGGCCTTCTGGCCTCCTATGCAGTCATCCTCTGTCGTATAAG | |
| GGAGCACTCCTCTGAAGGAAAGAGCAAGGCTATTTCCACATGCACCACCCATATTATCATT | |
| ATATTTCTCATGTTTGGACCTGCTATTTTCATCTACACTTGCCCCTTCCAGGCTTTCCCAGC | |
| TGACAAGGTAGTTTCTCTTTTCCATACTGTCATCTTTCCTTTGATGAACCCTGTTATTTATA | |
| CGCTTCGCAACCAGGAGGTGAAAGCTTCCATGAGGAAGTTGTTAAGTCAACATATGTTTTG | |
| CTGA | |
| AOLFR129 sequences: | |
| MALTTSLILHGMSDLFFLSTGHPRASCRMEAMKLLNQSQVSEFILLGLTSSQDVEFLLFALFSVI | (SEQ ID NO:239) |
| YVVTVLGNLLIIVTVFNTPNLNTPMTTLLGNLSFVDMTLASFATPKVILNLLKKQKVISFAGCFT | |
| QIFLLHLLGGVEMVLLVSMAFDRYVAICKPLHYMTIMNKKVCVLLVVTSWLLGLLHSGFQIPF | |
| AVNLPFCGPNVVDSIFCDLPLVTKLACIDITTVQVVIVANSGIISLSCFIILLISYSLILITIKNHSPT | |
| GQSKARSTLTAHITVVILFFGPCIFIYIWPFGNHSVDKFLAVFYTIITPILNPIIYTLRNKEMKISMK | |
| KLWRAFVNSREDT | |
| ATGGCTCTTTATTTTTCACTCATACTCCATGGTATGAGTGATCTTTTCTTTCTCTCTACAGG | (SEQ ID NO:240) |
| TCATCCAAGAGCGAGCTGTAGGATGGAGGCCATGAAACTATTAAATCAATCTCAAGTGTC | |
| AGAATTCATTTTGCTGGGACTGACCAGCTCCCAGGATGTAGAGTTTCTTCTCTTTGCCCTCT | |
| TCTCGGTTATCTATGTGGTCACAGTTTTGGGTAACCTTCTTATTATAGTCACAGTGTTTAAC | |
| ACCCCTAACCTGAATACTCCCATGTATTTTCTCCTTGGTAATCTCTCTTTTGTAGATATGAC | |
| CCTTGCTTCTTTTGCCACCCCTAAGGTGATTCTGAACTTGTTAAAAAAGCAGAAGGTAATT | |
| TCTTTTGCTGGGTGCTTCACTCAGATATTTCTCCTTCACTTACTGGGTGGGGTTGAAATGGT | |
| ACTGTTGGTCTCCATGGCTTTTGACAGATATGTGGCCATTTGTAAGCCCCTACACTACATG | |
| ACCATCATGAACAAGAAGGTATGTGTTTTGCTTGTAGTGACCTCATGGCTCTTGGGTCTCC | |
| TTCACTCAGGGTTTCAGATACCATTTGCTGTGAACTTGCCCTTTTGTGGTCCCAATGTGGTA | |
| GACAGCATTTTTTGTGACCTCCCTTTGGTTACTAAGCTTGCCTGTATAGACATATATTTTGT | |
| ACAGGTAGTCATTGTTGCCAACAGTGGCATAATCTCCCTGAGCTGTTTCATTATTTTGCTTA | |
| TCTCCTACAGTCTGATCCTCATAACCATTAAGAACCACTCTCCTACTGGGCAATCTAAAGC | |
| CCGTTCCACTTTGACTGCTCACATCACAGTGGTGATTCTCTTCTTTGGCCCATGCATCTTTA | |
| TCTACATTTGGCCCTTCGGCAACCACTCTGTAGATAAGTTCCTTGCTGTGTTTTATACCATC | |
| ATCACTCCTATCTTGAATCCAATTATCTATACTCTGAGAAACAAAGAAATGAAGATATCCA | |
| TGAAAAAACTCTGGAGAGCTTTTGTGAATTCTAGAGAAGATACTTAG | |
| AOLFR131 sequences: | |
| MASTSNVTELIFTGLFQDPAVQSVCFVVFLPVYLATVVGNGLIVLTVSISKSLDSPMTTFLSCLS | (SEQ ID NO:241) |
| LVEISYSSTIAPKFIIDLLAKIKTISLEGCLTQIFFFHFFGVAEILLIVVMAYDCYVAICKPLHYMNI | |
| ISRQLCHLLVAGSWLGGFCHSIIQILVIIQLPECGPNVIDHTTCDLQPLFKLACTDTFMEGVIVLA | |
| NSGLFSVFSFLILVSSYIVILVNLRNHSAEGRHKALSTCASHITVVILFFGPAIFLYMRPSSTFTED | |
| KLVAVFYTVITPMLNPIIYTLRNAEVKIAIRRLWSKKENPGRE | |
| ATGGCCAGTACAAGTAATGTGACTGAGTTGATTTTCACTGGCCTTTTCCAGGATCCAGCTG | (SEQ ID NO:242) |
| TGCAGAGTGTATGCTTTGTGGTGTTTCTCCCCGTGTACCTTGCCACGGTGGTGGGCAATGG | |
| CCTCATCGTTCTGACGGTCAGTATCAGCAAGAGTCTGGATTCTCCCATGTACTTCTTCCTTA | |
| GCTGCCTGTCCTTGGTGGAGATCAGTTATTCCTCCACTATCGCCCCTAAATTCATCATAGAC | |
| TTACTTGCCAAGATTAAAACCATCTCTCTGGAAGGCTGTCTGACTCAGATATTCTTCTTCCA | |
| CTTCTTTGGGGTTGCTGAGATCCTTTTGATTGTGGTGATGGCCTATGATTGCTACGTGGCC | |
| ATTTGCAAGCCTCTTCATTATATGAACATTATCAGTCGTCAACTGTGTCACCTTCTGGTGGC | |
| TGGTTCCTGGCTGGGGGGCTTTTGTCACTCCATAATTCAGATTCTCGTTATCATCCAATTGC | |
| CCTTCTGTGGTCCCAATGTGATTGACCACTATTTCTGTGACCTCCAGCCTTTATTCAAGCTT | |
| GCCTGCACTGACACCTTCATGGAGGGGGTTATTGTGTTGGCCAACAGTGGATTATTCTCTG | |
| TCTTCTCCTTCCTCATCTTGGTGTCCTCTTATATTGTCATTCTGGTCAACTTGAGGAACCAT | |
| TCTGCAGAGGGGAGGCACAAAGCCCTCTCCACCTGTGCTTCTCACATCACAGTGGTCATCT | |
| TGTTTTTTGGACCTGCTATCTTCCTCTACATGCGACCTTCTTCCACTTTCACTGAAGATAAA | |
| CTTGTGGCTGTATTCTACACGGTCATCACCCCCATGCTGAACCCCATCATTTACACACTCAG | |
| GAATGCAGAGGTGAAAATCGCCATAAGAAGATTGTGGAGCAAAAAGGAGAATCCAGGGA | |
| GGGAGTGA | |
| AOLFR132 sequences: | |
| MVATNNVTEIIFVGFSQNWSEQRVISVMFLLMYTAVVLGNGLIVVTILASKVLTSPMTTFLSYL | (SEQ ID NO:243) |
| SFVEICYCSVMAPKLIFDSFIKRKVISLKGCLTQMFSLHFFGGTEAFLLMVMAYDRYVAICKPL | |
| HYMAIMNQRMCGLLVRIAWGGGLLHSVGQTFLIFQLPFCGPNIMDHTTCDVHPVLELACADT | |
| FFISLLIITNGGSISVVSFFVLMASYLIILHFLRSHNLEGQHKALSTCASHVTVVDLFFIPCSLVYIR | |
| PCVTLPADKIVAVFYTVVTPLLNPVIYSFRNAEVKNAMRRFIGGKVI | |
| ATGGTTGCTACAAACAATGTGACTGAAATAATTTTCGTGGGATTTTCCCAGAATTGGAGTG | (SEQ ID NO:244) |
| AGCAGAGGGTCATTTCTGTGATGTTTCTCCTCATGTACACAGCTGTTGTGCTGGGCAATGG | |
| CCTCATTGTGGTGACCATCCTGGCCAGCAAAGTGCTCACCTCCCCCATGTATTTCTTTCTCA | |
| GCTACTTATCCTTTGTGGAGATCTGCTACTGTTCTGTCATGGCCCCCAAGCTTATCTTTGAC | |
| TCCTTTATCAAGAGGAAAGTCATTTCTCTCAAGGGCTGCCTCACACAGATGTTTTCCCTCC | |
| ATTTCTTTGGTGGCACTGAGGCCTTTCTCCTGATGGTGATGGCCTATGACCGCTATGTGGC | |
| CATCTGCAAGCCCTTGCACTACATGGCCATCATGAACCAGCGAATGTGTGGTCTCCTCGTG | |
| AGGATAGCATGGGGCGGGGGCCTGCTGCATTCTGTTGGGCAAACCTTCCTGATTTTCCAGC | |
| TCCCGTTCTGTGGCCCCAACATCATGGACCACTACTTCTGTGATGTCCACCCAGTGCTGGA | |
| GCTGGCCTGCGCAGACACCTTCTTCATTAGCCTGCTGATCATCACCAATGGCGGCTCCATC | |
| TCCGTAGTCAGTTTCTTCGTGCTGATGGCTTCCTACCTGATCATCCTGCACTTCCTGAGAAG | |
| CCACAACTTGGAGGGGCAGCACAAGGCCCTCTCCACCTGTGCCTCTCATGTCACAGTTGTC | |
| GACCTGTTCTTCATACCTTGCTCCTTGGTCTATATTAGGCCCTGTGTCACCCTCCCTGCAGA | |
| CAAGATAGTTGCTGTATTTTATACAGTGGTCACACCTCTCTTAAACCCTGTGATTTACTCCT | |
| TCAGGAATGCTGAAGTGAAAAATGCCATGAGGAGATTTATTGGGGGAAAAGTAATTTGA | |
| AOLFR133 sequences: | |
| MTEFIFLVLSPNQEVQRVCFVIFLFLYTAIVLGNFLIVLTVMTSRSLGSPMTTFLSYLSFMEICYS | (SEQ ID NO:245) |
| SATAPKLISDLLAERKVISWWGCMAQLFFLHFFGGTEIFLLTVMAYDHYVAICKPLSYTTIMN | |
| WQVCTVLVGIAWVGGFMHSFAQILLIFHLLFCGPNVINHTTCDLVPLLKLACSDTFLIGLLIVAN | |
| GGTLSVISFGVLLASYMVILLHLRTWSSEGWCKALSTCGSHFAVVILFFGPCVFNSLRPSTTLPI | |
| DKMVAVFYTVITAILNPVIYSLRNAEMRKAMKRLWIRTLRLNEK | |
| ATGACTGAATTCATTTTTCTGGTACTTTCTCCCAACCAGGAGGTGCAGAGGGTTTGCTTTG | (SEQ ID NO:246) |
| TGATATTTCTGTTCTTGTACACAGCAATTGTGCTGGGGAATTTCCTCATTGTGCTCACTGTC | |
| ATGACCAGCAGAAGCCTTGGTTCCCCCATGTACTTCTTCCTCAGCTACCTCTCCTTCATGGA | |
| GATCTGCTACTCCTCCGCTACAGCCCCCAAACTCATCTCAGATCTGCTGGCTGAAAGGAAA | |
| GTCATATCTTGGTGGGGCTGCATGGCACAGCTTTTCTTCTTGCACTTCTTTGGTGGCACTGA | |
| GATTTTCCTGCTCACTGTGATGGCCTATGACCACTATGTGGCCATCTGCAAGCCCCTCAGC | |
| TACACCACCATCATGAACTGGCAGGTGTGTACTGTCCTTGTAGGAATAGCATGGGTGGGA | |
| GGCTTCATGCATTCCTTTGCACAAATCCTTCTCATCTTCCACCTGCTCTTCTGTGGCCCCAA | |
| TGTGATCAATCACTATTTCTGTGACCTAGTTCCCCTTCTCAAACTTGCCTGCTCTGACACCT | |
| TCCTCATTGGTCTGCTGATTGTTGCCAATGGAGGCACCCTGTCTGTGATCAGTTTTGGGGT | |
| CCTCTTAGCATCCTATATGGTCATCTTGCTCCATCTGAGAACCTGGAGCTCTGAAGGGTGG | |
| TGCAAAGCCCTCTCCACCTGTGGGTCCCATTTCGCTGTGGTTATCTTGTTCTTTGGGCCCTG | |
| CGTCTTCAACTCTCTGAGGCCTTCTACCACTCTGCCCATAGACAAGATGGTGGCTGTGTTCT | |
| ACACAGTGATAACCGCGATCCTGAACCCTGTCATCTACTCTCTGAGAAATGCTGAAATGAG | |
| GAAGGCCATGAAGAGGCTGTGGATTAGGACATTGAGACTAAATGAGAAATAG | |
| AOLFR134 sequences: | |
| MTTIILEVDNHTVTTRFILLGFPTRPAFQLLFFSIFLATYLLTLLENLLIILAIHSDGQLHKPMTTFL | (SEQ ID NO:247) |
| SHLSFLEMWYVTVISPKMLVDFLSHDKSISFNGCMTQLTTFVTFVCTEYILLAIMAFDRYVAIC | |
| NPLRYPVIMTNQLCGTLAGGCWFCGLMTAMIKMVFIAQLHYCGMPQINHTTCDISPLLNVSCE | |
| DASQAEMVDFFLALMVIAIPLCVVVASYAAILATILRIPSAQGRQKAFSTCASHLTVVILFYSMT | |
| LFTYARPKLMYAYNSNKVVSVLYTVIVPLLNPIIYCLRNHEVKAALRKTIHCRGSGPQGNGAFS | |
| S | |
| ATGACCACCATAATTCTGGAAGTAGATAATCATACAGTGACAACACGTTTCATTCTTCTGG | (SEQ ID NO:248) |
| GGTTTCCAACACGACCAGCCTTCCAGCTTCTCTTTTTCTCCATTTTCCTGGCAACCTATCTG | |
| CTGACACTGCTGGAGAATCTTCTTATCATCTTAGCTATCCACAGTGATGGGCAGCTGCATA | |
| AGCCCATGTACTTCTTCTTGAGCCACCTCTCCTTCCTGGAGATGTGGTATGTCACAGTCATC | |
| AGCCCCAAGATGCTTGTTGACTTCCTCAGTCATGACAAGAGTATTTCCTTCAATGGCTGCA | |
| TGACTCAACTTTACTTTTTTGTGACCTTTGTCTGCACTGAGTACATCCTTCTTGCTATCATG | |
| GCCTTTGACCGCTATGTAGCCATTTGTAATCCACTACGCTACCCAGTCATCATGACCAACC | |
| AGCTCTGTGGCACACTGGCTGGAGGATGCTGGTTCTGTGGACTCATGACTGCCATGATTAA | |
| GATGGTTTTTATAGCACAACTTCACTACTGTGGCATGCCTCAGATCAATCACTACTTTTGTG | |
| ATATCTCTCCACTCCTTAACGTCTCCTGTGAGGATGCCTCACAGGCTGAGATGGTGGACTT | |
| CTTCTTGGCCCTCATGGTCATTGCTATTCCTCTTTGTGTTGTGGTGGCATCCTACGCTGCTA | |
| TCCTTGCCACCATCCTCAGGATCCCTTCTGCTCAGGGCCGCCAAAAGGCATTCTCCACCTG | |
| TGCCTCCCACCTGACCGTCGTAATTCTCTTCTATTCCATGACACTTTTCACCTATGCCCGTC | |
| CCAAACTCATGTATGCCTACAATTCCAACAAAGTGGTATCTGTTCTCTACACTGTCATTGTT | |
| CCACTCCTCAACCCCATCATTTACTGTCTGAGGAACCATGAAGTAAAGGCAGCCCTCAGAA | |
| AGACCATACATTGCAGAGGAAGTGGGCCCCAGGGAAATGGGGCTTTCAGTAGTTAA | |
| AOLFR135 sequences: | |
| MIFPSHDSQAFTSVDMEVGNCTILTEFILLGFSADSQWQPILFGVFLMLYLITLSGNMTLVILIRT | (SEQ ID NO:249) |
| DSHLHTPMTTFIGNLSFLDFWYTSVYTPKILASCVSEDKRISLAGCGAQLFFSCVVAYTECYLL | |
| AAMAYDRHAAICNPLLYSGTMSTALCTGLVAGSYIGGFLNAIAHTANTFRLHFCGKNIIDHFFC | |
| DAPPLVKMSCTNTRVYEKVLLGVVGFTVLSSILAILISYVNILLAILRIHSASGRIHKAFSTCASHL | |
| ISVMLFYGSLLFMYSRPSSTYSLERDKVAALFYTVINPLLNPLIYSLRNKDIKEAFRKATQTIQPQ | |
| T | |
| ATGATTTTCCCTTCTCATGATAGTCAGGCTTTCACCTCCGTGGACATGGAAGTGGGAAATT | (SEQ ID NO:250) |
| GCACCATCCTGACTGAATTCATCTTGTTGGGTTTCTCAGCAGATTCCCAGTGGCAGCCGAT | |
| TCTATTTGGAGTGTTTCTGATGCTCTATTTGATAACCTTGTCAGGAAACATGACCTTGGTTA | |
| TCTTAATCCGAACTGATTCCCACTTGCATACACCTATGTACTTTTTCATTGGCAATCTGTGT | |
| TTTTTGGATTTCTGGTATACCTCTGTGTATACCCCCAAAATCCTGGCCAGTTGTGTCTCAGA | |
| AGATAAGCGCATTTCCTTGGCTGGATGTGGGGCTCAGCTGTTTTTTTCCTGTGTTGTAGCCT | |
| ACACTGAATGCTATCTCCTGGCAGCCATGGCATATGACCGCCATGCAGCAATTTGTAACCC | |
| ATTGCTTTATTCAGGTACCATGTCCACCGCCCTCTGTACTGGGCTTGTTGCTGGCTCCTACA | |
| TAGGAGGATTTTTGAATGCCATAGCCCATACTGCCAATACATTCCGCCTGCATTTTTGTGG | |
| TAAAAATATCATTGACCACTTTTTCTGTGATGCACCACCATTGGTAAAAATGTCCTGTACA | |
| AACACCAGGGTCTACGAAAAAGTCCTGCTTGGTGTGGTGGGCTTCACAGTACTCTCCAGCA | |
| TTCTTGCTATCCTGATTTCCTATGTCAACATCCTCCTGGCTATCCTGAGAATCCACTCAGCT | |
| TCAGGAAGACACAAGGCATTCTCCACCTGTGCTTCCCACCTCATCTCAGTCATGCTCTTCTA | |
| TGGATCATTGTTGTTTATGTATTCAAGGCCTAGTTCCACCTACTCCCTAGAGAGGGACAAA | |
| GTAGCTGCTCTGTTCTACACCGTGATCAACCCACTGCTCAACCCTCTCATCTATAGCCTGAG | |
| AAACAAAGATATCAAAGAGGCCTTCAGGAAAGCAAGACAGACTATACAACCACAAACATG | |
| A | |
| AOLFR136 sequences: | |
| MTMENYSMAAQFVLDGLTQQAELQLPLFLLFLGIYVVTVVGNLGMILLIAVSPLLHTPMYTTL | (SEQ ID NO:251) |
| SSLSFVDFCYSSVITPKMLVNFLGKKNTILYSECMVQLFFFVVFVVAEGYLLTAMAYDRYVAIC | |
| SPLLYNAIMSSWVCSLLVLAAFFLGFLSALTHTSAMMKLSFCKSHIINHTTCDVLPLLNLSCSNT | |
| HLNELLLFIIAGFNTLVPTLAVAVSYAFILYSILHIRSSEGRSKAFGTCSSHLMAVVIFFGSITFMY | |
| FKPPSSNSLDQEKVSSVFYTTVIPMLNPLIYSLRNKDVKKALRKVLVGK | |
| ATGACCATGGAAAATTATTCTATGGCAGCTCAGTTTGTCTTAGATGGTTTAACACAGCAAG | (SEQ ID NO:252) |
| CAGAGCTCCAGCTGCCCCTCTTCCTCCTGTTCCTGGGAATCTATGTGGTCACAGTAGTGGG | |
| CAACCTGGGCATGATTCTCCTGATTGCAGTCAGCCCTCTACTTCACACCCCCATGTACTATT | |
| TCCTCAGCAGCTTGTCCTTCGTCGATTTCTGCTATTCCTCTGTCATTACTCCCAAAATGCTG | |
| GTGAACTTCCTAGGAAAGAAGAATACAATCCTTTACTCTGAGTGCATGGTCCAGCTCTTTT | |
| TCTTTGTGGTCTTTGTGGTGGCTGAGGGTTACCTCCTGACTGCCATGGCATATGATCGCTA | |
| TGTTGCCATCTGTAGCCCACTGCTTTATAATGCGATCATGTCCTCATGGGTCTGCTCACTGC | |
| TAGTGCTGGCTGCCTTCTTCTTGGGCTTTCTCTCTGCCTTGACTCATACAAGTGCCATGATG | |
| AAACTGTCCTTTTGCAAATCCCACATTATCAACCATTACTTCTGTGATGTTCTTCCCCTCCT | |
| CAATCTCTCCTGCTCCAACACACACCTCAATGAGCTTCTACTTTTTATCATTGCGGGGTTTA | |
| ACACCTTGGTGCCCACCCTAGCTGTTGCTGTCTCCTATGCCTTCATCCTCTACAGCATCCTT | |
| CACATCCGCTCCTCAGAGGGCCGGTCCAAAGCTTTTGGAACATGCAGCTCTCATCTCATGG | |
| CTGTGGTGATCTTCTTTGGGTCCATTACCTTCATGTATTTCAAGCCCCCTTCAAGTAACTCC | |
| CTGGACCAGGAGAAGGTGTCCTCTGTGTTCTACACCACGGTGATCCCCATGCTGAACCCTT | |
| TAATATACAGTCTGAGGAATAAGGATGTGAAGAAAGCATTAAGGAAGGTCTTAGTAGGAA | |
| AATGA | |
| AOLFR137 sequences: | |
| MSPENQSSVSEFLLLGLPIRPEQQAVFFALFLGMYLTTVLGNLLIMLLIQLDSHLHTPMTTFLSH | (SEQ ID NO:253) |
| LALTDISFSSVTVPKMLMNMQTQHLAVFYKGCISQTTTFIFFADLDSFLITSMAYDRYVAICHPL | |
| HYATIMTQSQCVMLVAGSWVIACACALLHTLLLAQLSFCADHIIPHTTCDLGALLKLSCSDTSL | |
| NQLAIFTAALTAIMLPFLCILVSYGHIGVTILQIPSTKGICKALSTCGSHLSVVTIYYRTIIGLTTLP | |
| PSSNTNDKNIIASVIYTAVTPMLNPFIYSLRNKDIKGALRKLLSRSGAVAHACNLSTLGG | |
| ATGAGCCCTGAGAACCAGAGCAGCGTGTCCGAGTTCCTCCTCCTGGGCCTCCCCATCCGGC | (SEQ ID NO:254) |
| CAGAGCAGCAGGCCGTGTTCTTCGCCCTGTTCCTGGGCATGTACCTGACCACGGTGCTGGG | |
| GAACCTGCTCATCATGCTGCTCATCCAGCTAGACTCTCACCTTCACACCCCCATGTACTTCT | |
| TCCTTAGCCACTTGGCCCTCACTGACATCTCCTTTTCATCTGTCACTGTCCCTAAGATGCTG | |
| ATGAACATGCAGACTCAGCACCTAGCCGTCTTTTACAAGGGATGCATTTCACAGACATATT | |
| TTTTCATATTTTTTGCTGACTTAGACAGTTTCCTTATCACTTCAATGGCATATGACAGGTAT | |
| GTGGCCATCTGTCATCCTCTACATTATGCCACCATCATGACTCAGAGCCAGTGTGTCATGC | |
| TGGTGGCTGGGTCCTGGGTCATCGCTTGTGCGTGTGCTCTTTTGCATACCCTCCTCCTGGCC | |
| CAGCTTTCCTTCTGTGCTGACCACATCATCCCTCACTACTTCTGTGACCTTGGTGCCCTGCT | |
| CAAGTTGTCCTGCTCAGACACCTCCCTCAATCAGTTAGCAATCTTTACAGCAGCATTGACA | |
| GCCATTATGCTTCCATTCCTGTGCATCCTGGTTTCTTATGGTCACATTGGGGTCACCATCCT | |
| CCAGATTCCCTCTACCAAGGGCATATGCAAAGCCTTGTCCACTTGTGGATCCCACCTCTCA | |
| GTGGTGACTATCTATTATCGGACAATTATTGGTCTCTATTTTCTTCCCCCATCCAGCAACAC | |
| CAATGACAAGAACATAATTGCTTCAGTGATATACACAGCAGTCACTCCCATGTTGAACCCA | |
| TTCATTTACAGTCTGAGAAATAAAGACATTAAGGGAGCCCTAAGAAAACTCTTGAGTAGG | |
| TCAGGCGCAGTGGCTCATGCCTGTAATCTCAGCACTTTGGGAGGCTGA | |
| AOLFR138 sequences: | |
| MLNFTDVTEFILLGLTSRREWQVLFFIVFLVVYIITVVGNIGMMLLIKVSPQLNSPMTTFLSHLS | (SEQ ID NO:255) |
| FVDVWFSSNVTPKMLENLFSDKKTISYADCLAQCFFFIALVHVEIFILAAIAFDRYTVIGNPLLY | |
| GSKMSRGVCIRLITFPYIYGFLTSLTATLWTYGLTTCGKIEINHFYCADPPLIKMACAGTFVKEY | |
| TMLILAGINFTYSLTVIIISYLFILIAILRMRSAEGRQKAFSTCGSHPTAVIIFYGTLIFMYLRRPTE | |
| ESVEQGKMVAVFYTTVIPMLNPMIYSLRNKDVKKAMMKVISRSC | |
| ATGCTCAATTTCACCGATGTGACAGAGTTCATTCTTTTGGGGCTAACGAGCCGTCGGGAAT | (SEQ ID NO:256) |
| GGCAAGTTCTCTTCTTCATCGTTTTTCTTGTGGTCTACATTATCACCGTGGTGGGCAATATC | |
| GGCATGATGTTGTTAATCAAGGTCAGTCCTCAGCTTAACAGCCCCATGTACTTTTTCCTCA | |
| GTCACTTGTCATTTGTTGATGTGTGGTTTTCTTCCAATGTCACCCCTAAAATGTTGGaAAAT | |
| CTGTTATCAGATAAAAAAACAATTTCTTATGCTGGCTGTTTAGCACAGTGTTTCTTCTTCAT | |
| TGCTCTTGTCCATGTGGAAATTTTTATTCTTGCTGCGATTGCCTTTGATAGATACACAGTGA | |
| TTGGAAATCCTTTGCTTTATGGCAGCAAAATGTCAAGGGATGTCTGTATTCGACTGATTAC | |
| TTTCCCTTACATTTATGGTTTTCTGACGAGTCTGACAGCAACATTATGGACTTATGGCTTGT | |
| ACTTCTGTGGAAAAATTGAGATCAACCATTTCTACTGTGCAGATCCACCTCTCATCAAAAT | |
| GGCCTGTGCCGGGACCTTTGTAAAAGAATATACAATGCTCATACTTGCCGGCATCAACTTC | |
| ACATATTCCCTGACTGTAATTATCATCTCTTACTTATTCATCCTCATTGCCATTCTGCGAAT | |
| GCGCTCAGCAGAAGGAAGGCAGAAGGCCTTTTCCACATGTGGGTCCCATCTGACAGCTGT | |
| CATCATATTCTATGGTACTCTGATCTTCATGTATCTCAGACGTCCCACAGAGGAGTCTGTG | |
| GAGCAGGGGAAGATGGTGGCTGTGTTCTATACCACAGTGATCCCCATGTTGAATCCCATGA | |
| TCTACAGTCTGAGGAACAAGGATGTGAAAAAGGCCATGATGAAAGTGATCAGCAGATCAT | |
| GTTAA | |
| AOLFR139 sequences: | |
| MGFPGIHSWQHWLSLPLALLYLLALSANILILIIINKEAALHQPMYTTLGILAMADIGLATTIMP | (SEQ ID NO:257) |
| KILAILWFNAKTISLLECFAQMYAIHCFVAMESSTFVCMAIDRYVAICRPLRYPSIITESFVFKAN | |
| GFMALRNSLCLISVPLLAAQRHYCSQNQIEHCLCSNLGVTSLSCDDRRINSINQVLLAWTLMGS | |
| DLGLIILSYALILYSVLKLNSPEAASKALSTCTSHLILILFFYTVIIVISITRSTGMRVPLIPVLLNVL | |
| HNVIPPALNPMVYALKNKELRQGLYKVLRLGVKGT | |
| ATGGGATTCCCTGGCATTCACAGTTGGCAGCACTGGCTCTCCCTGCCCCTGGCTCTGCTCT | |
| ACCTCTTAGCTCTCAGTGCCAACATCCTTATCCTGATCATCATCAACAAAGAGGCAGCACT | |
| GCACCAGCCTATGTACTATTTCCTGGGCATCTTGGCTATGGCAGACATAGGCCTGGCTACC | |
| ACCATCATGCCTAAGATTTTGGCCATCTTATGGTTCAATGCTAAGACCATCAGTCTCCTGG | |
| AGTGCTTTGCTCAGATGTATGCCATACATTGCTTTGTGGCCATGGAATCAAGTACCTTTGT | |
| CTGCATGGCTATTGATAGATATGTAGCCATTTGTCGACCGCTACGATATCCATCAATCATC | |
| ACTGAATCTTTTGTTTTCAAAGCAAATGGGTTCATGGCACTGAGAAACAGCCTGTGTCTCA | |
| TCTCAGTGCCTCTGTTGGCTGCCCAGAGGCATTACTGCTCCCAGAATCAAATTGAGCACTG | |
| TCTTTGTTCTAACCTTGGAGTCACTAGCCTATCTTGTGATGATCGAAGAATCAATAGCATT | |
| AACCAGGTCCTTTTGGCTTGGACACTCATGGGAAGTGACCTGGGTTTGATTATTTTATCAT | |
| ATGCTCTAATACTTTACTCTGTCCTGAAGCTGAACTCTCCAGAAGCTGCATCCAAGGCCTT | |
| AAGTACCTGCACCTCCCACCTCATCTTAATCCTTTTCTTCTACACAGTCATCATTGTGATTT | |
| CCATTACTCGTAGTACAGGAATGAGAGTTCCCCTTATTCCAGTTCTACTTAATGTGCTACA | |
| CAATGTCATTCCCCCTGCCCTGAACCCCATGGTATATGCACTCAAGAACAAGGAACTCAGG | |
| CAAGGCTTATACAAGGTACTTAGACTGGGAGTGAAGGGCACCTGA | |
| AOLFR140 sequences: | |
| MLTLNKTDLIPASFILNGVPGLEDTQLWISFPFCSMYVVAMVGNCGLLYLIHYEDALHKPMYY | (SEQ ID NO:259) |
| FLAMLSFTDLVMCSSTIPKALCIFWFHLKDIGFDECLVQMFFIHTFTGMESGVLMLMALDRYV | |
| AICYIPLRYSTILTNPVIAKVGTATFLRGVLLIIPFTFLTKRLPYCRGNILPHTYCDHMSVAKLSCG | |
| NVKVNAIYGLMVALLIGGFDILCITISYTMILRAVVSLSSADARQKAFNTCTAHICAIVFSYTPAF | |
| FSFFSHRFGEHIIPPSCHIIVANIYLLLPPTMNPIVYGVKTKQIRDCVIRILSGSKDTKSYSM | |
| ATGCTAACACTGAATAAAACAGACCTAATACCAGCTTCATTTATTCTGAATGGAGTCCCAG | (SEQ ID NO:260) |
| GACTGGAAGACACACAACTCTGGATTTCCTTCCCATTCTGCTCTATGTATGTTGTGGCTAT | |
| GGTAGGGAATTGTGGACTCCTCTACCTCATTCACTATGAGGATGCCCTGCACAAACCCATG | |
| TACTACTTCTTGGCCATGCTTTCCTTTACTGACCTTGTTATGTGCTCTAGTACAATCCCTAA | |
| AGCCCTCTGCATCTTCTGGTTTCATCTCAAGGACATTGGATTTGATGAATGCCTTGTCCAG | |
| ATGTTCTTCATCCACACCTTCACAGGGATGGAGTCTGGGGTGCTTATGCTTATGGCCCTGG | |
| ATCGCTATGTGGCCATCTGCTACCCCTTACGCTATTCAACTATCCTCACCAATCCTGTAATT | |
| GCAAAGGTTGGGACTGCCACCTTCCTGAGAGGGGTATTACTCATTATTCCCTTTACTTTCCT | |
| CACCAAGCGCCTGCCCTACTGCAGAGGCAATATACTTCCCCATACCTACTGTGACCACATG | |
| TCTGTAGCCAAATTGTCCTGTGGTAATGTCAAGGTCAATGCCATCTATGGTCTGATGGTTG | |
| CCCTCCTGATTGGGGGCTTTGACATACTGTGTATCACCATCTCCTATACCATGATTCTCCGG | |
| GCAGTGGTCAGCCTCTCCTCAGCAGATGCTCGGCAGAAGGCCTTTAATACCTGCACTGCCC | |
| ACATTTGTGCCATTGTTTTCTCCTATACTCCAGCTTTCTTCTCCTTCTTTTCCCACCGCTTTG | |
| GGGAACACATAATCCCCCCTTCTTGCCACATCATTGTAGCCAATATTTATCTGCTCCTACCA | |
| CCCACTATGAACCCTATTGTCTATGGGGTGAAAACCAAACAGATACGAGACTGTGTCATAA | |
| GGATCCTTTCAGGTTCTAAGGATACCAAATCCTACAGCATGTGA | |
| AOLFR141 sequences: | |
| MSSTLGHNMESPNHTDVDPSVFFLLGIPGLEQFHLWLSLPVCGLGTATIVGNITILVVVATEPVL | (SEQ ID NO:261) |
| HKPVYLFLCMLSTIDLAASVSTVPKLLAIFWCGAGHISASACLAQMFFIHAFCMMESTVLLAM | |
| AFDRYVAICHPLRYATILTDTIIAHIGVAAVVRGSLLMLPCPFLIGRLNFCQSHVILHTYCEHMA | |
| VVKLACGDTRPNRVYGLTAALLVIGVDLFCIGLSYALSAQAVLRLSSHEARSKALGTCGSHVC | |
| VILISYTPALFSFFTHRFGHHVPVHIHILLANVYLLLPPALNPVVYGVKTKQIRKRVVRVFQSGQ | |
| GMGIKASE | |
| ATGTCCAGCACTCTTGGCCACAACATGGAATCTCCTAATCACACTGATGTTGACCCTTCTG | (SEQ ID NO:262) |
| TCTTCTTCCTCCTGGGCATCCCAGGTCTGGAACAATTTCATTTGTGGCTCTCACTCCCTGTG | |
| TGTGGCTTAGGCACAGCCACAATTGTGGGCAATATAACTATTCTGGTTGTTGTTGCCACTG | |
| AACCAGTCTTGCACAAGCCTGTGTACCTTTTTCTGTGCATGCTCTCAACCATCGACTTGGCT | |
| GCCTCTGTCTCCACAGTTCCCAAGCTACTGGCTATCTTCTGGTGTGGAGCCGGACATATAT | |
| CTGCCTCTGCCTGCCTGGCACAGATGTTCTTCATTCATGCCTTCTGCATGATGGAGTCCACT | |
| GTGCTACTGGCCATGGCCTTTGATCGCTACGTGGCCATCTGCCACCCACTCCGCTATGCCA | |
| CAATCCTCACTGACACCATCATTGCCCACATAGGGGTGGCAGCTGTAGTGCGAGGCTCCCT | |
| GCTCATGCTCCCATGTCCCTTCCTTATTGGGCGTTTGAACTTCTGCCAAAGCCATGTGATCC | |
| TACACACGTACTGTGAGCACATGGCTGTGGTGAAGCTGGCCTGTGGAGACACCAGGCCTA | |
| ACCGTGTGTATGGGCTGACAGCTGCACTGTTGGTCATTGGGGTTGACTTGTTTTGCATTGG | |
| TCTCTCCTATGCCCTAAGTGCACAAGCTGTCCTTCGCCTCTCATCCCATGAAGCTCGGTCCA | |
| AGGCCCTAGGGACCTGTGGTTCCCATGTCTGTGTCATCCTCATCTCTTATACACCAGCCCTC | |
| TTCTCCTTTTTTACACACCGCTTTGGCCATCACGTTCCAGTCCATATTCACATTCTTTTGGC | |
| CAATGTTTATCTGCTTTTGCCACCTGCTCTTAATCCTGTGGTATATGGAGTTAAGACCAAAC | |
| AGATCCGTAAAAGAGTTGTCAGGGTGTTTCAAAGTGGGCAGGGAATGGGCATCAAGGCAT | |
| CTGAGTGA | |
| AOLFR143 sequences: | |
| MLGLNGTPFQPATLQLTGIPGIQTGLTWVALIFCILYMISIVGNLSILTLVFWEPALHQPMYTTL | (SEQ ID NO:263) |
| SMLALNDLGVSFSTLPTVISTFCFNYNHVAFNACLVQMFFIHTFSFMESGILLAMSLDRFVAICY | |
| PLRYVTVLTHNRILAMGLGILTKSFTTLFPFPFVVKRLPFCKGNVLHHSYCLHPDLMKVACGDI | |
| HVNNIYGLLVIIFTYGMDSTFILLSYALILRAMLVIISQEQRLKALNTCMSHICAVLAFYVPIIAVS | |
| MIHRFWKSAPPVVHVMMSNVYLFVPPMLNPIIYSVKTKEIRKGILKFFHKSQA | |
| ATGCTGGGTCTCAATGGCACCCCCTTCCAGCCAGCAACACTCCAGCTGACAGGCATTCCTG | (SEQ ID NO:264) |
| GGATACAAACAGGCCTCACCTGGGTTGCCCTGATTTTCTGCATCCTCTACATGATCTCCATT | |
| GTAGGTAACCTCAGCATTCTCACTCTGGTGTTTTGGGAGCCTGCTCTGCATCAGCCCATGT | |
| ACTACTTCCTCTCTATGCTCGCTCTCAATGATCTGGGAGTGTCCTTTTCTACACTTCCCACT | |
| GTGATTTCTACTTTCTGCTTCAACTACAACCATGTTGCGTTTAATGCTTGCCTGGTCCAGAT | |
| GTTCTTCATCCACACTTTCTCCTTCATGGAGTCAGGCATACTGCTGGCCATGAGCTTGGATC | |
| GCTTTGTGGCTATTTGTTATCCATTACGCTATGTCACTGTGCTCACTCACAACCGTATATTG | |
| GCTATGGGTCTGGGCATCCTTACCAAGAGTTTCACCACTCTCTTCCCTTTCCCTTTTGTGGT | |
| GAAACGACTGCCCTTCTGCAAAGGCAATGTTTTGCATCACTCCTACTGTCTCCATCCAGAT | |
| CTCATGAAAGTAGCATGTGGAGACATCCATGTTAACAACATTTATGGGCTCTTGGTGATCA | |
| TTTTTACCTATGGTATGGACTCAACTTTCATCCTGCTTTCCTACGCATTGATCCTGAGAGCC | |
| ATGCTGGTCATCATATCCCAGGAACAGCGGCTCAAGGCACTCAACACCTGCATGTCACACA | |
| TCTGTGCAGTGCTGGCCTTTTATGTGCCCATAATTGCTGTCTCCATGATTCACCGCTTCTGG | |
| AAAAGTGCTCCACCTGTTGTTCATGTCATGATGTCCAATGTCTACCTGTTTGTACCACCCAT | |
| GCTCAACCCTATCATCTACAGTGTGAAAACCAAGGAGATCCGCAAAGGGATTCTCAAGTTC | |
| TTCCATAAATCCCAGGCCTGA | |
| AOLFR144 sequences: | |
| MGLFNVTHPAFFLLTGIPGLESSHSWLSGPLCVMYAVALGGNTVILQAVRVEPSLHEPMYTTL | (SEQ ID NO:265) |
| SMLSFSDVAISMATLPTVLRTFCLNARNITFDACLIQMFLIHFFSMMESGILLAMSFDRYVAICD | |
| PLRYATVLTTEVIAAMGLGAAARSFITLFPLPFLIKRLPICRSNVLSHSYCLHPDMMRLACADISI | |
| NSIYGLFVLVSTFGMDLFFIFLSYVLILRSVMATASREERLKALNTCVSHILAVLAPYVPMIGVS | |
| TVHRFGKHVPCYIHVLMSNVYLFVPPVLNPLIYSAKTKEIRRAIFRMFHHIKI | |
| ATGGGGTTGTTCAATGTCACTCACCCTGCATTCTTCCTGCTGACTGGTATCCCTGGTCTGGA | (SEQ ID NO:266) |
| GAGCTCTCACTCCTGGCTGTCAGGGCCCCTCTGCGTGATGTATGCTGTGGCCCTTGGGGGA | |
| AATACAGTGATCCTGCAGGCTGTGCGAGTGGAGCCCAGCCTCCATGAGCCCATGTACTACT | |
| TCCTGTCCATGTTGTCCTTCAGTGATGTGGCCATATCCATGGCCACACTGCCCACTGTACTC | |
| CGAACCTTCTGCCTCAATGCCCGCAACATCACTTTTGATGCCTGTCTAATTCAGATGTTTCT | |
| TATTCACTTCTTCTCCATGATGGAATCAGGTATTCTGCTGGCCATGAGTTTTGACCGCTATG | |
| TGGCCATTTGTGACCCCTTGCGCTATGCAACTGTGCTCACCACTGAAGTCATTGCTGCAAT | |
| GGGTTTAGGTGCAGCTGCTCGAAGCTTCATCACCCTTTTCCCTCTTCCCTTTCTTATTAAGA | |
| GGCTGCCTATCTGCAGATCCAATGTTCTTTCTCACTCCTACTGCCTGCACCCAGACATGATG | |
| AGGCTTGCCTGTGCTGATATCAGTATCAACAGCATCTATGGACTCTTTGTTCTTGTATCCAC | |
| CTTTGGCATGGACCTGTTTTTTATCTTCCTCTCCTATGTGCTCATTCTGCGTTCTGTCATGG | |
| CCACTGCTTCCCGTGAGGAACGCCTCAAAGCTCTCAACACATGTGTGTCACATATCCTGGC | |
| TGTACTTGCATTTTATGTGCCAATGATTGGGGTCTCCACAGTGCACCGCTTTGGGAAGCAT | |
| GTCCCATGCTACATACATGTCCTCATGTCAAATGTGTACCTATTTGTGCCTCCTGTGCTCAA | |
| CCCTCTCATTTATAGCGCCAAGACAAAGGAAATCCGCCGAGCCATTTTCCGCATGTTTCAC | |
| CACATCAAAATATGA | |
| AOLFR145 sequences: | |
| MSVQYSLSPQFMLLSNITQFSPIFYLTSFPGLEGIKHWIFIPFFFMYMVAISGNCFILIIIKTNPRLH | (SEQ ID NO:267) |
| TPMYYLLSLLALTDLGLCVSTLPTTMGIFWFNSQSITTGACQIQMFCIHSFSFMESSVLLMMSFD | |
| RFVAICHPLRYSVIITGQQVVRAGLIVIFRGPVATIPIVLLLKAFPYCGSVVLSHSFCLHQEVIQLA | |
| CTDTTFNNLYGLMVVVFTVMLDLVLIALSYGLILHTVAGLASQEEQRRAFQTCTAHLCAVLVF | |
| FVPMMGLSLVHRFGKHAPPAIHLLMANVYLFVPPMLNPIIYSIKTKEIHRAIIKLLGLKIKASK | |
| ATGTCAGTCCAATATTCGCTCAGTCCTCAATTCATGCTGCTATCCAACATTACTCAGTTTAG | (SEQ ID NO:268 |
| CCCCATATTCTATCTCACCAGCTTTCCTGGATTGGAAGGCATCAAACACTGGATTTTCATCC | |
| CCTTTTTCTTTATGTACATGGTTGCCATCTCAGGCAATTGTTTCATTCTGATCATTATTAAG | |
| ACCAACCCTCGTCTGCACACACCCATGTACTATCTACTATCCTTGCTGGCCCTCACTGACCT | |
| GGGGCTGTGTGTGTCCACGTTGCCCACCACTATGGGGATCTTCTGGTTTAACTCCCAGAGT | |
| ATCTACTTTGGAGCGTGTCAAATCCAGATGTTCTGCATCCACTCTTTTTCCTTCATGGAGTC | |
| CTCAGTGCTCCTCATGATGTCCTTTGACCGCTTTGTGGCCATCTGCCACCCTCTGAGGTATT | |
| CGGTCATTATCACTGGCCAGCAAGTGGTCAGAGCAGGCCTAATTGTCATCTTCCGGGGACC | |
| TGTGGCCACTATCCCTATTGTCCTCCTCCTGAAGGCTTTTCCCTACTGTGGATCTGTGGTCC | |
| TCTCCCACTCATTTTGCCTGCACCAGGAAGTGATACAGCTGGCCTGCACAGATACCACCTT | |
| CAATAATCTGTATGGACTGATGGTGGTAGTTTTCACTGTGATGCTGGACCTGGTGCTCATC | |
| GCACTGTCCTATGGACTCATCCTGCACACAGTAGCAGGCCTGGCCTCCCAAGAGGAGCAGC | |
| GCCGTGCCTTTCAGACATGCACCGCTCATCTCTGTGCTGTGCTAGTATTCTTTGTGCCCATG | |
| ATGGGGCTGTCCCTGGTGCACCGTTTTGGGAAGCATGCCCCACCTGCTATTCATCTTCTTAT | |
| GGCCAATGTCTACCTTTTTGTGCCTCCCATGCTTAACCCAATCATATACAGCATTAAGACC | |
| AAGGAGATCCACCGTGCCATTATCAAACTCCTAGGTCTTAAAAAGGCCAGTAAATGA | |
| AOLFR146 sequences: | |
| MSQVTNTTQEGITTILTDIPGFEASHIWISIPVCCLYTISIMGNTTILTVIRTEPSVHQRMYLFLSM | (SEQ ID NO:269) |
| LALTDLGLTLTTLPTVMQLLWFNVRRISSEACFAQFFFLHGFSFMESSVLLAMSVDCYVAICCP | |
| LHYASILTNEVIGRTGLAIICCCVLAVLPSLFLLKRLPFCHSHLLSRSYCLHQDMIRLVCADIRLN | |
| SWYGFALALLIIIVDPLLIVISYTLILKNILGTATWAERLRALNNCLSHILAVLVLYIPMVGVSMT | |
| HRFAKHASPLVHVIMANIYLLAPPVMNPIIYSVKNKQIQWGMLNThSLKNMHSR | |
| ATGTCCCAGGTGACTAACACCACACAAGAAGGCATCTACTTCATCCTCACGGACATCCCTG | (SEQ ID NO:270) |
| GATTTGAGGCCTCCCACATCTGGATCTCCATCCCCGTCTGCTGTCTCTACACCATCTCCATC | |
| ATGGGCAATACCACCATCCTCACTGTCATTCGCACAGAGCCATCTGTCCACCAGCGCATGT | |
| ATCTGTTTCTCTCCATGCTGGCCCTGACGGACCTGGGTCTCACCCTCACCACCCTACCCACA | |
| GTCATGCAGCTTCTCTGGTTCAACGTTCGTAGAATCAGCTCTGAGGCCTGTTTTGCTCAGTT | |
| TTTCTTCCTTCATGGATTCTCCTTTATGGAGTCTTCTGTCCTCCTGGCTATGTCCGTTGACT | |
| GCTATGTGGCCATCTGCTGTCCCCTCCATTATGCCTCCATCCTCACCAATGAAGTCATTGGT | |
| AGAACTGGGTTAGCCATCATTTGCTGCTGTGTTCTGGCGGTTCTTCCCTCCCTTTTCTTACT | |
| CAAGCGACTGCCTTTCTGCCACTCCCACCTTCTCTCTCGCTCCTATTGCCTCCACCAGGATA | |
| TGATCCGCCTGGTCTGTGCTGACATCAGGCTCAACAGCTGGTATGGATTTGCTCTTGCCTT | |
| GCTCATTATTATCGTGGATCCTCTGCTCATTGTGATCTCCTATACACTTATTCTGAAAAATA | |
| TCTTGGGCACAGCCACCTGGGCTGAGCGACTCCGTGCCCTCAATAACTGCCTGTCCCACAT | |
| TCTAGCTGTCCTGGTCCTCTACATTCCCATGGTTGGTGTATCTATGACTCATCGCTTTGCCA | |
| AGCATGCCTCTCCACTGGTCCATGTTATCATGGCCAATATCTACCTGCTGGCACCCCCGGT | |
| GATGAACCCCATCATTTACAGTGTAAAGAACAAGCAGATCCAATGGGGAATGTTAAATTTC | |
| CTTTCCCTCAAAAATATGCATTCAAGATGA | |
| AOLFR147 sequences: | |
| MPSASAMIIFNLSSYNPGPFILVGIPGLEQFHVWIGIPFCIIYIVAVVGNCILLYLIVVEHSLHEPMF | (SEQ ID NO:271) |
| FFLSMLAMTDLILSTAGVPKALSIFWLGAREITFPGCLTQMFFLHYNFVLDSAILMAMAFDHYV | |
| AICSPLRYTTILTPKTIIKSAMGISFRSFCIILPDVFLLTCLPFCRTRIIPHTYCEHIGVAQLACADISI | |
| NFWYGFCVPIMTVISDVILIAVSYAHILCAVFGLPSQDACQKALGTCGSHVCVILMFYTPAFFSI | |
| LAHRFGHNVSRTFHIMFANLYIVIPPALNPMVYGVKTKQIRDKVILLFSKGTG | |
| ATGCCATCTGCCTCTGCCATGATCATTTTCAACCTGAGCAGTTACAATCCAGGACCCTTCAT | (SEQ ID NO:272) |
| TCTGGTAGGGATCCCAGGCCTGGAGCAATTCCATGTGTGGATTGGAATTCCCTTCTGTATC | |
| ATCTACATTGTAGCTGTTGTGGGAAACTGCATCCTTCTCTACCTCATTGTGGTGGAGCATA | |
| GTCTTCATGAACCCATGTTCTTCTTTCTCTCCATGCTGGCCATGACTGACCTCATCTTGTCC | |
| ACAGCTGGTGTGCCTAAAGCACTCAGTATCTTTTGGCTAGGGGCTCGCGAAATCACATTCC | |
| CAGGATGCCTTACACAAATGTTCTTCCTTCACTATAACTTTGTCCTGGATTCAGCCATTCTG | |
| ATGGCCATGGCATTTGATCACTATGTAGCTATCTGTTCTCCCTTGAGATATACCACCATCTT | |
| GACTCCCAAGACCATCATCAAGAGTGCTATGGGCATCTCCTTTCGAAGCTTCTGCATCATC | |
| CTGCCAGATGTATTCTTGCTGACATGCCTGCCTTTCTGCAGGACACGCATCATACCCCACA | |
| CATACTGTGAGCATATAGGTGTTGCCCAGCTCGCCTGTGCTGATATCTCCATCAACTTCTG | |
| GTATGGCTTTTGTGTTCCCATCATGACGGTCATCTCAGATGTGATTCTCATTGCTGTTTCCT | |
| ACGCACACATCCTCTGTGCTGTCTTTGGCCTTCCCTCCCAAGATGCCTGGCAGAAAGCCCT | |
| CGGCACTTGTGGTTCTCATGTCTGTGTCATCCTCATGTTTTATACACCTGCCTTTTTCTCCA | |
| TCCTCGCCCATCGCTTTGGACACAATGTCTCTCGCACCTTCCACATCATGTTTGCCAATCTC | |
| TACATTGTTATCCCACCTGCACTCAACCCCATGGTTTACGGAGTGAAGACCAAGCAGATCA | |
| GAGATAAGGTTATACTTTTGTTTTCTAAGGGTACAGGATGA | |
| AOLFR148 sequences: | |
| MPTVNHSGTSHTVFHLLGIPGLQDQHMWISIPFFISYVTALLGNSLLIFIILTKRSLHEPMYLFLC | (SEQ ID NO:273) |
| MLAGADIVLSTCTIPQALAIFWFRAGDISLDRCITQLFFIHSTFISESGILLVMAFDHYIAICYPLR | |
| YTTILTNALIKKICVTVSLRSYGTIFPIIFLLKRLTFCQNNIIPHTFCEHIGLAKYACNDIRINIWYG | |
| FSILMSTVVLDVVLIFISYMLILHAVFHMPSPDACHKALNTFGSHVCIIILFYGSGIFTILTQRFGR | |
| HIPPCIHIPLANVCILAPPMLNPIIYGIKTKQIQEQVVQFLFIKQKITLV | |
| ATGCCTACTGTAAACCACAGTGGCACTAGCCACACAGTCTTCCACTTGCTGGGCATCCCTG | (SEQ ID NO:274) |
| GCCTACAGGACCAGCACATGTGGATTTCTATCCCATTCTTCATTTCCTATGTCACCGCCCTT | |
| CTTGGGAACAGCCTGCTCATCTTCATTATCCTCACAAAGCGCAGCCTCCATGAACCCATGT | |
| ACCTCTTCCTCTGCATGCTGGCTGGAGCAGACATTGTCCTCTCCACGTGCACCATTCCTCAG | |
| GCCTTAGCTATCTTCTGGTTCCGTGCTGGGGACATGTCCCTGGATCGTTGCATCACTCAGCT | |
| CTTCTTCATCCATTCCACCTTCATCTCTGAGTCAGGGATCTTGCTGGTGATGGCCTTTGACC | |
| ACTATATTGCCATATGCTACCCACTGAGGTACACCACCATTCTTACAAATGCTCTGATCAA | |
| GAAAATTTGTGTGACTGTCTCTCTGAGAAGTTATGGTACAATTTTCCCTATCATATTTCTTT | |
| TAAAAAGATTGACTTTCTGCCAGAATAATATTATTCCACACACCTTTTGTGAACACATTGG | |
| CCTAGCCAAATATGCATGTAATGACATTCGAATAAACATTTGGTATGGGTTTTCCATTCTA | |
| ATGTCGACGGTGGTCTTAGATGTTGTACTAATTTTTATTTCCTATATGCTGATTCTCCATGC | |
| TGTCTTCCACATGCCTTCTCCAGATGCTTGCCACAAAGCTCTCAACACATTTGGCTCCCATG | |
| TCTGCATCATCATCCTCTTTTATGGGTCTGGCATCTTCACAATCCTTACCCAGAGGTTTGGA | |
| CGCCACATTCCACCTTGTATCCACATCCCGTTGGCTAATGTCTGCATTCTGGCTCCACCTAT | |
| GCTGAATCCCATTATTTATGGGATCAAAACCAAGCAAATCCAGGAACAGGTGGTTCAGTTT | |
| TTGTTTATAAAACAGAAAATAACTTTGGTTTAA | |
| AOLFR149 sequences: | |
| MSNASLLTAFILMGLPHAPALDAPLFGVFLVVYVLTVLGNLLILLVIRVDSHLHTTMYTTLTNL | (SEQ ID NO:275) |
| SFIDMWFSTVTVPKLLMTLVFPSGRAISFHSCMAQLTTFHFLGGTECFLYRVMSCDRYLAISYP | |
| LRYTSMMTGRSCTLLATSTWLSGSLHSAVQAILTFHLPYCGPNWIQHYLCDAPPILKLACADTS | |
| AIETVIFVTVGIVASGCFVLIVLSYVSIVCSILRIRTSEGKHRAFQTCASHCIVVLCFFGPGLFIYLR | |
| PGSRKAVDGVVAVFYTVLTPLLNPVVYTLRNKEVKKALLKLKDKVAHSQSK | |
| ATGTCCAACGCCAGCCTACTGACAGCGTTCATCCTCATGGGCCTTCCCCATGCCCCAGCGC | (SEQ ID NO:276) |
| TGGACGCCCCCCTCTTTGGAGTCTTCCTGGTGGTTTACGTGCTCACTGTGCTGGGGAACCT | |
| CCTCATCCTGCTGGTGATCAGGGTGGATTCTCACCTCCACACCACCATGTACTACTTCCTCA | |
| CCAACCTGTCGTTCATTGACATGTGGTTCTCCACTGTCACGGTGCCCAAATTGCTGATGAC | |
| TTTGGTGTTCCCAAGTGGCAGGGCTATCTCCTTCCACAGCTGCATGGCTCAGCTCTATTTCT | |
| TTCACTTCCTAGGGGGCACCGAGTGTTTCCTCTACAGGGTCATGTCCTGTGATCGCTACCT | |
| GGCCATCAGTTACCCGCTCAGGTACACCAGCATGATGACTGGGCGCTCGTGTACTCTTCTG | |
| GCCACCAGCACTTGGCTCAGTGGCTCTCTGCACTCTGCTGTCCAGGCCATATTGACTTTCC | |
| ATTTGCCCTACTGTGGACCCAACTGGATCCAGCACTATTTGTGTGATGCACCGCCCATCCT | |
| GAAACTGGCCTGTGCAGACACCTCAGCCATAGAGACTGTCATTTTTGTGACTGTTGGAATA | |
| GTGGCCTCGGGCTGCTTTGTCCTGATAGTGCTGTCCTATGTGTCCATCGTCTGTTCCATCCT | |
| GCGGATCCGCACCTCAGAGGGGAAGCACAGAGCCTTTCAGACCTGTGCCTCCCACTGTATC | |
| GTGGTCCTTTGCTTCTTTGGCCCTGGTCTTTTCATTTACCTGAGGCCAGGCTCCAGGAAAGC | |
| TGTGGATGGAGTTGTGGCCGTTTTCTACACTGTGCTGACGCCCCTTCTCAACCCTGTTGTGT | |
| ACACCCTGAGGAACAAGGAGGTGAAGAAAGCTCTGTTGAAGCTGAAAGACAAAGTAGCAC | |
| ATTCTCAGAGCAAATAG | |
| AOLFR150 sequences: | |
| MELGNVTRVKEFIFLGLTQSQDQSLVLFLFLCLVYMTTLLGNLLIMVTVTCESRLHTPMTTLLR | (SEQ ID NO:277) |
| NLAILDICFSSTTAPKVLLDLLSKKKTISYTSCMTQIFLFHLLGGADIFSLSVMAFDCYMAISKPL | |
| HYVTIMSRGQCTALISASWMGGFVHSIVQISLLLPLPFCGPNVLDTFYCDVPQVLKLTCTDTFA | |
| LEFLMISNNGLVTTLWFIFLLVSYTVILMTLRSQAGGGRRKAISTCTSPHHCGDPAFCALHLCLC | |
| PALHCPPHRKGHLCHLHCHLPSAEPFDLHSEEPGNEVSHEKTEEKTRAF | |
| ATGGAGTTGGGAAATGTCACCAGAGTAAAAGAATTTATATTTCTGGGACTTACTCAATCCC | (SEQ ID NO:278) |
| AAGACCAGAGTTTGGTCTTGTTTCTTTTTTTATGTCTTGTGTACATGACGACTCTGCTGGGA | |
| AACCTCCTCATCATGGTCACCGTGACCTGTGAGTCTCGCCTTCACACCCCCATGTACTTCCT | |
| GCTCCGCAATCTAGCCATCCTTGACATCTGCTTCTCCTCCACAACTGCTCCTAAAGTCTTGC | |
| TGGACCTTCTGTCAAAGAAAAAGACCATATCCTATACAAGCTGCATGACACAGATATTTCT | |
| CTTCCACCTCCTTGGTGGGGCAGACATTTTTTCTCTCTCTGTGATGGCGTTTGACTGCTACA | |
| TGGCCATCTCCAAGCCCCTGCACTATGTGACCATCATGAGTAGAGGGCAATGCACTGCCCT | |
| CATCTCTGCCTCTTGGATGGGGGGCTTTGTCCACTCCATCGTGCAGATCTCCCTGTTGCTGC | |
| CTCTCCCTTTCTGTGGACCCAATGTTCTTGACACTTTCTACTGCGATGTCCCCCAGGTCCTC | |
| AAACTCACTTGCACTGACACTTTTGCTCTTGAGTTCTTGATGATTTCCAACAATGGCCTGGT | |
| CACTACCCTGTGGTTTATCTTCCTGCTTGTGTCCTACACAGTCATCCTAATGACGCTGAGGT | |
| CTCAGGCAGGAGGGGGCAGGAGGAAAGCCATCTCCACTTGCACCTCCCCACATCACTGTG | |
| GTGACCCTGCATTTTGTGCCCTGCATCTATGTCTATGCCCGGCCCTTCACTGCCCTCCCCAC | |
| AGAAAAGGCCATCTCTGTCACCTTCACTGTCATCTCCCCTCTGCTGAACCCTTTGATCTACA | |
| CTCTGAGGAACCAGGAAATGAAGTCAGCCATGAGAAGACTGAAGAGAAGACTCGTGCCTT | |
| CTGA | |
| AOLER151 sequences: | |
| MFSPNHTIVTEFILLGLTDDPVLEKILFGVFLAIYLITLAGNLCMILLIRTNSHLQTPMTTFLGHLS | (SEQ ID NO:279) |
| FVDICYSSNVTPNMLHNFLSEQKTISYAGCFTQCLLFIALVITEFYILASMALDRYVAICSPLHYS | |
| SRMSKNICVCLVTIPYMYGFLSGFSQSLLTFHLSFCGSLEINHFYCADPPLIMLACSDTRVKKMA | |
| MFVVAGFNLSSSLFIILLSYLFIFAAIFRIRSAEGRHKAFSTCASHLTIVTLFYGTLFCMYVRPPSE | |
| KSVEESKITAVFYTFLSPMLNPLIYSLRNTDVILAMQQMIRGKSFHKIAV | |
| ATGTTCTCCGCAAACCACACCATAGTGACAGAATTCATTCTCTTGGGACTGACAGACGACC | (SEQ ID NO:280) |
| CAGTGCTAGAGAAGATCCTGTTTGGGGTATTCCTTGCGATCTACCTAATCACACTGGCAGG | |
| CAACCTGTGCATGATCCTGCTGATCAGGACCAATTCCCACCTGCAAACACCCATGTATTTC | |
| TTCCTTGGCCACCTCTCCTTTGTAGACATTTGCTATTCTTCCAATGTTACTCCAAATATGCT | |
| GCACAATTTCCTCTCAGAACAGAAGACCATCTCCTACGCTGGATGCTTCACACAGTGTCTT | |
| CTCTTCATCGCCCTGGTGATCACTGAGTTTTACATCCTTGCTTCAATGGCATTGGATCGCTA | |
| TGTAGCCATTTGCAGCCCTTTGCATTACAGTTCCAGGATGTCCAAGAACATCTGTGTCTGT | |
| CTGGTCACTATCCCTTACATGTATGGGTTTCTTAGTGGGTTCTCTCAGTCACTGGTAACCTT | |
| TCACTTATCCTTCTGTGGCTCCCTTGAAATCAATCATTTCTACTGCGCTGATCCTCCTCTTA | |
| TCATGCTGGCCTGCTCTGACACCCGTGTCAAAAAGATGGCAATGTTTGTAGTTGCAGGCTT | |
| TAATCTCTCAAGCTCTCTCTTCATCATTCTTCTGTCCTATCTTTTCATTTTTGCAGCGATCTT | |
| CAGGATCCGTTCTGCTGAAGGCAGGCACAAAGCCTTTTCTACGTGTGCTTCCCACCTGACA | |
| ATAGTCACTTTGTTTTATGGAACCCTCTTCTGCATGTACGTAAGGCCTCCATCAGAGAAGT | |
| CTGTAGAGGAGTCCAAAATAACTGCAGTCTTTTATACTTTTTTGAGCCCAATGCTGAACCC | |
| ATTGATCTATAGCCTACGGAACACAGATGTAATCCTTGCCATGCAACAAATGATTAGGGGA | |
| AAATCCTTTCATAAAATTGCAGTTTAG | |
| AOLFR152 sequences: | |
| MDQINHTNVKEFFFLELTRSRELEFFLFVVFFAVYVATVLGNALIVVTITCESRLHTPMTTLLRN | (SEQ ID NO:281) |
| KSVLDIVFSSITVPKFLVDLLSDRKTISYNDCMAQIFFFHFAGGADIFFLSVMAYDRYLAIAKPL | |
| HYVTMMRKEVWVALVVASWVSGGLHSIIQVILMLPFPFCGPNTLDAFYCYVLQVVKLACTDT | |
| FALELFMISNNGLVTLLWFLLLLGSYTVILVMLRSHSGEGRNKALSTCTSHMLVVTLHFVPCV | |
| YIYCRPFMTLPMDTTISINNTVITPMLNPIIYSLRNQEMKSAMQRLQRRLGPSESRKWG | |
| ATGGACCAGATCAACCACACTAATGTGAAGGAGTTTTTCTTCCTGGAACTTACACGTTCCC | (SEQ ID NO:282) |
| GAGAGCTGGAGTTTTTCTTGTTTGTGGTCTTCTTTGCTGTGTATGTAGCAACAGTCCTGGG | |
| AAATGCACTCATTGTGGTCACTATTACCTGTGAGTCCCGCCTACACACTCCTATGTACTTTC | |
| TCCTGCGGAACAAATCAGTCCTGGACATCGTTTTTTCATCTATCACCGTCCCCAAGTTCCTG | |
| GTGGATCTTTTATCAGACAGGAAAACCATCTCCTACAATGACTGCATGGCACAGATCTTTT | |
| TCTTCCACTTTGCTGGTGGGGCAGATATTTTTTTCCTCTCTGTGATGGCCTATGACAGATAC | |
| CTTGCAATCGCCAAGCCCCTGCACTATGTGACCATGATGAGGAAAGAGGTGTGGGTGGCC | |
| TTGGTGGTGGCTTCTTGGGTGAGTGGTGGTTTGCATTCAATCATCCAGGTAATTCTGATGC | |
| TTCCATTCCCCTTCTGTGGCCCCAACACACTGGATGCCTTCTACTGTTATGTGCTCCAGGTG | |
| GTAAAACTGGCCTGCACTGACACCTTTGCTTTGGAGCTTTTCATGATCTCTAACAACGGAC | |
| TGGTGACCCTGCTCTGGTTCCTCCTGCTCCTGGGCTCCTACACTGTCATTCTGGTGATGCTG | |
| AGATCCCACTCTGGGGAGGGGCGGAACAAGGCCCTCTCCACGTGCACGTCCCACATGCTG | |
| GTGGTGACTCTTCACTTCGTGCCTTGTGTTTACATCTACTGCCGGCCCTTCATGACGCTGCC | |
| CATGGACACAACCATATCCATTAATAACACGGTCATTACCCCCATGCTGAACCCCATCATC | |
| TATTCCCTGAGAAATCAAGAGATGAAGTCAGCCATGCAGAGGCTGCAGAGGAGACTTGGG | |
| CCTTCCGAGAGCAGAAAATGGGGGTGA | |
| AOLFR153 sequences: | |
| MSKTSLVTAFILTGLPHAPGLDAPLFGIFLVVYVLTVLGNLLILLVIRVDSHLHTPMYTTLTNLS | (SEQ ID NO:283) |
| FIDMWFSTVTVPKMLMTLVSPSGRAISFHSCVAQLTTFHFLGSTECFLYTVMSYDRYLAISYPL | |
| RYTSMMSGSRCALLATSTWLSGSLHSAVQTILTFHLPYCGPNQIQHYLCDAPPILKLACADTSA | |
| NEMVIFVDIGLVASGCFLLIVLSYVSIVCSILRIHTSEGRHRAFQTCASHCIVVLCFFVXCVFIYLR | |
| PGSRDVVDGVVAIFYTVLTPLLNPVVYTLRNKEVKKAVLKLRDKVAHSQGE | |
| ATGTCCAAGACCAGCCTCGTGACAGCGTTCATCCTCACGGGCCTTCCCCATGCCCCAGGGC | (SEQ ID NO:284) |
| TGGACGCCCCACTCTTTGGAATCTTCCTGGTGGTTTACGTGCTCACTGTGCTGGGGAACCT | |
| CCTCATCCTGCTGGTGATCAGGGTGGATTCTCACCTCCACACCCCCATGTACTACTTCCTCA | |
| CCAACCTGTCCTTCATTGACATGTGGTTCTCCACTGTCACGGTGCCCAAAATGCTGATGAC | |
| CTTGGTGTCCCCAAGCGGCAGGGCTATCTCCTTCCACAGCTGCGTGGCTCAGCTCTATTTTT | |
| TCCACTTCCTGGGGAGCACCGAGTGTTTCCTCTACACAGTCATGTCCTATGATCGCTACTTG | |
| GCCATCAGTTACCCGCTCAGGTACACCAGCATGATGAGTGGGAGCAGATGTGCCCTCCTGG | |
| CCACCAGCACTTGGCTCAGTGGCTCTCTGCACTCTGCTGTCCAGACCATATTGACTTTCCAT | |
| TTGCCCTACTGTGGACCCAACCAGATCCAGCACTATTTGTGTGATGCACCGCCCATCCTGA | |
| AACTGGCCTGTGCAGACACCTCAGCCAACGAGATGGTCATCTTTGTGGACATTGGGCTAGT | |
| GGCCTCGGGCTGCTTTCTCCTGATAGTGCTGTCTTATGTGTCCATCGTCTGTTCCATCCTGC | |
| GGATCCACACCTCAGAGGGGAGGCACAGAGCCTTTCAGACCTGTGCCTCCCACTGCATCGT | |
| GGTCCTTTGCTTTTTTGTNNCCTGTGTTTTCATTTACCTGAGACCAGGCTCCAGGGACGTCG | |
| TGGATGGAGTTGTGGCCATTTTCTACACTGTGCTGACACCCCTTCTCAACCCTGTTGTGTAC | |
| ACCCTGAGAAACAAGGAGGTGAAGAAAGCTGTGTTGAAACTGAGAGACAAAGTAGCACAT | |
| TCTCAGGGAGAATAA | |
| AOLFR156 sequences: | |
| MCWAMPSPFTGSSTRNMESRNQSTVTEFIFTGFPQLQDGSLLTTFPLLFIYTFIIIDNLLIFSAVRL | (SEQ ID NO:285) |
| DTHLGNPMYNFISIFSFLEIWYTTATIPKMLSNLISEKKAISMTGCILQMTTFHSLENSEGILLTT | |
| MAIDRYVAICNPLRYQMIMTPRLCAHLSAGSCLFGFLILLPEIVMISTLPFCGPNQIHQIFCDLVP | |
| VLSLACTDTSMILIEDVIHAVTIIITFLIIALSYVRIVTVILRIPSSEGRQKAXSTCAGHLMVFLIFFG | |
| SVSLMYLRFSNTYPPVLDTAIALMFTVLAPFFNPIIYSLPIIYSLRNKDMNNAIKKLFCLQKVLNKPGG | |
| ATGTGCTGGGCTATGCGCTCTCCATTTACAGGTAGCTCTACTAGAAATATGGAGAGCAGAA | (SEQ ID NO:286) |
| ACCAATCAACAGTGACTGAATTTATCTTCACTGGATTCCCTCAGCTTCAGGATGGTAGTCT | |
| CCTGTACTTCTTTCCTTTACTTTTCATCTATACTTTTATTATCATTGATAACTTATTAATCTT | |
| CTCTGCTGTAAGGCTGGACACCCATCTGGGCAACCCCATGTATAATTTTATCAGTATATTTT | |
| CCTTTCTGGAGATCTGGTACACCACAGCCACCATTCCCAAGATGCTCTCCAACCTCATCAG | |
| TGAAAAGAAGGCCATCTCAATGACTGGCTGCATCTTGCAGATGTATTTCTTCCACTCACTT | |
| GAAAACTCAGAGGGGATCTTGCTGACCACCATGGCCATTGACAGATACGTTGCCATCTGCA | |
| ACCCTCTTCGCTATCAAATGATCATGACCCCCCGGCTCTGTGCTCACCTCTCTGCAGGTTCC | |
| TGCCTCTTCGGTTTCCTTATCCTGCTTCCCGAGATTGTGATGATTTCCACACTGCCTTTCTG | |
| TGGGCCCAACCAAATCCATCAGATCTTCTGTGACTTGGTCCCTGTGCTAAGCCTGGCCTGT | |
| ACAGACACGTCCATGATTCTGATTGAGGATGTGATTCATGCTGTGACCATCATCATTACCT | |
| TCCTAATCATTGCCCTGTCCTATGTAAGAATTGTCACTGTGATATTGAGGATTCCCTCTTCT | |
| GAAGGGAGGCAAAAGGCTNTTTCTACCTGTGCAGGCCACCTCATGGTCTTCCTGATATTCT | |
| TTGGCAGTGTATCACTCATGTACTTGCGTTTCAGCAACACTTATCCACCAGTTTTGGACAC | |
| AGCCATTGCACTGATGTTTACTGTACTTGCTCCATTCTTCAATCCCATCATTTATAGCCTGA | |
| GAAACAAGGACATGAACAATGCAATTAAAAAACTGTTCTGTCTTCAAAAAGTGTTGAACA | |
| AGCCTGGAGGTTAA | |
| AOLFR157 sequences: | |
| MAMDNVTAVFQFLLIGISNYPQWRDTFFTLVLIIYLSTLLGNGFMIFLIHFDPNLHTPITTFLSNL | (SEQ ID NO:287) |
| SFLDLCYGTASMPQALVHCFSTHPYLSYPRCLAQTSVSLALATAECLLLAAMAYDRVVAISNP | |
| LRYSVVMNGPVCVCLVATSWGTSLVLTAMLILSLRLHFCGANVINHFACEILSLIKLTCSDTSL | |
| NEFMILITSIFTLLLPFGFVLLSYIRIAMAMAIIRIRSLQGRLKAFTTCGSHLTVVTIFYGSAISMYMKT | |
| QSKSSPDQDKFISVFYGALTPMLNPLIYSLRKKDVKRAIRKVMLKRT | |
| ATGGCCATGGACAATGTCACAGCAGTGTTTCAGTTTCTCCTTATTGGCATTTCTAACTATCC | (SEQ ID NO:288) |
| TCAATGGAGAGACACGTTTTTCACATTAGTGCTGATAATTTACCTCAGCACATTGTTGGGG | |
| AATGGATTTATGATCTTTCTTATTCACTTTGACCCCAACCTCCACACTCCAATCTACTTCTT | |
| CCTTAGTAACCTGTCTTTCTTAGACCTTTGTTATGGAACAGCTTCCATGCCCCAGGCTTTGG | |
| TGCATTGTTTCTCTACCCATCCCTACCTCTCTTATCCCCGATGTTTGGCTCAAACGAGTGTC | |
| TCCTTGGCTTTGGCCACAGCAGAGTGCCTCCTACTGGCTGCCATGGCCTATGACCGTGTGG | |
| TTGCTATCAGCAATCCCCTGCGTTATTCAGTGGTTATGAATGGCCCAGTGTGTGTCTGCTT | |
| GGTTGCTACCTCATGGGGGACATCACTTGTGCTCACTGCCATGCTCATCCTATCCCTGAGG | |
| CTTCACTTCTGTGGGGCTAATGTCATCAACCATTTTGCCTGTGAGATTCTCTCCCTCATTAA | |
| GCTGACCTGTTCTGATACCAGCCTCAATGAATTTATGATCCTCATCACCAGTATCTTCACCC | |
| TGCTGCTACCATTTGGGTTTGTTCTCCTCTCCTACATACGAATTGCTATGGCTATCATAAGG | |
| ATTCGCTCACTCCAGGGCAGGCTCAAGGCCTTTACCACATGTGGCTCTCACCTGACCGTGG | |
| TGACAATCTTCTATGGGTCAGCCATCTCCATGTATATGAAAACTCAGTCCAAGTCCTCCCC | |
| TGACCAGGACAAGTTTATCTCAGTGTTTTATGGAGCTTTGACACCCATGTTGAACCCCCTG | |
| ATATATAGCCTGAGAAAAAAAGATGTTAAACGGGCAATAAGGAAAGTTATGTTGAAAAGG | |
| ACATGA | |
| AOLFR158 sequences: | |
| MKAGNFSDTPEFFLLGLSGDPELPILFMLFLSMYLATMLGNLLIILAVNSDSHLHTPMTTFLSI | (SEQ ID NO:289) |
| LSLVDICFTSTTMPKMLVNIQAQAQSINYTGCLTQICFVLVFVGLENGILMMAYDRFVAICHP | |
| LRYNVIMNPKLCGLLLLLSFIVSVLDALLHTLMVLQLTFCIDLEIPHFFCELAHILKACSDVLIN | |
| NILVYLVTSLLGVVPLSGIIFSYTRIVSSVMKIPSAGGKYKAFSICGSHLIVVSLFYGTGFGVYLSS | |
| GATHSSRKGAIASVMYTVVTPMLNPLIYSLRNKDMLKALRKLISRIPSFH | |
| ATGAAAGCAGGAAACTTCTCAGACACTCCAGAATTCTTTCTCTTGGGATTGTCAGGGGATC | (SEQ ID NO:290) |
| CGGAGCTGCAGCCCATCCTCTTCATGCTGTTCCTGTCCATGTACCTGGCCACAATGCTGGG | |
| GAACCTGCTCATCATCCTGGCCGTCAACTCTGACTCCCACCTCCACACCCCCATGTACTTCC | |
| TCCTCTCTATCCTGTCCTTGGTCGACATCTGTTTCACCTCCACCACGATGCCCAAGATGCTG | |
| GTGAACATCCAGGCACAGGCTCAATCCATCAATTACACAGGCTGCCTCACCCAAATCTGCT | |
| TTGTCCTGGTTTTTGTTGGATTGGAAAATGGAATTCTGGTCATGATGGCCTATGATCGATT | |
| TGTGGCCATCTGTCACCCACTGAGGTACAATGTCATCATGAACCCCAAACTCTGTGGGCTG | |
| CTGCTTCTGCTGTCCTTCATCGTTAGTGTCCTGGATGCTCTGCTGCACACGTTGATGGTGCT | |
| ACAGCTGACCTTCTGCATAGACCTGGAAATTCCCCACTTTTTCTGTGAACTAGCTCATATTC | |
| TCAAGCTCGCCTGTTCTGATGTCCTCATCAATAACATCCTGGTGTATTTGGTGACCAGCCT | |
| GTTAGGTGTTGTTCCTCTCTCTGGGATCATTTTCTCTTACACACGAAGTCTCCTCTGTCA | |
| TGAAAATTCCATCAGCTGGTGGAAAGTATAAAGCTTTTTCCATCTGCGGGTCACATTTAAT | |
| CGTTGTTTCCTTGTTTTATGGAACAGGGTTTGGGGTGTACCTTAGTTCTGGGGCTACCCACT | |
| CCTCCAGGAAGGGTGCAATAGCATCAGTGATGTATACCGTGGTCACCCCCATGCTGAACCC | |
| ACTCATTTACAGCCTGAGAAACAAGGACATGTTGAAGGCTTTGAGGAAACTAATATCTAG | |
| GATACCATCTTTCCATTGA | |
| AOLFR159 sequences: | |
| MGPRNQTAVSEFLLMKVTEDPELKLIPFSLFLSMYLVTILGNLLILLAVISDSHLHTPMTTLLFN | (SEQ ID NO:291) |
| LSFTDICLTTTTVPKILVNIQAQNQSITYTGCLTQICLVLVFAGLESCFLAVMAYDRYVAICHPL | |
| RYTVLMNVHFWGLLILLSMFMSTMDALVQSLMVLQLSFCKNVEIPLFFCEVVQVIKLACSDTL | |
| INNILITTASSVFGAIPLSGIIFSYSQIVTSVLRMPSARGKYKAFSTCGCHLSVFSLFYGTAFGVYIS | |
| SAVAESSRITAVASVMYTVVPQMMNPFIYSLRNKEMKKALRKLIGRLFPF | |
| ATGGGACCCAGAAACCAAACAGCTGTTTCAGAATTTCTTCTCATGAAAAGTGACAGAGGAC | (SEQ ID NO:292) |
| CCAGAACTGAAGTTAATCCCTTTCAGCCTGTTCCTGTCCATGTACCTGGTCACCATCCTGG | |
| GGAACCTGCTCATTCTCCTGGCTGTCATCTCTGACTCCCACCTCCACACCCCCATGTACTTC | |
| CTTCTCTTTAATCTCTCCTTTACTGACATCTGTTTAACCACAACCACAGTCCCAAAGATCCT | |
| AGTGAACATCCAAGCTCAGAATCAGAGTATCACTTACACAGGCTGCCTCACCCAGATCTGT | |
| CTTGTCTTGGTTTTTGCTGGCTTGGAAAGTTGCTTTCTTGCAGTCATGGCCTACGACCGCTA | |
| TGTGGCCATTTGCCACCCACTGAGGTACACAGTCCTCATGAATGTCCATTTCTGGGGCTTG | |
| CTGATTCTTCTCTCCATGTTCATGAGCACTATGGATGCCCTGGTTCAGAGTCTGATGGTATT | |
| GCAGCTGTCCTTCTGCAAAAACGTTGAAATCCCTTTGTTCTTCTGTGAAGTCGTTCAGGTC | |
| ATCAAGCTCGCCTGTTCTGACACCCTCATCAACAACATCCTCATATATTTTGCAAGTAGTGT | |
| ATTTGGTGCAATTCCTCTCTCTGGAATAATTTTCTCTTATTCTCAAATAGTCACCTCTGTTC | |
| TGAGAATGCCATCAGCAAGAGGAAAGTATAAAGCGTTTTCCACCTGTGGCTGTCACCTCTC | |
| TGTTTTTTCCTTGTTCTATGGGACAGCTTTTGGGGTGTACATTAGTTCTGCTGTTGCTGAGT | |
| CTTCCCGAATTACTGCTGTGGCTTCAGTGATGTACACTGTGGTCCCTCAAATGATGAACCC | |
| CTTCATCTACAGCCTGAGAAATAAGGAGATGAAGAAAGCTTTGAGGAAACTTATTGGTAG | |
| GCTGTTTCCTTTTTAG | |
| AOLFR160 sequences: | |
| MPMQLLLTDFIIFSIRFIINSMEARNQTAISKFLLLGLIEDPELQPVLFSLFLSMYLVTILGNLLILL | (SEQ ID NO:293) |
| AVISDSHLHTPMTTFLSNLSFLDICLSTTTIPKMLVNIQAQNRSITYSGCLTQICFVLFFAGLENC | |
| LLAAMAYDRYVAICHPLRYTVIMNPRLCGLLILLSLLTSVVNALLLSLMVLRLSFCTDLEIPLFF | |
| CELAQVIQLTCSDTLINNILITTAACIFGGVPLSGIILSYTQITSCVLRMPSASGKHKAVSTCGSHL | |
| SIVLLFYGAGLGVYISSVVTDSPRKTAVASVMYSVFPQMVNPFIYSLRNKDMKGTLRKFIGRIP | |
| SLLWCAICFGFRFLE | |
| ATGCCGATGCAGCTGCTGCTTACAGATTTTATTATCTTTTCCATCAGATTCATCATCAACAG | (SEQ ID NO:294) |
| CATGGAAGCGAGAAACCAAACAGCTATTTCAAAATTCCTTCTCCTGGGACTGATAGAGGAT | |
| CCGGAACTGCAGCCCGTCCTTTTCAGCCTGTTCCTGTCCATGTACTTGGTCACCATCCTGGG | |
| GAACCTGCTCATCCTCTTGGCTGTCATCTCTGACTCTCACCTCCACACCCCCATGTACTTCT | |
| TCCTCTCCAATCTCTCCTTTTTGGACATTTGTTTAAGCACAACCACGATCCCAAAGATGCTG | |
| GTGAACATGCAAGCTCAGAATCGGAGCATCACGTACTCAGGCTGCCTCACCCAGATCTGCT | |
| TTGTCTTGTTTTTTGCTGGCTTGGAAAATTGTCTCCTTGCAGCAATGGCCTATGACCGCTAT | |
| GTGGCCATTTGTCACCCCCTTAGATACACAGTCATCATGAACCCCCGCCTCTGTGGCCTGC | |
| TGATTCTTCTCTCTCTGTTGACTAGTGTTGTGAATGCCCTTCTTCTCAGCCTGATGGTGTTG | |
| AGGCTGTCCTTCTGCACAGACCTGGAAATCCCGCTCTTCTTCTGTGAACTGGCTCAGGTCA | |
| TCCAACTCACCTGTTCAGACACCCTCATCAATAACATCCTGATATATTTTGCAGCTTGCATA | |
| TTTGGTGGTGTTCCTCTGTCTGGAATCATTTTGTCTTACACTCAGATCACCTCCTGTGTTTT | |
| GAGAATGCCATCAGCAAGTGGAAAGCACAAAGCAGTTTCCACCTGTGGGTCTCACCTCTCC | |
| ATTGTTCTCTTGTTCTATGGGGCAGGTTTGGGGGTGTACATTAGTTCTGTGGTTACTGACTC | |
| ACCTAGGAAGACTGCAGTGGCTTCAGTGATGTATTCTGTGTTCCCTCAAATGGTGAACCCC | |
| TTTATCTATAGTCTGAGGAATAAGGACATGAAAGGAACCTTGAGGAAGTTCATAGGGAGG | |
| ATACCTTCTCTTCTGTGGTGTGCCATTTGCTTTGGATTCAGGTTTCTAGAGTAA | |
| AOLFR161 sequences: | |
| MEPRNQTSASQFILLGLSEKPEQETLLFSLFFCMYLVMVVGNLLIILAISIDSHLHTPMTTFLANL | (SEQ ID NO:295) |
| SLVDFCLATNTIPKMLVSLQTGSKAISYPCCLIQMTTFHFFGIVDSVIIAMMAYDRFVAICHPLH | |
| YAKIMSLRLCRLLVGALWAFSCFISLTHILLMARLVFCGSHEVPHTTCDLTPILRLSCTDTSVNR | |
| IFILIVAGMVIATPFVCILASYARILVAIMKVPSAGGRKKAFSTCSSHLSVVALFYGTTIGVYLCP | |
| SSVLTTVKEKASAVMYTAVTPMLNPFIYSLRNRDLKGALRKLVNRKITSSS | |
| ATGGAACCAAGAAACCAAACCAGTGCATCTCAATTCATCCTGCTGGGACTCTCAGAAAAGC | (SEQ ID NO:296) |
| CAGAGCAGGAGACGCTTCTCTTTTCCCTGTTCTTCTGCATGTACCTGGTCATGGTCGTGGG | |
| GAACCTGCTCATCATCCTGGCCATCAGCATAGACTCCCACCTCCACACCCCCATGTACTTCT | |
| TCCTGGCCAACCTGTCCCTGGTTGATTTCTGTCTGGCCACCAACACCATCCCTAAGATGCT | |
| GGTGAGCCTTCAAACCGGGAGCAAGGCCATCTCTTATCCCTGCTGCCTGATCCAGATGTAC | |
| TTCTTCCATTTCTTTGGCATCGTGGACAGCGTCATAATCGCCATGATGGCTTATGACCGGTT | |
| CGTGGCCATCTGCCACCCATTGCACTACGCCAAGATCATGAGCCTACGCCTCTGTCGCCTG | |
| CTGGTCGGCGCCCTCTGGGCGTTTTCCTGCTTCATCTCACTCACTCACATCCTCCTGATGGC | |
| CCGTCTCGTTTTCTGCGGCAGCCATGAGGTGCCTCACTACTTCTGCGACCTCACTCCCATCC | |
| TCCGACTTTCGTGCACGGACACCTCTGTGAATAGGATCTTCATCCTCATTGTGGCAGGGAT | |
| GGTGATAGCCACGCCCTTTGTCTGCATCCTGGCCTCCTATGCTCGCATCCTTGTGGCCATCA | |
| TGAAGGTCCCCTCTGCAGGCGGCAGGAAGAAAGCCTTCTCCACCTGCAGCTCCCACCTGTC | |
| TGTGGTTGCTCTCTTCTATGGGACCACCATTGGCGTCTATCTGTGTCCCTCCTCGGTCCTCA | |
| CCACTGTGAAGGAGAAAGCTTCTGCGGTGATGTACACAGCAGTCACCCCCATGCTGAATCC | |
| CTTCATCTACAGCTTGAGGAACAGAGACCTGAAAGGGGCTCTCAGGAAGCTGGTCAACAG | |
| AAAGATCACCTCATCTTCCTGA | |
| AOLFR162 sequences: | |
| MMRLMKEVRGRNQTEVTEFLLLGLSDNPDLQGVLFALFLLIYMANMVGNLGMIVLIKIDLCLH | (SEQ ID NO:297) |
| TPMTTFLSSLSFVDASYSSSVTPKMLVNLMAENKAISFHGCAAQFTTFGSFLGTECFLLAMMA | |
| YDRYAAIWNPLLYPVLVSGRICFLLIATSFLAGCGNAAIHTGMTFRLSFCGSNRINHFYCDTPPL | |
| LKLSCSDTHFNGIVIMAFSSFIVISCVMIVLISYLCIFIAVLKMPSLEGRHKAFSTCASYLMAVTIF | |
| FGTILFMYLRPTSSYSMEQDKVVSVFYTVIIPVLNPLIYSLKNKDVKKALKKILWKHIL | (SEQ ID NO:298) |
| ATGATGAGACTTATGAAAGAGGTTCGAGGCAGAAATCAAACAGAAGTAACAGAATTTCTC | |
| CTCTTAGGACTTTCCGACAATCCAGATCTACAAGGAGTCCTCTTTGCATTGTTTCTGTTGAT | |
| CTATATGGCAAACATGGTGGGCAATTTGGGGATGATTGTATTAAGATTAAGATTGATCTCTGT | |
| CTCCACACCCCCATGTATTTCTTTCTCAGTAGCCTCTCTTTTGTAGATGCCTCTTACTCTTCT | |
| TCCGTCACTCCCAAGATGCTGGTGAACCTCATGGCTGAGAATAAGGCCATTTCTTTTCATG | |
| GATGTGCTGCCCAGTTCTACTTCTTTGGCTCCTTCCTGGGGACTGAGTGCTTCCTGTTGGCC | |
| ATGATGGCATATGACCGCTATGCAGCCATTTGGAACCCCCTGCTCTACCCAGTTCTCGTGT | |
| CTGGGAGAATTTGCTTTTTGCTAATAGCTACCTCCTTCTTAGCAGGTTGTGGAAATGCAGC | |
| CATACATACAGGGATGACTTTTAGGTTGTCCTTTTGTGGTTCTAATAGGATCAACCATTTCT | |
| ACTGTGACACCCCGCCACTGCTCAAACTCTCTTGCTCTGATACCCACTTCAATGGCATTGTG | |
| ATCATGGCATTCTCAAGTTTTATTGTCATCAGCTGTGTTATGATTGTCCTCATTTCCTACCT | |
| GTGTATCTTCATTGCCGTCTTGAAGATGCCTTCGTTAGAGGGCAGGCACAAAGCCTTCTCC | |
| ACCTGTGCCTCTTACCTCATGGCTGTCACCATATTCTTTGGAACAATCCTCTTCATGTACTT | |
| GCGCCCTACATCTAGCTACTCAATGGAGCAAGACAAGGTTGTCTCTGTCTTTTATACAGTA | |
| ATAATCCCTGTGCTAAATCCCCTCATCTATAGTTTAAAAAATAAGGATGTAAAAAAGGCCC | |
| TAAAGAAGATCTTATGGAAACACATCTTGTAG | |
| AOLFR163 sequences: | |
| MQRSNHTVTEFILLGFTTDPGMQLGLFVVFLGVYSLTVVGNSTLIVLICNDSCLHTPMTTFTGN | (SEQ ID NO:299) |
| LSFLDLWYSSVYTPKILVTCISEDKSISFAGCLCQFFFSAGLAYSECYLLAAVAYDRYVAISKPL | |
| LYAQAMSIKLCALLVAVSYCGGFINSSIITKKTFSFNFCRENIIDDFFCDLLPLVELACGEKGGYK | |
| IMMTTLLASNVICPAVLILASYLFIITSVLRISSSKGYLKAFSTCSSHLTSVTLYYGSILYIYALPRS | |
| SYSFDMDKIVSTFYTVVFPMLNLMIYSLRNKDVKEALKKLLP | |
| ATGCAGAGGAGCAATCATACAGTGACTGAGTTTATACTGCTGGGCTTCACCACAGACCCA | (SEQ ID NO:300) |
| GGAATGCAGCTGGGCCTCTTCGTGGTGTTCCTGGGCGTGTACTCTCTCACTGTGGTAGGAA | |
| ATAGCACCCTCATCGTGTTGATCTGTAATGACTCCTGCCTCCACACACCCATGTATTTTTTC | |
| ACTGGAAATCTGTCGTTTCTGGATCTCTGGTATTCTTCTGTCTACACCCCAAAGATCCTAGT | |
| GACCTGCATCTCTGAAGACAAAAGCATCTCCTTTGCTGGCTGCCTGTGTCAGTTCTTCTTCT | |
| CTGCAGGGCTGGCCTATAGTGAGTGCTACCTGCTGGCTGCCGTGGCTTATGACCGCTACGT | |
| GGCCATCTCCAAGCCCCTGCTTTATGCCCAGGCCATGTCCATAAAGCTGTGTGCATTGCTG | |
| GTAGCAGTCTCATATTGTGGTGGCTTTATTAACTCTTCAATCATCACCAAGAAAACGTTTTC | |
| CTTTAACTTCTGCCGTGAAAACATCATTGATGACTTTTTCTGTGATTTGCTTCCCTTGGTGG | |
| AGCTGGCCTGTGGCGAGAAGGGCGGCTATAAAATTATGATGTACTTCCTGCTGGCCTCCAA | |
| TGTCATCTGCCCCGCAGTGCTCATCCTGGCCTCCTACCTCTTTATCATCACCAGTGTCTTGA | |
| GGATCTCCTCCTCCAAGGGCTACCTCAAAGCCTTCTCCACATGCTCCTCCCACCTGACCTCT | |
| GTCACTTTATACTATGGCTCCATTCTCTACATCTACGCTCTCCCCAGATCTAGCTATTCTTT | |
| TGATATGGACAAAATAGTTTCTACATTTTACACTGTGGTATTCCCCATGTTGAATCTCATG | |
| ATCTACAGCCTAAGGAATAAGGATGTGAAAGAGGCTCTGAAAAAACTTCTCCCATAA | |
| AOLFR164 sequences: | |
| MFLTERNTTSEATFTLLGFSDYLELQIPLFFVFLAVYGFSVVGNLGMIVIIKINPKLHTPMTTFLN | (SEQ ID NO:301) |
| HLSFVDFCYSSIIALPMMLVNLVVEDRTISFSGCLVQFFFFCTFVVTELILFAVMAYDIIFVAICNP | |
| LLYTVAISQKLCAMLVVVLYAWGVACSLTLACSALKLSFHGFNTINHFFCELSSLISLSYPDSYL | |
| SQLLLFTVATFNEISTLLIILTSYAFIIVTTLKMPSASGHRKVFSTCASHLTAITIFHGTILFLYCVP | |
| NSKNSRHTVKVASVFYTVVIPLLNPLIYSLRNKDVKDAIRKIINTKTTHIKHRHWYPFNFVIEQ | |
| ATGTTTCTGACAGAGAGAAATACGACATCTGAGGCCACATTCACTCTCTTGGGCTTCTCAG | (SEQ ID NO:302) |
| ATTACCTGGAACTGCAAATTCCCCTCTTCTTTGTATTTCTGGCAGTCTACGGCTTCAGTGTG | |
| GTAGGGAATCTTGGGATGATAGTGATCATCAAAATTAACCCAAAATTGCATACCCCCATGT | |
| ATTTTTTCCTCAACCACCTCTCCTTTGTGGATTTCTGCTATTCCTCCATCATTGCTCCCATGA | |
| TGCTGGTGAACCTGGTTGTAGAAGATAGAACCATTTCATTCTCAGGATGTTTGGTGCAATT | |
| CTTTTTCTTTTGCACCTTTGTAGTGACTGAATTAATTCTATTTGCGGTGATGGCCTATGACC | |
| ACTTTGTGGCCATTTGCAATCCTCTGCTCTACACAGTTGCCATCTCCCAGAAACTCTGTGCC | |
| ATGCTGGTGGTTGTATTGTATGCATGGGGAGTCGCATGTTCCCTGACACTCGCGTGCTCTG | |
| CTTTAAAGTTATCTTTTCATGGTTTCAACACAATCAATCATTTCTTCTGTGAGTTATCCTCC | |
| CTGATATCACTCTCTTACCCTGACTCTTATCTCAGCCAGTTGCTTCTTTTCACTGTTGCCAC | |
| TTTTAATGAGATAAGCACACTACTCATCATTCTGACATCTTATGCATTCATCATTGTCACCA | |
| CCTTGAAGATGCCTTCAGCCAGTGGGCACCGCAAAGTCTTCTCCACCTGTGCCTCCCACCT | |
| GACTGCCATCACCATCTTCCATGGCACCATCCTCTTCCTCTACTGTGTACCCAACTCCAAAA | |
| ACTCCAGGCACACAGTCAAAGTGGCCTCTGTGTTTTACACCGTGGTGATCCCCTTGTTGAA | |
| TCCCCTGATCTACAGTCTGAGAAATAAAGATGTTAAGGATGCAATCCGAAAAATAATCAAT | |
| ACAAAATATTTTCATATTAAACATAGGCATTGGTATCCATTTAATTTTGTTATTGAACAATA | |
| A | |
| AOLFR165 sequences: | |
| MAVGRNNTIVTKFILLGLSDHPQMKIFLFMLFLGLYLLTLAWNLSLIALIKMDSHLHMPMTTFL | (SEQ ID NO:303) |
| SNLSFLDICYVSSTAPKMLSDIITEQKTISFVGCATQTTVFGGMGLTECFLLAAMAYDRYAAICN | |
| PLLYTVLISHTLCLKMVVGAYVGGFLSSFIETYSVYQHDFCGPYMINHFFCDLPPVLALSCSDTF | |
| TSEVVTFIVSVVVGIVSVLVVLISYGYIVAAVVKISSATGRTKAFSTCASHLTAVTLFYGSGFFM | |
| YMRPSSSYSLNRDKVVSIFYALVIPVVNPIIYSFRNKEIKNAMRKAMERDPGISHGGPFIFMTLG | |
| ATGGCTGTAGGAAGGAACAACACAATTGTGACAAAATTCATTCTCCTGGGACTTTCAGACC | (SEQ ID NO:304) |
| ATCCTCAAATGAAGATTTTCCTTTTCATGTTATTTCTGGGGCTCTACCTCCTGACGTTGGCC | |
| TGGAACTTAAGCCTCATTGCCCTCATTAAGATGGACTCTCACCTGCACATGCCCATGTACT | |
| TCTTCCTCAGTAACCTGTCCTTCCTGGACATCTGCTATGTGTCCTCCACCGCCCCTAAGATG | |
| CTGTCTGACATCATCACAGAGCAGAAAACCATTTCCTTTGTTGGCTGTGCCACTCAGTACT | |
| TTGTCTTCTGTGGGATGGGGCTGACTGAATGCTTTCTCCTGGCAGCTATGGCCTATGACCG | |
| GTATGCTGCAATCTGCAACCCCTTGCTTTACACAGTCCTCATATCCCATACACTTTGTTTAA | |
| AGATGGTGGTTGGCGCCTATGTGGGTGGATTCCTTAGTTCTTTCATTGAAACATACTCTGT | |
| CTATCAGCATGATTTCTGTGGGCCCTATATGATCAACCACTTTTTCTGTGACCTCCCTCCAG | |
| TCCTGGCTCTGTCCTGCTCTGATACCTTCACCAGCGAGGTGGTGACCTTCATAGTCAGTGTT | |
| GTCGTTGGAATAGTGTCTGTGCTAGTGGTCCTCATCTCTTATGGTTACATTGTTGCTGCTGT | |
| TGTGAAGATCAGCTCAGCTACAGCTAGGACAAAGGCCTTCAGCACTTGTGCCTCTCACCTG | |
| ACTGCTGTGACCCTCTTCTATGGTTCTGGATTCTTCATGTACATGCGACCCAGTTCCAGCTA | |
| CTCCCTAAACAGGGACAAGGTGGTGTCCATATTCTATGCCTTGGTGATCCCCGTGGTGAAT | |
| CCCATCATCTACAGTTTTAGGAATAAGGAGATTAAAAATGCCATGAGGAAAGCCATGGAA | |
| AGGGACCCCGGGATTTCTCACGGTGGACCATTCATTTTTATGAGCCTTGGGCTAA | |
| AOLFR166 sequences: | |
| MEMENCTRVKEFIFLGLTQNREVSLVLFLFLLLVYVTTLLGNLLIMVTVTCESRLHTPMTTLLH | (SEQ ID NO:305) |
| NLSIADICFSSITVPKVLVDLLSERKTISFNHCFTQMFLFHLIGGVDVFSLSVMALDRYVAISKPL | |
| HYATIMSRDHCIGLTVAAWLGGFVHSIVQISLLLPLPFCGPNVLDTFYCDVHRVLKLAHTDIFIL | |
| ELLMISNNGLLTTLWFFLLLVSYIVILSLPKSQAGEGRRKAISTCTSHITVVTLHFVPCIYVYARP | |
| FTALPMDKAISVTFTVISPLLNIPLIYTLRNHEMKSAMRRLKRRLVPSDRK | |
| ATGGAGATGGAAAACTGCACCAGGGTAAAAGAATTTATTTTCCTTGGCCTGACCCAGAATC | *SEQ ID NO:306) |
| GGGAAGTGAGCTTAGTCTTATTTCTTTTCCTACTCTTGGTGTATGTGACAACTTTGCTGGGA | |
| AACCTCCTCATCATGGTCACTGTTACCTGTGAATCTCGCCTTCACACGCCCATGTATTTTTT | |
| GCTCCATAATTTATCTATTGCCGATATCTGCTTCTCTTCCATCACAGTGCCCAAGGTTCTGG | |
| TGGACCTTCTGTCTGAAAGAAAGACCATCTCCTTCAATCATTGCTTCACTCAGATGTTTCTA | |
| TTCCACCTTATTGGAGGGGTGGATGTATTTTCTCTTTCGGTGATGGCATTGGATCGATATG | |
| TGGCCATCTCCAAGCCCCTGCACTATGCGACTATCATGAGTAGAGACCATTGCATTGGGCT | |
| CACAGTGGCTGCCTGGTTGGGGGGCTTTGTCCACTCCATCGTGCAGATTTCCCTGTTGCTC | |
| CCACTCCCTTTCTGCGGACCCAATGTTCTTGACACTTTCTACTGTGATGTCCACCGGGTCCT | |
| CAAACTGGCCCATACAGACATTTTCATACTTGAACTACTAATGATTTCCAACAATGGACTG | |
| CTCACCACACTGTGGTTTTTCCTGCTCCTGGTGTCCTACATAGTCATATTATCATTACCCAA | |
| GTCTCAGGCAGGAGAGGGCAGGAGGAAAGCCATCTCCACCTGCACCTCCCACATCACTGT | |
| GGTGACCCTGCATTTCGTGCCCTGCATCTATGTCTATGCCCGGCCCTTCACTGCCCTCCCCA | |
| TGGATAAGGCCATCTCTGTCACCTTCACTGTCATCTCCCCTCTGCTCAACCCCTTGATCTAC | |
| ACTCTGAGGAACCATGAGATGAAGTCAGCCATGAGGAGACTGAAGAGAAGACTTGTGCCT | |
| TCTGATAGAAAATAG | |
| AOLFR167 sequences: | |
| MSITKAWNSSSVTMFILLGFTDHPELQALLFVTFLGIYLTTLAWNLALIFLIRGDTHLHTPMTTF | (SEQ ID NO:307) |
| LSNLSFIDICYSSAVAPNMLTDFFWEQKTISFVGCAAQFFFFVGMGLSECLLLTAMAYDRYAAI | |
| SSPLLYPTIMTQGLCTRMVVGAYVGGFLSSLIQASSIFRLHFCGPNIINHFFCDLPPVLALSCSDT | |
| FLSQVVNFLVVVTVGGTSFLQLLISYGYIVSAVLKIPSAEGRWKACNTCASHLMVVTLLFGTAL | |
| FVYLRPSSSYLLGRDKVVSVFYSLVIPMLNPLIYSLRNKEIKDALWKVLERKKVFS | |
| ATGTCCATAACCAAAGCCTGGAACAGCTCATCAGTGACCATGTTCATCCTCCTGGGATTCA | (SEQ ID NO:308) |
| CAGACCATCCAGAACTCCAGGCCCTCCTCTTTGTGACCTTCCTGGGCATCTATCTTACCACC | |
| CTGGCCTGGAACCTGGCCCTCATTTTTCTGATCAGAGGTGACACCCATCTGCAGACACCCA | |
| TGTACTTCTTCCTAAGCAACTTATCTTTCATTGACATCTGCTACTCTTCTGCTGTGGCTCCC | |
| AATATGCTCACTGACTTCTTCTGGGAGCAGAAGACCATATCATTTGTGGGCTGTGCTGCTC | |
| AGTTTTTTTTCTTTGTCGGCATGGGTCTGTCTGAGTGCCTCCTCCTGACTGCTATGGCATAC | |
| GACCGATATGCAGCCATCTCCAGCCCCCTTCTCTACCCCACTATCATGACCCAGGGCCTCT | |
| GTACACGCATGGTGGTTGGGGCATATGTTGGTGGCTTCCTGAGCTCCCTGATCCAGGCCAG | |
| CTCCATATTTAGGCTTCACTTTTGCGGACCCAACATCATCAACCACTTCTTCTGCGACCTCC | |
| CACCAGTCCTGGCTCTGTCTTGCTCTGACACCTTCCTCAGTCAAGTGGTGAATTTCCTCGTG | |
| GTGGTCACTGTCGGAGGAACATCGTTCCTCCAACTCCTTATCTCCTATGGTTACATAGTGT | |
| CTGCGGTCCTGAAGATCCCTTCAGCAGAGGGCCGATGGAAAGCCTGCAACACGTGTGCCT | |
| CGCATCTGATGGTGGTGACTCTGCTGTTTGGGACAGCCCTTTTCGTGTACTTGCGACCCAG | |
| CTCCAGCTACTTGCTAGGCAGGGACAAGGTGGTGTCTGTTTTCTATTCATTGGTGATCCCC | |
| ATGCTGAACCCTCTCATTTACAGTTTGAGGAACAAAGAGATCAAGGATGCCCTGTGGAAG | |
| GTGTTGGAAAGGAAGAAAGTGTTTTCTTAG | |
| AOLFR168 sequences: | |
| MEKINNVTEFIFWGLSQSPEIEKVCFVVFSFFYIIILLGNLLIMLTVCLSNLFKSPMTTFLSFLSFV | (SEQ ID NO:309) |
| DICYSSVTAPKMIVDLLAKDKTISYVGCMLQLLGVHFFGCTEIFILTVMAYDRYVAICKPLHYM | |
| TIMNRETCNKMLLGTWVGGFLHSIIQVALVVQLPFCGPNEIDHTTCDVHPVLKLACTETYIVG | |
| VVVTANSGTIALGSFVILLISYSIIILVSLRKQSAEGRRKALSTCGSHIAMVVIFFGPCTFMYMRPD | |
| TTFSEDKMVAVFYTIITPMLNPLIYTLRNAEVKNAMKKLWGRNVFLEAKGK | |
| ATGGAAAAAATAAACAACGTAACTGAATTCATTTTCTGGGGTCTTTCTCAGAGCCCAGAGA | (SEQ ID NO:310) |
| TTGAGAAAGTTTGTTTTGTGGTGTTTTCTTTCTTCTACATAATCATTCTTCTGGGAAATCTC | |
| CTCATCATGCTGACAGTTTGCCTGAGCAACCTGTTTAAGTCACCCATGTATTTCTTTCTCAG | |
| CTTCTTGTCTTTTGTGGACATTTGTTACTCTTCAGTCACAGCTCCCAAGATGATTGTTGACC | |
| TGTTAGCAAAGGACAAAACCATCTCCTTATGTGGGGTGCATGTTGCAACTGCTTGGAGTAC | |
| ATTTCTTTGGTTGCACTGAGATCTTCATCCTTACTGTAATGGCCTATGATCGTTATGTGGCT | |
| ATCTGTAAACCCCTACATTATATGACCATCATGAACCGGGAGACATGCAATAAAATGTTAT | |
| TAGGGACGTGGGTAGGTGGGTTCTTACACTCCATTATCCAAGTGGCTCTGGTAGTCCAACT | |
| ACCCTTTTGTGGACCCAATGAGATAGATCACTACTTTTGTGATGTTCACCCTGTGTTGAAA | |
| CTTGCCTGCACAGAAACATACATTGTTGGTGTTGTTGTGACAGCCAACAGTGGTACCATTG | |
| CTCTGGGGAGTTTTGTTATCTTGCTAATCTCCTACAGCATCATCCTAGTTTCCCTGAGAAAG | |
| CAGTCAGCAGAAGGCAGGCGCAAAGCCCTCTCCACCTGTGGCTCCCACATTGCCATGGTCG | |
| TTATCTTTTTCGGCCCCTGTACTTTTATGTACATGCGCCCTGATACGACCTTTTCAGAGGAT | |
| AAGATGGTGGCTGTATTTTACACCATTATCACTCCCATGTTAAATCCTCTGATTTATACACT | |
| GAGAAATGCAGAAGTAAAGAATGCAATGAAGAAACTGTGGGGCAGAAATGTTTTCTTGGA | |
| GGCTAAAGGGAAATAG | |
| AOLFR169 sequences: | |
| MMDNHSSATEFHLLGFPGSQGLHHILFAIFFFFYLVTLMGNTVIIVIVCVDKRLQSPMTTFLSHL | (SEQ ID NO:311) |
| STLEILVTTIIVPMMLWGLLFLGCRQYLSLHVSLNFSCGTMEFALLGVMAVDRYVAVCNPLRY | |
| NIIMNSSTCIWVVIVSWVFGFLSEIWPIYATFQFTFRKSNSLDHFYCDRGQLLKISCDNTLLTEFI | |
| LFLMAVFILIGSLIPTIVSYTYIISTILKIPSASGRRKAFSTFASHFTCVVIGYGSCLFLYVKPKQTQ | |
| GVEYNKIVSLLVSVLTPFLNPFIFTLRNDKVKEALRDGMKRCCQLLKD | |
| ATGATGGACAACCACTCTAGTGCCACTGAATTCCACCTTCTAGGCTTCCCTGGGTCCCAAG | (SEQ ID NO:312) |
| GACTACACCACATTCTTTTTGCTATATTCTTTTTCTTCTATTTAGTGACATTAATGGGAAAC | |
| ACGGTCATCATTGTGATTGTCTGTGTGGATAAACGTCTGCAGTCCCCCATGTATTTCTTCCT | |
| CAGCCACCTCTCTACCCTGGAGATCCTGGTCACAACCATAATTGTCCCCATGATGCTTTGG | |
| GGATTGCTCTTCCTGGGATGCAGACAGTATCTTTCTCTACATGTATCGCTCAACTTTTCCTG | |
| TGGGACCATGGAGTTTGCATTACTTGGAGTGATGGCTGTGGACCGTTATGTGGCTGTGTGT | |
| AACCCTTTGAGGTACAACATCATTATGAACAGCAGTACCTGTATTTGGGTGGTAATAGTGT | |
| CATGGGTGTTTGGATTTCTTTCTGAAATCTGGCCCATCTATGCCACATTTCAGTTTACCTTC | |
| CGCAAATCAAATTCATTAGACCATTTTTACTGTGACCGAGGGCAATTGCTCAAACTGTCCT | |
| GCGATAACACTCTTCTCACAGAGTTTATCCTTTTCTTAATGGCTGTTTTTATTCTCATTGGT | |
| TCTTTGATCCCTACGATTGTCTCCTACACCTACATTATCTCCACCATCCTCAAGATCCCGTC | |
| AGCCTCTGGCCGGAGGAAAGCCTTCTCCACTTTTGCCTCCCACTTCACCTGTGTTGTGATTG | |
| GCTATGGCAGCTGCTTGTTTCTCTACGTGAAACCCAAGCAAACACAGGGAGTTGAGTACAA | |
| TAAGATAGTTTCCCTGTTGGTTTCTGTGTTAACCCCCTTCCTGAATCCTTTCATCTTTACTCT | |
| TCGGAATGACAAAGTCAAAGAGGCCCTCCGAGATGGGATGAAACGCTGCTGTCAACTCCT | |
| GAAAGATTAG | |
| AOLFR170 sequences: | |
| MSFTSLIPSLCFSLTLPFLFCYLSLLPFLSAFLFITRWLLAFLSLFSVSVPVSSVSSSMVLCLYLSVS | (SEQ ID NO:313) |
| ASPSVFCFSCMQGPILWIMANLSQPSEFVLLGFSSFGELQALLYGPFLMLYLLAFMGNTIIVMVI | |
| ADTHLHTPMTTFLGNFSLLEILVTMTAVPRMLSDLLVPHKVITFTGCMVQFTTHFSLGSTSFLIL | |
| TDMALDRFVAICHPLRYGTLMSRAMCVQLAGAAWAAPFLAMVPTVLSRAHLDYCHGDVINH | |
| FFCDNEPLLQLSCSDTRLLEFWDFLMALTFVLSSFLVTLISYGYIVTTVLRIPSASSCQKAFSTCG | |
| SHLTLVFIGYSSTIFLYVRPGKAHSVQVRKVVALVTSVLTPFLNPFILTFCNQTVKTVLQGQMQ | |
| RLKGLCKAQ | |
| ATGTCTTTCACTTCTCTCATACCCTCACTCTGTTTCTCCTTGACTCTCCCATTCCTGTTTTGT | (SEQ ID NO:314) |
| TATCTTTCTTTATTGCCGTTTCTTTCTGCTTTTCTGTTTATCACTCGCTGGCTACTTGCCTTT | |
| CTCTCTCTATTCTCTGTCTCTGTCCCTGTTTCTTCTGTTTCAAGTTCAATGGTTCTCTGTCTC | |
| TATCTCTCTGTTTCTGCCTCTCCGTCTGTCTTTTGTTTCTCTTGCATGCAGGGCCCCATACTG | |
| TGGATCATGGCAAATCTGAGCCAGCCCTCCGAATTTGTCCTCTTGGGCTTCTCCTCCTTTGG | |
| TGAGCTGCAGGCCCTTCTGTATGGCCCCTTCCTCATGCTTTATCTTCTCGCCTTCATGGGAA | |
| ACACCATCATCATAGTTATGGTCATAGCTGACACCCACCTACATACACCCATGTACTTCTTC | |
| CTGGGCAATTTTTCCCTGCTGGAGATCTTGGTAACCATGACTGCAGTGCCCAGGATGCTCT | |
| CAGACCTGTTGGTCCCCCACAAAGTCATTACCTTCACTGGCTGCATGGTCCAGTTCTACTTC | |
| CACTTTTCCCTGGGGTCCACCTCCTTCCTCATCCTGACAGACATGGCCCTTGATCGCTTTGT | |
| GGCCATCTGCCACCCACTGCGCTATGGCACTCTGATGAGCCGGGCTATGTGTGTCCAGCTG | |
| GCTGGGGCTGCCTGGGCAGCTCCTTTCCTAGCCATGGTACCCACTGTCCTCTCCCGAGCTC | |
| ATCTTGATTACTGCCATGGCGACGTCATCAACCACTTCTTCTGTGACAATGAACCTCTCCTG | |
| CAGTTGTCATGCTCTGACACTCGCCTGTTGGAATTCTGGGACTTTCTGATGGCCTTGACCTT | |
| TGTCCTCAGCTCCTTCCTGGTGACCCTCATCTCCTATGGCTACATAGTGACCACTGTGCTGC | |
| GGATCCCCTCTGCCAGCAGCTGCCAGAAGGCTTTCTCCACTTGCGGGTCTCACCTCACACT | |
| GGTCTTCATCGGCTACAGTAGTACCATCTTTCTGTATGTCAGGCCTGGCAAAGCTCACTCT | |
| GTGCAAGTCAGGAAGGTCGTGGCCTTGGTGACTTCAGTTCTCACCCCCTTTCTCAATCCCT | |
| TTATCCTTACCTTCTGCAATCAGACAGTTAAAACAGTGCTACAGGGGCAGATGCAGAGGCT | |
| GAAAGGCCTTTGCAAGGCACAATGA | |
| AOLFR171 sequences: | |
| MVGNLLIWVTTIGSPSLGSLMTTFLAYLSLMDAIYSTAMSPKLMIDLLCDKIAISLSACMGQLFI | (SEQ ID NO:315) |
| EHLLGGAEVFLLVVMAYDRYVAISKPLHYLNIMNRLVCILLLVVAMIGGFVHSVVQIVFLYSLP | |
| ICGPNVIDHSVCDMYPLLELLCLDTTTIGLTVVANGGIICMVIFTFLLISCGVILNFLKTYSQEER | |
| HKALPTCISHIIVVALVFVPCIFMYVRPVSNFPFDKLMTVFYSIITLMLNPLIYSLRQSEMKNAM | |
| KNLWCEKLSIVRKRVSPTLNIFIPSSKATNRR | |
| ATGGTGGGAAACCTCCTCATTTGGGTGACTACTATTGGCAGCCCCTCCTTGGGCTCCCTAA | (SEQ ID NO:316) |
| TGTACTTCTTCCTTGCCTACTTGTCACTTATGGATGCCATATATTCCACTGCCATGTCACCC | |
| AAATTGATGATAGACTTACTCTGTGATAAAATCGCTATTTCCTTGTCAGCTTGCATGGGTC | |
| AGCTCTTCATAGAACACTTACTTGGTGGTGCAGAGGTCTTCCTTTTGGTGGTGATGGCCTA | |
| TGATCGCTATGTGGCTATCTCTAAGCCGCTGCACTATTTGAACATCATGAATCGACTGGTT | |
| TGCATCCTTCTGTTGGTGGTGGCCATGATTGGAGGTTTTGTGCACTCTGTGGTTCAAATTGT | |
| CTTTCTGTACAGTCTACCAATCTGTGGCCCCAATGTTATTGACCACTCTGTCTGTGACATGT | |
| ACCCATTGTTGGAACTGTTGTGCCTTGACACCTACTTTATAGGACTCACTGTGGTTGCCAA | |
| TGGTGGAATAATTTGTATGGTCATCTTTACCTTTCTGCTAATCTCCTGTGGAGTCATCCTAA | |
| ACTTCCTTAAAACTTACAGTCAGGAAGAGAGGCATAAAGCCCTGCCTACCTGCATCTCCCA | |
| CATCATTGTGGTTGCCCTCGTTTTTGTTCCCTGTATTTTTATGTATGTTAGACCCGTTTCCA | |
| ACTTTCCCTTTGATAAATTAATGACTGTGTTTTATTCAATTATCACACTCATGTTGAATCCT | |
| TTAATATACTCGTTGAGACAATCAGAGATGAAAAATGCTATGAAAAATCTCTGGTGTGAA | |
| AAGTTAAGTATAGTTAGAAAAAGAGTATCTCCCACACTGAACATATTTATTCCTAGTTCTA | |
| AGGCAACAAATAGGCGGTAA | |
| AOLFR172 sequences: | |
| MAETLQLNSTFLHPNFFILTGFPGLGSAQTWLTLVFGPIYLLALLGNGALPAVVWIDSTLHQPM | (SEQ ID NO:317) |
| FLLLAILAATDLGLATSIAPGLLAVLWLGPRSVPYAVCLVQMFFVHALTAMESGVLLAMACDR | |
| AAAIGRPLHYPVLVTKACVGYAALALALKAVAIVVPFPLLVAKFEHFQAKTIGHTYCAHMAV | |
| VELVVGNTQATNLYGLALSLAISGMDILGITGSYGLIAHAVLQLPTREAHAKAFGTCSSHICVIL | |
| AFYIPGLFSYLAHRFGHHTVPKPVHILLSNIYLLLPPALNPLIYGARTKQIRDRLLETFTFRKSPL | |
| ATGGCAGAAACTCTACAACTCAATTCCACCTTCCTACACCCAAACTTCTTCATACTGACTG | (SEQ ID NO:318) |
| GCTTTCCAGGGCTAGGAAGTGCCCAGACTTGGCTGACACTGGTCTTTGGGCCCATTTATCT | |
| GCTGGCCCTGCTGGGCAATGGAGCACTGCCGGCAGTGGTGTGGATAGACTCCACACTGCA | |
| CCAGCCCATGTTTCTACTGTTGGCCATCCTGGCAGCCACAGACCTGGGCTTAGCCACATCT | |
| ATAGCCCCAGGGTTGCTGGCTGTGCTGTGGCTTGGGCCCCGATCTGTGCCATATGCTGTGT | |
| GCCTGGTCCAGATGTTCTTTGTACATGCACTGACTGCCATGGAATCAGGTGTGCTTTTGGC | |
| CATGGCCTGTGATCGTGCTGCGGCAATAGGGCGTCCACTGCACTACCCTGTCCTGGTCACC | |
| AAAGCCTGTGTGGGTTATGCAGCCTTGGCCCTGGCACTGAAAGCTGTGGCTATTGTTGTAC | |
| CTTTCCCACTGCTGGTGGCAAAGTTTGAGCACTTCCAAGCCAAGACCATAGGCCATACCTA | |
| TTGTGCACACATGGCAGTGGTAGAACTGGTGGTGGGTAACACACAGGCCACCAACTTATA | |
| TGGTCTGGCACTTTCACTGGCCATCTCAGGTATGGATATTCTGGGTATCACTGGCTCCTAT | |
| GGACTCATTGCCCATGCTGTGCTGCAGCTACCTACCCGGGAGGCCCATGCCAAGGCCTTTG | |
| GTACATGTAGTTCTCACATCTGTGTCATTCTGGCCTTCTACATACCTGGTCTCTTCTCCTAC | |
| CTCGCACACCGCTTTGGTCATCACACTGTCCCAAAGCCTGTGCACATCCTTCTCTCCAACAT | |
| CTACTTGCTGCTGCCACCTGCCCTCAACCCCCTCATCTATGGGGCCCGCACCAAGCAGATC | |
| AGAGACCGACTCCTGGAAACCTTCACATTCAGAAAAAGCCCGTTGTAA | |
| AOLFR173 sequences: | |
| MSHTNVTIFHPAVFVLPGIPGLEAYHIWLSIPLCLIYITAVLGNSILIVVIVMERNLHVPMTTFLS | (SEQ ID NO:319) |
| MLAVMDILLSTTTVPKALAIFWLQAHNIAFDACVTQGFFVHMMFVGESAILLAMAFDRFVAIC | |
| APLRYTTVLTWPVVGRIALAVITRSFCIIFPVIFLLKRLPFCLTNIVPHSYCEHIGVARLACADITV | |
| NIWYGFSVPIVMVILDVILIAVSYSLILRAVFRLPSQDARHKALSTCGSHLCVILMFYVPSFFTLL | |
| THHFGRNIPQHVHILLANLYVAVPPMLNPIVYGVKTKQIREGVAHRFFDIKTWCCTSPLGS | |
| ATGAGTCACACCAATGTTACCATCTTCCATCCTGCAGTTTTTGTCCTTCCTGGCATCCCTGG | (SEQ ID NO:320) |
| GTTGGAGGCTTATCACATTTGGCTGTCAATACCTCTTTGCCTCATTTACATCACTGCAGTCC | |
| TGGGAAACAGCATCCTGATAGTGGTTATTGTCATGGAACGTAACCTTCATGTGCCCATGTA | |
| TTTCTTCCTCTCAATGCTGGCCGTCATGGACATCCTGCTGTCTACCACCACTGTGCCCAAGG | |
| CCCTAGCCATCTTTTGGCTTCAAGCACATAACATTGCTTTTGATGCCTGTGTCACCCAAGGC | |
| TTCTTTGTCCATATGATGTTTGTGGGGGAGTCAGCTATCCTGTTAGCCATGGCCTTTGATCG | |
| CTTTGTGGCCATTTGTGCCCCACTGAGATATACAACAGTGCTAACATGGCCTGTTGTGGGG | |
| AGGATTGCTCTGGCCGTCATCACCCGAAGCTTCTGCATCATCTTCCCAGTCATATTCTTGCT | |
| GAAGCGGCTGCCCTTCTGCCTAACCAACATTGTTCCTCACTCCTACTGTGAGCATATTGGA | |
| GTGGCTCGTTTAGCCTGTGCTGACATCACTGTTAACATTTGGTATGGCTTCTCAGTGCCCAT | |
| TGTCATGGTCATCTTGGATGTTATCCTCATCGCTGTGTCTTACTCACTGATCCTCCGAGCAG | |
| TGTTTCGTTTGCCCTCCCAGGATGCTGGGCACAAGGCCCTCAGCACTTGTGGCTCCCACCT | |
| CTGTGTCATCCTTATGTTTTATGTTCCATCCTTCTTTACCTTATTGACCCATCATTTTGGGCG | |
| TAATATTCCTCAACATGTCCATATCTTGCTGGCCAATCTTTATGTGGCAGTGCCACCAATGC | |
| TGAACCCCATTGTCTATGGTGTGAAGACTAAGCAGATACGTGAGGGTGTAGCCCACCGGTT | |
| CTTTGACATCAAGACTTGGTGCTGTACCTCCCCTCTGGGCTCATGA | |
| AOLFR175 sequences: | |
| MHFLSQNDLNINLIPHLCLHRHSVIAGAFTIHRHMKIFNSPSNSSTFTGFILLGFPCPREGQILLFV | (SEQ ID NO:321) |
| LFTVVYLLTLMGNGSIICAVHWDQRLHAPMYILLANFSFLEICYVTSTVPSMLANFLSDTKIISF | |
| SGCFLQFTTFFSLGSTECFFLAVMAFDRYLAICRPLRYPTIMTRRLCTNLVVNCWVLGFIWFLIPI | |
| VNISQMSFCGSRIIDHFLCDPAPLLTLTCKKGPVIELVFSVLSPLPVFMLFLFIVGSYALVVRAVL | |
| RVPSAAGRRKAFSTCGSHLAVVSLFYGSVLVMYGSPPSKNEAGKQKTVTLFYSVVTPLLNPVI | |
| YSLRNKDMRKALKKFWGT | |
| ATGCATTTTCTTTCCCAAAATGATTTAAATATAAATCTGATTCCCCATCTATGTTTGCACCG | (SEQ ID NO:322) |
| TCATTCAGTAATTGCTGGTGCTTTTACAATTCACAGGCACATGAAAATCTTCAACAGCCCC | |
| AGCAACTCCAGCACCTTCACTGGCTTCATCCTCCTGGGCTTCCCTTGCCCCAGGGAGGGGC | |
| AGATCCTCCTCTTTGTGCTCTTCACTGTTGTTTACCTCCTGACCCTCATGGGCAATGGTTCC | |
| ATCATCTGTGCTGTGCACTGGGATCAGAGACTCCACGCCCCCATGTACATCCTGCTCGCCA | |
| ACTTCTCCTTCTTGGAGATATGTTATGTCACCTCCACAGTCCCCAGCATGCTGGCCAACTTC | |
| CTCTCTGACACCAAGATCATCTCGTTCTCTGGCTGCTTCCTCCAGTTCTACTTTTTCTTCTCC | |
| TTGGGCTCTACAGAATGCTTTTTCCTGGCAGTTATGGCATTTGATCGATACCTTGCCATCTG | |
| TCGGCCTCTACGCTATCCAACCATTATGACCAGACGTCTCTGTACCAATCTTGTGGTCAATT | |
| GCTGGGTACTTGGTTTCATCTGGTTCTTGATTCCTATCGTCAACATCTCCCAAATGTCCTTC | |
| TGTGGATCTAGGATTATTGACCACTTCCTATGTGACCCAGCTCCTCTTCTAACTCTCACTTG | |
| CAAAAAAGGCCCTGTGATAGAGCTTGTCTTTTCTGTCTTAAGTCCTCTGCCTGTCTTTATGC | |
| TCTTTCTCTTCATTGTGGGGTCCTATGCTCTGGTCGTGAGAGCTGTGTTGAGGGTCCCTTCA | |
| GCAGCTGGGAGAAGAAAGGCTTTCTCCACCTGTGGGTCTCACCTGGCTGTGGTTTCACTGT | |
| TCTACGGCTCAGTACTGGTCATGTATGGGAGCCCACCATCTAAGAATGAAGCTGGAAAGC | |
| AGAAGACTGTGACTCTGTTTTATTCTGTTGTTACCCCACTGGTTAACCCTGTGATATATAGT | |
| CTTAGGAACAAAGATATGAGAAAAGCTCTGAAGAAATTTTGGGGAACATAA | |
| AOLFR176 sequences: | |
| MFFIIHSLVTSVFLTALGPQNRTMHFVTEFVLLGFHGQREMQSCFFSFILVLYLLTLLGNGAIVC | (SEQ ID NO:323) |
| AVKLDRRLHTPMYILLGNFAFLEIWYISSTVPNMLVNILSEIKTISFSGCFLQFTTFFSLGTTECFF | |
| LSVMAYDRYLAICRIPLHYPSIMTGKFCIILVCVCWVGGFLCYPVPTVLISQLPFCGPNIIDHLVCD | |
| PGPLFALACISAPSTELICYTFNSMIIFGPFLSILGSYTLVIRAVLCIPSGAGRTKAFSTCGSHLMV | |
| VSLFYGTLMVMYVSPTSGNPAGMQKIITLVYTAMTPFLNPLIYSLRNKDMKDALKRVLGLTVS | |
| QN | |
| ATGTTCTTTATTATTCATTCTTTGGTTACTTCTGTTTTTCTAACAGCTTTGGGACCCCAGAA | (SEQ ID NO:324) |
| CAGAACAATGCATTTTGTGACTGAGTTTGTCCTCCTGGGTFPTCCATGGTCAAAGGGAGATG | |
| CAGAGCTGCTTCTTCTCATTCATCCTGGTTCTCTATCTCCTGACACTGCTAGGGAATGGAGC | |
| TATTGTCTGTGCAGTGAAATTGGACAGGCGGCTCCACACACCCATGTACATCCTTCTGGGA | |
| AACTTTGCCTTTCTAGAGATCTGGTACATTTCCTCCACTGTCCCAAACATGCTAGTCAATAT | |
| CCTCTCTGAGATTAAAACCATCTCCTTCTCTGGTTGCTTCCTGCAATTCTATTTCTTTTTTTC | |
| ACTGGGTACAACAGAGTGTTTCTTTTTATCAGTTATGGCTTATGATCGGTACCTGGCCATC | |
| TGTCGTCCATTACACTACCCCTCCATCATGACTGGGAAGTTCTGTATAATTCTGGTCTGTGT | |
| ATGCTGGGTAGGCGGATTTCTCTGCTATCCAGTCCCTATTGTTCTTATCTCCCAACTTCCCT | |
| TCTGTGGGCCCAACATCATFITGACCACTTGGTGTGTGACCCAGGCCCATTGTTTGCACTGGC | |
| CTGCATCTCTGCTCCTTCCACTGAGCTTATCTGTTACACCTTCAACTCGATGATTATCTTTG | |
| GGCCCYICCTCTCCATCTTGGGATCTTACACTCTGGTCATCAGAGCTGTGCTTTGTATFITCCC | |
| TCTGGTGCTGGTCGAACTAAAGCTTTCTCCACATGTGGGTCCCACCTAATGGTGGTGTCTC | |
| TATTCTATGGAACCCTTATGGTGATGTATGTGAGCCCAACATCAGGGAACCCAGCAGGAAT | |
| GCAGAAGATCATCACTCTGGTATACACAGCAATGACTCCATTCTFAAATCCCCTTATCTAT | |
| AGTCTTCGAAACAAAGACATGAAAGATGCTCTAAAGAGAGTCCTGGGGTTAACAGTTAGC | |
| CAAAACTGA | |
| AOLFR177 sequences: | |
| MSFFFVDLRPMNRSATHTVTEFILLGFPGCWKIQIFLFSLFLVTYVLTLLGNGAIIYAVRCNPLLH | (SEQ ID NO:325) |
| TPMTTLLGNFAFLEIWYVSSTLPNMLVNILSKTKAISFSGCFLQFTTFFSLGTTECLFLAVMAYD | |
| RYLAICHPLQYPAIMTVRFCGKLVSFCWLIGFLGYPIPIFYISQLPFCGPNIIDHFLCDMDPLMAL | |
| SCAIPAPITECIFYTQSSLVLFFTSMYILRSYILLLTAVFQVPSAAGRRKAFSTCGSHLVVVSLFYG | |
| TVMVMYVSPTYGIPTLLQKILTLVYSVTTPLFNPLIYTLRNKDMKLALRNVLFGMRIRQNS | |
| ATGTCTTTTCTTCTTTGTAGACTTAAGACCCATGAACAGGTCAGCAACACACATGGTGACAG | (SEQ ID NO:326) |
| AGTTTATTCTCCTGGGATTCCCTGGTTGCTGGAAGATTCAGATTTTCCTCTTCTCATTGTTT | |
| TTGGTGATTTATGTCTTGACCTTGCTGGGAAATGGAGCCATCATCTATGCAGTGAGATGCA | |
| ACCCACTACTACACACCCCCATGTACTTTCTGCTGGGAAATTTTGCCTTCCTTGAGATCTGG | |
| TATGTGTCCTCCACTATTCCTAACATGCTAGTCAACATTCTCTCCAAGACCAAGGCCATCTC | |
| ATTTTCTGGGTGCTTCCTCCAGTTTCTATTTCTTCTTTTCACTGGGAACAACTGAATGTCTCT | |
| TTCTGGCAGTAATGGGTTATGATCGATACCTGGCCATCTGCCACCCACTGCAGTACCCTGC | |
| CATCATGACTGTAAGGTTCTGTGGTAAGCTGGTGTCTTTCTGTTGGCTTATTGGATfCCTTG | |
| GATACCCAATTCCCATTTTCTACATCTCCCAACTCCCCTTCTGTGGTCCTAATATCATTGAT | |
| CACTTCCTGTGTGACATGGACCCATTGATGGCTCTATCCTGTGCCCCAGCTCCCATAACTG | |
| AATGTATTTTCTATACTCAGAGCTCCCTTGTCCTCTTTTTCACTAGTATGTACATTCTTCGA | |
| TCCTATATCCTGTTACTAACAGCTGTTTTTCAGGTCCCTTCTGCAGCTGGTCGGAGAAAAG | |
| CCTTCTCTACCTGTGGTTCTCATTTGGTTGTGGTATCTCTTTTCTATGGGACAGTCATGGTA | |
| ATGTATGTAAGTCCTACATATGGGATCCCAACTTTATTGCAGAAGATCCTCACACTGGTAT | |
| ATTCAGTAACGACTCCTCTTTTTAATCCTCTGATCTATACTCTTCGTAATAAGGACATGAAA | |
| CTCGCTCTGAGAAATGTCCTGTTTGGAATGAGAATTCGTCAAAATTCGTGA | |
| AOLFR178 sequences: | |
| MVGANHSVVSEFVFLGLTNSWEIIRLLLLVFSSMFYMASMMGNSLILLTVTSDPHLHSPMTTLL | (SEQ ID NO:327) |
| ANLSFTDLGVSSVTSPKMIYDLFRKHEVISFGGCIAQIFFIHVIGGVEMVLLIAMAFDRYVAICKP | |
| LQYLTIIVISPRMCMFFLVAAWVTGLIHSVVQLVFVVNLPFCGPNVSDSFYCDLPRFIKLACTDSY | |
| RLEFMVTANSGFISLGSFFILIISYVVIILTVLKHSSAGLSKALSTLSAHVSVVVLFFGPLIFVYTW | |
| PSPSTHLDKFLAIFDAVLTPVLNPIIYTFRN | |
| ATGGTTGGGGCAAATCACTCCGTGGTGTCAGAGTTTGTGTTCCTGGGACTCACCAATTCCT | (SEQ ID NO:328) |
| GGGAGATCCGACTTCTCCTCCTTGTGTTCTCCTCCATGTTTTTACATGGCCAGTATGATGGGA | |
| AACTCTCTCATTTTGCTCACTGTGACTTCTGACCCTCACTTGCACTCCCCCATGTATTTTCT | |
| GTTAGCCAACCTCTCCTTCATTGACCTGGGTGTTTGCTCTGTCACTTCTCCCAAAATGATTT | |
| ATGACCTGTTCAGAAAGCACGAAGTCATCTCCTTTGGAGGCTGCATCGCTCAAATCTTCTT | |
| CATCCACGTCATTGGCGGTGTGGAGATGGTGCTGCTCATAGCCATGGCCTTTGACAGATAT | |
| GTGGCCATATGTAAGCCCCTCCAGTACCTGACCATTATGAGCCCAAGAATGTGCATGTTCT | |
| TCTTAGTGGCTGCCTGGGTGACCGGCCTTATCCACTCTGTAGTTCAATTGGTTTTTGTAGTA | |
| AACTTGCGCTTCTGTGGTGCTAATGTATCGGACAGCTTTTACTGTGACCTTCCTCGGTTCAT | |
| CAAACTTGCCTGCACAGACAGCTACCGACTGGAGTTCATGGTTACAGCCAACAGTGGATTC | |
| ATCTCTCTGGGCTCCTTCTTCATACTGATCATTTCCTATGTGGTCATCATTCTCACTGTTCT | |
| GAAACACTCTTCAGCTGGTTTATCCAAGGCTCTGTCCACCCTTTCAGCTCACGTCAGTGTG | |
| GTAGTTTITGTTCTTTGGTCCTYPGATTTTTGTCTATACGTGGCCATCTCCCTCCACACACCT | |
| GGATAAGTTTCTGGCCATCTTTGATGCAGTTCTCACTCCTGTTTTAAATCCTATCATCTACA | |
| CATTCAGGAATTGA | |
| AOLFR179 sequences: | |
| MNGMNHSVVSEFVFMGLTNSREIQLLLFVFSLLFTTASMMGNLVWFTVTMDAHLHSPMYFL | (SEQ ID NO:329) |
| ANLSIIDMAFCSITAPKMICDIFKKHKAISFRGCITQIFFSHALGGTEMVLLIAMAFDRYMAICKP | |
| LHYLTIMSPRMCLTTLATSSIIGLIHSLVQLVFVVDLPFCGPNIFDSFYCDLPRLLRLACTNTQEL | |
| EFMVTVNSGLISVGSFVLLVISYIFILFTVWKHSSGGLAKALSTLSAHVTVVILFFGPLMFFYTW | |
| PSPTSHLDKYLAIFDAFITPFLNPVIYTFRNKDMKVAMRRICSRLAHFTKIL | |
| ATGAATGGAATGAATCACTCTGTGGTATCAGAATTTGTATTCATGGGACTCACCAACTCAC | (SEQ ID NO:330) |
| GGGAGATTCAGCTTCTACTTTTTGTTTTCTCTTTGTTGTTCTACTTTGCGAGCATGATGGGA | |
| AACCTTGTCATTGTATTCACTGTAACCATGGATGCTCATCTGCACTCCCCCATGTATTTCCT | |
| CCTGGCTAACCTCTCAATCATTGATATGGCATTTTGCTCAATTACAGCCCCTAAGATGATTT | |
| GTGATATTTTCAAGAAGCACAAGGCCATCTCCTTTCGGGGATGTATTACTCAGATCTTCTT | |
| TAGCCATGCTCTTGGGGGCACTGAGATGGTGCTGCTCATAGCCATGGCCTTTGACAGATAC | |
| ATGGCCATATGTAAACCTCTCCACTACCTGACCATCATGAGCCCAAGAATGTGTCTATACT | |
| TTTTAGCCACTTCCTCTATCATTGGCCTTATCCACTCATTGGTCCAATTAGTTTTTGTGGTA | |
| GATTTACCTTTTFFGTGGTCCTAATATCTTTGACAGTTTTTACTGTGATCTCCCTCGGCTCCT | |
| CAGACTTGCCTGTACCAACACCCAAGAACTGGAGTTCATGGTCACTGTCAATAGTGGACTC | |
| ATTTCTGTGGGCTCCTTTGTCTTGCTGGTAATTTCCTACATCTTCATTCTGTTCACTGTTTG | |
| GAAACATTCTTCTGGTGGTCTAGCCAAGGCCCTCTCTACCCTGTCAGCTCATGTCAGTGTG | |
| GTCATCTTGTTCTTTGGGCCACTGATGTTTTTCTACACATGGCCTTCTCCCACATCACACCT | |
| GGATAAATATCTTGCTATTTTTGATGCATTTATTACTCCTTTTCTGAATCCAGTTATCTACA | |
| CATTCAGGAACAAAGACATGAAAGTGGCAATGAGGAGACTGTGCAGTCGTCTTGCGCATT | |
| TTACAAAGATTTTGTAA | |
| AOLFR180 sequences: | |
| MTNKMYATTKNLNTTSFLIVQCLQPTMAIFNNTTSSSSNFLLTAFPGLECAHVWISIPVCCLYTI | (SEQ ID NO:331) |
| ALLGNSMIFLVIITKRRLHKPMYTTLSMLAAVDLCLTTTLPTVLGVLWFHAREISFKACFIQMF | |
| FVHAFSLLESSVLVAMAFDRFVAICNPLNYATILTDRMVLVIGLVICIRPAVFLLPLLVATNTVSF | |
| HGGHELSHPFCYHPEVIKYTYSKPWISSFWGLFLQLYLNGTDVLFILFSYVLILRTVLGIVARKK | |
| QQKALSTCVCHICAVTIFYVPLISLSLAIHRLFHSTPRVLCSTLANIYLLLPPVLNPIIYSLKTKTIR | |
| QAMFQLLQSKGSWGFNVRGLRGRWD | |
| ATGACTAATAAAATGTATGCTATATATATAAAGAATCTTAATTATTTTCTTTCCTCATAGT | (SEQ ID NO:332) |
| TCAGTGTCTTCAACCAACCATGGCAATATGATAACACCACTTCGTCTCCTCTACTTCC | |
| TCCTCACTGCATTCCCTGGGCTGGAATGTGCTCATGTCTGGATCTCCATTCCAGTCTGCTGT | |
| CTCTACACCATTGCCCTCTTGGGAAACAGTATGATCTTTCTTGTCATCATTACTTGCGGA | |
| GACTCCACAAACCCATGTATTATTTCCTCTCCATGCTGGCAGCTGTTGATCTATGTCTGACC | |
| ATTTACGACCCTTCCCACTGTGCTTGGTGTTCTCTGGTTTCATGCCCGGGAGATCAGCTTTT | |
| AGCTTGCTTCATTCAAATGTTCTTTGTGCATGCTTTCTCCTTGCTGGAGTCCTCGGTGCTGG | |
| TAGCCATGGCCTTTGACCGCTTCGTGGCTATCTGTAACCCAGTGAACTATGCTACTATCCTC | |
| ACAGACAGGATGGTCCTGGTGATAGGGCTGGTCATCTGCATTAGACCAGCAGTTTCTTAC | |
| TTCCCCTTCTTGTAGCCATAAACACTGTGTCTTTTCATGGGGGTCACGAGCTTTCCCATCCA | |
| TTTTGCTACCACCCAGAAGTGATCAAATAGACATATTCCAAACCTTGGATCAGCAGTTTTT | |
| GGGGACTGTTTCTTCAGCTCTACCTGAATGGCACTGACGTATTGTTTTTCTTTCTCCTAT | |
| GTCCTGATCCTCCGTACTGTTCTGGGCATTGTGGCCCGAAAGAAGCAACAAAAAGCTCTCA | |
| GCACTTGTGTCTGTCACATCTGTGCAGTCACTATTTTCTATGTGCCACTGATCAGCCTGTCT | |
| TTGGCACACCGCCTCTTCCACTCCACCCCAAGGGTGCTCTGTAGCACTTTGGCCAATATTTA | |
| TCTGCTCTTACCACCTGTGCTGAACCCTATCATTTACAGCTTTGAAGACCAAGACAATCCGC | |
| CAGGCTATGTTCCAGCTGCTCCAATCCAAGGGTTCATGGGGTTTTAATGTGAGGGGTCTTA | |
| GGGGAAGATGGGATTGA | |
| AOLFR181 sequences: | |
| MSVLNNSEVKLFLLIGIPGLEHAHIWFSIPICLMYLLAIMGNCTILFIIKTEPSLHEPMYTTLAML | (SEQ ID NO:333) |
| AVSDMGLSLSSLPTMLRVFLFNAMGISPNACFAQEFFIHGFTVMESSVLLIMSLDRFLAIHNPLR | |
| YSSILTSNRVAIKMGLILAIRSILLVIPFPFTLRRLKYCQKNLLSHSYCLHQDTMKLACSDNKTNV | |
| IYGFFIALCTMLDLALIVLSYYLILKTILSIASLAERLKALNTCVSHICAVLTFYVIPIITLAAMHHF | |
| AKHKSPLVVILIADMFLLVPPLMNPIVYCVKTRQIWETLGKLLNVCGR | |
| ATGTCTGTTCTCAATAACTCCGAAGTCAAGCTTTTCCTTCTGATTGGGATCCCAGGACTGG | (SEQ ID NO:334) |
| AACATGCCCACATTTGGTTCTCCATCCCCATTTGCCTCATGTACCTGCTTGCCATCATGGGC | |
| AACTGCACGATTCTCTTTATTATAAAGACAGAGCCCTCGCTTCATGAGCCCATGTATTATTT | |
| CCTTGCCATGTTGGCTGTCTCTGACATGGGCCTGTCCCTCTCCTCCCTTCCTACCATGTTGA | |
| GGGTCTTCTTGTTCAATGCCATGGGAATTTCACCTAATGCCTGCTTTGCTCAAGAATTCTC | |
| ATTCATGGATCACTGTCATGGAATCCTCAGTACTTCTAATTATGTCTTTGGACCGCTTTCT | |
| TGCCATTCACAATCCCTTAAGATACAGTTCTATCCTCACTAGCAACAGGGTTGCTAAAATG | |
| GGACTTATTTTAGCCATTAGGAGCATTCTCTTAGTGATTCCATTTCCCTTCACCTTAAGGAG | |
| ATTAAAATATTGTCAAAAGAATCTTCTTTCTCACTCATACTGTCTTCATCAGGATACCATGA | |
| AGCTGGCCTGCTCTGACAACAAGACCAATGTCATCTATGGCTTCTTCATTGCTCTCTGTACT | |
| ATGCTGGACTTGGCACTGATTGTTTTGTCTTATGTGCTGATCTTGAAGACTATACTCAGCAT | |
| TGCATCTTTGGCAGAGAGGCTTAAGGCCCTAAATACCTGTGTCTCCCACATCTGTGCTGTG | |
| CTCACCTTCTATGTGCCCATCATCACCCTGGCTGCCATGCATCACTTTGCCAAGCACAAAA | |
| GCCCTCTTGTTGTGATCCTTATTGGAGATATGTTCTTGTTGGTGCCGCCCCTTATGAACCCC | |
| ATTGTGTACTGTGTAAAGACTCGACAAATCTGGGAGAAGATCTTGGGGAAGTTGCTTAAT | |
| GTATGTGGGAGATAA | |
| AOLER182 sequences: | |
| MTLGSLGNSSSSVSATFLLSGIPGLERMHIWISIPLCFMTLVSIPGNCTILFIIKTERSLIIEPMTLFL | (SEQ ID NO:335) |
| SMLALIDLGLSLCTLPTVLGIFWVGAREISHDACFAQLFFIHCFSFLESSVLLSMAFDRFVAICHP | |
| LHYVSILTNTVIGRIGLVSLGRSVALIFPLPFMLKRFPTCGSPVLSHSTCLHQEVIVIKLACADMK | |
| ANSITGMFVIVSTVGIDSLLILFSTALILRTVLSIASRAERFKALNTCVSHICAVLLFTTPMIGLSV | |
| IHRFGKQAPHLVQVVMGFMTLLFPPVMNITIVTSVKTKQIRDRVTHAFCT | |
| ATGACCCTGGGATCCCTGGGAAACAGCAGCAGCAGCGTTTCTGCTACCTTCCTGCTGAGTG | (SEQ ID NO:336) |
| GCATCCCTGGGCTGGAGCGCATGCACATCTGGATCTCCATCCCACTGTGCTTCATGTATCT | |
| GGTTTCCATCCCGGGCAACTGCACAATTCTTTTTATCATTAAAACAGAGCGCTCACTTCAT | |
| GAACCTATGTATCTCTTCCTGTCCATGCTGGCTCTGATTGACCTGGGTCTCTCCCTTTGCAC | |
| TCTCCCTACAGTCCTGGGCATCTTTTTGGGTTGGAGCACGAGAAATTAGCCATGATGCCTGC | |
| TTTGCTCAGCTCTTTTTCATTCACTGCTTCTCCTTCCTCGAGTCCTCTGTGCTACTGTCTATG | |
| GCCTTTGACCGCTTTGTGGCTATCTGCCACCCCTTGGACTATGTTTCCATCTCACCTCAC | |
| AGTCATTGGCAGGATTGGCGTGGTCTCTGTGGGTCGTAGTGTAGCACTCATTTTTCCATA | |
| CCTTTTATGCTCAAAAGATTCCCCTATTGTGGCTCCCCAGTCTCTCACATTCTTATTGTCT | |
| CCACCAAGAAGTGATGAATTGGCCTGTGCCGACATGAAGGCCTCAGCATCTACGGCAT | |
| GTTTGTCATCGTCTCTACAGTGGGTATAGACTCACTGCTCATCCTCTTCTCTTATGCTCTGA | |
| TCCTGCGCACCGTGGTGTCCATCGCCTCCAGGGCTGAGAGATTCAAGGCCCTTTCACCTG | |
| TGTTTCCCACATCTGTGCTGTGCTGCTCTTCTACACTCCCATGATTGGCCTCTCTGTCATCC | |
| ATCGCTTTGGAAAGCAGGCACCCCACCTGGTCCAGGTGGTCATGGGTTTTCATGTATCTTCT | |
| CTTTCCTCCTGTGATGAATCCCATTGTCTACAGTGTGAAGACCTACAGATCCGGGATCGA | |
| GTGACGCATGCCTTTTGTTACTAA | |
| AOLFR183 sequences: | |
| MTNLNASQANHRFILTGIPGTPDKNPWLAFPLGFLTTLTLLGNGTILAVIKVEPSLHEPTYYFL | (SEQ ID NO:337) |
| SILALTDVSLSMSTLPSMLSITWFNAPQIVFDACIMQMFFIIIVFGIVESGVLVSMAFDRFVAIRN | |
| PLHTVSILTHDVIRKTGISVLTRAVCVVFPVPFLIKCLPFCHSNVLSHSTCLHQNMMITACASTR | |
| INSLYGLIVVIFTLGLDVLLTLLSTVLTLKTVLGIVSRGERLKTLSTCLSHMSTVLLFTVPFMGA | |
| ASMIHRPWEHLSPVVHMVMADITLLLPPVLNPIVTSVKTKQI | |
| ATGACGAACTTGAATGCATCACAGGCCAACCACCGTAACTTCATTCTGACAGGTATCCCAG | (SEQ ID NO:338) |
| GAACGCCAGACAAGAACCCATGGTTGGCCTTTCCCCTGGGATTTCTCTACACACTCACACT | |
| CCTGGGAAATGGTACCATCCTAGCTGTCATCAAGGTGGAGCCAAGTCTCCATGAGCCCACG | |
| TATTACTTTCCTTTCTATCTTGGCTCTCACTGACGTTAGTCTCTCCATGTCCACCTTGCCCTCC | |
| ATGCTCAGCATCTACTGGTTTAATGCCCCTCAGATTGTTTTGATGCATGCATCATGCAGAT | |
| GTTCTTCATCCATGTATTTGGAATAGTAGAATCAGGAGTCCTAGTGTCCATGGCCTTTGAC | |
| AGATTTGTGGCGATCCGAAACCCATTACACTATGTTTCCATCCTGACTCACGATGTTATTCG | |
| AAAGACTGGAATATCTGTCCTCACCCGGGCAGTCTGTGTGGTATTCCCTGTGCCCTTCCTT | |
| ATAAAGTGCCTACCCTTCTGCCATTCCAATGTCTTGTCTCATTCATACTGTCTTCACGAAAA | |
| CATGATGCGGCTAGCTTGTGCCAGCACCCGCATCAACAGCCTCTACGGCCTCATCGTCGTC | |
| ATCTTCACACTGGGGCTCGATGTTCTCCTCACTCTACTGTCTTATGTACTCACCCTGAAGAC | |
| TGTGCTGGGCATTGTCTCCAGAGGTGAAAGGCTGAAAACCCTCAGCACATGCCTCTCTCAC | |
| ATGTCTACCGTGCTCCTCTTCTATGTTCCTTTTATGGGTGCTGCCTCCATGATCCACAGAT | |
| TTGGGAGCATTTATCACCAGTAGTGCACATGGTCATGGCTGATATATACCTACTGCTCCCG | |
| CCTGTGCTAAACCCCATTGTCTACAGTGTGAAGACCAAGCAAATTTGA | |
| AOLFR184 sequences: | |
| MSTLPTQIAPNSSTSMAPTFLLVGMPGLSGAIPSWWTLPLIAVTLLSALGNGTILWIIALQPALHR | (SEQ ID NO:339) |
| PMHFFLFLLSVSDIGLVTALMPTLLGIALAGAIITVPASACLLQMVFIHVFSVMESSVLLAMSID | |
| RALAICRPLHTPALLTNGVISTSLAISFRCLGLHLPLPFLLATMPTCLPQVLTHSTCLHPDVARL | |
| ACPEAWGAATSLFVVLSAMGLDPLLIFFSTGLIGKVLQGVESREDRWKAGQTCAAHLSAVLLF | |
| YIPMILLALINHPELPITQHTHTLLSTVHFLLPPLIKPILTSVKMKEIRKRILNRLQPRKVGGAQ | |
| ATGTCAACATTACCAACTCAGATAGCCCCCAATAGCAGCACTTCAATGGCCCCCACCTTCT | (SEQ ID NO:340) |
| TGCTGGTGGGCATGCCAGGCCTATCAGGTGCACCCTCCTGGTGGACATTGCCCCTCATTGC | |
| TGTCTACCTTCTCTCTGCACTGGGAAATGGCACCATCCTCTGGATCATTGCCCTGCAGCCC | |
| GCCCTGCACCGCCCAATGCACTTCTTCCTCTTCTTGCTTAGTGTGTCTGATATTGGATTGGT | |
| CACTGCCCTGATGCCCACACTGCTGGGCATCGCCCTTGCTGGTGCTCACACTGTCCCTGCC | |
| TCAGCCTGCCTTCTACAGATGGTTTTTATCCATGTCITTTCTGTCATGGAGTCCTCTGTCTT | |
| GCTCGCCATGTGCATTGATCGGGCACTGGCCATCTGCCGACCTCTCCACTACCCAGCGCTC | |
| CTCACCAATGGTGTAATTAGCAAAATCAGCCTGGCCATTTCTTTTCGATGCCTGGGTCTCC | |
| ATCTGCCCCTGCCATTCCTGCTGGCCTACATGCCCTACTGCCTCCCACAGGTCCTAACCCAT | |
| TCTTATTGCTTGCATCCAGATGTGGCTCGTTTGGCCTGCCCAGAAGCTTGGGGTGCAGCCT | |
| ACAGCCTATTTGTGGTTCTTTCAGCCATGGGTTTGGACCCCCTGCTTATTTTCTTCTCCTAT | |
| GGCCTGATTGGCAAGGTGTTGCAAGGTGTGGAGTCCAGAGAGGATCGCTGGTGGCTGGT | |
| CAAACCTGTGCTGCCCACCTCTCTGCAGTGCTCCTCTTCTATATCCCTATGATCCTCCTGGC | |
| ACTGATTAACCATCCTGAGCTGCCAATCACTCAGCATAGCCATACTCTTCTATCCTATGTCC | |
| ATTTCCTTCTTCCTCCATTGATAAACCCTATTCTCTATAGTGTCAAGATGAAGGAGATTAGA | |
| AAGAGAATACTCAACAGGTTGCAGCCCAGGAAGGTGGGTGGTGCTCAGTGA | |
| AOLFR185 sequences: | |
| MFYPILNDISTKNNSNIMSCCNILFIKTVEIWVTNQTQSPWTPIVPSKSLVTNTTCFDCTTLQR | (SEQ ID NO:341) |
| VDCVPSRDHTNQSMVLASGNSSSHPVSFILLGIPGLESFQLWIAFPFCATTAVAVVGNITLLHVIR | |
| IDHTLHEPMTLFLAMLATTDLVLSSSTQPKMLAIFWFHHEIQTHACLIQVFFIITSSTSGVL | |
| MAMALDCTVATCFPLRIHSSILTPSVVIKLGTIVMLRGLLWVSPFCFMVSRMPFCQHQAIPQSYC | |
| EHMAVLKLVCADTSISRGTGLFVAFSVAGFDMIVIGMSTVMILRAVLQLPSGEARLTSTRA | |
| SHICVILALTPALFSFLTTRFGHD\TPRVVHILFANLTLLIPPMLNPIITGTTTKQIGDRVIQGCCG | |
| NIP | |
| ATGTTCTACCCCATTTTGAATGACATAAGTACGAAAAACAACAGTAACATCATGTCATGT | (SEQ ID NO:342) |
| GTAACATATTATTTATTAAAACAGTTGAAATTATTCTAGTTTATAATCAAACCCAATCACC | |
| CTGGTATCCAATAGTCCCATCCAAAAGCCTTGTATATAATAATAACACTTGTTTTGATTGTT | |
| ATCATCTGCAGAGAGTAGATTGCGTTCCCAGCAGAGACCATATTAACCAGTCCATGGTGCT | |
| GGCTTCAGGGAACAGCTCTTCTCATCCTGTGTCCTTCATCCTGCTTGGAATCCCAGGCCTG | |
| GAGAGTTTCCAGTTGTGGATTGCCTTTCCGTTCTGTGCCACGTATGCTGTGGCTGTTGTTGG | |
| AAATATCACTCTCCTCCATGTAATCAGAATTGACCACACCCTGCATGAGGCCATGTACCTC | |
| TTTCTGGCCATGCTGGCCATCACTGACCTGGTCCTCTCCTCCTCCACTCAACCTAAGATGTT | |
| GGCCATATTCTGGTTTCATGCTCATGAGATTCAGTACCATGCCTGCCTCATCCAGGTGTTCT | |
| TCATCCATGCCTTTTCTTCTGTGGAGTCTGGGGTGCTCATGGCTATGGCCCTGGACTGCTAC | |
| GTGGCTACCTGCTTCCCACTCCGACACTCTAGCATCCTGACCCCATCGGTCGTGATCAAAC | |
| TGGGGACCATCGTGATGCTGAGAGGGCTGCTGTGGGTGAGCCCCTTCTGCTTCATGGTGTC | |
| TAGGATGCCCTTCTGCCAACACCAAGCCATTCCCCAGTCATACTGTGAGCACATGGCTGTG | |
| CTGAAGTTGGTGTGTGCTGATACAAGCATAAGTCGTGGGTATGGGCTCTTTGTGGCCTTCT | |
| CTGTGGCTGGCTTTGATATGATTGTCATTGGTATGTCATACGTGATGATTTTGAGAGCTGT | |
| GCTTCAGTTGCCGTCAGGTGAAGCCCGCCTCAAAGCTTTTAGCACACGTGCCTCCCATATC | |
| TGTGTCATCTTGGCTCTTTATATCCCAGCCCTTTTTTCTTTCCTCACCTACCGCTTTGGCCAT | |
| GATGTGCCCCGAGTTGTACACATCCTGTTTGCTAATCTCTATCTAGTGATACCTCCCATGCT | |
| CAACCCCATCATTTATGGAGTTAGAACCAAACAGATCGGGGACAGGGTTATCCAAGGATG | |
| TTGTGGAAACATCCCCTGA | |
| AOLFR186 sequences: | |
| MSNASLVTAFILTGLPHAPGLDALLFGIFLVVTVLTVLGNLLILLVIRVDSHLHTPMTTTLTNLS | (SEQ ID NO:343) |
| FIDMWFSTVTVIPKMLMTLVSPSGRAISFHSCVAQLTTFHFLGSTECFLTTVMSTDRTLMSYPL | |
| RYTSMMSGSRCALLATGTWLSGSLHSAVQTILTFHLPTCGPNQIQHTTCDAPPILKLACADTSA | |
| NVMVIFVDIGIVASGCFVLIVLSTVSIVCSILRIRTSDGRRRAFQTCASHCIVVLCFFVPCVVIYLR | |
| PGSMDAMDGVVAIFTTVLTPLLNPVVTTLRNKEVKXAVLKLRDKVAHPQRK | |
| ATGTCCAACGCCAGCCTCGTGACAGCATTCATCCTCACAGGCCTTCCCCATGCCCCAGGGC | (SEQ ID NO:344) |
| TGGACGCCCTCCTCTTTGGAATCTTCCTGGTGGTTTACGTGCTCACTGTGCTGGGGAACCT | |
| CCTCATCCTGCTGGTGATCAGGGTGGATTCTCACCTCCACACCCCCATGTACTACTTCCTCA | |
| CCAACCTGTCCTTCATTGACATGTGGTTCTCCACTGTCACGGTGCCCAAAATGCTGATGAC | |
| CTTGGTGTCCCCAAGCGGCAGGGCTATCTCCTTCCACAGCTGCGTGGCTCAGCTCTATTTTT | |
| TCCACTTCCTGGGGAGCACCGAGTGTTTGCTCTACACAGTCATGTCCTATGATCGCTACTTG | |
| GCCATCAGTTACCCGCTCAGGTACACCAGCATGATGAGTGGGAGCAGGTGTGCCCTCCTGG | |
| CCACCGGCACTTGGCTCAGTGGCTCTCTGCACTCTGCTGTCCAGACCATATTGACTTTCCAT | |
| TTGCCCTACTGTGGACCCAACCAGATCCAGCAGTACTTCTGTGACGCACGGCCCATCCTGA | |
| AACTGGCCTGTGCAGACACCTCAGCCAACGTGATGGTCATCTTTGTGGACATTGGGATAGT | |
| GGCCTCAGGCTGCTTTGTCCTGATAGTGCTGTCCTATGTGTCCATCGTCTGTTCCATCCTGC | |
| GGATCCGCACCTCAGATGGGAGGCGCAGAGCCTTTCAGACCTGTGCCTCCCACTGTATTGT | |
| GGTCCTTTGCTTCTTTGTTCCCTGTGTTGTCATTTATCTGAGGCCAGGCTCCATGGATGCCA | |
| TGGATGGAGTTGTGGCCATTTTCTACACTGTGCTGAGGCCCCTTCTCITCCCTGTTGTGTAC | |
| ACCCTGAGAAACAAGGAGGTGAAGAAAGCTGTGTTGAAACTTAGAGACTGTAGCACAT | |
| CCTCAGAGGAAATAA | |
| AOLFR187 sequences: | |
| MAQVRALHTMALFSANSIGAMNSDTRIAGCFLTGWGLEQLHTLSWFCITTLEGNGILI | (SEQ ID NO:345) |
| CVILSQAILHEPMTFLSMLASADVLLSTTTMPKALANLWLGTSHISFDGCLTQTFIHFLFIHSA | |
| VLLAEDRTVAICSPLRTVTILTSKVIGKWTATLSRSFIIMFPSIFLLEHLTCQTMTTFCEH | |
| MGIAHLSCSDISEVWTGLAAALLSTGLDIMLITVSTTLQALSQDITSTLSTCGSHICV | |
| ILLFYVPALFSVFAYRFGGRSIPCYVHILLASLYVVIPPMLNPVIYGVRTKPILEGAKQMFSNLAK | |
| GSK | |
| ATGGCACAGGTGAGGGCGCTGCATAAAATCATGGCCCTTTTTTCTGCTTCAGCATAGGTG | (SEQ ID NO:346) |
| CTATGAACAACTCTGACACTCGCATAGGAGGCTGCTTCCTCACTGGCATCCCTGGGCTGGA | |
| GCAACTACATATCTGGCTGTCCATCCCCTTCTGGATCATGTACATCGCTGCCCTGGTGGC | |
| AATGGCATCCTAATTTGTGTCATCCTCTCCCAGGCAATCCTGCATGAGCCCATGTACATAT | |
| TCTTATCTATGCTGGCCAGTGCTGATGTCTTGCTCTCTACCACCACCATGCCTTGGCCCTG | |
| GGCAATTTGTGGCTAGGTTATAGCGACATTTCCTTTGATGGCTGCCTCACTCTTGTTCTT | |
| CATTCACTTCCTCTTCATTCACTCTGCTGTGCTGGTGGCCATGGGCTTTGACCGCTATGTGG | |
| CCATCTGCTCCCCCCTGCGATATGTCACAATCCTCACAAGCAAGGTCATTGGGTTGATCGT | |
| CACTGCCACCCTGAGCCGCAGCTTCATCATTATGTTTCCATCCATCTTTCTCCTTGAGCACC | |
| TGCACTATTGCCAGATCAACATCATTGCACACACATTTTGTGAGCACATGGGCATGCCCA | |
| TCTGTCCTGTTCTGATATCTCCATCAATGTCTGGTATGGGTTGGCAGCTGCTCTTCTCTCCA | |
| CAGGCCTGGACATCATGCTTATTACTGTTTCCTACATCCACATCCTCCAATGCAGTCTCCGC | |
| CTCCTTTTCTCAAGATGCCCGCTCCAAGGCCCTGAGTACCTGTGGATCCCATATCTGTGTCAT | |
| CCTACTCTTCTATGTCCCTGCCCTTTTTTCTGTCTTTGGCTACAGGTTTGGTGGGAGTGCA | |
| TCCCATGCTATGTCCATATTCTCCTGGCCAGCCTCTACGTTGTCATCCTGCTATGCTCTT | |
| CCCGTTATTTATGGAGTGAGGACTAAGCCAATACTGGAAGGGGCTTGCAGATGTTTTCA | |
| AATCTTGCCAAAGGATGTAAATAA | |
| AOLFR188 sequences: | |
| MFPSLCPCVLLVQLPLMNENMQCFVFCSCDSLLRMMVSRFIHVPFVKMKMIVGGTSTFFSN | (SEQ ID NO:347) |
| ELLCVRPWSGKTWSIRHHIFDMELLTNNLKEITDPFVCRLTILSPTPSEEHMTTTEFILL | |
| GLTQNPEGQKVLFVTFLLITMVTIMGNLLIIVTIMASQSLGSPMTFLASLSFTTTSTAFAPK | |
| MIVDLLSEKKTISFQGCMAQLFMDHLFAGAEVILLVVMATDRTMAICKPLHELITMNRRVCVL | |
| MLLAAWIGGFLHSLVQFLFIYQLPFCGPNVIDNFLCDLYPLLKLACTNTYVTGLSMIANGGAIC | |
| AVTFFTILLSTGVILIISLKTQSLEGKRXAETTCASHVTVVILFFTCIFLTATNSTFPIDKSMTV | |
| VLTFITPMLNPLITTLKNAEMKSAMRKLWSTKVSLAGKWLYHS | |
| ATGTTCCCCTCCCTGTGTCCATGTGTTCTCCTTGTTCAACTCCCACTTATGAATGAGTCAT | (SEQ ID NO:348) |
| GCAGTGTTTTGTTTTCTGTTCTTGTGATAGTTTGCTGAGAATGATGGTTTCCCGCTTCATCC | |
| ATGTCCCATTTGTAAAAATGAAAAGGATAATTGTGGGAGGATATTCTTTACACTTCTTTTC | |
| TAATGAGCTGCTCTGTGTGAGGCCCTGGTCAGGGAAAACGTGGTCGATTGGCATCACAT | |
| TTTTGACATGGAGCTTGTGACAAATAATCTCAAATTTATCACTGACCCTTTTGTTTGTAGGC | |
| TCCGACACCTGAGTCCAACACCTTCAGAAGAACACATGAAAATTGITCTTGTGACTG | |
| AATTTATCCTCTTAGGGCTCACACAGAACCCTGAGGGGCAAAAGGTTTTATTGTCACAT | |
| CTTACTAATCTACATGGTGACGATAATGGGCAACCTGCTTTATCATAGTGAGCATCATGGCC | |
| AGCCAGTCCCTGGGTTCCCCCATGTACTTTTTTCTGGCTTCTTTATCATTCATAGATACCGT | |
| CTATTCTACTGCATTGCTCCCAAAATGATTGTTGACTTGCTCTCTGAGTGACCATT | |
| CCTTTCAGGGTTGTATGGCTCAACTTTTTATGGATCATTTATTTGCTGGTGCTGITGTCATT | |
| CTTCTGGTGGTAATGGCCTATGATCGATACATGGCCATCTGTAAGCCTCTTCATGTTTGA | |
| TCACCATGAATCGTCGAGTCTGTGTTCTTATGCTGTTGGCGGCCTGGATTGGAGGCTTTCT | |
| TCACTCATTGGTTCAATTTCTCTTTATTTATCAGCTCCCTTTCTGTGGACCCTTGTCATTG | |
| ACAACTCCTGTGTGATTTGTATCCCTTATTGAAACTTGCTTGCACCAATTACCTATGTCACT | |
| GGGCTTTCTATGATAGCTAATGGAGGAGCGATTTGTGCTGTCACCTTCTTCACTATCCTGC | |
| TTTCCTATGGGGTCATATTACACTCTCTTAAGACTCAGAGTTTGGTGGGAAACGAAAAGC | |
| TTTCTACACCTGTGCATCCCACGTCACTGTGGTCATTTATTCTTGTCCCCTGTATCTTCTT | |
| GTATGCAAGGCCCAATTCTACTTTTCCCATTGATAAATCCATGACTGTAGTTCTTCTTTTA | |
| TAACTCCCATGCTGAACCCACTAATCTATACCCTGAAGAATGCAGAAATGAAAAGTGCCAT | |
| GAGGAAACTTTGGAGTAAAAAAGTAAGCTTAGCTGGGATTGGCTGTATCACTGATGA | |
| AOLFR189 sequences: | |
| MQQNNSVPEFILLGLTQDPLRQMVFVIFLIFTMGTVVGNMLIIVTIKSSRTLGSPMTTFLFTLSF | (SEQ ID NO:349) |
| ADSCFSTSTARLIVALSEKMITTNECMTQTALHLFGCMEIFVLILRTAICKPLRYP | |
| TIMSQQVCIILIVLAWIGSLIIISTAQIILALRLPFCGPTLDHTCCDLQPLLTACMDTYMINLLL | |
| VSNSGAICSSSFMILIISTVILHSLRNHSAKGKKXALSACTSHIIILFFGPCIFIYTRPPTTFPMD | |
| KMVAVFTTIGTPFLNPLITTSEECRSEKCHEK | |
| ATGCAGCAAAATAACAGTGTGCCTGAATTCATACTGTTAGGATTTCACAGGATCCCTGA | (SEQ ID NO:350) |
| GGCAGAAAATAGTGTTTGTAATCTTCTTAATTTCTATATGGGTCTGTGGTGGGGTTAT | |
| GCTCATTATTGTGACCATCAAGTCCAGCCGGACACTAGGAAGCCCCATGTACTTCTTTCTA | |
| TTTTTATTTGTCCTTTGCAGATTCTTGCTTTTCAACTTCCACAGCCCCTAGATTTTTGTGGA | |
| TGCTCTCTCTGAAAAGAAAATTATAACCTACAATGAGTGCATGACACTGTCTTTGCACTA | |
| CATTTATTTTGGCTGCATGGAGATCTTTGTCCTCATTCTCATGGCTGTTGATCGCTATGTGGC | |
| CATCTGTAAGCCCTTGCGTTACCCAACCATCATGAGCCAGCAGGTCTGCATCATCCTGATT | |
| GTTCTTGCCTGGATAGGGTCTTTAATACACTCTACAGCTCAGATTATCCTGGCCTTTTGATT | |
| GCCTTTCTGTGGACCCTATTTGATTGATCATTATTGCTGTGATTTGCAGCCCTGTTGTTAC | |
| TTGCCTGCATGGACACTTACATGATGTCCTGCTGTTGGTGTCTTCAGTGGGGCTTTTTG | |
| CTCAAGTAGTTTCATGATTTTGATAATTTCATATATTGTCATCTTGCATCACTGAGATTCC | |
| ACAGTGCCAAAGGGAAGAAAAAGGCTCTCTCCGCTPGCACGTCTCACATTTTGTAGTCAT | |
| CTTATTCTTTTGGCCCATGTATATTCATATATACACGCCCCCCGACCACTTTCCCCATGGACA | |
| AGATGGTGGCAGTATTTTATACTATTGGAACACCCTTTCTCTTCCACTCATCTACACATCT | |
| GAGGAATGCAGAAGTGAAAAATGCCATGAGAAAG | |
| AOLFR19G sequences: | |
| MQRSNHTVTEFILLGFTTDPGMQLGLFVTLGVTCLTVVGSSTLIVLICNDSTHTPNTVIGN | (SEQ ID NO:351) |
| LSFLDLWTSSVHTPTLVTCISEDKSISFAGCLCQFFSARLATSECTLLTHTTAISPLL | |
| YAQTMPRRLCICLVLTSTTGGFVNAHLTSNTFTLDFCGDNVIDDFFCDVPPLVKLACSTSYQ | |
| AVLHFLLASNVISPTVLILASTLSIITTILRIHSTQGRFSTCSSHLISVTLTGSILTSTSSS | |
| YSLKLRDKMVSTFTTMLFPMLNIPMITSLRSKDMKDALKKFFKSA | |
| ATGGAGAGGAGCAATCACACAGTGACTGAGTTCATCCTGCTGGGCTTCACCACAGATCCAG | (SEQ ID NO:352) |
| GGATGCAACTGGGCCTCTTTGTGGTGTTCCTGGGTGTGTACTGTCTGACTGTGGTAGGTG | |
| TAGCACCCTGATCGTGTTGATCTGTAATGACTCCCGCCTACACACACCCATGTATTTTGTCA | |
| TTGGAAATCTGTCATTTCTGGATCTCTGGTATTCTTCTGTCCACACCGCATTGATCCTAGTG | |
| ACCTGCATCTCTGAAGACAAAAGCATCTCCTTTGCTGGCTGCCTGTGTCAGTTCTTGTCTGC | |
| CAGGCTGGCCTATAGTGAGTGCTACCTACTGGCTGCCATGGCTTATGACCACTACGTGGCC | |
| ATCTCCAAGCCCCTGCTTTATGCTCAGACCATGCCAAGGAGATTGTGCATCTGTTTGGTTTT | |
| ATATTCCTATACTGGGGGTTTTGTCAATGCAATAATATTAACCAGCAACACATTCACATTG | |
| GATTTTTGTGGTGACAATGTCATTGATGACTTTTTCTGTGATGTTCCACCCCTCGTGTGCT | |
| GGCATGCAGTGTGAGAGAGAGCTACCAGGCTGTGCTGCACTTCCTTCTGGCCTCCTTTGTC | |
| ATCTCCCGTACTGTGCTCATCCTTGCCTCTTACCTCTCCATCATCACCACCATCCTGAGGAT | |
| CCACTCTACCCAGGGCCGCATCAAAGTCTTCTCCACATGCTCCTCCCACCTGATCTCCGTTA | |
| CCTTATACTATGGCTCCATCTCTACTCTACTCCCGGCCTGTTCCAGCTACTCCCTCTG | |
| AGGGACAAAATGGTTTCTACCTTTATACTATGCTGTTCCCCATGTTGAATCCCATGATCTA | |
| CAGTCTGAGGAGTAAAGACATGAAAGACGCTCTGAAAAAATTCTTCTGTCAGCATT | |
| AOLFR191 sequences: | |
| MTGGGNITEITTTILLGFSDFPRIIKVLFTIFLVITTSLAWNLSLIVLIRMDSHLHTPMTTFLSNLS | |
| FIDVCTSSTVPKMLSNLLQEQQTTFVGCIIQTTIFSTMGLSESCLMTAMATDRTAAICNPLLYS | |
| SIMSPTLCVWMVLGATMTGLTASLFQIGALLQLHFCGSNVIPdTFFCDMPQLLILSCTDTFFVQV | |
| MTAILTMFFGIASALVIMISTGTGISIIMTTSAKGSPKAFNTCASHLLTAVSLFTTSGIFVTLRSSS | |
| GGSSSFDRFASVFTTVTPMLNPLITSLRNKEIKDALKRLQKRKCC | |
| ATGACTGGGGGAGGAAATATTACAGAAATCACCTATTTCATCCTGCTGGGATTCTCAGATT | (SEQ ID NO:354) |
| TTCCCAGGATCATAAAAGTGCTCTTCACTATATTCCTGGTGATCTACATTACATCTCTGGCC | |
| TGGAACCTCTCCCTCATTGTTTTAATAAGGATGGATTCCCACCTCCATACACCCATGTATTT | |
| CTTCCTCAGTAACCTGTCCTTCATAGATGTCTGCTATATCAGCTCCACAGTCCCCAAGATGC | |
| TCTCCAACCTCTTACAGGAACAGCAAACTATCACTTTTGTTGGTTGTATTATTCAGTACTTT | |
| ATCTTTTCAACGATGGGACTGAGTGAGTCTTGTCTCATGACAGCCATGGCTTATGATCGTT | |
| ATGCTGCCATTTGTAACCCCCTGCTCTATTCATCCATCATGTCACCCACCCTCTGTGTTTGG | |
| ATGGTACTGGGAGCCTACATGACTGGCCTCACTGCTTCTTTATTCCAAATTGGTGCTTTGCT | |
| TCAACTCCACTTCTGTGGGTCTAATGTCATCAGACATTTCTTCTGTGACATGCCCCAACTGT | |
| TAATCTTGTCCTGTACTGACACTTTCTTTGTACAGGTCATGACTGCTATATTAACCATGTTC | |
| TTTGGGATAGCAAGTGCCCTAGTTATCATGATATCCTATGGCTATATTGGCATCTCCATCA | |
| TGAAGATCACTTCAGCTAAAGGCAGTCCAAAGGCATTCAACACCTGTGCTTCTCATCTAAC | |
| AGCTGTTTCCCTCTTCTATACATCAGGAATCTTTGTCTATTTGAGGTCCAGCTCTGGAGGTT | |
| CTTCAAGCTTTGACAGATTTGCATCTGTTTTCTACACTGTGGTCATTCCCATGTTAAATCCC | |
| TTGATTTACAGTTTGAGGAACAAAGAAATTAAAGATGCCTTAAAGAGGTTGCAAAAGAGA | |
| AAGTGCTGCTGA | |
| AOLFR192 sequences: | |
| MENNTEVTEFILVGLTDDPELQIPLFIVFLFITLITLVGNLGMIELTLLDSCLHTPMTTFLSNLSLV | (SEQ ID NO:355) |
| DFGTSSAVTPKVMVGFLTGDTFILTNACATQFFFFVAFITAESFLLASMATDRTAALCKPLHY | |
| TTTMTTNVCACLAIGSTCGFLNASIHTGNTFRLSFCRSNVVETFFCDAPPLLTLSCSDNTSEM | |
| VIFFVVGFNDLFSILVILISTLFIFITIIVIKMRSPEGRQKAFSTCASHLTAVSIFTGTGIFMTLRPNSS | |
| HFMGTDKMASVFTAIVIPMLNPLVTSLRNKEVKSAFKKTVGKAKASIGFIF | |
| ATGGAGAACAACAGAGAGGTGACTGAATTCATCCTTGTGGGGTTAACTGATGACCCAGAA | (SEQ ID NO:356) |
| CTGCAGATCCCACTCTTCATAGTCTTCCTTTTCATCTACCTCATCACTCTGGTTGGGAACCT | |
| GGGGATGATTGAATTGATTCTACTGGACTCCTGTCTCCACACCCCCATGTACTTCTTCCTCA | |
| GTAACCTCTCCCTGGTGGACTTTGGTTATTCCTCAGCTGTCACTCCCAAGGTGATGGTGGG | |
| GTTTCTCACAGGAGACAAATTCATATTATATAATGCTTGTGCCACACAATTCTTCTTCTTTG | |
| TAGCCTTTATCACTGCAGAAAGTTTCCTCCTGGCATCAATGGCCTATGACCGCTATGCAGC | |
| ATTGTGTAAACCCCTGCATTACACCACCACCATGACAACAAATGTATGTGCTTGCCTGGCC | |
| ATAGGCTCCTACATCTGTGGTTTCCTGAATGCATCCATTCATACTGGGAACACTTTCAGGC | |
| TCTCCTTCTGTAGATCCAATGTAGTTGAACACTTTTTCTGTGATGCTCCTCCTCTCTTGACT | |
| CTCTCATGTTCAGAGAACTACATGAGTGAGATGGTTATTTTTTTGTGGTGGGATTCAATG | |
| ACCTCTTTTCTATCCTGGTAATCTTGATCTCCTACTTATTTATATTTATCACCATCATGAAG | |
| ATGCGCTCACCTGAAGGACGCCAGAAGGCCTTTTCTACTTGTGCTTCCCACCTTACTGCAG | |
| TTTCCATCTTTTATGGGACAGGAATCTTTATGTACTTACGACCTAACTCCAGCCATTTCATG | |
| GGCACAGACAAAATGGCATCTGTGTTCTATGCCATAGTCATTCCCATGTTGAATCCACTGG | |
| TCTACAGCCTGAGGAACAAAGAGGTTAAGAGTGCCTTTAAAAAGACTGTAGGGAAGGGAA | |
| AGGCCTCTATAGGATTCATATTTTAA | |
| AOLFR193 sequences: | |
| MENKTEVTQFILLGLTNDSELQVPLFITFPFITITLVGNLGIIVLIFWDSCLHINPMTTFLSNLSLV | (SEQ ID NO:357) |
| DFCTSSAVTPIVMAGFLIEDKVISTNACAAQMTFVAFATVENTLLASMATDRTAAVCKPLHY | |
| TTTMTTTVCARLAIGSTLCGFLNASIHTGDTFSLSFCKSNEVHHFFCDIPAVMVLSCSDRHISEL | |
| VLIYVVSFNIFIALLVILISTTFIFITILKMHSASVTQKPLSTCASHFIAVGIFTGTIIFMTLQPSSSH | |
| SMDTDKMAPVFTTMVIPMLNPLVTSLRNKEVKSAFKKVVEKAKLSVGWSV | |
| ATGGAAATAAGACAGAAGTAACACAATTCATTCTTCTAGGACTTCCTTGACTCAGT | (SEQ ID NO:358) |
| CTGCAGGTTCCCCTCTTTATAACGTTCCCCTTCATCTATATTATCACTCTGGTTGGATCCT | |
| GGGAATTATTGTATTGATATTCTGGGATTCCTGTCTCCACTTCCCATGTACTTTTTTCTCA | |
| GTAACTTGTCTCTAGTGGACTHTGCTACTCTTCAGCTGTCACTCCCATCGTCATGGCTGGA | |
| TTCCTTATAGAAGACAAGGTCATCTCTTACAATGCATGTGCTGCTCTATGTATATCTTTGT | |
| AGCTTTTGGCACTGTGGAAAATTACCTCTTGGCCTCTTGGCCTATGACCGGTATGCAGCA | |
| GTGTGCAACCCCTACATTACACCACAACCATGACAACAACTGTGTGTGCTCGTCTGGCCA | |
| TAGGCTCCTACCTCTGTGGTTTCCTGAATGCCTGCATCCACACTGGGGACACATTTAGTCTC | |
| TCTTTCTGTAAGTCCAATGAAGTCCATCACTTTTTCTGTGATATTCCAGCAGTCATGGTTCT | |
| CTCTTGCTCTGATAGACATATTAGCGAGCTTGTTCTTATTATGTTGTGAGCTTCTTTATCT | |
| TTATAGCTCTCCTGGTTATCTTGATATCCTACACATTCATTTTTATCACCATCCTATTGATG | |
| CACTCAGCTTCAGTATACCAGAAGCCTTTGTCCACCTGTGCCTCTCATTTCATGCAGTCGG | |
| CATCTTCTATGGGACTATTATCTTCATGTACTTACAACCCAGCTCCAGTCACTCCATGGACA | |
| CAGACAAAATGGCACCTGTGTTCTATACAATGGTCATCCCCATGGTGTCCCTCTGGTCTA | |
| TAGTCTGAGGAACAAGGAAGTGAAGAGTGCATTCAAGAGTTGTTGAGTGGCTTTTT | |
| GTCTGTAGGATGGTCAGTTTAA | |
| AOLFR194 sequences: | |
| MERQNQSCVVEFILLGFSNTPELQGQLFVAFLVITLVTLIGNAIIIVIVSLDQSLHTMTLFLLNL | (SEQ ID NO:359) |
| SVVDLSFSAVIMPEMLVVLSTEKTTISFGGCFAQMTTILLFGGAECFLLGlTICHPL | |
| NYQMIMNKGVFMKLIIFSWALGFMLGTVQTSWVSSFPFCGLNEETISCETPAVLELACITTFL | |
| FEIYAFTGTFLIILVPFLLILLSTTVLFAILKMITSTTGRQKMSTCATTLTSVTLFTGTASMTYLQ | |
| PKSGTSPETKKVMSLSTSLLTPLLNLLITSLRNSEMKRALMKLWTTTlTIHTI | |
| ATGGAAAGACAAAATCAAAGCTGTGTGGTTGAATTCATCCTCTTGGGCTTTTCTTTCTATC | (SEQ ID NO:360) |
| CTGAGCTCCAGGGGCAGCTCTTTGTGGCTTTCCTGGTTATTATCTGGTGACCCTGATAGG | |
| AAATGCCATTATTATAGTCATCGTCTCCCTAGACCAGAGCCTCCACGTTCCCATGTACCTGT | |
| TTCTCCTGAACTTATCTGTGGTGGACCTGAGTTTCAGTGCAGTTATTATGCCTGATTGCT | |
| GGTGGTCCTCTCTACTGAAAAAACTACAATTTCTTTTGGGGGCTGTTTTGCACAGATGTAT | |
| TTCATCCTTCTTTTTGGTGGGGCTGAATGTTTTCTTCTGGGAGCTTGGCTTATGACCGATT | |
| TGCTGCAATTTGCCATCCTCTCAACTACCAAATGATTATGAATATGGAGTTTTATGAT | |
| TTAATTATATTTTCATGGGCCTTAGGTTTTATGTTAGGTACTGTTCTTACATCATGGGTATC | |
| TAGTTTTCCCTTTTGTGGCCTTAATGAAATTAACCATATATCTTGTGATTCCCCAGCAGTGT | |
| TAGAACTTGCATGTGCAGACACGTTTTTGTTTGAAATCTATGCATTCACAGGCACCTTTTTG | |
| ATTATTTTGGTTCCTTTCTTGTTGATACTCTTGTCTTACATTCGAGTTCTGTTGCCATCCTG | |
| AAGATGCCATCAACCACTGGGAGACAAAAGGCCTTTTCCACCTGTGCCGCTCACCTCACAT | |
| CTGTGACCCTATTCTATGGCACAGCCAGTATGACTTATTTACAACCCTTATCTGGCTACTC | |
| ACCGGAAACCAAGAAAGTGATGTCATTGTCTTACTCACTTCTGACACCACTGCTGTTCTG | |
| CTTATCTACAGTTTGCGTATAGTGAGATGAAGAGGGCTTTGATGTTTTATGGCGAAGG | |
| CGAGTGGTTTTACACACAATCTGA | |
| AOLFR195 sequences: | |
| MIVQLICTVCFLAVNTFHVRSSFDFLKDDMGEINQTLVSEFLLLGLSGEIWTTALILVMY | (SEQ ID NO:361) |
| LVILIGNGVLIIASIFDSHFHTPMTTFLGNLSFLDICTTSSSVPSTLVSLISKTKRNISFSGCAVQMFF | |
| GFAMGSTECLLLGMEDRTVAICNPLRTPIILSKVATVLMASVSWLSGGTSAVQTLLAMRL | |
| PFCGNMTNHFACEILAVKLACADISLNIITMVISNMFLVLPLMVIFFSWFILTTILQTSATG | |
| RRKAFSTCSAHLTVVIIFTGTIFFMTAKPKSQDLIGEEKLQALDKLISLFTGVVTPMLNPILTSLR | |
| NKDVKAAVKTLLNKKPIH | |
| ATGATTGTTCAGTTAATTTGTACTGTTTGTTTCTTGGCAGTTTTACATTTCATGTTAGATC | (SEQ ID NO:362) |
| TCTTTTGATTTGCTGAAAGCAGATGACATGGGTGAGATTAACCAGACACTTGTGTCAGTT | |
| TTTCTTCTTCTGGGTCTTTCTGGATACCCAAAGATTGAGATTGTTTACTTTGCTCTCATTCT | |
| AGTTATGTACCTAGTGATTCTAATTGGCAATGGTGTTCTAATCATAGCCAGCATCTTTGATT | |
| CTCATTTTCACACACCAATGTACTTCTTCCTGGGCAACCTCTCTTTCCTGGATATCTGCTAT | |
| ACATCCTCCTCTGTTCCCTCAACATTGGTGAGCTTAATCTCAAAGAAAAGATCATTTCCT | |
| TCTCTGGATGTGCAGTGCAGATGTTCTTTGGGTTTGCAATGGGGTCAACAGAATGTCTGCT | |
| TCTTGGCATGATGGCATTTGATCGTTATGTGGCCATCTGCAACCGACTGAGATACCCCATC | |
| ATCCTGAGCAAGGTGGCGTATGTATTGATGGCTTCTGTGTCCTGGCTGTCCGGTGGAATAA | |
| ATTCAGCTGTGCAAACATTACTTGCCATGAGACTGCCTTTCTGTGGGAATAATATTATCAA | |
| TCATTTCGCATGTGAAATATTAGCTGTCCTCAAGCTGGCCTGTGCTGATATATCCCTCAATA | |
| TTATCACCATGGTGATATCAAATATGGCCTTCCTGGTTCTTCCACTGATGGTCATTTTTTTC | |
| TCCTATATGTTCATCCTCTACACCATCTTGCAAATGAATTCAGCCACAGGAAGACGCAAGG | |
| CATTTTCCACGTGCTCAGCTCACCTGACTGTGGTGATCATATTTTACGGTACCATCTTCTTT | |
| ATGTATGCGAAACCGAAGTCTCAAGACCTGATTGGGGAAGAAAAATTGCAAGCATTAGAC | |
| AAGCTCATTTCTCTGTTTTATGGGGTAGTGACACCCATGCTGAATCCTATACTCTATAGCTT | |
| GAGAAATAAGGATGTAAAAGCTGCTGTAAAATATTTGCTGAACAAAAAACCAATTCACTA | |
| A | |
| AOLFR196 sequences: | |
| MLESNTTMPTEFLFVGFTDTLPLRVTLFLVFLLVTTLTMVGNILLIILVMNSSLQTPMTTTLSNL | (SEQ ID NO:363) |
| SFLDISCSTATTPKMLANFLASRKSISPTGCALQMFFFASFADAECLILAAMATDRTAAICNPLL | |
| YTTLMSRRVCVCFIVLATPSGSTTSLVHVCLTFRLSFCGSNIVNHFFCDIPPLLALSCTDTQINQL | |
| LLFALCSFIQTSTFVVIFISTTCILITVLSIXSSGGRSKTFSTCASHLIAVTLFTGALLFMTLQPTTS | |
| YSLDTDKVVAVFTTVVFPMFNPIITSFRNKDVKNALKKLLERIGTSNEWTLNRLRIVNI | |
| ATGTTGGAGAGTAATTACACCATGCCAACTGAGTTCCTATTTGTTGGATTCACAGATTATC | (SEQ ID NO:364) |
| TACCTCTCAGAGTCACACTGTTCTTGGTATTCCTTCTGGTATATACATTAACTATGGTCGGA | |
| AATATACTCTTAATAATTCTAGTTAATATTAATTCAAGCCTTCAAATTCCCATGTATTATTT | |
| TCTTAGCAACTTATCTTTCTTAGACATCAGCTGTTCTACAGCAATCACTCCTAAAATGCTGG | |
| CAAACTTGTTGGCATCCAGGAAAAGCATCTCTCCTTATGGGTGTGCACTACAAATGTTTTT | |
| CTTCGGTTCTTTTGCTGATGCTGAGTGCCTTATCCTGGCAGCAATGGCTTATGACCGCTATG | |
| CAGCCATCTGCAACCCACTGCTCTATACTACACTGATGTCTAGGAGAGTGTGTGTCTGCTT | |
| CATTGTGTTGGCATATTTCAGTGGAAGTACAACATCACTGGTCCATGTGTGCCTCACATTC | |
| AGGCTGTCATTTTGTGGCTCCAATATCGTCAATCATTTTTTCTGTGATATCCCACCTCTTCT | |
| GGCTTTATCATGTACAGACACTCAGATCAACCAGCTTCTGCTCTTIITGCTTTGTGCAGCTTCA | |
| TCCAGACCAGCACTTTTGTGGTAATATTTATTTCTTACTTCTGCATCCTCATCACTGTGTTG | |
| AGCATCAAGTCCTCAGGTGGCAGAAGCAAAACATTCTCCACTTGTGGTTCCCACCTCATAG | |
| CAGTCACCTTATTCTATGGAGCGCTCGTGTTTATGTACTTACAGCCCACCACTAGCTATTCC | |
| CTAGACACTGATAAGGTGGTGGCAGTGTTTATACTGTTGTATTTCCCATGTTTAATCCAA | |
| TAATTTATAGTTTCAGAAACAAGGATGTGAAAAATGCTCTCAAAAAGCTATTAGAAAGAA | |
| TTGGATATTCAAATGAATGGTATTTAAATCGTTTAAGAATAGTCAATATCTAA | |
| AOLFR197 sequences: | |
| MCYLSQLCLSLGEIITLHMGMVRHTNESNLAGFILLGFSDTPQLQKVLFVLILILTLLTTILGNTTI | (SEQ ID NO:365) |
| ILVSRLEPKLHMPMTTFLSHLSFLTRCFTSSVIPQLLVNLWEPMKTIATGGCLVHLTNSHALGS | |
| TECVLLALMSCDRTVAVCRPLHTTVLMHIHLCMALASMAWLSGLATTLVQSTLTLQLPFCGH | |
| RQVDHFICEVPVLIKLACVGTTFNEAELFVASILFLIVPVSFILVSSGTAHAVLRIKSATRRQKAF | |
| GTCFSHLTVVTIFTGTIIFMTLQPAKSRSRDQGKFVSLFTTVVTRMLNPLITTLRIKEVKGALKK | |
| VLAKALGVNIL | |
| ATGTGTTATCTTTCTCAGCTATGCCTCAGCCTTGGGGAACACACTTTACATATGGGGATGG | (SEQ ID NO:366) |
| TGAGACATACCAATGAGAGCAACCTAGCAGGTETTCATCCTTTTTAGGGTTTTCTGATTATCC | |
| TCAGTTACAGAAGGTTGTATTTGTGCTCATATTGATTCTGTATTTACTAACTATTTTGGGGA | |
| ATAGCACCATCATTCTGGTTTCTCGTCTGGAACCCAAGCTTCATATGCCGATGTATTTCTTC | |
| CTTTCTCATCTCTCCTTCGTGTACCGCTGCTTCACCAGCAGTGTTATTCCCCAGGTCCTGGT | |
| AAACCTGTGGGAACCCATGAAAACTATCGCCTATGGTGGCTGTTTGGTTCACCTTTACAAC | |
| TCCCATGCCCTGGGATCCACTGAGTGCGTGCTCTTGGCTCTGATGTCCTGTGACCGCTATGT | |
| GGCTGTCTGCCGTCCTCTCCATTACACTGTCTTAATGCATATCCATCTCTGCATGGCCTTGG | |
| CATCTATGGCATGGCTCAGTGGAATAGCCACCACCCTGGTACAGTCGACCCTCACCCTGCA | |
| GCTGCCCTTCTGTGGGCATCGCCAAGTGGATCATTTCATCTGCGAGGTCCCTGTGCTCATC | |
| AAGCTGGCTTGTGTGGGCACCACGTTTAACGAGGCTGAGCTTTTTGTGGCTAGTATCCTTT | |
| TCCTTATAGTGCCTGTCTCATTCATCCTGGTCTCCTCTGGCTACATTGCCCACGCAGTGTTG | |
| AGGATTAAGTCAGCTACCAGGAGACAGAAAGCATTTCGGGACCTGCTTCTCCCACCTGACA | |
| GTGGTCACCATCTTTTATGGAACCATCATCTTCATGTATCTGCAGCCAGCCAAGAGTAGAT | |
| CCAGGGACCAGGGGAAGTTTGTTTCTCTCTTCTACACTGTGGTAACCCGCATGCTTAACCC | |
| TCTTATTTATACCTTGAGGATCAAGGAGGTGAAAGGGGCATTAAAGAAAGTTCTAGCAAA | |
| GGCTCTGGGAGTAAATATTTTATGA | |
| AOLFRL98 sequences: | |
| MENCTEVTKFILLGLTSVPELQIPLFILFTFITLLTLCGNLGMMLLILMDSCLHTPMTTFLSNLSL | (SEQ ID NO:367) |
| VDFGTSSAVTPKVMAGFLRGDKVISTNACAVQMFFFVALATVENTLLASMATDRTAAVCKP | |
| LHYTTTMTASVGACLALGSTVCGFLNASFIIIGGIPSLSFCKSNLVHHFFCDVPAVMALSCSDKH | |
| TSEVILVFMSSFNIFFVLLVIFISTLFIFITILKMHSAKGHQKALSTCASHFTAVSVFTGTVIFIYLQ | |
| PSSSHSMDTDKMASVFTAMIIPMLNPVVTSLRNREVQNAFKKVLRRQKFL | |
| ATGGAGAATTGTACGGAAGTGACAAAGTTCATCTTCTAGGACTAACCAGTGTGCCAGAAC | (SEQ ID NO:368) |
| TACAGATCCCCCTCTTTATCTTGTTCACCTTCATCTACCTCCTCACTCTGTGTGGGAACCTG | |
| GGGATGATGTTGCTGATCCTGATGGACTCTTGTGTCGACACCCCGATGTACTTTTTCCTCAG | |
| TAACCTGTCTCTGGTGGACTTTGGATACTCCTCAGCTGTCACTCCCAAGGTCATGGCTGGG | |
| TTCCTTAGAGGAGACAAGGTCATCTCCTACAATGCATGTGGTGTTCAGATGTTCTTCTTTGT | |
| AGCCTTGGCCACGGTGGAAAATTACTTGTTGGCCTCAATGGCCTATGACCGCTATGCAGCA | |
| GTGTGCAAACCCCTACACTACACCACCACCATGACGGCCAGTGTAGGTGCCTGTCTGGCCC | |
| TAGGCTCATATGTCTGTGGCTTCCTAAATGCCTCATTCCACATTGGGGGCATATTCAGTCTC | |
| TCTTTCTGTAAATCCAATCTGGTACATCACTTTTTCTGTGATGTTCCAGCAGTCATGGCTCT | |
| GTCTTGCTCTGATAAACACACTAGTGAGGTGATTCTGGTTTTTATGTCAAGCTTTAATATCT | |
| TTTTTGTTCTTCTAGTTATCTTTATCTCCTACTTGTTCATATTCATCACCATCTTGAAGATGC | |
| ATTCAGCTAAGGGACACCAAAAAGCATTGTCCACCTGTGCCTCTCACTTCACTGCAGTCTC | |
| CGTCTTCTATGGGACAGTAATCTTCATCTACTTGCAGCCCAGCTCCAGCCACTCCATGGAC | |
| ACAGACAAAATGGCATCTGTGTTCTATGCTATGATCATCCCCATGCTGAACCCTGTGGTCT | |
| ACAGCCTGAGGAACAGAGAAGTCCAGAATGCATTCAAGAAAGTGTTGAGAAGGCAAAAAT | |
| TTCTATAA | |
| AOLFR199 sequences: | |
| MDTGNKTLPQDFLLLGFPGSQTLQLSLFMLFLVMTLTVSGNVAILMLVSTSHQLHTPMYFFLS | (SEQ ID NO:369) |
| NLSFLEIWTTTAAVPKALAILLGRSQTISFTSCLLQMTTVFSLGCTETTLLAAMATDRCLAICYP | |
| LHTGATMSSLLSAQLALGSWVCGFVAIAVPTALISGLSFCGPRMNHFFCDIAPWIALACTNTQA | |
| VELVAFVIAVVVILSSCLITFVSTVTISTILRIIPSASGRSKAFSTCSSLILTVVLIWTGSTVFLHVR | |
| TSIKDALDLIKAVHVLNTVVTPVLNPFITTLRTTKEVRETLLKKWKGK | |
| ATGGACACAGGCAACAAAACTCTGCCCCAGGACTTTCTCTTACTGGGCTTTCCTGGTTCTC | (SEQ ID NO:370) |
| AAACTCTTCAGCTCTCTCTCTTTATGCTTTTTCTGGTGATGTACATCCTCACAGTTAGTGGT | |
| AATGTGGCTATCTTGATGTTGGTGAGCACCTCCCATCAGTTGCATACCCCCATGTACTTCTT | |
| TCTGAGCAACCTCTCCTTCCTGGAGATTTGGTATACCACAGCAGCAGTGCCCAAAGCACTG | |
| GCCATCCTACTGGGGAGAAGTCAGACCATATCATTTACAAGCTGTCTTTTGCAGATGTACT | |
| TTGTTTTCTCATTAGGCTGCACAGAGTACTTCCTCCTGGCAGCCATGGCTTATGACCGCTGT | |
| CTTGCCATCTGGTATCCTTTACACTACGGAGCCATCATGAGTAGCCTGCTCTCAGCGCAGC | |
| TGGGCCTGGGCTCCTGGGTGTGTGGTTTCGTGGCCATTGCAGTGCCCACAGCCCTCATCAG | |
| TGGCGTGTCCTTCTGTGGCCCCCGTGCCATCAACCACTTCTTCTGTGACATTGCACCCTGGA | |
| TTGCCCTGGCCTGCACCAACACACAGGCAGTAGAGCTTGTGGCCTTTGTGATTGCTGTTGT | |
| GGTTATCCTGAGTTCATGCCTCATCACCTTTGTCTCCTATGTGTACATCATCAGCACCATCC | |
| TCAGGATCCCCTCTGCCAGTGGCCGGAGCAAAGCCTTCTCCACGTGCTCCTCGCATCTCAC | |
| CGTGGTGCTCATTPGGTATGGGTCCACAGTTTTCCTTCACGTCCGCACCTCTATCAAAGAT | |
| GCCTTGGATCTGATCAAAGCTGTCCACGTCGTGAACACTGTGGTGACTCCAGTTTTAAACC | |
| CCTTCATCTATACGCTTCGTAATAAGGAAGTAAGAGAGACTCTGCTGAAGAAATGGAAGG | |
| GAAAATAA | |
| AOLFR200 sequences: | |
| MTRKINTTSLTEFVLLGLADTLELQIILFLFFLVITTLTVLGNLGMILLIRIDSQLHTPMTTFLANL | (SEQ ID NO:371) |
| SFVDVCNSTTTPKMLADLLSETKTISFAGCFLQMTTFISLATTECILFGLMATDRTAAICRPLL | |
| YSLIMSRTVTLKMAAGAFAAGLLNFMVNTSHVSSLSFCDSNVIHHFFCDSPPLFKLSCSDTILKE | |
| SISSILAGVNIVGTLLVILSSTSTVLFSIFSMHSGEGRHRAFSTCASHLTAIILFTATCITTTLRPSS | |
| SYSLNQDKVASVFTTVVIPMLNPLITSLRSKEVKKALANVISRKRTSSFL | |
| ATGACCAGAAAAAATTATACCTCACTGACTGAGTTCGTCCTATTGGGATTAGCAGACACGC | (SEQ ID NO:372) |
| TGGAGCTACAGATATCCTCTTTTTGTTTTTTCTTGTGATTTATACACTTACAGTACTGGGA | |
| AATCTCGGGATGATCCTCTTAATCAGGATCGATTCCCAGCTTCAGACACCCATGTATTTCTT | |
| CCTGGCTAACCTGTCCTTTGTGGACGTTTGTAACTCAACTACCATCACCCCAAAGATGCTG | |
| GCAGATTTATTATCAGAGAAGAAAACCATGTCTTTGCTGGCTGCTTCCTACAGATGTACT | |
| TCTTTATCTCCCTGGCGACAACCGAATGCATCCTCTTTTGGGTTAATGGCCTATGACAGGTA | |
| TGCGGCCATATGTCGCCCGCTGCTTTACTCCTTGATCATGTCCAGGACGGTCTACCTAAAA | |
| ATGGCAGCCGGGGCTTTTGCTGCAGGGTTGCTGAACTTCATGGTCAACACAAGCCATGTCA | |
| GCAGCTTGTCATTCTGTGACTCCAATGTCATCCATCACTTCTTCTGTGAGAGTCCCCCACTT | |
| TTCAAGCTCTCTTGTTCTGACACAATCCTGAAAGAAAGCATAAGTTCTATTTTGGCTGGTG | |
| TGAATATTGTGGGGACTCTGCTTGTCATCCTCTCCTGCTACTCCTACGTTCTCTTCTCCATT | |
| TTLTTTCTATGCATTCGGGGGAGGGGAGGCACAGAGCTTTCTCCACGTGTGCCTCTCACCTGA | |
| CAGCCATAATTCTGTTCTATGCCACCTGCATCTATACTTACCTGAGACCTAGTTCCAGCTAC | |
| TCCCTGAATCAGGACAAAGTGGCTTCTGTGTTCTACACAGTGGTGATTCCCATGTTGAATC | |
| CTCTGATCTACAGCCTCAGGAGTAAGGAAGTAAAGAAGGCTTTAGCGAATGTAATTAGCA | |
| GGAAAAGGACCTCTTCCTTTCTGTGA | |
| AOLFR201 sequences: | |
| MEWENHTILVEFFLKGLSGHLPRLELLFFVLIFIMTVVILLGNGTLILISILDPHLHTPMTTFLGNL | (SEQ ID NO:373) |
| SFLDICTTTTSIPSTLVSFLSERKTISLSGCAVQMFLGLAMGTFECVLLGMMAFDRTVAICNPLR | |
| YPIIMSKDATVPMAAGSWIIGAVNSAVQSVFVVQLPFCRNNIINIHFTCEILAVMKLACADISDN | |
| EFIMLVATTLFILTPLLLITVSTTLIIVSIFTSSSEGRSKASSTCSAIHLTVVIIFTGTILFMTMKPKS | |
| KETLNSDDLDATDTISMETGVMTPMMNPLITSLRNKDVKEAVTILLTTIRRFFSK | |
| ATGGAATGGGAAAACCACACCATTCTGGTGGAATTTTTTCTGAAGGGACTTTCTGGTCACC | (SEQ ID NO:374) |
| CAAGACTTGAGTTACTCTTTTTTGTGCTCATCTTCATAATGTATGTGGTCATCCTTCTGGGG | |
| AATGGTACTCTCATTTTAATCAGCATCTTGGACCCTCACCTTGACACCCCTATGTACTTCTT | |
| TCTGGGGAACCTCTCCTTCTTGGACATCTGCTACACCACCACCTCTATTCCCTCCACGCTAG | |
| TGAGCTTCCTTTTCAGAAAGAAAGACCATTTCCCTTTCTGGCTGTGCAGTGCAGATGTTCCT | |
| CGGCTTGGCCATGGGGACAACAGAGTGTGTGCTTCTGGGCATGATGGCCTTTGACCGCTAT | |
| GTGGCTATCTGCAACCCTCTGAGATATCCCATCATCATGAGTAAGGATGCCTATGTACCCA | |
| TGGCAGCTGGGTGCTGGATCATAGGAGCTGTCAATTCTGCAGTACAATCAGTGTTTGTGGT | |
| ACAATTGCCTTTCTGCAGGAATAACATCATCAATCATTTCACCTGTGAAATTCTGGCTGTC | |
| ATGAAACTGGCCTGTGCTGACATCTCAGACAATGAGTTCATCATGCTTGTGGCCACAACAT | |
| TGTTCATATTGACACCTTTTGTTATTAATCATTGTCTCTTACACGTTAATCATTGTGAGCATC | |
| TTCAAAATTAGCTCTTCCGAGGGGAGAAGCAAAGCTTCCTCTACCTGTTCAGCCCATCTGA | |
| CTGTGGTCATAATATTGTATGGGACCATCCTCTTCATGTACATGAAGCCCAAGTCTAAAGA | |
| GACACTTAATTCGGATGACTTGGATGCTACCGACAAAATTATATCCATGTTCTATGGGGTG | |
| ATGACTCCCATGATGAATCCTTTAATCTACAGTCTTAGAAACAAGGATGTGAAAGAGGCA | |
| GTAAAACACCTACTGAACAGAAGGTTCTTTAGCAAGTGA | |
| AOLFR202 sequences: | |
| MEWENHTILVEFFLKGLSGHPRLELLFFVLIFIMTVVILLGNGTLILISILDPHLHTPMTTFLGNL | (SEQ ID NO:375) |
| SFLDICTTTTSIPSTLVSFLSERKTISLSGCAVQMFLSLAMGTTECVLLGVMAFDRTVAICNPLR | |
| YPIIMSKDATVPMAAGSWIIGAVNSAVQTVFVVQLPFCRNNHNHTTCEILAVMTACADISGN | |
| EFILLVTTTLFLLTPLLLIIVSTTLIILSIFTSSSEGRSKPSSTGSARLTVVITFCGTIFLMTMKPKSQ | |
| ETLNSDDLDATDKLIFIFTRVMTPMMNPLITSLRNKDVKEAVKHLLRRKNFNK | |
| ATGGAATGGGAAAACCACACCATTCTGGTGGAATTTTTTCTGAAGGGACTTTCTGGTCACC | (SEQ ID NO:376) |
| CAAGACTTGAGTTACTCTTTTTTGTGCTCATCTTCATAATGTATGTGGTCATCCTTCTGGGG | |
| AATGGTACTCTCATTTTAATCAGCATCTTGGAGCCTCACCTTCACACCCCTATGTACTTCTT | |
| TCTGGGGAACCTCTCCTTCTTGGACATCTGCTACACCACCACCTCTATTCCCTCCACGCTAG | |
| TGAGCTTCCTTTCAGAAAGAAAGACCATTTCCCTTTCTGGCTGTGCAGTGCAGATGTTCCT | |
| CAGCTTGGCCATGGGGACAACAGAGTGTGTGCTTCTGGGCGTGATGGCCTTTGACCGCTAT | |
| GTGGGTATCTGCAACCCTCTGAGATATCCCATCATCATGAGTAAGGATGCCTATGTACCCA | |
| TGGCAGCTGGGTCCTGGATCATAGGAGCTGTCAATTCTGCAGTACAAACAGTGTTTGTGGT | |
| ACAATTGCCTTTCTGCAGGAATAACATCATCAATCATTTCACCTGTGAAATTCTAGCTGTC | |
| ATGAAACTGGCCTGTGCTGACATCTCAGGCAATGAGTTCATCCTGCTTGTGACCACAACAT | |
| TGTTCCTATTGACACCTTTGTTATTAATTATTGTCTCTTACACGTTAATCATTTTGAGCATC | |
| TTCAAAATTAGCTCTTCGGAGGGGAGAAGCAAACCTTCCTCTACCTGCTCAGCTCGTCTGA | |
| CTGTGGTGATAACATTCTGTGGGACCATCTTCCTCATGTACATGAAGCCCAAGTCTCAAGA | |
| GACACTTAATTCAGATGACTTGGATGCCACTGACAAACTTATATTCATATTCTACAGGGTG | |
| ATGACTCCCATGATGAATCCTTTAATCTACAGTCTTAGAAACAAGGATGTGAAGGAGGCA | |
| GTAAAACACCTACTGAGAAGAAAAAATTTTAACAAGTAA | |
| AOLFR203 sequences: | |
| MKRQNQSGVVEFILLGFSNFPELQVQLFGVFLVITVVTLMGNAIITVIISLNQSLHVPMTLFLLN | (SEQ ID NO:377) |
| LSVVEVSFSAVITPEMLVVLSTEKTMISFVGCFAQMTTILLFGGTECFLLGAMATDRFAAICHPL | |
| NYPVIMNRGVFMKLVIFSWISGIMVATVQTTWVFSFPFCGPNEThTHLFCETPPVLELVCADTFLF | |
| EIYAFTGTILIVMVPFLLILLSTRVLFAILKMPSTTGRQKAFSTCASHLTSVTLFTGTANMTYLQ | |
| PKSGTSPETKKLISLATTLLTPLLNPLITSLRNSEMKRTLIKLWRRLKVILHTF | |
| ATGAAAAGACAAAATCAAAGCTGTGTGGTTGAATTCATCCTCCTGGGCTTTTCTAACTTTC | (SEQ ID NO:378) |
| CTGAGCTCCAGGTGCAGCTCTTTGGGGTTTTCCTAGTTATTTATGTGGTGACCCTGATGGG | |
| AAATGCCATGATTACAGTCATCATCTCCTTAAACGAGAGCCTCCACGTTCCCATGTACCTGT | |
| TCCTCCTGAACCTATCTGTGGTGGAGGTGAGTTTCAGTGCAGTCATTACGCCTGAAATGCT | |
| GGTGGTGCTCTCTACTGAGAAAACTATGATTTCTTTTGTGGGCTGTTTTGCACAGATGTAT | |
| TTCATCCTTCTTTTTGGTGGGACTGAATGTTTTCTCCTGGGAGCGATGGCTTATGACCGATT | |
| TGCTGCAATTTGCCATCCTCTGAACTACCCAGTGATTATGAACAGAGGGGTTTTTATGAAA | |
| TTAGTAATATTCTCATGGATCTCAGGGATCATGGTGGCTACTGTGCAGACCACTTGGGTAT | |
| TTAGTTTTCCATTTTGTGGCCCCAATGAAATTAATCATCTCTTCTGTGAGACTCCCCCGGTA | |
| CTAGAGCTTGTGTGTGCAGACACCTTCTTATTTGAAATCTATGCCTTCACAGGCACCATTTT | |
| GATTTGTTATGGTTCCTTTCTTGTTGATCCTCTTGTCTTACATTCGAGTTCTGTTTGCCATCCT | |
| GAAGATGCCATCAACTACTGGGAGACAAAAGGCCTTTTCCACCTGTGCCTCTCACCTCACA | |
| TCTGTGACCCTGTTCTATGGCACAGCCAATATGACTTATTTACAACCCAAATCTGGCTACTC | |
| ACCCGAAACCAAGAAACTGATCTCATTGGCTTACACGTTGCTTACCCCTCTGCTCAATCCG | |
| CTCATCTATAGCTTACGAAACAGTGAGATGAAGAGGACTTTGATAAAACTATGGCGAAGA | |
| AAAGTGATTTTAGACACATTCTGA | |
| AOLFR204 sequences: | |
| MEKKKNVTEFILIGLTQNPIMEKVTFVVFLVLTMITLSGNLLIVVTTTSQALSSPMTTFLTHLSL | (SEQ ID NO:379) |
| IDTVTSSSSAPKLIVDSFQEKTISFNGCMAQATAEHIFGATEIILLTVMACDCTVAICTPLNYTT | |
| IMISHSLCILLVAVAWVGGFLHATIQILFTVWLPFCGPNVIGHFMCDLTPLLKLVCIDTHTLGLFV | |
| AVNSGFICLLNFLILVVSTVIILRSLKNNSLEGRCKALSTGISHIIVVVLFFVPCIFVTLRSVTTLPI | |
| DKAVAVFTTMVVPMLNPVVTTLRNAEVKSAIRIKLWRKKVTSDND | |
| ATGGAGAAGAAAAAGAATGTGACTGAATTCATTTTAATAGGTCTTACACAGAACCCCATA | (SEQ ID NO:380) |
| ATGGAGAAAGTCACGTTTGTAGTATTTTTGGTTCTTTACATGATAACACTTTCAGGCAACC | |
| TGCTCATTGTGGTTACCATTACCACCAGCCAGGCTCTGAGCTCCCCCATGTACTTCTTCCTG | |
| ACCCACCTTTCTTTTGATAGACACAGTTTATTCTTCTTCTTCAGCTCCTAAGTTGATTGTGGA | |
| TTCCTTTCAAGAGAAGAAAATCATCTCCTTTAATGGGTGTATGGCTCAAGCCTATGCAGAA | |
| CACATTTTTGGTGCTACTGAGATCATCCTGCTGACAGTGATGGCCTGTGACTGCTATGTGG | |
| CCATCTGCAAACCTCTGAACTACACAACCATTATGAGCCACAGCCTGTGCATTCTCCTGGT | |
| GGCAGTGGCCTGGGTGGGAGGATTTCTTTCATGCTCTATTCAGATTCTCTTTACAGTATGG | |
| CTGCCCTTCTGTGGCCCCAATGTCATAGGCCACTTCATGTGTGACTTGTACCCATTGTTATT | |
| ACTTGTTTGCATAGACACTCATACCCTTGGTCTCTTTGTTGCTGTGTCAGTGGGTTTATGT | |
| GCTTATTATCTTCCTTATCTTGGTGGTATCCTATGTGATCATCTTGAGATCTTTTTAGAAC | |
| AATAGCTTGGAGGGGAGGTGTAAAGCCCTCTCCACCTGTATTTCTCACATCATAGTAGTTG | |
| TCTTATTCTTTGTGCCCTGTATATTTGTGTATCTGCGCTCAGTGACCACTCTGCCCATTGAT | |
| AAAGCTGTTGCTGTATTTTATACTATGGTGGTCCCAATGTTAAATCCCGTGGTCTACACAC | |
| TCAGAAATGCTGAGGTAAAAAGTGCAATAAGGAAGCTTGGAGTTTGTGACTTCAG | |
| ATAATGATTAA | |
| AOLFR205 sequences: | |
| MESENRTVIREFILLGLTQSQDIQLLVFVLVLIFTTIILPGNLIIFTIKSDPGLTTLTFLGTML | (SEQ ID NO:381) |
| DASYSFTVAPRMLVDFLSAKTISTRGCITQLFFLHFLGGGEGLLLKDRTTICTLHTT | |
| VMNPRTCYAMMLALWLGGFVHSIIQVVLILRLPFCGPNQLDNFFCDVPQVIKLACTDTFVVEL | |
| LMVFNSGLMTLLCFLGLLASTAVILCRIRGSSSEAKAMSTCIThIIVIFEMFGPGIFIYTRPFRA | |
| FPADKVVSLFHTVIFPLLNPVLTTLRNQEVKASMKKVFNKHIA | |
| ATGGAAAGCGAGAACAGAACAGTGATAAGAGAATTCATCCTCCTTGGTCTGACCCAGTCT | (SEQ ID NO:382) |
| CAAGATATTCAGCTCCTGGTCTTTGTGCTAGTTTTAATATTCTACTTCATCATCCTCCCTGG | |
| AAATTTTCTCATTATTTTCACCATAAAGTCAGACCCTGGGCTCACAGCCCCCCTCTATTTCT | |
| TTCTGGGCAACTTGGCCTTCCTGGATGCATCCTACTCCTTCACTGTGGCTCCCCGGATGTTG | |
| GTGGACTTGCTCTCTGCGAAGAAGATAATCTCCTACAGAGGCTGCATCACTCAGCTCTTTT | |
| TCTTGCACTTCCTTGGAGGAGGGGAGGGATTACTCCTTGTTGTGATGGCCTTTGACCGCTA | |
| CATCGCCATCTGCCGGCCTCTGCACTATCCTACTGTCATGTCCCTAGTCCTGCTATGCA | |
| ATGATGTTGGCTCTGTGGCTTGGGGGTTTTGTCCACTCCATTATCCAGGTGGTCCTCATCCT | |
| CCGCTTGCGTTTTTGTGGCCCAAACCAGCTGGACAACTTCTTCTGTGATGTCCCACAGGTC | |
| ATCTGCTGGCCTGCACCGACACATTGTGGTGGAGCTTCTGATGGTCTCTCAGTGGCC | |
| TGATGACAGTCCTGTGCTTTCTGGGGCTTCTGGCCTCCTATGCAGTCATCTTTGTCGCATA | |
| CGAGGGTCTTCTTCTGAGGCAAAAAACAAGGCCATGTCCACGTGCATCACCCATATCATTG | |
| TTATATTCTTCATGTTTGGACCTGGCATCTTCATCTACACGCGCCCCTTCAGGGCTTTCCCA | |
| GCTGACAAGGTGGTTTCTCTCTTCCACACAGTGATTTTTTTCCTTTGTTGTTCCTGTCATTTA | |
| TACCCTTCGCAACCAGGAAGTGAAAGCTTCCATGGGTGTTTTTTGCACATAGC | |
| CTGA | |
| AOLFR206 sequences: | |
| MANRNNVTEFILLGLTENPKMQTIFVVFSVITTNAMIGNVLIVVTTASPSLRSPMTFLATLSFI | (SEQ ID NO:383) |
| DACYSSVNTPKTTDSLTENKTILFNGCMTQVFGEHFFRGVEVILLTVMAYDHYVAICKPLHYT | |
| TIMKQHVCSLLVGVSWVGQFLHATIQILFICQLPFCGPNVIDHFMCDLTTLNLACTTHTLGLF | |
| IAANSGFICLLNCLLLLVSCVVILTSLKTHSLEARHEALSTCVSIIITVVILSFIPCWTTMlTPATL | |
| PIDKAVAVFTTMITSMLNPLITTLRNAQMKNAIRKLCSRKAISSVK | |
| ATGGCGAATAGAAACAATGTGACAGAGTTTATTCTATGGGGCTTACAGAGTTTCCAAAA | (SEQ ID NO:384) |
| ATGCAGAAAATCATATTTGTTGTGTTTTCTGTCATCTACATCAACGCCATGATAGGAAATG | |
| TGCTCATTGTGGTCACCATCACTGCCAGCCCATCACTGAGATCCCCCATGTACTTTTTCCTG | |
| GCCTATCTCTCCTTTATTGATGGCTGCTATTCCTCTGTCTTACCCCTTGCTGATCACAGA | |
| TTCACTCTATGAAAACAAGACTATGTTATTCAATGGATGTATGACTCATGTCTTTGGAGAA | |
| CATTTTTTCAGAGGTGTTGAGGTCATCCTACTTACTGTAATGGCCTATGACCACTATGTGG | |
| CCATCTGCAAGCCCTTGCACTATACCACCATCATGAAGCAGCATGTTGTAGCCTGCTAGT | |
| GGGAGTGTCATGGGTAGGAGGCTTTTCTTCATGCAACCATACAGATCCTCTTCATCTGTCTT | |
| TTACCTTTCTGTGGTCCTAATGTCATAGATCACTTTATGTGTGATCTCTACACTTTGATCT | |
| TCTTGCCTGCACTAATACCCACACTCTAGGACTCTTCATTGCTGCCAACAGTGGGTTCATAT | |
| GCCTGTTAAACTGTCTCTPGCTCCTGGTCTCCTGCGTGGTCATACTGTACTCCTTAAATGACC | |
| CACAGCTTAGAGGCAAGGCATGAAGCCCTCTCTACCTGTGTCTCCCACATCACAGTTGTCA | |
| TCTTATCCTTTATACCCTGCATATTTGTGTAGATGAGACCTCCAGCTACTTTACCCATTGAT | |
| AAAGCAGTTGCTGTATTCTACACTATGATAAGTTCTATGTTTTACCCCTTTTCTACACCTT | |
| GAGGAATGCTCAAATGAAAAATGCCATTAGGAAATTGTGTAGTAGGGCTATTCTG | |
| TGTCAAATAA | |
| AOLFR207 sequences: | |
| MERTNDSTSTEFFLVGLSAHPKLQTVFFVLILWMTLMILLGNGVLISVIWDSHLHTPMTTFLCN | (SEQ ID NO:385) |
| LSFLDVCYTSSSVPLILASFLAVKKKVSFSGCMVQMFISFAMGATECMILGTMALDRYVAICYP | |
| LRYPVIMSKGATVAMAAGSWVTGLVDSVVQTAFAMQLPFCATFVCEILAILKLACADI | |
| SIMVISMTGSNLIVLVIPLLVISISTWIVATILRIPSTEGKHKAFSTCSTTLTTIWYGTIFFMYAKP | |
| ESKASVDSGNEDIIEALISLFTGVMTPMLNPLITSLTKTLCTMTSDGK | |
| ATGGAAAGGACCAACGATTCCACGTCGACAGAATTTTTCCTGGTAGGGCTTCTGCCCACC | (SEQ ID NO:386) |
| CAAAGCTCCAGACAGTTTTCTTCGTTCTAATTTTTGTGGATGTACCTGATGATCCTGCTTGGA | |
| AATGGAGTCGTTATCTCAGTTATCATCTTTGATTCTCACCTGCACACCCCCATGTATTTCTT | |
| CCTCTGTTTCTTTCCTTCCTCGACGTTGCTACACTGTTCCTCTGTCCCACTTTTCTTG | |
| CCAGCTTTGTGGCAGTAAAGAAAAAGGTTTCCTTCTCTGGGTGTATGGTGCATTTGTTAT | |
| TTCTTTTTGCCATGGGGGCCACGGAGTGCATGATCTTAGGCACGATGGCACTGGACCGCTAT | |
| GTGGCCATCTGCTACCCACTGAGATACCCTGTCATCATGAGCTGGGTGCCTATGTGGCCA | |
| TGGCAGCTGGGTCCTGGGTCACTGGGCTTGTGGACTCAGTAGTGCAGACAGCTTTTGCITT | |
| GCAGTTACCATTCTGTGCTAATAATGTCATTAAACATTTTGTCTGTGATTTCTGGCTATCT | |
| TGAAACTGGCCTGTGCTGATATTTCAATCAATGTGATTAGTATGACAGGGTCGITTCTGAT | |
| TGTTCTGGTTATTCCATTGTTAGTAATTTCCATCTCTTACATATTTATTGTTGCCACTATTCT | |
| GAGGATTCCTTCCACTGAAGGAATAACATAAGGCCTTCTCCACCTGCTCAGCCCACCTGACA | |
| GTGGTGATTATATTCTATGGAACCATCTTCTTCATGTACGGTGCCTGAGTGTATGCCT | |
| CTGTTGATTCAGGTAATGAAGACATCATTGAGGCCCTCATCTCCGTTTTCTATGGAGTGAT | |
| GACTCCCATGCTPAATCCTCTCATGTATAGTCTGCGACTGGATGTATTGGCTGCTGTC | |
| AAAAACATACTGTGTAGGAAAAACTTTTCTGATGGAATTGA | |
| AOLFR208 sequences: | |
| MFPANWTSVKVFFFLGFFHTPKVQVIIFAVCLLMTLITLLGMFLISITILDSHLHTPMTFLSNL | (SEQ ID NO:387) |
| SFLDIWTSSSALSPMLANFVSGRNTISFSGCATQMTLSLAMGSTECVLLPMRTTMCT | |
| LRYPVIMRTCVQLTAGSWMTGCLTAMVEMMSVLPLSLCGNSITTCEILAThTVCTTS | |
| LVQLIMLVISVLLLPMPMLLICISTAFILASILRISSVEGRSKTSTCTTLLFTGTTSMTT | |
| LKPSAVDSQEIDKFMALVYAGQTPMLNPIIYSLRNKEVKVALKKLLIRNHFNTAFISILK | |
| ATGTTCCCGGCAAATTGGACATCTGTAAAAGTATTTTCTTCCTGGGATTTTTTCAGTACCC | (SEQ ID NO:388) |
| CAAAGTTCAGGTCATCATATTTGCGGTGTGCTTGCTGATGTACCTGATCACCTTGCTGGGC | |
| AACATTTTTCTGATCTCCATCACCATTCTAGATTCCCACCTGCACACCCCTATGTACCTCTT | |
| CCTCAGCAATCTCTCCTTTCTGGACATCTGGTACTCCTCTTCTGCCCTCTCTCCTTGCTGG | |
| CAAACTTTGTTTCAGGGAGAAACACTATTTCATTCTCAGGGTGCGCCACTCAGATGTACCT | |
| CTCCCTTGCCATGGGCTCCACTGAGTGTGTGCTCCTGCCCATGATGGCATATGACCGGTAT | |
| GTGGCCATCTGCTCCCCCTGAGATACCCTGTCATCATGTTAGGAGTCCTGTGTGCAGA | |
| TTGCAGCTGGCTCCTGGATGACAGGCTGTCTCACTGCCATGGTGGMATGATGTCTGTGCT | |
| GCCACTGTCTCTCTGTGGTAATAGCATCATCAATCATTTCACTTGTGATTTCTGGCCATCT | |
| TGAAATTGGTTTGTGTGGACACCTCCCTGGTGCAGTTAATCATGCTGGTGATCAGTGTACT | |
| TCTTCTCCCCATGCCAATGCTACTCATTTGTATCTCTTATGCATTTATCGTCGCCAGTATCC | |
| TGAGAATCAGCTCAGTGGAAGGTCGAAGTAAAGCCTTTTCAACGTGCACAGCCCACCTGA | |
| TGGTGGTAGTTTTGTTCTATGGGACGGCTCTCTCCATGCACCTGTTGCCCTGCGCTGTAGA | |
| TTCACAGGAAATAGACAAATTTATGGCTTTGGTGTATGCCGGACTACCCCCATGTTGTT | |
| CCTATCATCTATAGTCTACGGAACAAAGAGGTGAAAGTGGCCTTGAAAAAATGCTGATTA | |
| GAAATCATTTTAATACTGCCTTCATTTCCATCCTCAAATT | |
| AOLFR209 sequences: | |
| MDKINQTFVREFILLGLSGTPKLEIIFFALILVMTVVILIGNGVLIIASILDSLHMPMTTFLGNLS | (SEQ ID NO:389) |
| FLDICTTTSSIPSTLVSLISKKRNISFSGCAVQMFFGFAMGSTECFLLGTDRTAICNPLRY | |
| PIIMNKVVTVLLTSVS WLSGGIINSTVQTSLAMRWPFCGNMP4HFLCEILAVLKLACSDISVNIV | |
| TLAVSNIAFLVLPLLVIFFSTMFILTTILRTNSATGRTSTCSAIILTTIIFTGTWFMYAKPKS | |
| QDLLGKDNLQATEGLVSMFTGVVTPMLNPIITSLRKDVKAAIKTLLSRKAINQ | |
| ATGGACAAGATAAACCAGACATTTGTGAGAGAATTCATTCTTCTGGGACTCTCTGGTTACC | (SEQ ID NO:390) |
| CGTACTTGAGATCATTTTCTTTGCTCTGATTCTAGTTATGTACGTAGTGATCTTTTGGC | |
| AATGGTGTTCTGATCATAGCAAGCATCTTGGATTCTCGTCTTCACATGCCCATGTACTTCTT | |
| CCTGGGCAACCTCTCTTTCCTGGATATCTGCTATACAACCTCCTCCATTCCCTCTTCACTGG | |
| TGAGCTTAATCTCAAAGAAAAGAAACATTTCGTTCTCTGGATGTGCAGTGCAGATGTTCTT | |
| TGGGTTTGCAATGGGGTCAACAGAATGTTTCCTCCTTGGCATGATGGCATTTGATCGTTAT | |
| GTGGCCATCTGTAACCCTCTGAGATACCCCATCATCATGAACTGGTGGTGTATGTACTGC | |
| TGACTTCTGTATCATGGCTTTCTGGTGGAATCAATTCAACTGTGCTACATCACTTGCCAT | |
| GCGATGGCCTPTCTGTGGGAACAATATTATTAATCATTTCTTATGCGAGATCTTAGCTGTCC | |
| TAAAATTAGCTTGTTCTGATATATCTGTCAATATTGTTACCCTAGCAGTGTCTTATATTGCT | |
| TTCCTAGTTCTTCCTCTGCTCGTGATTTTTTTCTCCTATATGTTCATCCTCTACACCATCTTG | |
| CGAACGAACTCGGCCACAGGAAGACACAAGGCATTTTCTACATGCTCAGCTCACCTGATG | |
| TGGTGATCATATTTTATGGTACCATCTTCTTTATGTATGCAAAACCTTGTCCCAGGACCTC | |
| CTTGGGAAAGACAACTTGCAAGCTACAGAGGGGCTTGTTCCATGTTTTATGGGGTTGTGA | |
| CCCCCATGTTAAACCCCATAATCTATAGCTTGAGAAATATGATGTTGCTGCTATTT | |
| ATATTTGCTGAGCAGGAAAGCTATTAACCAGTAA | |
| AOLFR210 sequences: | |
| MMGRRNDTNVADFILTGLSDSEEVQMALFMLFLLIYLITMLGNVGMLLIIRLDLQLHTPMYFFL | (SEQ ID NO:391) |
| THLSFIDLSTSTVVTPKTLANLLTSNTSFTGCFAQMFCFVFLGTAECTLLSSTTTRTTICSP | |
| LHYTVIMPKRLCLALITGPTVIGFMDSFVNVVSMSRLHFCDSNIIHHFFCDTSPILALSCTDTDN | |
| TEMLIFIIAGSTLMVSLITISASTVSILSTILKTNSTSGKQKAFSTCVSHLLGVTIFTGTMIFTYLKP | |
| RKSTSLGRDQVAPVFTTIVIPMLNPLITSLRINREVIKNALIRVMQRRQDSR | |
| ATGATGGGTAGAAGGAATGACACAAATGTGGCTGACTTCATCCTTTACGGGACTGTCAGAC | (SEQ ID NO:392) |
| TCTGAAGAGGTCCAGATGGCTCTGTTTATGCTATTTCTCGTCATATACCTAATTACTATGCT | |
| GGGGAATGTGGGGATGCTATTGATAATCCGCCTGGACCTCCAGCTTCACACTCCCATGTAT | |
| TTTTTCCTTACTCACCTGTCATTTATTGACCTCAGTTACTCAACTGTCGTCACACCTTTTC | |
| CTTAGCGAACTTACTGACTTCCAACTATATTTCCTTCACGGGCTGCTTTGCCCAGATGTTCT | |
| GTTTTGTCTTCTTGGGTACTGCTGAATGTTATCTTCTCTCCTCAATGGCCTATGATCGCTAT | |
| GCAGCGATCTGCAGTCCTCTACACTACACAGTTATTATGCCCAAAAGGCTCTGCCTCGCTC | |
| TCATCACTGGGCCTTATGTGATTGGCTTTATGGACTCCTTTGTCAATGTGGTTTCCATGAGC | |
| AGATTGCATTTCTGTGACTCAAACATAATTCATCACTTTTTCTGTGACACTTCCCCTTTTT | |
| AGCTCTGTCCTGGACTGACACAGACAACACTGAAATGCTGATATTCATTATGGCTGGTTCC | |
| ACCCTGATGGTGTCCCTTATCACAATATCTGCATCCTATGTGTCCATTCTCTCTACCATCCT | |
| GAAAATTAATTCCACTTCAGGAAAGCAGAAAGCTTTCTCTACTTGCGTCTCTCATCTCTTG | |
| GGAGTCACCATCTTCTATGGAACTATGATTTTTACTTACTTAAAGCCAAGAAAGTCTTATT | |
| CCTTGGGAAGAGATCAAGTGGCTCCTGTGTTTfATACTATTGTGATTCCCATGCTGITTCC | |
| ACTCATTTATAGTCTTAGAAACAGAGAAGTGAAAAATGCTCTCATTAGAGTCATGCAGAG | |
| AAGACAGGACTCCAGGTAG | |
| AOLFR211 sequences: | |
| MMGRRTTNTNVADFILMGLTLSEEIQMALFMLFLLITLITMLGNVGMILIIRLDLQLHTPMYFFL | (SEQ ID NO:393) |
| THLSFIDLSTSTVVTPKTLANLLTSNTSFTGCFAQMFFFAFLGTAECTLLSSMAHDRTAAICSP | |
| LHYTVIMSKRLCLALITGPTVIGFIDSFVNVVSMSRLHFTDSNVIHHFFCDTSPILALSCTDTYNT | |
| EILIFIIVGSTLMVSLFTISASTVFILFTILKTNSTSGKQKAFSTCVSHLLGVTIFTSTLIFTTLKTPRK | |
| SYSLGRDQVASVFTTIVIPVLNPLITSLRNKEVKNAVIRVMQRRQDSR | |
| ATGATGGGTAGAAGGAATAACACAAATGTGGCTGACTTCATCCTTATGGGACTGACACTTT | (SEQ ID NO:394) |
| CTGAAGAGATCCAGATGGCTCTGTTTATGCTATTTCTCCTGATATACCTTTTACTATGCTG | |
| GGGAATGTGGGGATGATKTTGATAATCCGCCTGGACCTCCAGCTTCACACTCCCATGTATT | |
| TTTTCCTTACTCACCTGTCATTTATTGACCTCAGTTACTCTCTGTCGTCACACCTTTCC | |
| TTAGCGAACTTACTGACTTCCAACTATATTTCCTTTACGGGCTGCTTTGCCCAGATGTTCTT | |
| TTTTGCCTTCTTGGGTACTGCTGAATGTTACCTTCTCTCCTCTTGGCCCATGATCGCTATG | |
| CAGCGATCTGCAGTCCTCTACACTACACAGTTATTATGTCCAATGGCTCTGCCTCGCTCT | |
| CATCACTGGGCCTTATGTGATTGGCTTTATAGACTCCTTTGTCTCGTGGTTCCATGAGCA | |
| GATTGCATTTCTACGACTCAAACGTAATTCATCACTTTTTCTGTGACACTTCCCCAATTTTA | |
| GCTCTGTCCTGCACTGATACATACAACAGCGAAATCCTGATATTCATTATTGTGGTTCCAC | |
| CCTGATGGTGTCCCTTTTCACAATATCTGCATCCTATGTGTTCATTCTCTTTACCATCCTGA | |
| AAATTAATTCCACTTCAGGAAAGCAGAAAGCTTTCTCTACTTGCGTCTCTCATCTCTTGGG | |
| AGTCACCATCTTTTATAGCACTCTGATTTTTACTTATTTAAAACCAAGAAAGTCTTATTCCT | |
| TGGGAAGAGATCAAGTGGCTTCTGTTTTTTATACTATTGTGATTCCCGTGCTGAATCCACT | |
| CATTTATAGTCTTAGAAACAAAGAGGTGAAAAATGCTGTCATCAGAGTCATGCAGAGTG | |
| ACAGGACTCCAGGTAA | |
| AOLFR212 sequences: | |
| MAGNNFTEVTVFILSGFANHPELQVSLFLMFLFITLFTVLGNLGLITLITTIDSQLHTPMTFLSN | (SEQ ID NO:395) |
| LAFIDWTSSTVTPKALVNFQSNRRSISFVGCFVQMTTFVGLVCCECFLLGSMAYNRYIAICNPL | |
| LYSVVMSQKVSNWLGVMPTVIGFTSSLISVWVISSLAFCDSSINHFFCDTTALLALSCTTFGT | |
| EMVSFVLAGFTLLSSLLIITVTTIIISAILRQSAAGRQKTFSTCASHLTVTIFTGSLWTTLQPD | |
| NTSSLTQAQVASVFTTIVIPMLNPLITSLRNKDVKNALLRVIHRKLFP | |
| ATGGCTGGCAACTTTCACTGAGGTTACCGTCTTCATCCTCTCTGGATTTGCKTTCACC | (SEQ ID NO:396) |
| CTGAATTACAAGTCAGTCTTTTCTTGATGTTTCTCTTCATTTATCTATCACTGTTTTGGGA | |
| AACCTGGGACTGATCACGTTAATCAGAATGGATTCTCAGCTTCACACCCCTATGTACTTT | |
| TCCTGAGCAATTTAGCATTTATTGACATATTTTACTCCTCTACTGTAACACCTTTGGCATTG | |
| GTGAATTTCCAATCCAATCGGAGATCCATCTCCTTTGTTTGGCTGCTTTGTTGTATGTACTT | |
| TTTTGTTGGATTGGTGTGTTGTGAGTGTTTCCTTCTGGGATCAATGGCCTACTTCGCTACA | |
| TAGCAATCTGCAATCCCTTACTGTATTCAGTAGTCATGTCCCAAATTGTGTCCTTCTGGCT | |
| GGGAGTAATGCCATATGTGATAGGCTTCACAAGCTCGCTGATATCTGTCTGGGTGATTTGC | |
| AGTTTGGCGTTCTGTGATTCCAGCATCAATCATTTTTTTTGTGACACCACAGCTCTTTAGC | |
| ACTCTCCTGTGTAGATACATTCGGCACAGAAATGGTGAGCTTTGTCTTAGCTGGATTCACT | |
| CTTCTTAGCTCTCTCCTTATCATCACAGTCACTTATATCATCATCATCTCAGCCATCCTGAG | |
| GATCCAGTCAGCAGCAGGCAGGCAGAAGGCCTTCTCCACCTGCGCATCCCACCTCATGGCT | |
| GTTCTATCTTTATGGGTCTCTGATTTTCACCTATTTGCTCCTGATTCACATCATCGCT | |
| GACCCAGGCGCAGGTGGCATCTGTATTCTATACGATTGTCATTCCCATGCTGTTCCACTC | |
| ATCTACAGTCTGAGGAACAAAGATGTGAAAAATGCTCTTCTGAGAGTCATACATAGKT | |
| CTTTTTCCATGA | |
| AOLFR213 sequences: | |
| MNSLGKLVSMILSAHVFCTSKENCFGCTHSIPALGADPPGGMGLGNESSLMDFILLGFSDHPRL | (SEQ ID NO:397) |
| EAVLFVFVLFFTLLTLVGNFTIIIISTLDPPLHTPMTTFLSNLSLLDICFTTSLAPQTLTLQRPKK | |
| TITYGGCVAQLTSLALGSTECILLADMALDRTAVCKPLHTVVIMNPRLCQQLASISWLSGLA | |
| SSLIHATFTLQLPLCGNIIRLDHFICEVPALLKLACVDTTVNELVLFVVSVLFVVIPPALISISTGFI | |
| TQAVLRIKSVEARHKMSTCSSHLTVVIIFTGTIITVTLQPSDSTAQDQGKEISLFTTMVTPTLNP | |
| IITTLRNKDMKEALRKLLSGKL | |
| ATGAATAGTTTGGGATGTTGGTCTGCATGATCCTCTCAGCTCATGTGTTCTGTATTCTT | |
| ATTTAATTGTTTTGGATGTACCCATTCCATTCCTGCCTTAGGTGCGGATCCCCCTGGAGGG | |
| ATGGGATTGGGCAATGAGAGTTCCCTAATGGATTTCATCCTTCTAGGCTTCTCAGACCACC | |
| CTCGTCTGGAGGCTGTTCTCTTTGTATTTGTCCTTTTCTTCTACCTCCTGACCCTTGTGGGA | |
| AACTTCACCATAATCATCATCTCATATCTGGATCCCCCTCTTITCATACCCCAATGTACTTTTT | |
| TCTCAGCAACCTCTCTTTACTGGACATCTGCTTCACTACTAGCCTTGCTCCTCAGACCTTAG | |
| TTAACTTGCAAGACCTAGAGACGATCACTTACGGTGGTGTGTGGCGCTCTCTATAT | |
| TTCTCTGGCACTGGGCTCCACTGAATGTATCCTCTTGGCTGACATGGCCTGGATCGGTAC | |
| ATTGCTGTCTGCAAACCCCTCCACTATGTAGTCATCATGAACCCACGGGTTTGCCTCAGC | |
| TGGCATCTATCTCCTGGCTCAGTGGTTTGGCTAGTTCCCTTTTCCATGCTCTTTTACCTTG | |
| CAATGCCTCTCTGTGGCAACCATAGGCTGGACCATTTTATTTGCGAAGTACCAGCTCTTCT | |
| CTGTTGGCTTGTGTGGACACCACTGTCTTGAATTGGTGCTTTTTGTTGTAGTGTCTGT | |
| TGTTGTCATTCCACCAGCACTCATCTCCATCTCCTATGGCTTCATTCTCTGCTGTGCTG | |
| AGGATCAAATCAGTAGAGGCAAGGCATAAAGCCTTCAGCACCTGCTCGTCCCACCTTACAG | |
| TGGTGATTATATTCTATGGCACCATAATCTACGTGTACCTGCTCCTAGTGACAGCTATGC | |
| CGAGGACCAAGGGAAGTTTATCTCCCTCTTCTACACCATGGTGACCCCCACTTTATTCCT | |
| ATCATCTATACTTTAAGGAACAAGGATATGAAAGAGGCTCTGAGGATCTTCTCTCGGGA | |
| AAATTGTGA | |
| AOLFR214 sequences: | |
| MDKSNSSVVSEFVLLGLCSSQKLQFYFCFFSVLYTVIVLGNLLIILTVTSDTSLHSPMYFLLGN | (SEQ ID NO:399) |
| LSFVDICQASFATPKMIADFLSAIIETISFSGCIAQIFFIHLFTGGEMVLLVSTTRTTAICTLT | |
| TVVIMSRRTCTVLVMISWAVSLVHTLSQLSFTVNLPFCGPNVTSFFCDLPRVTTACLDSTIE | |
| ILlVVNSGILSLSTFSLLVSSTHLVTVWLKSSAAMAKAFSTLASHIAVVILFFGPCIFITWPFTIS | |
| PLDKFLAIFTTVFTPVLNPIITTLRNRDMKAAVRTHTLRITISEMSLVTTSFH | |
| ATGGATAAGTCCAATTCTTCAGTGGTGTCTGAATTTGTACTGTTGGGACTCTGTAGTTCTC | (SEQ ID NO:400) |
| AAAAACTCCAGCTTTTCTATTTTTGTTTCTTCTCTGTGTTGTATACAGTCATTGTGCTGGGA | |
| AATCTTCTCATTATCCTCACAGTGACTTCTGATACCAGCCTGCACTCCCCTATGTACTTCT | |
| CTTGGGAAACCTTTCCTTTGTTGACATTGTCAGGCTTCTTTGCTACCCCTTTTTTGATTG | |
| CAGATTTTCTGAGTGCACACGAGACCAIATCTTTCAGTGGCTGCATAGCCCTATTTTCTT | |
| ATTCACCTTTTTACTGGAGGGGAGATGGTGCTACTTGTTTCGATGGCCTATGACAGGTATG | |
| TAGCCATATGCAAACCCTTATACTATGTGGTCATCATGAGCCGTTGGACATGCAGTGTCTT | |
| GGTAATGATCTCCTGGGCTGTGAGCTTGGTGCACACATTTGCGAGTTATCATTTACTGTG | |
| AACCTGCCTTTTTGTGGACCTAATGTAGTAGACAGCTTTTTTTTGTGATCTCCTCGAGTCAC | |
| CAAACTTGCCTGCCTGGACTCTTACATCATTGAAATACTTTTGTGGTCTTAGTGGTTT | |
| CTTTCCCTAAGCACTTTCTCTCTCTTGGTCAGCTCCTACATCATTATTCTGTTACAGTTTG | |
| GCTCAAGTCTTCAGCTGCAATGGCAAAGGCATTTTCTACGCTGGCTTCCCATATTGCAGTA | |
| GTAATATTATTCTTTGGACCTTGCATCTTCATCTATGTGTGGCCCTTTACCATCTCTCCTTT | |
| GGATAAATTTCTTGCCATATTTTACACTGTTTTCACCCCCGTCCTATTTCCCCATTATTTATA | |
| CACTAAGGAATAGGGATATGAAGGCTGCCGTAAGGAAAATTGTGITCCATTACCTGAGGC | |
| CAAGGAGAATTTCTGAAATGTCACTAGTAGTGAGAACTTCCTTTCATTT | |
| AOLFR215 sequences: | |
| MAHTNESMVSEFVLLGLSNSWGLQLFFFAIFSIVTVTSVLGNVLIIVIISFDSHLNSPMYFLLSNL | (SEQ ID NO:401) |
| SFIDICQSNTATPKMLVDFFIERKTISFEGCMAQIFVLHSFVGSEMMLLVAMATDTCKPLH | |
| YSTIIVTNRRLCVIFVSISWAVGVLHSVSHLAFTVDLPFCGPNEVDSFFCDLPLVIELACMDYEM | |
| EIMTLTNSGLISLSCFLALIISTTIILIGVRCRSSSGSSKALSTLTAHITVVILFFGPCIITWPFSRL | |
| PVDKFLSVFTTVCTPLLNPIITSLRNEDVKAAMWKLRNHHVNSWKN | |
| ATGGCTCACACAAATGAATCGATGGTGTCTGAGTTTGTACTTTTGGGACTCTCTAATTCCT | (SEQ ID NO:402) |
| GGGGACTTCAACTTTTCTTTTTCGCCATCTTCTCTATAGTCTATGTGACATCAGTGCTAGGC | |
| AATGTCTTAATTATTGTCATTATTTCTTTTGACTCCCATTTTGAACTCTCCTATGTACTTCTG | |
| CTCAGTAATCTTTCTTTCATTGATATCTGTCAGTCTAACTTTGCCACCCCCTTGATGCTTGT | |
| AGACTTTTTTATTGAGCGCAAGACTATCTCCTTTGAGGGTTGCATGGCCCAGATATTCGTT | |
| CTTCACAGTTTTGTTGGGAGTGAGATGATGTTGCTTGTAGCTATGGCATATGACAGATTTA | |
| TAGCCATATGTAAGCCTCTGCACTACAGTACAATTATGAACCGGAGGCTCTGTGTTTTTTT | |
| TGTGTCTATTTCCTGGGCGGTGGGCGTTGTTCATTCTGTGAGCCACTTGGCTTTTACAGTGG | |
| ACCTGCCATTCTGTGGTCCCAATGAGGTGGATAGCTTCTTTTGTGACCTTCCCTGGTGATA | |
| GAGCTGGCTTGCATGGATACATATGAAATGGATTTATGACCCTTCGITCAGTGGCCTG | |
| ATATCATTGAGCTGTTTCCTGGCTTTAATTATTTCCTACACCATCATTTTGATCGGTGTCCG | |
| ATGCAGGTCCTCCAGTGGGTCATCTAAGGCTCTTTCTACATTAACTGCCCACATCACAGTG | |
| GTCATTCTTTTCTTCGGGCCTTGCATTTATTTCTATATATGGCCTTTTAGCAGACTTCCTGT | |
| GGACAAATTTCTTTTCTGTGTTCTACACTGTTTGTACTCCCTTGTTTGAACCCCATCATCTACT | |
| CTTTGAGGAATGAAGATGTTAAAGCAGCCATGTGGAAGCTGAGAAACCATCATGTGAACT | |
| CCTGGAAAAACTAG | |
| AOLFR216 sequences: | |
| MDVGNKSTMSEFVLLGLSNSWELQMFFFMVFSLLTVATMVGNSLIVITVIVDPHLHSPMTTLL | (SEQ ID NO:403) |
| TNLSIIDMSLASFATPKMITDTLTGHKTISFDGCLTQIFFLHLFTGTEIILLMAMSFDRTAICKPL | |
| HYASVISPQVCVALVVASWIMGVMHSMSQVIFATTLPFCGPTEVDSFFCDLPVVFQLACVDTY | |
| VLGLFMISTSGIIALSCFIVLFNSTVIVLVTVKHHSSRGSSKALSTCTAHFIVVFLFFGPCIFITMW | |
| PLSSFLTDTLSVFTTIFTPTLNPIITTLRNQEVTAMRKLKNRFLNFNKAMPS | |
| ATGGATGTGGGCAATAAGTCTACCATGTCTGAATTTGTTTTGCTGGGGCTCTCTAATTCCT | (SEQ ID NO:404) |
| GGGAACTACAGATGTTTTTCTTTATGGTGTTTTCATTGCTTTATGTGGCAACAATGGTGGG | |
| TAACAGCCTCATAGTCATCACAGTTATAGTGGACCCTCACCTACACTCTCCTATGTATTTCC | |
| TGCTTACCAATCTTTCAATCATTGATATGTCTCTTGCTTCTTTCGCCACCCCAAAGATGATT | |
| ACAGATTACCTAACAGGTCACAAAACCATCTCTTTTGATGGCTGCCTTACCCAGATATTCT | |
| TTCTCCACCTTTTCACTGGAACTGAGATCATCTTACTCATGGCCATGTCCTTTGATAGGTAT | |
| ATTGCAATATGCAAGCCCCTGCACTATGCTTCTGTCATTAGTCCCCAGGTGTGTGTTGCTCT | |
| CGTGGTGGCTTCCTGGATTATGGGAGTTATGCATTCAATGAGTCAGGTCATATTTGCCCTC | |
| ACGTTACGATTCTGTGGTCCCTATGAGGTAGACAGCTTTTTCTGTGACCTTCCTGTGGTGTT | |
| CCAGTTGGCTTGTGTGGATACTTATGTTCTGGGCCTCTTTATGATCTCAACAAGTGGCATA | |
| ATTGCGTTGTCCTGTTTTATTGTTTTATTTAATTCATATGTTATTGTCCTGGTTACTGTGAA | |
| GCATCATTCTTCCAGAGGATCATCTAAGGCCCTTTCTACTTGTACAGCTCATTTCATTGTTTG | |
| TCTTCTTGTTCTTTGGGCCATGCATCTTCATCTACATGTGGCCACTAAGCAGCTTTCTCACA | |
| GACAAGATTCTGTCTGTGTTTTATACCATCTTTACTCCCACTCTGAACCCAATAATCTATAC | |
| TTTGAGGAATCAAGAAGTAAAGATAGCCATGAGGAAACTGAAAAATAGGTTTCTAAATTT | |
| TAATAAGGCAATGCCTTCATAG | |
| AOLFR217 sequences: | |
| MLESFQKSEQMAWSNQSAVTEFILRGLSSSLELQIFTTLFFSIVTAATVLGNLLIVVTIASEPHLH | (SEQ ID NO:405) |
| SPTYFLLGNLSFIDMSLASFATPKMIADFLRIEIIKAISFEGCMTQMFFLHLLGGAEIVLLISMSFD | |
| RYVAICKPLHTLTIMSRRMCVGLVILSWIVGIFHALSQLAETVNLPFCGPNEVDSFFCDLPLVIK | |
| LACVDTTLGVFMISTSGMIALVCFILLVISTTIILVTVRQRSSGGSSKALSTCSAIIFTVVTLFFGP | |
| CTFIYVWPFTNFPIDKVLSVFTTITTPLLNPVITTVRNKDVKTSMiRKLSSHIFKSRKTDHTP | |
| ATGCTAGAGTCCTTCCAGAAATCAGAGCAAATGGCCTGGAGCAATCAGTCTGCGGTAACC | (SEQ ID NO:406) |
| GAATTCATACTACGGGGTCTGTCCAGTTCTTTAGAACTCCAGATTTTCTACTTGCTGTTTTT | |
| CTCGATAGTCTATGCAGCCACTGTGCTGGGGAACCTTCTTATTGTGGTCACCATTGCATCA | |
| GAGCCACACCTTCATTCCCCTACGTACTTTCTGCTGGGCAATCTCTCGTTCATTGACATGTC | |
| CCTGGCCTCATTTGCCACCCCCAAAATGATTGCAGACTTCCTTAGAGAACACAAAGGCATC | |
| TCTTTTGAAGGCTGCATGACCCAGATGTTCTTCCTACATCTCTTAGGGGGTGCTGAGATTG | |
| TACTGCTGATCTCCATGTCCTTTGATAGGTACGTGGCTATCTGTAAGCCTCTACATTACCTA | |
| ACAATCATGAGCCGAAGAATGTGTGTTGGGCTTGTGATACTTTGCTGGATTGTCGGCATCT | |
| TCCATGCTCTGAGTCAGTTAGCATTTACAGTGAATCTGCCCTTCTGTGGACCCAATGAAGT | |
| AGACAGTTTCTTTTGTGACGTCCCTTTGGTGATTAAACTTGCTTGTGTCGACACATATATTC | |
| TGGGGGTGTTCATGATCTCAACCAGTGGCATGATTGCCCTGGTGTGCTTCATCCTCTTGGT | |
| GATCTCTTACACTATCATCCTGGTCACCGTTCGGCAGCGTTCCTCTGGTGGATCCTCCAAA | |
| GCCCTCTCCACGTGCAGTGCCCACTTTACTGTTGTGACCCTTTTCTTTGGCCCATGCACTTT | |
| CATTTATGTGTGGCCTTTCACAAATTTTCCCAATAGACAAAGTACTCTCAGTATTTTATACCA | |
| TATACACTCCCCTCTTGAATCGAGTGATCTATACCGTTAGGAATAAAGATGTCAAGTATTC | |
| CATGAGGAAAGTAAGCAGCCATATCTTTAAATCTAGGAAGACTGATCATTACTCCTTAA | |
| AOLIFR218 sequences: | |
| METANTTKVTEFVLTGLSQTREVQLVLFVIFLSFTLFILPGNILIICTIRLDPHLTSPMTTLLANLA | (SEQ ID NO:407) |
| LLDIWTSSITAjPKMLIDFFVERTISFGGCIAQLFFLHEVGASEMFLLIVMATDRTAAICRPLHYA | |
| TIMNRRLCCILVALSWMGGFIHSIIQVALIVRLPFCGPNELDSTTCDITQVVRIACANTFPEELVM | |
| ICSSGLISVVCFIALLMSTAFLLALLKTHSGSDENTNRAMSTCTSHITIVVLMFGPSITTARPFD | |
| SFSLDKVVSVFHTVWPLLNPIITTLRNKEVKAAMRKVVTKTLCEEK | |
| ATGGAAACTGCAAATTACACCAAGGTGACAGAATTTGTTCTCACTGGCCTATCCCAGACTC | (SEQ ID NO:408) |
| GGGAGGTCCAACTAGTCCTATTTGTTATATTTCTATCCTTCTATTTGTTCATCCTACCAGGA | |
| AATATCCTTATCATTTGCACCATCAGGCTAGACCCTCATCTGACTTCTCCTATGTATTTCCT | |
| GTTGGCTAATGTGGCCCTCCTTGATATTTGGTACTCTTCCATTACAGCCCCTAAAATGCTCA | |
| TAGACTTCTTTGTGGAGAGGAAGATAATTTCCTTTGGTGGATGCATTGCACAGCTCTTCTT | |
| CTTACACTTTGTTTGGGGCTTCGGAGATGTTCTTGCTCATAGTGATGGCCTATGACCGCTAT | |
| GCTGCTATCTGCCGACCCCTCCACTATGCTACCATCATGAATCGACGTCTCTGCTGTATCCT | |
| GGTGGCTCTCTCCTGGATGGGGGGCTTCATTCATTCTATAATACAGGTGGCTCTCATTGTT | |
| CGACTTCCTTTCTGTGGGCCCAATGAGTTAGACAGTTACTTCTGTGACATCACACAGGTTG | |
| TCCGGATTGCCTGTGCCAACACCTTCCCAGAGGAGTTAGTGATGATCTGTAGTAGTGGTCT | |
| GATCTCTGTGGTGTGTTTCATTGCTCTGTTAATGTCCTATGCCTTCCTTCTGGCCTTGCTCA | |
| AGAAACATTCAGGCTCAGATGAGAATACCAACAGGGCCATGTCCACCTGCTATTCCCACAT | |
| TACCATTGTGGTGCTAATGTTTGGGCCATCCATCTACATTTATGCTCGCCCATTTGACTCAT | |
| TTTCCGTAGATAAAGTGGTGTCTGTGTTTCATACTGTAATATTCCCTTTTACTTAATCCGATT | |
| ATTTACACATTGAGAAACAAGGAAGTAAAGGCAGCCATGAGGAAGGTGGTCACCAAATAT | |
| ATTTTGTGTGAAGAGAAGTGA. | |
| AOLFR219 sequences: | |
| MLTSLTDLCFSPIQVAEIKSLPKSMNETNHSRVTEFVLLGLSSSRELQPFLFLTFSLLTLAILLGNF | (SEQ ID NO:409) |
| LIILTVTSDSRLHTPMTTLLANLSFIDVCVASFATPKMIADFLVERKTISFDACLAQIFFVHLFTGS | |
| EMVLLVSMATDRTVAICKPLHTMTVMSRRVCVVLVLISWIFVGFIHTTSQLALFTVNLPFCGPN | |
| KVDSFFCDLPLVTKLACIDTTVVSLLIVADSGFLSLSSFLLLVVSTTVILVTVRNRSSASMAKAR | |
| STLTAHITVVTLFFGPCIFITVWPFSSTSVDKVLAVFTTIFTLILTTPVITTLRNKEVKAAMSKLKS | |
| RYLKPSQVSVVIRNVLFLETK. | |
| ATGCTCACTTCATTAACTGATCTCTGTTTCTCTGCTATTCAGGTAGCTGAAATTAAGTCCCT | (SEQ ID NO:410) |
| TCCAAAATGGATGAATGAGACAAATCATTCTCGGGTGACAGAATTTGTGTTGCTGGGACTG | |
| TCTAGTTCAAGGGAGCTGCAACCTTTCTTGTTTCTTACATTTTCACTACTTTATCTAGCAAT | |
| TCTGTTGGGCAACTTTCTCATCATCCTCACTGTGACCTCAGATTCCCGGCTTGACACCCCCA | |
| TGTACTTTCTGCTTGCAAACCTGTCATTTATAGACGTATGTGTTGCGTCTTTTGGTACCCCT | |
| AAAATGATTGCAGACTTTTCTGGTTGAGCGCAAGACTATTTCTTTTGATGCCTGCCTGGCCC | |
| AGATTTTCTTTGTTCATCTCTTTCACTGGCAGTGAAATGGTGCTCCTAGTTTCCATGGCCTAT | |
| GACCGTTATGTTGCTATATGCAAACGTCTCCACTACATGACAGTCATGAGCCGTCGTGTAT | |
| GTGTTGTGCTCGTCCTCATTTCATGGTTTGTGGGCTTCATCCATACTACCAGCCAGTTGGCA | |
| TTCACTGTTAATCTGCCATTTTGTGGTCCTAATAAGGTAGACAGTTTTTTCTGTGACCTTCC | |
| TGTAGTGACCAAGTTAGGCTGCATAGACACTTATGTTGTCAGCTTACTAATAGTTGCAGAT | |
| AGTGGCTTTCTTTCTCTGAGTTCCTTTCTCCTCTTGGTTGTCTCCTACACTGTAATACTTGTT | |
| ACAGTTAGGAATCGCTCCTCTGCAAGCATGGCGAAGGCCCGCTCCACATTGACTGCTCACA | |
| TCACTGTGGTGACITTATTCTTTGGACCATGCATTTTCATCTATGTGTGGCCCTTCAGCAGT | |
| TACTCAGTTGACAAAGTCCTTGCTGTATTCTACAGCATCTTCACGCTTATTTTAAACCCTGT | |
| AATCTACACGCTAAGAAACAAAGAAGTGAAGGCAGCTATGTCAAAACTGAAGAGTCGGTA | |
| TCTGAAGCCTAGTCAGGTTTCTGTAGTCATAAGAAATGTTCTTTTCCTAGAAACAAAGTAA. | |
| AOLFR22 sequences: | |
| MKQTSVGNQHSNTRSLLFPFLCSQMTQLTASGNQTMVTEFLFSMFPHAHRGGLLFFIPLLLIYG | (SEQ ID NO:411) |
| FILTGNLIMFIVIQVGMALIITPLTTFISVLSFLEICTTTTTIPKMLSCLISEQKSISVAGCLLQMYFF | |
| HSLGITESCVLTAMAIDRTAICNPLRTPTIMIPKLCIQLTVGSCFCGFLLVLPEIAWISTLPFCGS | |
| NQIHQIFCDFTPVLSLACTDTFLVVIVDAIIIAAEIVASFLVIALSTRIIIVILGMHSAEGHHKALFST | |
| CAALHLAVFLLFFGSVAVMTLRIFSATTSVEWDTAIAVTFVILAPFFNPIITSLKNKDMIKEAIGRLF | |
| HYQKRAGWAGK. | |
| ATGAAGCAATATTCAGTGGGTAATCAACATTCCAATTATAGGAGTCTCTTGTTTCCTTTCT | (SEQ ID NO:412) |
| GTGTTCACAGATGACACAGTTGACGGCCAGTGGGAATCAGACAATGGTGACTGAGTTGCT | |
| CTTCTCTATGTTCCCGCATGCGCACAGAGGTGGCCTCTTATTCTTTATTCCCTTGCTTCTCA | |
| TCTACGGATTTATCCTAACTGGAAACCTAATAATGTCATTGTCATCCAGGTGGGCATGGC | |
| CCTGCACACCCCTTTGTATTTCTTTATCAGTGTCCTCTCCTTCCTGGAGATCTGCTATACCA | |
| CAACCACCATCCCCAAGATGCTGTCCTGCCTAATCAGTGAGCAGAAGAGCATTTCCGTGGC | |
| TGGCTGCCTCCTGCAGATGTACTTTTTCCACTCACTTGGTATCACAGAAAGCTGTGTCCTG | |
| ACAGCAATGGCCATTGACAGGTACATAGCTATCTGCAATCCACTCCGTTACCCAACCATCA | |
| TGATTCCCAAACTTTGTATCCAGCTGACAGTTGGATCCTGCTTTTGTGGCTTCCTCCTTGTG | |
| CTTTCCTGAGATTGCATGGATTTCCACCTTGCCTTTCTGTGGCTCCAACCAGATCCACCAGAT | |
| ATTCTGTGATTTCACAGCTGTGCTGAGCTTGGCCTGCACAGATACATTCCTAGTGGTCATT | |
| GTGGATGCCATCCATGCAGCGGAAATTGTAGCCTCCTTCCTGGTCATTGCTCTATCCTACA | |
| TCCGGATTATTATAGTGATTGTGGGAATGCACTCAGCTGAAGGTCATCACAAGGCCTTTTTC | |
| CACCTGTGCTGCTCACCTTGCTGTGTTCTTGCTATTTTTTGGCAGTGTGGCTGTCATGTATT | |
| TGAGATTCTGAGCCACCTACTCAGTGTTTTGGGACACAGCAATTGCTGTCACTTTTGTTATC | |
| CTTGCTCCCTTTTTCAACCCCATCATCTATAGCCTGAAAAACAAGGACATGAAAGAGGCTA | |
| TTGGAAGGCTTTTCCACTATCAGAAGAGGGCTGGTTGGGCTGGGAAATAG. | |
| AOLFR221 sequences: | |
| MRNLSGGHVEEFVLVGFPTTPPLQLLLFVLFFAITLLTLLENALIVFTIWLAPSLHRPMTTFLGH | (SEQ ID NO:413) |
| LSFLELWTNVTIPRLLAAFLTQDGRVSTVGCMTQLTTFIALACTECVLLAVMATDRTLAICGP | |
| LLYPSLMPSSLATRLAAASWGSGFFSSMMKLLFISQLSTCGPNIINHFFCDISPLLNLTCSDKEQA | |
| ELVDFLLALVMILLPLLAVVSSTTAIIAAILRIPTSRGRHKAFSTCAAHLAVVVITTSSTLFTTAR | |
| PRAMYTFNHNKHSVLTTIIVPFFNPAITCLRNKEVKEAFRKTVMGRCHTPRDVQD. | |
| ATGAGAAATTTGAGTGGAGGCCATGTCGAGGAGTTTGTCTTGGTGGGTTTCCCTACCACGC | (SEQ ID NO:414) |
| CTCCCCTCCAGCTGCTCCTCTTTGTCCTTTTTTTTGCAATTTACCTTCTGACATTGTTGGAGA | |
| ATGCACTTATTGTCTTCACAATATGGCTTGCTCCAAGCCTTCATCGTCCCATGTACTTTTTC | |
| CTTGGCCATCTCTCTTTCCTGGAGCTATGGTACATCAATGTCACCATTCCTCGGCTCTTGGC | |
| AGCCTTTCTTACCCAGGATGGTAGAGTCTCCTACGTAGGTTGCATGACCCAACTGTACTTC | |
| TTTATTGCCTTAGCCTGTACTGAATGTGTGCTGTTGGCAGTTATGGCCTATGATCGCTACCT | |
| GGCCATCTGTGGACCCCTCCTTTACCCTAGTGTCATGCCTTCCAGTCTGGCCACTCGGCTTG | |
| CTGCTGCCTCTTGGGGCAGTGGCTTCTTCAGCTCCATGATGAAGCTTCTTTTTATTTCCCAA | |
| TTGTCCTACTGTGGACCCAACATTATCAACCACTTTTTCTGTGATATTTCCCCACTACTCAA | |
| CCTCACCTGCTCTGACAAGGAGCAAGCAGAGCTAGTAGACTTCCTTCTGGCCCTGGTGATG | |
| ATTCTACTCCCTCTATTGGCTGTGGTTTCATCATACACTGCCATCATTGCAGCCATCCTGAG | |
| GATCCCTACGTCCAGGGGACGCCACAAAGCCTTTTCCACTTGTGCCGCTCATCTGGCAGTG | |
| GTTGTTATCTACTACTCCTCCACTCTCTTCACCTATGCACGGCCCCGGGCCATGTACACCTT | |
| CAACCACAACAAGATTATCTCTGTGCTCTACACTATCATTGTACCATTCTTCAACCCAGCCA | |
| TCTACTGCCTGAGGAACAAGGAGGTGAAGGAGGCCTTCAGGAAGACAGTGATGGGCAGAT | |
| GTCACTATCCTAGGGATGTTCAGGACTGA. | |
| AOLFR222 sequences: | |
| MGQTNVTSWRDFVFLGFSSSGELQLLLFALFLSLTLVTLTSNVFIIIAIRLDSHLHTPMTLFLSFL | (SEQ ID NO:415) |
| SFSETCTTLGIIPRMLSGLAGGDQAISTVGCAAQMFFSASWACTNCFLLAAMGFDRTVAICAPL | |
| HYASHMNPTLCAQLVITSFLTGTLFGLGMTLVIIFHLSFCSSHEIQHFFCDTPPVLSLAGGDTGPS | |
| ELRIFILSLLVLLVSFFFITISTATLAAILRIPSAEGQKLKAFSTCASHLTVVIIHTGCASFVTLRPK | |
| ASYSLERQLIAMTTTVVTPLLNPIVTSLRTRAIQTALRNAFRGRLLGKG. | |
| ATGGGGCAGACCAACGTAACCTCCTGGAGGGATTTTGTCTTCCTGGGCTTCTCCAGTTCTG | (SEQ ID NO:416) |
| GGGAGTTGCAGCTCCTTCTCTTTGCCTTGTTCCTCTCTCTGTATCTAGTCACTCTGACCAGC | |
| AATGTCTTCATTATCATAGCCATCAGGCTGGATAGCCATCTGCACACCGCCATGTACCTCTT | |
| CCTTTCCTTCCTATCCTTCTCTGAGACCTGCTACACTTTGGGCATCATCCCTAGAATGCTCT | |
| CTGGCCTGGCTGGGGGGGACCAGGCTATGTCCTATGTGGGCTGTGCTGCCCAGATGTTCTT | |
| TTGTGCCTCATGGGCCTGTACTAACTGCTTCCTTCTGGCTGCCATGGGCITTGACAGATATG | |
| TGGCCATCTGTGCTCCACTCCACTATGCCAGCCACATGAATCCTACCCTCTGTGCCCAGCT | |
| GGTCATTACTTCCTTCCTGACTGGATACCTCTTTGGACTGGGAATGACACTAGTTATTTTCC | |
| ACCTCTCATTCTGCAGCTCCCATGAAATCCAGCACTTTTTTTGTGACACGCCACCTGTGCTG | |
| AGCCTAGCCTGTGGAGATACAGGCCCGAGTGAGGTGAGGATCTTIATCCTCAGTCTTTTGG | |
| TCCTCTTGGTCTCCTTCTTCTTCATCACCATGTCCTACGCCTAGATCTTGGCAGCAATACTG | |
| AGGATCCCCTCTGCTGAGGGGCAGAAGAAGGCCTTCTCCACTTGTGCCTCGGACCTTACAG | |
| TGGTCATTATTCATTATGGCTGTGCTTCCTTCGTGTACCTGAGGCCCAAAGCCAGCTACTCT | |
| CTTGAGAGAGATCAGCTTATTGCCATGACGTATACTGTAGTGACCCCCCTCCTTAATCCCA | |
| TTGTTTATAGTCTAAGGACTAGGGCTATACAGACAGCTCTGAGGAATGCTTTCAGAGGGAG | |
| ATTGCTGGGTAAAGGATGA. | |
| AOLFR223 sequences: | |
| MEAANESSEGISFVLLGLTTSPGQQRPLFVLFLLLTVASLLGNGLIVAAIQASPALHAPMYFLLA | (SEQ ID NO:417) |
| HLSFADLCFASVTVPKMLANLLAHDHSISLAGCLTQMTTFFALGVTDSCLLAAMATDCTVAIR | |
| HPLPTATRMSRAMCAALVGMAWLVSHVHSLLTLLMARLSFGASHQVPHFFCDHQPLLRLSC | |
| SDTHHIQLLIFTEGAAVVVTPFLLILASTGAIAAAVLQLPSASGRLRAVSTCGSHLAVVSLFYGT | |
| VIAVYFQATSRREAEWGRVATVMTTVVTPMLNPIITSLWNRDVQGALRALLIGRRISASDS. | |
| ATGGAGGCTGCCAATGAGTCTTCAGAGGGAATCTCATTCGTTTTATTGGGACTGACAACAA | (SEQ ID NO:418) |
| GTCCTGGACAGCAGCGGCCTCTCTTTGTGCTGTTCTTGCTCTTGTATGTGGCCAGCCTCCTG | |
| GGTAATGGACTCATTGTGGCTGCCATCCAGGCCAGTCCAGCCCTTCATGCACCCATGTACT | |
| TCCTGCTGGCCCACCTGTCCTTTGCTGACCTCTGTTTCGCCTCCGTCACTGTGCCCAAGATG | |
| TTGGCCAACTTGITGGCCCATGACCACTCGATCTCGCTGGCTGGCTGCCTGACCCAAATGT | |
| ACTTCTTCTTTGCCCTGGGGGTAACTGATAGCTGTCTTCTGGCGGCCATGGCCTATGACTG | |
| CTACGTGGCCATCCGGCACCCCCTCCCCTATGCCACGAGGATGTCCCGGGCCATGTGCGCA | |
| GCCCTGGTGGGAATGGCATGGCTGGTGTCCCACGTCCACTCCCTCCTGTATATCCTGCTCA | |
| TGGCTCGCTTGTCCTTCTGTGCTTCCCACCAAGTGCCCGACTTCTTCTGTGACCACCAGCCT | |
| CTCTTAAGGCTCTCGTGCTCTGACACCCACCACATCCAGCTGCTCATCTTCACCGAGGGCG | |
| CCGCAGTGGTGGTCACTCCCTTCCTGCTCATCCTCGCCTCCTATGGGGCCATCGCAGCTGC | |
| CGTGCTCCAGCTGCCCTCAGCCTCTGGGAGGCTCCGGGCTGTGTCCACCTGTGGCTCCCAC | |
| CTGGCTGTGGTGAGCCTCTTCTATGGGACAGTCATTGGAGTCTACTTCCAGGCCACATCCC | |
| GACGCGAGGCAGAGTGGGGCCGTGTGGCCACTGTCATGTACACTGTAGTCACCCCCATGC | |
| TGAACCCCATCATCTACAGCCTCTGGAATCGCGATGTACAGGGGGCACTCCGAGCCCTTCT | |
| CATTGGGCGAAGGATCTCAGCTAGTGAGTCCTGA. | |
| AOLFR224 sequences: | |
| MGSFNTSFEDGFILVGFSDWPQLEPILFVFIFIFTSLTLFGNTIIIALSWLDLRIHTPMTTFLSHLSL | (SEQ ID NO:419) |
| LDLCFTTSTVPQLLINLCGVDRTTRGGCVAQLFITLAIGSTECVLLVVIVIAFDRTAAVCRPLHY | |
| MAIMHPHLCQTLAIASWGAGFVNSLIQTGLAMAMPLCGHRLNHFFGEMPVFLKLACADTEGT | |
| EAKMFVARVIVVAVPAALILGSTVIHIAHAVLRVKSTAGRRTTAFGTCGSHLLVVFLFTGSAIYT | |
| YLQSIHNTSEREGKFVALFTTIITPILNPLITTLRNKDVKGALWKVLWRGRDSG. | |
| ATGGGAAGTTTCAACACCAGTTTTGAAGATGGCTTCATTTTGGTGGGATTCTCAGATTGGC | (SEQ ID NO:420) |
| CGCAACTGGAGCCCATCCTGTTTGTCTTTATTTTTATTTTCTACTCCCTAACTCTCTTTGGC | |
| AAGACCATCATCATCGCTCTCTCCTGGCTAGACCTTCGGCTGCACACACCTATGTACTTCTT | |
| TCTCTCTCATCTGTCCCTCCTGGACCTCTGCTTCACCACCAGCACCGTGCCCCAGCTCCTGA | |
| TCAACCTTTGCGGGGTGGACCGCACCATCACGCGTGGAGGGTGTGTGGCTCAGCTCTTCAT | |
| CTACCTAGCCCTGGGCTCCACAGAGTGTGTGCTCCTGGTGGTGATGGCCTTTGACCGCTAT | |
| GCTGCTGTCTGTCGTCCACTCCACTACATGGCCATCATGGACCCCCATCTCTGCCAGACCCT | |
| GGCTATCGCCTCCTGGGGTGCGGGTTTCGTGAACTCTCTGATCCAGACAGGTCTCGCAATG | |
| GCCATGCCTCTCTGTGGCCATCGACTGAATCACTTCTTCTGTGAGATGCCTGTATTTCTGAA | |
| GTTGGCTTGTGCGGACACAGAAGGAACAGAGGCCAAGATGTTTGTGGCCCGAGTCATAGT | |
| CGTGGCTGTTCCTGCAGCACTTATTCTAGGCTCCTATGTGCACATTGCTCATGCAGTGCTG | |
| AGGGTGAAGTCAACGGCTGGGCGCAGAAAGGCTTTGGGACTTGTGGGTCCCACCTCCTA | |
| GTAGTTTTCCTTTTTTATGGCTCAGCCATCTACACATATCTCCAATCCATCCACAATTATTC | |
| TGAGCGTGAGGGAAAATTTGTTGCCCTTTTTTATACTATAATTACCCCCATTCTCAATCCTC | |
| TCATTTATACACTAAGAAACAAGGACGTGAAGGGGGGTCTGTGGAAAGTACTATGGAGGG | |
| GGAGGGACTCAGGGTAG. | |
| AOLFR225 sequences: | |
| MENYNQTSTDFILLGLFPPSIIDLFFFILIVFIFLMALIGNLSMILLIFLDTHLHTPMTTLLSQLSLID | (SEQ ID NO:421) |
| LNYSTIVPKMASDFLHGNKSISFTGCGIQSFFFLALGGAEALLLASMATDRTAIGFPLHTLIRM | |
| SKRVCVLMITGSWIIGSINACAIH1VTVLHIPTCRSRAIMHFFCDVPAMVTLACMDTWVTEGTV | |
| FLSATTFLVFPFIGISCSTGQVLFAVTHMKSAEGRKKATLTCSTHLTVVTFTTAPFVTTTLRPRS | |
| LRSPTEDKVLAVFTTILTPMLNPIITSLRNKEVMGALTRVSQRICSVKM. | |
| ATGGAAAATTACAATCAAACATCAACTGATTTCATCTTATTGGGGCTGTTTCCACCATCAA | (SEQ ID NO:422) |
| TAATTGACCTTTTCTTCTTCATTCTCATTGTTTTCATTTTCCTGATGGCTCTAATTGGAAACC | |
| TGTCCATGKTPCTTCTCATCTTCTTGGACACCCATCTCCACACACCCATGTATTTCCTACTG | |
| AGTGAGCTCTCCCTCATTTGAGCTAAATTACATCTCCACCATTGTTCCTAAGATGGCATCTGA | |
| TTTTCTGCATGGAAACAAGTCTATCTCCTTCACTGGGTGTGGGATTCAGAGTTTCTTCTTCT | |
| TGGCATTAGGAGGTGCAGAAGCACTACTTTTGGCATCTATGGCCTATGATCGTTACATTGC | |
| TATTTGCTTTCCTCTCCACTATCTCATCCGCATGAGCAAAAGAGTGTGTGTGCTGATGATA | |
| ACAGGGTCTTGGATCATAGGCTCGATCAATGCTTGTGCTCACACTGTATATGTACTCCATA | |
| TTCCTTATTGCCGATCCAGGGCCATCAATCATTTCTTCTGTGATGTCCCAGCAATGGTGACT | |
| CTGGCCTGCATGGACACCTGGGTCTATGAGGGCACAGTGTTTTTGAGTGCCACCATCTTTTC | |
| TCGTGTTTCCCTTCATTGGTATTTCATGTTCCTATGGCCAGGTTCTCTTTGCTGTCTACCAC | |
| ATGAAATCTGCAGAAGGGAGGAAGAAAGCCTATTTGACCTGCAGCACCCACCTCACTGTA | |
| GTAACTTTCTACTATGCACCTTTTGTCTACACTTATCTACGTCCAAGATCCCTGCGATCTCC | |
| AACAGAGGACAAGGTTCTGGCTGTCTTCTACACCATCCTCACCCCAATGCTCAACCCCATC | |
| ATCTATAGCCTGAGGAACAAGGAGGTGATGGGGGCCGTGACACGAGTGAGTCAGAGAATC | |
| TGCTCTGTGAAAATGTAG. | |
| AOLFR226 sequences: | |
| MEWRNHSGRVSEFVLLGFPAPAPLQVLLFALLLLATVLVLTENTLIIMAIPTHSTLIHKTMYFFL | |
| ANMSFLEIWTVTVTIPKMLAGFVGSKQDHGQLISFEGCMTQLTTFLGLGCTECVLLAVMAYD | |
| RYMAICTPLHTPVIVSGRLCVQMAAGSWAGGFGISMVKVFLISGLSTCGPNIINHFFCDVSPLL | |
| NLSCTDMSTAELTDFILAIFILLGPLSVTGASTVATTGAVMHISSAAGRTKAFSTCASHLTVVIIF | |
| YAASIFITARPKALSAEDThIKLVSVLTAVIVPLLNPIITCLRNQEVKRALCCTLHLTQHQDPDP | |
| KKASRNV (SEQ ID NO:423). | |
| ATGGAGTGGCGGAACCATAGTGGGAGAGTGAGTGAGTTTGTGTTGCTGGGCTTCCCTGCT | (SEQ ID NO:424) |
| CCTGCGCCACTACAGGTACTATTGTTTGCCCTTTTGCTGCTGGCCTATGTGTTGGTGCTGAC | |
| TGAGAACACACTCATCATTATGGCAATTAGGAACCATTCTACCCTCCACAAACCCATGTAC | |
| TTTTTTCTAGCTAATATGTCCTTTCTGGAGATCTGGTATGTCACTGTCACTATTCCCAAGAT | |
| GCTTGCTGGCTTTGTTGGATCCAAACAGGATCATGGACAGCTAATCTCCTTTGAGGGATGC | |
| ATGACACAGCTCTACTTTTTCCTTGGCTTGGGCTGCACTGAGTGTGTCCTTCTCGCTGTTAT | |
| GGCCTATGATCGCTATATGGCCATCTGCTATCCTCTCCACTACCCAGTCATTGTCAGTGGCC | |
| GGCTGTGTGTGCAGATGGCTGCTGGCTCTTGGGCTGGAGGTTTTGGCATCTCCATGGTCAA | |
| AGTTTTTCTTATTTCTGGCCTCTCTTACTGTGGCGCCAACATCATCAACCACTTTTTCTGTG | |
| ATGTCTCTCCATTGCTCAACCTCTCATGCACTGATATGTCCACAGCAGAGCTTTACAGATTTC | |
| ATCCTGGCCATTTTTATTCTTCTAGGGCCACTCTCTGTCACTGGGGCCTCCTATGTGGCCAT | |
| TACTGGTGCTGTGATGCACATATCTTCGGCTGCTGGACGCTATAAGGCCTTTTCCACCTGT | |
| GCCTCTCATCTCACTGTTGTGATAATCTTCTATGCAGCCAGTATCTTCATCTATGCTCGGCC | |
| AAAGGCACTCTCAGCTTTTGACACCAACAAGTTGGTCTCTGTACTGTATGCTGTCATTGTA | |
| CCATTGCTCAATCCCATCATTTACTGCCTGCGCAATCAAGAGGTCAAGAGAGCCCTATGCT | |
| GTACTCTGCACCTGTACCAGCACCAGGATCCTGACCCCAAGAAAGCTAGCAGAAATGTATA | |
| G. | |
| AOLFR227 sequences: | |
| MEPQNTSTVTNFQLLGFQNLLEWQALLFVIFLLITCLTIIGNVVIITVVSQGLRLHSPMTMFLQH | (SEQ ID NO:425) |
| LSFLEVWTTSTTVPLLLANLLSWGQMSFSACMAQLTTFVFLGATECFLLAFMATDRTLAICSP | |
| LRYPFLMHRGLCARLVVVSWCTGVSTGFLHSMMISRLDFCGRNQINIIFFCDLPPLMQLSCSRV | |
| YITEVTIFILSIAVLCICFFLTLGPTVFIVSSILRIPSTSGRRKTFSTCGSHLAVVTLTTGTMISMYV | |
| CPSPHLLPEINTISVFTTVVTPLLNPVITSLRINKDFKEAVRKVMRRKCGILWSTSKRKF | |
| LY. | |
| ATGGAGCCCCAAAATACCTCCACTGTGACTAACTTTCAGCTGTTAGGATTCCAGAACCTTC | (SEQ ID NO:426) |
| TTGAATGGCAGGCCCTGCTCTTTGTCATTTTCCTGCTCATCTACTGCCTGACCATTATAGGG | |
| AATGTTGTCATCATCACCGTGGTGAGCCAGGGCCTGCGACTGCACTCCCCTATGTACATGT | |
| TCCTGCAGGATCTCTCCTTTCTGGAGGTCTGGTACACGTCCACCACTGTGCCCCTTCTCCTA | |
| GCCAACCTGCTGTCCTGGGGCCAAGCCATCTCGTTCTCTGCCTGCATGGCACAGCTCTACT | |
| TCTTCGTATTCCTCGGCGCCACCGAGTGCTTTCTCCTGGCCTTCATGGCCTATGACCGTTAC | |
| CTGGCCATCTGCAGCCCACTCCGCTACCCCTTTTCTCATGCATCGTGGGCTATGTGCCAGGTT | |
| GGTGGTGGTCTCATGGTGCACAGGGGTCAGCACAGGCTTTCTGCATTCGATGATGATTTCC | |
| AGGTTGGACTTCTGTGGGCGCAATCAGATTAACCATTTCTTCTGCGACCTCCCGCCACTCA | |
| TGCAGCTCTCCTGTTCCAGAGTTTATATCACCGAGGTGACCATCTTCATCCTGTCAATTGCC | |
| GTGCTGTGCATTTGTTTTTTTCTGACACTGGGGCCCTATGTTTTCATTGTGTCCTCCATATT | |
| GAGAATCCCTTCCACCTCTGGCCGGAGAAAGACCTTTTCCACATGTGGCTCCCACCTGGCT | |
| GTTGTCACTCTCTACTACGGGACCATGATCTCCATGTATGTGTGTCCCAGTCCCCACCTGTT | |
| GCCTGAAATCAACAAGATCATTTCTGTCTTCTACACTGTGGTCACACCACTGCTGAACCCA | |
| GTTATCTACAGCTTGAGGAACAAAGACTTCAAAGAAGCTGTTAGAAAGGTCATGAGAAGG | |
| AAATGTGGTATTCTATGGAGTACAAGTAAAAGGAAGTTCCITTATTAG. | |
| AOLFR229 sequences: | |
| MFYVNQIPFQLTHISFVTPTELWSRAHPCMPTLSFWVCSATPVSPGFFALILLVFVTSIASNVVK | (SEQ ID NO:427) |
| IILIHIDSRLHTPMTTLLSQLSLRDILTSTIVPKMLVDQVMSQRAISFAGCTAQHFLTLTLAGAE | |
| FFLLGLMSCDRTVAICNPLHTPDLMSRTCWLIVAAAWLGGSIDGFLLTPVTMQFPFCASREIN | |
| HFFCEVPALLKLSCTDTSATETAMTVCCIMMLLIPFSVISGSTTRILITKTTRTTASEAEGRRKAVAT | |
| CSSHMVVVSLFTGAAMTTTVLPHSTHTPEQDKAVSAFTTILTPMLNPLITSLRNKDVTGALQK | |
| VVGRCVSSGKKITF. | |
| ATGTTTTATGTAAATCAGATACCTTTCCAACTTTATCATATCTCTTTCGTGTACCCTACAGA | (SEQ ID NO:428) |
| GCTATGGAGCAGAGCAATTATTCCGTGTATGCCGAGTTTATCCTTCTGGGTTTGTTCAGCA | |
| ACGCCCGTTTCCCCTGGCTTCTTTGGCCTCATTCTCCTGGTCTTTGTGACCTCCATAGCCAG | |
| CAACGTGGTCAAGATCATTCTCATCCACATAGACTCCCGCCTCCACACCCCCATGTACTTC | |
| CTGCTCAGCCAGCTCTCCCTCAGGGACATCCTGTATATTTCCACCATTGTGCCCAAAATGCT | |
| GGTCGACCAGGTGATGAGCCAGAGAGCCATTTCCITTGCTGGATGCACTGCCCAACACTTC | |
| CTCTACTTGACCTTAGCAGGGGCTGAGTTTCTTCCTCCTAGGACTCATGTCCTGTGATCGCTA | |
| CGTAGCCATGTGCAACCCTCTGCACTATCCTGACCTCATGAGCCGCAAGATCTGCTGGTTG | |
| ATTGTGGCGGCAGCCTGGCTGGGAGGGTCTATCGATGGTTTCTTGCTCAGCCCCGTCACCA | |
| TGCAGTTCCCCTTCTGTGCCTCTCGGGAGATCAAGCACTTCTTCTGCGAGGTGCCTGCCCTT | |
| CTGAAGCTCTCCTGCACGGACACATCAGCCTACGAGACAGCCATGTATGTCTGCTGTATTA | |
| TGATGCTCCTCATCCCTTTCTCTGTGATCTCGGGCTCTTACACAAGAATTCTCATTACTGTT | |
| TATAGGATGAGCGAGGCAGAGGGGAGGCGAAAGGCTGTGGCCACCTGCTCCTCACACATG | |
| GTGGTTGTCAGCCTCTTCTATGGGGCTGCCATGTACACATACGTGCTGCCTCATTCTTACCA | |
| CACCCCTGAGCAGGACAAAGCTGTATCTGCCTTCTACACCATCCTCACTCCCATGCTCAAT | |
| CCACTCATTTACAGCCTTAGGAACAAGGATGTCACGGGGGCCCTACAGAAGGTTGTTGGG | |
| AGGTGTGTGTCCTCAGGAAAGGTAACCACTTTCTAA. | |
| AOLFR23 sequences: | |
| MGMEGLLQNSTNFVLTGLITHPAFPGLLFAIVFSIFVVATTANLVMILLIHMDSRLHTPMYFLLS | (SEQ ID NO:429) |
| QLSIMDTITCITVPKMLQDLLSKDKTISFLGCAVQIFLTLTLIGGEFFLLGLMATDRTVAVCNP | |
| LRYPLLMNRRVCLFMVVGSWVGGSLDGFMLTPVTMSFPFCRSREINHFFCEIPAVLKLSCTDTS | |
| LYETLMTACCVLMLLIPLSVISVSTTHILLTVHRMNSAEGRRKAEATCSSHIMVVSVFTGAAFY | |
| TNVLPHSTHTPEKDKVVSAFTTILTPMLNPLITSLRNKDVAAALRKVLGRCGSSQSLRVATVIR | |
| KG. | |
| ATGGGCATGGAGGGTCTTCTCCAGAACTCCACTAACTTCGTCCTCACAGGCCTCATCACGC | (SEQ ID NO:430) |
| ATCCTGCCTTCCCCGGGCTTCTCTTTGCAATAGTGTTCTCCATCTTTGTGGTGGCTATAACA | |
| GCCAACTTGGTCATGATTCTGCTCATCCACATGGACTCCCGCCTCCACACACCCATGTACTT | |
| CTTGCTCAGCCAGCTCTCCATCATGGATACCATCTACATCTGTATCACTGTCCCCAAGATGC | |
| TCCAGGACCTCCTGTCCAAGGACAAGACCATTTCCTTCCTGGGCTGTGCAGTTCAGATCTT | |
| CCTCTACCTGACCCTGATTGGAGGGGAATTCTTCCTGGTGGGTCTCATGGGCTATGACCGC | |
| TATGTGGCTGTGTGCAACCCTCTACGGTACCCTCTCCTCATGAACCGCAGGGTTTGCTTATT | |
| CATGGTGGTCGGCTCCTGGGTTGGTGGTIUCCTTGGATGGGTTCATGCTGACTCCTGTCACT | |
| ATGAGTTUTCCCCTTCTGTAGATCCCGAGAGATCAATCACTTTTTCTGTGAGATCCCAGCCGT | |
| GCTGAAGTTGTCTTGCAGAGACACGTCACTCTATGAGACCCTGATGTATGCCTGCTGCGTG | |
| CTGATGCTGCTTATCCCTCTATCTGTCATCTCTGTCTCCTACACGCACATCCTCCTGACTGT | |
| CCACAGGATGAACTCTGCTGAGGGCCGGCGCAAAGCCTTTGCTACGTGTTCCTCCCAGATT | |
| ATGGTGGTGAGCGTTTTCTACGGGGCAGCCTTCTACACCAACGTGCTGCCCCACTCCTACC | |
| ACACTCCAGAGAAAGATAAAGTGGTGTCTGCCTTGTACACCATCCTCACCCCCATGCTCAA | |
| CCCACTCATCTACAGCTTGAGGAATAAAGATGTGGCTGCAGCTCTGAGGAAAGTACTAGG | |
| GAGATGTGGTTCCTCCCAGAGCATCAGGGTGGCGACTGTGATCAGGAAGGGCTAG. | |
| AOLFR231 sequences: | |
| MERANHSVVSEFILLGLSKSQNLQILFFLGFSVVFVGIVLGNLLILVTVTFDSLLHTPMTTLLSNL | (SEQ ID NO:431) |
| SCIDMILASFATPKMIVDFLRERKTISWWGCTSQMFFMHLLGGSEMMLLVAMAIDRTVAICKP | |
| LHYMTIMSPRVLTGLLLSSTAVGFVHSSSQMAiFMLTLPFCGPNVIDSFFCDLPLVIKLACKDTYI | |
| LQLLVIADSGLLSLVCFLLLLVSTGVIIFSVRTRAASRSSKAFSTLSAHITVVTLFFAPCVFIYVW | |
| PFSRTSVDTLSVFTTIFTPLLNPIITTLRNQEVKAAIKKRLCI. | |
| ATGGAAAGAGCAAACCATTCAGTGGTATCGGAATTTATTTTGTTGGGACTTTCCKAATCTC | (SEQ ID NO:432) |
| AAAATCTTGAGATTTTATTCTTCTTGGGATTCTCTGTGGTCTTCGTGGGGATTGTGTTAGGA | |
| AACCTGCTCATCTTGGTGAGTGTGACCTTTGATTCGCTCCTTTCACACACCAATGTATTTTCT | |
| GCTTAGCAACCTCTCCTGCATTGATATGATCCTGGCTTCTTTTGCTACCCCTAAGATGATTG | |
| TAGATTTCCTCCGAGAACGTAAGACCATCTCATGGTGGGGATGTTATTCCCAGATGTTCTT | |
| TATGCACCTCCTGGGTGGGAGTGAGATGATGTTGCTTGTAGCCATGGCAATAGACAGGTAT | |
| GTTGCCATATGCAAACCCCTCCATTACATGACCATCATGAGCCCACGGGTGCTCACTGGGC | |
| TACTGTTATCGTCCTATGCAGTTGGATTTGTGCACTCATCTAGTCAAATGGCTTTCATGTTG | |
| ACTTTGCCCTTCTGTGGTCCCAATGTTATAGACAGCTTTTTCTGTGACCTTCCCCTTGTGAT | |
| TAAACTTGCCTGCAAGGACACCTACATCCTACAGCTCCTGGTCATTGCTGACAGTGGGCTC | |
| CTGTCACTGGTCTGCTTCCTCCTCTTGCTTGTCTGCTATGGAGTCATAATATTCTGAGTTAG | |
| GTACCGTGCTGCTAGTCGATCCTCTAAGGCTTTCTCCACTCTCTCAGCTCACATCACAGTTG | |
| TGACTGTGTTCTTTGCTCCGTGTGTCTTTATCTACGTCTGGCCCTTCAGCAGATACTCGGTA | |
| GATAAAATTCTTTCTGTGTTTTTACACAATTTTCACACCTCTCTTAAATCCTATTATTTATAC | |
| ATTAAGAAATCAAGAGGTAAAAGCAGCCATTAAAAAAAGACTCTGCATATAA. | |
| AOLIFR232 sequences: | |
| MDNITWMASHTGWSDFILMGLFRQSKHPMANITWMANHTGWSDFILLGLFRQSTIPAILCV | (SEQ ID NO:433) |
| VIFVVFLMALSGNAVLILLIHCDAIILHTPMTTFISQLSLMDMATSVTVPKMLLDQVMGVNKIS | |
| APECGMQMFFTVTLAGSEFFLLATMATDRTVAICHPLRTPVLMNHRVCLFLSSGCWFLGSVD | |
| GFTFTPITMTFPFRGSREIHHFFCEVPAVLNLSCSDTSLTEIFMTLCCVLMLLIPVVIISSSTLLILL | |
| TIHGMNSAEGRKKAFATCSSHLTVVILFTGAAITTTMLPSSTHTPEKDMMVSVFTTILTPVVNP | |
| LIYSLRNKDVMGALKKMLTVEPAFQKAME. | |
| ATGGACAACATCACCTGGATGGCCAGCCACACTGGATGGTCGGATTTCATCCTGATGGGAC | (SEQ lID NO:434) |
| TCTTCAGACAATCCAAACATCCAATGGCCAATATCACCTGGATGGCCAACCACACTGGATG | |
| GTCGGATTTCATCCTGTTGGGACTCTTCAGACAATCCAAACATCCAGCACTACTTTGTGTG | |
| GTCATPTTTGTGGTTTTCCTGATGGCGTTGTCTGGAAATGCTGTCCTGATCCTTCTGATACA | |
| CTGTGACGGGCACCTCCACACCCCCATGTACTTTTTCATCAGTCAATTGTCTCTCATGGACA | |
| TGGCGTACATTTCTGTCACTGTGCCCAAGATGCTCCTGGACCAGGTCATGGGTGTGAATAA | |
| GATCTCAGCGCCTGAGTGTGGGATGCAGATGTTCTTCTACGTGACACTAGCAGGTTCAGAA | |
| TTTTTCCTTCTAGCCACCATGGCCTATGACCGCTACGTGGCCATCTGCCATCCTCTCCGTTA | |
| CCCTGTCCTCATGAACCATAGGGTGTGTCTCTTCCTGTCATCAGGCTGCTGGTTGCTGGGCT | |
| CAGTGGATGGCTTCACATTCACTCCCATCACCATGACCTTCCCCTTCCGTGGATCCCGGGA | |
| GATTCATCATTTCTTCTGTGAAGTTCCTGCTGTATTGAATCTCTGCTGCTCAGACACCTCAC | |
| TCTATGAGATTTTCATGTACTTGTGCTGTGTCCTCATGCTCCTCATCCCTGTGGTGATCATT | |
| TCAAGCTCCTATTTACTCATCCTCCTCACCATCCACGGGATGAACTCAGCAGAGGGCCGGA | |
| AAAAGGCCTTTGCCACCTGGTCCTCCCACCTGACTGTGGTCATCCTCTTCTATGGGGCTGCC | |
| ATCTACACCTACATGCTGCCCAGCTCCTACCACACCCCTGAGAAGGACATGATGGTATCTG | |
| TCTTCTATACCATCCTCAGTCCAGTGGTGAACCCTTTAATCTATAGTCTTAGGAATAAGGAT | |
| GTCATGGGGGCTCTGAAGAAAATGTTAACAGTGGAACCTGCCTTTCAAAAAGCTATGGAG | |
| TAG. | |
| AOLFR233 sequences: | |
| MANITRMANHTGKLDFILMGLFRRSKHPALLSVVIFVVFLKALSGNAVLILLIHCDAIILHSPMT | (SEQ ID NO:435) |
| FFISQLSLMDMATSVTVPKMLLDQVMGVNKVSAPECGMQMFLTLTLAGSEFFLLATMAYDR | |
| YVAICHPLRTPVLMNHRVCLFLASGCWFLGSVDGFMLTPITMSFPFCRSWEIHIIFFCEVPAVTI | |
| LSCSDTSLTETLMTLCGVLMLLIPVTIISSSTLLILLTVHRMNSAEGRKKAFATCSSHLTVVILFY | |
| GAAVYTYMLPSSTHTPEKDMMVSVFTTILTPVLNPLITSLRNKDVMGALTKMLTVRFVL. | |
| ATGGCCAACATCACCAGGATGGCCAACCACACTGGAAAGTTGGATTTCATCCTCATGGGAC | (SEQ ID NO:436) |
| TCTTCAGACGATCCAAACATCCAGCTCTACTTAGTGTGGTCATCTTTGTGGTTTTCCTGAAG | |
| GCGTTGTCTGGAAATGCTGTCCTGATCCTTCTGATACACTGTGACGCCCACCTCCACAGCC | |
| CCATGTACTTTTTTCATCAGTCAATTGTCTCTCATGGACATGGCGTACATTTCTGTCACTGTG | |
| CCCAAGATGCTCCTGGACCAGGTCATGGGTGTGAATAAGGTCTCAGCCCCTGAGTGTGGG | |
| ATGCAGATGTTCCTCTATCTGACACTAGCAGGTTCGGAATTTTTCCTTCTAGCCACCATGGC | |
| CTATGACCGCTACGTGGCCATCTGCCATCCTCTCCGTTACCCTGTCCTCATGAACCATAGG | |
| GTCTGTCTTTTCCTGGCATCGGGCTGCTGGTTCCTGGGCTCAGTGGATGGCTTCATGCTCAC | |
| TCCCATCACCATGAGCTTCCCCTTCTGCAGATCCTGGGAGATTCATCATTTCTTCTGTGAAG | |
| TCCCTGCTGTAAGGATCCTGTCCTGCTCAGACACCTCACTCTATGAGACCCTCATGTACCTA | |
| TGCTGTGTCCTCATGGTCCTCATCCCTGTGACGATCATTTCAAGCTCCTATTTACTCATCCT | |
| CCTCACCGTCCACAGGATGAACTCAGCAGAGGGCCGGAAAAAGGCCTTTGCCACCTGCTC | |
| CTCCCACCTGACTGTGGTCATCCTCTTCTATGGGGCTGCCGTCTACACCTACATGCTCCCCA | |
| GCTCCTACCACAGCCCTGAGAAGGACATGATGGTATCTGTCTTCTATACCATCCTCACTCC | |
| GGTGGTGAACCCTTTAATCTATAGTCTTAGGAATAAGGATGTCATGGGGGCTCTGAAGAAA | |
| ATGTTAACTGTGAGATTCGTGCTTTAG. | |
| AOLFR234 sequences: | |
| MPNSTTVMEFLLMRIFSDVWTLQILHSASFFMLTLVTLMGNILIVTVTTCDSSLIIMPMTTFLRN | (SEQ ID NO:437) |
| LSILDACTSVTVPTSCVNSLLDSTTISKAGCVAQVFLVVFFVTVELLFLTIMAHDRTVAVCQPL | |
| HYPVIVNSRIICIQMTLASLLSGLVTAGMHTGSTFQLPFCRSNVIHQFFCDIPSLLKLSCSDTFSNE | |
| VMIVVSALGVGGGCFIFIIRSTHIFSTVLGFPRGADRTKAFSTCIPHILVVSVFLSSCSSVTLRPP | |
| AIPAATQDLILSGFTSIMPPLFNPIITSLRINKQIKVAIKTMKRIFTSENV. | |
| ATGCCCAATTCAACCACCGTGATGGAATTTCTCCTCATGAGGTTTTCTGATGTGTGGACAC | (SEQ ID NO:438) |
| TACAGATTTTACATTCTGCATCCTTCTTTATGTTGTATTTGGTAACTCTAATGGGAAACATC | |
| CTCATTGTGACCGTCACCACCTGTGACAGCAGCCTTCACATGCCCATGTACTTCTTCCTCAG | |
| GAATCTGTCTATCTTGGATGCCTGCTACATTTCTGTTACAGTCCCTACCTCATGTGTCAATT | |
| CCCTACTGGACAGCACCACCATTTCTAAGGCGGGATGTGTAGCTCAGGTCTTTCCTCGTGGT | |
| TTTTTTTGTATATGTGGAGCTTGTGTTTCTCACCATTATGGCTCATGACCGCTATGTGGCTG | |
| TCTGCCAGCCACTTCACTACCCTGTGATCGTGAACTCTCGAATCTGCATCCAGATGAGACT | |
| GGCGTCGCTACTCAGTGGTCTTGTCTATGCAGGCATGGACACTGGCAGCACATTCCAGCTG | |
| CCCTTGTGTGGGTCGAACGTTATTCATCAATTCTTCTGTGACATCCCCTCTCTGCTGAAGCT | |
| CTCTTGCTCTGACACCTTCAGCAATGAGGTCATGATTGTTGTCTCTGCTCTGGGGGTAGGT | |
| GGGGGCTGTTTCATGTTTATCATCAGGTCTTACATTCACATCTTTTCGACCGTGCTCGGGTT | |
| TCCAAGAGGAGCAGACAGAACAAAGGCCTTTTCCACCTGCATCCCTCACATCCTGGTGGTG | |
| TCAGTCTTCCTCAGTTCATGCTCTTCTGTGTACCTCAGGCCACCTGCGATACCTGCAGCCAC | |
| CCAGGATCTGATCCTTTCTGGTTTTTATTCCATAATGCCTCCCCTCTTTAACCCTATTATTTA | |
| CAGTCTTAGAAATAAGCAAATAAAGGTGGCCATCAAGAAAATCATGAAGAGAATTTTTTA | |
| TTCAGAAAATGTGTAA. | |
| AOLFR235 sequences: | |
| MDGVNDSSLQGFVLMGISDHPQLEMIFFIAILFSTLLTLLGNSTIILLSRLEARLHTPMTTFLSNL | (SEQ ID NO:439) |
| SSLDLAFATSSVPQMLINLWGPGKTISTGGCITQLTVFLWLGATECILLVVMAFDRTVAVCRPL | |
| RYTAIMNPQLCWLLAVIACLGGLGNSVIQSTFTLQLPLCGHRRVEGFLCEVPAMIKLACGDTSL | |
| NQAVLNGVCTFFTAVPLSIIVISTCLIAQAVLTRSAEGRRKAFNTCLSHLLVVFLFTGSASYGY | |
| LLPAKNSKQDQGKFISLFTSLVTPMVNPLITTLRNMEVKGALRRLLGKGREVG. | |
| ATGGACGGGGTGAATGATAGCTCCTTGCAGGGGTTTGTTCTGATGGGCATATCAGACGATC | (SEQ ID NO:440) |
| CCCAGCTGGAGATGATCTTTTTTATAGCCATCCTCTTCTCCTATTTGCTGACCCTACTTGGG | |
| AACTCAACCATCATGTTGCTTTCCCGCCTGGAGGCCCGGCTCCATACACCCATGTACTTCTT | |
| CCTCAGCAACCTCTCCTCCTTGGACGTTGCTTTCGCTACTAGTTCAGTCCCCCAAATGCTGA | |
| TCAATTTATGGGGACCAGGCAAGACCATCAGCTATGGTGGCTGCATAACCCAGCTCTATGT | |
| CCCTTTGGCTGGGGGCCACCGAGTGCATCCTGCTGGTGGTGATGGCATTTGACCGCTAC | |
| GTGGCAGTGTGCCGGCCCCTCCGCTACACCGCCATCATGAACGCCCAGGTGTGCTGGCTGC | |
| TGGCTGTGATTGCCTGCCTGGGTGGCTTGGGCAACTCTGTGATCCAGTCAACATTCACTCT | |
| GCAGCTCCCATTGTGTGGGCACCGGAGGGTGGAGGGATTCCTCTGCGAGGTGCCTGCCAT | |
| GATCAAACTGGCCTGTGGCGACACAAGTCTCAACCAGGCTGTGCTCAATGGTGTCTGCACC | |
| TTCTTCACTGCAGTCCCACTAAGCATCATGGTGATCTCCTACTGCCTCATTGCTCAGGCAGT | |
| GCTGAAAATCCGCTCTGCAGAGGGGAGGCGAAAGGCGTTCAATACGTGCCTCTCCCATCT | |
| GCTGGTGGTGTTCCTCTTCTATGGCTCAGCCAGCTATGGGTATCTGCTTCCGGCCAAGAAC | |
| AGCAAACAGGACCAGGGCAAGTTCATTTCCCTGTTCTACTCGTTGGTCACACCCATGGTGA | |
| ATCCCCTCATGTACACGCTGCGGAACATGGAAGTGAAGGGCGCACTGAGGAGGTTGCTGG | |
| GGAAAGGAAGAGAAGTTGGCTGA. | |
| AOLFR236 sequences: | |
| MTSQERDTAITSINVSFVAKGMTSRSVCEKMTMTTENPNQTVVSHFFLEGLRTTAKHSSLFFL | (SEQ ID NO:441). |
| LFLLIYSITVAGNLLILLTVGSDSHLSLPMTHFLGHLSFLDACLSTVTVPKVMAGLLTLDGKVIS | |
| FEGCAVQLTCFHFLASTECFLTTVMATDRTLAICQPLHTPVAIWNRRMCAEMAGITWAIGATH | |
| AAIHTSLTFRLLTCGPCHTATTFCDIPPVLKLACTDTTINELVMLASIGIVAAGCLILIVISTTFIVA | |
| AVLRIRTAQGRQRAFSPCTAQLTGVLLTTVPPVCITLQPRSSEAGAGAPAVFTTIVTPMLNPFIY | |
| TLRNKEVKHALQRLLCSSFRESTAGSPPP. | |
| ATGACATCTCAGGAAAGGGATACAGCTATTTATTCCATTAATGTCAGTTTTGTTGCAAAGG | (SEQ ID NO:442). |
| GGATGACTAGCCGCTCTGTGTGTGAGAAGATGACCATGACAACGGAGAACCCCAACCAGA | |
| CTGTGGTGAGCCACTTCTTCCTGGAGGGTTTGAGGTACACCGCTAAACATTCTAGCCTCTT | |
| CTTCCTCCTCTTCCTCCTCATCTACAGCATCACTGTGGCTGGGAATCTCCTCATCCTCCTAA | |
| CTGTGGGCTCTGACTCTCACCTCAGCTTACCCATGTAGCACTTCCTGGGGCACCTCTCCTTC | |
| CTGGATGCCTGTTTGTCTACAGTGACAGTGCCCAAGGTCATGGCAGGCCTGCTGACTCTGG | |
| ATGGGAAGGTGATCTCCTTTGAGGGCTQTGCCGTACAGCTTTATTGCTTCCACTTTCTGGC | |
| CAGCACTGAGTGCTTCCTGTACACAGTCATGGCCTATGACCGCTATCTGGCTATCTGTCAA | |
| CCCCTGCACTACCCAGTGGCCATGAACAGAAGGATGTGTGCAGAAATGGCTGGAATCACC | |
| TGGGCCATAGGTGCCACGCACGCTGCAATCCACACCTCCCTCACCTTCCGCCTGCTCTACT | |
| GTGGGCCTTGCCACATTGCCTACTTCTTCTGGGACATACCCCCTGTCCTAAAGCTCGCCTGT | |
| ACAGACACCACCATTAATGAGCTAGTCATGCTTGCCAGCATTGGCATCGTGGCTGCAGGCT | |
| GCCTCATCCTCATCGTTATTTCCTACATCTPTCATCGTGGCAGCTGTGTTGCGCATCCGCACA | |
| GCCCAGGGCCGGCAGCGGGCCTTCTCCCCCTGCACTGCCCAGCTCACTGGGGTGCTCCTGT | |
| ACTACGTGCCACCTGTCTGTATCTACCTGCAGCCTCGCTCCAGTGAGGCAGGAGCTGGGGC | |
| CCCTGCTGTCTTCTACACAATCGTAACTCGAATGCTCAACCGATTCATTTACACTTTGCGGA | |
| ACAAGGAGGTGAAGCATGCTCTGCAAAGGCTTTTGTGCAGCAGCTTCCGAGAGTCTACAG | |
| CAGGCAGCGCACCCCCATAG. | |
| AOLFR237 sequences: | |
| MDQRNTTRVKEFTFLGITQSRELSQVLFTFLFLVTMTTLMGNFLIMVTVTCESHLHTPMYFLL | (SEQ ID NO:443) |
| RNLSILDICFSSITAPKVLIDLLSETKTISFSGCVTQMFFFHLLGGADVFSLSVMAFDRTAISKPL | |
| HYMTIMSRGRCTGLIVGFLGGGLVHSIAQISLLLPLPVCGPNVLDTFTCDVPQVLKLACTDTFT | |
| LELLMISTTh4GLVSWFVFFFLLISTTVILMMLRSHTGEGRRKAISTCTSHITVVTLHFVPCIYVYA | |
| RPFTALPTDTAISVTFTVISPLLNIPIITTLRNQEMKLAMRKLKRRLGQSERILIQ. | |
| ATGGATCAGAGAAATTACACCAGAGTGAAAGAATTTACCTTCCTGGGAATTACTCAGTCCC | (SEQ ID NO:444) |
| GAGAAGTGAGCGAGGTCTTATTTACCTTCCTGTTTTTGGTGTACATGACAACTCTAATGGG | |
| AAACTTCCTGATCATGGTTACAGTTACCTGTGAATCTCACCTTCATACGCCCATGTACTTCC | |
| TGCTCCGCAACCTGTCTATTCTTGACATCTGCTTTTCCTCGATCACAGCTCCTAAGGTCCTG | |
| ATAGATCTTCTATCAGAGACAAAAACCATCTCCTTCAGTGGCTGTGTCACTCAAATGTTCT | |
| TCTTCCACCTTCTGGGGGGAGGAGACGTTTTTTCTCTCTCTGTGATGGCGTTTGACCGCTAT | |
| ATAGCCATCTCCAAGCCCCTGCACTATATGACCATCATGAGTAGGGGGCGATGCACAGGCC | |
| TCATCGTGGGGTTCCTGGGTGGGGGGCTTGTCCACTCCATAGCGCAGATTTCTCTATTGCT | |
| CCCACTCCCTGTCTGTGGACCCAATGTTCTTGACACTTTCTACTGCGATGTCCCCCAGGTCC | |
| TCAAACTTGCCTGCACTGACACCTTCACTCTGGAGCTCCTGATGATTTGAAATAATGGGTT | |
| AGTCAGTTGGTTTGTATTGTTCTTTTCTCCTCATATCTTACACGGTCATCTTGATGATGCTGA | |
| GGTCTCACACTGGGGAAGGCAGGAGGAAAGCCATCTCCACCTGCACCTCCCACATCACCG | |
| TGGTGACCCTGCATTTCGTGCCCTGCATCTATGTCTATGCCCGGCCCTTCACTGCCCTCCCC | |
| ACAGACACTGCCATCTCTGTCACCTTCACTGTCATCTCCCCTTTGCTCAATCCTATAATTTA | |
| CACGCTGAGGAATCAGGAAATGAAGTTIGGCCATGAGGAAACTGAAGAGACGGCTAGGAC | |
| AATCAGAAAGGATTTTAATTCAATAA. | |
| AOLFR238 sequences: | |
| MAPEMTTRVTEFILTGVSSCPELQIPLFLVFLVLTVLTMAGNLGIITLTSVDSRIQTPMTTFLRHL | (SEQ ID NO:445) |
| AIINLGNSTVIAPKMLMNFLVKKKTTSFTECATQLGGFLFFIVSEVMMLAVMATDRTVAICNP | |
| LLYMVVVSRRLCLLLVSLTTLTGFSTAIVVSPCIFSVSTCSSNITTNHFTCDIAPLLALSCSDTYIPE | |
| TIVFISAATNLFFSMITVLVSTTNIVLSILRIRSPEGRLKKAFSTCASHMIAVTVFTGTMLFMTLQP | |
| QTNHSLDTDKMASVFTTLVIPMLNPLITSLRNNDVNVALKKFMENPCTSFKSM. | |
| ATGGGTCCTGAAAATTTCACCAGGGTCACTGAGTTTATTCTCACAGGTGTCTCTAGCTGTC | (SEQ ID NO:446) |
| CAGAGCTCCAGATTCCCCTCTTCCTGGTCTTCCTAGTGCTCTATGTGCTGACCATGGCAGG | |
| GAACCTGGGCATCATCACCCTCACCAGTGTTGACTCTCGACTTCAAACCCCCATGTACTTTT | |
| TCCTGAGACATCTAGCTATCATCAATCTTGGCAACTCTACTGTCATTGCCGCTAAAATGCTG | |
| ATGAACTTTTTAGTAAAGAAGAAAACTACCTCATTCTATGAATGTGCCACCCAACTGGGAG | |
| GGTTCTTGTTCTTTATTGTATCGGAGGTAATGATGCTGGCTGTGATGGCCTATGACCGCTA | |
| TGTGGCCATTTGTAACCCTCTGCTCTACATGGTGGTGGTGTCTCGGCGGCTCTGCCTCCTGC | |
| TGGTGTCCCTCACGTACCTCTATGGCTTTTCTACAGCTATTGTGGTTTCACCTTGTATATTC | |
| TCTGTGTCTTATTGCTCTTCTAATATAATCAATCATTTTTACTGTGATATTGCACCTCTGTT | |
| AGCATTATCTTGCTCTGATACTTACATACGAGAAACAATAGTCTTTATATCTGCAGCAACA | |
| AATTTGTTTTTTTCGATGATTACAGTTCTAGTATCTTATTTCAATATTGTTTTGTCCATTCTA | |
| AGGATACGTTCACCAGAAGGAAGGAAAAAAGCCTTTTCCACCTGGGCTTCGCATATGATA | |
| GCAGTCACGGTTTTCTATGGGACAATGCTATTTATGTATTTGCAGCCCCAAACCAACCACT | |
| CACTGGATACTGATAAGATGGCTTCTGTGTTTTTACACATTGGTGATTCCTATGCTGAATCC | |
| CTTGATCTACAGCCTGAGGAATAATGATGTAAATGTTGCCTTAAAGAAATTCATGGAAAAT | |
| CCATGTTACTCCTTTAAATCAATGTAA. | |
| AOLFR239 sequences: | |
| MDPQNTSLVSEFVLHGLCTSRIILQNIFFFIFFFGVTVAIMLGNLLILVTVISDPCLHSSPMTFLLG | (SEQ ID NO:447) |
| NLAFLDMWLASFATPKMIRDFLSDQKLISFGGCMAQIFFLHFTGGAEMVLLVSMATDRTVAIC | |
| KPLHTMTLMSWQTCIRLVLASWVVGFVHSISQVAFTVNLPTCGPNEVDSFFCDLPINIKLACM | |
| DTYVLGIIMISDSGLLSLSCFLLLLISTTVILLAIRQRAAGSTSKALSTCSAHIMVVTLFFGPCIFV | |
| YVRPFSRFSVDKLLSVFTTIFTPLLNPIITTLRNEEMKAAMKKLQNRRVTFQ. | |
| ATGGACCCACAGAACTATTCCTTGGTGTCAGAATTTGTGTTGCATGGACTCTGCACTTCAG | (SEQ ID NO:448) |
| GACATCTTCAAAATTTTTCTTTTATATTTTCTTTGGGGTCTATGTGGCCATTATGCTGGGT | |
| AACCTTCTCATTTTGGTCACTGTAATTTCTGATCCCTGCCTGCACTCCTCCCCTATGTACTT | |
| CCTGCTGGGGAACCTAGCTTTCCTGGACATGTGGCTGGCCTCATTTGCGACTCCCAAGATG | |
| ATCAGGGATTTCCTTAGTGATCAAAAACTCATCTCCTTTGGAGGATGTATGGCTCAAATCT | |
| TCTTCTTGCACTTTACTGGTGGGGCTGAGATGGTGCTCCTGGTTTCCATGGCCTATGACAG | |
| ATATGTGGCCATATGCAAACCCTTGCATTACATGACTTTGATGAGITGGCAGACTTGCATC | |
| AGGCTGGTGCTGGCTTCATGGGTCGTTGGATTTGTGCACTCCATCAGTCAAGTGGCTTTTCA | |
| CTGTAAATTTGCCTTACTGTGGCCCCAATGAGGTAGACAGCTTCTTCTGTGACCTCCCTCTG | |
| GTGATCAAACTTGCCTGCATGGACACCTATGTCTTGGGTATAATTATGATCTCAGACAGTG | |
| GGTTTGCTTTCCTTGAGCTGTTTTTCTGCTCCTCCTGATCTGCTACACCGTGATCCTCCTCGCT | |
| ATCAGACAGCGTGCTGCCGGTAGCACATCCAAAGCACTCTCCACTTGCTCTGCAGATATCA | |
| TGGTAGTGACGCTGTTCTTTGGCCCTTGCATTTTTGTTTATGTGCGGCCTTTCAGTAGGTTC | |
| TCTGTGGACAAGCTGCTGTCTGTGTTTTATACCATTTTTACTCCACTCCTGAACCCCATTAT | |
| CTACACATTGAGAAATGAGGAGATGAAAGCAGCTATGAAGAAACTGCAAAACCGACGGGT | |
| GACTTTTCAATGA. | |
| AOLFR24G sequences: | |
| MAGENHTTLPEFLLLGFSDLKALQGPLFWVVLLVTLVTLLGNSLHLLTQVSPALHSPMTTFLR | (SEQ ID NO:449). |
| QLSVVELFTTTDIVPRTLANLGSPHPQAISFQGCAAQMTVFIVLGISECCLLTAMATDRTVAIC | |
| QPLRTSTLLSPRACLAMVGSSWLTGHTATTHASLIFSLPFRSHPIIPHFLCDILPVLRLASAGKHR | |
| SEISVMTATIVFIMIPFSLIVTSTRILGAILAMASTQSRRKKTFSTCSSHLLVVSLFFGTASITTRPQ | |
| AGSSVTTDRVLSLFTTVITPMLNPIITTLRNKDVRRALRHLVKRQRPSP. | |
| ATGGCTGGGGAAAACCATACTACACTGCCTGAATTCCTCCTTCTGGGATTCTCTGACCTCA | (SEQ ID NO:450) |
| AGGCCCTGCAGGGCCCCCTGTTCTGGGTGGTGCTTCTGGTCTACCTGGTCACCTTGCTGGG | |
| TAACTCCCTGATCATCCTCCTCACACAGGTCAGCCCTGCCCTGCACTCCCCCATGTACTTCT | |
| TCCTGCGCCAACTCTCAGTGGTGGAGCTCTTCTAGACCACTGACATCGTGGCCAGGACCCT | |
| GGCCAATCTGGGCTCCCCGCATCCCCAGGCCATCTCTETCCAGGGCTGTGCAGCCCAGATG | |
| TACGTCTTCATTGTCCTGGGGATCTCGGAGTGCTGCCTGCTCACGGCCATGGCCTATGACC | |
| GATATGTTGCCATCTGCCAGCCCCTACGCTATTCCACCCTCTTGAGCCCACGGGCCTGCTT | |
| GGCCATGGTGGGGTCCTCCTGGCTCACAGGCATCATCACGGCCACCACCCATGCCTCCCTC | |
| ATCTTCTCTCTACCTTTTCGCAGCCACCCGATCATCCCGCAGTTTCTCTGTGAGATCCTGCC | |
| AGTACTGAGGCTGGCAAGTGCTGGGAAGCACAGGAGCGAGATCTCCGTGATGACAGCCAC | |
| CATAGTCTTCATTATGATCCCCTTCTCTCTGATTGTCACCTCTTACATCCGCATCCTGGGTG | |
| CCATCCTAGCAATGGCCTCCACCCAGAGCCGCCGCAAGGTCTTCTCCACCTGCTCCTCCCA | |
| TCTGCTCGTGGTCTCTCTCTTCTTTGGAACAGCCAGCATCACCTACATCCGGCCGCAGGCA | |
| GGCTCCTCTGTTACCACAGACCGCGTCCTCAGTCTCTTCTACACAGTCATCACACCCATGCT | |
| CAACCCCATCATCTACACCCTTCGGAACAAGGACGTGAGGAGGGCCCTGCGACACTTGGT | |
| GAAGAGGCAGCGCCCCTCACCCTGA. | |
| AOLFR241 sequences: | |
| MPQILIFTTLNMFTTFPPLQILAENLTMVTEFLLLGFSSLGEIQLAIFVVFLFLTLVILSGNVTIIS | (SEQ ID NO:451) |
| VIHLDKSLHTPMTTFLGILSTSETFTTFVILPKMLINLLSVARTISFNCCALQMFFFLGFATTNCLL | |
| LGVMGTDRTAAICHPLHTPTLMSWQVCGKLAAACAIGGFLASLTVVNLVFSLPFCSANKVNH | |
| YFCDISAVILLACTNTDVNEFVIFICGVLVLVTVPFLFICVSTLCILRTILTPSAEGRRKAFSTCAS | |
| HLSVVIVHTGCASFITLRPTANTVSNKDRLVTVTTTIVTPLLNPMVTSLRNKDVQLAIRKVLG | |
| KKGSLKLYN. | |
| ATGCCCCAAATTCTTATATTCACATACCTGAATATGTTTTACTTCTTTCCCCCTTTGCAGAT | |
| CTTGGCAGAAAACCTCACCATGGTCAGCGAATTCCTGTTGGTGGGTTTTTCGAGCCTTGGT | |
| GAAATTCAGCTGGCCCTCTTTGTAGTTTTTCTTTTTCTGTATCTAGTCATTCTTAGTGGCAA | |
| TGTCACCATTATCAGTGTCATCCACCTGGATAAAAGGCTCCACACACCAATGTACTTCTTTCC | |
| TTGGCATTCTCTCAACATCTGAGACCTTCTACACCTTTGTCATTCTACCCAAGATGCTCATC | |
| AATCTACTTTCTGTGGCCAGGACAATCTCCTTCAACTGTTGTGCTCTTCAAATGTTCTTCTT | |
| CCTTGGTTTTGCCATTACCAACTGCCTGCTATTGGGTGTGATGGGTTATGATCGCTATGCTG | |
| GCATTTGTCACCCTCTGCATTACCCCACTCTTATGAGCTGGCAGGTGTGTGGAAAACTGGC | |
| AGCTGCCTGTGCAATTGGTGGCTTCTTGGCCTCTCTTACAGTAGTAAATTTAGTTTTCAGCC | |
| TCCCTTTTTGTAGCGCCAACAAAGTCAATCATTACTTCTGTGACATCTCAGCAGTCATTCTT | |
| CTGGCTTGTACCAACACAGATGTTAACGAATTTGTGATATTCATTTGTGGAGTTCTTGTAC | |
| TTGTGGTTCCGTTTCTGTTTATCTGTGTTTCTTATCTCTGCATTCTGAGGACTATCCTGAAG | |
| ATTCCCTCAGCTGAGGGCAGACGGAAAGCGTTTTCCACCTGCGCCTCTCACCTCAGTGTTG | |
| TTATTGTTCATTATGGCTGTGCTTCCTTCATCTACCTGAGGCCTACAGCAAACTATGTGTCC | |
| AACAAAGACAGGCTGGTGACGGTGACATACACGATTGTCACTCCATTACTAAACCCCATG | |
| GTTTATAGCCTCAGAAACAAGGATGTCCAACTTGGTATCAGAAAAGTGTTGGGCAAGAAA | |
| GGTTCTCTAAAACTATATAATTGA. | |
| AOLFR242 sequences: | |
| MNTTLFHPTSFLLLGIPGLESMHLWVGFPFFAVFLTAVLGNITILFVIQTDSSLHIIPMFTTLAILS | (SEQ ID NO:453) |
| SIDPGLSTSTIPKMLGTFWFTLREISFEGCLTQMFFIHLCTGMESAVLVAMATDCTVAICDPLCY | |
| TLVLTNKVVSVMALAIFLRPLVFVIPFVLFILRLPFCGHQIIPHTTGEHMGIARLSCASIRVNIIYG | |
| LCAISILVFDIIAIVISTVQILCAVFLLSSHDARIKAFSTCGSHVCVMLTFTMPAFFSFMTHRFGR | |
| NIPHFIHILLANFTVVIPPALNSVITGVRTKQIRAQVLKMFFNK. | |
| ATGAATACCACTCTATTTCATCCTTACTCTTTCCTTCTTCTGGGAATTCCTGGGCTGGAAAG | (SEQ ID NO:454) |
| TATGCATCTCTGGGTTGGTTTTCCTTTCTTTGCTGTGTTCCTGACAGCTGTGCTTTGGGAATA | |
| TCACCATCCTTTTTGTGATTCAGACTGACAGTAGTCTCCATCATCCCATGTTCTACTTCCTG | |
| GCCATTCTGTCATCTATTGACCCGGGCCTGTCTACATCCACCATCCCTAAAATGCTTGGCAC | |
| CTTCTGGTTTACCCTGAGAGAAATCTCCTTTGAAGGATGCCTTACCCAGATGTTCTTCATCC | |
| ACCTGTGCACTGGCATGGAATCAGCTGTGCTTGTGGCCATGGCCTATGATTGCTATGTGGC | |
| CATCTGTGACCCTCTTTGCTACACGTTGGTGCTGACAAACAAGGTGGTGTCAGTTATGGCA | |
| CTGGCCATCTTTCTGAGACCCTTAGTCTTTGTCATACCCTTTGTPTCTATTTATCCTAAGGCT | |
| TCCATTTTGTGGACACCAAATTATTCCTCATACTTATGGTGAGCACATGGGCATTGCCCGC | |
| CTGTCTTGTGCCAGCATCAGGGTTAACATCATCTATGGCTTATGTGCCATCTCTATCCTGGT | |
| CTTTGACATCATAGCAATTGTCATTTCCTATGTACAGATCCTTTTGTGCTGTATTTCTACTCT | |
| CTTCACATGATGCACGACTCAAGGCATTCAGCACCTGTGGCTCTCATGTGTGTGTCATGTT | |
| GACTTTCTATATGCCTGCATTTTTCTCATTCATGACCCATAGGTTTGGTCGGAATATACCTC | |
| ACTTTATCCACATICTTCTGGCTAATTCTATGTAGTCATTCCACCTGCTCTCAACTCTGTA | |
| ATTTATGGTGTCAGAACCAAACAGATTAGAGCACAAGTGCTGAAAATGTTTTTCAATAAAT | |
| AA. | |
| AOLFR243 sequences: | |
| MEQVNKTVVREFVVLGFSSLARIQQLLFVIFLLLTLETLGTNAIIISTIVLDRALHTPMYFFLAIL | (SEQ ID NO:455) |
| SCSEICTTFVIVPKMLVDLLSQKKTISFLGCAIQMFSFLFFGSSHSFLLAAMGTDRTMAICNPLR | |
| YSVLMGHGVCMGLMAAACAGGFTVSLVTFTSLVIFHLPFHSSNQLHHFFCDISPVLKLASQHSGF | |
| SQLVIFMLGVFALVIPLLLILVSTRIISAILTPSSVGRTKTFSTCASIILIVVTVHTSCASFITLRPK | |
| TNYTSSQDTLISVSTTILTPLFNPMITSLRNKEFKSALRRTIGQTFTPLS. | |
| ATGGAGCAAGTCAATAAGACTGTGGTGAGAGAGTTCGTCGTCCTCGGCTTCTCATCCCTGG | (SEQ ID NO:456) |
| CCAGGCTGCAGCAGCTGCTCTTTGTTATCTTCCTGCTCCTCTACCTGTTCACTCTGGGCACC | |
| AATGCAATCATCATTTCCACCATTGTGCTGGACAGAGCCCTTCATACTCCCATGTACTTCTT | |
| CCTTGCCATCCTTTCTTGCTCTGAGATTTGCTATACCTTTGTCATTGTACCCAAGATGCTGG | |
| TTGACCTGCTGTCCCAGAAGAAGACCATTTCTTTCCTGGGCTGTGCCATCCAAATGTTTTCC | |
| TTCCTCTTCTTTGGCTCCTCTCACTCCTTCCTGCTGGCAGCCATGGGCTATGATCGCTATAT | |
| GGCCATCTGTAACCCACTGCGCTACTCAGTGCTCATGGGACATGGGGTGTGTATGGGACTA | |
| ATGGCTGCTGCCTGTGCCTGTGGCTTCACTGTCTCCCTGGTCACCACCTCCCTAGTATTTCA | |
| TCTGCCCTTCCACTCCTCCAACCAGCTCCATCACTTCTTCTGTGACATCTCCCCTGTCCTTA | |
| AACTGGCATCTCAGCACTCCGGCTTCAGTCAGCTGGTCATATTCATGCTTGGTGTATTTGC | |
| CTTGGTCATTCCTCTGCTACTTATCCTAGTCTCCTACATCCGCATCATCTCTGCCATTCTAA | |
| AAATCCCTTCCTCCGTTGGAAGATACAAGACCTTCTCCACCTGTGCCTCCCATCTCATTGTG | |
| GTAACTGTTCACTACAGTTGTGCCTCTTTCATCTACTTAAGGCCCAAGACTAATTAGAGTTC | |
| AAGCCAAGACACCCTAATATCTGTGTCATACACCATCCTTACCCCATTGTTCAATCCAATG | |
| ATTTATAGTCTGAGAAATAAGGAATTCAAATCAGCCCTACGAAGAACAATCGGCCAAACT | |
| TTCTATCCTCITAGTTAA. | |
| AOLFR244 sequences: | |
| MWQETTTLNVFFPLLKVCCLTTSHVVILLPWECTHLIWTLPTGTTVGSMEETNTSSTDFTF | (SEQ ID NO:457) |
| MGLFNRKETSGLIFAIISIIFFTALMANGVMIFLIQTDLRLHTPMTFLLSHLSLIDMMTSTTTPKM | |
| LVNTLLDQRTISFVGCTAQHFLTLTLVGAEFFLLGLMATDRTVAICNPLRTPVLMSRRVCWMI | |
| lAGSWFGGSLDGFLLTPITMSFPFCNSREIINHFFCEAPAVLKLACADTALTETVMTVCCVLMLL | |
| IPFSVVLASTARILTTVQCMSSVEGRKKAFATCSSHMTVVSLFTGAAMTTTMLPHSTIKPAQ | |
| DKVLSVFTTILTPMLNPLITSLRM(DVTGALKRALGRFKGPQRVSGGVF. | |
| ATGTGGCAAGAATACTATTTTTTAAATGTTTTGTTCCCACTTTTAAAAGTTTGCTGCCTAAC | (SEQ ID NO:458) |
| AATTAATTCACATGTTGTTATTTTACTGCCCTGGGAATGCTATCATCTTATTTGGAAGATAT | |
| TACCTTATATCGGCAGAACTGTAGGATCAATGGAAGAGTAGAACACATCCTCTACAGACTT | |
| CACTTTCATGGGGCTGTTCAACAGAAAGGAAACCTCAGGTCTTATTTTTGCCATCATCTCT | |
| ATCATCTTCTTCACCGCACTGATGGCCAATGGGGTTATGATCTTCCTGATCCAAACAGATT | |
| TGCGCCTTCATACACCCATGTACTTCCTCCTCAGCCACCTTTCCTTAATTGACATGATGTAT | |
| ATTTCCACTATTGTGCCTAAGATGCTGGTTAATTACCTGCTGGATCAAAGGACCATTTCCTT | |
| TGTGGGGTGCACAGCTCAACACTTCCTCTACCTTACCCTTGTGGGAGCTGAATTCTTCCTG | |
| GTGGGCCTCATGGCCTATGACCGCTATGTGGCCATTTGCAACCCTCTGAGATACCCTGTCC | |
| TCATGAGCCGCCGGGTCTGTTGGATGATTATAGCAGGTTCCTGGTTTGGGGGCTCTTTGGA | |
| TGGGTTCCTCCTAACCCCCATCACCATGAGCTTTCCCTTCTGCAATTCCCGGGAGATTAACC | |
| ACTTCTTCTGTGAGGCACCAGCAGTCCTGAAGTTGGCATGTGCAGACACAGCCCTCTACGA | |
| GACAGTGATGTATGTGTGCTGTGTTTTGATGCTGCTGATTCCTTTCTCTGTAGTCGTTGCTT | |
| CCTATGCCCGAATCCTGACTACAGTTCAGTGCATGAGCTCAGTGGAGGGCAGGAAGAAGG | |
| CATTTGCCACTTGCTCATCCCACATGACTGTGGTGTCCTVTGTTCTACGGGGCTGCCATGTAC | |
| ACCTACATGCTGCCACATTCTTACCACAAGCCAGCCCAGGACAAAGTCCTCTCTGTGTTTT | |
| ACACCATTCTCACACGCATGCTGAACCCCCTCATCTACAGCCTTAGAAACAAGGATGTGAC | |
| TGGAGCTCTGAAGAGGGCCTTGGGGAGGTTCAAGGGTCCTCAAAGGGTGTCAGGAGGTGT | |
| CTTTTGA. | |
| AOLFR245 sequences: | |
| MDLKNGSLVTEFILLGFFGRWELQIFFFVTFSLITGATVMGNILIMVTVTCRSTLHSPLTTLLGN | (SEQ ID NO:459) |
| LSFLDMCLSTATTPKMIIDLLTDHKTISVWGCVTQMFFMHFFGGAEMTLLIIMMDRTVAICKLP | |
| LHYRTIMSIHKLLKGFAILSWIIGFLHSISQIVLTMNLPFCGHNVTNNIFCDLPLVIKLACIETYTLE | |
| LFVIADSGLLSFTCFILLLVSTVILVSVPKKSSHGLSKALSTLSAHIIVVTLFFGPCIHTVWPFSSL | |
| ASNKTLAVFTTVITPLLNPSITTLRNKLKMQEAIRKLRFQTVSSAQNF. | |
| ATGGATCTTAAAAATGGATCTGTAGTGACCGAGTTTATTTTACTAGGATTTTTTGGACGAT | (SEQ ID NO:460) |
| GGGAACTTCAAATTTTCTTCTTTGTGACATTTTCCCTGATCTACGGTGCTACTGTGATGGGA | |
| AACATTCTCATTATGGTCACAGTGACATGTAGGTCAACCCTTCATTGTCCCTTGTACTTTCT | |
| CCTTGGAAATCTCTCTTTTTTGGACATGTGTCTCTCCACTGCCACAACACCCAAGATGATCA | |
| TAGATTTGGTCACTGACCACAAGACCATCTCTGTGTGGGGCTGCGTGACCCAGATGTTCTT | |
| CATGCACTTCTTTGGGGGTGCTGAGATGACTCTTCTGATAATCATGGCCTTTGACAGGTAT | |
| GTAGCCATATGTAAACCCCTGCACTATAGGACAATCATGAGCCACAAGCTGCTAAAGGGG | |
| TTTGCGATACTTTCATGGATAATTGGTTTTTTACACTCCATAAGCCAGATAGTTTTAACAAT | |
| GAACTTGGCTTTCTGTGGCCACAATGTCATAAACAACATATTTTGTGATCTTCCCCTTGTGA | |
| TCAAGCTTGCTTGCATTGAAACATACACCCTGGAATTATTTGTCATTGCTGACAGCGGGCT | |
| GCTCTCTTTCACCTGTTTCATCCTCTTGCTTGTTTCTTACATTGTCATCCTGGTCAGTGTACC | |
| AAAAAAATCATCACATGGGCTCTCCAAGGCGCTGTCCACATTGTCTGCCCACATGATTGTG | |
| GTCACTCTGTTCTTTGGACCTTGTATTTTTATCTATGTTTGGCCATTCAGTAGTTTGGCAAG | |
| CAATAAAACTCTTGCCGTATTTTATACAGTTATCACACGCTTACTGAATCCGAGTATTTATA | |
| CCCTGAGAAATAAGAAAATGCAAGAGGCCATAAGAAAATTACGGTTCCAATATGTTAGTT | |
| CTGCACAGAATTTCTAG. | |
| AOLFR246 sequences: | |
| MSPENQSSVSEFLLLGLPIRPEQQAVFFTLFLGMTLTTVLGNLLIMLLIQLDSHLHTPMTTFLSH | (SEQ ID NO:461) |
| LALTDISFSSVTVPKMLMDMRTKTKSILTEECISQMTTFIFFTDLDSFLITSMATDRTVAICHPL | |
| HYTVIMREELCVFLVAVSWILSCASSLSHTLLLTRLSFCAANTIPHVFCDLAALLKLSCSDIFLNE | |
| LVMFTVGVVVITLPFMCILVSTGTGATILRVPSTKGIHKALSTCGSHLSVVSLTTGSIFGQYLF | |
| PTVSSSIDKDVIVALMTTVVTPMLNPFITSLRNRDMKEALGKLFSRATFFSW. | |
| ATGAGCCCTGAGAACCAGAGGAGCGTGTCCGAGTTCCTCCTTCTGGGCCTCCCCATCCGGC | (SEQ ID NO:462) |
| CAGAGCAGCAGGCTGTGTTCTTCACCCTGTTCCTGGGCATGTACCTGACCACGGTGCTGGG | |
| GAACCTGCTCATCATGCTGCTCATCCAGCTGGACTCTCACCTTCACACCCCCATGTACTTCT | |
| TCCTCAGCCACTTGGGTCTCACTGACATCTCCTTTTCATCTGTCACTGTCCCTAAGATGCTG | |
| ATGGAGATGCGGACTAAGTACAAATCGATCCTCTATGAGGAATGCATTTCTCAGATGTATT | |
| TTTTTATAtTTTTTACTGACCTGGACAGGTTCCTTATTACATCAATGGCATATGACCGATAT | |
| GTTGCCATATGTCACCCTCTCCACTACACTGTCATCATGAGGGAAGAGCTCTGTGTCTTCTT | |
| AGTGGCTGTATCTPGGATTGTGTCTTGTGCCAGCTCCCTCTCTCACACCCTTCTCCTGACCC | |
| GGCTGTCTTTCTGTGCTGCGAACACCATGCCCCATGTCTTCTGTGACCTTGCTGCCCTGCTC | |
| AAGCTGTCCTGCTCAGATATCTTCCTCAATGAGCTGGTCATGTTCACAGTAGGGGTGGTGG | |
| TCATTACCCTGCCATTCATGTGTATCCTGGTATCATATGGCTACATTGGGGCCACCATCCTG | |
| AGGGTCCCTTCAACCAAAGGGATCCACAAAGCATGTGTCCACATGTGGCTCCCATCTCTCTG | |
| TGGTGTCTCTCTATTATGGGTCAATATTTGGCCAGTACCTTTTCCCGACTGTAAGCAGTTCT | |
| ATTGAGAAGGATGTCATTGTGGCTCTCATGTACACGGTGGTCACACCCATGTTGAACCCCT | |
| TTATCTACAGCCTTAGGAACAGGGACATGAAAGARGCCCTTGGGAAACTCTTCAGTAGAG | |
| CAACATTTTTCTCCTTGGTGACATCTGACTTTTTAAAAAATTAG. | |
| AOLFR247 sequences: | |
| MGQHNLTVLTEFILMELTRRIPELQIPLFGVFLVITLITVVGNLTMIILTKLDSHLHTPMTTSIRHL | (SEQ ID NO:463) |
| ASVDLGNSTVICPKVLANFVVDRNTISTTACAAQLAFFLMFIISEFFILSAMATDRTVAICNPLL | |
| YYVIMSQRLCHVLVGTQTLTSTFQALMFTITFTLTFCGSNVISHFTCDDVPLLPMLCSNAQEIE | |
| LLSILFSVFNLISSFLIVLVSTMLILLAICQMIISAEGRKKAFSTCGSHLTVVVVFTGSLLFMYMQ | |
| PNSTHFFDTDKMASVFTTLVIPMLNPLITSLRNEEVKNAFTKLFEN. | |
| ATGGGCCAACACAATCTAACAGTGCTAACTGAATTCATTCTGATGGAACTCACAAGGCGGC | (SEQ ID NO:464) |
| CTGAGCTGCAGATTCCCCTTTTTTGGAGTCTTTCCTCGTCATCTACCTAATCACAGTGGTGGGC | |
| AACCTAACTATGATCATTTTGAGCAAACTGGACTCCCACTTACATACACCTATGTACTTTTTC | |
| TATCAGACATTTGGCTTCTGTTGATCTTGGTAATTCTACTGTCATTTGTCCCAAGGTGCTGG | |
| CAAATTTTGTTGTGGATCGAAATACTATTTCCTATTATGCATGTGCTGCACAGCTGGCATTC | |
| TTCCTTATGTTCATTATCAGTGAATTTTTCATCCTGTCAGCCATGGCCTATGACCGCTATGT | |
| GGCGATTTGTAACCCTCTGCTCTATTATGTTATTATGTCTCAGCGACTGTGTCATGTACTGG | |
| TGGGCATTCAATATCTCTACAGCACATTTCAGGCTCTGATGTTCACTATTAAGATTTTTACA | |
| TTGACCTTCTGTGGCTCTAATGTCATCAGTCATTTTPACTGTGATGATGTTCCTTTGCTACC | |
| TATGCTTTGCTCAAATGCACAGGAAATAGAATTGTTGAGCATACTATTTTCTGTATTTAATT | |
| TGATCTCCTCCTTTCTGATAGTCTTAGTGTCCTACATGTTGATTTTGTTAGCTATATGTCAA | |
| ATGCATTCTGCAGAGGGCAGGAAAAAGGCTIITCTCCACATGTGGTTCCCATTTGACAGTGG | |
| TGGTTGTGTTCTATGGGTCTCTACTCTTCATGTACATGCAGCCCAATTCCACTCACTTCTTT | |
| GATACTGATAAAATGGCTTCTGTGTTTTACACTTTAGTAATCCCCATGCTTAACCCTTTGAT | |
| TTACAGCTTAAGAAACGAAGAGGTGAAAAATGCCTTCTATAAGCTCTTTGAGAATTGA. | |
| AOLFR248 sequences: | |
| MPCMI2CALPTGGLLPHPQHTMMEIANVSSPEVFVLLGFSTRPSLETVLFIVVLSFTMVSILGNGI | (SEQ ID NO:465) |
| IILVSLITDVHLHTPMTTFLANLPFLDMSFTTSIVPQLLANLWGPQKTISTGGCVVQFTSHWLG | |
| ATECVLLATMSTDRTAAICRPLHTTVIMHPQLCLGLALASWLGGLTTSMVGSTLTMLLPLCG | |
| NNCIDHFFCEMPLIMQLACVDTSLNEMEMTLASFVFVVLPLGLILVSTGHIARAVLTRSAEGR | |
| RKAFNTCSSHVAVVSLFTGSIIFMTLQPAKSTSHEQGKFIALFTTVVTPALNPLITTLRNTEVKS | |
| ALRHMVLENCCGSAGKLAQI. | |
| ATGCCCTGTATGCCCTGTGCTCTTCCCACAGGTGGCCTTTTTGCCCCACCCCCAGCATACAAT | (SEQ ID NO:466) |
| GATGGAAATAGCCAATGTGAGTTCTCCAGAAGTCTTTGTCCTCCTGGGCTTCTCCACACGA | |
| CCCTCACTAGAAACTGTCCTCTTCATAGTTGTCTTGAGTTTTTACATGGTATCGATCTTGGG | |
| CAATGGCATCATCATTCTGGTCTCCCATACAGATGTGCACCTCCACACACCTATGTACTTGT | |
| TTCTTGCCAACCTCCCCTTCCTGGACATGAGGTTCACCACGAGCATTGTCCCACAGCTCCTG | |
| GCTAACCTCTGGGGACCACAGAAAACCATAAGCTATGGAGGGTGTGTGGTCCAGTTCTAT | |
| ATCTCCCATTGGCTGGGGGCAACCGAGTGTGTCCTGCTGGCCACCATGTCCTATGACCGCT | |
| ACGCTGCCATCTGCAGGCCACTCCATTACACTGTCATTATGCATCCACAGCTTTGCCTTGG | |
| GCTAGCTTTGGCCTCCTGGCTGGGGGGTCTGACCACGAGCATGGTGGGCTCCACGCTCACC | |
| ATGCTCCTACCGCTGTGTGGGAACAATTGCATCGACCACTTCTTTTTGCGAGATGCCCCTCA | |
| TTATGCAAGTGGCTTGTGTGGATACCAGCCTCAATGAGATGGAGATGTACCTGGCCAGCTT | |
| TGTGTTTGTTGTCCTGCCTCTGGGGCTCATCCTGGTCTCTTACGGCCACATTGCCCGGGCCG | |
| TGTTGAAGATCAGGTCAGCAGAAGGGCGGAGAAAGGCATTCAACACCTGTTCTTCCCACG | |
| TGGCTGTGGTGTCTCTGTTTTACGGGAGCATCATCTTCATGTATCTCCAGCCAGCCAAGAG | |
| CACCTCCCATGAGCAGGGCAAGTTCATAGCTCTGTTCTACACCGTAGTCACTCCTGCGCTG | |
| AACCCACTTATITTACACCCTGAGGAACACGGAGGTGAAGAGCGCCCTCCGGCACATGGTA | |
| TTAGAGAACTGCTGTGGCTCTGCAGGCAAGCTGGCGCAAATTTAG. | |
| AOLFR249 sequences: | |
| MKSQIEKSDLKTRAILLQKVTRMFLLFWVLLLVLSRLLVVMGRGNSTEVTEFHLLGFGVQHEF | (SEQ ID NO:467) |
| QHVLFIVLLLITVTSLIGNIGMILLIKTDSRLQTPMTTFPQHLAFVDICTTSATTPKMLQSFTEEN | |
| NLITFRGCVIQFLVTATFATSDCTLLAIMAMDCTVAICKPLRTPMIMSQTVTQLVAGSTIGSI | |
| NASVHTGFTFSLSFCKSNKTh4HFFCDGLPILALSCSNIDINIILDVVFVGFDLMFTELVIIFSYIYIM | |
| VTILKMSSTAGRKKSFSTCASHLTAVTIFTGTLSTMTLQPQSNNSQENMKVASIFTGTVIPMLN | |
| PLIYSLRNKEGK. | |
| ATGAAAAGTCAAATTGAAAAAAGTGACTTAAAATATAGAGCCATTTTATTGCAAAAAGTC | (SEQ ID NO:468) |
| ACAAGGATGTTCCTGCTTTTCTGGGTCCTTCTCTTGGTCCTTTCTAGACTTTTGGTAGTCAT | |
| GGGTCGAGGAAACAGCACTGAAGTGACTGAATTCCATCTTCTGGGATTTGGTGTCCAACAC | |
| GAATTTCAGCATGTCCTTTTCATTGTACTTCTTCTTATCTATGTGACCTCCCTGATAGGAAA | |
| TATTGGAATGATCTTACTCATCAAGACCGATTCCAGACTTCKAACACCCATGTACTTTTTTC | |
| CACAACATTTGGCTTTTGTTGATATCTGTTATACTTCTGCTATCACTCCCAAGATGCTCCAA | |
| AGCTTCACAGAAGAAAATAATTTGATAACATTTCGGGGCTGTGTGATACAATTCTTAGTTT | |
| ATGCAACATTTGCAACCAGTGACTGTTACCTCCTAGCTATTATGGCAATGGATTGTTATGT | |
| TGCGATCTGTAAGCCCCTTCGCTATCCCATGATCATGTCCCAAACAGTCTACATCCAACTCG | |
| TAGCTGGCTCATATATTATAGGCTGAATAAATGCCTCTGTACATACAGGTTTTACATTTTCA | |
| CTGTCCTTCTGCAAGTCTAATAAAATCAATCACTTTTTCTGTGATGGTCTCCCAATTCTTGC | |
| CCTTTCATGCTCCAACATTGACATCAACATCATTCTAGATGTTGTCTTTGTGGGATTTGACT | |
| TGATGTTCACTGAGTTGGTCATCATCTTTTCGTACATGTACATTATGGTGACCATCCTGAAG | |
| ATGTCTTCTACTGCTGGGAGGAAAAAATCCTTCTCCACATGTGCCTCCCACCTGACAGCAG | |
| TAACCATTTTCTATGGGACACTCTCTTACATGTACTTACAGCCTCAGTCTAATAATTCTCAG | |
| GAGAATATGAAAGTAGCCTCTATATTTTATGGCACTGTTATTCCCATGTTGAATCCTTTAAT | |
| CTATAGCTTGAGAAATAAGGAAGGAAAATAA. | |
| AOLFR25G sequences: | |
| MENQSSISEFFLRGISAPPEQQQSLFGIFLCMTLVTLTGNLLIILAIGSDLHLHTPMTTFLANLSFV | (SEQ ID NO:469) |
| DMGLTSSTVTKMLVNIQTRHHTISTTGCLTQMTTFLMFGDLDSFFLAAMATDRTVAICHPLCY | |
| STVMRPQVCALMLALCWVLTNIVALTHTFLMARLSFCVTGEIAHFFCDITPVLKLSCSDTHINE | |
| MMVFVLGGTVLIVPFLCIVTSTHIVPAILRVRTRGGVGKAFSTCSSHLCVVCVFTGTLFSAYLC | |
| PPSIASEEKDIAAAAMTTIVTPMLNPFITSLRNKDMKGALKRLFSHRSIVSS. | |
| ATGGAAAACCAATCGAGCATTTCTGAATTTTTCCTCCGAGGAATATCAGCGCCTCCAGAGC | (SEQ ID NO:470). |
| AACAGCAGTCCCTCTTCGGAATTTTCCTGTGTATGTATCTTGTCACCTTGACTGGGAACCTG | |
| CTCATCATCCTGGCCATTGGCTCTGACCTGCACCTCCACACCCCCATGTACTTTTTCTTGGC | |
| CAACGTGTGTTTTGTTGACATGGGTTTAACGTCCTCCACAGTTAGCAAGATGCTGGTGAAT | |
| ATACAGACTCGGCATCACACCATCTCCTATACGGGTTGCCTCACGCAAATGTATTTCTTTCT | |
| GATGTTTGGTGATCTAGACAGCTTCTTCCTGGCTGCCATGGCGTATGACCGCTATGTGGCC | |
| ATTTGCCACCCCCTCTGCTACTCCACAGTCATGAGGCCCCAAGTCTGTGCCCTAATGCTTGC | |
| ATTGTGCTGGGTCCTCACCAATATCGTTGCCCTGACTCACACGTTCCTCATGGCTCGGTTGT | |
| CCTTCTGTGTGACTGGGGAAATTGCTCACTTTTCTGTGACATCACTCCTGTCCTGAAGCTG | |
| TCATGTTCTGACACCCACATCAACGAGATGATGGTTTTTGTCTTGGGAGGCACCGTACTCA | |
| TGGTCCCCTTTTTATGCATTGTCAGCTCCTACATCCACATTGTGCCAGCTATCCTGAGGGTC | |
| CGAACCCGTGGTGGGGTGGGCAAGGCCTTTTCCACCTGCAGTTCCCACCTCTGCGTTGTTT | |
| GTGTGTTCTATGGGACCCTCTTCAGTGCCTACCTGTGTCCTCCCTCCATTGCCTCTGAAGAG | |
| AAGGACATTGCAGCAGCTGCAATGTACACCATAGTGACTCCCATGTTGAACCCCTTTATCT | |
| ATAGCCTAAGGAACAAGGACATGAAGGGGGCCCTAAAGAGGCTCTTCAGTCACAGGAGTA | |
| TTGTTTCCTCTTAG. | |
| AOLFR251 sequences: | |
| MEGNKTWITDITLPRFQVGPALEILLCGLFSAFTTLTLLGNGVIFGIICLDCKLHTPMTEFLSHLA | (SEQ ID NO:471) |
| IVDISTASNTVPKMLTNLMTh4QESTISFFPCIMQTFLTLAFAHVECLILVVMSTDRTADICHPLRY | |
| NILMSWRVCTVLAVASWVFSFLLALVPLVLILRIPFCGPHEINHFCEILSVLKLACADTWLNQV | |
| VIFAACVFILVGPLCLVLVSTLRILAAILRIQSGEGRRKAFSTCSSHLCVVGLFFGSAIVTTMAPK | |
| SRHPEEQQKVLSLFTSLFNPMLNPLITSLRTTAEVKGALRRALRKERLT. | |
| ATGGAAGGCAACAAGACATGGATCACAGACATCACCTTGCCGCGATTCCAGGTTGGTCCA | (SEQ ID NO:472) |
| GCACTGGAGATTCTCCTCTGTGGACTTTTCTCTGCCTTCTATACACTCACCCTGCTGGGGAA | |
| TGGGGTCATCTTTGGGATTATCTGCCTGGACTGTAAGCTTCACACAGCCATGTACTTCTTCC | |
| TCTCACACCTGGCCATTGTTGACATATCCTATGCTTCCAACTATGTCCCCAAGATGCTGACG | |
| AATCTTATGAACCAGGAAAGCACCATCTCCTTTTTTTCCATGCATAATGCAGACATTCTTGT | |
| ATTTGGCTTTTGCTCACGTAGAGTGTCTGATTTTGGTGGTGATGTCCTATGATCGCTATGCG | |
| GACATCTGCCACCCCTTACGTTACAATATCCTCATGAGCTGGAGAGTGTGCACTGTCCTGG | |
| CTGTGGCTTCCTGGGTGTTCAGGTTCCTCCTGGCTCTGGTCCCTTTAGTTCTCATCCTGAGG | |
| CTGCCCTTCTGCGGGCCTCATGAAATCAACCACTTGTGTGAAATCCTGTCTGTGCTCAAGTT | |
| GGCCTGTGCTGACACCTGGCTCAACCAGGTGGTCATCTTTGCAGCCTGCGTGTTCATCCTG | |
| GTGGGGCCACTCTGCCTGGTGGTGGTCTCCTACTTGCGCATCCTGGCCGCCATCTTGAGGA | |
| TGCAGTCTGGGGAGGGCCGCAGAAAGGCCTTCTCCACCTGCTCGTGCCACCTTTGCGTGGT | |
| GGGACTCTTCTTTGGCAGCGCCATTGTCACGTACATGGCCCCCAAGTCCCGCCATCCTGAG | |
| GAGCAGGAGAAAGTTCTTTCCCTGTTTTACAGCCTTTTCAATGCAATGCTGAACCCCCTGA | |
| TATATAGCCTAAGGAATGCAGAGGTCAAGGGCGCCCTGAGGAGGGCACTGAGGAAGGAG | |
| AGGCTGACGTGA. | |
| AOLFR252 sequences: | |
| MRLANQTLGGDFFLLGIFSQISHPGRLCLLIFSIFLMAVSWNITLILLTHTDSSLHTPMTTFTNQLSL | (SEQ ID NO:473) |
| IDLTTSVTVPKMLVNQLAKDKTISVLGCGTQMTTTLQLGGAECCLLAAMATDRTVAICHPLR | |
| YSVLMSHRVCLLLASGCWFVGSVDGFMLTPIAMSFPFCRSHEIQHFFCEVIPAVLKLSCSDTSLY | |
| KIFMTLCCVIMLLIPVTVISVSTTTILTIIIKMNSVEGRKKAFTTCSSHITVVSLFTGAAITNYML | |
| PSSYQTPEKDMMSSFFTTILTPVLNPIITSFRNKDVTRALKKMLSVQKPPY. | |
| ATGCGGCTGGCCAACCAGACCCTGGGTGGTGACTTTTTCCTGTTGGGAATCTTCAGCCAGA | (SEQ ID NO:474) |
| TCTCACACCCTGGCCGCCTGTGCTTGCTTATCTTCAGTATATTTTTGATGGCTGTGTCTTGG | |
| AATATTACATTGATACTTCTGATCCACATTGACTCCTCTCTGCATACTCCCATGTACTTCTT | |
| TATAAACCAGCTCTCACTCATAGACTTGACATATATTTCTGTCACTGTCCCCAAAATGCTG | |
| GTGAACCAGCTGGCCAAAGACAAGACCATCTCGGTCCTTGGGTGTGGCACCCAGATGTAC | |
| TTCTACCTGCAGTTGGGAGGTGCAGAGTGCTGCCTTCTAGCCGCCATGGCCTATGACCGCT | |
| ATGTGGCTATGTGCCATCCTCTCGGTTACTCTGTGCTCATGAGCCATAGGGTATGTCTCCTC | |
| CTGGCATCAGGCTGCTGGTTTGTGGGCTCAGTGGATGGCTTCATGCTCACTCCCATCGCCA | |
| TGAGCTTCCCCTTCTGCAGATCCCATGAGATTCAGCACTTCTTCTGTGAGGTCCCTGCTGTT | |
| TTGAAGCTCTCTTGCTCAGACACCTCACTTTACAAGATTTTCATGTACTTGTGCTGTGTCAT | |
| CATGCTCCTGATACCTGTGACGGTCATTTCAGTGTCTTACTACTATATCATCCTCACCATCC | |
| ATAAGATGAACTCAGTTGAGGGTCGGAAAAAGGCCTTCACCAGCTGCTCCTCCCACATTTAC | |
| AGTGGTCAGCCTCTTCTATGGAGCTGCTATTTACAACTACATGCTCCCCAGCTCCTACCAA | |
| ACTCCTGAGAAAGATATGATGTCATCCTTTTTCTACACTATCCTTACACCTGTCTTGAATCC | |
| TATCATTTTACAGTTTITCAGGAATAAGGATGTCACAAGGGCTTTGAAAAAAATGCTGAGCGT | |
| GCAGAAACCTCCATATTAA. | |
| AOLFR253 sequences: | |
| MTFFSSGGNCEPVMCSGNQTSQNQTASTDFTLTGLFAESKHAALLTTVTFLLFLMALTGNALL | (SEQ ID NO:475) |
| ILLIHSEPRIHTPMTTHSQLALMDLMTLCVTVPKMLVGQVTGDDTISPSGCGIQMFFHLTLAG | |
| AEVFLLAAMATDRTAAVCRIPLHTPLLMNQRVCQLLVSACWVLGMVDGLLLTPITMSFPFCQS | |
| RKILSFFGETPALLKLSCSDVSLTKMLTTLCCILMLLTPIMVISSSTTLILHLIHRMNSAAGRRKA | |
| LATCSSHMIIVLLLFGASFTTTMLRSSTHTAEQDMMVSAFTTIFTPVLNPLITSLRNKDVTRAL | |
| RSMMQSRMNQEK. | |
| ATGACTTTTTTTTTCCTCAGGGGGAAAGTGTGAGCCAGTCATGTGCTCAGGGAATCAGACTT | (SEQ ID NO:476) |
| CTCAGAATCAAACAGCAAGCACTGATTTCACCCTCACGGGACTCTTTGCTGAGAGCAAGCA | |
| TGCTGCCCTCCTCTACACCGTGACCTTCCTTCTTTTCTTGATGGCCCTCACTGGGAATGCCC | |
| TCCTCATCCTCCTCATCCACTCAGAGCCCCGCCTCGACACCCCCATGTACTTCTTCATCAGC | |
| CAGCTCGCGCTCATGGATCTCATGTACCTATGCGTGACTGTGCCCAAGATGCTTGTGGGCC | |
| AGGTCACTGGAGATGATACCATTTCCCCGTCAGGCTGTGGGATCCAGATGTTCTTCCACCT | |
| GACCCTGGCTGGAGCTGAGGTTTTGCTGCTGGCTGCCATGGCCTATGACCGATATGCTGCT | |
| GTTTGCAGACCTCTCCATTACCCACTGCTGATGAACCAGAGGGTGTGCCAGCTCCTGGTGT | |
| CAGCCTGCTGGGTTTTGGGAATGGTTGATGGTTTGTTGCTCACCCCCATTACCATGAGCTT | |
| CCCCTTTTGCCAGTCTAGGAAAATCCTGAGTTTTTTCTGTGAGACTCCTGCCCTGCTGAAGC | |
| TCTCCTGCTCTGACGTCTCCCTCTATAAGATGCTCACGTACCTGTGCTGCATCCTCATGCTT | |
| CTCACCCCCATCATGGTCATCTCCAGCTCATACACCCTCATCCTGCATCTCATCCACAGGAT | |
| GAA2TTCTGCCGCCGGCCGCAGGAAGGCCTTGGCCACCTGCTCCTCCCACATGATCATAGTG | |
| CTGCTGCTCTTCGGTGCTTCCTTCTACACCTACATGCTGCGGAGTTCCTACCACACAGCTGA | |
| GCAGGACATGATGGTGTCTGCCTTTTACACCATCTTCACTCCTGTGCTGAACCCCCTCATTT | |
| ACAGTCTCCGCAACAAAGATGTCACCAGGGCTCTGAGGAGCATGATGGAGTCAAGAATGA | |
| ACCAAGAAAAGTAG. | |
| AOLFR2S4 sequences: | |
| MTNTSSSDFTLLGLLVNSEAAGWFTVILAVFLGAVTANLVMIFLIQVDSRLHTPMTTLLSQLSI | (SEQ ID NO:477) |
| MDTLFICTTVPKLLADMVSKETISFVACGIQIFLTLTMIGSEFFLLGLMATDCTVAVCNPLRYP | |
| VLMNRKKCLLLAAGAWFGGSLDGFLLTPITMNVPTCGSRSTNIIFFCEIPAVLKLACADTSLYET | |
| LMYCCVLMLLIPISIISTSTSLILLTIHRMPSAEGRKKAFTTCSSHLTVVSIFTGAAFTTTVLPQS | |
| FHTPEQDKVVSAFTTIVTPMLNPLITSLRNKDVIGAIFKKVFACCSSAQKVATSDA. | |
| ATGACGAACACATCATCCTCTGACTTCACCCTCCTGGGGCTTGTGGTGAACAGTGAGGCTG | (SEQ ID NO:478) |
| CCGGGATTGTATTTACAGTGATCCTTGCTGTTTTCTTGGGGGCCGTGACTGCAAATTTGGT | |
| CATGATATTCTTGATTCAGGTGGACTCTCGCCTCCACACCCCCATGTACTTTCTGCTCAGTC | |
| AGCTGTCCATCATGGACACCCTTTTTCATCTGTACCACTGTCCCAAAACTCCTGGCAGACAT | |
| GGTTTTCTAAAGAGAAGATCATTTCCTTGTGGCCTGTGGCATCCAGATCTTCCTCTACCTG | |
| ACCATGATTGGTTCTGAGTTCTTCCTCCTGGGCCTCATGGCCTATGACTGCTACGTGGCTGT | |
| CTGTAACCCTCTGAGATACCCAGTCCTGATGAACCGCAAGAAGTGTCTTTTGCTGGCTGCT | |
| GGTGCCTGGTTTGGGGGCTCCCTCGATGGCTTTCTGCTCACTCCCATCACCATGAATGTCC | |
| CTTACTGTGGCTCCCGAAGTATCAACCATTTTTTCTGTGAGATCCCAGCAGTTCTGAAACT | |
| GGCCTGTGCAGACACGTCCTTGTATGAAACTCTGATGTACATCTGCTGTGTCCTCATGTTG | |
| GTCATCCGCATCTGTATCATCTCCACTTCCTACTCCCTCATCTTGTTAACCATCCACCGCAT | |
| GCCCTCTGCTGAAGGTCGCAAAAAGGCCTTCACCACTTGTTCCTCCCACTTGACTGTAGTT | |
| AGCATCTTCTATGGGGCTGCCTTCTACACATACGTGCTGCCCCAGTCCTTCCACACCCCCG | |
| AGCAGGACAAAGTAGTGTCAGCCTTCTATACCATTGTCACGCCCATGCTTAATCCTCTCAT | |
| CTACAGCCTCAGAAACAAGGACGTGATAGGGGCATTTAAAAAGGTATTTGCATGTTGCTCA | |
| TCTGCTCAGAAAGTAGCAACAAGTGATGCTTAG. | |
| AOLFR255 sequences: | |
| MEQSNTSVTADFILLGLFSNARFPWLLFALILLVFLTSIASNVVTILIIHIDSRLHTPMTTLLSQLS | (SEQ ID NO:479) |
| LRDILTSTIVPKMLVDQVMSQRAISFAGCTAQHFLTLTLAGAEFFLLGLMSTDRTVAICNPLH | |
| YPVLMSRTGWLIVAAAWLGGSIDGFLLTPVTMQFPFCASREINIHFFCEVPALLKLSCTDTSAY | |
| ETAMYVCCIMMLLIPFSVISGSTTRILITVTRMSEAEGRGKAVATCSSHMVVVSLFTGAAMYT | |
| YVLPIISTHTPEQDKAVSAFTTILTPMLNPLITSLRNKDVTGALQKVVGRCVSSGKVTTF. | |
| ATGGAGCAGAGCAATTATTCCGTGTATGCCGACTTTATCCTTTCTGGGTTTGTTCAGCAACG | (SEQ ID NO:480) |
| CCCGTTTGCCCTGGCTTCTCTTTGCCCTCATTCTCCTGGTCTTTTTGACCTCCATAGCCAGC | |
| AACGTGGTCAAGATCATTCTCATCCACATAGACTCCCGCCTCCACACCCCCATGTACTTCCT | |
| GCTCAGCCAGCTCTCCCTCAGGGACATCCTGTATATTTCCACCATTGTGCCCAAAATGCTG | |
| GTCGACCAGGTGATGAGCCAGAGAGCCATTTCCTTTGCTGGATGCACTGCCCAACACTTCC | |
| TCTACTTGACGTTAGCAGGGGCTGAGTTCTTGCTCCTAGGACTCATGTCCTATGATCGCTAC | |
| GTAGCGATGTGCAACCCTCTGCACTATCCTGTGGTGATGAGCCGCAAGATCTGCTGGTTGA | |
| TTGTGGCGGCAGGCTGGCTGGGAGGGTCTATCGATGGTTTCTTGCTCACCCCCGTCACCAT | |
| GCAGTTCCCCTTCTGTGCCTCTCGGGAGATCAACCACTTCTTCTGCGAGGTGCCTGCCCTTC | |
| TGAAGCTCTCCTGCACGGACACATCAGCCTACGAGACAGCCATGTATGTCTGGTGTATTAT | |
| GATGCTCCTCATCCCTTTCTCTGTCATCTCGGGCTCTTACACAAGAATTCTCATTACTGTTT | |
| ATAGGATGAGCGAGC3CAGAGGGGAGGGGAAAGGCTGTGQCCACCTGCTCCTCACACATGG | |
| TGGTTGTCAGCCTCTTCTATGGGGCTGCCATGTACACATACGTGCTGCCTCATTCTTACCAC | |
| ACCCCTGAGCAGGACAAAGCTGTATCTGCCTTCTACACCATCCTTACTCCCATGCTCAATC | |
| CACTCATTTACAGCCTTAGGAACAAGGATGTCACAGGGGCGCTACAGAAGGTTGTGGGGA | |
| GGTGTGTGTCCTCAGGAAAGGTAACCACTTTCTAA. | |
| AOLFR256 sequences: | |
| MGGKQPWVTEFILVGFQVGPALAILLCGLFSVFTTLTLLGNGVIFGIICLDSKLHTPMTTFLSHL | (SEQ ID NO:481) |
| AIIDMSTASNNVPKMLANLMNQKSTISFVPCIMQTFLTLAFAVTECLILVVMSTDRTVAICHPF | |
| QYTVIMSWRVCTILASTCWIISFLMALVHITHILRPPFCGPQTNHFICQIMSVFKLACAGPRLNQ | |
| VVLTAGSAFIVEGPLCLELVSNLHILSRHLEDPVMGRAADRLTLPAPSHLCMVGLLFGSTMVM | |
| YMAPKSRIHPEEQQKVLSLFTSLFNPMLNPLTTSLRNAEVKGALKRVLWKQRSK. | |
| ATGGGAGGCAAGCAGCCCTGGGTCACAGAATTCATCCTGGTGGGATTCCAGGTTGGTCCA | (SEQ ID NO:482) |
| GCACTGGCGATTCTCCTCTGTGGACTCTTCTGTGTCTTCTATACACTCACGCTGCTGGGGAA | |
| TGGGGTCATCTTTGGGATTATCTGCCTGGACTCTAAGCTTCACACACCCATGTACTTCTTCC | |
| TCTCAGACCTGGCCATCATTGACATGTCCTATGCTTCCAACAATGTTCCCAAGATGTTGGC | |
| AAACCTAATGAACCAGAAAAGCACCATCTCCTTTGITCCATGCATAATGCAGACTTTTTTG | |
| TATTTGGCTTTTGCTGTTACAGAGTGCCTGATTTTGGTGGTGATGTCCTATGATAGGTATGT | |
| GGCCATCTGCCACCCTTTCCAGTACACTGTGATCATGAGGTGGAGAGTGTGCACGATCCTG | |
| GCCTCAAGATGCTGGATAATTAGCTTTCTCATGGCTCTGGTCCATATAACTCATATTCTGAG | |
| GCCGCCTTTTTGTGGCCCACAAAAGATCAACCACTTTATCTGTCAAATCATGTCCGTATTCA | |
| AATTGGCCTGTGCTGGCCCTAGGCTCAACCAGGTGGTCCTATATGGGGGTTCTGCGTTCAT | |
| CGTAGAGGGGCCGCTCTGCCTGGAGCTGGTCTCCAACTTGCACATCCTGTCGCGCCATCTT | |
| GAGGATCCAGTAATGGGGAGGGCCGCAGACCGACTTACTCTTCCTGCTCCTTCCCACCTT | |
| GCATGGTGGGACTCGTTTTTGGCAGCACCATGGTCATGTACATGGCCCCCAAGTCCCGCCA | |
| CCCTGAGGAGCAGCAGAAGGTCCTTTCCCTGTTTTACAGCCTTTTCAACCCGATGCTGAAC | |
| CCCTTGATCTACAGCCTGAGGAACGCAGAGGTCAAGGGTGCCCTGAAAAGAGTGTTGTGG | |
| AAACAGAGATCAAAGTGA. | |
| AOLFR257 sequences: | |
| MESNQTWITEVILLGFQVDPALELFLFGFFLLFTSLTLMGNGHLGLITLDSRLHTPMTVFLSHL | (SEQ ID NO:483) |
| AIVDMSTASSTVPKMLANLVMHKKVISFAPCILQTFLTLAFATTECLILVMMCTDRTVAICHPL | |
| QYTLIMNWRVCTVLASTCWIFSFLLALVHITLILRLPFCGPQTNIIFFCQIMSVFKLACADTRLN | |
| QVVLFAGSAFILVGPLCLVLVSTLHILVAILRIQSGEGRRKAFSTCSSHLCVVGLFFGSAIVMYM | |
| APKSSHSQERRTLSLFTSLFNPILNPLITSLRNAEVKGALKRVLWKQRSM. | |
| ATGGAAAGCAATCAGACCTGGATCAGAGAAGTCATCCTGTTGGGATTCCAGGTGGACCCA | (SEQ ID NO:484) |
| GCTCTGGAGTTGTTCCTCTTTGGGTTTTTCTTGCTATTCTACAGCTTAACCCTGATGGGAAA | |
| TGGGATTATCCTGGGGCTCATCTACTTGGACTCTAGACTGCACACACCCATGTATGTCTTC | |
| CTGTCACACCTGGCCATTGTGGACATGTCCTATGCCTCGAGTACTGTCCCTAAGATGCTAG | |
| CAAATCTTGTGATGCACAAAAAAGTCATCTCCTTTGCTCCTTGCATACTTCAGACTTTTTTG | |
| TATTTGGCGTTTGCTATTACAGAGTGTCTGATTTTGGTGATGATGTGCTATGATCGGTATG | |
| TGGCAATCTGTCACCCCTTGCAATACACCCTCATTATGAAGTGGAGAGTGTGCACTGTCCT | |
| GGCCTCAACTTGCTGGATATTTAGCTTTCTCTTGGCTCTGGTCCATATTACTCTTATTCTGA | |
| GGCTGCCTTTTTGTGGCCCACAAAAGATCAACCACTTTTTCTGTCAAATGATGTCCGTATTC | |
| AAATTGGCCTGTGCTGACACTAGGCTCAACCAGGTGGTCCTATTTGCGGGTTCTGCGTTCA | |
| TCTTAGTGGGGCCGCTCTGCGTGGTGCTGGTGTCCTACTTGCACATCCTGGTGGCCATCTTG | |
| AGGATCCAGTCTGGGGAGGGCCGCAGAAAGGCCTTCTCTACCTGGTCCTCCCACCTCTGCG | |
| TGGTGGGGCTTTTCTTTGGCAGCGCCATTGTCATGTACATGGCCCCCAAGTCAAGCCATTC | |
| TCAAGAACGGAGGAAGATCCTTTCCCTGTTTTACAGCCTTTTCAACCCGATCCTGAACCCC | |
| CTCATCTACAGCCTTAGGAATGCAGAGGTGAAAGGGGCTCTAAAGAGAGTCCTTTGGAAA | |
| CAGAGATCAATGTGA. | |
| AOLFR259 sequences: | |
| MGDNQSRVTEFILVGFQLSVEMEVLLFWIFSLLTLFSLLANGMILGLICLDPRLRTPMTTFLSHL | (SEQ ID NO:485) |
| AVIDITTASSNLLNMLENLVKHKKTISFISCIMQMALTLTFAAAVCMILVVMSTDRFVAICHPL | |
| HYTVIMNWRVCTVLATTSWACGFSLALIINLILLLRLPFCGPQEVNHFFGEILSVLTACADTWIN | |
| EIFVFAGGVFVLVGPLSLMLISTMRILLMLTQSKEGRKKAFSTCSSHLCVVGLKTGMAIVIVVY | |
| LVPDNSQRQKQQTLTLFTSLFNPLLNPLITSLRNAQVKGALTRALQKKRTM. | |
| ATGGGGGACAACCAATCACGGGTCACAGAATTCATCGTGGTTGGATTCCAGCTCAGTGTG | (SEQ ID NO:486) |
| GAGATGGAAGTGCTCCTCTTCTGGATCTTCTCCCTGTTATATCTCTPCAGCCTGCTGGCAAA | |
| TGGCATGATCTTGGGGCTCATCTGTCTGGATCCCAGACTGCGCACCCCCATGTACTTCTTCC | |
| TGTCACACTTGGCCGTCATTGACATATACTATGCTTCCAGCAATTTGCTCAACATGCTGGA | |
| AAACCTAGTGAAACACAAAAAAACTATCTCGTTCATCTCTTGCATTATGCAGATGGCTTTG | |
| TATTTGACTTTTGCTGCTGCAGTGTGCATGATTTTGGTGGTGATGTCCTATGACAGATTTGT | |
| GGCGATCTGCCATCCCCTGCATTACACTGTCATCATGAACTGGAGAGTGTGCACAGTACTG | |
| GCTATTACTTCCTGGGCATGTGGATTTTCCCTGGCCCTCATAAATCTAATTCTCCTTCTAAG | |
| GCTGCCCTTCTGTGGGCCCCAGGAGGTGAACCACTTCTTCGGTGAAATTCTGTCTGTCCTC | |
| AAACTGGGCTGTGCAGACACCTGGATTAATGAAATTTTTGTCTTTGCTGGTGGTGTGTTTG | |
| TCTTAGTCGGGCCCCTTTCCTTGATGCTGATCTCCTACATGCGCATCCTCTTGGCCATCCTG | |
| AAGATCCAGTCAAAGGAGGGGCGCAAAAAAGCCTTTTCCACGTGCTCCTCCCACCTCTGTG | |
| TGGTTGGGCTTTACTTTGGCATGGCCATGGTGGTTTACCTGGTCCCAGACAACAGTCAACG | |
| ACAGAAGCAGCAGAAAATTCTCACCCTGTTTTACAGCCTTTTCAACCCATTGCTGAACCCC | |
| CTCATCTACAGCCTGCGGAATGCTCAAGTGAAGGGTGCCTTATACAGAGCACTGCAGAAA | |
| AAGAGGACCATGTGA. | |
| AOLFR24B sequences: | |
| MPSLNDTHFTPPFFLLLGIPGLDTLHIWISFPFCIVTLIAIVGNMTILFVIKTEHSLHQPMFTTLAM | (SEQ ID NO:487) |
| LSMIDLGLSTSTIPKMLGIFWFNLQEISFGGCLLQMFFIHMFTGMETVLLVVMATDRFVAICNP | |
| LQYTMILTNKTISILASVVVGRNLVLVTPFVFLILRLPFCGHNIVPHTTCEHRGLAGLACAPIKIN | |
| IIYGLMVISTIVDVILLASSTVLILRAVFRLPSQDKTRLKAFNTCGSHVCVMLCFTTPAFFSFMTH | |
| RFGQNIPHTIHILLANLTVVVPPALNPVITGVRTKQIREQIVTFVQKE | |
| ATGCCTTCTATCAATGACACCCACTTCTATCCCCCCTTCTTCCTCCTGCTAGGAATACCAGG | (SEQ ID NO:488) |
| ACTGGACACTTTACATATCTGGATTTCTTTCCCATTCTGTATTGTGTACCTGATTGCCATTG | |
| TGGGGAATATGACCATTCTCTTTGTGATCAAAACTGAACATAGTCTACACCAGCCCATGTT | |
| CTACTTCCTGGCCATGTTGTCTATGATTGATCTGGGTCTGTCCACATCCACTATCCCCAAAA | |
| TGCTAGGAATCTTGTGGTTCAACCTCCAAGAGATCAGCTTTGGGGGATGCCTTCTTCAGAT | |
| GTTCTTTATTCACATGTTTACAGGCATGGAGACTGTTGTGTTGGTGGTCATGGCTTATGACC | |
| GCTTTGTTGCCATCTGCAACCCTCTCCAGTACACCATGATCCTCACCAATAAAACCATCAG | |
| TATCCTAGCTTCTGTGGTTTGTTGGAAGAAATTTAGTTGTTGTAACCCCATTTGTGTTTCTCA | |
| TTCTGCGTCTGCCATTCTGTGGGCATAACATCGTACGTCACACATACTGTGAGCACAGGGG | |
| TCTGGCCGGGTTGGCCTGTGCACCCATTAAGATCAACATAATCTATGGGCTCATGGTGATT | |
| TCTTATATTATTGTGGATGTGATCTTAATTGCCTGTTCCTATGTGCTTATCCTTAGAGCTGT | |
| TTTTCGCCTTCCCTCTCAAGATGTCCGACTAAAGGCCTTCAATACCTGTGGTTCTCATGTCT | |
| GTGTTATGCTGTGCTTTTAGACACCAGCATTTTTTTGTTTTATGACACATCGTTTTGGCCAA | |
| AACATTCCCCACTATATCCATATTCTTTTTGGCTAACCTGTATGTGGTTGTGCCACCTGCCCT | |
| TAACCCTGTCATTTATGGAGTCAGGACCAAGCAGATGCGAGAGCAAATTGTGAAAATATTT | |
| GTACAGAAAGAATAA | |
| AOLFR33B sequences: | |
| MLHTNNTQFIIPSTFLVVGVPGLEDVHVWIGFPFFAVTLTALLGNIIILFVIQTEQSLIHQPMFYFL | (SEQ ID NO:489) |
| AMLAGTDLGLSTATIPKMLGIFWFNLGEIAFGACITQMTTIHICTGLESVVLTVTGLDRTAICNP | |
| LRYSMILTNKVIAILGIVIIVRTLVFVTPFTFLTLRLPFCGVRIIPHTTCEHMGLAKLACASINVIY | |
| GLIAFSVGTDISVIGFSTVQILRAVFHLPAWDARLKALSTCGSHVCVMIAFTLPALFSFMTHRF | |
| GHNIPHTIHILLANLTVVFPPALNSVITGVKTKQIREQVLRILNPKSFWHFDPKRIFHNNSVRQ | |
| ATGCTTCATACCAACAATACACAGTTTCACCCTTCCACCTTCCTCGTAGTGGGGGTCCCAG | (SEQ ID NO:490) |
| GGCTGGAAGATGTGCATGTATGGATTGGCTTCCCCTTCTTTGCGGTGTATCTAACAGGCCT | |
| TCTAGGGAACATCATTATCCTGTTTGTGATACAGACTGAACAGAGCCTGCACCAACCCATG | |
| TTTTACTTCCTAGCCATGTTGGCCGGCACTGATCTGGGCTTGTCTACAGCAACCATCCCCA | |
| AGATGCTGGGAATTTTCTGGTTTAATCTTGGAGAGATTGCATTTGGTGCCTGCATCACACA | |
| GATGTATACCATTCATATATGCACTGGCCTGGAGTCTGTGGTACTGACAGTCACGGGCATA | |
| GATCGCTATATTGCCATCTGCAACCCCCTGAGATATAGCATGATCCTTACCAACAAGGTAA | |
| TAGCCATTCTGGGCATAGTCATCATTGTCAGGACTTTTGGTATTTGTGACTCCATTCACATTT | |
| CTCACCCTGAGATTGCCTTTCTGTGGTGTCCGGATTATCCCTCATACCTATTGTGAACACAT | |
| GGGCTTGGCAAAGTTAGCTTGTGCCAGTATTAATGTTATATATGGATTGATTGCCTTCTCA | |
| GTGGGATACATGACATTTCTGTGATTGGATTTTCCTATGTCCAGATCGTCCGAGCTGTCTT | |
| CCATCTCCCAGCCTGGGATGCCCGGCTTAAGGCACTCAGGACATGTGGCTCTCACGTCTGT | |
| GTTATGTTGGCTTTCTACCTGCGAGCCCTCTTTTCCTTCATGACACACCGCTTTGGCCACAA | |
| CATCCCTCATTACATCCACATTCTTCTGGCCAATCTGTATGTGGTTTTTCCCCCTGCTCTTA | |
| ACTCTGTTATGTATGGGGTCAAAACAAAACAGATACGAGAGCAGGTACTTAGGATACTCA | |
| ACCCTAAAAGCTTTTGGCATTTTGACCCCAAGAGGATCTTCCACAACAATTCAGTTAGACA | |
| ATAA | |
| AOLFR112B sequences: | |
| MKNKTVLTEFILLGLTDVPELQVAVFTFLFLATLLSILGNLTILILTLLDSHLQTPMTTFLRMFSF | (SEQ ID NO:491) |
| LEISFTNIFWRVLISITTGNKSISFAGCFTQTTFAMFLGATEFTLLAAMSTDRTVAICKPLHYTTI | |
| MSSRICIQLIFCSWLGGLMAIIPTTLMSQQDFCASNRLNHTTCDTEPLLELSCSDTSLIEKVVFL | |
| VASVTLVVTLVLVILSTAFIIKTILKLPSAQQRTKMSTCSSHMIVISLSTGSCMFMTTTNPSAKEG | |
| DTFNKGVALLITSVAPLLNPFITTLRNQQVKQPFKDMVKKLLNL | |
| ATGAAAAATAAAACCGTGTTAACTGAGTTTATCCTTCTGGGTCTAACAGATGTCCCTGAAC | (SEQ ID NO:492) |
| TCCAGGTGGCAGTTTTCACCTTTCTTTTGCTTGCGTATTTACTCAGCATCCTTGGAAATCTG | |
| ACTATCCTCATCCTCACCTTGCTGGACTCCCACCTTTCAGACTCCCATGTATTTCTTTCTCCG | |
| GAACTTCTCCTTCTTGGAAATTTCCTTCACAAACATCTTCATTCCAAGGGTCCTGATTAGCA | |
| TCACAACAGGGAACAAGAGTATCAGCTTTGCTGGCTGCTTCACTCAGTATTTCTTTGCCAT | |
| GTTCCTTGGGGCTACAGAGTTTTACCTTCTGGCTGCCATGTCCTATGACCGCTATGTGGCC | |
| ATCTGCAAACCTCTGCATTACACCACCATCATGAGCAGCAGAATCTGCATCCAGCTGATTT | |
| TCTGCTCTTGGCTGGGTGGGCTAATGGCTATTATACCAACAATCACCCTGATGAGTCAGCA | |
| GGACTTTTGTGCATCCAACAGACTGAATCATTACTTCTGTGACTATGAGCCTCTTCTGGAA | |
| CTCTCATGTTCAGACACAAGCCTCATAGAGAAGGTTGTCTTTCTTGTGGCATCTGTGACCC | |
| TGGTGGTCACTCTGGTGCTAGTGATTCTCTCCTATGCATTCATTATCAAGACTATTCTGAAG | |
| CTCCCCTCTGCCCAACAAAGGACAAAAGCCTTTTCCACATGTTCTTCCCACATGATTGTCAT | |
| CTCCCTCTCTTACGGAAGCTGCATGTTTATGTACATTAATCCCTCTGCAAAAGAAGGGGAT | |
| ACATTCAACAAGGGAGTAGCTCTACTCATTACTTCAGTTGCTCCTTTGTTGAACCCCTTTAT | |
| TTACACCCTAAGGAACCAACAGGTAAAACAACCCTTCAAGGATATGGTCAAAAAGCTTCT | |
| GAATCTTTAA | |
| AOLFRL30B sequences: | |
| MEGKNQTAPSEFIILGFDHLNELQTLLFTIFFLTTCTLGGNVFIIVVTIADSHLHTPMTTTLGNL | (SEQ ID NO:493) |
| ALIDICTTTTNVPQMMVHLLSEKTISTGGCVTQLFAFIFFVGSECLLLAAMATDRTAICKPLR | |
| YSHMNKALCSWLAASCWTCGFLNSVLHTVLTFIILPFCGNNQINTTFCDIPPLLILSCGDTSLNE | |
| LALLSIGILISWTPFLCIILSTLTISTILPIRSSEGRHKAFSTCASHLLIVILTTGSAIFTTVRPISSYS | |
| LEKDRLISVLTSVVTPMLNPVITTLRNKDIKEAVKAIGRKWQPPVFSSDI | |
| ATGGAAGGAAAGAATCAAACAGCTCCATCTGAATTCATCATCTTGGGGTTCGACCACGTGA | (SEQ ID NO:494) |
| ATGAATTGCAGTATTTACTCTTCACCATCTTCTTTCTGACCTACATATGCACTTTAGGAGGC | |
| AATGTTTTTATCATTGTGGTGACCATAGCTGATTCCCACCTACACACACCCATGTATTATTT | |
| CCTAGGAAATCTTGCCCTTATTGACATCTGCTACACTACTACTAATGTCGCCCAGATGATG | |
| GTGCATCTTCTGTCAGAGAAGAAAATCATTTCCTATGGAGGCTGTGTGACCCAGCTCTTTG | |
| CATTCATTTTCTTTGTTGGCTCAGAGTGTCTCCTCCTGGCAGCAATGGCATATGATCGATAT | |
| ATTGCTATCTGTAAGCCGTTAAGGTACTCATTTATTATGAACAAGGCCCTGTGCAGCTGGT | |
| TAGCAGCCTCATGCTGGACATGTGGGTTTCTCAACTCAGTGTTGCACACCGTTCTGACCTT | |
| CCACCTGCCCTTCTGTGGTAACAATCAGATCAATTATTTCTTCTGTGACATACCTCCCTTGC | |
| TCATCTTGTCTTGTGGTGATACTTCCCTCAATGAACTGGCTTPGCTGTCCATTGGGATCCTC | |
| ATAAGCTGGACTCCTTTCCTGTGCATCATCCTTTCCTACCTTTACATCATGTCCACCATCCT | |
| GAGGATCCGTTCCTCTGAGGGGAGGCACAAAGCCTTTTCCACCTGTGCCTCCCACCTGCTC | |
| ATTGTTATTCTCTATTATGGCAGTGCTATCTTCACGTATGTGAGGCCCATGTGATCTTACTC | |
| TCTAGAGAAAGATAGATTGATCTCAGTGCTGTATAGTGTTGTCACACCCATGCTGAATCCT | |
| GTAATTTATACGCTAAGGAATAAGGACATCAAAGAGGCTGTGAAGGCCATAGGGAGAAAG | |
| TGGCAGCCACCAGTTTTCTCTTCTGATATATAA | |
| AOLFR142B sequences: | |
| MARKDMAHINCTQATEFILVGLTDHQELKMPLFVLFLSITLFTVVGNLGLILLIRADTSLNTPM | (SEQ ID NO:495) |
| YPFLSNLAEVDFCTSSVITPKMLGNFLTKQNVISFDACATQLGCFLTFMISESLLLASMATDRY | |
| VAICNPLLTMVVMTPGICIQLVAVPTSTSFLMAIFHTILTFRLSTCHSNWNHFTCDDMPLLRL | |
| TCSDTRFKQLWIFACAGIMFISSLLIVFVSTMFIISAILRMHSAEGRQKAFSTCGSHMLAVTIFYG | |
| TLIFMTLQPSSSHALDTDKMASVFTTVIIPMLNPLITSLQNKEVKEALKKIIINKN | |
| ATGGCCAGAAAAGATATGGCTCACATCAATTGCACCCAGGCGACAGAGTTTATTCTTGTGG | |
| GCCTCACAGACCATCAGGAGTTGAAGATGCCCCTCTTTGTGCTATTCTTATCCATCTACCTC | |
| TTCACAGTGGTAGGCAACTTGGGTTTGATCCTACTCATTAGAGCGGATACAAGTCTCAACA | |
| CACCAATGTACTTCTTTCTTAGCAACCTAGCTTTTGTGGATTTCTGTTACTCTTCTGTCATT | |
| ACACCCAAAATGCTTGGGAATTTCTTGTACAAACAAAATGTTATATCCTTTGATGCATGTG | |
| CTACTCAACTGGGCTGCTTTCTCACCTTCATGATATCAGAATCCTTGCTACTGGCTTCCATG | |
| GCCTATGACCGATATGTGGCCATTTGTAACCCTCTATTGTATATGGTTGTAATGACTCCAG | (SEQ ID NO:496) |
| GAATCTGCATTCAACTTGTAGCAGTTCCTTATAGCTATAGCTTCCTAATGGCACTATTTCAC | |
| ACCATCCTCACCTTGCGCCTCTCCTATTGCCACTCCAACATTGTCAACCATTTCTATTGTGA | |
| TGACATGCCTCTCCTCAGGCTAACTTGCTCAGACACTCGCTTCAAACAGCTCTGGATCTTT | |
| GCCTGTGCTGGTATCATGTTCATTTCCTCCCTTCTGATTTGTCTTTGTCTCCTACATGThCATC | |
| ATTTCTGGCATGCTGAGGATGCATTCAGCTGAGGGAAGACAGAAGGCTTTCTCGACGTGTG | |
| GCTCTCACATGCTGGCAGTCACCATATTCTATGGGACCCTCATTTTTATGTACTTACAGCCT | |
| AGCTCTAGCCATGCCCTGGACACAGACAAGATGGCCTCTGTCTTCTACACAGTGATCATTC | |
| CCATGTTGAATCCCTTAATCTATAGCCTCCAGAATAAGGAGGTGAAAGAAGCTCTGAAGA | |
| AAATCATTATCAATAAAAACTAG | |
| AOLFR171C sequences: | |
| MAEVNIITVTVFILKGITNRPELQAPCFGVFLVITLVTVLGNLGLITLITDTRLHTPMTTTLSHL | (SEQ ID NO:497) |
| AFVDLCTSSATTPKMMVNFVVERNTIPFHACATQLGCFLTFMITECFLLASMATDCTVAICSPL | |
| HTSTLMSRRVCIQLVAVPTTSFLVALFHTVITFRLTTCGPNLINHFTCDDLPFLALSCSDTHMK | |
| EILIFAFAGFDMISSSSIVLTSTFIIAAILRIRSTQGQIIKAISTCGSIIMVTVTIFTGTLIFMTLQPKS | |
| NHSLDTDKMASVFTTVVIPMLNPLITSLRT\TKEVKDASKKALDKGCENLQILTFLTRKLT | |
| ATGGCTGAAQTTAATATCATTTATGTCACTGTATTCATTCTGAAAGGAATTACCAACCGGC | (SEQ ID NO:498) |
| CAGAGCTTCAGGCCCCGTGCTTTGGGGTGTTTTTAGTTATCTATCTGGTCACAGTGCTGGG | |
| GAATCTTGGGTTGATTACTTTAATCAAGATTGATACTCGACTCCACACACCTATGTACTATT | |
| TCCTCAGGCACCTGGCCTTTGTTGACCTTTGTTACTCCTCTGCTATTACACCGAAGATGATG | |
| GTGAATTTTGTTGTGGAACGCAACACCATTCCTTTCCATGCTTGTGCAACCCAACTGGGTT | |
| GTTTTTCTCACCTTCATGATCACTGAGTGTTTCCTTCTAGCCTCCATGGCCTACGATTGCTAT | |
| GTCGCCATCTGTAGTCCCCTGCATTATTCAACACTGATGTCAAGAAGAGTCTGCATTCAAC | |
| TGGTGGCAGTTCCATATATATACAGCTTCCTGGTTGCCCTGTTCGACACCGTTATCACTTTC | |
| CGTCTGACTTACTGTGGCCCAAACTTAATTAAGCATTTCTATTGTGATGACCTCCCCTTCTT | |
| AGCTCTGTCCTGCTCAGACACACACATGAAGGAAATTCTGATATTTGCCTTTGCTGGCTTT | |
| GATATGATCTCTTCCTCTTCCATTGTCCTCACCTCCTACATCTTTATTTATTGCCGCTATCCTA | |
| AGGATCCGCTCTACTCAGGGGCAACACAAAGCCATTTGCACCTGTGGCTCGCATATGGTGA | |
| CTGTCACTATTTTCTATGGCACACTGATCTTTATGTACCTACAGCCCAAATCAAATCACTCC | |
| TTGGACACAGACAAGATGGCTTCTGTATTTTACACAGTGGTGATCCCCATGTTAAACCCCC | |
| TAATCTATAGTCTAAGGAACAAAGAAGTGAAAGATGCCTCAAAGAAAGCCTTGGATAAAG | |
| GTTGTGAAAACTTACAGATATTAACATTTTTAAAAATAAGAAAACTTTATTAA | |
| AOLFR225B sequences: | |
| MKNRTMFGEFILLGLTNQPELQVMIFIFLFLTTMLSILGNLTIITLTLLDPHLQTPMTTFLRNFSF | (SEQ ID NO:499) |
| LEISFTSIFIPRFLTSMTTGNKVISFAGCLTQTTFATFLGATEFTLLASMSTDRTVAICKPLHTLTI | |
| MSSRVCIQLVECSWLGGFLAILPPIILMTQVDFCVSNILMHTTCDTGPLVELACSDTSLLELMVI | |
| LLAVVTLMVTLVLVTLSTTTIRTILRIPSAQQRTKAFSTCSSHMIVISLSTGSCMIFMTThPSAKE | |
| GGAFNKGIAVLITSVTPLLNPFITTLRNQQVKQAFKDSVKKWKL | |
| ATGAAAAACAGAACCATGTTTGGTGAGTTTATTCTACTGGGCCTTACAAATCAACCTGAAC | (SEQ ID NO:500) |
| TCCAAGTGATGATATTCATCTTTCTGTTCCTCACCTACATGCTAAGTATCCTAGGAAATCTG | |
| ACTATTATCACCCTCACCTTACTAGACCCCCACCTCCAGACCCCCATGTATTTCTTCCTCCG | |
| GAATTTCTCCTTCTTAGAAATTTCCTTCACATCCATTTTTATTCCCAGATTTCTGACCAGCA | |
| TGACAACAGGAAATAAAGTTATCAGCTTTGCTGGCTGCTTGACTCAGTATTTTTTTGCTAT | |
| ATTTCTTGGAGCTACCGAGTTTTACCTCCTGGCCTCCATGTCTTATGATCGTTATGTGGCCA | |
| TCTGCAAACCCTTGCATTACCTGACTATTATGAGCAGCAGAGTCTGCATACAACTAGTGTT | |
| CTGCTCCTGGTTGGGGGGATTCGTAGCAATCTTAGCACCAATCATCCTGATGACCCAGGTA | |
| GATTTCTGTGTCTCCAAGATTCTGAATCACTATTACTGTGACTATGGGCCTCTCGTGGAGCT | |
| TGCCTGCTCAGACACAAGCCTCTTIAGAACTGATGGTCATCCTCTTGGCCGTTGTGACTCTC | |
| ATGGTTACTCTGGTGCTGGTGACACTTTCTTACACATACATTATCAGGACTATTCTGAGGA | |
| TCCCTTCTGCCCAGCAAAGGACAAAGGCCTTTTCCACTTGTTCCTCCCACATGATTGTCATC | |
| TCCCTCTCTTATGGCAGCTGCATGTTATGTACATfAATCCTTCTGCAAAAGAAGGAGGTG | |
| CTTTCAACAAAGGAATAGCTGTACTCATTACTTCGGTTACTGCCTTACTGAATCCCTTCATA | |
| TATACTTTAAGAAATCAGGAAGTGAAACAAGCTTTCAAGGACTCAGTCAAAAAGATTGTG | |
| AAACTTTAA | |
| AOLFR274B sequences: | |
| MEFVFLATPSCPELHILSFLGVSLVTGLIITGNILIVVSIIITETCLCTSMTTTLGSLSGIEICTTAV | (SEQ ID NO:501) |
| VVPHILANTLQSEKTTLLGCATQMAFFIALGSADCFLLAAIVIATDRTVAICHPLQTPLLMTLTL | |
| CVHLVVASVISGLFLSLQLVAFIFSLPFCQAQGIEHFFCDVPPVMHVVCAQSHIHEQSVLVAAIL | |
| AIAVPFFLITTSTTFIVAALLTHSAAGRHRAFSTCSSHLTVVLLQTGCCAFMTLCPSSSTNPKQ | |
| DRFISLVTTLGTPLLNPLITALRNSEMKGAVGRVLTRNCLSQNS | |
| ATGGAATTTGTGTTCCTGGCCTATCCCTGCTGCCCAGAACTGCATATTCTGTCCTTCCTTGG | (SEQ ID NO:502) |
| GGTCAGCCTGGTTTTATGGTTTGATCATCACTGGGAACATTCTCATTGTGGTGTCCATTCAC | |
| ACAGAAACCTGTCTATGCACATCCATGTACTATTTCCTGGGCAGCCTTTCTGGGATTGAAA | |
| TATGCTACACTGCAGTGGTGGTGCCCCATATCCTGGCCAACACCCTACAGTCAGAGAAGAC | |
| CATCACTCTCCTGGGCTGTGCCACCCAGATGGCTTTCTTCATTGCACTGGGCAGTGCTGAT | |
| TGCTTCCTCTfGGCTGCCATGGCCTATGACCGCTATGTGGCCATTTGCCACCCGTiTGCAGTA | |
| CCCTCTCCTCATGACATTGACTCIITGTGTCCACTTGGTTGTGGCATCAGTCATCAGTGGTC | |
| TGTTCCTGTCCTTACAACTGGTGGCCTTCATCTTCTCTCTGCCATTCTGCCAGGCTCAGGGC | |
| ATTGAGCACTTCTTTTGTGATGTGCCACCAGTCATGCATGTTGTTTGTGCTCAGAGTCACAT | |
| TCATGAGCAGTCAGTGCTGGTGGCAGCCATACTAGCCATTGCTGTGCCTTTCTTCCTCATC | |
| ACCACCTCCTACACCTTCATAGTGGCTGCTCTGCTCAAGATCCACTGGGCTGCTGGCCGCC | |
| ACCGGGCGTTCTCCACCTGCTCTTCCCACCTCACTGTGGTGCTGCTGCAGTATGGCTGCTGT | |
| GCCTTCATGTACCTGTGCCCCAGCTCCAGCTACAACCCCAAGCAAGATCGGTTCATCTCAC | |
| TGGTGTACACATTGGGAACCCCACTGCTCAACCCACTTATCTATGCCCTGAGGAACAGTGA | |
| GATGAAAGGGGCCGTAGGGAGAGTTCTTACCAGGAACTGCCTTETCGCAGAAGAGCTAG | |
| AOLFR276B sequences: | |
| MGGFGTNISSTTSFTLTGFPEMKGLEHWLAALLLLLTAISFLGNILILFIIKEEQSLHQPMTTTLS | (SEQ ID NO:503) |
| LFSVNDLGVSFSTLPTVLAAVCFHAPETTFDACLAQMFFIHFSSWTEFGILLAMSFDHTVAICNP | |
| LRTATVLTDVRVAHNGISIVIRSFCMVFPLPFLLKRLPFCKASVVLAHSTCLHADLmLPWGDT | |
| TINSMTGLFIVISAFGVDSLLILLSTVLILHSVLAIASRGERLKTLNTCVSHITAVLIFTVPMVSVS | |
| MVLIRFGRIIAPETVHKFMSLCTSNALPNTLFHQD | |
| ATGGGGGGCTTTGGGACTAACATCTCAAGTACTACCAGCTTCACTCTAACAGGCTTCCCTG | (SEQ ID NO:504) |
| AGATGAAGGGTCTGGAGCACTGGCTGGCTGCCCTTCTGCTGCTGCTTTATGCTATTTCCTT | |
| CCTGGGCAACATCCTCATCCTCTTTATCATAAAGGAAGAGCAGAGCTTGCACCAGCCAATG | |
| TACTACTTTCCTGTCTCTTTTTTCTGTTAATGACCTGGGTGTGTCCTTTTCTACATTGCCCACT | |
| GTACTGGCTGCTGTGTGTTTTCATGCCCCAGAGACAACTTTTGATGCCTGCCTGGCCCAGA | |
| TGTTCTTCATCCACTTTTCCTCCTGGACAGAGTTTGGCATCCTACTGGCCATGAGTTnGAC | |
| CACTATGTGGCCATCTGTAACCCGCTGCGCTATGCCACAGTGCTCACTGATGTCCGTGTGG | |
| CCCACAATGGCATATCCATTGTCATCCGCAGCTTCTGCATGGTATTCGCACTTCCCTTCCTC | |
| CTGAAGAGACTGCCTTTCTGTAAGGCCAGTGTGGTACTGGCCCATTCCTACTGTCTGCATG | |
| CAGACCTGATTCGGCTGCCCTGGGGAGACACTACCATCAACAGCATGTATGGCCTGTTCAT | |
| TGTCATGTCTGCCTTTGGTGTAGATTCAGTGCTCATCCTCCTCTCGTATGTGCTCATTCTAC | |
| ATTCTGTGCTGGCCATTGCCTCCAGGGGTGAGAGGCTTAAGACACTCAACACATGTGTGTC | |
| ACATATCTATGCAGTGCTGATCTTCTATGTGCCTATGGTTAGTGTGTCCATGGTTCATCGAT | |
| TTGGGAGGCATGCTCGTGAATATGTGCACAAGTTCATGTCTCTTTGTACCTCCAATGCTCT | |
| ACCCAATTATCTATTCCATCAAGACTAA | |
| AOLFR31TB sequences: | |
| MDWENCSSLTDFFLLGITNNPEMKVTLFAVPLAVTTNFSANLGMIVLIRiMDTQLHTPMTEFLS | (SEQ ID NO:505) |
| HLSFCDLCTSTATGPKMLVDLLAKNKSIPFTGCAIQFLVFCIFADSECLLLSVMAFDRTKAIINP | |
| LLTTVNMSSRVCTLLLTGVTLVGIADALIHMTLAFRLCFCGSNEINHFFCDIPPLLLLSRSDTQV | |
| NELVLFTVFGFIELSTISGVFISTCTILSVLEIHSAEGRFKALSTCTSHLSAVMFQGTLLFMTTRP | |
| SSSTSLDQDKMTSLFTTLVVPMLNPLITSLRNKDVKEALKKLKNTLF | |
| ATGGACTGGGAAAATTGCTCCTCATTAACTGATTTTTTTGTCTTGGGAATTACCAATAACCC | (SEQ ID NO:506) |
| AGAGATGAAAGTGACCCTATTTGCTGTATTCTTGGCTGTTTATATCATTAATTTCTCAGCAA | |
| ATCTTGGAATGATAGTTTTTAATCAGAATGGATTACCAACTTCACACACCAATGTATTTCTT | |
| CCTCAGTCATCTGTCTTTfTCTGTGATCTCTGCTATTCTACTGCAACTGGGCCCAAGATGCTGG | |
| TAGATCTACTTGCCAAGAACAAGTCAATACCCTTCTATGGCTGTGCTCTGGAATTCTTGGT | |
| CTTCTGTATCTTTGCAGATTGTGAGTGTCTACTGCTGTCAGTGATGGGCTTTGATCGGTACA | |
| AGGGCATCATCAACCCCCTGCTCTATACAGTCAACATGTCTAGCAGAGTGTGCTATCTACT | |
| CTTGACTGGGGTTTATCTGGTGGGAATAGCAGATGCTTTGATACATATGACACTGGCCTTC | |
| CGCCTATGCTTCTGTGGGTCTAATGAGATTAATCATTTTCTTCTGTGATATCCCTCCTCTCTT | |
| ATTACTCTCTCGCTCAGATACACAGGTCAATGAGTTAGTGTTATTCACCGTCTTTGGTTTTA | |
| TTGAACTGAGTACCATTCAGGAGTTTTCATTTCTTATTGTTATATCATCCTATCAGTCTTG | |
| GAGATACACTCTGCTGAGGGGAGGTTCAAAGCTCTCTCTACATGCACTTCCCACTTATCTG | |
| CGGTTGCAATTTTCCAGGGAACTCTGCTCTTTATGTATETTCCGGCCAAGTTCTTCCTACT | |
| CTAGATCAAGATAAAATGACCTCATPTGTTTTACACCCTTGTGGTTCCCATGTTGAACCCCCT | |
| GATTTATAGCCTGAGGAACAAGGATGTGAAAGAGGCCCTGAAAAAACTGAAAAATAAAAT | |
| TTTATTTTAA | |
| AOLFR314 sequences: | |
| MEVKNCCMVTEFILLGIPHTEGLEMTLFVLFLPFTACTLLGNVSILVAVMSSARLHTPMTTFLG | (SEQ ID NO:507) |
| NLSVFDMGFSSVTCPKMLLTLMGLSRLISTKDGVCQLFFFHFLGSIECFLFTVMATDRFTAICT | |
| PLRTTVIMNPRTGVALAVGTWLLGCIHSSILTSLTFTLPTCGPNEVDHFFCDIPALLPLACADTSL | |
| AQRVSFTNVGLISLVCFLLILLSTTRiITISILSIRTTEGRRRAFSTCSALHLIAILCATGPIITVTLQPT | |
| PNPMLGTVVQILMNLVGPMLNPLTTTLRNKEVKTALKTILHRTGHVPES | |
| ATGGAGGTGAAGAACTGCTGCATGGTGACAGAGTTCATCCTTTTGGGAATCCCACACACA | (SEQ ID NO:508) |
| GAGGGGCTGGAGATGACACTTTTTGTCTTATTCTfGGCCTTCTATGCCTGCACTCTACTGGG | |
| AAATGTGTCTATCCTTGTTGCTGITATGTCTTCTGCTCGGCTTCACACACCTATGTATTTCT | |
| TCCTGGGAAACTTGTCTGTGTTTGACATGGGTTITCTCCTCAGTGACTTGTCCCAAAATGCT | |
| GCTCTACCTTATGGGGCTGAGCCGACTCATCTCCTACAAAGACTGTGTCTGCCAGCTTTTCT | |
| TCTTCCATTTCCTCGGGAGCATTGAGTGCTTCTTGTTTACGGTGATGGCCTATGACCGCTTC | |
| ACTGCCATCTGTTATCCTCTGCGATACACAGTCATCATGAACCCAAGGATCTGTGTGGCCC | |
| TGGCTGTGGGCACATGGCTGTTAGGGTGCATTCATTCCAGTATCTTGACCTCCCTCACCTTC | |
| ACCTTGCCATACTGTGGTCCCAATGAAGTGGATCACTTCTTGTGTGACATTCCAGCACTGTT | |
| GCCCTTGGCCTGTGCTGACACATCCTTAGCCGAGAGGGTGAGCTTCACCAACGTTGGCCTC | |
| ATATCTCTTGTCTGCTTTCTGCTAATTCTTTTATCCTACACTAGAATCACAATATCTATCTT | |
| AAGCATTCGTACAACTGAGGGCCGTCGCCGTGCCTTTCTCCACCTGCAGTGCTCACCTCATT | |
| GCCATCCTCTGTGCCTATGGGCCCATCATCACTGTCTACCTGCAGCCCACACGCAACCCCA | |
| TGCTGGGAACCGTGGTACAAATTCTGATGAATCTGGTAGGACCAATGCTGAACCCTTTGAT | |
| CTATACCTTGAGGAATAAGGAAGTAAAAACAGCCCTGAAAACAATATTGCACAGGACAGG | |
| CCATGTTCCTGAGAGTTAG | |
| AOLFR324B sequences: | |
| MPIAI\TDTQFHTSSFLLLGIPGLEDVHIWIGFPFFSVTLIALLGNAAIFFVIQTEQSLHEPMTTCLA | (SEQ ID NO:509) |
| MLDSIDLSLSTATIPKMLGIFWFNIKEISFGGTLSQMFFIIIFFTVMESIVLVAMAFDRTAICKLPL | |
| WTTMILTSTISLIAGIAVLRSLTMVIIPLVFLLLRLPFCGHRIIPHTTCEHMGIARLACASIKVNIM | |
| FGLGSISLLLLDVLLIILSHWILTAVFCLPSWEARLKALNTCGSHIGVILAFSTPAFFSFFTHCFGH | |
| DIP QTHIFLANLTVVVPPTLNIPVITGVRTTHIRETVLRIFFKTDH | |
| ATGCCTATAGCTAACGACACCCAGTTCCATACTTCTTCATTCCTACTGCTGGGTATCCCAGG | (SEQ ID NO:510) |
| GCTAGAAGATGTGCACATCTGGATTGGATTCCCTTTTTTCTCTGTGTATCTTATTGCACTCC | |
| TGGGAAATGCTGCTATCTTCTTTGTGATCCAAACTGAGCAGAGTCTCCATGAGCCCATGTA | |
| CTACTGCCTGGCCATGTTGGATTCCATTGACCTGAGCTTGTGTACGGCCACCATTGCCAAA | |
| ATGCTGGGCATCTTCTGGTTCAATATCAAGGAAATATCTTTTGGAGGCTACCTTTCTCAGA | |
| TGTTCTTCATCCATTTCTTCACTGTCATGGAGAGCATCGTATTGGTGGCCATGGCCTTTGAC | |
| CGCTACATTGCCATTTGGAAACCTCTTTGGTACACCATGATCCTCACCAGCAAAATCATCA | |
| GCCTCATTGCAGGCATTGCTGTGCTGAGGAGCTTGTACATGGTCATTCCACTGGTGTTTCT | |
| CCTCTTAAGGTTGCCCTTCTGTGGACATCGTATCATCCCTCATACTTACTGTGAGGACATGG | |
| GCATTGCCCGTCTGGCCTGTGCCAGCATCAAAGTCAACATTATGTTTGGTCTTGGCAGTAT | |
| TTCTCTCTTGTTATTGGATGTGCTCCTTATTATTCTCTCCCATATCAGGATCCTCTATGCTGT | |
| CTTCTGCCTGCCCTCCTGGGAAGCTCGACTCAAAGCTCTCAACACCTGTGGCTCTCACATT | |
| GGTGTTATCTTAGCCTTCTACACCAGCATTVHTCTGTTTCTTTACACACTGCTTTGGCCAT | |
| GATATTCCCCAATATATCCACATTTTCTTGGCTAATCTATATGTGGTTGTTCCTCCGACCCT | |
| GAATCCTGTAATCTATGGGGTCAGAACCAAACATATTAGGGAGACAGTGCTGAGGATTTC | |
| TTCAAGACAGATCACTAA | |
| AOLFR328 sequences: | |
| MALGNHSTTEFLLLGLSADPNIRALLFVLFLGITLLTIMENLMLLLVIRADSCLHKPMTTFLSH | (SEQ ID NO:511) |
| LSFVDLCFSSVIVPKMLENLLSQRKTISVEGCLAQVEFVPVTAGTEACLLSGMATDRIIAAIRRP | |
| LLTGQIMGKQLTMHLVWGSWGLGFLDALINVLLAVNMVFCEATIHHTSTEMPSLLPLSCSDI | |
| SRSLIVLLCSTLLHGLGNFLLVFLSTTRIISTILSISSTSGRSKATSTCSAAILTAVTLTTGSGLLRHL | |
| MPNSGSPIELIFSVQTTVVTPMLNSLITSLKNKEVKVALTRTLEKTLQTTRR | |
| ATGGCCITGGGGAATCACAGCACCATCACCGAGTTCCTCCTCCTTGGGCTGTCTGCCGACC | (SEQ ID NO:512) |
| CCAACATCCGGGCTCTGCTCTTTGTGCTGTTCCTGGGGATTTACCTCCTGACCATAATGGA | |
| AAACCTGATGCTGCTGCTCGTGATCAGGGCTGATTCTTGTCTCCATAAGCCCATGTATTTCT | |
| TCCTGAGTCACCTCTCTTTTGTTGATCTCTGCTTCTCTTCAGTCATTGTGCCCAAGATGCTG | |
| GAGAACCTCCTGTCACAGAGGAAAACCATTTCAGTAGAGGGCTGCCTGGCTCAGGTCTTCT | |
| TTGTGTTTGTCACTGCAGGGACTGAAGCCTGCCTTCTCTCAGGGATGGCCTATGACCGCCA | |
| TGGTGCCATCCGCCGCCCACTACTTTATGGACAGATCATGGGTAAACAGCTGTATATGCAC | |
| CTTGTGTGGGGCTCATGGGQACTGGGCTTTCTGGACGCACTCATCAATGTCCTCCTAGCTG | |
| TAAACATGGTCTTTTGTGAAGCCAAAATCATTCACCACTACAGCTATGAGATGCCATCCCT | |
| CCTCCCTCTGTCCTGCTCTGATATCTCCAGAAGCCTCATCGTTTGCTCTGCTCCACTCTCC | |
| TACATGGGCTGGGAAACTTCCTTTTGGTCTTCTTATCCTACACCGGTATAATCTCTACCATC | |
| CTAAGCATCAGCTCTACCTCGGGCAGAAGCAAGGCCTTCTCCACCTGCTCTGCCCACCTCA | |
| CTGCAGTGACACTTTACTATGGCTCAGGTTTGCTCCGCCATCTCATGCCAAACTCAGGTTC | |
| CCCCATAGAGTTGATCTTCTCTGTGCAGTATACTGTAGTCACTGCCATGCTGAATTCCCTCA | |
| TCTATAGCCTGAAAAATAAGGAAGTGAAGGTAGCTCTGAAAAGAACTTTGGAAAAATATT | |
| TGCAATATACCAGACGTTGA | |

What is claimed:

1. An isolated nucleic acid sequence selected from the group consisting of:
(i) an isolated nucleotide sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510; and SEQ ID NO: 512, or a fragment thereof which comprises at least 75 nucleotides;
(ii) an isolated cDNA or an insoluble RNA transcribed therefrom that encodes a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11,SEQ ID NO: 13,SEQ ID NO: 15,SEQ ID NO: 17,SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111,SEQ ID NO: 113,SEQ ID NO: 115,SEQ ID NO: 117,SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: , 263, SEQ ID NO: , 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409,SEQ ID NO: 411, SEQ ID NO: 413, SEQ ID NO: 415, SEQ ID NO: 417,SEQ ID NO: 419,SEQ ID NO: 421, SEQ ID NO: 423,SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511, or a fragment thereof which encodes at least 25 contiguous amino acids of said polypeptide;
(iii) a nucleic acid sequence that comprises at least 30% sequence identity with an isolated nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512, or to a fragment thereof which comprises at least 100 contiguous nucleotides thereof:
(iv) a nucleic acid sequence that encodes a polypeptide having at least 40% sequence identity at the amino acid level with a polypeptide having an amino acid sequence selected from the group consisting of: SEQ ID NO: 1, SEQ ID NO: 3, SEQ ID NO: 5,SEQ ID NO: 7,SEQ ID NO: 9, SEQ ID NO: 11, SEQ ID NO: 13,SEQ ID NO: 15,SEQ ID NO: 17,SEQ ID NO: 19,SEQ ID NO: 21,SEQ ID NO: 23,SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: , 263, SEQ ID NO: , 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ IDD NO: 291, SEQ ID NO: 293, SEQ ID NO: 295, SEQ ID NO: 297, SEQ ID NO: 299, SEQ ID D NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ D NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413,SEQ ID NO: 415,SEQ ID NO: 417,SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a nucleic acid sequence encoding at least 50 contiguous amino acid residues thereof;
(v) an isolated nucleic acid sequence which encodes an olfactory receptor or a fragment thereof that specifically hybridizes and exhibits at least 30% sequence identity under stringent conditions to a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ BD NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504,
SEQ ID NO: 506, SEQ ID NO: 508, SEQ ID NO: 510 and SEQ ID NO: 512;
(vi) an isolated nucleic acid sequence that specifically hybridizes to (i) or a portion thereof under stringent hybridization conditions that is at least 20-30 nucleotides in length; and
(vii) a naturally occurring allelic or synthetic variant of a nucleic acid sequence according to (i) or (ii), containing at least one substitution, deletion or addition mutation in the coding region.

2. The isolated nucleic acid sequence of claim 1 which is selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204.H SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a fragment thereof which comprises at least 75 contiguous nucleotides thereof.

3. The isolated nucleic acid sequence of claim 1 which encodes a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: , 263, SEQ ID NO: , 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381 SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof encoding at least 25 contiguous amino acid residues of said polypeptide.

4. An isolated nucleic acid sequence having at least 30-60% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102,SEQ ID NO: 104,SEQ ID NO: 106, SEQ ID NO: 108,SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512; or a fragment thereof comprising at least 100 contiguous nucleotides of any of said sequences.

5. An isolated nucleic acid sequence having at least 60-80% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a fragment thereof comprising at least 100 contiguous nucleotides of any of said sequences.

6. An isolated nucleic acid sequence having at least 80-90% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110 , SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a fragment thereof comprising at least 100 contiguous nucleotides of any of said sequences.

7. An isolated nucleic acid sequence having at least 85% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ NO: 108, SEQ ID NO: 100, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512, or a fragment thereof comprising at least 100 contiguous nucleotides of any of said sequences.

8. An isolated nucleic acid sequence having at least 90% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a fragment thereof comprising at least 100 contiguous nucleotides of any of said sequences.

9. An isolated nucleic acid sequence according to claim 1 which encodes a polypeptide having at least 40-60% sequence identity with a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: , 263, SEQ ID NO: , 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof that comprises at least 40 contiguous amino acids thereof.

10. An isolated nucleic acid sequence according to claim 1 which encodes a polypeptide having at least 60-70% sequence identity with a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103,SEQ ID NO: 105,SEQ ID NO: 107,SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: , 263, SEQ ID NO: , 265, SEQ ID NO: 267, SEQ ID NO: 269, SEQ ID NO: 271, SEQ ID NO: 273, SEQ ID NO: 275, SEQ ID NO: 277, SEQ HD NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof that comprises at least 40 contiguous amino acids thereof.

11. An isolated nucleic acid sequence according to claim 1 which encodes a polypeptide having at least 70-80% sequence identity with a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof that comprises at least 40 contiguous amino acids thereof.

12. An isolated nucleic acid sequence according to claim 1 which encodes a polypeptide having at least 80-90% sequence identity with a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111 , SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof that comprises at least 40 contiguous amino acids thereof.

13. An isolated nucleic acid sequence according to claim 1 which encodes a polypeptide having about 90-99% sequence identity with a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ D NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111 , SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof that comprises at least 40 contiguous amino acids thereof.

14. An isolated nucleic acid sequence which exhibits at least 50% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ IfD NO: 444, SEQ IID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ IID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ IID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ IID NO: 504, SEQ IID NO: 506, SEQ ID NO: 508, SEQ ID NO: 510 and SEQ ID NO: 512 or a nucleic acid sequence which exhibits at least 50% sequence identity to a fragment comprising at least 100 contiguous nucleotides of said nucleic acid sequence.

15. An isolated nucleic acid sequence which exhibits at least 60% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100 SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence which exhibits at least 60% sequence identity to a fragment comprising at least 100 contiguous nucleotides of said nucleic acid sequence.

16. An isolated nucleic acid sequence that exhibits at least 70% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID 10 NO: 92, SEQ ID NO: 94, SEQ ID NO: 96, SEQ ID NO: 98, SEQ ID NO: 100, SEQ ID NO: 102,SEQ ID NO: 104,SEQ ID NO: 106,SEQ ID NO: 108,SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID 25 NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence having at least 70% sequence identity with a fragment thereof comprising at least 100 contiguous nucleotides thereof.

17. An isolated nucleic acid sequence that exhibits at least 80% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence having at least 80% sequence identity with a fragment thereof comprising at least 100 contiguous nucleotides thereof.

18. An isolated nucleic acid sequence that exhibits at least 85% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence having at least 85% sequence identity with a fragment thereof comprising at least 100 contiguous nucleotides thereof.

19. An isolated nucleic acid sequence that exhibits at least 90% sequence identity with a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence having at least 90% sequence identity with a fragment thereof comprising at least 100contiguous nucleotides thereof.

20. An isolated nucleic acid sequence that exhibits at least 95% sequence identity with a nucleic acid sequence selected from the group consisting of: SEQ ID NO: 2, SEQ I=NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID
NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID
NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID 30 NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328=SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a nucleic acid sequence having at least 95% sequence identity with a fragment thereof comprising at least 100 contiguous nucleotides thereof.

21. An isolated nucleic acid sequence that exhibits about 96-99% sequence identity with a nucleic acid sequence encoding an olfactory receptor selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512 or a fragment having at least 96-99% sequence identity with a fragment thereof comprising at least 100 contiguous nucleotides thereof.

22. A nucleic acid sequence which encodes for a functional olfactory receptor polypeptide, wherein said nucleic acid sequence comprises a portion which is at least 100 nucleotides in length and exhibits at least 40% sequence identity with at least 100 contiguous nucleotides of a portion of an olfactory receptor encoding a nucleic acid sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104,SEQ ID NO: 106,SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512.

23. The nucleic acid sequence of claim 22 which is a chimeric nucleic acid sequence, wherein said nucleic acid sequence is produced by combining portions of at least two different G protein-coupled receptors.

24. The chimeric nucleic acid sequence of claim 23 wherein said two different G protein-coupled receptors are olfactory receptors.

25. The chimeric nucleic acid sequence of claim 23 wherein said chimeric sequence contains at least 200 contiguous nucleotides that are at least 40% identical to a portion of one of said olfactory receptor encoding nucleic acid sequences.

26. An isolated nucleic acid sequence according to claim 1, wherein said isolated nucleic acid sequence is directly or indirectly attached to a nucleic acid sequence that encodes a detectable polypeptide.

27. The nucleic acid sequence of claim 26, wherein said detectable polypeptide is green fluorescent protein, or a fragment or variant thereof.

28. An isolated nucleic acid sequence which encodes a polypeptide that exhibits at least 40% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ iD NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249,.SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

29. An isolated nucleic acid sequence which encodes a polypeptide that exhibits at least 50% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 10, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

30. An isolated nucleic acid sequence which encodes a polypeptide that exhibits at least 60% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

31. An isolated nucleic acid sequence which encodes a polypeptide that exhibits at least 70% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 10, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

32. An isolated nucleic acid sequence which encodes a polypeptide that exhibits at least 80% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO-445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

33. An isolated nucleic acid sequence which encodes a polypeptide that exhibits at least 85% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

34. An isolated nucleic acid sequence which encodes a polypeptide that exhibits at least 90% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ E) NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

35. An isolated nucleic acid sequence which encodes a polypeptide that exhibits about 90-99% sequence identity with a polypeptide selected from the group consisting of: SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ I NO: 507, SEQ ID NO: 509 and SEQ ID NO: 511 or a fragment thereof comprising at least 40 contiguous amino acids thereof that optionally is directly or indirectly attached to a sequence that facilitates the expression and/or translocation of said polypeptide on the surface of a cell.

36. The isolated nucleic acid sequence according to claim 26, wherein said isolated nucleic acid sequence is operably linked to a constitutive promoter.

37. The isolated nucleic acid sequence according to claim 1, wherein said isolated nucleic acid sequence is operably linked to a regulatable promoter.

38. The isolated nucleic acid sequence of claim 1, wherein said isolated nucleic acid sequence is directly or indirectly attached to a nucleic acid sequence encoding a mammalian rhodopsin polypeptide or a fragment thereof.

39. An isolated nucleic acid molecule comprising a nucleotide sequence that encodes a fragment of at least 60 contiguous amino acids of a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, and SEQ ID NO: 511.

40. The isolated nucleic acid molecule of claim 39, wherein the nucleotide sequence encodes at least 100 amino acids.

41. The isolated nucleic acid molecule of claim 39, wherein the nucleotide sequence encodes at least 150 amino acids.

42. The isolated nucleic acid molecule of claim 39, wherein the nucleotide sequence encodes at least 200 amino acids.

43. The isolated nucleic acid molecule of claim 39, wherein the nucleotide sequence encodes at least 250 amino acids.

44. The isolated nucleic acid molecule of claim 39, wherein the polypeptide is an olfactory G protein-coupled receptor.

45. The isolated nucleic acid molecule of claim 39, wherein the expression product binds an odorant.

46. The isolated nucleic acid molecule of claim 1 comprising a nucleotide sequence selected from the group consisting of: SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ iD NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, SEQ ID NO: 178, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204. SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ. ID NO. 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510 and SEQ ID NO: 512.

47. An expression vector that comprises a nucleic acid sequence according to claim 1.

48. The expression vector of claim 47, wherein said vector is a mammalian, yeast, bacterial or insect expression vector.

49. A cell which is transfected or transformed with at least one nucleic acid sequence according to claim 1.

50. A mammalian cell according to claim 49.

51. A human cell according to claim 50.

52. A yeast or insect cell according to claim 49.

53. The mammalian cell according to claim 49 which is selected from the group consisting of: an olfactory cell, Chinese hamster ovary cell, baby hamster kidney cell, and a myeloma cell.

54. A solid phase comprising at least one isolated nucleic acid sequence according to claim 1.

55. A solid phase comprising at least one isolated nucleic acid sequence according to claim 1, wherein the solid phase is attached to an array comprising at least one additional nucleic acid sequence.

56. The solid phase according to claim 55 which comprises an array of at least 4 different nucleic acid sequences that encode olfactory receptors or fragments or variants thereof.

57. The solid phase according to claim 55 which comprises at least 10 different nucleic acid sequences that encode olfactory receptors or fragments or variants thereof.

58. The solid phase according to claim 55 which comprises at least 50 different nucleic acid sequences that encode olfactory receptors or fragments or variants thereof.

59. The solid phase according to claim 55 which comprises at least 100 different sequences that encode olfactory receptors or fragments or variants thereof.

60. An isolated polypeptide that is selected from the group consisting of:
(i) a polypeptide comprising an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119,SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID 30 NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511;
(ii) a polypeptide comprising an amino acid sequence that exhibits at least 40% sequence identity with an amino acid sequence selected from the group consisting of: SEQ ID NO: 1, SEQ ID NO: 3,SEQ ID NO: 5, SEQ ID NO: 7,SEQ ID NO: 9, SEQ ID NO: 11l, SEQ ID NO: 13,SEQ ID NO: 15,SEQ ID NO: 17,SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81,SEQ ID NO: 83,SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: O1, SEQ ID NO: 103,SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155,SEQ ID NO: 157,SEQ ID NO: 159,SEQ ID NO: 161,SEQ ID NO: 163, SEQ ID NO: 165, SEQ ID NO: 167, SEQ ID NO: 169, SEQ ID NO: 171, SEQ ID NO: 173,SEQ ID NO: 175,SEQ ID NO: 177,SEQ ID NO: 179,SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213,SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ IDNO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511:
(iii) a polypeptide comprising an amino acid sequence that exhibits at least 60% sequence identity with a fragment of a polypeptide according to (i) which fragment is at least 40 amino acids in length;
(iv) a chimeric polypeptide that comprises a portion of a polypeptide according to (i) or (ii) that is at least 40 amino acids in length and a portion of at least one other G protein-coupled receptor; and
(v) a variant of a polypeptide according to (i) which differs by said polypeptide by at least one substitution, addition or deletion modification.

61. An isolated polypeptide according to claim 60 wherein such polypeptide exhibits at least 70% sequence identity with a polypeptide having a sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 amino acids.

62. An isolated polypeptide according to claim 60 wherein said polypeptide exhibits at least 80% sequence identity with a polypeptide having a sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 11, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 amino acids.

63. An isolated polypeptide according to claim 60 wherein said polypeptide exhibits at least 90% sequence identity with a polypeptide having a sequence selected from the group consisting of: SEQ ID NO: 1, SEQ ID NO: 3, SEQ ID NO: 5,SEQ ID NO: 7,SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105,SEQ ID NO: 107,SEQ ID NO: 109,SEQ ID NO: 111,SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 amino acids.

64. An isolated polypeptide according to claim 60 wherein said polypeptide exhibits about 80-90% sequence identity with a polypeptide having a sequence selected from the group consisting of: SEQ ID NO: 1, SEQ ID NO: 3, SEQ ID NO: 5, SEQ ID NO: 7, SEQ ID NO: 9, SEQ ID=NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105,SEQ ID NO: 107,SEQ ID NO: 109,SEQ ID NO: 111,SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 arnino acids.

65. An isolated polypeptide according to claim 60 wherein said polypeptide exhibits at least 90-95% sequence identity with a polypeptide having a sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47,SEQ ID NO: 49, SEQ ID NO: 51,SEQ ID NO: 53,SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105,SEQ ID NO: 107,SEQ ID NO: 109,SEQ ID NO: 111,SEQ ID NO: 113, SEQ ID NO: 115,SEQ ID NO: 117,SEQ ID NO: 119, SEQ ID NO: 121,SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 amino acids.

66. An isolated polypeptide according to claim 60 wherein said polypeptide exhibits about 95-99% sequence identity with a polypeptide having a sequence selected from the group consisting of: SEQ ID NO: 1, SEQ ID NO: 3, SEQ ID NO: 5, SEQ ID NO: 7, SEQ ID NO: 9, SEQ ID NO: 1, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105,SEQ ID NO: 107,SEQ ID NO: 109,SEQ ID NO: 11,SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511 or a fragment thereof which is at least 50 amino acids.

67. A variant according to claim 60(v) which comprises at least 5 conservative amino acid substitutions.

68. A variant according to claim 60(v) which comprises at most 5 conservative amino acid substitutions.

69. A variant according to claim 60(v) which comprises 5 to 7 conservative substitution modifications.

70. A variant according to claim 60(v) which comprises 3 to 4 conservative substitution modifications.

71. A variant according to claim 60(v) which comprises 1 or 2 conservative substitution modifications.

72. A solid phase comprising at least one directly or indirectly immobilized isolated polypeptide according to claim 60, or a cell which expresses said polypeptide on the surface thereof.

73. The solid phase of claim 72 comprising at least 4 different immobilized polypeptides according to claim 60, or a cell which expresses said polypeptide on the surface thereof.

74. The solid phase of claim 72 comprising at least 16 different immobilized polypeptides according to claim 60, or a cell which expresses said polypeptide on the surface thereof.

75. The solid phase of claim 72 comprising at least 25 different immobilized polypeptides according to claim 60 or a cell which expresses said polypeptide on the surface thereof.

76. A method of detecting expression of an olfactory receptor gene comprising (a) hybridizing at least one sample with a nucleic acid according to claim 1 and (b) detecting expression of the olfactory receptor gene by a positive hybridization signal.

77. A method of screening a library comprising (a) hybridizing the library with a nucleic acid according to claim 1 and (b) detecting one or more olfactory receptor clones in the library by a positive hybridization signal.

78. A recombinant polynucleotide comprising a nucleic acid according to claim 1 attached directly or indirectly to a heterologous nucleic acid.

79. An expression vector comprising the nucleic acid of claim 1 and an operably linked heterologous nucleic acid that drives expression thereof.

80. A transfected or transformed cell comprising the recombinant polynucleotide of claim 78 introduced into a host cell, or a progeny thereof.

81. A transgenic non-human organism comprising the recombinant polynucleotide of claim 78 introduced into a cell of a host non-human organism, or a progeny thereof.

82. A method of making a recombinant polynucleotide comprising ligating the nucleic acid of claim 1 to a heterologous nucleic acid.

83. The method of claim 82 wherein the heterologous nucleic acid comprises a translational and/or transcriptional regulatory region.

84. A method of making a transfected cell comprising introducing the recombinant polynucleotide of claim 79 into a host cell, and propagating the host cell in which the recombinant polynucleotide has been introduced.

85. A method of detecting specific binding of a putative ligand to an olfactory receptor comprising (a) contacting the putative ligand with a cell in which the expression vector of claim 79 has been introduced, wherein the olfactory receptor is expressed by the cell thereby, and (b) directly or indirectly detecting specific binding between the putative ligand and the olfactory receptor.

86. A method of making transgenic non-human organism comprising introducing the recombinant polynucleotide of claim 78 into a cell of a host non-human organism, or propagating the host non-human organism in which the recombinant polynucleotide has been introduced.

87. An isolated protein molecule comprising a fragment of at least 60 contiguous amino acids of a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ IID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

88. The isolated protein molecule of claim 87, wherein the fragment contains at least 100 amino acids.

89. The isolated protein molecule of claim 87, wherein the fragment contains at least 150 amino acids.

90. The isolated protein molecule of claim 87, wherein the fragment contains at least 200 amino acids.

91. The isolated protein molecule of claim 87, wherein the fragment contains at least 250 amino acids.

92. The isolated protein molecule of claim 87, which is a functional olfactory receptor polypeptide.

93. The isolated protein molecule of claim 87, wherein the fragment specifically binds an odorant molecule.

94. A recombinant polypeptide comprising the protein molecule of claim 87 and a heterologous peptide domain.

95. The recombinant polypeptide of claim 94, wherein the heterologous peptide domain comprises a G protein-coupled receptor transmembrane domain.

96. The recombinant polypeptide of claim 94 comprising a seven-transmembrane receptor with an olfactory receptor ligand-binding domain, wherein the olfactory receptor ligand-binding domain is a chimera of at least two different olfactory receptors.

97. A method of detecting specific binding of a ligand to an olfactory receptor comprising (a) contacting the ligand with the protein of claim 86, and (b) directly or indirectly detecting specific binding between the ligand and the olfactory receptor.

98. An antibody or antibody fragment that specifically binds a polypeptide having an amino acid sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111,SEQ ID NO: 113,SEQ ID NO: 115,SEQ ID NO: 117,SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ HD NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ED NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

99. A method of detecting specific binding of the antibody of claim 98 to an olfactory receptor comprising (a) contacting the antibody with a sample comprising the olfactory receptor and (b) detecting specific binding therebetween.

100. The method of claim 99, wherein specific binding of the antibody to a cell in the sample identifies the cell as an olfactory cell.

101. A method of screening a library of chemical compounds for compounds that are involved in olfactory sensation comprising contacting compounds in said library with at least one polypeptide according to claim 87 and identifying compounds that specifically bind to at least one of said polypeptides.

102. The method of claim 101 wherein said library is a combinatorial chemical library.

103. The method of claim 101 wherein said library is a peptide library.

104. The method of claim 101 wherein said library is a peptide, encoded peptide, benzodiazepine, diversomer, vinylogous polypeptide, nonpeptidal peptidominetic, or small molecule organic compound library.

105. The method of claim 101 wherein said library is a random combination of compounds.

106. The method of claim 101 wherein said compounds are screened by high turning point screening.

107. The method of claim 101 wherein said screening is effected using animal cells or tissues that express at least one of said polypeptides.

108. A cell-based assay for identifying molecules that interact with an olfactory receptor comprising:
obtaining a cell that expresses at least one polypeptide according to claim 60 or a chimeric protein comprising a portion of said protein and that of another G protein-coupled receptor, and which optionally expresses at least one functional G protein;
contacting said cell with a molecule to be screened for its ability to modulate an olfactory receptor; and
detecting whether modulation occurs.

109. The method of claim 108 wherein modulation is detected based on changes in intracellular calcium.

110. The method of claim 108 wherein modulation is detected by measuring the transfer of 32P from gamma-labeled GTP to the olfactory receptor polypeptide.

111. The method of claim 108 wherein modulation is determined based on a comparison to a control compound known to modulate the particular olfactory receptor protein.

112. The method of claim 108 wherein the G protein is Gα15 or Gα16 or another promiscuous G protein.

113. The method of claim 108 wherein modulation is determined by detecting whether a change in the level of intracellular cyclic nucleotides occurs.

114. The method of claim 108 wherein modulation is determined based on the level of transcription of said olfactory polypeptide after contacting the cell with the screened compound.

115. The method of claim 108 when said screened compounds are synthesized by computer assisted drug devices based on the predicted or actual three-dimensional structure of the amino acid sequence of the olfactory protein or a fragment thereof.

116. The method of claim 108 wherein compounds that modulate olfactory receptor are identified based on whether they specifically bind to a olfactory receptor polypeptide.

117. The method of claim 108 wherein modulation refers to the inhibition of olfactory receptor function.

118. The method of claim 108 wherein modulation refers to the enhancement of olfactory receptor function.

119. A method for representing the olfactory perception of one or more odors in one or more mammals, comprising:
providing values X₁ to Xₙ representative of the quantitative stimulation of each of n odor receptors of said mammals; and
generating from said values a quantitative representation of odor perception, wherein at least one of said odor receptors is an odor receptor polypeptide having a sequence that is at least about 40% identical to a sequence selected from the group consisting of: SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75=SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109,SEQ ID NO: 111,SEQ ID NO: 113,SEQ ID NO: 115,SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 91, SEQ ID NO: 3, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ D NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ IID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.

120. The method of claim 119, wherein said representation constitutes a point or a volume in n-dimensional space.

121. The method of claim 119, wherein said representation constitutes a graph or a spectrum.

122. The method of claim 119, wherein said representation constitutes a matrix of quantitative representations.

123. The method of claim 119, wherein said providing step comprises contacting a plurality of recombinantly produced olfactory receptors with a test composition, and quantitatively measuring the interaction of said composition with said receptors.

124. A method for predicting the odor perception in a mammal generated by one or more molecules or combinations of molecules comprising:
providing values X₁ to Xₙ representative of the quantitative stimulation of each of n odor receptors of said mammal, for one or more molecules or combinations of molecules yielding known odor perception in a mammal,
generating from said values a quantitative representation of odor perception in a mammal for the one or more molecules or combinations of molecules yielding known odor perception in a mammal;
providing values X₁ to Xₙ representative of the quantitative stimulation of each of n odor receptors of said mammal, for one or more molecules or combinations of molecules yielding unknown odor perception in a mammal;
generating from said values a quantitative representation of odor perception in a mammal for the one or more molecules or combinations of molecules yielding unknown odor perception in a mammal; and
predicting the olfactory perception in a mammal generated by one or more molecules or combinations of molecules yielding unknown odor perception in a mammal by comparing the quantitative representation of odor perception in a mammal generated by one or more molecules or combinations of molecules yielding unknown odor perception in a mammal to the quantitative representation of odor perception in a mammal for the one or more molecules or combinations of molecules yielding known odor perception in a mammal, wherein at least one of said odor receptors is a odor receptor polypeptide having a sequence that is at least about 40% identical to a sequence selected from the group consisting of: SEQ ID NO: 1, SEQ ID NO: 3, SEQ ID NO: 5,SEQ ID NO: 7, SEQ ID NO: 9,SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO:, 263, SEQ ID NO:, 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509 and SEQ ID NO: 511.
